(19) United States
(12) Patent Application Publication
(10) Pub. No.: US 2025/0223371 A1
(43) Pub. Date: Jul. 10, 2025

(54) EXPANSION OF TUMOR INFILTRATING LYMPHOCYTES (TILS) WITH TUMOR NECROSIS FACTOR RECEPTOR SUPERFAMILY (TNFRSF) AGONISTS AND THERAPEUTIC COMBINATIONS OF TILS AND TNFRSF AGONISTS

(71) Applicant: Iovance Biotherapeutics, Inc., San Carlos, CA (US)

(72) Inventors: Michael T. Lotze, Pittsburgh, PA (US); Krit Ritthipichai, Tampa, FL (US)

(21) Appl. No.: 18/429,006

(22) Filed: Jan. 31, 2024

Related U.S. Application Data
(63) Continuation of application No. 17/196,018, filed on Mar. 9, 2021, now abandoned.
Application No. 17/196,018 is a continuation of application No. 16/475,924, filed on Jul. 3, 2019, now abandoned.
(60) Provisional application No. 62/443,556, filed on Jan. 6, 2017.
(60) Provisional application No. 62/460,477, filed on Feb. 17, 2017.
(60) Provisional application No. 62/532,807, filed on Jul. 14, 2017.
(60) Provisional application No. 62/567,151, filed on Oct. 2, 2017.

(51) Int. Cl. C07K 16/28; A61K 40/11; A61K 40/42; A61K 45/06; A61P 35/00; C12N 5/078; C12N 5/0783

(52) U.S. Cl. CPC C07K 16/2875; A61K 40/11; A61K 40/42; A61P 35/00; C07K 16/2878; C12N 5/0634; C12N 5/0636; A61K 45/06; A61K 2239/54; A61K 2239/59

(57) ABSTRACT

Methods of expanding tumor infiltrating lymphocytes (TILs) using a tumor necrosis factor receptor superfamily (TNFRSF) agonist, such as a 4-1BB agonist, a CD27 agonist, a glucocorticoid-induced TNF receptor-related agonist, an OX40 agonist, a HVEM agonist, or a CD95 agonist, and uses of such expanded TILs in the treatment of diseases such as cancer are disclosed herein. In addition, in some embodiments, therapeutic combinations of TILs and TNFRSF agonists useful in the treatment of diseases such as cancer, including compositions, uses, and dosing regimens thereof, are disclosed herein.

### CROSS-REFERENCE TO RELATED APPLICATIONS

This application is a continuation of U.S. application Ser. No. 17/196,018, filed Mar. 9, 2021, which is a continuation of U.S. application Ser. No. 16/475,924, filed Jul. 3, 2019, which is a U.S. National Stage application of International Application No. PCT/US2018/012605, filed Jan. 5, 2018, which claims priority to U.S. Provisional Application No. 62/443,556, filed Jan. 6, 2017, U.S. Provisional Application No. 62/460,477, filed Feb. 17, 2017, U.S. Provisional Application No. 62/532,807, filed Jul. 14, 2017, and U.S. Provisional Application No. 62/567,151, filed Oct. 2, 2017, the entirety of which are incorporated herein by reference.

### SEQUENCE LISTING

The instant application contains a Sequence Listing which has been submitted electronically in XML format and is hereby incorporated by reference in its entirety. Said XML copy, created on Nov. 5, 2024, is named 116983-5008-WO_Sequence_Listing.xml and is 707,707 bytes in size.

### FIELD OF THE INVENTION

Methods of expanding tumor infiltrating lymphocytes (TILs) using a tumor necrosis factor receptor superfamily (TNFRSF) agonist, such as a 4-1BB agonist, a CD27 agonist, a glucocorticoid-induced TNF receptor-related agonist, an OX40 agonist, a HVEM agonist, or a CD95 agonist, and uses of expanded TILs in the treatment of diseases such as cancer are disclosed herein. In addition, therapeutic combinations of TILs and TNFRSF agonists, including compositions and uses thereof in the treatment of diseases such as cancer are disclosed herein.

### BACKGROUND OF THE INVENTION

Treatment of bulky, refractory cancers using adoptive autologous transfer of tumor infiltrating lymphocytes (TILs) represents a powerful approach to therapy for patients with poor prognoses. Gattinoni, et al., Nat. Rev. Immunol. 2006, 6, 383-393. TILs are dominated by T cells, and IL-2-based TIL expansion followed by a “rapid expansion process” (REP) has become a preferred method for TIL expansion because of its speed and efficiency. Dudley, et al., Science 2002, 298, 850-54; Dudley, et al., J. Clin. Oncol. 2005, 23, 2346-57; Dudley, et al., J. Clin. Oncol. 2008, 26, 5233-39; Riddell, et al., Science 1992, 257, 238-41; Dudley, et al., J. Immunother. 2003, 26, 332-42. A number of approaches to improve clinical responses to TIL therapy in melanoma and to expand TIL therapy to other tumor types have been explored with limited success, and the field remains challenging. Goff, et al., J. Clin. Oncol. 2016, 34, 2389-97; Dudley, et al., J. Clin. Oncol. 2008, 26, 5233-39; Rosenberg, et al., Clin. Cancer Res. 2011, 17, 4550-57. Much focus has been placed on selection of TILs during expansion to either select particular subsets (such as CD8⁺ T cells) or to target driver mutations such as a mutated ERBB2IP epitope or driver mutations in the KRAS oncogene. Tran, et al., N. Engl. J. Med. 2016, 375, 2255-62; Tran, et al., Science 2014, 344, 641-45. However, such selection approaches, even if they can be developed to show efficacy in larger clinical trials, add significantly to the duration, complexity, and cost of performing TIL therapy and limit the potential for widespread use of TIL therapy in different types of cancers.

4-1BB (also known as CD137 and TNFRSF9), which was first identified as an inducible costimulatory receptor expressed on activated T cells, is a membrane spanning glycoprotein member of the TNFRSF. Watts, Annu. Rev. Immunol. 2005, 23, 23-68. 4-1BB is a type 2 transmembrane glycoprotein that is expressed on activated T lymphocytes, and to a larger extent on CD8⁺ than CD4⁺ T cells. 4-1BB is also expressed on dendritic cells, follicular dendritic cells, natural killer (NK) cells, granulocytes, cells of blood vessel walls at sites of inflammation, tumor vasculature, and atherosclerotic endothelium. The ligand that stimulates 4-1BB (4-1BBL) is expressed on activated antigen-presenting cells (APCs), myeloid progenitor cells and hematopoietic stem cells. 4-1BB is an activation-induced T-cell costimulatory molecule. Signaling through 4-1BB upregulates survival genes, enhances cell division, induces cytokine production, and prevents activation-induced sell death in T cells. Current understanding of 4-1BB indicates that expression is generally activation dependent and encompasses a broad subset of immune cells including activated NK and NK T cells (NKT cells); regulatory T cells; dendritic cells (DC) including follicular DCs; stimulated mast cells, differentiating myeloid cells, monocytes, neutrophils, eosinophils, and activated B cells. 4-1BB strongly enhances the proliferation and effector function of CD8⁺ T cells. Crosslinking of 4-1BB enhances T cell proliferation, IL-2 secretion survival and cytolytic activity. Additionally, anti-4-1BB monoclonal antibodies possess strong antitumor properties, which in turn are the result of their powerful CD8+ T-cell activating, IFN-g producing, and cytolytic marker-inducing capabilities. Vinay and Kwon, Mol. Cancer Therapeutics 2012, 11, 1062-70; Lee, et al., PLoS One, 2013, 8, e69677, 1-11.

Interaction of 4-1BB on activated normal human B cells with its ligand at the time of B cell receptor engagement stimulates proliferation and enhances survival. The potential impact of 4-1BB engagement in B cell lymphoma has been investigated in at least two published studies. Evaluation of several types of human primary NHL samples indicated that 4-1BB was expressed predominantly on infiltrating T cells rather than the lymphoma cells. Houot, et al., Blood, 2009, 114, 3431-38. The addition of 4-1BB agonists to in vitro cultures of B lymphoma cells with, rituximab and NK cells resulted in increased lymphoma killing. Kohrt, et al., Blood, 2011, 117, 2423-32. In addition, B cell immunophenotyping was performed in two experiments using PF-05082566 in cynomolgus monkeys with doses from 0.001-100 mg/kg; in these experiments peripheral blood B cell numbers were either unchanged or decreased, as described in International Patent Application Publication No. WO 2015/119923.

4-1BB is undetectable on the surface of naïve T cells but expression increases upon activation. Upon 4-1BB activation, two pro-survival members of the TNFR-associated factor (TRAF) family, TRAF1 and TRAF2, are recruited to the 4-1BB cytoplasmic tail, resulting in downstream activation of NFkB and the Mitogen Activated Protein (MAP) kinase cascade including Erk, Jnk, and p38 MAP kinases. NFkB activation leads to upregulation of Bfl-1 and Bel-XL, pro-survival members of the Bcl-2 family. The pro-apoptotic protein Bim is downregulated in a TRAF1 and Erk dependent manner. Sabbagh, et al., J. Immunol. 2008, 180, 8093-8101. Reports have shown that 4-1BB agonist monoclonal antibodies (mAbs) increase costimulatory molecule expression and markedly enhance cytolytic T lymphocyte responses, resulting in anti-tumor efficacy in various models. 4-1BB agonist mAbs have demonstrated efficacy in prophylactic and therapeutic settings and both monotherapy and combination therapy tumor models and have established durable anti-tumor protective T cell memory responses. Lynch, et al., Immunol Rev., 2008, 222, 277-286. 4-1BB agonists also inhibit autoimmune reactions in a variety of autoimmunity models. Vinay, et al., J. Mol. Med. 2006, 84, 726-36.

The OX40 receptor (OX40) (also known as TNFRSF4, CD134, ACT-4, and ACT35) is a member of the TNF receptor family which is expressed on activated CD4⁺ T cells (see WO 95/12673). Triggering of this receptor via the OX40 ligand, named OX40L, gp34 or ACT-4-ligand, which is present on activated B-cells and dendritic cells, enhances the proliferation of CD4⁺ T cells during an immune response and influences the formation of CD4⁺ memory T-cells. Furthermore, the OX40-OX40L system mediates adhesion of activated T cells to endothelial cells, thus directing the activated CD4⁺ T cells to the site of inflammation.

It has been shown that OX40⁺ T cells are present within tumor lesions containing tumor infiltrating lymphocytes and in tumor cell positive draining lymph nodes. Weinberg, et al., J. Immunol., 2000, 164, 2160-2169. It was shown in several tumor models in mice that engagement of the OX40 receptor in vivo during tumor priming significantly delayed and prevented the appearance of tumors as compared to control treated mice. Weinberg, et al., J. Immunol., 2000, 164, 2160-2169. Hence, it has been contemplated to enhance the immune response of a mammal to an antigen by engaging the OX40-receptor by administering an OX40-receptor binding agent (International Patent Application Publication No. WO 1999/042585; Weinberg, et al., J. Immunol., 2000, 164, 2160-2169). Preclinical studies demonstrated that treatment of tumor bearing hosts with OX40 agonists, including both anti-OX40 monoclonal antibodies and OX40L-Fc fusion proteins, resulted in tumor regression in several preclinical models. Linch, et al., Front. Oncol. 2015, 34, 1-14.

CD27, also known as TNFRSF7, has overlapping activity with other TNFRSF members including CD40, 4-1BB, and OX40. CD27 plays a critical role in T cell survival, activation, and effector function, and also plays a role in the proliferative and cytotoxic activity of NK cells. CD27 is constitutively expressed on the majority of T cells, including naïve T cells. The ligand for CD27 is CD70, which is found on T cells, B cells, and dendritic cells. Oshima, et al., Int. Immunol. 1998, 10, 517-26. CD27 drives the expansion of CD4⁺ and CD8⁺ T cells, acting after CD28 to sustain T effector cell survival, and influences secondary responses more than primary responses. However, CD27 activation has also been associated with tumor growth through enhancement of the immunosuppressive effects of regulatory T cells. Claus, et al., Cancer Res. 2012, 72, 3664-76. Other data has indicated that the immunostimulatory effects of CD27 may outweigh this tumor promoting effect. Aulwurm, et al., Int. J. Cancer 2006, 118, 1728-35. In mouse models, an agonistic CD27 monoclonal antibody showed antitumor efficacy and induction of tumor immunity. He, et al., J. Immunol. 2013, 191, 4174-83.

Glucocorticoid-induced TNFR-related protein (GITR) is a costimulatory checkpoint molecule that is also known as tumor necrosis factor receptor superfamily member 18 (TNFRSF18), activation-inducible TNFR family receptor (AITR), and CD357. GITR is expressed on several cell types, including regulatory T cells (Tregs) and effector T cells, B cells, NK cells, and antigen-presenting cells. Nocentini and Riccardi, Eur. J. Immunol. 2005, 35, 1016-1022. GITR is activated by its conjugate GITR ligand (GITRL). GITR plays a role in stimulating an immune response, and antigen binding proteins to GITR have utility in treating a variety of GITR-related diseases or disorders in which it is desirable to increase an immune response. Ko, et al., J. Exp. Med. 2005, 202, 885-91; Shimizu, et al., Nature Immunology 2002, 3, 135-142; Cohen, et al., Cancer Res. 2006, 66, 4904-12; Azuma, Crit. Rev. Immunol. 2010, 30, 547-57. For example, T cell stimulation through GITR attenuates Treg-mediated suppression and enhances tumor-killing by CD4⁺ and CD8⁺ T cells. GITR is constitutively expressed at high levels in Tregs (such as CD4⁺CD25⁺ or CD8⁺CD25⁺ cells) and is additionally upregulated upon activation of these cells. Nocentini and Riccardi, Eur. J. Immunol. 2005, 35, 1016-1022. GITR is a co-activating signal to both CD4⁺ and CD8⁺ naïve T cells, and induces and enhances proliferation and effector function, particularly in situations where T cell receptor (TCR) stimulation is suboptimal. Schaer, et al., Curr. Opin. Immunol. 2012, 24, 217-224. The enhanced immune response caused by antigen binding GITR proteins, such as fusion proteins and anti-GITR antibodies (including agonistic antibodies), is of interest in a variety of immunotherapy applications, such as the treatment of cancers, autoimmune diseases, inflammatory diseases, or infections.

Herpesvirus entry mediator (HVEM), also known as TNFRSF14 and CD270, was first isolated as a receptor for herpes simplex virus-1 (HSV-1). Montgomery, et al., Cell 1996, 87, 427-36. HVEM binds to the TNF family ligands LIGHT and lymphotoxin alpha homotrimer (Ltα3). Mauri, et al., Immunity 1998, 8, 21-30. T cell activation can occur through the HVEM-LIGHT interaction, and the interaction provides a costimulatory signal to T cells that is independent of CD28 signaling and can be observed in the presence of suboptimal levels of CD3 antibody (OKT-3). Tamada, et al., J. Immunol. 2000, 165, 4397-404; Harrop, et al., J. Biol. Chem. 1998, 273, 27548-56; Tamada, et al., Nat. Med. 2000, 6, 283-89; Yu, et al., Nat. Immunol. 2004, 5, 141-49. HVEM comprises four cysteine-rich domains (CRDs). del Rio, et al., J. Leukoc. Biol. 2010, 87, 223-35. CRD2 and CRD3 are required for HVEM trimerization with the TNFRSF ligand LIGHT, which delivers a co-stimulatory signal to T cells through HVEM. In contrast, CRD1 and CRD2 bind to the co-inhibitory B and T lymphocyte attenuator (BTLA) receptor and CD160 in a monomeric manner, providing an inhibitory signal to T cells. Studies of the HVEM-LIGHT interaction suggest that it primarily has a CD28-independent costimulatory effect on CD8+ T cells, but also affects CD4+ T cells. Liu, et al., Int. Immunol. 2003, 15, 861-70; Scheu, et al., J. Exp. Med. 2002, 195, 1613-24.

CD95, also known as Fas, APO-1, and TNFRSF6, is a 45 kDa type-I transmembrane protein which, unlike 4-1BB, OX40, GITR, CD27, and HVEM, contains a death domain. Kischkel, et al., EMBO J. 1995, 14, 5579-88; Krammer, Nature 2000, 407, 789-95. The binding of the inducible CD95 ligand (CD95L) to CD95 on activated T cells leads to apoptotic cell death, and thus it is not normally associated with the same costimulatory function as 4-1BB, OX40, GITR, CD27, and HVEM. Strauss, et al., J. Exp. Med. 2009, 206, 1379-93. However, CD95 also behaves as a dual function receptor that provides for anti-apoptotic and costimulatory effects on T cells under some conditions. Paulsen, et al., Cell Death Differ. 2011, 18, 619-31. CD95 engagement modulates TCR-driven signal initiation in a dose-dependent manner, wherein high doses of CD95 agonists or cellular CD95L silence T cells, while lower doses of these agonists strongly enhance TCR-driven T cell activation and proliferation.

The present invention provides the unexpected finding that TNFRSF agonists, such as a 4-1BB agonist, a CD27 agonist, a GITR agonist, an OX40 agonist, a HVEM agonist, or a CD95 agonist, are useful in the expansion of TILs from tumors from which it is known to be difficult to obtain TILs and treat the tumor with TILs, and are further useful in the treatment of patients in combination with TIL therapy.

### SUMMARY OF THE INVENTION

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein the TNFRSF agonist is selected from the group consisting of a 4-1BB agonist, an OX40 agonist, a CD27 agonist, a GITR agonist, a HVEM agonist, a CD95 agonist, and combinations thereof.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein the TNFRSF agonist is a 4-1BB agonist, and the 4-1BB agonist is selected from the group consisting of urelumab, utomilumab, EU-101 and fragments, derivatives, variants, biosimilars, and combinations thereof.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein the TNFRSF agonist is a 4-1BB agonist, and the 4-1BB agonist is a 4-1BB agonist fusion protein.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein the TNFRSF agonist is a 4-1BB agonist fusion protein, and the 4-1BB agonist fusion protein comprises (i) a first soluble 4-1BB binding domain, (ii) a first peptide linker, (iii) a second soluble 4-1BB binding domain, (iv) a second peptide linker, and (v) a third soluble 4-1BB binding domain, further comprising an additional domain at the N-terminal and/or C-terminal end, and wherein the additional domain comprises a Fc fragment domain and hinge domain, and wherein the fusion protein is a dimeric structure according to structure I-A or structure I-B.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein the TNFRSF agonist is a OX40 agonist, and the OX40 agonist is selected from the group consisting of tavolixizumab, GSK3174998, MEDI6469, MEDI6383, MOXR0916, PF-04518600, Creative Biolabs MOM-18455, and fragments, derivatives, variants, biosimilars, and combinations thereof.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein the TNFRSF agonist is an OX40 agonist, and the OX40 agonist is an OX40 agonist fusion protein.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein the TNFRSF agonist is an OX40 agonist fusion protein, and the OX40 agonist fusion protein comprises (i) a first soluble OX40 binding domain, (ii) a first peptide linker, (iii) a second soluble OX40 binding domain, (iv) a second peptide linker, and (v) a third soluble OX40 binding domain, further comprising an additional domain at the N-terminal and/or C-terminal end, and wherein the additional domain comprises a Fc fragment domain and hinge domain, and wherein the fusion protein is a dimeric structure according to structure I-A or structure I-B.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein the TNFRSF agonist is a CD27 agonist, and the CD27 agonist is varlilumab, or a fragment, derivative, variant, or biosimilar thereof.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein the TNFRSF agonist is a CD27 agonist, and wherein the CD27 agonist is an CD27 agonist fusion protein.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein the TNFRSF agonist is a CD27 agonist, and the CD27 agonist fusion protein comprises (i) a first soluble CD27 binding domain, (ii) a first peptide linker, (iii) a second soluble CD27 binding domain, (iv) a second peptide linker, and (v) a third soluble CD27 binding domain, further comprising an additional domain at the N-terminal and/or C-terminal end, and wherein the additional domain comprises a Fc fragment domain and hinge domain, and wherein the fusion protein is a dimeric structure according to structure I-A or structure I-B.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein the TNFRSF agonist is a GITR agonist, and the GITR agonist is selected from the group consisting of TRX518, 6C8, 36E5, 3D6, 61G6, 6H6, 61F6, 1D8, 17F10, 35D8, 49A1, 9E5, 31H6, 2155, 698, 706, 827, 1649, 1718, 1D7, 33C9, 33F6, 34G4, 35B10, 41E11, 41G5, 42A11, 44C1, 45A8, 46E11, 48H12, 48H7, 49D9, 49E2, 48A9, 5H7, 7A10, 9H6, and fragments, derivatives, variants, biosimilars, and combinations thereof.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein the TNFRSF agonist is an GITR agonist, and the GITR agonist is a GITR agonist fusion protein.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein the TNFRSF agonist is a GITR agonist fusion protein, and the GITR agonist fusion protein comprises (i) a first soluble GITR binding domain, (ii) a first peptide linker, (iii) a second soluble GITR binding domain, (iv) a second peptide linker, and (v) a third soluble GITR binding domain, further comprising an additional domain at the N-terminal and/or C-terminal end, and wherein the additional domain comprises a Fc fragment domain and hinge domain, and wherein the fusion protein is a dimeric structure according to structure I-A or structure I-B.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein the TNFRSF agonist is a HVEM agonist.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein the TNFRSF agonist is an HVEM agonist, and the HVEM agonist is a HVEM agonist fusion protein.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein the TNFRSF agonist is a HVEM agonist fusion protein, and wherein the HVEM agonist fusion protein comprises (i) a first soluble HVEM binding domain, (ii) a first peptide linker, (iii) a second soluble HVEM binding domain, (iv) a second peptide linker, and (v) a third soluble HVEM binding domain, further comprising an additional domain at the N-terminal and/or C-terminal end, and wherein the additional domain comprises a Fc fragment domain and hinge domain, and wherein the fusion protein is a dimeric structure according to structure I-A or structure I-B.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, further comprising the step of treating the patient with the TNFRSF agonist starting on the day after administration of the third population of TILs to the patient, wherein the TNFRSF agonist is administered intravenously at a dose of between 0.1 mg/kg and 50 mg/kg every four weeks for up to eight cycles.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, further comprising the step of treating the patient with the TNFRSF agonist prior to the step of resecting of a tumor from the patient, wherein the TNFRSF agonist is administered intravenously at a dose of between 0.1 mg/kg and 50 mg/kg every four weeks for up to eight cycles.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein the TNFRSF agonist is selected from the group consisting of urelumab, utomilumab, EU-101, tavolixizumab, Creative Biolabs MOM-18455, and fragments, derivatives, variants, biosimilars, and combinations thereof.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein the first cell culture medium comprises a second TNFRSF agonist.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein the TNFRSF agonist is added to the first cell culture medium during the initial expansion at an interval selected from the group consisting of every day, every two days, every three days, every four days, every five days, every six days, every seven days, and every two weeks.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein the TNFRSF agonist is added to the second cell culture medium during the rapid expansion at an interval selected from the group consisting of every day, every two days, every three days, every four days, every five days, every six days, every seven days, and every two weeks.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein the TNFRSF agonist is added at a concentration sufficient to achieve a concentration in the cell culture medium of between 0.1 μg/mL and 100 μg/mL.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein the TNFRSF agonist is added at a concentration sufficient to achieve a concentration in the cell culture medium of between 20 μg/mL and 40 μg/mL.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein IL-2 is present at an initial concentration of about 10 to about 6000 IU/mL in the first cell culture medium.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein IL-2 is present at an initial concentration of about 3000 IU/mL in the first cell culture medium.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, The method of Claim 31, wherein IL-2 is present at an initial concentration of about 800 to about 1100 IU/mL in the first cell culture medium.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein IL-2 is present at an initial concentration of about 1000 IU/mL in the first cell culture medium.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein IL-2 is present at an initial concentration of about 10 to about 6000 IU/mL in the second cell culture medium.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein IL-2 is present at an initial concentration of about 3000 IU/mL in the second cell culture medium.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein IL-2 is present at an initial concentration of about 800 to about 1100 IU/mL in the second cell culture medium.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein IL-2 is present at an initial concentration of about 1000 IU/mL in the second cell culture medium.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein IL-15 is present in the first cell culture medium.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein IL-15 is present at an initial concentration of about 5 ng/mL to about 20 ng/mL in the first cell culture medium.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein IL-15 is present in the second cell culture medium.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein IL-15 is present at an initial concentration of about 5 ng/mL to about 20 ng/mL in the second cell culture medium.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein IL-21 is present in the first cell culture medium.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein IL-21 is present at an initial concentration of about 5 ng/mL to about 20 ng/mL in the first cell culture medium.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein IL-21 is present in the second cell culture medium.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein IL-21 is present at an initial concentration of about 5 ng/mL to about 20 ng/mL in the second cell culture medium.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein OKT-3 antibody is present at an initial concentration of about 10 ng/mL to about 60 ng/mL in the second cell culture medium.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein OKT-3 antibody is present at an initial concentration of about 30 ng/mL in the second cell culture medium.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein the initial expansion is performed using a gas permeable container.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein the rapid expansion is performed using a gas permeable container.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, further comprising the step of treating the patient with a non-myeloablative lymphodepletion regimen prior to administering the third population of TILs to the patient.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, further comprising the step of treating the patient with a non-myeloablative lymphodepletion regimen prior to administering the third population of TILs to the patient, wherein the non-myeloablative lymphodepletion regimen comprises the steps of administration of cyclophosphamide at a dose of 60 mg/m²/day for two days followed by administration of fludarabine at a dose of 25 mg/m²/day for five days.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, further comprising the step of treating the patient with a decrescendo IL-2 regimen starting on the day after administration of the third population of TILs to the patient, wherein the decrescendo IL-2 regimen comprises aldesleukin administered intravenously at a dose of 18,000,000 IU/m² on day 1, 9,000,000 IU/m² on day 2, and 4,500,000 IU/m² on days 3 and 4.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, further comprising the step of treating the patient with pegylated IL-2 after administration of the third population of TILs to the patient at a dose of 0.10 mg/day to 50 mg/day.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, further comprising the step of treating the patient with a high-dose IL-2 regimen starting on the day after administration of the third population of TILs to the patient.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, further comprising the step of treating the patient with a high-dose IL-2 regimen starting on the day after administration of the third population of TILs to the patient, wherein the high-dose IL-2 regimen comprises 600,000 or 720,000 IU/kg of aldesleukin, or a biosimilar or variant thereof, administered as a 15-minute bolus intravenous infusion every eight hours until tolerance.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein the cancer is selected from the group consisting of melanoma, ovarian cancer, cervical cancer, lung cancer, bladder cancer, breast cancer, head and neck cancer, renal cell carcinoma, acute myeloid leukemia, colorectal cancer, cholangiocarcinoma, and sarcoma.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, wherein the cancer is selected from the group consisting of non-small cell lung cancer (NSCLC), triple negative breast cancer, double-refractory melanoma, and uveal (ocular) melanoma.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, further comprising the step of treating the patient with a PD-1 inhibitor or PD-L1 inhibitor prior to resecting the tumor from the patient.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, further comprising the step of treating the patient with a PD-1 inhibitor or PD-L1 inhibitor prior to resecting the tumor from the patient, wherein the PD-1 inhibitor or PD-L1 inhibitor is selected from the group consisting of nivolumab, pembrolizumab, durvalumab, atezolizumab, avelumab, and fragments, derivatives, variants, biosimilars, and combinations thereof.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, further comprising the step of treating the patient with a PD-1 inhibitor or PD-L1 inhibitor after resecting the tumor from the patient.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, further comprising the step of treating the patient with a PD-1 inhibitor or PD-L1 inhibitor after resecting the tumor from the patient, wherein the PD-1 inhibitor or PD-L1 inhibitor is selected from the group consisting of nivolumab, pembrolizumab, durvalumab, atezolizumab, avelumab, and fragments, derivatives, variants, biosimilars, and combinations thereof.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, further comprising the step of treating the patient with a PD-1 inhibitor or PD-L1 inhibitor after administering the third population of TILs to the patient.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer, further comprising the step of treating the patient with a PD-1 inhibitor or PD-L1 inhibitor after administering the third population of TILs to the patient, wherein the PD-1 inhibitor or PD-L1 inhibitor is selected from the group consisting of nivolumab, pembrolizumab, durvalumab, atezolizumab, avelumab, and fragments, derivatives, variants, biosimilars, and combinations thereof.

In an embodiment, the invention provides a process for the preparation of a population of tumor infiltrating lymphocytes (TILs) comprising the steps of:
(b) obtaining a first population of TILs;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less; and
(e) harvesting the third population of TILs.

In an embodiment, the invention provides a population of tumor infiltrating lymphocytes (TILs) obtainable from a process comprising the steps of:
(b) obtaining a first population of TILs;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less; and
(e) harvesting the third population of TILs.

In an embodiment, the invention provides a population of TILs is for use in the treatment of cancer. In an embodiment, the invention provides a pharmaceutical composition comprising a population of tumor infiltrating lymphocytes (TILs) for use in treating a cancer wherein the population of tumor infiltrating lymphocytes (TILs) is obtainable by a process comprising the steps of:
(b) obtaining a first population of TILs;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less; and
(e) harvesting the third population of TILs.

In an embodiment, the first population of TILs is obtained from a tumor. In an embodiment, the tumor is firstly resected from a patient. In an embodiment, the first population of TILs is obtained from the tumor which has been resected from a patient. In an embodiment, the population of TILs is for administration in a therapeutically effective amount to a patient with cancer.

In an embodiment, the invention provides a method of expanding a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2, and wherein the initial expansion is performed over a period of 11 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3) antibody, peripheral blood mononuclear cells (PBMCs), and a TNFRSF agonist, and wherein the rapid expansion is performed over a period of 11 days or less;
(e) harvesting the third population of TILs; and
(f) optionally cryopreserving the third population of TILs in a dimethylsulfoxide-based media.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2, and wherein the initial expansion is performed over a period of 11 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3) antibody, peripheral blood mononuclear cells (PBMCs), and a TNFRSF agonist, and wherein the rapid expansion is performed over a period of 11 days or less;
(e) harvesting the third population of TILs; and
(f) administering a therapeutically effective portion of the third population of TILs to the patient.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2, and wherein the initial expansion is performed over a period of 11 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3) antibody, peripheral blood mononuclear cells (PBMCs), and a TNFRSF agonist, and wherein the rapid expansion is performed over a period of 11 days or less;
(e) harvesting the third population of TILs; and
(f) administering a therapeutically effective portion of the third population of TILs to the patient,
wherein the TNFRSF agonist is selected from the group consisting of a 4-1BB agonist, an OX40 agonist, and a combination thereof.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2, and wherein the initial expansion is performed over a period of 11 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3) antibody, peripheral blood mononuclear cells (PBMCs), and a TNFRSF agonist, and wherein the rapid expansion is performed over a period of 11 days or less;
(e) harvesting the third population of TILs; and
(f) administering a therapeutically effective portion of the third population of TILs to the patient,
wherein the TNFRSF agonist is selected from the group consisting of a 4-1BB agonist, an OX40 agonist, and a combination thereof, and
wherein the TNFRSF agonist is a 4-1BB agonist, and the 4-1BB agonist is selected from the group consisting of urelumab, utomilumab, EU-101, a fusion protein, and fragments, derivatives, variants, biosimilars, and combinations thereof.

In an embodiment, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2, and wherein the initial expansion is performed over a period of 11 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3) antibody, peripheral blood mononuclear cells (PBMCs), and a TNFRSF agonist, and wherein the rapid expansion is performed over a period of 11 days or less;
(e) harvesting the third population of TILs; and
(f) administering a therapeutically effective portion of the third population of TILs to the patient,
wherein the TNFRSF agonist is selected from the group consisting of a 4-1BB agonist, an OX40 agonist, and a combination thereof, and
wherein the TNFRSF agonist is a OX40 agonist, and the OX40 agonist is selected from the group consisting of tavolixizumab, GSK3174998, MEDI6469, MEDI6383, MOXR0916, PF-04518600, Creative Biolabs MOM-18455, and fragments, derivatives, variants, biosimilars, and combinations thereof,
wherein the OX4 agonist is present at the start of step (d) at a concentration between 1 μg/mL and 30 μg/mL.

In an embodiment, the invention provides a method of any of the foregoing embodiments, wherein the TNFRSF agonist is present at the start of step (d) at a concentration between 5 μg/mL and 20 μg/mL.

In an embodiment, the invention provides a method of any of the foregoing embodiments, wherein the TNFRSF agonist is present at the start of step (d) at a concentration of about 10 μg/mL.

In an embodiment, the invention provides a method of any of the foregoing embodiments, wherein the TNFRSF agonist is maintained throughout step (d) at a concentration between 1 μg/mL and 30 μg/mL.

In an embodiment, the invention provides a method of any of the foregoing embodiments, wherein the TNFRSF agonist is maintained throughout step (d) at a concentration between 5 μg/mL and 20 μg/mL.

In an embodiment, the invention provides a method of any of the foregoing embodiments, wherein the TNFRSF agonist is maintained throughout step (d) at a concentration of about 10 μg/mL.

In an embodiment, the invention provides a method of any of the foregoing embodiments, wherein the third population of TILs exhibits an increased ratio of CD8⁺ TILs to CD4⁺ TILs in comparison to the reference ratio of CD8⁺ TILs to CD4⁺ TILs in the second population of TILs. In an embodiment, the increased ratio is selected from the group consisting of at least 1% greater than the reference ratio, at least 2% greater than the reference ratio, at least 5% greater than the reference ratio, at least 10% greater than the reference ratio, at least 15% greater than the reference ratio, at least 20% greater than the reference ratio, at least 25% greater than the reference ratio, at least 30% greater than the reference ratio, at least 35% greater than the reference ratio, at least 40% greater than the reference ratio, at least 45% greater than the reference ratio, and at least 50% greater than the reference ratio. In an embodiment, the increased ratio is between 5% and 80% greater than the reference ratio. In an embodiment, the increased ratio is between 10% and 70% greater than the reference ratio. In an embodiment, the increased ratio is between 15% and 60% greater than the reference ratio. In an of the foregoing embodiments, the reference ratio is obtained from a third TIL population that is a responder to the TNFRSF agonist.

In an embodiment, the invention provides a method of any of the foregoing embodiments, wherein the cancer is selected from the group consisting of melanoma, uveal (ocular) melanoma, ovarian cancer, cervical cancer, lung cancer, bladder cancer, breast cancer, head and neck cancer (head and neck squamous cell cancer), renal cell carcinoma, colorectal cancer, pancreatic cancer, glioblastoma, cholangiocarcinoma, and sarcoma. In an embodiment, the invention provides a method of any of the foregoing embodiments, wherein the cancer is selected from the group consisting of cutaneous melanoma, uveal (ocular) melanoma, platinum-resistant ovarian cancer, pancreatic ductal adenocarcinoma, osteosarcoma, triple-negative breast cancer, and non-small-cell lung cancer.

In an embodiment, any of the foregoing embodiments may be combined with any of the following embodiments.

In an embodiment, the process is an in vitro or an ex vivo process.

In an embodiment, the TNFRSF agonist is selected from the group consisting of a 4-1BB agonist, an OX40 agonist, a CD27 agonist, a GITR agonist, a HVEM agonist, a CD95 agonist, and combinations thereof.

In an embodiment, the TNFRSF agonist is a 4-1BB agonist.

In an embodiment, the TNFRSF agonist is a 4-1BB agonist, and the 4-1BB agonist is selected from the group consisting of urelumab, utomilumab, EU-101 and fragments, derivatives, variants, biosimilars, and combinations thereof.

In an embodiment, the TNFRSF agonist is a 4-1BB agonist, and the 4-1BB agonist is a 4-1BB agonist fusion protein.

In an embodiment, the TNFRSF agonist is a 4-1BB agonist fusion protein, and the 4-1BB agonist fusion protein comprises (i) a first soluble 4-1BB binding domain, (ii) a first peptide linker, (iii) a second soluble 4-1BB binding domain, (iv) a second peptide linker, and (v) a third soluble 4-1BB binding domain, further comprising an additional domain at the N-terminal and/or C-terminal end, and wherein the additional domain comprises a Fc fragment domain and hinge domain, and wherein the fusion protein is a dimeric structure according to structure I-A or structure I-B.

In an embodiment, the TNFRSF agonist is a OX40 agonist.

In an embodiment, the TNFRSF agonist is a OX40 agonist, and the OX40 agonist is selected from the group consisting of tavolixizumab, GSK3174998, MEDI6469, MEDI6383, MOXR0916, PF-04518600, Creative Biolabs MOM-18455, and fragments, derivatives, variants, biosimilars, and combinations thereof.

In an embodiment, the TNFRSF agonist is an OX40 agonist, and the OX40 agonist is an OX40 agonist fusion protein.

In an embodiment, the TNFRSF agonist is an OX40 agonist fusion protein, and the OX40 agonist fusion protein comprises (i) a first soluble OX40 binding domain, (ii) a first peptide linker, (iii) a second soluble OX40 binding domain, (iv) a second peptide linker, and (v) a third soluble OX40 binding domain, further comprising an additional domain at the N-terminal and/or C-terminal end, and wherein the additional domain comprises a Fc fragment domain and hinge domain, and wherein the fusion protein is a dimeric structure according to structure I-A or structure I-B.

In an embodiment, the TNFRSF agonist is a CD27 agonist.

In an embodiment, the TNFRSF agonist is a CD27 agonist, and the CD27 agonist is varlilumab, or a fragment, derivative, variant, or biosimilar thereof.

In an embodiment, the TNFRSF agonist is a CD27 agonist, and wherein the CD27 agonist is an CD27 agonist fusion protein.

In an embodiment, the TNFRSF agonist is a CD27 agonist, and the CD27 agonist fusion protein comprises (i) a first soluble CD27 binding domain, (ii) a first peptide linker, (iii) a second soluble CD27 binding domain, (iv) a second peptide linker, and (v) a third soluble CD27 binding domain, further comprising an additional domain at the N-terminal and/or C-terminal end, and wherein the additional domain comprises a Fc fragment domain and hinge domain, and wherein the fusion protein is a dimeric structure according to structure I-A or structure I-B.

In an embodiment, the TNFRSF agonist is a GITR agonist.

In an embodiment, the TNFRSF agonist is a GITR agonist, and the GITR agonist is selected from the group consisting of TRX518, 6C8, 36E5, 3D6, 61G6, 6H6, 61F6, 1D8, 17F10, 35D8, 49A1, 9E5, 31H6, 2155, 698, 706, 827, 1649, 1718, 1D7, 33C9, 33F6, 34G4, 35B10, 41E11, 41G5, 42A11, 44C1, 45A8, 46E11, 48H12, 48H7, 49D9, 49E2, 48A9, 5H7, 7A10, 9H6, and fragments, derivatives, variants, biosimilars, and combinations thereof.

In an embodiment, the TNFRSF agonist is an GITR agonist, and the GITR agonist is a GITR agonist fusion protein.

In an embodiment, the TNFRSF agonist is a GITR agonist fusion protein, and the GITR agonist fusion protein comprises (i) a first soluble GITR binding domain, (ii) a first peptide linker, (iii) a second soluble GITR binding domain, (iv) a second peptide linker, and (v) a third soluble GITR binding domain, further comprising an additional domain at the N-terminal and/or C-terminal end, and wherein the additional domain comprises a Fc fragment domain and hinge domain, and wherein the fusion protein is a dimeric structure according to structure I-A or structure I-B.

In an embodiment, the TNFRSF agonist is a HVEM agonist.

In an embodiment, the TNFRSF agonist is an HVEM agonist, and the HVEM agonist is a HVEM agonist fusion protein.

In an embodiment, the TNFRSF agonist is a HVEM agonist fusion protein, and wherein the HVEM agonist fusion protein comprises (i) a first soluble HVEM binding domain, (ii) a first peptide linker, (iii) a second soluble HVEM binding domain, (iv) a second peptide linker, and (v) a third soluble HVEM binding domain, further comprising an additional domain at the N-terminal and/or C-terminal end, and wherein the additional domain comprises a Fc fragment domain and hinge domain, and wherein the fusion protein is a dimeric structure according to structure I-A or structure I-B.

In an embodiment, the TNFRSF agonist is selected from the group consisting of urelumab, utomilumab, EU-101, tavolixizumab, Creative Biolabs MOM-18455, and fragments, derivatives, variants, biosimilars, and combinations thereof.

In an embodiment, the first cell culture medium comprises a second TNFRSF agonist.

In an embodiment, the TNFRSF agonist is added to the first cell culture medium during the initial expansion at an interval selected from the group consisting of every day, every two days, every three days, every four days, every five days, every six days, every seven days, and every two weeks.

In an embodiment, the TNFRSF agonist is added to the second cell culture medium during the rapid expansion at an interval selected from the group consisting of every day, every two days, every three days, every four days, every five days, every six days, every seven days, and every two weeks.

In an embodiment, the TNFRSF agonist is added at a concentration sufficient to achieve a concentration in the cell culture medium of between 0.1 μg/mL and 100 μg/mL.

In an embodiment, the TNFRSF agonist is added at a concentration sufficient to achieve a concentration in the cell culture medium of between 20 μg/mL and 40 μg/mL.

Further details of the TNFRSF agonists are provided herein.

In an embodiment, IL-2 is present at an initial concentration of about 10 to about 6000 IU/mL in the first cell culture medium.

In an embodiment, IL-2 is present at an initial concentration of about 3000 IU/mL in the first cell culture medium.

In an embodiment, IL-2 is present at an initial concentration of about 800 to about 1100 IU/mL in the first cell culture medium.

In an embodiment, IL-2 is present at an initial concentration of about 1000 IU/mL in the first cell culture medium.

In an embodiment, IL-2 is present at an initial concentration of about 10 to about 6000 IU/mL in the second cell culture medium.

In an embodiment, IL-2 is present at an initial concentration of about 3000 IU/mL in the second cell culture medium.

In an embodiment, IL-2 is present at an initial concentration of about 800 to about 1100 IU/mL in the second cell culture medium.

In an embodiment, IL-2 is present at an initial concentration of about 1000 IU/mL in the second cell culture medium.

In an embodiment, IL-15 is present in the first cell culture medium.

In an embodiment, IL-15 is present at an initial concentration of about 5 ng/mL to about 20 ng/mL in the first cell culture medium.

In an embodiment, IL-15 is present in the second cell culture medium.

In an embodiment, IL-15 is present at an initial concentration of about 5 ng/mL to about 20 ng/mL in the second cell culture medium.

In an embodiment, IL-21 is present in the first cell culture medium.

In an embodiment, IL-21 is present at an initial concentration of about 5 ng/mL to about 20 ng/mL in the first cell culture medium.

In an embodiment, IL-21 is present in the second cell culture medium.

In an embodiment, IL-21 is present at an initial concentration of about 5 ng/mL to about 20 ng/mL in the second cell culture medium.

In an embodiment, OKT-3 antibody is present at an initial concentration of about 10 ng/mL to about 60 ng/mL in the second cell culture medium.

In an embodiment, OKT-3 antibody is present at an initial concentration of about 30 ng/mL in the second cell culture medium.

In an embodiment, the initial expansion is performed using a gas permeable container.

In an embodiment, the rapid expansion is performed using a gas permeable container.

In an embodiment, the invention provides a population of tumor infiltrating lymphocytes (TILs) for use in treating a cancer wherein the population of tumor infiltrating lymphocytes (TILs) is obtainable by a process of the invention as described herein.

In an embodiment, the invention provides a pharmaceutical composition comprising a population of tumor infiltrating lymphocytes (TILs) for use in a method of treating a cancer wherein the population of tumor infiltrating lymphocytes (TILs) is obtainable by a process of the invention as described herein.

In an embodiment, the population of TILs and/or the pharmaceutical composition is for use in treating cancer in combination with a TNFRSF.

In an embodiment, the invention provides a combination of a population of TILs obtainable by a process of the invention as described herein and a TNFRSF for use in the treatment of cancer.

In an embodiment, the population of TILs and/or the pharmaceutical composition is for use in treating cancer in combination with a TNFRSF agonist wherein the TNFRSF agonist is for administration on the day after administration of the third population of TILs to the patient, and wherein the TNFRSF agonist is administered intravenously at a dose of between 0.1 mg/kg and 50 mg/kg every four weeks for up to eight cycles.

In an embodiment, the population of TILs and/or the pharmaceutical composition is for use in treating cancer in combination with a TNFRSF agonist wherein the TNFRSF agonist is for administration prior to the step of resecting of a tumor from the patient, and wherein the TNFRSF agonist for administration intravenously at a dose of between 0.1 mg/kg and 50 mg/kg every four weeks for up to eight cycles.

In an embodiment, the population of TILs and/or the pharmaceutical composition is for use in treating cancer in combination with a non-myeloablative lymphodepletion regimen.

In an embodiment, the population of TILs and/or the pharmaceutical composition is for use in treating cancer in combination with a non-myeloablative lymphodepletion regimen prior to administering the third population of TILs and/or a pharmaceutical composition comprising the third population of TILs to the patient.

In an embodiment, the population of TILs and/or the pharmaceutical composition is for use in treating cancer in combination with a non-myeloablative lymphodepletion regimen prior to administering the third population of TILs and/or a pharmaceutical composition comprising the third population of TILs to the patient, wherein the non-myeloablative lymphodepletion regimen comprises the steps of administration of cyclophosphamide at a dose of 60 mg/m²/day for two days followed by administration of fludarabine at a dose of 25 mg/m²/day for five days. Further details of the non-myeloablative lymphodepletion regimen are provided herein, e.g., under the Heading “Non-Myeloablative Lymphodepletion with Chemotherapy”.

In an embodiment, the population of TILs and/or the pharmaceutical composition is for use in treating cancer in combination with a IL-2 regimen.

In an embodiment, the IL-2 regimen is a decrescendo IL-2 regimen.

In an embodiment, the population of TILs and/or the pharmaceutical composition is for use in treating cancer in combination with a decrescendo IL-2 regimen starting on the day after administration of the third population of TILs and/or a pharmaceutical composition comprising the third population of TILs to the patient, wherein the decrescendo IL-2 regimen comprises aldesleukin administered intravenously at a dose of 18,000,000 IU/m² on day 1, 9,000,000 IU/m² on day 2, and 4,500,000 IU/m² on days 3 and 4.

In an embodiment, the population of TILs and/or the pharmaceutical composition is for use in treating cancer in combination with pegylated IL-2.

In an embodiment, the population of TILs and/or the pharmaceutical composition is for use in a method of treating cancer in combination with pegylated IL-2 administered after administration of the third population of TILs and/or a pharmaceutical composition comprising the third population of TILs to the patient at a dose of 0.10 mg/day to 50 mg/day.

In an embodiment, the population of TILs and/or the pharmaceutical composition is for use in a method of treating cancer in combination with a high-dose IL-2 regimen.

In an embodiment, the population of TILs and/or the pharmaceutical composition is for use in a method of treating cancer in combination with a high-dose IL-2 regimen starting on the day after administration of the third population of TILs and/or a pharmaceutical composition comprising the third population of TILs to the patient.

In an embodiment, the population of TILs and/or the pharmaceutical composition is for use in treating cancer in combination with a high-dose IL-2 regimen starting on the day after administration of the third population of TILs and/or a pharmaceutical composition comprising the third population of TILs to the patient, wherein the high-dose IL-2 regimen comprises 600,000 or 720,000 IU/kg of aldesleukin, or a biosimilar or variant thereof, administered as a 15-minute bolus intravenous infusion every eight hours until tolerance.

In an embodiment, the population of TILs and/or the pharmaceutical composition is for use in treating cancer, wherein the cancer is selected from the group consisting of melanoma, ovarian cancer, cervical cancer, lung cancer, bladder cancer, breast cancer, head and neck cancer, renal cell carcinoma, acute myeloid leukemia, colorectal cancer, cholangiocarcinoma, and sarcoma.

In an embodiment, the population of TILs and/or the pharmaceutical composition is for use in treating cancer, wherein the cancer is selected from the group consisting of non-small cell lung cancer (NSCLC), triple negative breast cancer, double-refractory melanoma, and uveal (ocular) melanoma.

In an embodiment, the population of TILs and/or the pharmaceutical composition is for use in treating cancer in combination with a PD-1 inhibitor or PD-L1 inhibitor.

In an embodiment, the population of TILs and/or the pharmaceutical composition is for use in treating cancer in combination with a PD-1 inhibitor or PD-L1 inhibitor, wherein the PD-1 inhibitor or PD-L1 inhibitor is selected from the group consisting of nivolumab, pembrolizumab, durvalumab, atezolizumab, avelumab, and fragments, derivatives, variants, biosimilars, and combinations thereof.

In an embodiment, the population of TILs and/or the pharmaceutical composition is for use in treating cancer in combination with a PD-1 inhibitor or PD-L1 inhibitor, wherein the PD-1 inhibitor or PD-L1 inhibitor is for administration prior to resecting the tumor from the patient.

In an embodiment, the population of TILs and/or the pharmaceutical composition is for use in treating cancer in combination with a PD-1 inhibitor or PD-L1 inhibitor prior to resecting the tumor from the patient, wherein the PD-1 inhibitor or PD-L1 inhibitor is selected from the group consisting of nivolumab, pembrolizumab, durvalumab, atezolizumab, avelumab, and fragments, derivatives, variants, biosimilars, and combinations thereof.

In an embodiment, the population of TILs and/or the pharmaceutical composition is for use in method of treating cancer in combination with a PD-1 inhibitor or PD-L1 inhibitor.

In an embodiment, the population of TILs and/or the pharmaceutical composition is for use in treating cancer in combination with a PD-1 inhibitor or PD-L1 inhibitor, wherein the PD-1 inhibitor or PD-L1 inhibitor is selected from the group consisting of nivolumab, pembrolizumab, durvalumab, atezolizumab, avelumab, and fragments, derivatives, variants, biosimilars, and combinations thereof.

In an embodiment, the population of TILs and/or the pharmaceutical composition is for use in a method of treating cancer in combination with a PD-1 inhibitor or PD-L1 inhibitor after resecting the tumor from the patient.

In an embodiment, the population of TILs and/or the pharmaceutical composition is for use in treating cancer in combination with a PD-1 inhibitor or PD-L1 inhibitor after resecting the tumor from the patient, wherein the PD-1 inhibitor or PD-L1 inhibitor is selected from the group consisting of nivolumab, pembrolizumab, durvalumab, atezolizumab, avelumab, and fragments, derivatives, variants, biosimilars, and combinations thereof.

In an embodiment, the population of TILs and/or the pharmaceutical composition is for use in treating cancer in combination with a PD-1 inhibitor or PD-L1 inhibitor, wherein the PD-1 or PD-L1 inhibitor is for administration after administering the third population of TILs and/or a pharmaceutical composition comprising the third population of TILs to the patient.

In an embodiment, the population of TILs and/or the pharmaceutical composition is for use in treating cancer in combination with a PD-1 inhibitor or PD-L1 inhibitor which is for administration after administering the third population of TILs to the patient, wherein the PD-1 inhibitor or PD-L1 inhibitor is selected from the group consisting of nivolumab, pembrolizumab, durvalumab, atezolizumab, avelumab, and fragments, derivatives, variants, biosimilars, and combinations thereof. Further details of the PD-1 inhibitor and the PD-L1 inhibitor are described herein e.g. under the heading “Combinations with PD-1 and PD-L1 Inhibitors”. In some embodiments, the population of TILs and/or the pharmaceutical composition comprising a population of TILs further comprise one or more features as described herein, for example, under the headings “Pharmaceutical Compositions, Dosages, and Dosing Regimens for TILs” and “Pharmaceutical Compositions, Dosages, and Dosing Regimens for TNFRSF Agonists”.

### BRIEF DESCRIPTION OF THE DRAWINGS

The foregoing summary, as well as the following detailed description of the invention, will be better understood when read in conjunction with the appended drawings.

FIG. 1 illustrates a TIL expansion and treatment process. TNFRSF agonists of the present disclosure may be used in both the pre-REP stage (top half of figure) or REP stage (bottom half of figure) and may be added when IL-2 is added to each cell culture. Step 1 refers to the addition of 4 tumor fragments into 10 G-Rex 10 flasks. At step 2, approximately 40×10⁶ TILs or greater are obtained. At step 3, a split occurs into 36 G-Rex 100 flasks for REP. TILs are harvested by centrifugation at step 4. Fresh TIL product is obtained at step 5 after a total process time of approximate 43 days, at which point TILs may be infused into a patient.

FIG. 2 illustrates a treatment protocol for use with TILs expanded with TNFRSF agonists of the present disclosure. Surgery (and tumor resection) occurs at the start, and lymphodepletion chemo refers to non-myeloablative lymphodepletion with chemotherapy as described elsewhere herein. TNFRSF agonists of the present disclosure may also be used during therapy as described herein after administration of TILs.

FIG. 3 illustrates the results of an assay to determine if 4-1BB-Fc hybridoma 4B5 activates 4-1BB signaling on Jurkat cells expressing NF-kB using a green fluorescent protein (GFP) reporter in a dose dependent manner. “Secondary” refers to activation of a secondary antibody.

FIG. 4 illustrates the results of an assay to determine if 4-1BB-Fc hybridoma 1C4 activates 4-1BB signaling on Jurkat cells expressing NF-kB using a GFP reporter in a dose dependent manner. “Secondary” refers to activation of a secondary antibody.

FIG. 5 illustrates the results of an assay to determine if 4-1BB-Fc hybridoma 9B4 activates 4-1BB signaling on Jurkat cells expressing NF-kB using a GFP reporter in a dose dependent manner. “Secondary” refers to activation of a secondary antibody.

FIG. 6 illustrates the results of an assay to determine if 4-1BB-Fc hybridoma 1D7 activates 4-1BB signaling on Jurkat cells expressing NF-kB using a GFP reporter in a dose dependent manner. “Secondary” refers to activation of a secondary antibody.

FIG. 7 illustrates the results of an assay to determine if 4-1BB-Fc hybridoma 1D10 activates 4-1BB signaling on Jurkat cells expressing NF-kB using a GFP reporter in a dose dependent manner. “Secondary” refers to activation of a secondary antibody.

FIG. 8 illustrates the results of an assay to determine if 4-1BB-Fc hybridoma 3C2 activates 4-1BB signaling on Jurkat cells expressing NF-kB using a GFP reporter in a dose dependent manner. “Secondary” refers to activation of a secondary antibody.

FIG. 9 illustrates the results of an assay to determine if 4-1BB-Fc hybridoma 10D12 activates 4-1BB signaling on Jurkat cells expressing NF-kB using a GFP reporter in a dose dependent manner. “Secondary” refers to activation of a secondary antibody.

FIG. 10 illustrates the results of an assay to determine if 4-1BB-Fc hybridoma 8D2 activates 4-1BB signaling on Jurkat cells expressing NF-kB using a GFP reporter in a dose dependent manner. “Secondary” refers to activation of a secondary antibody.

FIG. 11 illustrates the results of an assay to determine if 4-1BB-Fc hybridoma 4G6 activates 4-1BB signaling on Jurkat cells expressing NF-kB using a GFP reporter in a dose dependent manner. “Secondary” refers to activation of a secondary antibody.

FIG. 12 illustrates the results of an assay to determine if 4-1BB-Fc hybridoma 8E3 activates 4-1BB signaling on Jurkat cells expressing NF-kB using a GFP reporter in a dose dependent manner. “Secondary” refers to activation of a secondary antibody.

FIG. 13 illustrates an exemplary TIL expansion and manufacturing protocol (Process 2A).

FIG. 14 illustrates exemplary method steps undertaken in Process 2A.

FIG. 15 illustrates an exemplary TIL expansion protocol.

FIG. 16 illustrates binding affinity for Creative Biolabs (CB) and BPS Biosciences (BPS) 4-1BB agonist antibodies as assessed by percentage of 4-1BB+ cells by flow cytometry. CB 4-1BB agonist exhibited the highest binding affinity.

FIG. 17 illustrates binding affinity for Creative Biolabs (CB) and BPS Biosciences (BPS) 4-1BB agonist antibodies as assessed by mean fluorescence intensity (MFI). CB 4-1BB agonist exhibited the highest binding affinity.

FIG. 18 illustrates the results of an assessment of NF-κB pathway activation of anti-4-1BB agonistic antibodies.

FIG. 19 illustrates binding affinity for Creative Biolabs OX40 agonist antibody as assessed by percentage of OX40⁺ cells by flow cytometry.

FIG. 20 illustrates binding affinity for Creative Biolabs OX40 agonist antibodies as assessed by mean fluorescence intensity (MFI).

FIG. 21 illustrates comparable binding affinity between Creative Biolabs anti-OX40 agonist antibody (at five concentrations shown) and a commercial anti-OX40 (clone Ber-ACT35) agonist. The first letter of each tumor designation indicates histology: C=cervical; H=head and neck (head and neck squamous cell carcinoma); L=lung; and M=melanoma.

FIG. 22 illustrates the results of an assessment of NF-κB pathway activation of anti-OX40 agonist antibody. OX40 reporter cells were treated with either anti-OX40 alone or Isotype control at the concentrations of 1, 2, 4, 8, and 16 μg/mL with or without PBMC feeder cells for 24 hours. The cells were lysed using One-Step Luciferase reagent, and luciferase activity was measured by luminometer.

FIG. 23 illustrates the experimental design for 4-1BB and OX40 agonist experiments during pre-REP.

FIG. 24 illustrates the tumor histologies used in the experimental design of FIG. 23.

FIG. 25 illustrates the data analysis strategy used to assess the impact of 4-1BB and anti-OX40 agonists used during pre-REP on TIL performance and properties.

FIG. 26 illustrates total cell count results for cell expansion using CB 4-1BB agonist (N=3). NT=not tested (control). The p value was >0.99.

FIG. 27 illustrates total cell count results for cell expansion using CB OX40 agonist (N=5). NT=not tested (control). The p value was 0.06.

FIG. 28 illustrates total cell count results for cell expansion using CB 4-1BB agonist and OX-40 agonist (N=2). NT=not tested (control).

FIG. 29 illustrates total CD8⁺ cell count results for cell expansion using CB 4-1BB agonist (N=3). The p value was 0.5.

FIG. 30 illustrates total CD8⁺ cell count results for cell expansion using CB OX40 agonist (N=5). The p value was 0.03.

FIG. 31 illustrates total CD8⁺ cell count results for cell expansion using CB 4-1BB agonist and OX-40 agonist (N=2). NT=not tested (control).

FIG. 32 illustrates total CD8⁺/CD4⁺ cell count ratio results for cell expansion using CB 4-1BB agonist (N=3). The p value was 0.2.

FIG. 33 illustrates total CD8⁺/CD4⁺ cell count ratio results for cell expansion using CB OX40 agonist (N=5). The p value was 0.12.

FIG. 34 illustrates total CD8⁺/CD4⁺ cell count ratio results for cell expansion using CB 4-1BB agonist and OX-40 agonist (N=2). NT=not tested (control).

FIG. 35 illustrates the experimental scheme for REP propagation of pre-REP TILs expanded in the presence of 4-1BB or OX40 agonists.

FIG. 36 illustrates fold expansion of TILs expanded in REP from pre-REP TILs expanded in the presence of CB 4-1BB agonist versus TILs not treated in the pre-REP (NT).

FIG. 37 illustrates fold expansion of TILs expanded in REP from pre-REP TILs expanded in the presence of CB OX40 agonist versus TILs not treated in the pre-REP (NT).

FIG. 38 illustrates fold expansion of TILs expanded in REP from pre-REP TILs expanded in the presence of CB 4-1BB agonist and CB OX40 agonist versus TILs not treated in the pre-REP (NT).

FIG. 39 illustrates the histologies of twenty-one TIL lines used for assessment of CB OX40 agonist during the REP phase.

FIG. 40 illustrates the experimental scheme for assessment of CB OX40 agonist during the REP phase.

FIG. 41 illustrates that the presence of an OX40 agonistic antibody preferentially expands CD8⁺ TIL during REP (shown as a percentage of CD3⁺CD4⁺ cells).

FIG. 42 illustrates that the presence of an OX40 agonistic antibody preferentially expands CD8⁺ TIL during REP (shown as a percentage of CD3⁺CD8⁺ cells).

FIG. 43 illustrates that in non-responder TIL lines, down-regulation of OX40 was not observed in CD4⁺ subset following anti-OX40 treatment.

FIG. 44 illustrates experimental details for CB OX40 agonist dose titration in non-responder and responder TIL lines.

FIG. 45 illustrates the results of CB OX40 agonist dose titration in responder TIL lines.

FIG. 46 illustrates the results of CB OX40 agonist dose titration in non-responder TIL lines.

FIG. 47 illustrates comparable TCRvb repertoire profiles for responder L4005.

FIG. 48 illustrates comparable TCRvb repertoire profiles for responder H3005.

FIG. 49 illustrates comparable TCRvb repertoire profiles for responder M1022.

FIG. 50 illustrates the structures I-A and I-B, the cylinders refer to individual polypeptide binding domains. Structures I-A and I-B comprise three linearly-linked TNFRSF binding domains derived from e.g., 4-1BBL or an antibody that binds 4-1BB, which fold to form a trivalent protein, which is then linked to a second trivalent protein through IgG1-Fc (including CH3 and CH2 domains) is then used to link two of the trivalent proteins together through disulfide bonds (small elongated ovals), stabilizing the structure and providing an agonists capable of bringing together the intracellular signaling domains of the six receptors and signaling proteins to form a signaling complex. The TNFRSF binding domains denoted as cylinders may be scFv domains comprising, e.g., a VH and a VL chain connected by a linker that may comprise hydrophilic residues and Gly and Ser sequences for flexibility, as well as Glu and Lys for solubility.

### BRIEF DESCRIPTION OF THE SEQUENCE LISTING

SEQ ID NO:1 is the amino acid sequence of the heavy chain of muromonab.

SEQ ID NO:2 is the amino acid sequence of the light chain of muromonab.

SEQ ID NO:3 is the amino acid sequence of a recombinant human IL-2 protein.

SEQ ID NO:4 is the amino acid sequence of aldesleukin.

SEQ ID NO:5 is the amino acid sequence of a recombinant human IL-4 protein.

SEQ ID NO:6 is the amino acid sequence of a recombinant human IL-7 protein.

SEQ ID NO:7 is the amino acid sequence of a recombinant human IL-15 protein.

SEQ ID NO:8 is the amino acid sequence of a recombinant human IL-21 protein.

SEQ ID NO:9 is the amino acid sequence of human 4-1BB.

SEQ ID NO:10 is the amino acid sequence of murine 4-1BB.

SEQ ID NO:11 is the heavy chain for the 4-1BB agonist monoclonal antibody utomilumab (PF-05082566).

SEQ ID NO:12 is the light chain for the 4-1BB agonist monoclonal antibody utomilumab (PF-05082566).

SEQ ID NO:13 is the heavy chain variable region (V_{H}) for the 4-1BB agonist monoclonal antibody utomilumab (PF-05082566).

SEQ ID NO:14 is the light chain variable region (V_{L}) for the 4-1BB agonist monoclonal antibody utomilumab (PF-05082566).

SEQ ID NO:15 is the heavy chain CDR1 for the 4-1BB agonist monoclonal antibody utomilumab (PF-05082566).

SEQ ID NO:16 is the heavy chain CDR2 for the 4-1BB agonist monoclonal antibody utomilumab (PF-05082566).

SEQ ID NO:17 is the heavy chain CDR3 for the 4-1BB agonist monoclonal antibody utomilumab (PF-05082566).

SEQ ID NO:18 is the light chain CDR1 for the 4-1BB agonist monoclonal antibody utomilumab (PF-05082566).

SEQ ID NO:19 is the light chain CDR2 for the 4-1BB agonist monoclonal antibody utomilumab (PF-05082566).

SEQ ID NO:20 is the light chain CDR3 for the 4-1BB agonist monoclonal antibody utomilumab (PF-05082566).

SEQ ID NO:21 is the heavy chain for the 4-1BB agonist monoclonal antibody urelumab (BMS-663513).

SEQ ID NO:22 is the light chain for the 4-1BB agonist monoclonal antibody urelumab (BMS-663513).

SEQ ID NO:23 is the heavy chain variable region (V_{H}) for the 4-1BB agonist monoclonal antibody urelumab (BMS-663513).

SEQ ID NO:24 is the light chain variable region (V_{L}) for the 4-1BB agonist monoclonal antibody urelumab (BMS-663513).

SEQ ID NO:25 is the heavy chain CDR1 for the 4-1BB agonist monoclonal antibody urelumab (BMS-663513).

SEQ ID NO:26 is the heavy chain CDR2 for the 4-1BB agonist monoclonal antibody urelumab (BMS-663513).

SEQ ID NO:27 is the heavy chain CDR3 for the 4-1BB agonist monoclonal antibody urelumab (BMS-663513).

SEQ ID NO:28 is the light chain CDR1 for the 4-1BB agonist monoclonal antibody urelumab (BMS-663513).

SEQ ID NO:29 is the light chain CDR2 for the 4-1BB agonist monoclonal antibody urelumab (BMS-663513).

SEQ ID NO:30 is the light chain CDR3 for the 4-1BB agonist monoclonal antibody urelumab (BMS-663513).

SEQ ID NO:31 is an Fc domain for a TNFRSF agonist fusion protein.

SEQ ID NO:32 is a linker for a TNFRSF agonist fusion protein.

SEQ ID NO:33 is a linker for a TNFRSF agonist fusion protein.

SEQ ID NO:34 is a linker for a TNFRSF agonist fusion protein.

SEQ ID NO:35 is a linker for a TNFRSF agonist fusion protein.

SEQ ID NO:36 is a linker for a TNFRSF agonist fusion protein.

SEQ ID NO:37 is a linker for a TNFRSF agonist fusion protein.

SEQ ID NO:38 is a linker for a TNFRSF agonist fusion protein.

SEQ ID NO:39 is a linker for a TNFRSF agonist fusion protein.

SEQ ID NO:40 is a linker for a TNFRSF agonist fusion protein.

SEQ ID NO:41 is a linker for a TNFRSF agonist fusion protein.

SEQ ID NO:42 is an Fc domain for a TNFRSF agonist fusion protein.

SEQ ID NO:43 is a linker for a TNFRSF agonist fusion protein.

SEQ ID NO:44 is a linker for a TNFRSF agonist fusion protein.

SEQ ID NO:45 is a linker for a TNFRSF agonist fusion protein.

SEQ ID NO:46 is a 4-1BB ligand (4-1BBL) amino acid sequence.

SEQ ID NO:47 is a soluble portion of 4-1BBL polypeptide.

SEQ ID NO:48 is a heavy chain variable region (V_{H}) for the 4-1BB agonist antibody 4B4-1-1 version 1.

SEQ ID NO:49 is a light chain variable region (V_{L}) for the 4-1BB agonist antibody 4B4-1-1 version 1.

SEQ ID NO:50 is a heavy chain variable region (V_{H}) for the 4-1BB agonist antibody 4B4-1-1 version 2.

SEQ ID NO:51 is a light chain variable region (V_{L}) for the 4-1BB agonist antibody 4B4-1-1 version 2.

SEQ ID NO:52 is a heavy chain variable region (V_{H}) for the 4-1BB agonist antibody H39E3-2.

SEQ ID NO:53 is a light chain variable region (V_{L}) for the 4-1BB agonist antibody H39E3-2.

SEQ ID NO:54 is the amino acid sequence of human OX40.

SEQ ID NO:55 is the amino acid sequence of murine OX40.

SEQ ID NO:56 is the heavy chain for the OX40 agonist monoclonal antibody tavolixizumab (MEDI-0562).

SEQ ID NO:57 is the light chain for the OX40 agonist monoclonal antibody tavolixizumab (MEDI-0562).

SEQ ID NO:58 is the heavy chain variable region (V_{H}) for the OX40 agonist monoclonal antibody tavolixizumab (MEDI-0562).

SEQ ID NO:59 is the light chain variable region (V_{L}) for the OX40 agonist monoclonal antibody tavolixizumab (MEDI-0562).

SEQ ID NO:60 is the heavy chain CDRl for the OX40 agonist monoclonal antibody tavolixizumab (MEDI-0562).

SEQ ID NO:61 is the heavy chain CDR2 for the OX40 agonist monoclonal antibody tavolixizumab (MEDI-0562).

SEQ ID NO:62 is the heavy chain CDR3 for the OX40 agonist monoclonal antibody tavolixizumab (MEDI-0562).

SEQ ID NO:63 is the light chain CDR1 for the OX40 agonist monoclonal antibody tavolixizumab (MEDI-0562).

SEQ ID NO:64 is the light chain CDR2 for the OX40 agonist monoclonal antibody tavolixizumab (MEDI-0562).

SEQ ID NO:65 is the light chain CDR3 for the OX40 agonist monoclonal antibody tavolixizumab (MEDI-0562).

SEQ ID NO:66 is the heavy chain for the OX40 agonist monoclonal antibody 11D4.

SEQ ID NO:67 is the light chain for the OX40 agonist monoclonal antibody 11D4.

SEQ ID NO:68 is the heavy chain variable region (V_{H}) for the OX40 agonist monoclonal antibody 11D4.

SEQ ID NO:69 is the light chain variable region (V_{L}) for the OX40 agonist monoclonal antibody 11D4.

SEQ ID NO:70 is the heavy chain CDRl for the OX40 agonist monoclonal antibody 11D4.

SEQ ID NO:71 is the heavy chain CDR2 for the OX40 agonist monoclonal antibody 11D4.

SEQ ID NO:72 is the heavy chain CDR3 for the OX40 agonist monoclonal antibody 11D4.

SEQ ID NO:73 is the light chain CDR1 for the OX40 agonist monoclonal antibody 11D4.

SEQ ID NO:74 is the light chain CDR2 for the OX40 agonist monoclonal antibody 11D4.

SEQ ID NO:75 is the light chain CDR3 for the OX40 agonist monoclonal antibody 11D4.

SEQ ID NO:76 is the heavy chain for the OX40 agonist monoclonal antibody 18D8.

SEQ ID NO:77 is the light chain for the OX40 agonist monoclonal antibody 18D8.

SEQ ID NO:78 is the heavy chain variable region (V_{H}) for the OX40 agonist monoclonal antibody 18D8.

SEQ ID NO:79 is the light chain variable region (V_{L}) for the OX40 agonist monoclonal antibody 18D8.

SEQ ID NO:80 is the heavy chain CDRl for the OX40 agonist monoclonal antibody 18D8.

SEQ ID NO:81 is the heavy chain CDR2 for the OX40 agonist monoclonal antibody 18D8.

SEQ ID NO:82 is the heavy chain CDR3 for the OX40 agonist monoclonal antibody 18D8.

SEQ ID NO:83 is the light chain CDR1 for the OX40 agonist monoclonal antibody 18D8.

SEQ ID NO:84 is the light chain CDR2 for the OX40 agonist monoclonal antibody 18D8.

SEQ ID NO:85 is the light chain CDR3 for the OX40 agonist monoclonal antibody 18D8.

SEQ ID NO:86 is the heavy chain variable region (V_{H}) for the OX40 agonist monoclonal antibody Hu119-122.

SEQ ID NO:87 is the light chain variable region (V_{L}) for the OX40 agonist monoclonal antibody Hu119-122.

SEQ ID NO:88 is the heavy chain CDRl for the OX40 agonist monoclonal antibody Hu119-122.

SEQ ID NO:89 is the heavy chain CDR2 for the OX40 agonist monoclonal antibody Hu119-122.

SEQ ID NO:90 is the heavy chain CDR3 for the OX40 agonist monoclonal antibody Hu119-122.

SEQ ID NO:91 is the light chain CDR1 for the OX40 agonist monoclonal antibody Hu119-122.

SEQ ID NO:92 is the light chain CDR2 for the OX40 agonist monoclonal antibody Hu119-122.

SEQ ID NO:93 is the light chain CDR3 for the OX40 agonist monoclonal antibody Hu119-122.

SEQ ID NO:94 is the heavy chain variable region (V_{H}) for the OX40 agonist monoclonal antibody Hu106-222.

SEQ ID NO:95 is the light chain variable region (V_{L}) for the OX40 agonist monoclonal antibody Hu106-222.

SEQ ID NO:96 is the heavy chain CDRl for the OX40 agonist monoclonal antibody Hu106-222.

SEQ ID NO:97 is the heavy chain CDR2 for the OX40 agonist monoclonal antibody Hu106-222.

SEQ ID NO:98 is the heavy chain CDR3 for the OX40 agonist monoclonal antibody Hu106-222.

SEQ ID NO:99 is the light chain CDR1 for the OX40 agonist monoclonal antibody Hu106-222.

SEQ ID NO:100 is the light chain CDR2 for the OX40 agonist monoclonal antibody Hu106-222.

SEQ ID NO:101 is the light chain CDR3 for the OX40 agonist monoclonal antibody Hu106-222.

SEQ ID NO:102 is an OX40 ligand (OX40L) amino acid sequence.

SEQ ID NO:103 is a soluble portion of OX40L polypeptide.

SEQ ID NO:104 is an alternative soluble portion of OX40L polypeptide.

SEQ ID NO:105 is the heavy chain variable region (V_{H}) for the OX40 agonist monoclonal antibody 008.

SEQ ID NO:106 is the light chain variable region (V_{L}) for the OX40 agonist monoclonal antibody 008.

SEQ ID NO:107 is the heavy chain variable region (V_{H}) for the OX40 agonist monoclonal antibody 011.

SEQ ID NO:108 is the light chain variable region (V_{L}) for the OX40 agonist monoclonal antibody 011.

SEQ ID NO:109 is the heavy chain variable region (V_{H}) for the OX40 agonist monoclonal antibody 021.

SEQ ID NO:110 is the light chain variable region (V_{L}) for the OX40 agonist monoclonal antibody 021.

SEQ ID NO:111 is the heavy chain variable region (V_{H}) for the OX40 agonist monoclonal antibody 023.

SEQ ID NO:112 is the light chain variable region (V_{L}) for the OX40 agonist monoclonal antibody 023.

SEQ ID NO:113 is the heavy chain variable region (V_{H}) for an OX40 agonist monoclonal antibody.

SEQ ID NO:114 is the light chain variable region (V_{L}) for an OX40 agonist monoclonal antibody.

SEQ ID NO:115 is the heavy chain variable region (V_{H}) for an OX40 agonist monoclonal antibody.

SEQ ID NO:116 is the light chain variable region (V_{L}) for an OX40 agonist monoclonal antibody.

SEQ ID NO:117 is the heavy chain variable region (V_{H}) for a humanized OX40 agonist monoclonal antibody.

SEQ ID NO:118 is the heavy chain variable region (V_{H}) for a humanized OX40 agonist monoclonal antibody.

SEQ ID NO:119 is the light chain variable region (V_{L}) for a humanized OX40 agonist monoclonal antibody.

SEQ ID NO:120 is the light chain variable region (V_{L}) for a humanized OX40 agonist monoclonal antibody.

SEQ ID NO:121 is the heavy chain variable region (V_{H}) for a humanized OX40 agonist monoclonal antibody.

SEQ ID NO:122 is the heavy chain variable region (V_{H}) for a humanized OX40 agonist monoclonal antibody.

SEQ ID NO:123 is the light chain variable region (V_{L}) for a humanized OX40 agonist monoclonal antibody.

SEQ ID NO:124 is the light chain variable region (V_{L}) for a humanized OX40 agonist monoclonal antibody.

SEQ ID NO:125 is the heavy chain variable region (V_{H}) for an OX40 agonist monoclonal antibody.

SEQ ID NO:126 is the light chain variable region (V_{L}) for an OX40 agonist monoclonal antibody.

SEQ ID NO:127 is the amino acid sequence of human CD27.

SEQ ID NO:128 is the amino acid sequence of macaque CD27.

SEQ ID NO:129 is the heavy chain for the CD27 agonist monoclonal antibody varlilumab (CDX-1127).

SEQ ID NO:130 is the light chain for the CD27 agonist monoclonal antibody varlilumab (CDX-1127).

SEQ ID NO:131 is the heavy chain variable region (V_{H}) for the CD27 agonist monoclonal antibody varlilumab (CDX-1127).

SEQ ID NO:132 is the light chain variable region (V_{L}) for the CD27 agonist monoclonal antibody varlilumab (CDX-1127).

SEQ ID NO:133 is the heavy chain CDR1 for the CD27 agonist monoclonal antibody varlilumab (CDX-1127).

SEQ ID NO:134 is the heavy chain CDR2 for the CD27 agonist monoclonal antibody varlilumab (CDX-1127).

SEQ ID NO:135 is the heavy chain CDR3 for the CD27 agonist monoclonal antibody varlilumab (CDX-1127).

SEQ ID NO:136 is the light chain CDR1 for the CD27 agonist monoclonal antibody varlilumab (CDX-1127).

SEQ ID NO:137 is the light chain CDR2 for the CD27 agonist monoclonal antibody varlilumab (CDX-1127).

SEQ ID NO:138 is the light chain CDR3 for the CD27 agonist monoclonal antibody varlilumab (CDX-1127).

SEQ ID NO:139 is an CD27 ligand (CD70) amino acid sequence.

SEQ ID NO:140 is a soluble portion of CD70 polypeptide.

SEQ ID NO:141 is an alternative soluble portion of CD70 polypeptide.

SEQ ID NO:142 is the amino acid sequence of human GITR (human tumor necrosis factor receptor superfamily member 18 (TNFRSF18) protein).

SEQ ID NO:143 is the amino acid sequence of murine GITR (murine tumor necrosis factor receptor superfamily member 18 (TNFRSF18) protein).

SEQ ID NO:144 is the amino acid sequence of the heavy chain variant HuN6C8 (glycosylated) of the 6C8 humanized GITR agonist monoclonal antibody, with an N (asparagine) in CDR2, corresponding to SEQ ID NO:60 in U.S. Pat. No. 7,812,135.

SEQ ID NO:145 is the amino acid sequence of the heavy chain variant HuN6C8 (aglycosylated) of the 6C8 humanized GITR agonist monoclonal antibody, with an N (asparagine) in CDR2, corresponding to SEQ ID NO:61 in U.S. Pat. No. 7,812,135.

SEQ ID NO:146 is the amino acid sequence of the heavy chain variant HuQ6C8 (glycosylated) of the 6C8 humanized GITR agonist monoclonal antibody, with an Q (glutamine) in CDR2, corresponding to SEQ ID NO:62 in U.S. Pat. No. 7,812,135.

SEQ ID NO:147 is the amino acid sequence of the heavy chain variant HuQ6C8 (aglycosylated) of the 6C8 humanized GITR agonist monoclonal antibody, with an Q (glutamine) in CDR2, corresponding to SEQ ID NO:63 in U.S. Pat. No. 7,812,135.

SEQ ID NO:148 is the amino acid sequence of the light chain of the 6C8 humanized GITR agonist monoclonal antibody, corresponding to SEQ ID NO:58 in U.S. Pat. No. 7,812,135.

SEQ ID NO:149 is the amino acid sequence of the leader sequence that may optionally be included with the amino acid sequences of SEQ ID NO:144, SEQ ID NO:145, SEQ ID NO:146, or SEQ ID NO:147 in GITR agonist monoclonal antibodies.

SEQ ID NO:150 is the amino acid sequence of the leader sequence that may optionally be included with the amino acid sequence of SEQ ID NO:148 in GITR agonist monoclonal antibodies.

SEQ ID NO:151 is the amino acid sequence of the heavy chain variable region of the 6C8 humanized GITR agonist monoclonal antibody, corresponding to SEQ ID NO:1 in U.S. Pat. No. 7,812,135.

SEQ ID NO:152 is the amino acid sequence of the heavy chain variable region of the 6C8 humanized GITR agonist monoclonal antibody, corresponding to SEQ ID NO:66 in U.S. Pat. No. 7,812,135.

SEQ ID NO:153 is the amino acid sequence of the light chain variable region of the 6C8 humanized GITR agonist monoclonal antibody, corresponding to SEQ ID NO:2 in U.S. Pat. No. 7,812,135.

SEQ ID NO:154 is the amino acid sequence of the heavy chain CDR1 region of the 6C8 humanized GITR agonist monoclonal antibody, corresponding to SEQ ID NO:3 in U.S. Pat. No. 7,812,135.

SEQ ID NO:155 is the amino acid sequence of the heavy chain CDR2 region of the 6C8 humanized GITR agonist monoclonal antibody, corresponding to SEQ ID NO:4 in U.S. Pat. No. 7,812,135.

SEQ ID NO:156 is the amino acid sequence of the heavy chain CDR2 region of the 6C8 humanized GITR agonist monoclonal antibody, corresponding to SEQ ID NO:19 in U.S. Pat. No. 7,812,135.

SEQ ID NO:157 is the amino acid sequence of the heavy chain CDR3 region of the 6C8 humanized GITR agonist monoclonal antibody, corresponding to SEQ ID NO:5 in U.S. Pat. No. 7,812,135.

SEQ ID NO:158 is the amino acid sequence of the heavy chain CDR1 region of the 6C8 humanized GITR agonist monoclonal antibody, corresponding to SEQ ID NO:6 in U.S. Pat. No. 7,812,135.

SEQ ID NO:159 is the amino acid sequence of the heavy chain CDR2 region of the 6C8 humanized GITR agonist monoclonal antibody, corresponding to SEQ ID NO:7 in U.S. Pat. No. 7,812,135.

SEQ ID NO:160 is the amino acid sequence of the heavy chain CDR3 region of the 6C8 humanized GITR agonist monoclonal antibody, corresponding to SEQ ID NO:8 in U.S. Pat. No. 7,812,135.

SEQ ID NO:161 is the amino acid sequence of the heavy chain variant HuN6C8 (glycosylated) of the 6C8 chimeric GITR agonist monoclonal antibody, with an N (asparagine) in CDR2, corresponding to SEQ ID NO:23 in U.S. Pat. No. 7,812,135.

SEQ ID NO:162 is the amino acid sequence of the heavy chain variant HuQ6C8 (aglycosylated) of the 6C8 chimeric GITR agonist monoclonal antibody, with an Q (glutamine) in CDR2, corresponding to SEQ ID NO:24 in U.S. Pat. No. 7,812,135.

SEQ ID NO:163 is the amino acid sequence of the light chain of the 6C8 chimeric GITR agonist monoclonal antibody, corresponding to SEQ ID NO:22 in U.S. Pat. No. 7,812,135.

SEQ ID NO:164 is the amino acid sequence of the GITR agonist 36E5 heavy chain variable region from U.S. Pat. No. 8,709,424.

SEQ ID NO:165 is the amino acid sequence of the GITR agonist 36E5 light chain variable region from U.S. Pat. No. 8,709,424.

SEQ ID NO:166 is the amino acid sequence of the GITR agonist 3D6 heavy chain variable region from U.S. Pat. No. 8,709,424.

SEQ ID NO:167 is the amino acid sequence of the GITR agonist 3D6 light chain variable region from U.S. Pat. No. 8,709,424.

SEQ ID NO:168 is the amino acid sequence of the GITR agonist 61G6 heavy chain variable region from U.S. Pat. No. 8,709,424.

SEQ ID NO:169 is the amino acid sequence of the GITR agonist 61G6 light chain variable region from U.S. Pat. No. 8,709,424.

SEQ ID NO:170 is the amino acid sequence of the GITR agonist 6H6 heavy chain variable region from U.S. Pat. No. 8,709,424.

SEQ ID NO:171 is the amino acid sequence of the GITR agonist 6H6 light chain variable region from U.S. Pat. No. 8,709,424.

SEQ ID NO:172 is the amino acid sequence of the GITR agonist 61F6 heavy chain variable region from U.S. Pat. No. 8,709,424.

SEQ ID NO:173 is the amino acid sequence of the GITR agonist 61F6 light chain variable region from U.S. Pat. No. 8,709,424.

SEQ ID NO:174 is the amino acid sequence of the GITR agonist 1D8 heavy chain variable region from U.S. Pat. No. 8,709,424.

SEQ ID NO:175 is the amino acid sequence of the GITR agonist 1D8 light chain variable region from U.S. Pat. No. 8,709,424.

SEQ ID NO:176 is the amino acid sequence of the GITR agonist 17F10 heavy chain variable region from U.S. Pat. No. 8,709,424.

SEQ ID NO:177 is the amino acid sequence of the GITR agonist 17F10 light chain variable region from U.S. Pat. No. 8,709,424.

SEQ ID NO:178 is the amino acid sequence of the GITR agonist 35D8 heavy chain variable region from U.S. Pat. No. 8,709,424.

SEQ ID NO:179 is the amino acid sequence of the GITR agonist 35D8 light chain variable region from U.S. Pat. No. 8,709,424.

SEQ ID NO:180 is the amino acid sequence of the GITR agonist 49A1 heavy chain variable region from U.S. Pat. No. 8,709,424.

SEQ ID NO:181 is the amino acid sequence of the GITR agonist 49A1 light chain variable region from U.S. Pat. No. 8,709,424.

SEQ ID NO:182 is the amino acid sequence of the GITR agonist 9E5 heavy chain variable region from U.S. Pat. No. 8,709,424.

SEQ ID NO:183 is the amino acid sequence of the GITR agonist 9E5 light chain variable region from U.S. Pat. No. 8,709,424.

SEQ ID NO:184 is the amino acid sequence of the GITR agonist 31H6 heavy chain variable region from U.S. Pat. No. 8,709,424.

SEQ ID NO:185 is the amino acid sequence of the GITR agonist 31H6 light chain variable region from U.S. Pat. No. 8,709,424.

SEQ ID NO:186 is the amino acid sequence of the humanized GITR agonist 36E5 heavy chain variable region from U.S. Pat. No. 8,709,424.

SEQ ID NO:187 is the amino acid sequence of the humanized GITR agonist 36E5 light chain variable region from U.S. Pat. No. 8,709,424.

SEQ ID NO:188 is the amino acid sequence of the humanized GITR agonist 3D6 heavy chain variable region from U.S. Pat. No. 8,709,424.

SEQ ID NO:189 is the amino acid sequence of the humanized GITR agonist 3D6 light chain variable region from U.S. Pat. No. 8,709,424.

SEQ ID NO:190 is the amino acid sequence of the humanized GITR agonist 61G6 heavy chain variable region from U.S. Pat. No. 8,709,424.

SEQ ID NO:191 is the amino acid sequence of the humanized GITR agonist 61G6 light chain variable region from U.S. Pat. No. 8,709,424.

SEQ ID NO:192 is the amino acid sequence of the humanized GITR agonist 6H6 heavy chain variable region from U.S. Pat. No. 8,709,424.

SEQ ID NO:193 is the amino acid sequence of the humanized GITR agonist 6H6 light chain variable region from U.S. Pat. No. 8,709,424.

SEQ ID NO:194 is the amino acid sequence of the humanized GITR agonist 61F6 heavy chain variable region from U.S. Pat. No. 8,709,424.

SEQ ID NO:195 is the amino acid sequence of the humanized GITR agonist 61F6 light chain variable region from U.S. Pat. No. 8,709,424.

SEQ ID NO:196 is the amino acid sequence of the humanized GITR agonist 1D8 heavy chain variable region from U.S. Pat. No. 8,709,424.

SEQ ID NO:197 is the amino acid sequence of the humanized GITR agonist 1D8 light chain variable region from U.S. Pat. No. 8,709,424.

SEQ ID NO:198 is the amino acid sequence of the humanized GITR agonist 17F10 heavy chain variable region from U.S. Pat. No. 8,709,424.

SEQ ID NO:199 is the amino acid sequence of the humanized GITR agonist 17F10 light chain variable region from U.S. Pat. No. 8,709,424.

SEQ ID NO:200 is the amino acid sequence of the humanized GITR agonist 35D8 heavy chain variable region from U.S. Pat. No. 8,709,424.

SEQ ID NO:201 is the amino acid sequence of the humanized GITR agonist 35D8 light chain variable region from U.S. Pat. No. 8,709,424.

SEQ ID NO:202 is the amino acid sequence of the humanized GITR agonist 49A1 heavy chain variable region from U.S. Pat. No. 8,709,424.

SEQ ID NO:203 is the amino acid sequence of the humanized GITR agonist 49A1 light chain variable region from U.S. Pat. No. 8,709,424.

SEQ ID NO:204 is the amino acid sequence of the humanized GITR agonist 9E5 heavy chain variable region from U.S. Pat. No. 8,709,424.

SEQ ID NO:205 is the amino acid sequence of the humanized GITR agonist 9E5 light chain variable region from U.S. Pat. No. 8,709,424.

SEQ ID NO:206 is the amino acid sequence of the humanized GITR agonist 31H6 heavy chain variable region from U.S. Pat. No. 8,709,424.

SEQ ID NO:207 is the amino acid sequence of the humanized GITR agonist 31H6 light chain variable region from U.S. Pat. No. 8,709,424.

SEQ ID NO:208 is the amino acid sequence of the GITR agonist 2155 variable heavy chain from U.S. Patent Application Publication No. US 2013/0108641 A1.

SEQ ID NO:209 is the amino acid sequence of the GITR agonist 2155 variable light chain from U.S. Patent Application Publication No. US 2013/0108641 A1.

SEQ ID NO:210 is the amino acid sequence of the GITR agonist 2155 humanized (HC1) heavy chain from U.S. Patent Application Publication No. US 2013/0108641 A1.

SEQ ID NO:211 is the amino acid sequence of the GITR agonist 2155 humanized (HC2) heavy chain from U.S. Patent Application Publication No. US 2013/0108641 A1.

SEQ ID NO:212 is the amino acid sequence of the GITR agonist 2155 humanized (HC3a) heavy chain from U.S. Patent Application Publication No. US 2013/0108641 A1.

SEQ ID NO:213 is the amino acid sequence of the humanized (HC3b) GITR agonist heavy chain from U.S. Patent Application Publication No. US 2013/0108641 A1.

SEQ ID NO:214 is the amino acid sequence of the humanized (HC4) GITR agonist heavy chain from U.S. Patent Application Publication No. US 2013/0108641 A1.

SEQ ID NO:215 is the amino acid sequence of the 2155 humanized (LC1) GITR agonist light chain from U.S. Patent Application Publication No. US 2013/0108641 A1.

SEQ ID NO:216 is the amino acid sequence of the 2155 humanized (LC2a) GITR agonist light chain from U.S. Patent Application Publication No. US 2013/0108641 A1.

SEQ ID NO:217 is the amino acid sequence of the 2155 humanized (LC2b) GITR agonist light chain from U.S. Patent Application Publication No. US 2013/0108641 A1.

SEQ ID NO:218 is the amino acid sequence of the 2155 humanized (LC3) GITR agonist light chain from U.S. Patent Application Publication No. US 2013/0108641 A1.

SEQ ID NO:219 is the amino acid sequence of the GITR agonist 698 variable heavy chain from U.S. Patent Application Publication No. US 2013/0108641 A1.

SEQ ID NO:220 is the amino acid sequence of the GITR agonist 698 variable light chain from U.S. Patent Application Publication No. US 2013/0108641 A1.

SEQ ID NO:221 is the amino acid sequence of the GITR agonist 706 variable heavy chain from U.S. Patent Application Publication No. US 2013/0108641 A1.

SEQ ID NO:222 is the amino acid sequence of the GITR agonist 706 variable light chain from U.S. Patent Application Publication No. US 2013/0108641 A1.

SEQ ID NO:223 is the amino acid sequence of the GITR agonist 827 variable heavy chain from U.S. Patent Application Publication No. US 2013/0108641 A1.

SEQ ID NO:224 is the amino acid sequence of the GITR agonist 827 variable light chain from U.S. Patent Application Publication No. US 2013/0108641 A1.

SEQ ID NO:225 is the amino acid sequence of the GITR agonist 1718 variable heavy chain from U.S. Patent Application Publication No. US 2013/0108641 A1.

SEQ ID NO:226 is the amino acid sequence of the GITR agonist 1718 variable light chain from U.S. Patent Application Publication No. US 2013/0108641 A1.

SEQ ID NO:227 is the amino acid sequence of the GITR agonist 2155 heavy chain CDR3 from U.S. Patent Application Publication No. US 2013/0108641 A1.

SEQ ID NO:228 is the amino acid sequence of the GITR agonist 2155 heavy chain CDR2 from U.S. Patent Application Publication No. US 2013/0108641 A1.

SEQ ID NO:229 is the amino acid sequence of the GITR agonist 2155 heavy chain CDR1 from U.S. Patent Application Publication No. US 2013/0108641 A1.

SEQ ID NO:230 is the amino acid sequence of the GITR agonist 2155 light chain CDR3 from U.S. Patent Application Publication No. US 2013/0108641 A1.

SEQ ID NO:231 is the amino acid sequence of the GITR agonist 2155 light chain CDR2 from U.S. Patent Application Publication No. US 2013/0108641 A1.

SEQ ID NO:232 is the amino acid sequence of the GITR agonist 2155 light chain CDR1 from U.S. Patent Application Publication No. US 2013/0108641 A1.

SEQ ID NO:233 is the amino acid sequence of the GITR agonists 698 and 706 heavy chain CDR3 from U.S. Patent Application Publication No. US 2013/0108641 A1.

SEQ ID NO:234 is the amino acid sequence of the GITR agonists 698 and 706 heavy chain CDR2 from U.S. Patent Application Publication No. US 2013/0108641 A1.

SEQ ID NO:235 is the amino acid sequence of the GITR agonists 698 and 706 heavy chain CDR1 from U.S. Patent Application Publication No. US 2013/0108641 A1.

SEQ ID NO:236 is the amino acid sequence of the GITR agonist 698 light chain CDR3 from U.S. Patent Application Publication No. US 2013/0108641 A1.

SEQ ID NO:237 is the amino acid sequence of the GITR agonists 698, 706, 827, and 1649 light chain CDR2 from U.S. Patent Application Publication No. US 2013/0108641 A1.

SEQ ID NO:238 is the amino acid sequence of the GITR agonists 698, 706, 827, and 1649 light chain CDR1 from U.S. Patent Application Publication No. US 2013/0108641 A1.

SEQ ID NO:239 is the amino acid sequence of the GITR agonists 706, 827, and 1649 light chain CDR3 from U.S. Patent Application Publication No. US 2013/0108641 A1.

SEQ ID NO:240 is the amino acid sequence of the GITR agonists 827 and 1649 heavy chain CDR3 from U.S. Patent Application Publication No. US 2013/0108641 A1.

SEQ ID NO:241 is the amino acid sequence of the GITR agonist 827 heavy chain CDR2 from U.S. Patent Application Publication No. US 2013/0108641 A1.

SEQ ID NO:242 is the amino acid sequence of the GITR agonist 1649 heavy chain CDR2 from U.S. Patent Application Publication No. US 2013/0108641 A1.

SEQ ID NO:243 is the amino acid sequence of the GITR agonist 1718 heavy chain CDR3 from U.S. Patent Application Publication No. US 2013/0108641 A1.

SEQ ID NO:244 is the amino acid sequence of the GITR agonist 1718 heavy chain CDR2 from U.S. Patent Application Publication No. US 2013/0108641 A1.

SEQ ID NO:245 is the amino acid sequence of the GITR agonist 1718 heavy chain CDR1 from U.S. Patent Application Publication No. US 2013/0108641 A1.

SEQ ID NO:246 is the amino acid sequence of the GITR agonist 1718 light chain CDR3 from U.S. Patent Application Publication No. US 2013/0108641 A1.

SEQ ID NO:247 is the amino acid sequence of the GITR agonist 1718 light chain CDR2 from U.S. Patent Application Publication No. US 2013/0108641 A1.

SEQ ID NO:248 is the amino acid sequence of the GITR agonist 1718 light chain CDR1 from U.S. Patent Application Publication No. US 2013/0108641 A1.

SEQ ID NO:249 is the amino acid sequence of the GITR agonists 827 and 1649 heavy chain CDR1 from U.S. Patent Application Publication No. US 2013/0108641 A1.

SEQ ID NO:250 is the amino acid sequence of the GITR agonist 1D7 heavy chain from U.S. Patent Application Publication No. US 2015/0064204 A1.

SEQ ID NO:251 is the amino acid sequence of the GITR agonist 1D7 light chain from U.S. Patent Application Publication No. US 2015/0064204 A1.

SEQ ID NO:252 is the amino acid sequence of the GITR agonist 1D7 variable heavy chain from U.S. Patent Application Publication No. US 2015/0064204 A1.

SEQ ID NO:253 is the amino acid sequence of the GITR agonist 1D7 variable light chain from U.S. Patent Application Publication No. US 2015/0064204 A1.

SEQ ID NO:254 is the amino acid sequence of the GITR agonist 1D7 heavy chain CDR1 from U.S. Patent Application Publication No. US 2015/0064204 A1.

SEQ ID NO:255 is the amino acid sequence of the GITR agonist 1D7 heavy chain CDR2 from U.S. Patent Application Publication No. US 2015/0064204 A1.

SEQ ID NO:256 is the amino acid sequence of the GITR agonist 1D7 heavy chain CDR3 from U.S. Patent Application Publication No. US 2015/0064204 A1.

SEQ ID NO:257 is the amino acid sequence of the GITR agonist 1D7 light chain CDR1 from U.S. Patent Application Publication No. US 2015/0064204 A1.

SEQ ID NO:258 is the amino acid sequence of the GITR agonist 1D7 light chain CDR2 from U.S. Patent Application Publication No. US 2015/0064204 A1.

SEQ ID NO:259 is the amino acid sequence of the GITR agonist 1D7 light chain CDR3 from U.S. Patent Application Publication No. US 2015/0064204 A1.

SEQ ID NO:260 is the amino acid sequence of the GITR agonist 33C9 heavy chain from U.S. Patent Application Publication No. US 2015/0064204 A1.

SEQ ID NO:261 is the amino acid sequence of the GITR agonist 33C9 light chain from U.S. Patent Application Publication No. US 2015/0064204 A1.

SEQ ID NO:262 is the amino acid sequence of the GITR agonist 33C9 variable heavy chain from U.S. Patent Application Publication No. US 2015/0064204 A1.

SEQ ID NO:263 is the amino acid sequence of the GITR agonist 33C9 variable light chain from U.S. Patent Application Publication No. US 2015/0064204 A1.

SEQ ID NO:264 is the amino acid sequence of the GITR agonist 33C9 heavy chain CDR1 from U.S. Patent Application Publication No. US 2015/0064204 A1.

SEQ ID NO:265 is the amino acid sequence of the GITR agonist 33C9 heavy chain CDR2 from U.S. Patent Application Publication No. US 2015/0064204 A1.

SEQ ID NO:266 is the amino acid sequence of the GITR agonist 33C9 heavy chain CDR3 from U.S. Patent Application Publication No. US 2015/0064204 A1.

SEQ ID NO:267 is the amino acid sequence of the GITR agonist 33C9 light chain CDR1 from U.S. Patent Application Publication No. US 2015/0064204 A1.

SEQ ID NO:268 is the amino acid sequence of the GITR agonist 33C9 light chain CDR2 from U.S. Patent Application Publication No. US 2015/0064204 A1.

SEQ ID NO:269 is the amino acid sequence of the GITR agonist 33C9 light chain CDR3 from U.S. Patent Application Publication No. US 2015/0064204 A1.

SEQ ID NO:270 is the amino acid sequence of the GITR agonist 33F6 heavy chain from U.S. Patent Application Publication No. US 2015/0064204 A1.

SEQ ID NO:271 is the amino acid sequence of the GITR agonist 33F6 light chain from U.S. Patent Application Publication No. US 2015/0064204 A1.

SEQ ID NO:272 is the amino acid sequence of the GITR agonist 33F6 variable heavy chain from U.S. Patent Application Publication No. US 2015/0064204 A1.

SEQ ID NO:273 is the amino acid sequence of the GITR agonist 33F6 variable light chain from U.S. Patent Application Publication No. US 2015/0064204 A1.

SEQ ID NO:274 is the amino acid sequence of the GITR agonist 33F6 heavy chain CDR1 from U.S. Patent Application Publication No. US 2015/0064204 A1.

SEQ ID NO:275 is the amino acid sequence of the GITR agonist 33F6 heavy chain CDR2 from U.S. Patent Application Publication No. US 2015/0064204 A1.

SEQ ID NO:276 is the amino acid sequence of the GITR agonist 33F6 heavy chain CDR3 from U.S. Patent Application Publication No. US 2015/0064204 A1.

SEQ ID NO:277 is the amino acid sequence of the GITR agonist 33F6 light chain CDR1 from U.S. Patent Application Publication No. US 2015/0064204 A1.

SEQ ID NO:278 is the amino acid sequence of the GITR agonist 33F6 light chain CDR2 from U.S. Patent Application Publication No. US 2015/0064204 A1.

SEQ ID NO:279 is the amino acid sequence of the GITR agonist 33F6 light chain CDR3 from U.S. Patent Application Publication No. US 2015/0064204 A1.

SEQ ID NO:280 is the amino acid sequence of the GITR agonist 34G4 heavy chain from U.S. Patent Application Publication No. US 2015/0064204 A1.

SEQ ID NO:281 is the amino acid sequence of the GITR agonist 34G4 light chain from U.S. Patent Application Publication No. US 2015/0064204 A1.

SEQ ID NO:282 is the amino acid sequence of the GITR agonist 34G4 variable heavy chain from U.S. Patent Application Publication No. US 2015/0064204 A1.

SEQ ID NO:283 is the amino acid sequence of the GITR agonist 34G4 variable light chain from U.S. Patent Application Publication No. US 2015/0064204 A1.

SEQ ID NO:284 is the amino acid sequence of the GITR agonist 34G4 heavy chain CDR1 from U.S. Patent Application Publication No. US 2015/0064204 A1.

SEQ ID NO:285 is the amino acid sequence of the GITR agonist 34G4 heavy chain CDR2 from U.S. Patent Application Publication No. US 2015/0064204 A1.

SEQ ID NO:286 is the amino acid sequence of the GITR agonist 34G4 heavy chain CDR3 from U.S. Patent Application Publication No. US 2015/0064204 A1.

SEQ ID NO:287 is the amino acid sequence of the GITR agonist 34G4 light chain CDR1 from U.S. Patent Application Publication No. US 2015/0064204 A1.

SEQ ID NO:288 is the amino acid sequence of the GITR agonist 34G4 light chain CDR2 from U.S. Patent Application Publication No. US 2015/0064204 A1.

SEQ ID NO:289 is the amino acid sequence of the GITR agonist 34G4 light chain CDR3 from U.S. Patent Application Publication No. US 2015/0064204 A1.

SEQ ID NO:290 is the amino acid sequence of the GITR agonist 35B10 heavy chain from U.S. Patent Application Publication No. US 2015/0064204 A1.

SEQ ID NO:291 is the amino acid sequence of the GITR agonist 35B10 light chain from U.S. Patent Application Publication No. US 2015/0064204 A1.

SEQ ID NO:292 is the amino acid sequence of the GITR agonist 35B10 variable heavy chain from U.S. Patent Application Publication No. US 2015/0064204 A1.

SEQ ID NO:293 is the amino acid sequence of the GITR agonist 35B10 variable light chain from U.S. Patent Application Publication No. US 2015/0064204 A1.

SEQ ID NO:294 is the amino acid sequence of the GITR agonist 35B10 heavy chain CDR1 from U.S. Patent Application Publication No. US 2015/0064204 A1.

SEQ ID NO:295 is the amino acid sequence of the GITR agonist 35B10 heavy chain CDR2 from U.S. Patent Application Publication No. US 2015/0064204 A1.

SEQ ID NO:296 is the amino acid sequence of the GITR agonist 35B10 heavy chain CDR3 from U.S. Patent Application Publication No. US 2015/0064204 A1.

SEQ ID NO:297 is the amino acid sequence of the GITR agonist 35B10 light chain CDR1 from U.S. Patent Application Publication No. US 2015/0064204 A1.

SEQ ID NO:298 is the amino acid sequence of the GITR agonist 35B10 light chain CDR2 from U.S. Patent Application Publication No. US 2015/0064204 A1.

SEQ ID NO:299 is the amino acid sequence of the GITR agonist 35B10 light chain CDR3 from U.S. Patent Application Publication No. US 2015/0064204 A1.

SEQ ID NO:300 is the amino acid sequence of the GITR agonist 41E11 heavy chain from U.S. Patent Application Publication No. US 2015/0064204 A1.

SEQ ID NO:301 is the amino acid sequence of the GITR agonist 41E11 light chain from U.S. Patent Application Publication No. US 2015/0064204 A1.

SEQ ID NO:302 is the amino acid sequence of the GITR agonist 41E11 variable heavy chain from U.S. Patent Application Publication No. US 2015/0064204 A1.

SEQ ID NO:303 is the amino acid sequence of the GITR agonist 41E11 variable light chain from U.S. Patent Application Publication No. US 2015/0064204 A1.

SEQ ID NO:304 is the amino acid sequence of the GITR agonist 41E11 heavy chain CDR1 from U.S. Patent Application Publication No. US 2015/0064204 A1.

SEQ ID NO:305 is the amino acid sequence of the GITR agonist 41E11 heavy chain CDR2 from U.S. Patent Application Publication No. US 2015/0064204 A1.

SEQ ID NO:306 is the amino acid sequence of the GITR agonist 41E11 heavy chain CDR3 from U.S. Patent Application Publication No. US 2015/0064204 A1.

SEQ ID NO:307 is the amino acid sequence of the GITR agonist 41E11 light chain CDR1 from U.S. Patent Application Publication No. US 2015/0064204 A1.

SEQ ID NO:308 is the amino acid sequence of the GITR agonist 41E11 light chain CDR2 from U.S. Patent Application Publication No. US 2015/0064204 A1.

SEQ ID NO:309 is the amino acid sequence of the GITR agonist 41E11 light chain CDR3 from U.S. Patent Application Publication No. US 2015/0064204 A1.

SEQ ID NO:310 is the amino acid sequence of the GITR agonist 41G5 heavy chain from U.S. Patent Application Publication No. US 2015/0064204 A1.

SEQ ID NO:311 is the amino acid sequence of the GITR agonist 41G5 light chain from U.S. Patent Application Publication No. US 2015/0064204 A1.

SEQ ID NO:312 is the amino acid sequence of the GITR agonist 41G5 variable heavy chain from U.S. Patent Application Publication No. US 2015/0064204 A1.

SEQ ID NO:313 is the amino acid sequence of the GITR agonist 41G5 variable light chain from U.S. Patent Application Publication No. US 2015/0064204 A1.

SEQ ID NO:314 is the amino acid sequence of the GITR agonist 41G5 heavy chain CDR1 from U.S. Patent Application Publication No. US 2015/0064204 A1.

SEQ ID NO:315 is the amino acid sequence of the GITR agonist 41G5 heavy chain CDR2 from U.S. Patent Application Publication No. US 2015/0064204 A1.

SEQ ID NO:316 is the amino acid sequence of the GITR agonist 41G5 heavy chain CDR3 from U.S. Patent Application Publication No. US 2015/0064204 A1.

SEQ ID NO:317 is the amino acid sequence of the GITR agonist 41G5 light chain CDR1 from U.S. Patent Application Publication No. US 2015/0064204 A1.

SEQ ID NO:318 is the amino acid sequence of the GITR agonist 41G5 light chain CDR2 from U.S. Patent Application Publication No. US 2015/0064204 A1.

SEQ ID NO:319 is the amino acid sequence of the GITR agonist 41G5 light chain CDR3 from U.S. Patent Application Publication No. US 2015/0064204 A1.

SEQ ID NO:320 is the amino acid sequence of the GITR agonist 42A11 heavy chain from U.S. Patent Application Publication No. US 2015/0064204 A1.

SEQ ID NO:321 is the amino acid sequence of the GITR agonist 42A11 light chain from U.S. Patent Application Publication No. US 2015/0064204 A1.

SEQ ID NO:322 is the amino acid sequence of the GITR agonist 42A11 variable heavy chain from U.S. Patent Application Publication No. US 2015/0064204 A1.

SEQ ID NO:323 is the amino acid sequence of the GITR agonist 42A11 variable light chain from U.S. Patent Application Publication No. US 2015/0064204 A1.

SEQ ID NO:324 is the amino acid sequence of the GITR agonist 42A11 heavy chain CDR1 from U.S. Patent Application Publication No. US 2015/0064204 A1.

SEQ ID NO:325 is the amino acid sequence of the GITR agonist 42A11 heavy chain CDR2 from U.S. Patent Application Publication No. US 2015/0064204 A1.

SEQ ID NO:326 is the amino acid sequence of the GITR agonist 42A11 heavy chain CDR3 from U.S. Patent Application Publication No. US 2015/0064204 A1.

SEQ ID NO:327 is the amino acid sequence of the GITR agonist 42A11 light chain CDR1 from U.S. Patent Application Publication No. US 2015/0064204 A1.

SEQ ID NO:328 is the amino acid sequence of the GITR agonist 42A11 light chain CDR2 from U.S. Patent Application Publication No. US 2015/0064204 A1.

SEQ ID NO:329 is the amino acid sequence of the GITR agonist 42A11 light chain CDR3 from U.S. Patent Application Publication No. US 2015/0064204 A1.

SEQ ID NO:330 is the amino acid sequence of the GITR agonist 44C1 heavy chain from U.S. Patent Application Publication No. US 2015/0064204 A1.

SEQ ID NO:331 is the amino acid sequence of the GITR agonist 44C1 light chain from U.S. Patent Application Publication No. US 2015/0064204 A1.

SEQ ID NO:332 is the amino acid sequence of the GITR agonist 44C1 variable heavy chain from U.S. Patent Application Publication No. US 2015/0064204 A1.

SEQ ID NO:333 is the amino acid sequence of the GITR agonist 44C1 variable light chain from U.S. Patent Application Publication No. US 2015/0064204 A1.

SEQ ID NO:334 is the amino acid sequence of the GITR agonist 44C1 heavy chain CDR1 from U.S. Patent Application Publication No. US 2015/0064204 A1.

SEQ ID NO:335 is the amino acid sequence of the GITR agonist 44C1 heavy chain CDR2 from U.S. Patent Application Publication No. US 2015/0064204 A1.

SEQ ID NO:336 is the amino acid sequence of the GITR agonist 44C1 heavy chain CDR3 from U.S. Patent Application Publication No. US 2015/0064204 A1.

SEQ ID NO:337 is the amino acid sequence of the GITR agonist 44C1 light chain CDR1 from U.S. Patent Application Publication No. US 2015/0064204 A1.

SEQ ID NO:338 is the amino acid sequence of the GITR agonist 44C1 light chain CDR2 from U.S. Patent Application Publication No. US 2015/0064204 A1.

SEQ ID NO:339 is the amino acid sequence of the GITR agonist 44C1 light chain CDR3 from U.S. Patent Application Publication No. US 2015/0064204 A1.

SEQ ID NO:340 is the amino acid sequence of the GITR agonist 45A8 heavy chain from U.S. Patent Application Publication No. US 2015/0064204 A1.

SEQ ID NO:341 is the amino acid sequence of the GITR agonist 45A8 light chain from U.S. Patent Application Publication No. US 2015/0064204 A1.

SEQ ID NO:342 is the amino acid sequence of the GITR agonist 45A8 variable heavy chain from U.S. Patent Application Publication No. US 2015/0064204 A1.

SEQ ID NO:343 is the amino acid sequence of the GITR agonist 45A8 variable light chain from U.S. Patent Application Publication No. US 2015/0064204 A1.

SEQ ID NO:344 is the amino acid sequence of the GITR agonist 45A8 heavy chain CDR1 from U.S. Patent Application Publication No. US 2015/0064204 A1.

SEQ ID NO:345 is the amino acid sequence of the GITR agonist 45A8 heavy chain CDR2 from U.S. Patent Application Publication No. US 2015/0064204 A1.

SEQ ID NO:346 is the amino acid sequence of the GITR agonist 45A8 heavy chain CDR3 from U.S. Patent Application Publication No. US 2015/0064204 A1.

SEQ ID NO:347 is the amino acid sequence of the GITR agonist 45A8 light chain CDR1 from U.S. Patent Application Publication No. US 2015/0064204 A1.

SEQ ID NO:348 is the amino acid sequence of the GITR agonist 45A8 light chain CDR2 from U.S. Patent Application Publication No. US 2015/0064204 A1.

SEQ ID NO:349 is the amino acid sequence of the GITR agonist 45A8 light chain CDR3 from U.S. Patent Application Publication No. US 2015/0064204 A1.

SEQ ID NO:350 is the amino acid sequence of the GITR agonist 46E11 heavy chain from U.S. Patent Application Publication No. US 2015/0064204 A1.

SEQ ID NO:351 is the amino acid sequence of the GITR agonist 46E11 light chain from U.S. Patent Application Publication No. US 2015/0064204 A1.

SEQ ID NO:352 is the amino acid sequence of the GITR agonist 46E11 variable heavy chain from U.S. Patent Application Publication No. US 2015/0064204 A1.

SEQ ID NO:353 is the amino acid sequence of the GITR agonist 46E11 variable light chain from U.S. Patent Application Publication No. US 2015/0064204 A1.

SEQ ID NO:354 is the amino acid sequence of the GITR agonist 46E11 heavy chain CDR1 from U.S. Patent Application Publication No. US 2015/0064204 A1.

SEQ ID NO:355 is the amino acid sequence of the GITR agonist 46E11 heavy chain CDR2 from U.S. Patent Application Publication No. US 2015/0064204 A1.

SEQ ID NO:356 is the amino acid sequence of the GITR agonist 46E11 heavy chain CDR3 from U.S. Patent Application Publication No. US 2015/0064204 A1.

SEQ ID NO:357 is the amino acid sequence of the GITR agonist 46E11 light chain CDR1 from U.S. Patent Application Publication No. US 2015/0064204 A1.

SEQ ID NO:358 is the amino acid sequence of the GITR agonist 46E11 light chain CDR2 from U.S. Patent Application Publication No. US 2015/0064204 A1.

SEQ ID NO:359 is the amino acid sequence of the GITR agonist 46E11 light chain CDR3 from U.S. Patent Application Publication No. US 2015/0064204 A1.

SEQ ID NO:360 is the amino acid sequence of the GITR agonist 48H12 heavy chain from U.S. Patent Application Publication No. US 2015/0064204 A1.

SEQ ID NO:361 is the amino acid sequence of the GITR agonist 48H12 light chain from U.S. Patent Application Publication No. US 2015/0064204 A1.

SEQ ID NO:362 is the amino acid sequence of the GITR agonist 48H12 variable heavy chain from U.S. Patent Application Publication No. US 2015/0064204 A1.

SEQ ID NO:363 is the amino acid sequence of the GITR agonist 48H12 variable light chain from U.S. Patent Application Publication No. US 2015/0064204 A1.

SEQ ID NO:364 is the amino acid sequence of the GITR agonist 48H12 heavy chain CDR1 from U.S. Patent Application Publication No. US 2015/0064204 A1.

SEQ ID NO:365 is the amino acid sequence of the GITR agonist 48H12 heavy chain CDR2 from U.S. Patent Application Publication No. US 2015/0064204 A1.

SEQ ID NO:366 is the amino acid sequence of the GITR agonist 48H12 heavy chain CDR3 from U.S. Patent Application Publication No. US 2015/0064204 A1.

SEQ ID NO:367 is the amino acid sequence of the GITR agonist 48H12 light chain CDR1 from U.S. Patent Application Publication No. US 2015/0064204 A1.

SEQ ID NO:368 is the amino acid sequence of the GITR agonist 48H12 light chain CDR2 from U.S. Patent Application Publication No. US 2015/0064204 A1.

SEQ ID NO:369 is the amino acid sequence of the GITR agonist 48H12 light chain CDR3 from U.S. Patent Application Publication No. US 2015/0064204 A1.

SEQ ID NO:370 is the amino acid sequence of the GITR agonist 48H7 heavy chain from U.S. Patent Application Publication No. US 2015/0064204 A1.

SEQ ID NO:371 is the amino acid sequence of the GITR agonist 48H7 light chain from U.S. Patent Application Publication No. US 2015/0064204 A1.

SEQ ID NO:372 is the amino acid sequence of the GITR agonist 48H7 variable heavy chain from U.S. Patent Application Publication No. US 2015/0064204 A1.

SEQ ID NO:373 is the amino acid sequence of the GITR agonist 48H7 variable light chain from U.S. Patent Application Publication No. US 2015/0064204 A1.

SEQ ID NO:374 is the amino acid sequence of the GITR agonist 48H7 heavy chain CDR1 from U.S. Patent Application Publication No. US 2015/0064204 A1.

SEQ ID NO:375 is the amino acid sequence of the GITR agonist 48H7 heavy chain CDR2 from U.S. Patent Application Publication No. US 2015/0064204 A1.

SEQ ID NO:376 is the amino acid sequence of the GITR agonist 48H7 heavy chain CDR3 from U.S. Patent Application Publication No. US 2015/0064204 A1.

SEQ ID NO:377 is the amino acid sequence of the GITR agonist 48H7 light chain CDR1 from U.S. Patent Application Publication No. US 2015/0064204 A1.

SEQ ID NO:378 is the amino acid sequence of the GITR agonist 48H7 light chain CDR2 from U.S. Patent Application Publication No. US 2015/0064204 A1.

SEQ ID NO:379 is the amino acid sequence of the GITR agonist 48H7 light chain CDR3 from U.S. Patent Application Publication No. US 2015/0064204 A1.

SEQ ID NO:380 is the amino acid sequence of the GITR agonist 49D9 heavy chain from U.S. Patent Application Publication No. US 2015/0064204 A1.

SEQ ID NO:381 is the amino acid sequence of the GITR agonist 49D9 light chain from U.S. Patent Application Publication No. US 2015/0064204 A1.

SEQ ID NO:382 is the amino acid sequence of the GITR agonist 49D9 variable heavy chain from U.S. Patent Application Publication No. US 2015/0064204 A1.

SEQ ID NO:383 is the amino acid sequence of the GITR agonist 49D9 variable light chain from U.S. Patent Application Publication No. US 2015/0064204 A1.

SEQ ID NO:384 is the amino acid sequence of the GITR agonist 49D9 heavy chain CDR1 from U.S. Patent Application Publication No. US 2015/0064204 A1.

SEQ ID NO:385 is the amino acid sequence of the GITR agonist 49D9 heavy chain CDR2 from U.S. Patent Application Publication No. US 2015/0064204 A1.

SEQ ID NO:386 is the amino acid sequence of the GITR agonist 49D9 heavy chain CDR3 from U.S. Patent Application Publication No. US 2015/0064204 A1.

SEQ ID NO:387 is the amino acid sequence of the GITR agonist 49D9 light chain CDR1 from U.S. Patent Application Publication No. US 2015/0064204 A1.

SEQ ID NO:388 is the amino acid sequence of the GITR agonist 49D9 light chain CDR2 from U.S. Patent Application Publication No. US 2015/0064204 A1.

SEQ ID NO:389 is the amino acid sequence of the GITR agonist 49D9 light chain CDR3 from U.S. Patent Application Publication No. US 2015/0064204 A1.

SEQ ID NO:390 is the amino acid sequence of the GITR agonist 49E2 heavy chain from U.S. Patent Application Publication No. US 2015/0064204 A1.

SEQ ID NO:391 is the amino acid sequence of the GITR agonist 49E2 light chain from U.S. Patent Application Publication No. US 2015/0064204 A1.

SEQ ID NO:392 is the amino acid sequence of the GITR agonist 49E2 variable heavy chain from U.S. Patent Application Publication No. US 2015/0064204 A1.

SEQ ID NO:393 is the amino acid sequence of the GITR agonist 49E2 variable light chain from U.S. Patent Application Publication No. US 2015/0064204 A1.

SEQ ID NO:394 is the amino acid sequence of the GITR agonist 49E2 heavy chain CDR1 from U.S. Patent Application Publication No. US 2015/0064204 A1.

SEQ ID NO:395 is the amino acid sequence of the GITR agonist 49E2 heavy chain CDR2 from U.S. Patent Application Publication No. US 2015/0064204 A1.

SEQ ID NO:396 is the amino acid sequence of the GITR agonist 49E2 heavy chain CDR3 from U.S. Patent Application Publication No. US 2015/0064204 A1.

SEQ ID NO:397 is the amino acid sequence of the GITR agonist 49E2 light chain CDR1 from U.S. Patent Application Publication No. US 2015/0064204 A1.

SEQ ID NO:398 is the amino acid sequence of the GITR agonist 49E2 light chain CDR2 from U.S. Patent Application Publication No. US 2015/0064204 A1.

SEQ ID NO:399 is the amino acid sequence of the GITR agonist 49E2 light chain CDR3 from U.S. Patent Application Publication No. US 2015/0064204 A1.

SEQ ID NO:400 is the amino acid sequence of the GITR agonist 48A9 heavy chain from U.S. Patent Application Publication No. US 2015/0064204 A1.

SEQ ID NO:401 is the amino acid sequence of the GITR agonist 48A9 light chain from U.S. Patent Application Publication No. US 2015/0064204 A1.

SEQ ID NO:402 is the amino acid sequence of the GITR agonist 48A9 variable heavy chain from U.S. Patent Application Publication No. US 2015/0064204 A1.

SEQ ID NO:403 is the amino acid sequence of the GITR agonist 48A9 variable light chain from U.S. Patent Application Publication No. US 2015/0064204 A1.

SEQ ID NO:404 is the amino acid sequence of the GITR agonist 48A9 heavy chain CDR1 from U.S. Patent Application Publication No. US 2015/0064204 A1.

SEQ ID NO:405 is the amino acid sequence of the GITR agonist 48A9 heavy chain CDR2 from U.S. Patent Application Publication No. US 2015/0064204 A1.

SEQ ID NO:406 is the amino acid sequence of the GITR agonist 48A9 heavy chain CDR3 from U.S. Patent Application Publication No. US 2015/0064204 A1.

SEQ ID NO:407 is the amino acid sequence of the GITR agonist 48A9 light chain CDR1 from U.S. Patent Application Publication No. US 2015/0064204 A1.

SEQ ID NO:408 is the amino acid sequence of the GITR agonist 48A9 light chain CDR2 from U.S. Patent Application Publication No. US 2015/0064204 A1.

SEQ ID NO:409 is the amino acid sequence of the GITR agonist 48A9 light chain CDR3 from U.S. Patent Application Publication No. US 2015/0064204 A1.

SEQ ID NO:410 is the amino acid sequence of the GITR agonist 5H7 heavy chain from U.S. Patent Application Publication No. US 2015/0064204 A1.

SEQ ID NO:411 is the amino acid sequence of the GITR agonist 5H7 light chain from U.S. Patent Application Publication No. US 2015/0064204 A1.

SEQ ID NO:412 is the amino acid sequence of the GITR agonist 5H7 variable heavy chain from U.S. Patent Application Publication No. US 2015/0064204 A1.

SEQ ID NO:413 is the amino acid sequence of the GITR agonist 5H7 variable light chain from U.S. Patent Application Publication No. US 2015/0064204 A1.

SEQ ID NO:414 is the amino acid sequence of the GITR agonist 5H7 heavy chain CDR1 from U.S. Patent Application Publication No. US 2015/0064204 A1.

SEQ ID NO:415 is the amino acid sequence of the GITR agonist 5H7 heavy chain CDR2 from U.S. Patent Application Publication No. US 2015/0064204 A1.

SEQ ID NO:416 is the amino acid sequence of the GITR agonist 5H7 heavy chain CDR3 from U.S. Patent Application Publication No. US 2015/0064204 A1.

SEQ ID NO:417 is the amino acid sequence of the GITR agonist 5H7 light chain CDR1 from U.S. Patent Application Publication No. US 2015/0064204 A1.

SEQ ID NO:418 is the amino acid sequence of the GITR agonist 5H7 light chain CDR2 from U.S. Patent Application Publication No. US 2015/0064204 A1.

SEQ ID NO:419 is the amino acid sequence of the GITR agonist 5H7 light chain CDR3 from U.S. Patent Application Publication No. US 2015/0064204 A1.

SEQ ID NO:420 is the amino acid sequence of the GITR agonist 7A10 heavy chain from U.S. Patent Application Publication No. US 2015/0064204 A1.

SEQ ID NO:421 is the amino acid sequence of the GITR agonist 7A10 light chain from U.S. Patent Application Publication No. US 2015/0064204 A1.

SEQ ID NO:422 is the amino acid sequence of the GITR agonist 7A10 variable heavy chain from U.S. Patent Application Publication No. US 2015/0064204 A1.

SEQ ID NO:423 is the amino acid sequence of the GITR agonist 7A10 variable light chain from U.S. Patent Application Publication No. US 2015/0064204 A1.

SEQ ID NO:424 is the amino acid sequence of the GITR agonist 7A10 heavy chain CDR1 from U.S. Patent Application Publication No. US 2015/0064204 A1.

SEQ ID NO:425 is the amino acid sequence of the GITR agonist 7A10 heavy chain CDR2 from U.S. Patent Application Publication No. US 2015/0064204 A1.

SEQ ID NO:426 is the amino acid sequence of the GITR agonist 7A10 heavy chain CDR3 from U.S. Patent Application Publication No. US 2015/0064204 A1.

SEQ ID NO:427 is the amino acid sequence of the GITR agonist 7A10 light chain CDR1 from U.S. Patent Application Publication No. US 2015/0064204 A1.

SEQ ID NO:428 is the amino acid sequence of the GITR agonist 7A10 light chain CDR2 from U.S. Patent Application Publication No. US 2015/0064204 A1.

SEQ ID NO:429 is the amino acid sequence of the GITR agonist 7A10 light chain CDR3 from U.S. Patent Application Publication No. US 2015/0064204 A1.

SEQ ID NO:430 is the amino acid sequence of the GITR agonist 9H6 heavy chain from U.S. Patent Application Publication No. US 2015/0064204 A1.

SEQ ID NO:431 is the amino acid sequence of the GITR agonist 9H6 light chain from U.S. Patent Application Publication No. US 2015/0064204 A1.

SEQ ID NO:432 is the amino acid sequence of the GITR agonist 9H6 variable heavy chain from U.S. Patent Application Publication No. US 2015/0064204 A1.

SEQ ID NO:433 is the amino acid sequence of the GITR agonist 9H6 variable light chain from U.S. Patent Application Publication No. US 2015/0064204 A1.

SEQ ID NO:434 is the amino acid sequence of the GITR agonist 9H6 heavy chain CDR1 from U.S. Patent Application Publication No. US 2015/0064204 A1.

SEQ ID NO:435 is the amino acid sequence of the GITR agonist 9H6 heavy chain CDR2 from U.S. Patent Application Publication No. US 2015/0064204 A1.

SEQ ID NO:436 is the amino acid sequence of the GITR agonist 9H6 heavy chain CDR3 from U.S. Patent Application Publication No. US 2015/0064204 A1.

SEQ ID NO:437 is the amino acid sequence of the GITR agonist 9H6 light chain CDR1 from U.S. Patent Application Publication No. US 2015/0064204 A1.

SEQ ID NO:438 is the amino acid sequence of the GITR agonist 9H6 light chain CDR2 from U.S. Patent Application Publication No. US 2015/0064204 A1.

SEQ ID NO:439 is the amino acid sequence of the GITR agonist 9H6 light chain CDR3 from U.S. Patent Application Publication No. US 2015/0064204 A1.

SEQ ID NO:440 is an GITR ligand (GITRL) amino acid sequence.

SEQ ID NO:441 is a soluble portion of GITRL polypeptide.

SEQ ID NO:442 is the amino acid sequence of human HVEM (CD270).

SEQ ID NO:443 is a HVEM ligand (LIGHT) amino acid sequence.

SEQ ID NO:444 is a soluble portion of LIGHT polypeptide.

SEQ ID NO:445 is an alternative soluble portion of LIGHT polypeptide.

SEQ ID NO:446 is an alternative soluble portion of LIGHT polypeptide.

SEQ ID NO:447 is the amino acid sequence of human CD95 isoform 1.

SEQ ID NO:448 is the amino acid sequence of human CD95 isoform 2.

SEQ ID NO:449 is the amino acid sequence of human CD95 isoform 3.

SEQ ID NO:450 is the amino acid sequence of human CD95 isoform 4.

SEQ ID NO:451 is the heavy chain variable region (V_{H}) for the CD95 agonist monoclonal antibody E09.

SEQ ID NO:452 is the light chain variable region (V_{L}) for the CD95 agonist monoclonal antibody E09.

SEQ ID NO:453 is the heavy chain CDRl for the CD95 agonist monoclonal antibody E09.

SEQ ID NO:454 is the heavy chain CDR2 for the CD95 agonist monoclonal antibody E09.

SEQ ID NO:455 is the heavy chain CDR3 for the CD95 agonist monoclonal antibody E09.

SEQ ID NO:456 is the light chain CDR1 for the CD95 agonist monoclonal antibody E09.

SEQ ID NO:457 is the light chain CDR2 for the CD95 agonist monoclonal antibody E09.

SEQ ID NO:458 is the light chain CDR3 for the CD95 agonist monoclonal antibody E09.

SEQ ID NO:459 is a CD95 ligand (CD95L) amino acid sequence.

SEQ ID NO:460 is a soluble portion of CD95L polypeptide.

SEQ ID NO:461 is an alternative soluble portion of CD95L polypeptide.

SEQ ID NO:462 is an alternative soluble portion of CD95L polypeptide.

SEQ ID NO:463 is the heavy chain amino acid sequence of the PD-1 inhibitor nivolumab.

SEQ ID NO:464 is the light chain amino acid sequence of the PD-1 inhibitor nivolumab.

SEQ ID NO:465 is the heavy chain variable region (V_{H}) amino acid sequence of the PD-1 inhibitor nivolumab.

SEQ ID NO:466 is the light chain variable region (V_{L}) amino acid sequence of the PD-1 inhibitor nivolumab.

SEQ ID NO:467 is the heavy chain CDR1 amino acid sequence of the PD-1 inhibitor nivolumab.

SEQ ID NO:468 is the heavy chain CDR2 amino acid sequence of the PD-1 inhibitor nivolumab.

SEQ ID NO:469 is the heavy chain CDR3 amino acid sequence of the PD-1 inhibitor nivolumab.

SEQ ID NO:470 is the light chain CDR1 amino acid sequence of the PD-1 inhibitor nivolumab.

SEQ ID NO:471 is the light chain CDR2 amino acid sequence of the PD-1 inhibitor nivolumab.

SEQ ID NO:472 is the light chain CDR3 amino acid sequence of the PD-1 inhibitor nivolumab.

SEQ ID NO:473 is the heavy chain amino acid sequence of the PD-1 inhibitor pembrolizumab.

SEQ ID NO:474 is the light chain amino acid sequence of the PD-1 inhibitor pembrolizumab.

SEQ ID NO:475 is the heavy chain variable region (V_{H}) amino acid sequence of the PD-1 inhibitor pembrolizumab.

SEQ ID NO:476 is the light chain variable region (V_{L}) amino acid sequence of the PD-1 inhibitor pembrolizumab.

SEQ ID NO:477 is the heavy chain CDR1 amino acid sequence of the PD-1 inhibitor pembrolizumab.

SEQ ID NO:478 is the heavy chain CDR2 amino acid sequence of the PD-1 inhibitor pembrolizumab.

SEQ ID NO:479 is the heavy chain CDR3 amino acid sequence of the PD-1 inhibitor pembrolizumab.

SEQ ID NO:480 is the light chain CDR1 amino acid sequence of the PD-1 inhibitor pembrolizumab.

SEQ ID NO:481 is the light chain CDR2 amino acid sequence of the PD-1 inhibitor pembrolizumab.

SEQ ID NO:482 is the light chain CDR3 amino acid sequence of the PD-1 inhibitor pembrolizumab.

SEQ ID NO:483 is the heavy chain amino acid sequence of the PD-L1 inhibitor durvalumab.

SEQ ID NO:484 is the light chain amino acid sequence of the PD-L1 inhibitor durvalumab.

SEQ ID NO:485 is the heavy chain variable region (V_{H}) amino acid sequence of the PD-L1 inhibitor durvalumab.

SEQ ID NO:486 is the light chain variable region (V_{L}) amino acid sequence of the PD-L1 inhibitor durvalumab.

SEQ ID NO:487 is the heavy chain CDR1 amino acid sequence of the PD-L1 inhibitor durvalumab.

SEQ ID NO:488 is the heavy chain CDR2 amino acid sequence of the PD-L1 inhibitor durvalumab.

SEQ ID NO:489 is the heavy chain CDR3 amino acid sequence of the PD-L1 inhibitor durvalumab.

SEQ ID NO:490 is the light chain CDR1 amino acid sequence of the PD-L1 inhibitor durvalumab.

SEQ ID NO:491 is the light chain CDR2 amino acid sequence of the PD-L1 inhibitor durvalumab.

SEQ ID NO:492 is the light chain CDR3 amino acid sequence of the PD-L1 inhibitor durvalumab.

SEQ ID NO:493 is the heavy chain amino acid sequence of the PD-L1 inhibitor avelumab.

SEQ ID NO:494 is the light chain amino acid sequence of the PD-L1 inhibitor avelumab.

SEQ ID NO:495 is the heavy chain variable region (V_{H}) amino acid sequence of the PD-L1 inhibitor avelumab.

SEQ ID NO:496 is the light chain variable region (V_{L}) amino acid sequence of the PD-L1 inhibitor avelumab.

SEQ ID NO:497 is the heavy chain CDR1 amino acid sequence of the PD-L1 inhibitor avelumab.

SEQ ID NO:498 is the heavy chain CDR2 amino acid sequence of the PD-L1 inhibitor avelumab.

SEQ ID NO:499 is the heavy chain CDR3 amino acid sequence of the PD-L1 inhibitor avelumab.

SEQ ID NO:500 is the light chain CDR1 amino acid sequence of the PD-L1 inhibitor avelumab.

SEQ ID NO:501 is the light chain CDR2 amino acid sequence of the PD-L1 inhibitor avelumab.

SEQ ID NO:502 is the light chain CDR3 amino acid sequence of the PD-L1 inhibitor avelumab.

SEQ ID NO:503 is the heavy chain amino acid sequence of the PD-L1 inhibitor atezolizumab.

SEQ ID NO:504 is the light chain amino acid sequence of the PD-L1 inhibitor atezolizumab.

SEQ ID NO:505 is the heavy chain variable region (V_{H}) amino acid sequence of the PD-L1 inhibitor atezolizumab.

SEQ ID NO:506 is the light chain variable region (V_{L}) amino acid sequence of the PD-L1 inhibitor atezolizumab.

SEQ ID NO:507 is the heavy chain CDR1 amino acid sequence of the PD-L1 inhibitor atezolizumab.

SEQ ID NO:508 is the heavy chain CDR2 amino acid sequence of the PD-L1 inhibitor atezolizumab.

SEQ ID NO:509 is the heavy chain CDR3 amino acid sequence of the PD-L1 inhibitor atezolizumab.

SEQ ID NO:510 is the light chain CDR1 amino acid sequence of the PD-L1 inhibitor atezolizumab.

SEQ ID NO:511 is the light chain CDR2 amino acid sequence of the PD-L1 inhibitor atezolizumab.

SEQ ID NO:512 is the light chain CDR3 amino acid sequence of the PD-L1 inhibitor atezolizumab.

### DETAILED DESCRIPTION OF THE INVENTION

Unless defined otherwise, all technical and scientific terms used herein have the same meaning as is commonly understood by one of skill in the art to which this invention belongs. All patents and publications referred to herein are incorporated by reference in their entireties.

### Definitions

The terms “co-administration,” “co-administering,” “administered in combination with,” “administering in combination with,” “simultaneous,” and “concurrent,” as used herein, encompass administration of two or more active pharmaceutical ingredients (in a preferred embodiment of the present invention, for example, at least one TNFRSF agonist and a plurality of TILs) to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The term “rapid expansion” means an increase in the number of antigen-specific TILs of at least about 3-fold (or 4-, 5-, 6-, 7-, 8-, or 9-fold) over a period of a week, more preferably at least about 10-fold (or 20-, 30-, 40-, 50-, 60-, 70-, 80-, or 90-fold) over a period of a week, or most preferably at least about 100-fold over a period of a week. A number of rapid expansion protocols are described herein.

By “tumor infiltrating lymphocytes” or “TILs” herein is meant a population of cells originally obtained as white blood cells that have left the bloodstream of a subject and migrated into a tumor. TILs include, but are not limited to, CD8⁺ cytotoxic T cells (lymphocytes), Th1 and Th17 CD4⁺ T cells, natural killer cells, dendritic cells and M1 macrophages. TILs include both primary and secondary TILs. “Primary TILs” are those that are obtained from patient tissue samples as outlined herein (sometimes referred to as “freshly harvested”), and “secondary TILs” are any TIL cell populations that have been expanded or proliferated as discussed herein, including, but not limited to bulk TILs and expanded TILs (“REP TILs” or “post-REP TILs”).

By “population of cells” (including TILs) herein is meant a number of cells that share common traits. In general, populations generally range from 1×10⁶ to 1×10¹⁰ in number, with different TIL populations comprising different numbers. For example, initial growth of primary TILs in the presence of IL-2 results in a population of bulk TILs of roughly 1×10⁸ cells. REP expansion is generally done to provide populations of 1.5×10⁹ to 1.5×10¹⁰ cells for infusion.

The term “central memory T cell” refers to a subset of T cells that in the human are CD45R0+ and constitutively express CCR7 (CCR7^{hi}) and CD62L (CD62^{hi}). The surface phenotype of central memory T cells also includes TCR, CD3, CD127 (IL-7R), and IL-15R. Transcription factors for central memory T cells include BCL-6, BCL-6B, MBD2, and BMI1. Central memory T cells primarily secret IL-2 and CD40L as effector molecules after TCR triggering. Central memory T cells are predominant in the CD4 compartment in blood, and in the human are proportionally enriched in lymph nodes and tonsils.

The term “anti-CD3 antibody” refers to an antibody or variant thereof, e.g., a monoclonal antibody and including human, humanized, chimeric or murine antibodies which are directed against the CD3 receptor in the T cell antigen receptor of mature T cells. Anti-CD3 antibodies include OKT-3, also known as muromonab. Anti-CD3 antibodies also include the UHCT1 clone, also known as T3 and CD3ε. Other anti-CD3 antibodies include, for example, otelixizumab, teplizumab, and visilizumab.

The term “OKT-3” (also referred to herein as “OKT3”) refers to a monoclonal antibody or biosimilar or variant thereof, including human, humanized, chimeric, or murine antibodies, directed against the CD3 receptor in the T cell antigen receptor of mature T cells, and includes commercially-available forms such as OKT-3 (30 ng/mL, MACS GMP CD3 pure, Miltenyi Biotech, Inc., San Diego, CA, USA) and muromonab or variants, conservative amino acid substitutions, glycoforms, or biosimilars thereof. The amino acid sequences of the heavy and light chains of muromonab are given in Table 1 (SEQ ID NO:1 and SEQ ID NO:2).

TABLE 1
Amino acid sequences of muromonab.

| Identifier | Sequence (One-Letter Amino Acid Symbols) |
|---|---|
| SEQ ID | QVQLQQSGAE LARPGASVKM SCKASGYTFT RYTMHWVKQR PGQGLEWIGY INPSRGYTNY 60 |
| NO: 1 | NQKFKDKATL TTDKSSSTAY MQLSSLTSED SAVYYCARYY DDHYCLDYWG QGTTLTVSSA 120 |
| Muromonab | KTTAPSVYPL APVCGGTTGS SVTLGCLVKG YFPEPVTLTW NSGSLSSGVH TFPAVLQSDL 180 |
| heavy | YTLSSSVTVT SSTWPSQSIT CNVAHPASST KVDKKIEPRP KSCDKTHTCP PCPAPELLGG 240 |
| chain | PSVFLFPPKP KDTLMISRTP EVTCVVVDVS HEDPEVKFNW YVDGVEVHNA KTKPREEQYN 300 |
| | STYRVVSVLT VLHQDWLNGK EYKCKVSNKA LPAPIEKTIS KAKGQPREPQ VYTLPPSRDE 360 |
| | LTKNQVSLTC LVKGFYPSDI AVEWESNGQP ENNYKTTPPV LDSDGSFFLY SKLTVDKSRW 420 |
| | QQGNVFSCSV MHEALHNHYT QKSLSLSPGK 450 |
| SEQ ID | QIVLTQSPAI MSASPGEKVT MTCSASSSVS YMNWYQQKSG TSPKRWIYDT SKLASGVPAH 60 |
| NO: 2 | FRGSGSGTSY SLTISGMEAE DAATYYCQQW SSNPFTFGSG TKLEINRADT APTVSIFPPS 120 |
| Muromonab | SEQLTSGGAS VVCFLNNFYP KDINVKWKID GSERQNGVLN SWTDQDSKDS TYSMSSTLTL 180 |
| light | TKDEYERHNS YTCEATHKTS TSPIVKSFNR NEC 213 |
| chain | |

The term “IL-2” (also referred to herein as “IL2”) refers to the T cell growth factor known as interleukin-2, and includes all forms of IL-2 including human and mammalian forms, conservative amino acid substitutions, glycoforms, biosimilars, and variants thereof. IL-2 is described, e.g., in Nelson, J. Immunol. 2004, 172, 3983-88 and Malek, Annu. Rev. Immunol. 2008, 26, 453-79, the disclosures of which are incorporated by reference herein. The amino acid sequence of recombinant human IL-2 suitable for use in the invention is given in Table 2 (SEQ ID NO:3). For example, the term IL-2 encompasses human, recombinant forms of IL-2 such as aldesleukin (PROLEUKIN, available commercially from multiple suppliers in 22 million IU per single use vials), as well as the form of recombinant IL-2 commercially supplied by CellGenix, Inc., Portsmouth, NH, USA (CELLGRO GMP) or ProSpec-Tany TechnoGene Ltd., East Brunswick, NJ, USA (Cat. No. CYT-209-b) and other commercial equivalents from other vendors. Aldesleukin (des-alanyl-1, serine-125 human IL-2) is a nonglycosylated human recombinant form of IL-2 with a molecular weight of approximately 15 kDa. The amino acid sequence of aldesleukin suitable for use in the invention is given in Table 2 (SEQ ID NO:4). The term IL-2 also encompasses pegylated forms of IL-2, as described herein, including the pegylated IL2 prodrug NKTR-214, available from Nektar Therapeutics, South San Francisco, CA, USA. NKTR-214 and pegylated IL-2 suitable for use in the invention is described in U.S. Patent Application Publication No. US 2014/0328791 A1 and International Patent Application Publication No. WO 2012/065086 A1, the disclosures of which are incorporated by reference herein. Alternative forms of conjugated IL-2 suitable for use in the invention are described in U.S. Pat. Nos. 4,766,106, 5,206,344, 5,089,261 and 4,902,502, the disclosures of which are incorporated by reference herein. Formulations of IL-2 suitable for use in the invention are described in U.S. Pat. No. 6,706,289, the disclosure of which is incorporated by reference herein.

TABLE 2
Amino acid sequences of interleukins.

| Identifier | Sequence (One-Letter Amino Acid Symbols) |
|---|---|
| SEQ ID NO: 3 | MAPTSSSTKK TQLQLEHLLL DLQMILNGIN NYKNPKLTRM LTFKFYMPKK ATELKHLQCL 60 |
| recombinant | EEELKPLEEV LNLAQSKNFH LRPRDLISNI NVIVLELKGS ETTFMCEYAD ETATIVEFLN 120 |
| human IL-2 | RWITFCQSII STLT 134 |
| (rhIL-2) | |
| SEQ ID NO: 4 | PTSSSTKKTQ LQLEHLLLDL QMILNGINNY KNPKLTRMLT FKFYMPKKAT ELKHLQCLEE 60 |
| Aldesleukin | ELKPLEEVLN LAQSKNFHLR PRDLISNINV IVLELKGSET TFMCEYADET ATIVEFLNRW 120 |
| | ITFSQSIIST LT 132 |
| SEQ ID NO: 5 | MHKCDITLQE IIKTLNSLTE QKTLCTELTV TDIFAASKNT TEKETFCRAA TVLRQFYSHH 60 |
| recombinant | EKDTRCLGAT AQQFHRHKQL IRFLKRLDRN LWGLAGLNSC PVKEANQSTL ENFLERLKTI 120 |
| human IL-4 | MREKYSKCSS 130 |
| (rhIL-4) | |
| SEQ ID NO: 6 | MDCDIEGKDG KQYESVLMVS IDQLLDSMKE IGSNCLNNEF NFFKRHICDA NKEGMFLFRA 60 |
| recombinant | ARKLRQFLKM NSTGDFDLHL LKVSEGTTIL LNCTGQVKGR KPAALGEAQP TKSLEENKSL 120 |
| human IL-7 | KEQKKLNDLC FLKRLLQEIK TCWNKILMGT KEH 153 |
| (rhIL-7) | |
| SEQ ID NO: 7 | MNWVNVISDL KKIEDLIQSM HIDATLYTES DVHPSCKVTA MKCFLLELQV ISLESGDASI 60 |
| recombinant | HDTVENLIIL ANNSLSSNGN VTESGCKECE ELEEKNIKEF LQSFVHIVQM FINTS 115 |
| human IL-15 | |
| (rhIL-15) | |
| SEQ ID NO: 8 | MQDRHMIRMR QLIDIVDQLK NYVNDLVPEF LPAPEDVETN CEWSAFSCFQ KAQLKSANTG 60 |
| recombinant | NNERIINVSI KKLKRKPPST NAGRRQKHRL TCPSCDSYEK KPPKEFLERF KSLLQKMIHQ 120 |
| human IL-21 | HLSSRTHGSE DS 132 |
| (rhIL-21) | |

The term “IL-4” (also referred to herein as “IL4”) refers to the cytokine known as interleukin 4, which is produced by Th2 T cells and by eosinophils, basophils, and mast cells. IL-4 regulates the differentiation of naïve helper T cells (Th0 cells) to Th2 T cells. Steinke and Borish, Respir. Res. 2001, 2, 66-70. Upon activation by IL-4, Th2 T cells subsequently produce additional IL-4 in a positive feedback loop. IL-4 also stimulates B cell proliferation and class II MHC expression, and induces class switching to IgE and IgG1 expression from B cells. Recombinant human IL-4 suitable for use in the invention is commercially available from multiple suppliers, including ProSpec-Tany TechnoGene Ltd., East Brunswick, NJ, USA (Cat. No. CYT-211) and ThermoFisher Scientific, Inc., Waltham, MA, USA (human IL-15 recombinant protein, Cat. No. Gibco CTP0043). The amino acid sequence of recombinant human IL-4 suitable for use in the invention is given in Table 2 (SEQ ID NO:5).

The term “IL-7” (also referred to herein as “IL7”) refers to a glycosylated tissue-derived cytokine known as interleukin 7, which may be obtained from stromal and epithelial cells, as well as from dendritic cells. Fry and Mackall, Blood 2002, 99, 3892-904. IL-7 can stimulate the development of T cells. IL-7 binds to the IL-7 receptor, a heterodimer consisting of IIL-7 receptor alpha and common gamma chain receptor, which in a series of signals important for T cell development within the thymus and survival within the periphery. Recombinant human IL-7 suitable for use in the invention is commercially available from multiple suppliers, including ProSpec-Tany TechnoGene Ltd., East Brunswick, NJ, USA (Cat. No. CYT-254) and ThermoFisher Scientific, Inc., Waltham, MA, USA (human IL-7 recombinant protein, Cat. No. Gibco PHC0071). The amino acid sequence of recombinant human IL-7 suitable for use in the invention is given in Table 2 (SEQ ID NO:6).

The term “IL-15” (also referred to herein as “IL15”) refers to the T cell growth factor known as interleukin-15, and includes all forms of IL-15 including human and mammalian forms, conservative amino acid substitutions, glycoforms, biosimilars, and variants thereof. IL-15 is described, e.g., in Fehniger and Caligiuri, Blood 2001, 97, 14-32, the disclosure of which is incorporated by reference herein. IL-15 shares β and γ signaling receptor subunits with IL-2. Recombinant human IL-15 is a single, non-glycosylated polypeptide chain containing 114 amino acids (and an N-terminal methionine) with a molecular mass of 12.8 kDa. Recombinant human IL-15 is commercially available from multiple suppliers, including ProSpec-Tany TechnoGene Ltd., East Brunswick, NJ, USA (Cat. No. CYT-230-b) and ThermoFisher Scientific, Inc., Waltham, MA, USA (human IL-15 recombinant protein, Cat. No. 34-8159-82). The amino acid sequence of recombinant human IL-15 suitable for use in the invention is given in Table 2 (SEQ ID NO:7).

The term “IL-21” (also referred to herein as “IL21”) refers to the pleiotropic cytokine protein known as interleukin-21, and includes all forms of IL-21 including human and mammalian forms, conservative amino acid substitutions, glycoforms, biosimilars, and variants thereof. IL-21 is described, e.g., in Spolski and Leonard, Nat. Rev. Drug. Disc. 2014, 13, 379-95, the disclosure of which is incorporated by reference herein. IL-21 is primarily produced by natural killer T cells and activated human CD4⁺ T cells. Recombinant human IL-21 is a single, non-glycosylated polypeptide chain containing 132 amino acids with a molecular mass of 15.4 kDa. Recombinant human IL-21 is commercially available from multiple suppliers, including ProSpec-Tany TechnoGene Ltd., East Brunswick, NJ, USA (Cat. No. CYT-408-b) and ThermoFisher Scientific, Inc., Waltham, MA, USA (human IL-21 recombinant protein, Cat. No. 14-8219-80). The amino acid sequence of recombinant human IL-21 suitable for use in the invention is given in Table 2 (SEQ ID NO:8).

The term “in vivo” refers to an event that takes place in a mammalian subject's body.

The term “ex vivo” refers to an event that takes place outside of a mammalian subject's body, in an artificial environment.

The term “in vitro” refers to an event that takes places in a test system. In vitro assays encompass cell-based assays in which alive or dead cells may be are employed and may also encompass a cell-free assay in which no intact cells are employed.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, or the manner of administration. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

The term “pharmaceutically acceptable salt” refers to salts derived from a variety of organic and inorganic counter ions known in the art. Pharmaceutically acceptable acid addition salts can be formed with inorganic acids and organic acids. Preferred inorganic acids from which salts can be derived include, for example, hydrochloric acid, hydrobromic acid, sulfuric acid, nitric acid and phosphoric acid. Preferred organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the invention is contemplated. Additional active pharmaceutical ingredients, such as other drugs, can also be incorporated into the described compositions, processes and methods.

The term “antigen” refers to a substance that induces an immune response. In some embodiments, an antigen is a molecule capable of being bound by an antibody or a T cell receptor (TCR) if presented by major histocompatibility complex (MHC) molecules. The term “antigen”, as used herein, also encompasses T cell epitopes. An antigen is additionally capable of being recognized by the immune system. In some embodiments, an antigen is capable of inducing a humoral immune response or a cellular immune response leading to the activation of B lymphocytes and/or T lymphocytes. In some cases, this may require that the antigen contains or is linked to a Th cell epitope. An antigen can also have one or more epitopes (e.g., B- and T-epitopes). In some embodiments, an antigen will preferably react, typically in a highly specific and selective manner, with its corresponding antibody or TCR and not with the multitude of other antibodies or TCRs which may be induced by their antigens.

The terms “antibody” and its plural form “antibodies” refer to whole immunoglobulins and any antigen-binding fragment (“antigen-binding portion”) or single chains thereof. An “antibody” further refers to a glycoprotein comprising at least two heavy (H) chains and two light (L) chains inter-connected by disulfide bonds, or an antigen-binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions of an antibody may be further subdivided into regions of hypervariability, which are referred to as complementarity determining regions (CDR) or hypervariable regions (HVR), and which can be interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen epitope or epitopes. The constant regions of the antibodies may mediate the binding of the immunoglobulin to host tissues or factors, including various cells of the immune system (e.g., effector cells) and the first component (Clq) of the classical complement system.

The terms “monoclonal antibody,” “mAb,” “monoclonal antibody composition,” or their plural forms refer to a preparation of antibody molecules of single molecular composition. A monoclonal antibody composition displays a single binding specificity and affinity for a particular epitope. Monoclonal antibodies specific to TNFRSF receptors can be made using knowledge and skill in the art of injecting test subjects with suitable antigen and then isolating hybridomas expressing antibodies having the desired sequence or functional characteristics. DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

The terms “antigen-binding portion” or “antigen-binding fragment” of an antibody (or simply “antibody portion” or “fragment”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and CH1 domains; (ii) a F(ab′)2 fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a domain antibody (dAb) fragment (Ward, et al., Nature, 1989, 341, 544-546), which may consist of a V_{H} or a V_{L} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv); see, e.g., Bird, et al., Science 1988, 242, 423-426; and Huston, et al., Proc. Natl. Acad. Sci. USA 1988, 85, 5879-5883). Such scFv antibodies are also intended to be encompassed within the terms “antigen-binding portion” or “antigen-binding fragment” of an antibody. These antibody fragments are obtained using conventional techniques known to those with skill in the art, and the fragments are screened for utility in the same manner as are intact antibodies.

The term “human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). The term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In an embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (such as a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, including a conjugate of the antibody and another active pharmaceutical ingredient or antibody. The terms “conjugate,” “antibody-drug conjugate”, “ADC,” or “immunoconjugate” refers to an antibody, or a fragment thereof, conjugated to another therapeutic moiety, which can be conjugated to antibodies described herein using methods available in the art.

The terms “humanized antibody,” “humanized antibodies,” and “humanized” are intended to refer to antibodies in which CDR sequences derived from the germline of another mammalian species, such as a mouse, have been grafted onto human framework sequences. Additional framework region modifications may be made within the human framework sequences. Humanized forms of non-human (for example, murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a 15 hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones, et al., Nature 1986, 321, 522-525; Riechmann, et al., Nature 1988, 332, 323-329; and Presta, Curr. Op. Struct. Biol. 1992, 2, 593-596. The TNFRSF agonists described herein may also be modified to employ any Fc variant which is known to impart an improvement (e.g., reduction) in effector function and/or FcR binding. The Fc variants may include, for example, any one of the amino acid substitutions disclosed in International Patent Application Publication Nos. WO 1988/07089 A1, WO 1996/14339 A1, WO 1998/05787 A1, WO 1998/23289 A1, WO 1999/51642 A1, WO 99/58572 A1, WO 2000/09560 A2, WO 2000/32767 A1, WO 2000/42072 A2, WO 2002/44215 A2, WO 2002/060919 A2, WO 2003/074569 A2, WO 2004/016750 A2, WO 2004/029207 A2, WO 2004/035752 A2, WO 2004/063351 A2, WO 2004/074455 A2, WO 2004/099249 A2, WO 2005/040217 A2, WO 2005/070963 A1, WO 2005/077981 A2, WO 2005/092925 A2, WO 2005/123780 A2, WO 2006/019447 A1, WO 2006/047350 A2, and WO 2006/085967 A2; and U.S. Pat. Nos. 5,648,260; 5,739,277; 5,834,250; 5,869,046; 6,096,871; 6,121,022; 6,194,551; 6,242,195; 6,277,375; 6,528,624; 6,538,124; 6,737,056; 6,821,505; 6,998,253; and 7,083,784; the disclosures of which are incorporated by reference herein.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

A “diabody” is a small antibody fragment with two antigen-binding sites. The fragments comprises a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., European Patent No. EP 404,097, International Patent Publication No. WO 93/11161; and Bolliger, et al., Proc. Natl. Acad. Sci. USA 1993, 90, 6444-6448.

The term “glycosylation” refers to a modified derivative of an antibody. An aglycoslated antibody lacks glycosylation. Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Aglycosylation may increase the affinity of the antibody for antigen, as described in U.S. Pat. Nos. 5,714,350 and 6,350,861. Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (alpha (1,6) fucosyltransferase), such that antibodies expressed in the Ms704, Ms705, and Ms709 cell lines lack fucose on their carbohydrates. The Ms704, Ms705, and Ms709 FUT8−/− cell lines were created by the targeted disruption of the FUT8 gene in CHO/DG44 cells using two replacement vectors (see e.g. U.S. Patent Publication No. 2004/0110704 or Yamane-Ohnuki, et al., Biotechnol. Bioeng., 2004, 87, 614-622). As another example, European Patent No. EP 1,176,195 describes a cell line with a functionally disrupted FUT8 gene, which encodes a fucosyl transferase, such that antibodies expressed in such a cell line exhibit hypofucosylation by reducing or eliminating the alpha 1,6 bond-related enzyme, and also describes cell lines which have a low enzyme activity for adding fucose to the N-acetylglucosamine that binds to the Fc region of the antibody or does not have the enzyme activity, for example the rat myeloma cell line YB2/0 (ATCC CRL 1662). International Patent Publication WO 03/035835 describes a variant CHO cell line, Lec 13 cells, with reduced ability to attach fucose to Asn(297)-linked carbohydrates, also resulting in hypofucosylation of antibodies expressed in that host cell (see also Shields, et al., J. Biol. Chem. 2002, 277, 26733-26740. International Patent Publication WO 99/54342 describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana, et al., Nat. Biotech. 1999, 17, 176-180). Alternatively, the fucose residues of the antibody may be cleaved off using a fucosidase enzyme. For example, the fucosidase alpha-L-fucosidase removes fucosyl residues from antibodies as described in Tarentino, et al., Biochem. 1975, 14, 5516-5523.

“Pegylation” refers to a modified antibody or fusion protein, or a fragment thereof, that typically is reacted with polyethylene glycol (PEG), such as a reactive ester or aldehyde derivative of PEG, under conditions in which one or more PEG groups become attached to the antibody or antibody fragment. Pegylation may, for example, increase the biological (e.g., serum) half life of the antibody. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C₁-C₁₀) alkoxy- or aryloxy-polyethylene glycol or polyethylene glycol-maleimide. The protein or antibody to be pegylated may be an aglycosylated protein or antibody. Methods for pegylation are known in the art and can be applied to the antibodies of the invention, as described for example in European Patent Nos. EP 0154316 and EP 0401384 and U.S. Pat. No. 5,824,778, the disclosures of each of which are incorporated by reference herein.

The terms “fusion protein” or “fusion polypeptide” refer to proteins that combine the properties of two or more individual proteins. Such proteins have at least two heterologous polypeptides covalently linked either directly or via an amino acid linker. The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order and may include more than one of either or both of the constituent polypeptides. The term encompasses conservatively modified variants, polymorphic variants, alleles, mutants, subsequences, interspecies homologs, and immunogenic fragments of the antigens that make up the fusion protein. Fusion proteins of the disclosure can also comprise additional copies of a component antigen or immunogenic fragment thereof. The fusion protein may contain one or more binding domains linked together and further linked to an Fc domain, such as an IgG Fc domain. Fusion proteins may be further linked together to mimic a monoclonal antibody and provide six or more binding domains. Fusion proteins may be produced by recombinant methods as is known in the art. Preparation of fusion proteins are known in the art and are described, e.g., in International Patent Application Publication Nos. WO 1995/027735 A1, WO 2005/103077 A1, WO 2008/025516 A1, WO 2009/007120 A1, WO 2010/003766 A1, WO 2010/010051 A1, WO 2010/078966 A1, U.S. Patent Application Publication Nos. US 2015/0125419 A1 and US 2016/0272695 A1, and U.S. Pat. No. 8,921,519, the disclosures of each of which are incorporated by reference herein.

The term “heterologous” when used with reference to portions of a nucleic acid or protein indicates that the nucleic acid or protein comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source, or coding regions from different sources. Similarly, a heterologous protein indicates that the protein comprises two or more subsequences that are not found in the same relationship to each other in nature (e.g., a fusion protein).

The term “conservative amino acid substitutions” means amino acid sequence modifications which do not abrogate the binding of an antibody or fusion protein to the antigen. Conservative amino acid substitutions include the substitution of an amino acid in one class by an amino acid of the same class, where a class is defined by common physicochemical amino acid side chain properties and high substitution frequencies in homologous proteins found in nature, as determined, for example, by a standard Dayhoff frequency exchange matrix or BLOSUM matrix. Six general classes of amino acid side chains have been categorized and include: Class I (Cys); Class II (Ser, Thr, Pro, Ala, Gly); Class III (Asn, Asp, Gln, Glu); Class IV (His, Arg, Lys); Class V (Ile, Leu, Val, Met); and Class VI (Phe, Tyr, Trp). For example, substitution of an Asp for another class III residue such as Asn, Gln, or Glu, is a conservative substitution. Thus, a predicted nonessential amino acid residue in an antibody is preferably replaced with another amino acid residue from the same class. Methods of identifying amino acid conservative substitutions which do not eliminate antigen binding are well-known in the art (see, e.g., Brummell, et al., Biochemistry 1993, 32, 1180-1187; Kobayashi, et al., Protein Eng. 1999, 12, 879-884 (1999); and Burks, et al., Proc. Natl. Acad. Sci. USA 1997, 94, 412-417.

The terms “sequence identity,” “percent identity,” and “sequence percent identity” (or synonyms thereof, e.g., “99% identical”) in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. Suitable programs to determine percent sequence identity include for example the BLAST suite of programs available from the U.S. Government's National Center for Biotechnology Information BLAST web site. Comparisons between two sequences can be carried using either the BLASTN or BLASTP algorithm. BLASTN is used to compare nucleic acid sequences, while BLASTP is used to compare amino acid sequences. ALIGN, ALIGN-2 (Genentech, South San Francisco, California) or MegAlign, available from DNASTAR, are additional publicly available software programs that can be used to align sequences. One skilled in the art can determine appropriate parameters for maximal alignment by particular alignment software. In certain embodiments, the default parameters of the alignment software are used.

Certain embodiments of the present invention comprise a variant of an antibody or fusion protein. As used herein, the term “variant” encompasses but is not limited to antibodies or fusion proteins which comprise an amino acid sequence which differs from the amino acid sequence of a reference antibody by way of one or more substitutions, deletions and/or additions at certain positions within or adjacent to the amino acid sequence of the reference antibody. The variant may comprise one or more conservative substitutions in its amino acid sequence as compared to the amino acid sequence of a reference antibody. Conservative substitutions may involve, e.g., the substitution of similarly charged or uncharged amino acids. The variant retains the ability to specifically bind to the antigen of the reference antibody.

Nucleic acid sequences implicitly encompass conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues. Batzer, et al., Nucleic Acid Res. 1991, 19, 5081; Ohtsuka, et al., J. Biol. Chem. 1985, 260, 2605-2608; Rossolini, et al., Mol. Cell. Probes 1994, 8, 91-98. The term nucleic acid is used interchangeably with cDNA, mRNA, oligonucleotide, and polynucleotide.

The term “biosimilar” means a biological product, including a monoclonal antibody or fusion protein, that is highly similar to a U.S. licensed reference biological product notwithstanding minor differences in clinically inactive components, and for which there are no clinically meaningful differences between the biological product and the reference product in terms of the safety, purity, and potency of the product. Furthermore, a similar biological or “biosimilar” medicine is a biological medicine that is similar to another biological medicine that has already been authorized for use by the European Medicines Agency. The term “biosimilar” is also used synonymously by other national and regional regulatory agencies. Biological products or biological medicines are medicines that are made by or derived from a biological source, such as a bacterium or yeast. They can consist of relatively small molecules such as human insulin or erythropoietin, or complex molecules such as monoclonal antibodies. For example, if the reference monoclonal antibody is rituximab, an biosimilar monoclonal antibody approved by drug regulatory authorities with reference to rituximab is a “biosimilar to” rituximab or is a “biosimilar thereof” of rituximab. In Europe, a similar biological or “biosimilar” medicine is a biological medicine that is similar to another biological medicine that has already been authorized for use by the European Medicines Agency (EMA). The relevant legal basis for similar biological applications in Europe is Article 6 of Regulation (EC) No 726/2004 and Article 10(4) of Directive 2001/83/EC, as amended and therefore in Europe, the biosimilar may be authorised, approved for authorisation or subject of an application for authorisation under Article 6 of Regulation (EC) No 726/2004 and Article 10(4) of Directive 2001/83/EC. The already authorized original biological medicinal product may be referred to as a “reference medicinal product” in Europe. Some of the requirements for a product to be considered a biosimilar are outlined in the CHMP Guideline on Similar Biological Medicinal Products. In addition, product specific guidelines, including guidelines relating to monoclonal antibody biosimilars, are provided on a product-by-product basis by the EMA and published on its website. A biosimilar as described herein may be similar to the reference medicinal product by way of quality characteristics, biological activity, mechanism of action, safety profiles and/or efficacy. In addition, the biosimilar may be used or be intended for use to treat the same conditions as the reference medicinal product. Thus, a biosimilar as described herein may be deemed to have similar or highly similar quality characteristics to a reference medicinal product. Alternatively, or in addition, a biosimilar as described herein may be deemed to have similar or highly similar biological activity to a reference medicinal product. Alternatively, or in addition, a biosimilar as described herein may be deemed to have a similar or highly similar safety profile to a reference medicinal product. Alternatively, or in addition, a biosimilar as described herein may be deemed to have similar or highly similar efficacy to a reference medicinal product. As described herein, a biosimilar in Europe is compared to a reference medicinal product which has been authorised by the EMA. However, in some instances, the biosimilar may be compared to a biological medicinal product which has been authorised outside the European Economic Area (a non-EEA authorised “comparator”) in certain studies. Such studies include for example certain clinical and in vivo non-clinical studies. As used herein, the term “biosimilar” also relates to a biological medicinal product which has been or may be compared to a non-EEA authorised comparator. Certain biosimilars are proteins such as antibodies, antibody fragments (for example, antigen binding portions) and fusion proteins. A protein biosimilar may have an amino acid sequence that has minor modifications in the amino acid structure (including for example deletions, additions, and/or substitutions of amino acids) which do not significantly affect the function of the polypeptide. The biosimilar may comprise an amino acid sequence having a sequence identity of 97% or greater to the amino acid sequence of its reference medicinal product, e.g., 97%, 98%, 99% or 100%. The biosimilar may comprise one or more post-translational modifications, for example, although not limited to, glycosylation, oxidation, deamidation, and/or truncation which is/are different to the post-translational modifications of the reference medicinal product, provided that the differences do not result in a change in safety and/or efficacy of the medicinal product. The biosimilar may have an identical or different glycosylation pattern to the reference medicinal product. Particularly, although not exclusively, the biosimilar may have a different glycosylation pattern if the differences address or are intended to address safety concerns associated with the reference medicinal product. Additionally, the biosimilar may deviate from the reference medicinal product in for example its strength, pharmaceutical form, formulation, excipients and/or presentation, providing safety and efficacy of the medicinal product is not compromised. In some embodiments, a biosimilar is provided as a composition which further comprises one or more excipients, wherein the one or more excipients are the same or different to the excipients comprised in a reference medicinal product or reference biological product. The biosimilar may comprise differences in for example pharmacokinetic (PK) and/or pharmacodynamic (PD) profiles as compared to the reference medicinal product but is still deemed sufficiently similar to the reference medicinal product as to be authorised or considered suitable for authorisation. In certain circumstances, the biosimilar exhibits different binding characteristics as compared to the reference medicinal product, wherein the different binding characteristics are considered by a Regulatory Authority such as the EMA not to be a barrier for authorisation as a similar biological product. The term “biosimilar” is also used synonymously by other national and regional regulatory agencies.

As used herein, the term “4-1BB agonist” may refer to any antibody or protein that specifically binds to 4-1BB (CD137) antigen. By “specifically binds” it is meant that the binding molecules exhibit essentially background binding to non-4-1BB molecules. The 4-1BB agonist may be any 4-1BB agonist known in the art. In particular, it is one of the 4-1BB agonists described in more detail herein. An isolated binding molecule that specifically binds 4-1BB may, however, have cross-reactivity to 4-1BB molecules from other species. 4-1BB agonistic antibodies and proteins may also specifically bind to e.g., human 4-1BB (h4-1BB or hCD137) on T cells.

As used herein, the term “OX40 agonist” may refer to any antibody or protein that specifically binds to OX40 (CD134) antigen. By “specifically binds” it is meant that the binding molecules exhibit essentially background binding to non-OX40 molecules. The OX40 agonist may be any OX40 agonist known in the art. In particular, it is one of the OX40 agonists described in more detail herein. An isolated binding molecule that specifically binds OX40 may, however, have cross-reactivity to OX40 molecules from other species. OX40 agonistic antibodies and proteins may also specifically bind to e.g., human OX40 (hOX40 or hCD134) on T cells.

As used herein, the term “CD27 agonist” may refer to any antibody or protein that specifically binds to CD27 antigen. By “specifically binds” it is meant that the binding molecules exhibit essentially background binding to non-CD27 molecules. The CD27 agonist may be any CD27 agonist known in the art. In particular, it is one of the CD27 agonists described in more detail herein. An isolated binding molecule that specifically binds CD27 may, however, have cross-reactivity to CD27 molecules from other species. CD27 agonistic antibodies and proteins may also specifically bind to e.g., human CD27 (hCD27) on T cells.

As used herein, the term “GITR agonist” includes molecules that contain at least one antigen binding site that specifically binds to GITR (CD357). By “specifically binds” it is meant that the binding molecules exhibit essentially background binding to non-GITR molecules. The GITR agonist may be any GITR agonist known in the art. In particular, it is one of the GITR agonists described in more detail herein. An isolated binding molecule that specifically binds GITR may, however, have cross-reactivity to GITR molecules from other species. GITR agonistic antibodies and proteins may also specifically bind to e.g., human GITR (hGITR) on T cells and dendritic cells.

As used herein, the term “HVEM agonist” includes molecules that contain at least one antigen binding site that specifically binds to HVEM (CD270). By “specifically binds” it is meant that the binding molecules exhibit essentially background binding to non-HVEM molecules. The HVEM agonist may be any HVEM agonist known in the art. In particular, it is one of the HVEM agonists described in more detail herein. An isolated binding molecule that specifically binds HVEM may, however, have cross-reactivity to HVEM molecules from other species. HVEM agonistic antibodies and proteins may also specifically bind to e.g., human HVEM (hHVEM) on T cells.

The term “hematological malignancy” refers to mammalian cancers and tumors of the hematopoietic and lymphoid tissues, including but not limited to tissues of the blood, bone marrow, lymph nodes, and lymphatic system. Hematological malignancies are also referred to as “liquid tumors.” Hematological malignancies include, but are not limited to, acute lymphoblastic leukemia (ALL), chronic lymphocytic lymphoma (CLL), small lymphocytic lymphoma (SLL), acute myelogenous leukemia (AML), chronic myelogenous leukemia (CML), acute monocytic leukemia (AMoL), Hodgkin's lymphoma, and non-Hodgkin's lymphomas. The term “B cell hematological malignancy” refers to hematological malignancies that affect B cells.

The term “solid tumor” refers to an abnormal mass of tissue that usually does not contain cysts or liquid areas. Solid tumors may be benign or malignant. The term “solid tumor cancer” refers to malignant, neoplastic, or cancerous solid tumors. Solid tumor cancers include, but are not limited to, sarcomas, carcinomas, and lymphomas, such as cancers of the lung, breast, prostate, colon, rectum, and bladder. The tissue structure of solid tumors includes interdependent tissue compartments including the parenchyma (cancer cells) and the supporting stromal cells in which the cancer cells are dispersed and which may provide a supporting microenvironment.

The term “microenvironment,” as used herein, may refer to the solid or hematological tumor microenvironment as a whole or to an individual subset of cells within the microenvironment. The tumor microenvironment, as used herein, refers to a complex mixture of “cells, soluble factors, signaling molecules, extracellular matrices, and mechanical cues that promote neoplastic transformation, support tumor growth and invasion, protect the tumor from host immunity, foster therapeutic resistance, and provide niches for dominant metastases to thrive,” as described in Swartz, et al., Cancer Res., 2012, 72, 2473. Although tumors express antigens that should be recognized by T cells, tumor clearance by the immune system is rare because of immune suppression by the microenvironment.

For the avoidance of doubt, it is intended herein that particular features (for example integers, characteristics, values, uses, diseases, formulae, compounds or groups) described in conjunction with a particular aspect, embodiment or example of the invention are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any disclosed embodiments. The invention extends to any novel one, or novel combination, of the features disclosed in this specification (including any accompanying claims, abstract and drawings), or to any novel one, or any novel combination, of the steps of any method or process so disclosed.

The terms “about” and “approximately” mean within a statistically meaningful range of a value. Such a range can be within an order of magnitude, preferably within 50%, more preferably within 20%, more preferably still within 10%, and even more preferably within 5% of a given value or range. The allowable variation encompassed by the terms “about” or “approximately” depends on the particular system under study, and can be readily appreciated by one of ordinary skill in the art. Moreover, as used herein, the terms “about” and “approximately” mean that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

The transitional terms “comprising,” “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All compositions, methods, and kits described herein that embody the present invention can, in alternate embodiments, be more specifically defined by any of the transitional terms “comprising,” “consisting essentially of,” and “consisting of.”

### 4-1BB (CD137) Agonists

In an embodiment, the TNFRSF agonist is a 4-1BB (CD137) agonist. The 4-1BB agonist may be any 4-1BB binding molecule known in the art. The 4-1BB binding molecule may be a monoclonal antibody or fusion protein capable of binding to human or mammalian 4-1BB. The 4-1BB agonists or 4-1BB binding molecules may comprise an immunoglobulin heavy chain of any isotype (e.g., IgG, IgE, IgM, IgD, IgA, and IgY), class (e.g., IgG1, IgG2, IgG3, IgG4, IgA1 and IgA2) or subclass of immunoglobulin molecule. The 4-1BB agonist or 4-1BB binding molecule may have both a heavy and a light chain. As used herein, the term binding molecule also includes antibodies (including full length antibodies), monoclonal antibodies (including full length monoclonal antibodies), polyclonal antibodies, multispecific antibodies (e.g., bispecific antibodies), human, humanized or chimeric antibodies, and antibody fragments, e.g., Fab fragments, F(ab′) fragments, fragments produced by a Fab expression library, epitope-binding fragments of any of the above, and engineered forms of antibodies, e.g., scFv molecules, that bind to 4-1BB. In an embodiment, the 4-1BB agonist is an antigen binding protein that is a fully human antibody. In an embodiment, the 4-1BB agonist is an antigen binding protein that is a humanized antibody. In some embodiments, 4-1BB agonists for use in the presently disclosed methods and compositions include anti-4-1BB antibodies, human anti-4-1BB antibodies, mouse anti-4-1BB antibodies, mammalian anti-4-1BB antibodies, monoclonal anti-4-1BB antibodies, polyclonal anti-4-1BB antibodies, chimeric anti-4-1BB antibodies, anti-4-1BB adnectins, anti-4-1BB domain antibodies, single chain anti-4-1BB fragments, heavy chain anti-4-1BB fragments, light chain anti-4-1BB fragments, anti-4-1BB fusion proteins, and fragments, derivatives, conjugates, variants, or biosimilars thereof. Agonistic anti-4-1BB antibodies are known to induce strong immune responses. Lee, et al., PLOS One 2013, 8, e69677. In a preferred embodiment, the 4-1BB agonist is an agonistic, anti-4-1BB humanized or fully human monoclonal antibody (i.e., an antibody derived from a single cell line). In an embodiment, the 4-1BB agonist is EU-101 (Eutilex Co. Ltd.), utomilumab, or urelumab, or a fragment, derivative, conjugate, variant, or biosimilar thereof. In a preferred embodiment, the 4-1BB agonist is utomilumab or urelumab, or a fragment, derivative, conjugate, variant, or biosimilar thereof.

In a preferred embodiment, the 4-1BB agonist or 4-1BB binding molecule may also be a fusion protein. In a preferred embodiment, a multimeric 4-1BB agonist, such as a trimeric or hexameric 4-1BB agonist (with three or six ligand binding domains), may induce superior receptor (4-1BBL) clustering and internal cellular signaling complex formation compared to an agonistic monoclonal antibody, which typically possesses two ligand binding domains. Trimeric (trivalent) or hexameric (or hexavalent) or greater fusion proteins comprising three TNFRSF binding domains and IgG1-Fc and optionally further linking two or more of these fusion proteins are described, e.g., in Gieffers, et al., Mol. Cancer Therapeutics 2013, 12, 2735-47.

Agonistic 4-1BB antibodies and fusion proteins are known to induce strong immune responses. In a preferred embodiment, the 4-1BB agonist is a monoclonal antibody or fusion protein that binds specifically to 4-1BB antigen in a manner sufficient to reduce toxicity. In some embodiments, the 4-1BB agonist is an agonistic 4-1BB monoclonal antibody or fusion protein that abrogates antibody-dependent cellular toxicity (ADCC), for example NK cell cytotoxicity. In some embodiments, the 4-1BB agonist is an agonistic 4-1BB monoclonal antibody or fusion protein that abrogates antibody-dependent cell phagocytosis (ADCP). In some embodiments, the 4-1BB agonist is an agonistic 4-1BB monoclonal antibody or fusion protein that abrogates complement-dependent cytotoxicity (CDC). In some embodiments, the 4-1BB agonist is an agonistic 4-1BB monoclonal antibody or fusion protein which abrogates Fc region functionality.

In some embodiments, the 4-1BB agonists are characterized by binding to human 4-1BB (SEQ ID NO:9) with high affinity and agonistic activity. In an embodiment, the 4-1BB agonist is a binding molecule that binds to human 4-1BB (SEQ ID NO:9). In an embodiment, the 4-1BB agonist is a binding molecule that binds to murine 4-1BB (SEQ ID NO:10). The amino acid sequences of 4-1BB antigen to which a 4-1BB agonist or binding molecule binds are summarized in Table 3.

TABLE 3
Amino acid sequences of 4-1BB antigens.

| Identifier | Sequence (One-Letter Amino Acid Symbols) |
|---|---|
| SEQ ID NO: 9 | MGNSCYNIVA TLLLVLNFER TRSLQDPCSN CPAGTFCDNN RNQICSPCPP NSFSSAGGQR 60 |
| human 4-1BB, | TCDICRQCKG VFRTRKECSS TSNAECDCTP GFHCLGAGCS MCEQDCKQGQ ELTKKGCKDC 120 |
| Tumor necrosis | CFGTFNDQKR GICRPWTNCS LDGKSVLVNG TKERDVVCGP SPADLSPGAS SVTPPAPARE 180 |
| factor receptor | PGHSPQIISF FLALTSTALL FLLFFLTLRF SVVKRGRKKL LYIFKQPFMR PVQTTQEEDG 240 |
| superfamily, | CSCRFPEEEE GGCEL 255 |
| member 9 (Homo | |
| sapiens) | |
| SEQ ID NO: 10 | MGNNCYNVVV IVLLLVGCEK VGAVQNSCDN CQPGTFCRKY NPVCKSCPPS TFSSIGGQPN 60 |
| murine 4-1BB, | CNICRVCAGY FRFKKFCSST HNAECECIEG FHCLGPQCTR CEKDCRPGQE LTKQGCKTCS 120 |
| Tumor necrosis | LGTFNDQNGT GVCRPWTNCS LDGRSVLKTG TTEKDVVCGP PVVSFSPSTT ISVTPEGGPG 160 |
| factor receptor | GHSLQVLTLF LALTSALLLA LIFITLLFSV LKWIRKKFPH IFKQPFKKTT GAAQEEDACS 240 |
| superfamily, | CRCPQEEEGG GGGYEL 256 |
| member 9 (Mus | |
| musculus) | |

In some embodiments, the compositions, processes and methods described include a 4-1BB agonist that binds human or murine 4-1BB with a K_{D} of about 100 pM or lower, binds human or murine 4-1BB with a K_{D} of about 90 pM or lower, binds human or murine 4-1BB with a K_{D} of about 80 pM or lower, binds human or murine 4-1BB with a K_{D} of about 70 pM or lower, binds human or murine 4-1BB with a K_{D} of about 60 pM or lower, binds human or murine 4-1BB with a K_{D} of about 50 pM or lower, binds human or murine 4-1BB with a K_{D} of about 40 pM or lower, or binds human or murine 4-1BB with a K_{D} of about 30 pM or lower.

In some embodiments, the compositions, processes and methods described include a 4-1BB agonist that binds to human or murine 4-1BB with a k_{assoc} of about 7.5×10⁵ l/M·s or faster, binds to human or murine 4-1BB with a k_{assoc} of about 7.5×10⁵ l/M·s or faster, binds to human or murine 4-1BB with a k_{assoc} of about 8×10⁵ l/M·s or faster, binds to human or murine 4-1BB with a k_{assoc} of about 8.5×10⁵ l/M·s or faster, binds to human or murine 4-1BB with a k_{assoc} of about 9×10⁵ l/M·s or faster, binds to human or murine 4-1BB with a k_{assoc} of about 9.5×10⁵ l/M·s or faster, or binds to human or murine 4-1BB with a k_{assoc} of about 1×10⁶ l/M·s or faster.

In some embodiments, the compositions, processes and methods described include a 4-1BB agonist that binds to human or murine 4-1BB with a k_{dissoc} of about 2×10⁻⁵ l/s or slower, binds to human or murine 4-1BB with a k_{dissoc} of about 2.1×10⁻⁵ l/s or slower, binds to human or murine 4-1BB with a k_{dissoc} of about 2.2×10⁻⁵ l/s or slower, binds to human or murine 4-1BB with a k_{dissoc} of about 2.3×10⁻⁵ l/s or slower, binds to human or murine 4-1BB with a k_{dissoc} of about 2.4×10⁻⁵ l/s or slower, binds to human or murine 4-1BB with a k_{dissoc} of about 2.5×10⁻⁵ l/s or slower, binds to human or murine 4-1BB with a k_{dissoc} of about 2.6×10⁻⁵ l/s or slower or binds to human or murine 4-1BB with a k_{dissoc} of about 2.7×10⁻⁵ l/s or slower, binds to human or murine 4-1BB with a k_{dissoc} of about 2.8×10⁻⁵ l/s or slower, binds to human or murine 4-1BB with a k_{dissoc} of about 2.9×10⁻⁵ l/s or slower, or binds to human or murine 4-1BB with a k_{dissoc} of about 3×10⁻⁵ l/s or slower.

In some embodiments, the compositions, processes and methods described include a 4-1BB agonist that binds to human or murine 4-1BB with an IC₅₀ of about 10 nM or lower, binds to human or murine 4-1BB with an IC₅₀ of about 9 nM or lower, binds to human or murine 4-1BB with an IC₅₀ of about 8 nM or lower, binds to human or murine 4-1BB with an IC₅₀ of about 7 nM or lower, binds to human or murine 4-1BB with an IC₅₀ of about 6 nM or lower, binds to human or murine 4-1BB with an IC₅₀ of about 5 nM or lower, binds to human or murine 4-1BB with an IC₅₀ of about 4 nM or lower, binds to human or murine 4-1BB with an IC₅₀ of about 3 nM or lower, binds to human or murine 4-1BB with an IC₅₀ of about 2 nM or lower, or binds to human or murine 4-1BB with an IC₅₀ of about 1 nM or lower.

In a preferred embodiment, the 4-1BB agonist is utomilumab, also known as PF-05082566 or MOR-7480, or a fragment, derivative, variant, or biosimilar thereof. Utomilumab is available from Pfizer, Inc. Utomilumab is an immunoglobulin G2-lambda, anti-[Homo sapiens TNFRSF9 (tumor necrosis factor receptor (TNFR) superfamily member 9, 4-1BB, T cell antigen ILA, CD137)], Homo sapiens (fully human) monoclonal antibody. The amino acid sequences of utomilumab are set forth in Table 4. Utomilumab comprises glycosylation sites at Asn59 and Asn292; heavy chain intrachain disulfide bridges at positions 22-96 (V_{H}-V_{L}), 143-199 (C_{H}1-C_{L}), 256-316 (C_{H}2) and 362-420 (C_{H}3); light chain intrachain disulfide bridges at positions 22′-87′ (V_{H}-V_{L}) and 136′-195′ (C_{H}1-C_{L}); interchain heavy chain-heavy chain disulfide bridges at IgG2A isoform positions 218-218, 219-219, 222-222, and 225-225, at IgG2A/B isoform positions 218-130, 219-219, 222-222, and 225-225, and at IgG2B isoform positions 219-130 (2), 222-222, and 225-225; and interchain heavy chain-light chain disulfide bridges at IgG2A isoform positions 130-213′ (2), IgG2A/B isoform positions 218-213′ and 130-213′, and at IgG2B isoform positions 218-213′ (2). The preparation and properties of utomilumab and its variants and fragments are described in U.S. Pat. Nos. 8,821,867; 8,337,850; and 9,468,678, and International Patent Application Publication No. WO 2012/032433 A1, the disclosures of each of which are incorporated by reference herein. Preclinical characteristics of utomilumab are described in Fisher, et al., Cancer Immunolog. & Immunother. 2012, 61, 1721-33. Current clinical trials of utomilumab in a variety of hematological and solid tumor indications include U.S. National Institutes of Health clinicaltrials.gov identifiers NCT02444793, NCT01307267, NCT02315066, and NCT02554812.

In an embodiment, a 4-1BB agonist comprises a heavy chain given by SEQ ID NO:11 and a light chain given by SEQ ID NO:12. In an embodiment, a 4-1BB agonist comprises heavy and light chains having the sequences shown in SEQ ID NO:11 and SEQ ID NO:12, respectively, or antigen binding fragments, Fab fragments, single-chain variable fragments (scFv), variants, or conjugates thereof. In an embodiment, a 4-1BB agonist comprises heavy and light chains that are each at least 99% identical to the sequences shown in SEQ ID NO:11 and SEQ ID NO:12, respectively. In an embodiment, a 4-1BB agonist comprises heavy and light chains that are each at least 98% identical to the sequences shown in SEQ ID NO:11 and SEQ ID NO:12, respectively. In an embodiment, a 4-1BB agonist comprises heavy and light chains that are each at least 97% identical to the sequences shown in SEQ ID NO:11 and SEQ ID NO:12, respectively. In an embodiment, a 4-1BB agonist comprises heavy and light chains that are each at least 96% identical to the sequences shown in SEQ ID NO:11 and SEQ ID NO:12, respectively. In an embodiment, a 4-1BB agonist comprises heavy and light chains that are each at least 95% identical to the sequences shown in SEQ ID NO:11 and SEQ ID NO:12, respectively.

In an embodiment, the 4-1BB agonist comprises the heavy and light chain CDRs or variable regions (VRs) of utomilumab. In an embodiment, the 4-1BB agonist heavy chain variable region (V_{H}) comprises the sequence shown in SEQ ID NO:13, and the 4-1BB agonist light chain variable region (V_{L}) comprises the sequence shown in SEQ ID NO:14, and conservative amino acid substitutions thereof. In an embodiment, a 4-1BB agonist comprises V_{H} and V_{L} regions that are each at least 99% identical to the sequences shown in SEQ ID NO:13 and SEQ ID NO:14, respectively. In an embodiment, a 4-1BB agonist comprises V_{H} and V_{L} regions that are each at least 98% identical to the sequences shown in SEQ ID NO:13 and SEQ ID NO:14, respectively. In an embodiment, a 4-1BB agonist comprises V_{H} and V_{L} regions that are each at least 97% identical to the sequences shown in SEQ ID NO:13 and SEQ ID NO:14, respectively. In an embodiment, a 4-1BB agonist comprises V_{H} and V_{L} regions that are each at least 96% identical to the sequences shown in SEQ ID NO: 13 and SEQ ID NO:14, respectively. In an embodiment, a 4-1BB agonist comprises V_{H} and V_{L} regions that are each at least 95% identical to the sequences shown in SEQ ID NO:13 and SEQ ID NO:14, respectively. In an embodiment, a 4-1BB agonist comprises an scFv antibody comprising V_{H} and V_{L} regions that are each at least 99% identical to the sequences shown in SEQ ID NO:13 and SEQ ID NO:14.

In an embodiment, a 4-1BB agonist comprises heavy chain CDR1, CDR2 and CDR3 domains having the sequences set forth in SEQ ID NO:15, SEQ ID NO:16, and SEQ ID NO:17, respectively, and conservative amino acid substitutions thereof, and light chain CDR1, CDR2 and CDR3 domains having the sequences set forth in SEQ ID NO:18, SEQ ID NO:19, and SEQ ID NO:20, respectively, and conservative amino acid substitutions thereof.

In an embodiment, the 4-1BB agonist is a 4-1BB agonist biosimilar monoclonal antibody approved by drug regulatory authorities with reference to utomilumab. In an embodiment, the biosimilar monoclonal antibody comprises an 4-1BB antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is utomilumab. In some embodiments, the one or more post-translational modifications are selected from one or more of: glycosylation, oxidation, deamidation, and truncation. In some embodiments, the biosimilar is a 4-1BB agonist antibody authorized or submitted for authorization, wherein the 4-1BB agonist antibody is provided in a formulation which differs from the formulations of a reference medicinal product or reference biological product, wherein the reference medicinal product or reference biological product is utomilumab. The 4-1BB agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is utomilumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is utomilumab.

TABLE 4
Amino acid sequences for 4-1BB agonist antibodies related to utomilumab.

| Identifier | Sequence (One-Letter Amino Acid Symbols) |
|---|---|
| SEQ ID NO: 11 | EVQLVQSGAE VKKPGESLRI SCKGSGYSFS TYWISWVRQM PGKGLEWMGK IYPGDSYTNY 60 |
| heavy chain for | SPSFQGQVTI SADKSISTAY LQWSSLKASD TAMYYCARGY GIFDYWGQGT LVTVSSASTK 120 |
| utomilumab | GPSVFPLAPC SRSTSESTAA LGCLVKDYFP EPVTVSWNSG ALTSGVHTFP AVLQSSGLYS 160 |
| | LSSVVTVPSS NFGTQTYTCN VDHKPSNTKV DKTVERKCCV ECPPCPAPPV AGPSVFLFPP 240 |
| | KPKDTLMISR TPEVTCVVVD VSHEDPEVQF NWYVDGVEVH NAKTKPREEQ FNSTFRVVSV 300 |
| | LTVVEQDWLN GKEYKCKVSN KGLPAPIEKT ISKTKGQPRE PQVYTLPPSR EEMTKNQVSL 360 |
| | TCLVKGFYPS DIAVEWESNG QPENNYKTTP PMLDSDGSFF LYSKLTVDKS RWQQGNVFSC 420 |
| | SVMHEALHNH YTQKSLSLSP G 441 |
| SEQ ID NO: 12 | SYELTQPPSV SVSPGQTASI TCSGDNIGDQ YAHWYQQKPG QSPVLVIYQD KNRPSGIPER 60 |
| light chain for | FSGSNSGNTA TLTISGTQAM DEADYYCATY TGFGSLAVFG GGTKLTVLGQ PKAAPSVTLF 120 |
| utomilumab | PPSSEELQAN KATLVCLISD FYPGAVTVAW KADSSPVKAG VETTTPSKQS NNKYAASSYL 180 |
| | SLTPEQWKSH RSYSCQVTHE GSTVEKTVAP TECS 214 |
| SEQ ID NO: 13 | EVQLVQSGAE VKKPGESLRI SCKGSGYSFS TYWISWVRQM PGKGLEWMG KIYPGDSYTN 60 |
| heavy chain | YSPSFQGQVT ISADKSISTA YLQWSSLKAS DTAMYYCARG YGIFDYWGQ GTLVTVSS 118 |
| variable region | |
| for utomilumab | |
| SEQ ID NO: 14 | SYELTQPPSV SVSPGQTASI TCSGDNIGDQ YAHWYQQKPG QSPVLVIYQD KNRPSGIPER 60 |
| light chain | FSGSNSGNTA TLTISGTQAM DEADYYCATY TGFGSLAVFG GGTKLTVL 108 |
| variable region | |
| for utomilumab | |
| SEQ ID NO: 15 | STYWIS 6 |
| heavy chain CDR1 | |
| for utomilumab | |
| SEQ ID NO: 16 | KIYPGDSYTN YSPSFQG 17 |
| heavy chain CDR2 | |
| for utomilumab | |
| SEQ ID NO: 17 | RGYGIFDY 8 |
| heavy chain CDR3 | |
| for utomilumab | |
| SEQ ID NO: 18 | SGDNIGDQYA H 11 |
| light chain CDR1 | |
| for utomilumab | |
| SEQ ID NO: 19 | QDKNRPS 7 |
| light chain CDR2 | |
| for utomilumab | |
| SEQ ID NO: 20 | ATYTGFGSLA V 11 |
| light chain CDR3 | |
| for utomilumab | |

In a preferred embodiment, the 4-1BB agonist is the monoclonal antibody urelumab, also known as BMS-663513 and 20H4.9.h4a, or a fragment, derivative, variant, or biosimilar thereof. Urelumab is available from Bristol-Myers Squibb, Inc., and Creative Biolabs, Inc. Urelumab is an immunoglobulin G4-kappa, anti-[Homo sapiens TNFRSF9 (tumor necrosis factor receptor superfamily member 9, 4-1BB, T cell antigen ILA, CD137)], Homo sapiens (fully human) monoclonal antibody. The amino acid sequences of urelumab are set forth in Table 5. Urelumab comprises N-glycosylation sites at positions 298 (and 298″); heavy chain intrachain disulfide bridges at positions 22-95 (V_{H}-V_{L}), 148-204 (C_{H}1-C_{L}), 262-322 (C_{H}2) and 368-426 (C_{H}3) (and at positions 22″-95″, 148″-204″, 262″-322″, and 368″-426″); light chain intrachain disulfide bridges at positions 23′-88′ (V_{H}-V_{L}) and 136′-196′ (C_{H}1-C_{L}) (and at positions 23′″-88′″ and 136′″-196′″); interchain heavy chain-heavy chain disulfide bridges at positions 227-227″ and 230-230″; and interchain heavy chain-light chain disulfide bridges at 135-216′ and 135″-216′″. The preparation and properties of urelumab and its variants and fragments are described in U.S. Pat. Nos. 7,288,638 and 8,962,804, the disclosures of which are incorporated by reference herein. The preclinical and clinical characteristics of urelumab are described in Segal, et al., Clin. Cancer Res. 2016, available at http:/dx.doi.org/10.1158/1078-0432.CCR-16-1272. Current clinical trials of urelumab in a variety of hematological and solid tumor indications include U.S. National Institutes of Health clinicaltrials.gov identifiers NCT01775631, NCT02110082, NCT02253992, and NCT01471210.

In an embodiment, a 4-1BB agonist comprises a heavy chain given by SEQ ID NO:21 and a light chain given by SEQ ID NO:22. In an embodiment, a 4-1BB agonist comprises heavy and light chains having the sequences shown in SEQ ID NO:21 and SEQ ID NO:22, respectively, or antigen binding fragments, Fab fragments, single-chain variable fragments (scFv), variants, or conjugates thereof. In an embodiment, a 4-1BB agonist comprises heavy and light chains that are each at least 99% identical to the sequences shown in SEQ ID NO:21 and SEQ ID NO:22, respectively. In an embodiment, a 4-1BB agonist comprises heavy and light chains that are each at least 98% identical to the sequences shown in SEQ ID NO:21 and SEQ ID NO:22, respectively. In an embodiment, a 4-1BB agonist comprises heavy and light chains that are each at least 97% identical to the sequences shown in SEQ ID NO:21 and SEQ ID NO:22, respectively. In an embodiment, a 4-1BB agonist comprises heavy and light chains that are each at least 96% identical to the sequences shown in SEQ ID NO:21 and SEQ ID NO:22, respectively. In an embodiment, a 4-1BB agonist comprises heavy and light chains that are each at least 95% identical to the sequences shown in SEQ ID NO:21 and SEQ ID NO:22, respectively.

In an embodiment, the 4-1BB agonist comprises the heavy and light chain CDRs or variable regions (VRs) of urelumab. In an embodiment, the 4-1BB agonist heavy chain variable region (V_{H}) comprises the sequence shown in SEQ ID NO:23, and the 4-1BB agonist light chain variable region (V_{L}) comprises the sequence shown in SEQ ID NO:24, and conservative amino acid substitutions thereof. In an embodiment, a 4-1BB agonist comprises V_{H} and V_{L} regions that are each at least 99% identical to the sequences shown in SEQ ID NO:23 and SEQ ID NO:24, respectively. In an embodiment, a 4-1BB agonist comprises V_{H} and V_{L} regions that are each at least 98% identical to the sequences shown in SEQ ID NO:23 and SEQ ID NO:24, respectively. In an embodiment, a 4-1BB agonist comprises V_{H} and V_{L} regions that are each at least 97% identical to the sequences shown in SEQ ID NO:23 and SEQ ID NO:24, respectively. In an embodiment, a 4-1BB agonist comprises V_{H} and V_{L} regions that are each at least 96% identical to the sequences shown in SEQ ID NO:23 and SEQ ID NO:24, respectively. In an embodiment, a 4-1BB agonist comprises V_{H} and V_{L} regions that are each at least 95% identical to the sequences shown in SEQ ID NO:23 and SEQ ID NO:24, respectively. In an embodiment, a 4-1BB agonist comprises an scFv antibody comprising V_{H} and V_{L} regions that are each at least 99% identical to the sequences shown in SEQ ID NO:23 and SEQ ID NO:24.

In an embodiment, a 4-1BB agonist comprises heavy chain CDR1, CDR2 and CDR3 domains having the sequences set forth in SEQ ID NO:25, SEQ ID NO:26, and SEQ ID NO:27, respectively, and conservative amino acid substitutions thereof, and light chain CDR1, CDR2 and CDR3 domains having the sequences set forth in SEQ ID NO:28, SEQ ID NO:29, and SEQ ID NO:30, respectively, and conservative amino acid substitutions thereof.

In an embodiment, the 4-1BB agonist is a 4-1BB agonist biosimilar monoclonal antibody approved by drug regulatory authorities with reference to urelumab. In an embodiment, the biosimilar monoclonal antibody comprises an 4-1BB antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is urelumab. In some embodiments, the one or more post-translational modifications are selected from one or more of: glycosylation, oxidation, deamidation, and truncation. In some embodiments, the biosimilar is a 4-1BB agonist antibody authorized or submitted for authorization, wherein the 4-1BB agonist antibody is provided in a formulation which differs from the formulations of a reference medicinal product or reference biological product, wherein the reference medicinal product or reference biological product is urelumab. The 4-1BB agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is urelumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is urelumab.

TABLE 5
Amino acid sequences for 4-1BB agonist antibodies related to urelumab.

| Identifier | Sequence (One-Letter Amino Acid Symbols) |
|---|---|
| SEQ ID NO: 21 | QVQLQQWGAG LLKPSETLSL TCAVYGGSFS GYYWSWIRQS PEKGLEWIGE INHGGYVTYN 60 |
| heavy chain for | PSLESRVTIS VDTSKNQFSL KLSSVTAADT AVYYCARDYG PGNYDWYFDL WGRGTLVTVS 120 |
| urelumab | SASTKGPSVF PLAPCSRSTS ESTAALGCLV KDYFPEPVTV SWNSGALTSG VHTFPAVLQS 180 |
| | SGLYSLSSVV TVPSSSLGTK TYTCNVDHKP SNTKVDKRVE SKYGPPCPPC PAPEFLGGPS 240 |
| | VFLFPPKPKD TLMISRTPEV TCVVVDVSQE DPEVQFNWYV DGVEVHNAKT KPREEQFNST 300 |
| | YRVVSVLTVL HQDWLNGKEY KCKVSNKGLP SSIEKTISKA KGQPREPQVY TLPPSQEEMT 360 |
| | KNQVSLTCLV KGFYPSDIAV EWESNGQPEN NYKTTPPVLD SDGSFFLYSR LTVDKSRWQE 420 |
| | GNVFSCSVMH EALHNHYTQK SLSLSLGK 448 |
| SEQ ID NO: 22 | EIVLTQSPAT LSLSPGERAT LSCRASQSVS SYLAWYQQKP GQAPRLLIYD ASNRATGIPA 60 |
| light chain for | RFSGSGSGTD FTLTISSLEP EDFAVYYCQQ RSNWPPALTF CGGTKVEIKR TVAAPSVFIF 120 |
| urelumab | PPSDEQLKSG TASVVCLLNN FYPREAKVQW KVDNALQSGN SQESVTEQDS KDSTYSLSST 180 |
| | LTLSKADYEK HKVYACEVTH QGLSSPVTKS FNRGEC 216 |
| SEQ ID NO: 23 | MKHLWFFLLL VAAPRWVLSQ VQLQQWGAGL LKPSETLSLT CAVYGGSFSG YYWSWIRQSP 60 |
| variable heavy | EKGLEWIGEI NHGGYVTYNP SLESRVTISV DTSKNQFSLK LSSVTAADTA VYYCARDYGP 120 |
| chain for | |
| urelumab | |
| SEQ ID NO: 24 | MEAPAQLLFL LLLWLPDTTG EIVLTQSPAT LSLSPGERAT LSCRASQSVS SYLAWYQQKP 60 |
| variable light | GQAPRLLIYD ASNRATGIPA RFSGSGSGTD FTLTISSLEP EDFAVYYCQQ 110 |
| chain for | |
| urelumab | |
| SEQ ID NO: 25 | GYYWS 5 |
| heavy chain CDR1 | |
| for urelumab | |
| SEQ ID NO: 26 | EINHGGYVTY NPSLES 16 |
| heavy chain CDR2 | |
| for urelumab | |
| SEQ ID NO: 27 | DYGPGNYDWY FDL 13 |
| heavy chain CDR3 | |
| for urelumab | |
| SEQ ID NO: 28 | RASQSVSSYL A 11 |
| light chain CDR1 | |
| for urelumab | |
| SEQ ID NO: 29 | DASNRAT 7 |
| light chain CDR2 | |
| for urelumab | |
| SEQ ID NO: 30 | QQRSDWPPAL T 11 |
| light chain CDR3 | |
| for urelumab | |

In an embodiment, the 4-1BB agonist is selected from the group consisting of 1D8, 3Elor, 4B4 (BioLegend 309809), H4-1BB-M127 (BD Pharmingen 552532), BBK2 (Thermo Fisher MS621PABX), 145501 (Leinco Technologies B591), the antibody produced by cell line deposited as ATCC No. HB-11248 and disclosed in U.S. Pat. No. 6,974,863, 5F4 (BioLegend 31 1503), C65-485 (BD Pharmingen 559446), antibodies disclosed in U.S. Patent Application Publication No. US 2005/0095244, antibodies disclosed in U.S. Pat. No. 7,288,638 (such as 20H4.9-IgGl (BMS-663031)), antibodies disclosed in U.S. Pat. No. 6,887,673 (such as 4E9 or BMS-554271), antibodies disclosed in U.S. Pat. No. 7,214,493, antibodies disclosed in U.S. Pat. No. 6,303,121, antibodies disclosed in U.S. Pat. No. 6,569,997, antibodies disclosed in U.S. Pat. No. 6,905,685 (such as 4E9 or BMS-554271), antibodies disclosed in U.S. Pat. No. 6,362,325 (such as 1D8 or BMS-469492; 3H3 or BMS-469497; or 3E1), antibodies disclosed in U.S. Pat. No. 6,974,863 (such as 53A2); antibodies disclosed in U.S. Pat. No. 6,210,669 (such as 1D8, 3B8, or 3E1), antibodies described in U.S. Pat. No. 5,928,893, antibodies disclosed in U.S. Pat. No. 6,303,121, antibodies disclosed in U.S. Pat. No. 6,569,997, antibodies disclosed in International Patent Application Publication Nos. WO 2012/177788, WO 2015/119923, and WO 2010/042433, and fragments, derivatives, conjugates, variants, or biosimilars thereof, wherein the disclosure of each of the foregoing patents or patent application publications is incorporated by reference here.

In an embodiment, the 4-1BB agonist is a 4-1BB agonistic fusion protein described in International Patent Application Publication Nos. WO 2008/025516 A1, WO 2009/007120 A1, WO 2010/003766 A1, WO 2010/010051 A1, and WO 2010/078966 A1; U.S. Patent Application Publication Nos. US 2011/0027218 A1, US 2015/0126709 A1, US 2011/0111494 A1, US 2015/0110734 A1, and US 2015/0126710 A1; and U.S. Pat. Nos. 9,359,420, 9,340,599, 8,921,519, and 8,450,460, the disclosures of which are incorporated by reference herein.

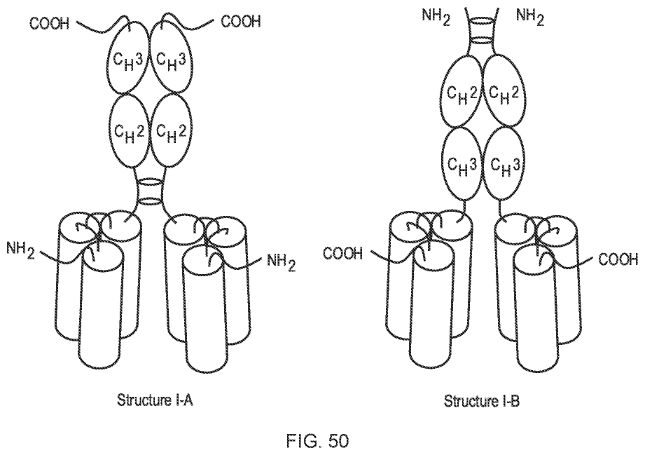

In an embodiment, the 4-1BB agonist is a 4-1BB agonistic fusion protein as depicted in Structure I-A (C-terminal Fc-antibody fragment fusion protein) or Structure I-B (N-terminal Fc-antibody fragment fusion protein) of FIG. 50, or a fragment, derivative, conjugate, variant, or biosimilar thereof. In structures I-A and I-B, the cylinders refer to individual polypeptide binding domains. Structures I-A and I-B comprise three linearly-linked TNFRSF binding domains derived from e.g., 4-1BBL or an antibody that binds 4-1BB, which fold to form a trivalent protein, which is then linked to a second triavelent protein through IgG1-Fc (including C_{H}3 and C_{H}2 domains) is then used to link two of the trivalent proteins together through disulfide bonds (small elongated ovals), stabilizing the structure and providing an agonists capable of bringing together the intracellular signaling domains of the six receptors and signaling proteins to form a signaling complex. The TNFRSF binding domains denoted as cylinders may be scFv domains comprising, e.g., a V_{H} and a V_{L} chain connected by a linker that may comprise hydrophilic residues and Gly and Ser sequences for flexibility, as well as Glu and Lys for solubility. Any scFv domain design may be used, such as those described in de Marco, Microbial Cell Factories, 2011, 10, 44; Ahmad, et al., Clin. & Dev. Immunol. 2012, 980250; Monnier, et al., Antibodies, 2013, 2, 193-208; or in references incorporated elsewhere herein. Fusion protein structures of this form are described in U.S. Pat. Nos. 9,359,420, 9,340,599, 8,921,519, and 8,450,460, the disclosures of which are incorporated by reference herein.

Amino acid sequences for the other polypeptide domains of structure I-A are given in Table 6. The Fc domain preferably comprises a complete constant domain (amino acids 17-230 of SEQ ID NO:31) the complete hinge domain (amino acids 1-16 of SEQ ID NO:31) or a portion of the hinge domain (e.g., amino acids 4-16 of SEQ ID NO:31). Preferred linkers for connecting a C-terminal Fc-antibody may be selected from the embodiments given in SEQ ID NO:32 to SEQ ID NO:41, including linkers suitable for fusion of additional polypeptides.

TABLE 6
Amino acid sequences for TNFRSF fusion proteins, including
4-1BB fusion proteins, with C-terminal Fc-antibody
fragment fusion protein design (structure I-A).

| Identifier | Sequence (One-Letter Amino Acid Symbols) |
|---|---|
| SEQ ID NO: 31 | KSCDKTHTCP PCPAPELLGG PSVFLFPPKP KDTLMISRTP EVTCVVVDVS HEDPEVKFNW 60 |
| Fc domain | YVDGVEVHNA KTKPREEQYN STYRVVSVLT VLHQDWLNGK EYKCKVSNKA LPAPIEKTIS 120 |
| | KAKGQPREPQ VYTLPPSREE MTKNQVSLTC LVKGFYPSDI AVEWESNGQP ENNYKTTPPV 180 |
| | LDSDGSFFLY SKLTVDKSRW QQGNVFSCSV MHEALHNHYT QKSLSLSPGK 230 |
| SEQ ID NO: 32 | GGPGSSKSCD KTHTCPPCPA PE 22 |
| linker | |
| SEQ ID NO: 33 | GGSGSSKSCD KTHTCPPCPA PE 22 |
| linker | |
| SEQ ID NO: 34 | GGPGSSSSSS SKSCDKTHTC PPCPAPE 27 |
| linker | |
| SEQ ID NO: 35 | GGSGSSSSSS SKSCDKTHTC PPCPAPE 27 |
| linker | |
| SEQ ID NO: 36 | GGPGSSSSSS SSSKSCDKTH TCPPCPAPE 29 |
| linker | |
| SEQ ID NO: 37 | GGSGSSSSSS SSSKSCDKTH TCPPCPAPE 29 |
| linker | |
| SEQ ID NO: 38 | GGPGSSGSGS SDKTHTCPPC PAPE 24 |
| linker | |
| SEQ ID NO: 39 | GGPGSSGSGS DKTHTCPPCP APE 23 |
| linker | |
| SEQ ID NO: 40 | GGPSSSGSDK THTCPPCPAP E 21 |
| linker | |
| SEQ ID NO: 41 | GGSSSSSSSS GSDKTHTCPP CPAPE 25 |
| linker | |

Amino acid sequences for the other polypeptide domains of structure I-B are given in Table 7. If an Fc antibody fragment is fused to the N-terminus of an TNRFSF fusion protein as in structure I-B, the sequence of the Fc module is preferably that shown in SEQ ID NO:42, and the linker sequences are preferably selected from those embodiments set forth in SED ID NO:43 to SEQ ID NO:45.

TABLE 7
Amino acid sequences for TNFRSF fusion proteins, including
4-1BB fusion proteins, with N-terminal Fc-antibody fragment
fusion protein design (structure I-B).

| Identifier | Sequence (One-Letter Amino Acid Symbols) |
|---|---|
| SEQ ID NO: 42 | METDTLLLWV LLLWVPAGNG DKTHTCPPCP APELLGGPSV FLFPPKPKDT LMISRTPEVT 60 |
| Fc domain | CVVVDVSHED PEVKFNWYVD GVEVHNAKTK PREEQYNSTY RVVSVLTVLH QDWLNGKEYK 120 |
| | CKVSNKALPA PIEKTISKAK GQPREPQVYT LPPSREEMTK NQVSLTCLVK GFYPSDIAVE 180 |
| | WESNGQPENN YKTTPPVLDS DGSFFLYSKL TVDKSRWQQG NVFSCSVMHE ALHNHYTQKS 240 |
| | LSLSPG 246 |
| SEQ ID NO: 43 | SGSGSGSGSG S 11 |
| linker | |
| SEQ ID NO: 44 | SSSSSSGSGS GS 12 |
| linker | |
| SEQ ID NO: 45 | SSSSSSGSGS GSGSGS 16 |
| linker | |

In an embodiment, a 4-1BB agonist fusion protein according to structures I-A or I-B comprises one or more 4-1BB binding domains selected from the group consisting of a variable heavy chain and variable light chain of utomilumab, a variable heavy chain and variable light chain of urelumab, a variable heavy chain and variable light chain of utomilumab, a variable heavy chain and variable light chain selected from the variable heavy chains and variable light chains described in Table 8, any combination of a variable heavy chain and variable light chain of the foregoing, and fragments, derivatives, conjugates, variants, and biosimilars thereof.

In an embodiment, a 4-1BB agonist fusion protein according to structures I-A or I-B comprises one or more 4-1BB binding domains comprising a 4-1BBL sequence. In an embodiment, a 4-1BB agonist fusion protein according to structures I-A or I-B comprises one or more 4-1BB binding domains comprising a sequence according to SEQ ID NO:46. In an embodiment, a 4-1BB agonist fusion protein according to structures I-A or I-B comprises one or more 4-1BB binding domains comprising a soluble 4-1BBL sequence. In an embodiment, a 4-1BB agonist fusion protein according to structures I-A or I-B comprises one or more 4-1BB binding domains comprising a sequence according to SEQ ID NO:47.

In an embodiment, a 4-1BB agonist fusion protein according to structures I-A or I-B comprises one or more 4-1BB binding domains that is a scFv domain comprising V_{H} and V_{L} regions that are each at least 95% identical to the sequences shown in SEQ ID NO:13 and SEQ ID NO:14, respectively, wherein the V_{H} and V_{L} domains are connected by a linker. In an embodiment, a 4-1BB agonist fusion protein according to structures I-A or I-B comprises one or more 4-1BB binding domains that is a scFv domain comprising V_{H} and V_{L} regions that are each at least 95% identical to the sequences shown in SEQ TD NO:23 and SEQ TD NO:24, respectively, wherein the V_{H} and V_{L} domains are connected by a linker. In an embodiment, a 4-1BB agonist fusion protein according to structures I-A or I-B comprises one or more 4-1BB binding domains that is a scFv domain comprising V_{H} and V_{L} regions that are each at least 950 identical to the V_{H} and V_{L} sequences given in Table 8, wherein the V_{H} and V_{L} domains are connected by a linker.

TABLE 8
Additional polypeptide domains useful as 4-1BB binding domains in fusion proteins
or as scFv 4-1BB agonist antibodies.

| Identifier | Sequence (One-Letter Amino Acid Symbols) | |
|---|---|---|
| SEQ ID NO: 46 | MEYASDASLD PEAPWPPAPR ARACRVLPWA LVAGLLLLLL LAAACAVFLA CPWAVSGARA | 60 |
| 4-1BBL | SPGSAASPRL REGPELSPDD PAGLLDLRQG MFAQLVAQNV LLIDGPLSWY SDPGLAGVSL | 120 |
| | TGGLSYKEDT KELVVAKAGV YYVFFQLELR RVVAGEGSGS VSLALHLQPL RSAAGAAALA | 100 |
| | LTVDLPPASS EARNSAFGFQ GRLLHLSAGQ RLGVHLHTEA RARHAWQLTQ GATVLGLFRV | 240 |
| | TPEIPAGLPS PRSE | 254 |
| SEQ ID NO: 47 | LRQGMFAQLV AQNVLLIDGP LSWYSDPGLA GVSLTGGLSY KEDTKELVVA KAGVYYVFFQ | 60 |
| 4-1BBL soluble | LELRRVVAGE GSGSVSLALH LQPLRSAAGA AALALTVDLP PASSEARNSA FGFQGRLLHL | 120 |
| domain | SAGQRLGVHL HTEARARHAW QLTQGATVLG LFRVTPEIPA GLPSPRSE | 168 |
| SEQ ID NO: 48 | QVQLQQPGAE LVKPGASVKL SCKASGYTFS SYWMHWVKQR PGQVLEWIGE INPGNGHTNY | 60 |
| variable heavy | NEKFKSKATL TVDKSSSTAY MQLSSLTSED SAVYYCARSF TTARGFAYWG QGTLVTVS | 118 |
| chain for 4B4-1- | | |
| 1 version 1 | | |
| SEQ ID NO: 49 | DIVMTQSPAT QSVTPGDRVS LSCRASQTIS DYLHWYQQKS HESPRLLIKY ASQSISGIPS | 60 |
| variable light | RFSGSGSGSD FTLSINSVEP EDVGVYYCQD GHSFPPTFGG GTKLEIK | 107 |
| chain for 4B4-1- | | |
| 1 version 1 | | |
| SEQ ID NO: 50 | QVQLQQPGAE LVKPGASVKL SCKASGYTFS SYWMHWVKQR PGQVLEWIGE INPGNGHTNY | 60 |
| variable heavy | NEKFKSKATL TVDKSSSTAY MQLSSLTSED SAVYYCARSF TTARGFAYWG QGTLVTVSA | 119 |
| chain for 4B4-1- | | |
| 1 version 2 | | |
| SEQ ID NO: 51 | DIVMTQSPAT QSVTPGDRVS LSCRASQTIS DYLHWYQQKS HESPRLLIKY ASQSISGIPS | 60 |
| variable light | RFSGSGSGSD FTLSINSVEP EDVGVYYCQD GHSFPPTFGG GTKLEIKR | 108 |
| chain for 4B4-1- | | |
| 1 version 2 | | |
| SEQ ID NO: 52 | MDWTWRILFL VAAATGAHSE VQLVESGGGL VQPGGSLRLS CAASGFTFSD YWMSWVRQAP | 60 |
| variable heavy | GKGLEWVADI KNDGSYTNYA PSLTNRFTIS RDNAKNSLYL QMNSLRAEDT AVYYCARELT | 120 |
| chain for H39E3- | | |
| 2 | | |
| SEQ ID NO: 53 | MEAPAQLLFL LLLWLPDTTG DIVMTQSPDS LAVSLGERAT INCKSSQSLL SSGNQKNYL | 60 |
| variable light | WYQQKPGQPP KLLIYYASTR QSGVPDRFSG SGSGTDFTLT ISSLQAEDVA | 110 |
| chain for H39E3- | | |
| 2 | | |

In an embodiment, the 4-inn agonist is a 4-inn agonistic single-chain fusion polypeptide comprising (i) a first soluble 4-inn binding domain, (ii) a first peptide linker, (iii) a second soluble 4-inn binding domain, (iv) a second peptide linker, and (v) a third soluble 4-inn binding domain, further comprising an additional domain at the N-terminal and/or C-terminal end, and wherein the additional domain is a Fab or Fc fragment domain. In an embodiment, the 4-1BB agonist is a 4-1BB agonistic single-chain fusion polypeptide comprising (i) a first soluble 4-1BB binding domain, (ii) a first peptide linker, (iii) a second soluble 4-1BB binding domain, (iv) a second peptide linker, and (v) a third soluble 4-1BB binding domain, further comprising an additional domain at the N-terminal and/or C-terminal end, wherein the additional domain is a Fab or Fc fragment domain, wherein each of the soluble 4-1BB domains lacks a stalk region (which contributes to trimerisation and provides a certain distance to the cell membrane, but is not part of the 4-1BB binding domain) and the first and the second peptide linkers independently have a length of 3-8 amino acids.

In an embodiment, the 4-1BB agonist is a 4-1BB agonistic single-chain fusion polypeptide comprising (i) a first soluble tumor necrosis factor (TNF) superfamily cytokine domain, (ii) a first peptide linker, (iii) a second soluble TNF superfamily cytokine domain, (iv) a second peptide linker, and (v) a third soluble TNF superfamily cytokine domain, wherein each of the soluble TNF superfamily cytokine domains lacks a stalk region and the first and the second peptide linkers independently have a length of 3-8 amino acids, and wherein each TNF superfamily cytokine domain is a 4-1BB binding domain.

In an embodiment, the 4-1BB agonist is a 4-1BB agonistic scFv antibody comprising any of the foregoing V_{H} domains linked to any of the foregoing V_{L} domains.

In an embodiment, the 4-1BB agonist agonist is BPS Bioscience 4-1BB agonist antibody catalog no. 79097-2, commercially available from BPS Bioscience, San Diego, CA, USA. In an embodiment, the 4-1BB agonist agonist is Creative Biolabs 4-1BB agonist antibody catalog no. MOM-18179, commercially available from Creative Biolabs, Shirley, NY, USA.

### OX40 (CD134) Agonists

In an embodiment, the TNFRSF agonist is an OX40 (CD134) agonist. The OX40 agonist may be any OX40 binding molecule known in the art. The OX40 binding molecule may be a monoclonal antibody or fusion protein capable of binding to human or mammalian OX40. The OX40 agonists or OX40 binding molecules may comprise an immunoglobulin heavy chain of any isotype (e.g., IgG, IgE, IgM, IgD, IgA, and IgY), class (e.g., IgG1, IgG2, IgG3, IgG4, IgA1 and IgA2) or subclass of immunoglobulin molecule. The OX40 agonist or OX40 binding molecule may have both a heavy and a light chain. As used herein, the term binding molecule also includes antibodies (including full length antibodies), monoclonal antibodies (including full length monoclonal antibodies), polyclonal antibodies, multispecific antibodies (e.g., bispecific antibodies), human, humanized or chimeric antibodies, and antibody fragments, e.g., Fab fragments, F(ab′) fragments, fragments produced by a Fab expression library, epitope-binding fragments of any of the above, and engineered forms of antibodies, e.g., scFv molecules, that bind to OX40. In an embodiment, the OX40 agonist is an antigen binding protein that is a fully human antibody. In an embodiment, the OX40 agonist is an antigen binding protein that is a humanized antibody. In some embodiments, OX40 agonists for use in the presently disclosed methods and compositions include anti-OX40 antibodies, human anti-OX40 antibodies, mouse anti-OX40 antibodies, mammalian anti-OX40 antibodies, monoclonal anti-OX40 antibodies, polyclonal anti-OX40 antibodies, chimeric anti-OX40 antibodies, anti-OX40 adnectins, anti-OX40 domain antibodies, single chain anti-OX40 fragments, heavy chain anti-OX40 fragments, light chain anti-OX40 fragments, anti-OX40 fusion proteins, and fragments, derivatives, conjugates, variants, or biosimilars thereof. In a preferred embodiment, the OX40 agonist is an agonistic, anti-OX40 humanized or fully human monoclonal antibody (i.e., an antibody derived from a single cell line).

In a preferred embodiment, the OX40 agonist or OX40 binding molecule may also be a fusion protein. OX40 fusion proteins comprising an Fc domain fused to OX40L are described, for example, in Sadun, et al., J. Immunother. 2009, 182, 1481-89. In a preferred embodiment, a multimeric OX40 agonist, such as a trimeric or hexameric OX40 agonist (with three or six ligand binding domains), may induce superior receptor (OX40L) clustering and internal cellular signaling complex formation compared to an agonistic monoclonal antibody, which typically possesses two ligand binding domains. Trimeric (trivalent) or hexameric (or hexavalent) or greater fusion proteins comprising three TNFRSF binding domains and IgG1-Fc and optionally further linking two or more of these fusion proteins are described, e.g., in Gieffers, et al., Mol. Cancer Therapeutics 2013, 12, 2735-47.

Agonistic OX40 antibodies and fusion proteins are known to induce strong immune responses. Curti, et al., Cancer Res. 2013, 73, 7189-98. In a preferred embodiment, the OX40 agonist is a monoclonal antibody or fusion protein that binds specifically to OX40 antigen in a manner sufficient to reduce toxicity. In some embodiments, the OX40 agonist is an agonistic OX40 monoclonal antibody or fusion protein that abrogates antibody-dependent cellular toxicity (ADCC), for example NK cell cytotoxicity. In some embodiments, the OX40 agonist is an agonistic OX40 monoclonal antibody or fusion protein that abrogates antibody-dependent cell phagocytosis (ADCP). In some embodiments, the OX40 agonist is an agonistic OX40 monoclonal antibody or fusion protein that abrogates complement-dependent cytotoxicity (CDC). In some embodiments, the OX40 agonist is an agonistic OX40 monoclonal antibody or fusion protein which abrogates Fc region functionality.

In some embodiments, the OX40 agonists are characterized by binding to human OX40 (SEQ ID NO:54) with high affinity and agonistic activity. In an embodiment, the OX40 agonist is a binding molecule that binds to human OX40 (SEQ ID NO:54). In an embodiment, the OX40 agonist is a binding molecule that binds to murine OX40 (SEQ ID NO:55). The amino acid sequences of OX40 antigen to which an OX40 agonist or binding molecule binds are summarized in Table 9.

TABLE 9
Amino acid sequences of OX40 antigens.

| Identifier | Sequence (One-Letter Amino Acid Symbols) | |
|---|---|---|
| SEQ ID NO: 54 | MCVGARRLGR GPCAALLLLG LGLSTVTGLH CVGDTYPSND RCCHECRPGN GMVSRCSRSQ | 60 |
| human OX40 | NTVCRPCGPG FYNDVVSSKP CKPCTWCNLR SGSERKQLCT ATQDTVCRCR AGTQPLDSYK | 120 |
| (Homo sapiens) | PGVDCAPCPP GHFSPGDNQA CKPWTNCTLA GKHTLQPASN SSDAICEDRD PPATQPQETQ | 180 |
| | GPPARPITVQ PTEAWPRTSQ GPSTRPVEVP GGRAVAAILG LGLVLGLLGP LAILLALYLL | 240 |
| | RRDQRLPPDA HKPPGGGSFR TPIQEEQADA HSTLAKI | 277 |
| SEQ ID NO: 55 | MYVWVQQPTA LLLLGLTLGV TARRLNCVKH TYPSGHKCCR ECQPGHGMVS RCDHTRDTLC | 60 |
| murine OX40 | HPCETGFYNE AVNYDTCKQC TQCNHRSGSE LKQNCTPTQD TVCRCRPGTQ PRQDSGYKLG | 120 |
| (Mus musculus) | VDCVPCPPGH FSPGNNQACK PWTNCTLSGK QTRHPASDSL DAVCEDRSLL ATLLWETQRP | 180 |
| | TFRPTTVQST TVWPRTSELP SPPTLVTPEG PAFAVLLGLG LGLLAPLTVL LALYLLRKAW | 240 |
| | RLPNTPKPCW GNSFRTPIQE EHTDAHFTLA KI | 272 |

In some embodiments, the compositions, processes and methods described include a OX40 agonist that binds human or murine OX40 with a K_{D} of about 100 pM or lower, binds human or murine OX40 with a K_{D} of about 90 pM or lower, binds human or murine OX40 with a K_{D} of about 80 pM or lower, binds human or murine OX40 with a K_{D} of about 70 pM or lower, binds human or murine OX40 with a K_{D} of about 60 pM or lower, binds human or murine OX40 with a K_{D} of about 50 pM or lower, binds human or murine OX40 with a K_{D} of about 40 pM or lower, or binds human or murine OX40 with a K_{D} of about 30 pM or lower.

In some embodiments, the compositions, processes and methods described include a OX40 agonist that binds to human or murine OX40 with a k_{assoc} of about 7.5×10⁵ l/M·s or faster, binds to human or murine OX40 with a k_{assoc} of about 7.5×10⁵ l/M·s or faster, binds to human or murine OX40 with a k_{assoc} of about 8×10⁵ l/M·s or faster, binds to human or murine OX40 with a k_{assoc} of about 8.5×10⁵ l/M·s or faster, binds to human or murine OX40 with a k_{assoc} of about 9×10⁵ l/M·s or faster, binds to human or murine OX40 with a k_{assoc} of about 9.5×10⁵ l/M·s or faster, or binds to human or murine OX40 with a k_{assoc} of about 1×10⁶ l/M·s or faster.

In some embodiments, the compositions, processes and methods described include a OX40 agonist that binds to human or murine OX40 with a k_{dissoc} of about 2×10⁻⁵ l/s or slower, binds to human or murine OX40 with a k_{dissoc} of about 2.1×10⁻⁵ l/s or slower, binds to human or murine OX40 with a k_{dissoc} of about 2.2×10⁻⁵ l/s or slower, binds to human or murine OX40 with a k_{dissoc} of about 2.3×10⁻⁵ l/s or slower, binds to human or murine OX40 with a k_{dissoc} of about 2.4×10⁻⁵ l/s or slower, binds to human or murine OX40 with a k_{dissoc} of about 2.5×10⁻⁵ l/s or slower, binds to human or murine OX40 with a k_{dissoc} of about 2.6×10⁻⁵ l/s or slower or binds to human or murine OX40 with a k_{dissoc} of about 2.7×10⁻⁵ l/s or slower, binds to human or murine OX40 with a k_{dissoc} of about 2.8×10⁻⁵ l/s or slower, binds to human or murine OX40 with a k_{dissoc} of about 2.9×10⁻⁵ l/s or slower, or binds to human or murine OX40 with a k_{dissoc} of about 3×10⁻⁵ l/s or slower.

In some embodiments, the compositions, processes and methods described include OX40 agonist that binds to human or murine OX40 with an IC₅₀ of about 10 nM or lower, binds to human or murine OX40 with an IC₅₀ of about 9 nM or lower, binds to human or murine OX40 with an IC₅₀ of about 8 nM or lower, binds to human or murine OX40 with an IC₅₀ of about 7 nM or lower, binds to human or murine OX40 with an IC₅₀ of about 6 nM or lower, binds to human or murine OX40 with an IC₅₀ of about 5 nM or lower, binds to human or murine OX40 with an IC₅₀ of about 4 nM or lower, binds to human or murine OX40 with an IC₅₀ of about 3 nM or lower, binds to human or murine OX40 with an IC₅₀ of about 2 nM or lower, or binds to human or murine OX40 with an IC₅₀ of about 1 nM or lower.

In some embodiments, the OX40 agonist is tavolixizumab, also known as MEDI0562 or MEDI-0562. Tavolixizumab is available from the MedImmune subsidiary of AstraZeneca, Inc. Tavolixizumab is immunoglobulin G1-kappa, anti-[Homo sapiens TNFRSF4 (tumor necrosis factor receptor (TNFR) superfamily member 4, OX40, CD134)], humanized and chimeric monoclonal antibody. The amino acid sequences of tavolixizumab are set forth in Table 10. Tavolixizumab comprises N-glycosylation sites at positions 301 and 301″, with fucosylated complex bi-antennary CHO-type glycans; heavy chain intrachain disulfide bridges at positions 22-95 (V_{H}-V_{L}), 148-204 (C_{H}1-C_{L}), 265-325 (C_{H}2) and 371-429 (C_{H}3) (and at positions 22″-95″, 148″-204″, 265″-325″, and 371″-429″); light chain intrachain disulfide bridges at positions 23′-88′ (V_{H}-V_{L}) and 134′-194′ (C_{H}1-C_{L}) (and at positions 23′″-88′″ and 134′″-194′″); interchain heavy chain-heavy chain disulfide bridges at positions 230-230″ and 233-233″; and interchain heavy chain-light chain disulfide bridges at 224-214′ and 224″-214′″. Current clinical trials of tavolixizumab in a variety of solid tumor indications include U.S. National Institutes of Health clinicaltrials.gov identifiers NCT02318394 and NCT02705482.

In an embodiment, a OX40 agonist comprises a heavy chain given by SEQ ID NO:56 and a light chain given by SEQ ID NO:57. In an embodiment, a OX40 agonist comprises heavy and light chains having the sequences shown in SEQ ID NO:56 and SEQ ID NO:57, respectively, or antigen binding fragments, Fab fragments, single-chain variable fragments (scFv), variants, or conjugates thereof. In an embodiment, a OX40 agonist comprises heavy and light chains that are each at least 99% identical to the sequences shown in SEQ ID NO:56 and SEQ ID NO:57, respectively. In an embodiment, a OX40 agonist comprises heavy and light chains that are each at least 98% identical to the sequences shown in SEQ ID NO:56 and SEQ ID NO:57, respectively. In an embodiment, a OX40 agonist comprises heavy and light chains that are each at least 97% identical to the sequences shown in SEQ ID NO:56 and SEQ ID NO:57, respectively. In an embodiment, a OX40 agonist comprises heavy and light chains that are each at least 96% identical to the sequences shown in SEQ ID NO:56 and SEQ ID NO:57, respectively. In an embodiment, a OX40 agonist comprises heavy and light chains that are each at least 95% identical to the sequences shown in SEQ ID NO:56 and SEQ ID NO:57, respectively.

In an embodiment, the OX40 agonist comprises the heavy and light chain CDRs or variable regions (VRs) of tavolixizumab. In an embodiment, the OX40 agonist heavy chain variable region (V_{H}) comprises the sequence shown in SEQ ID NO:58, and the OX40 agonist light chain variable region (V_{L}) comprises the sequence shown in SEQ ID NO:59, and conservative amino acid substitutions thereof. In an embodiment, a OX40 agonist comprises V_{H} and V_{L} regions that are each at least 99% identical to the sequences shown in SEQ ID NO:58 and SEQ ID NO:59, respectively. In an embodiment, a OX40 agonist comprises V_{H} and V_{L} regions that are each at least 98% identical to the sequences shown in SEQ ID NO:58 and SEQ ID NO:59, respectively. In an embodiment, a OX40 agonist comprises V_{H} and V_{L} regions that are each at least 97% identical to the sequences shown in SEQ ID NO:58 and SEQ ID NO:59, respectively. In an embodiment, a OX40 agonist comprises V_{H} and V_{L} regions that are each at least 96% identical to the sequences shown in SEQ ID NO:58 and SEQ ID NO:59, respectively. In an embodiment, a OX40 agonist comprises V_{H} and V_{L} regions that are each at least 95% identical to the sequences shown in SEQ ID NO:58 and SEQ ID NO:59, respectively. In an embodiment, an OX40 agonist comprises an scFv antibody comprising V_{H} and V_{L} regions that are each at least 99% identical to the sequences shown in SEQ ID NO:58 and SEQ ID NO:59.

In an embodiment, a OX40 agonist comprises heavy chain CDR1, CDR2 and CDR3 domains having the sequences set forth in SEQ ID NO:60, SEQ ID NO:61, and SEQ ID NO:62, respectively, and conservative amino acid substitutions thereof, and light chain CDR1, CDR2 and CDR3 domains having the sequences set forth in SEQ ID NO:63, SEQ ID NO:64, and SEQ ID NO:65, respectively, and conservative amino acid substitutions thereof.

In an embodiment, the OX40 agonist is a OX40 agonist biosimilar monoclonal antibody approved by drug regulatory authorities with reference to tavolixizumab. In an embodiment, the biosimilar monoclonal antibody comprises an OX40 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is tavolixizumab. In some embodiments, the one or more post-translational modifications are selected from one or more of: glycosylation, oxidation, deamidation, and truncation. In some embodiments, the biosimilar is a OX40 agonist antibody authorized or submitted for authorization, wherein the OX40 agonist antibody is provided in a formulation which differs from the formulations of a reference medicinal product or reference biological product, wherein the reference medicinal product or reference biological product is tavolixizumab. The OX40 agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is tavolixizumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is tavolixizumab.

TABLE 10
Amino acid sequences for OX40 agonist antibodies related to tavolixizumab.

| Identifier | Sequence (One-Letter Amino Acid Symbols) | |
|---|---|---|
| SEQ ID NO: 56 | QVQLQESGPG LVKPSQTLSL TCAVYGGSFS SGYWNWIRKH PGKGLEYIGY ISYNGITYHN | 60 |
| heavy chain for | PSLKSRITIN RDTSKNQYSL QLNSVTPEDT AVYYCARYKY DYDGGHAMDY WGQGTLVTVS | 120 |
| tavolixizumab | SASTKGPSVF PLAPSSKSTS GGTAALGCLV KDYFPEPVTV SWNSGALTSG VHTFPAVLQS | 180 |
| | SGLYSLSSVV TVPSSSLGTQ TYICNVNHKP SNTKVDKRVE PKSCDKTHTC PPCPAPELLG | 240 |
| | GPSVFLFPPK PKDTLMISRT PEVTCVVVDV SHEDPEVKFN WYVDGVEVHN AKTKPREEQY | 300 |
| | NSTYRVVSVL TVLHQDWLNG KEYKCKVSNK ALPAPIEKTI SKAKGQPREP QVYTLPPSRE | 360 |
| | EMTKNQVSLT CLVKGFYPSD IAVEWESNGQ PENNYKTTPP VLDSDGSFFL YSKLTVDKSR | 420 |
| | WQQGNVFSCS VMHEALHNHY TQKSLSLSPG K | 451 |
| SEQ ID NO: 57 | DIQMTQSPSS LSASVGDRVT ITCRASQDIS NYLNWYQQKP GKAPKLLIYY TSKLHSGVPS | 60 |
| light chain for | RFSGSGSGTD YTLTISSLQP EDFATYYCQQ GSALPWTFGQ GTKVEIKRTV AAPSVFIFPP | 120 |
| tavolixizumab | SDEQLKSGTA SVVCLLNNFY PREAKVQWKV DNALQSGNSQ ESVTEQDSKD STYSLSSTLT | 180 |
| | LSKADYEKHK VYACEVTHQG LSSPVTKSFN RGEC | 214 |
| SEQ ID NO: 58 | QVQLQESGPG LVKPSQTLSL TCAVYGGSFS SGYWNWIRKH PGKGLEYIGY ISYNGITYHN | 60 |
| heavy chain | PSLKSRITIN RDTSKNQYSL QLNSVTPEDT AVYYCARYKY DYDGGHAMDY WGQGTLVT | 118 |
| variable region | | |
| for | | |
| tavolixizumab | | |
| SEQ ID NO: 59 | DIQMTQSPSS LSASVGDRVT ITCRASQDIS NYLNWYQQKP GKAPKLLIYY TSKLHSGVPS | 60 |
| light chain | RFSGSGSGTD YTLTISSLQP EDFATYYCQQ GSALPWTFGQ GTKVEIKR | 108 |
| variable region | | |
| for | | |
| tavolixizumab | | |
| SEQ ID NO: 60 | GSFSSGYWN | 9 |
| heavy chain CDR1 | | |
| for | | |
| tavolixizumab | | |
| SEQ ID NO: 61 | YIGYISYNGI TYH | 13 |
| heavy chain CDR2 | | |
| for | | |
| tavolixizumab | | |
| SEQ ID NO: 62 | RYKYDYDGGH AMDY | 14 |
| heavy chain CDR3 | | |
| for | | |
| tavolixizumab | | |
| SEQ ID NO: 63 | QDISNYLN | 8 |
| light chain CDR1 | | |
| for | | |
| tavolixizumab | | |
| SEQ ID NO: 64 | LLIYYTSKLH S | 11 |
| light chain CDR2 | | |
| for | | |
| tavolixizumab | | |
| SEQ ID NO: 65 | QQGSALPW | 8 |
| light chain CDR3 | | |
| for | | |
| tavolixizumab | | |

In some embodiments, the OX40 agonist is 11D4, which is a fully human antibody available from Pfizer, Inc. The preparation and properties of 11D4 are described in U.S. Pat. Nos. 7,960,515; 8,236,930; and 9,028,824, the disclosures of which are incorporated by reference herein. The amino acid sequences of 11D4 are set forth in Table 11.

In an embodiment, a OX40 agonist comprises a heavy chain given by SEQ ID NO:66 and a light chain given by SEQ ID NO:67. In an embodiment, a OX40 agonist comprises heavy and light chains having the sequences shown in SEQ ID NO:66 and SEQ ID NO:67, respectively, or antigen binding fragments, Fab fragments, single-chain variable fragments (scFv), variants, or conjugates thereof. In an embodiment, a OX40 agonist comprises heavy and light chains that are each at least 99% identical to the sequences shown in SEQ ID NO:66 and SEQ ID NO:67, respectively. In an embodiment, a OX40 agonist comprises heavy and light chains that are each at least 98% identical to the sequences shown in SEQ ID NO:66 and SEQ ID NO:67, respectively. In an embodiment, a OX40 agonist comprises heavy and light chains that are each at least 97% identical to the sequences shown in SEQ ID NO:66 and SEQ ID NO:67, respectively. In an embodiment, a OX40 agonist comprises heavy and light chains that are each at least 96% identical to the sequences shown in SEQ ID NO:66 and SEQ ID NO:67, respectively. In an embodiment, a OX40 agonist comprises heavy and light chains that are each at least 95% identical to the sequences shown in SEQ ID NO:66 and SEQ ID NO:67, respectively.

In an embodiment, the OX40 agonist comprises the heavy and light chain CDRs or variable regions (VRs) of 11D4. In an embodiment, the OX40 agonist heavy chain variable region (V_{H}) comprises the sequence shown in SEQ ID NO:68, and the OX40 agonist light chain variable region (V_{L}) comprises the sequence shown in SEQ ID NO:69, and conservative amino acid substitutions thereof. In an embodiment, a OX40 agonist comprises V_{H} and V_{L} regions that are each at least 99% identical to the sequences shown in SEQ ID NO:68 and SEQ ID NO:69, respectively. In an embodiment, a OX40 agonist comprises V_{H} and V_{L} regions that are each at least 98% identical to the sequences shown in SEQ ID NO:68 and SEQ ID NO:69, respectively. In an embodiment, a OX40 agonist comprises V_{H} and V_{L} regions that are each at least 97% identical to the sequences shown in SEQ ID NO:68 and SEQ ID NO:69, respectively. In an embodiment, a OX40 agonist comprises V_{H} and V_{L} regions that are each at least 96% identical to the sequences shown in SEQ ID NO:68 and SEQ ID NO:69, respectively. In an embodiment, a OX40 agonist comprises V_{H} and V_{L} regions that are each at least 95% identical to the sequences shown in SEQ ID NO:68 and SEQ ID NO:69, respectively.

In an embodiment, a OX40 agonist comprises heavy chain CDR1, CDR2 and CDR3 domains having the sequences set forth in SEQ ID NO:70, SEQ ID NO:71, and SEQ ID NO:72, respectively, and conservative amino acid substitutions thereof, and light chain CDR1, CDR2 and CDR3 domains having the sequences set forth in SEQ ID NO:73, SEQ ID NO:74, and SEQ ID NO:75, respectively, and conservative amino acid substitutions thereof.

In an embodiment, the OX40 agonist is a OX40 agonist biosimilar monoclonal antibody approved by drug regulatory authorities with reference to 11D4. In an embodiment, the biosimilar monoclonal antibody comprises an OX40 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 11D4. In some embodiments, the one or more post-translational modifications are selected from one or more of: glycosylation, oxidation, deamidation, and truncation. In some embodiments, the biosimilar is a OX40 agonist antibody authorized or submitted for authorization, wherein the OX40 agonist antibody is provided in a formulation which differs from the formulations of a reference medicinal product or reference biological product, wherein the reference medicinal product or reference biological product is 11D4. The OX40 agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 11D4. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 11D4.

TABLE 11
Amino acid sequences for OX40 agonist antibodies related to 11D4.

| Identifier | Sequence (One-Letter Amino Acid Symbols) | |
|---|---|---|
| SEQ ID NO: 66 | EVQLVESGGG LVQPGGSLRL SCAASGFTFS SYSMNWVRQA PGKGLEWVSY ISSSSSTIDY | 60 |
| heavy chain for | ADSVKGRFTI SRDNAKNSLY LQMNSLRDED TAVYYCARES GWYLFDYWGQ GTLVTVSSAS | 120 |
| 11D4 | TKGPSVFPLA PCSRSTSEST AALGCLVKDY FPEPVTVSWN SGALTSGVHT FPAVLQSSGL | 160 |
| | YSLSSVVTVP SSNFGTQTYT CNVDHKPSNT KVDKTVERKC CVECPPCPAP PVAGPSVFLF | 240 |
| | PPKPKDTLMI SRTPEVTCVV VDVSHEDPEV QFNWYVDGVE VHNAKTKPRE EQFNSTFRVV | 300 |
| | SVLTVVHQDW LNGKEYKCKV SNKGLPAPIE KTISKTKGQP REPQVYTLPP SREEMTKNQV | 360 |
| | SLTCLVKGFY PSDIAVEWES NGQPENNYKT TPPMLDSDGS FFLYSKLTVD KSRWQQGNVF | 420 |
| | SCSVMHEALH NHYTQKSLSL SPGK | 444 |
| SEQ ID NO: 67 | DIQMTQSPSS LSASVGDRVT ITCRASQGIS SWLAWYQQKP EKAPKSLIYA ASSLQSGVPS | 60 |
| light chain for | RFSGSGSGTD FTLTISSLQP EDFATYYCQQ YNSYPPTFGG GTKVEIKRTV AAPSVFIFPP | 120 |
| 11D4 | SDEQLKSGTA SVVCLLNNFY PREAKVQWKV DNALQSGNSQ ESVTEQDSKD STYSLSSTLT | 180 |
| | LSKADYEKHK VYACEVTHQG LSSPVTKSFN RGEC | 214 |
| SEQ ID NO: 68 | EVQLVESGGG LVQPGGSLRL SCAASGFTFS SYSMNWVRQA PGKGLEWVSY ISSSSSTIDY | 60 |
| heavy chain | ADSVKGRFTI SRDNAKNSLY LQMNSLRDED TAVYYCARES GWYLFDYWGQ GTLVTVSS | 118 |
| variable region | | |
| for 11D4 | | |
| SEQ ID NO: 69 | DIQMTQSPSS LSASVGDRVT ITCRASQGIS SWLAWYQQKP EKAPKSLIYA ASSLQSGVPS | 60 |
| light chain | RFSGSGSGTD FTLTISSLQP EDFATYYCQQ YNSYPPTFGG GTKVEIK | 107 |
| variable region | | |
| for 11D4 | | |
| SEQ ID NO: 70 | SYSMN | 5 |
| heavy chain CDR1 | | |
| for 11D4 | | |
| SEQ ID NO: 71 | YISSSSSTID YADSVKG | 17 |
| heavy chain CDR2 | | |
| for 11D4 | | |
| SEQ ID NO: 72 | ESGWYLFDY | 9 |
| heavy chain CDR3 | | |
| for 11D4 | | |
| SEQ ID NO: 73 | RASQGISSWL A | 11 |
| light chain CDR1 | | |
| for 11D4 | | |
| SEQ ID NO: 74 | AASSLQS | 7 |
| light chain CDR2 | | |
| for 11D4 | | |
| SEQ ID NO: 75 | QQYNSYPPT | 9 |
| light chain CDR3 | | |
| for 11D4 | | |

In some embodiments, the OX40 agonist is 18D38, which is a fully human antibody available from Pfizer, Inc. The preparation and properties of 18D38 are described in U.S. Pat. Nos. 7,960,515; 8,236,930; and 9,028,824, the disclosures of which are incorporated by reference herein. The amino acid sequences of 18D38 are set forth in Table 12.

In an embodiment, a OX40 agonist comprises a heavy chain given by SEQ TD NO:76 and a light chain given by SEQ TD NO:77. In an embodiment, a OX40 agonist comprises heavy and light chains having the sequences shown in SEQ TD NO:76 and SEQ ID NO:77, respectively, or antigen binding fragments, Fab fragments, single-chain variable fragments (scFv), variants, or conjugates thereof. In an embodiment, a OX40 agonist comprises heavy and light chains that are each at least 9900 identical to the sequences shown in SEQ TD NO:76 and SEQ ID NO:77, respectively. In an embodiment, a OX40 agonist comprises heavy and light chains that are each at least 98% identical to the sequences shown in SEQ ID NO:76 and SEQ ID NO:77, respectively. In an embodiment, a OX40 agonist comprises heavy and light chains that are each at least 97% identical to the sequences shown in SEQ ID NO:76 and SEQ ID NO:77, respectively. In an embodiment, a OX40 agonist comprises heavy and light chains that are each at least 96% identical to the sequences shown in SEQ ID NO:76 and SEQ ID NO:77, respectively. In an embodiment, a OX40 agonist comprises heavy and light chains that are each at least 95% identical to the sequences shown in SEQ ID NO:76 and SEQ ID NO:77, respectively.

In an embodiment, the OX40 agonist comprises the heavy and light chain CDRs or variable regions (VRs) of 18D8. In an embodiment, the OX40 agonist heavy chain variable region (V_{H}) comprises the sequence shown in SEQ ID NO:78, and the OX40 agonist light chain variable region (V_{L}) comprises the sequence shown in SEQ ID NO:79, and conservative amino acid substitutions thereof. In an embodiment, a OX40 agonist comprises V_{H} and V_{L} regions that are each at least 99% identical to the sequences shown in SEQ ID NO:78 and SEQ ID NO:79, respectively. In an embodiment, a OX40 agonist comprises V_{H} and V_{L} regions that are each at least 98% identical to the sequences shown in SEQ ID NO:78 and SEQ ID NO:79, respectively. In an embodiment, a OX40 agonist comprises V_{H} and V_{L} regions that are each at least 97% identical to the sequences shown in SEQ ID NO:78 and SEQ ID NO:79, respectively. In an embodiment, a OX40 agonist comprises V_{H} and V_{L} regions that are each at least 96% identical to the sequences shown in SEQ ID NO:78 and SEQ ID NO:79, respectively. In an embodiment, a OX40 agonist comprises V_{H} and V_{L} regions that are each at least 95% identical to the sequences shown in SEQ ID NO:78 and SEQ ID NO:79, respectively.

In an embodiment, a OX40 agonist comprises heavy chain CDR1, CDR2 and CDR3 domains having the sequences set forth in SEQ ID NO:80, SEQ ID NO:81, and SEQ ID NO:82, respectively, and conservative amino acid substitutions thereof, and light chain CDR1, CDR2 and CDR3 domains having the sequences set forth in SEQ ID NO:83, SEQ ID NO:84, and SEQ ID NO:85, respectively, and conservative amino acid substitutions thereof.

In an embodiment, the OX40 agonist is a OX40 agonist biosimilar monoclonal antibody approved by drug regulatory authorities with reference to 18D8. In an embodiment, the biosimilar monoclonal antibody comprises an OX40 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 18D8. In some embodiments, the one or more post-translational modifications are selected from one or more of: glycosylation, oxidation, deamidation, and truncation. In some embodiments, the biosimilar is a OX40 agonist antibody authorized or submitted for authorization, wherein the OX40 agonist antibody is provided in a formulation which differs from the formulations of a reference medicinal product or reference biological product, wherein the reference medicinal product or reference biological product is 18D8. The OX40 agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 18D8. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 18D8.

TABLE 12
Amino acid sequences for OX40 agonist antibodies related to 18D8.

| Identifier | Sequence (One-Letter Amino Acid Symbols) | |
|---|---|---|
| SEQ ID NO: 76 | EVQLVESGGG LVQPGRSLRL SCAASGFTFD DYAMHWVRQA PGKGLEWVSG ISWNSGSIGY | 60 |
| heavy chain for | ADSVKGRFTI SRDNAKNSLY LQMNSLRAED TALYYCAKDQ STADYYFYYG MDVWGQGTTV | 120 |
| 18D8 | TVSSASTKGP SVFPLAPCSR STSESTAALG CLVKDYFPEP VTVSWNSGAL TSGVHTFPAV | 180 |
| | LQSSGLYSLS SVVTVPSSNF GTQTYTCNVD HKPSNTKVDK TVERKCCVEC PPCPAPPVAG | 240 |
| | PSVFLFPPKP KDTLMISRTP EVTCVVVDVS HEDPEVQFNW YVDGVEVHNA KTKPREEQFN | 300 |
| | STFRVVSVLT VVHQDWLNGK EYKCKVSNKG LPAPIEKTIS KTKGQPREPQ VYTLPPSREE | 360 |
| | MTKNQVSLTC LVKGFYPSDI AVEWESNGQP ENNYKTTPPM LDSDGSFFLY SKLTVDKSRW | 420 |
| | QQGNVFSCSV MHEALHNHYT QKSLSLSPGK | 450 |
| SEQ ID NO: 77 | EIVVTQSPAT LSLSPGERAT LSCRASQSVS SYLAWYQQKP GQAPRLLIYD ASNRATGIPA | 60 |
| light chain for | RFSGSGSGTD FTLTISSLEP EDFAVYYCQQ RSNWPTFGQG TKVEIKRTVA APSVFIFPPS | 120 |
| 18D8 | DEQLKSGTAS VVCLLNNFYP REAKVQWKVD NALQSGNSQE SVTEQDSKDS TYSLSSTLTL | 180 |
| | SKADYEKHKV YACEVTHQGL SSPVTKSFNR GEC | 213 |
| SEQ ID NO: 78 | EVQLVESGGG LVQPGRSLRL SCAASGFTFD DYAMHWVRQA PGKGLEWVSG ISWNSGSIGY | 60 |
| heavy chain | ADSVKGRFTI SRDNAKNSLY LQMNSLRAED TALYYCAKDQ STADYYFYYG MDVWGQGTTV | 120 |
| variable region | TVSS | 124 |
| for 18D8 | | |
| SEQ ID NO: 79 | EIVVTQSPAT LSLSPGERAT LSCRASQSVS SYLAWYQQKP GQAPRLLIYD ASNRATGIPA | 60 |
| light chain | RFSGSGSGTD FTLTISSLEP EDFAVYYCQQ RSNWPTFGQG TKVEIK | 106 |
| variable region | | |
| for 18D8 | | |
| SEQ ID NO: 80 | DYAMH | 5 |
| heavy chain CDR1 | | |
| for 18D8 | | |
| SEQ ID NO: 81 | GISWNSGSIG YADSVKG | 17 |
| heavy chain CDR2 | | |
| for 18D8 | | |
| SEQ ID NO: 82 | DQSTADYYFY YGMDV | 15 |
| heavy chain CDR3 | | |
| for 18D8 | | |
| SEQ ID NO: 83 | RASQSVSSYL A | 11 |
| light chain CDR1 | | |
| for 18D8 | | |
| SEQ ID NO: 84 | DASNRAT | 7 |
| light chain CDR2 | | |
| for 18D8 | | |
| SEQ ID NO: 85 | QQRSNWPT | 8 |
| light chain CDR3 | | |
| for 18D8 | | |

In some embodiments, the OX40 agonist is Hu119-122, which is a humanized antibody available from GlaxoSmithKline plc. The preparation and properties of Hu119-122 are described in U.S. Pat. Nos. 9,006,399 and 9,163,085, and in International Patent Publication No. WO 2012/027328, the disclosures of which are incorporated by reference herein. The amino acid sequences of Hu119-122 are set forth in Table 13.

In an embodiment, the OX40 agonist comprises the heavy and light chain CDRs or variable regions (VRs) of Hu119-122. In an embodiment, the OX40 agonist heavy chain variable region (V_{H}) comprises the sequence shown in SEQ ID NO:86, and the OX40 agonist light chain variable region (V_{L}) comprises the sequence shown in SEQ ID NO:87, and conservative amino acid substitutions thereof. In an embodiment, a OX40 agonist comprises V_{H} and V_{L} regions that are each at least 99% identical to the sequences shown in SEQ ID NO:86 and SEQ ID NO:87, respectively. In an embodiment, a OX40 agonist comprises V_{H} and V_{L} regions that are each at least 98% identical to the sequences shown in SEQ ID NO:86 and SEQ ID NO:87, respectively. In an embodiment, a OX40 agonist comprises V_{H} and V_{L} regions that are each at least 97% identical to the sequences shown in SEQ ID NO:86 and SEQ ID NO:87, respectively. In an embodiment, a OX40 agonist comprises V_{H} and V_{L} regions that are each at least 96% identical to the sequences shown in SEQ ID NO:86 and SEQ ID NO:87, respectively. In an embodiment, a OX40 agonist comprises V_{H} and V_{L} regions that are each at least 95% identical to the sequences shown in SEQ ID NO:86 and SEQ ID NO:87, respectively.

In an embodiment, a OX40 agonist comprises heavy chain CDR1, CDR2 and CDR3 domains having the sequences set forth in SEQ ID NO:88, SEQ ID NO:89, and SEQ ID NO:90, respectively, and conservative amino acid substitutions thereof, and light chain CDR1, CDR2 and CDR3 domains having the sequences set forth in SEQ ID NO:91, SEQ ID NO:92, and SEQ ID NO:93, respectively, and conservative amino acid substitutions thereof.

In an embodiment, the OX40 agonist is a OX40 agonist biosimilar monoclonal antibody approved by drug regulatory authorities with reference to Hu119-122. In an embodiment, the biosimilar monoclonal antibody comprises an OX40 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is Hu119-122. In some embodiments, the one or more post-translational modifications are selected from one or more of: glycosylation, oxidation, deamidation, and truncation. In some embodiments, the biosimilar is a OX40 agonist antibody authorized or submitted for authorization, wherein the OX40 agonist antibody is provided in a formulation which differs from the formulations of a reference medicinal product or reference biological product, wherein the reference medicinal product or reference biological product is Hu119-122. The OX40 agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is Hu119-122. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is Hu119-122.

TABLE 13
Amino acid sequences for OX40 agonist antibodies related to Hu119-122.

| Identifier | Sequence (One-Letter Amino Acid Symbols) | |
|---|---|---|
| SEQ ID NO: 86 | EVQLVESGGG LVQPGGSLRL SCAASEYEFP SHDMSWVRQA PGKGLELVAA INSDGGSTYY | 60 |
| heavy chain | PDTMERRFTI SRDNAKNSLY LQMNSLRAED TAVYYCARHY DDYYAWFAYW GQGTMVTVSS | 120 |
| variable region | | |
| for Hu119-122 | | |
| SEQ ID NO: 87 | EIVLTQSPAT LSLSPGERAT LSCRASKSVS TSGYSYMHWY QQKPGQAPRL LIYLASNLES | 60 |
| light chain | GVPARFSGSG SGTDFTLTIS SLEPEDFAVY YCQHSRELPL TFGGGTKVEI K | 111 |
| variable region | | |
| for Hu119-122 | | |
| SEQ ID NO: 88 | SHDMS | 5 |
| heavy chain CDR1 | | |
| for Hu119-122 | | |
| SEQ ID NO: 89 | AINSDGGSTY YPDTMER | 17 |
| heavy chain CDR2 | | |
| for Hu119-122 | | |
| SEQ ID NO: 90 | HYDDYYAWFA Y | 11 |
| heavy chain CDR3 | | |
| for Hu119-122 | | |
| SEQ ID NO: 91 | RASKSVSTSG YSYMH | 15 |
| light chain CDR1 | | |
| for Hu119-122 | | |
| SEQ ID NO: 92 | LASNLES | 7 |
| light chain CDR2 | | |
| for Hu119-122 | | |
| SEQ ID NO: 93 | QHSRELPLT | 9 |
| light chain CDR3 | | |
| for Hu119-122 | | |

In some embodiments, the OX40 agonist is Hu106-222, which is a humanized antibody available from GlaxoSmithKline plc. The preparation and properties of Hu106-222 are described in U.S. Pat. Nos. 9,006,399 and 9,163,085, and in International Patent Publication No. WO 2012/027328, the disclosures of which are incorporated by reference herein. The amino acid sequences of Hu106-222 are set forth in Table 14.

In an embodiment, the OX40 agonist comprises the heavy and light chain CDRs or variable regions (VRs) of Hu106-222. In an embodiment, the OX40 agonist heavy chain variable region (V_{H}) comprises the sequence shown in SEQ ID NO:94, and the OX40 agonist light chain variable region (V_{L}) comprises the sequence shown in SEQ ID NO:95, and conservative amino acid substitutions thereof. In an embodiment, a OX40 agonist comprises V_{H} and V_{L} regions that are each at least 99% identical to the sequences shown in SEQ ID NO:94 and SEQ ID NO:95, respectively. In an embodiment, a OX40 agonist comprises V_{H} and V_{L} regions that are each at least 98% identical to the sequences shown in SEQ ID NO:94 and SEQ ID NO:95, respectively. In an embodiment, a OX40 agonist comprises V_{H} and V_{L} regions that are each at least 97% identical to the sequences shown in SEQ ID NO:94 and SEQ ID NO:95, respectively. In an embodiment, a OX40 agonist comprises V_{H} and V_{L} regions that are each at least 96% identical to the sequences shown in SEQ ID NO:94 and SEQ ID NO:95, respectively. In an embodiment, a OX40 agonist comprises V_{H} and V_{L} regions that are each at least 95% identical to the sequences shown in SEQ ID NO:94 and SEQ ID NO:95, respectively.

In an embodiment, a OX40 agonist comprises heavy chain CDR1, CDR2 and CDR3 domains having the sequences set forth in SEQ ID NO:96, SEQ ID NO:97, and SEQ ID NO:98, respectively, and conservative amino acid substitutions thereof, and light chain CDR1, CDR2 and CDR3 domains having the sequences set forth in SEQ ID NO:99, SEQ ID NO:100, and SEQ ID NO:101, respectively, and conservative amino acid substitutions thereof.

In an embodiment, the OX40 agonist is a OX40 agonist biosimilar monoclonal antibody approved by drug regulatory authorities with reference to Hu106-222. In an embodiment, the biosimilar monoclonal antibody comprises an OX40 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is Hu106-222. In some embodiments, the one or more post-translational modifications are selected from one or more of: glycosylation, oxidation, deamidation, and truncation. In some embodiments, the biosimilar is a OX40 agonist antibody authorized or submitted for authorization, wherein the OX40 agonist antibody is provided in a formulation which differs from the formulations of a reference medicinal product or reference biological product, wherein the reference medicinal product or reference biological product is Hu106-222. The OX40 agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is Hu106-222. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is Hu106-222.

TABLE 14
Amino acid sequences for OX40 agonist antibodies related to Hu106-222.

| Identifier | Sequence (One-Letter Amino Acid Symbols) | |
|---|---|---|
| SEQ ID NO: 94 | QVQLVQSGSE LKKPGASVKV SCKASGYTFT DYSMHWVRQA PGQGLKWMGW INTETGEPTY | 60 |
| heavy chain | ADDFKGRFVF SLDTSVSTAY LQISSLKAED TAVYYCANPY YDYVSYYAMD YWGQGTTVTV | 120 |
| variable region | SS | 122 |
| for Hu106-222 | | |
| SEQ ID NO: 95 | DIQMTQSPSS LSASVGDRVT ITCKASQDVS TAVAWYQQKP GKAPKLLIYS ASYLYTGVPS | 60 |
| light chain | RFSGSGSGTD FTFTISSLQP EDIATYYCQQ HYSTPRTFGQ GTKLEIK | 107 |
| variable region | | |
| for Hu106-222 | | |
| SEQ ID NO: 96 | DYSMH | 5 |
| heavy chain CDR1 | | |
| for Hu106-222 | | |
| SEQ ID NO: 97 | WINTETGEPT YADDFKG | 17 |
| heavy chain CDR2 | | |
| for Hu106-222 | | |
| SEQ ID NO: 98 | PYYDYVSYYA MDY | 13 |
| heavy chain CDR3 | | |
| for Hu106-222 | | |
| SEQ ID NO: 99 | KASQDVSTAV A | 11 |
| light chain CDR1 | | |
| for Hu106-222 | | |
| SEQ ID NO: 100 | SASYLYT | 7 |
| light chain CDR2 | | |
| for Hu106-222 | | |
| SEQ ID NO: 101 | QQHYSTPRT | 9 |
| light chain CDR3 | | |
| for Hu106-222 | | |

In some embodiments, the OX40 agonist antibody is MEDI6469 (also referred to as 9B12). MEDI6469 is a murine monoclonal antibody. Weinberg, et al., J. Immunother. 2006, 29, 575-585. In some embodiments the OX40 agonist is an antibody produced by the 9B12 hybridoma, deposited with Biovest Inc. (Malvern, MA, USA), as described in Weinberg, et al., J. Immunother. 2006, 29, 575-585, the disclosure of which is hereby incorporated by reference in its entirety. In some embodiments, the antibody comprises the CDR sequences of MEDI6469. In some embodiments, the antibody comprises a heavy chain variable region sequence and/or a light chain variable region sequence of MEDI6469.

In an embodiment, the OX40 agonist is L106 BD (Pharmingen Product #340420). In some embodiments, the OX40 agonist comprises the CDRs of antibody L106 (BD Pharmingen Product #340420). In some embodiments, the OX40 agonist comprises a heavy chain variable region sequence and/or a light chain variable region sequence of antibody L106 (BD Pharmingen Product #340420). In an embodiment, the OX40 agonist is ACT35 (Santa Cruz Biotechnology, Catalog #20073). In some embodiments, the OX40 agonist comprises the CDRs of antibody ACT35 (Santa Cruz Biotechnology, Catalog #20073). In some embodiments, the OX40 agonist comprises a heavy chain variable region sequence and/or a light chain variable region sequence of antibody ACT35 (Santa Cruz Biotechnology, Catalog #20073). In an embodiment, the OX40 agonist is the murine monoclonal antibody anti-mCD134/mOX40 (clone OX86), commercially available from InVivoMAb, BioXcell Inc, West Lebanon, NH.

In an embodiment, the OX40 agonist is selected from the OX40 agonists described in International Patent Application Publication Nos. WO 95/12673, WO 95/21925, WO 2006/121810, WO 2012/027328, WO 2013/028231, WO 2013/038191, and WO 2014/148895; European Patent Application EP 0672141; U.S. Patent Application Publication Nos. US 2010/136030, US 2014/377284, US 2015/190506, and US 2015/132288 (including clones 20E5 and 12H3); and U.S. Pat. Nos. 7,504,101, 7,550,140, 7,622,444, 7,696,175, 7,960,515, 7,961,515, 8,133,983, 9,006,399, and 9,163,085, the disclosure of each of which is incorporated herein by reference in its entirety.

In an embodiment, the OX40 agonist is an OX40 agonistic fusion protein as depicted in Structure I-A (C-terminal Fc-antibody fragment fusion protein) or Structure I-B (N-terminal Fc-antibody fragment fusion protein), or a fragment, derivative, conjugate, variant, or biosimilar thereof. The properties of structures I-A and I-B are described above and in U.S. Pat. Nos. 9,359,420, 9,340,599, 8,921,519, and 8,450,460, the disclosures of which are incorporated by reference herein. Amino acid sequences for the polypeptide domains of structure I-A are given in Table 6. The Fc domain preferably comprises a complete constant domain (amino acids 17-230 of SEQ ID NO:31) the complete hinge domain (amino acids 1-16 of SEQ ID NO:31) or a portion of the hinge domain (e.g., amino acids 4-16 of SEQ ID NO:31). Preferred linkers for connecting a C-terminal Fc-antibody may be selected from the embodiments given in SEQ ID NO:32 to SEQ ID NO:41, including linkers suitable for fusion of additional polypeptides. Likewise, amino acid sequences for the polypeptide domains of structure I-B are given in Table 7. If an Fc antibody fragment is fused to the N-terminus of an TNRFSF fusion protein as in structure I-B, the sequence of the Fc module is preferably that shown in SEQ ID NO:42, and the linker sequences are preferably selected from those embodiments set forth in SED ID NO:43 to SEQ ID NO:45.

In an embodiment, an OX40 agonist fusion protein according to structures I-A or I-B comprises one or more OX40 binding domains selected from the group consisting of a variable heavy chain and variable light chain of tavolixizumab, a variable heavy chain and variable light chain of 11D4, a variable heavy chain and variable light chain of 18D8, a variable heavy chain and variable light chain of Hu119-122, a variable heavy chain and variable light chain of Hu106-222, a variable heavy chain and variable light chain selected from the variable heavy chains and variable light chains described in Table 15, any combination of a variable heavy chain and variable light chain of the foregoing, and fragments, derivatives, conjugates, variants, and biosimilars thereof.

In an embodiment, an OX40 agonist fusion protein according to structures I-A or I-B comprises one or more OX40 binding domains comprising an OX40L sequence. In an embodiment, an OX40 agonist fusion protein according to structures I-A or I-B comprises one or more OX40 binding domains comprising a sequence according to SEQ ID NO:102. In an embodiment, an OX40 agonist fusion protein according to structures I-A or I-B comprises one or more OX40 binding domains comprising a soluble OX40L sequence. In an embodiment, a OX40 agonist fusion protein according to structures I-A or I-B comprises one or more OX40 binding domains comprising a sequence according to SEQ ID NO:103. In an embodiment, a OX40 agonist fusion protein according to structures I-A or I-B comprises one or more OX40 binding domains comprising a sequence according to SEQ ID NO:104.

In an embodiment, an OX40 agonist fusion protein according to structures I-A or I-B comprises one or more OX40 binding domains that is a scFv domain comprising V_{H} and V_{L} regions that are each at least 95% identical to the sequences shown in SEQ ID NO:58 and SEQ ID NO:59, respectively, wherein the V_{H} and V_{L} domains are connected by a linker. In an embodiment, an OX40 agonist fusion protein according to structures I-A or I-B comprises one or more OX40 binding domains that is a scFv domain comprising V_{H} and V_{L} regions that are each at least 95% identical to the sequences shown in SEQ ID NO:68 and SEQ ID NO:69, respectively, wherein the V_{H} and V_{L} domains are connected by a linker. In an embodiment, an OX40 agonist fusion protein according to structures I-A or I-B comprises one or more OX40 binding domains that is a scFv domain comprising V_{H} and V_{L} regions that are each at least 95% identical to the sequences shown in SEQ ID NO:78 and SEQ ID NO:79, respectively, wherein the V_{H} and V_{L} domains are connected by a linker. In an embodiment, an OX40 agonist fusion protein according to structures I-A or I-B comprises one or more OX40 binding domains that is a scFv domain comprising V_{H} and V_{L} regions that are each at least 95% identical to the sequences shown in SEQ ID NO:86 and SEQ ID NO:87, respectively, wherein the V_{H} and V_{L} domains are connected by a linker. In an embodiment, an OX40 agonist fusion protein according to structures I-A or I-B comprises one or more OX40 binding domains that is a scFv domain comprising V_{H} and V_{L} regions that are each at least 95% identical to the sequences shown in SEQ ID NO:94 and SEQ ID NO:95, respectively, wherein the V_{H} and V_{L} domains are connected by a linker. In an embodiment, an OX40 agonist fusion protein according to structures I-A or I-B comprises one or more OX40 binding domains that is a scFv domain comprising V_{H} and V_{L} regions that are each at least 95% identical to the V_{H} and V_{L} sequences given in Table 15, wherein the V_{H} and V_{L} domains are connected by a linker.

TABLE 15
Additional polypeptide domains useful as OX40 binding domains in fusion
proteins (e.g., structures I-A and I-B) or as scFv OX40 agonist antibodies.

| Identifier | Sequence (One-Letter Amino Acid Symbols) | |
|---|---|---|
| SEQ ID NO: 102 | MERVQPLEEN VGNAARPRFE RNKLLLVASV IQGLGLLLCF TYICLHFSAL QVSHRYPRIQ | 60 |
| OX40L | SIKVQFTEYK KEKGFILTSQ KEDEIMKVQN NSVIINCDGF YLISLKGYFS QEVNISLHYQ | 120 |
| | KDEEPLFQLK KVRSVNSLMV ASLTYKDKVY LNVTTDNTSL DDFHVNGGEL ILIHQNPGEF | 180 |
| | CVL | 183 |
| SEQ ID NO: 103 | SHRYPRIQSI KVQFTEYKKE KGFILTSQKE DEIMKVQNNS VIINCDGFYL ISLKGYFSQE | 60 |
| OX40L soluble | VNISLHYQKD EEPLFQLKKV RSVNSLMVAS LTYKDKVYLN VTTDNTSLDD FHVNGGELIL | 120 |
| domain | IHQNPGEFCV L | 131 |
| SEQ ID NO: 104 | YPRIQSIKVQ FTEYKKEKGF ILTSQKEDEI MKVQNNSVII NCDGFYLISL KGYFSQEVNI | 60 |
| OX40L soluble | SLHYQKDEEP LFQLKKVRSV NSLMVASLTY KDKVYLNVTT DNTSLDDFHV NGGELILIHQ | 120 |
| domain | NPGEFCVL | 128 |
| (alternative) | | |
| SEQ ID NO: 105 | EVQLVESGGG LVQPGGSLRL SCAASGFTFS NYTMNWVRQA PGKGLEWVSA ISGSGGSTYY | 60 |
| variable heavy | ADSVXGRFTI SRDNSKNTLY LQMNSLRAED TAVYYCAKDR YSQVHYALDY WGQGTLVTVS | 120 |
| chain for 008 | | |
| SEQ ID NO: 106 | DIVMTQSPDS LPVTPGEPAS ISCRSSQSLL HSNGYNYLDW YLQKAGQSPQ LLIYLGSNRA | 60 |
| variable light | SGVPDRFSGS GSGTDFTLKI SRVEAEDVGV YYCQQYYNHP TTFGQGTK | 108 |
| chain for 008 | | |
| SEQ ID NO: 107 | EVQLVESGGG VVQPGRSLRL SCAASGFTFS DYTMNWVRQA PGKGLEWVSS ISGGSTYYAD | 60 |
| variable heavy | SRKGRFTISR DNSKNTLYLQ MNNLRAEDTA VYYCARDRYF RQQNAFDYWG QGTLVTVSSA | 120 |
| chain for 011 | | |
| SEQ ID NO: 108 | DIVMTQSPDS LPVTPGEPAS ISCRSSQSLL HSNGYNYLDW YLQKAGQSPQ LLIYLGSNRA | 60 |
| variable light | SGVPDRFSGS GSGTDFTLKI SRVEAEDVGV YYCQQYYNHP TTFGQGTK | 108 |
| chain for 011 | | |
| SEQ ID NO: 109 | EVQLVESGGG LVQPRGSLRL SCAASGFTFS SYAMNWVRQA PGKGLEWVAV ISYDGSNKYY | 60 |
| variable heavy | ADSVKGRFTI SRDNSKNTLY LQMNSLRAED TAVYYCAKDR YITLPNALDY WGQGTLVTVS | 120 |
| chain for 021 | | |
| SEQ ID NO: 110 | DIQMTQSPVS LPVTPGEPAS ISCRSSQSLL HSNGYNYLDW YLQKPGQSPQ LLIYLGSNRA | 60 |
| variable light | SGVPDRFSGS GSGTDFTLKI SRVEAEDVGV YYCQQYKSNP PTFGQGTK | 108 |
| chain for 021 | | |
| SEQ ID NO: 111 | EVQLVESGGG LVHPGGSLRL SCAGSGFTFS SYAMHWVRQA PGKGLEWVSA IGTGGGTYYA | 60 |
| variable heavy | DSVMGRFTIS RDNSKNTLYL QMNSLRAEDT AVYYCARYDN VMGLYWFDYW GQGTLVTVSS | 120 |
| chain for 023 | | |
| SEQ ID NO: 112 | EIVLTQSPAT LSLSPGERAT LSCRASQSVS SYLAWYQQKP GQAPRLLIYD ASNRATGIPA | 60 |
| variable light | RFSGSGSGTD FTLTISSLEP EDFAVYYCQQ RSNWPPAFGG GTKVEIKR | 106 |
| chain for 023 | | |
| SEQ ID NO: 113 | EVQLQQSGPE LVKPGASVKM SCKASGYTFT SYVMHWVKQK PGQGLEWIGY INPYNDGTKY | 60 |
| heavy chain | NEKFKGKATL TSDKSSSTAY MELSSLTSED SAVYYCANYY GSSLSMDYWG QGTSVTVSS | 119 |
| variable region | | |
| SEQ ID NO: 114 | DIQMTQTTSS LSASLGDRVT ISCRASQDIS NYLNWYQQKP DGTVKLLIYY TSRLHSGVPS | 60 |
| light chain | RFSGSGSGTD YSLTISNLEQ EDIATYFCQQ GNTLPWTFGG GTKLEIKR | 108 |
| variable region | | |
| SEQ ID NO: 115 | EVQLQQSGPE LVKPGASVKI SCKTSGYTFK DYTMHWVKQS HGKSLEWIGG IYPNNGGSTY | 60 |
| heavy chain | NQNFKDKATL TVDKSSSTAY MEFRSLTSED SAVYYCARMG YHGPHLDFDV WGAGTTVTVS | 120 |
| variable region | P | 121 |
| SEQ ID NO: 116 | DIVMTQSHKF MSTSLGDRVS ITCKASQDVG AAVAWYQQKP GQSPKLLIYW ASTRHTGVPD | 60 |
| light chain | RFTGGGSGTD FTLTISNVQS EDLTDYFCQQ YINYPLTFGG GTKLEIKR | 108 |
| variable region | | |
| SEQ ID NO: 117 | QIQLVQSGPE LKKPGETVKI SCKASGYTFT DYSMHWVKQA PGKGLKWMGW INTETGEPTY | 60 |
| heavy chain | ADDFKGRFAF SLETSASTAY LQINNLKNED TATYFCANPY YDYVSYYAMD YWGHGTSVTV | 120 |
| variable region | SS | 122 |
| of humanized | | |
| antibody | | |
| SEQ ID NO: 118 | QVQLVQSGSE LKKPGASVKV SCKASGYTFT DYSMHWVRQA PGQGLKWMGW INTETGEPTY | 60 |
| heavy chain | ADDFKGRFVF SLDTSVSTAY LQISSLKAED TAVYYCANPY YDYVSYYAMD YWGQGTTVTV | 120 |
| variable region | SS | 122 |
| of humanized | | |
| antibody | | |
| SEQ ID NO: 119 | DIVMTQSHKF MSTSVRDRVS ITCKASQDVS TAVAWYQQKP GQSPKLLIYS ASYLYTGVPD | 60 |
| light chain | RFTGSGSGTD FTFTISSVQA EDLAVYYCQQ HYSTPRTFGG GTKLEIK | 107 |
| variable region | | |
| of humanized | | |
| antibody | | |
| SEQ ID NO: 120 | DIVMTQSHKF MSTSVRDRVS ITCKASQDVS TAVAWYQQKP GQSPKLLIYS ASYLYTGVPD | 60 |
| light chain | RFTGSGSGTD FTFTISSVQA EDLAVYYCQQ HYSTPRTFGG GTKLEIK | 107 |
| variable region | | |
| of humanized | | |
| antibody | | |
| SEQ ID NO: 121 | EVQLVESGGG LVQPGESLKL SCESNEYEFP SHDMSWVRKT PEKRLELVAA INSDGGSTYY | 60 |
| heavy chain | PDTMERRFII SRDNTKKTLY LQMSSLRSED TALYYCARHY DDYYAWFAYW GQGTLVTVSA | 120 |
| variable region | | |
| of humanized | | |
| antibody | | |
| SEQ ID NO: 122 | EVQLVESGGG LVQPGGSLRL SCAASEYEFP SHDMSWVRQA PGKGLELVAA INSDGGSTYY | 60 |
| heavy chain | PDTMERRFTI SRDNAKNSLY LQMNSLRAED TAVYYCARHY DDYYAWFAYW GQGTMVTVSS | 120 |
| variable region | | |
| of humanized | | |
| antibody | | |
| SEQ ID NO: 123 | DIVLTQSPAS LAVSLGQRAT ISCRASKSVS TSGYSYMHWY QQKPGQPPKL LIYLASNLES | 60 |
| light chain | GVPARFSGSG SGTDFTLNIH PVEEEDAATY YCQHSRELPL TFGAGTKLEL K | 111 |
| variable region | | |
| of humanized | | |
| antibody | | |
| SEQ ID NO: 124 | EIVLTQSPAT LSLSPGERAT LSCRASKSVS TSGYSYMHWY QQKPGQAPRL LIYLASNLES | 60 |
| light chain | GVPARFSGSG SGTDFTLTIS SLEPEDFAVY YCQHSRELPL TFGGGTKVEI K | 111 |
| variable region | | |
| of humanized | | |
| antibody | | |
| SEQ ID NO: 125 | MYLGLNYVFI VFLLNGVQSE VKLEESGGGL VQPGGSMKLS CAASGFTFSD AWMDWVRQSP | 60 |
| heavy chain | EKGLEWVAEI RSKANNHATY YAESVNGRFT ISRDDSKSSV YLQMNSLRAE DTGIYYCTWG | 120 |
| variable region | EVFYFDYWGQ GTTLTVSS | 138 |
| SEQ ID NO: 126 | MRPSIQFLGL LLFWLHGAQC DIQMTQSPSS LSASLGGKVT ITCKSSQDIN KYIAWYQHKP | 60 |
| light chain | GKGPRLLIHY TSTLQPGIPS RFSGSGSGRD YSFSISNLEP EDIATYYCLQ YDNLLTFGAG | 120 |
| variable region | TKLELK | 126 |

In an embodiment, the OX40 agonist is a OX40 agonistic single-chain fusion polypeptide comprising (i) a first soluble OX40 binding domain, (ii) a first peptide linker, (iii) a second soluble OX40 binding domain, (iv) a second peptide linker, and (v) a third soluble OX40 binding domain, further comprising an additional domain at the N-terminal and/or C-terminal end, and wherein the additional domain is a Fab or Fc fragment domain. In an embodiment, the OX40 agonist is a OX40 agonistic single-chain fusion polypeptide comprising (i) a first soluble OX40 binding domain, (ii) a first peptide linker, (iii) a second soluble OX40 binding domain, (iv) a second peptide linker, and (v) a third soluble OX40 binding domain, further comprising an additional domain at the N-terminal and/or C-terminal end, wherein the additional domain is a Fab or Fc fragment domain wherein each of the soluble OX40 binding domains lacks a stalk region (which contributes to trimerisation and provides a certain distance to the cell membrane, but is not part of the OX40 binding domain) and the first and the second peptide linkers independently have a length of 3-8 amino acids.

In an embodiment, the OX40 agonist is an OX40 agonistic single-chain fusion polypeptide comprising (i) a first soluble tumor necrosis factor (TNF) superfamily cytokine domain, (ii) a first peptide linker, (iii) a second soluble TNF superfamily cytokine domain, (iv) a second peptide linker, and (v) a third soluble TNF superfamily cytokine domain, wherein each of the soluble TNF superfamily cytokine domains lacks a stalk region and the first and the second peptide linkers independently have a length of 3-8 amino acids, and wherein the TNF superfamily cytokine domain is an OX40 binding domain.

In some embodiments, the OX40 agonist is MEDI6383. MEDI6383 is an OX40 agonistic fusion protein and can be prepared as described in U.S. Pat. No. 6,312,700, the disclosure of which is incorporated by reference herein.

In an embodiment, the OX40 agonist is an OX40 agonistic scFv antibody comprising any of the foregoing V_{H} domains linked to any of the foregoing V_{L} domains.

In an embodiment, the OX40 agonist is Creative Biolabs OX40 agonist monoclonal antibody MOM-18455, commercially available from Creative Biolabs, Inc., Shirley, NY, USA.

In an embodiment, the OX40 agonist is OX40 agonistic antibody clone Ber-ACT35 commercially available from BioLegend, Inc., San Diego, CA, USA.

### CD27 Agonists

In an embodiment, the TNFRSF agonist is a CD27 agonist. The CD27 agonist may be any CD27 binding molecule known in the art. The CD27 binding molecule may be a monoclonal antibody or fusion protein capable of binding to human or mammalian CD27. The CD27 agonists or CD27 binding molecules may comprise an immunoglobulin heavy chain of any isotype (e.g., IgG, IgE, IgM, IgD, IgA, and IgY), class (e.g., IgG1, IgG2, IgG3, IgG4, IgA1 and IgA2) or subclass of immunoglobulin molecule. The CD27 agonist or CD27 binding molecule may have both a heavy and a light chain. As used herein, the term binding molecule also includes antibodies (including full length antibodies), monoclonal antibodies (including full length monoclonal antibodies), polyclonal antibodies, multispecific antibodies (e.g., bispecific antibodies), human, humanized or chimeric antibodies, and antibody fragments, e.g., Fab fragments, F(ab′) fragments, fragments produced by a Fab expression library, epitope-binding fragments of any of the above, and engineered forms of antibodies, e.g., scFv molecules, that bind to CD27. In an embodiment, the CD27 agonist is an antigen binding protein that is a fully human antibody. In an embodiment, the CD27 agonist is an antigen binding protein that is a humanized antibody. In some embodiments, CD27 agonists for use in the presently disclosed methods and compositions include anti-CD27 antibodies, human anti-CD27 antibodies, mouse anti-CD27 antibodies, mammalian anti-CD27 antibodies, monoclonal anti-CD27 antibodies, polyclonal anti-CD27 antibodies, chimeric anti-CD27 antibodies, anti-CD27 adnectins, anti-CD27 domain antibodies, single chain anti-CD27 fragments, heavy chain anti-CD27 fragments, light chain anti-CD27 fragments, anti-CD27 fusion proteins, and fragments, derivatives, conjugates, variants, or biosimilars thereof. In a preferred embodiment, the CD27 agonist is an agonistic, anti-CD27 humanized or fully human monoclonal antibody (i.e., an antibody derived from a single cell line). In a preferred embodiment, the CD27 agonist is varlilumab, or a fragment, derivative, conjugate, variant, or biosimilar thereof.

In a preferred embodiment, the CD27 agonist or CD27 binding molecule may also be a fusion protein. In a preferred embodiment, a multimeric CD27 agonist, such as a trimeric or hexameric CD27 agonist (with three or six ligand binding domains), may induce superior receptor (CD27L) clustering and internal cellular signaling complex formation compared to an agonistic monoclonal antibody, which typically possesses two ligand binding domains. Trimeric (trivalent) or hexameric (or hexavalent) or greater fusion proteins comprising three TNFRSF binding domains and IgG1-Fc and optionally further linking two or more of these fusion proteins are described, e.g., in Gieffers, et al., Mol. Cancer Therapeutics 2013, 12, 2735-47.

Agonistic CD27 antibodies and fusion proteins are known to induce strong immune responses. In a preferred embodiment, the CD27 agonist is a monoclonal antibody or fusion protein that binds specifically to CD27 antigen in a manner sufficient to reduce toxicity. In some embodiments, the CD27 agonist is an agonistic CD27 monoclonal antibody or fusion protein that abrogates antibody-dependent cellular toxicity (ADCC), for example NK cell cytotoxicity. In some embodiments, the CD27 agonist is an agonistic CD27 monoclonal antibody or fusion protein that abrogates antibody-dependent cell phagocytosis (ADCP). In some embodiments, the CD27 agonist is an agonistic CD27 monoclonal antibody or fusion protein that abrogates complement-dependent cytotoxicity (CDC). In some embodiments, the CD27 agonist is an agonistic CD27 monoclonal antibody or fusion protein which abrogates Fc region functionality.

In some embodiments, the CD27 agonists are characterized by binding to human CD27 (SEQ ID NO:127) with high affinity and agonistic activity. In an embodiment, the CD27 agonist is a binding molecule that binds to human CD27 (SEQ ID NO:127). In some embodiments, the CD27 agonists are characterized by binding to macaque CD27 (SEQ ID NO:128) with high affinity and agonistic activity. In an embodiment, the CD27 agonist is a binding molecule that binds to macaque CD27 (SEQ ID NO:128). The amino acid sequences of CD27 antigens to which a CD27 agonist or binding molecule binds is summarized in Table 16.

TABLE 16
Amino acid sequences of CD27 antigens.

| Identifier | Sequence (One-Letter Amino Acid Symbols) | |
|---|---|---|
| SEQ ID NO: 127 | MARPHPWWLC VLGTLVGLSA TPAPKSCPER HYWAQGKLCC QMCEPGTFLV KDCDQHRKAA | 60 |
| human CD27, | QCDPCIPGVS FSPDHHTRPH CESCRHCNSG LLVRNCTITA NAECACRNGW QCRDKECTEC | 120 |
| Tumor necrosis | DPLPNPSLTA RSSQALSPHP QPTHLPYVSE MLEARTAGHM QTLADFRQLP ARTLSTHWPP | 180 |
| factor receptor | QRSLCSSDFI RILVIFSGMF LVFTLAGALF LHQRRKYRSN KGESPVEPAE PCRYSCPREE | 240 |
| superfamily, | EGSTIPIQED YRKPEPACSP | 260 |
| member 7 (Homo | | |
| sapiens) | | |
| SEQ ID NO: 128 | MARPHPWWLC FLGTLVGLSA TPAPKSCPER HYWAQGKLCC QMCEPGTFLV KDCDQHRKAA | 60 |
| human CD27, | QCHPCIPGVS FSPDHHTRPH CESCRHCNSG LLIRNCTITA NAVCACRNGW QCRDKECTEC | 120 |
| Tumor necrosis | DPPPNPSLTT WPSQALGPHP QPTHLPYVNE MLEARTAGHM QTLADFRHLP ARTLSTHWPP | 180 |
| factor receptor | QRSLCSSDFI RILVIFSGMF LVFTLAGTLF LHQQRKYRSN KGESPMEPAE PCPYSCPREE | 240 |
| superfamily, | EGSTIPIQED YRKPEPASSP | 260 |
| member 7 (Macaca | | |
| nemestrina) | | |

In some embodiments, the compositions, processes and methods described include a CD27 agonist that binds human or murine CD27 with a K_{D} of about 100 pM or lower, binds human or murine CD27 with a K_{D} of about 90 pM or lower, binds human or murine CD27 with a K_{D} of about 80 pM or lower, binds human or murine CD27 with a K_{D} of about 70 pM or lower, binds human or murine CD27 with a K_{D} of about 60 pM or lower, binds human or murine CD27 with a K_{D} of about 50 pM or lower, binds human or murine CD27 with a K_{D} of about 40 pM or lower, or binds human or murine CD27 with a K_{D} of about 30 pM or lower.

In some embodiments, the compositions, processes and methods described include a CD27 agonist that binds to human or murine CD27 with a k_{assoc} of about 7.5×10⁵ l/M·s or faster, binds to human or murine CD27 with a k_{assoc} of about 7.5×10⁵ l/M·s or faster, binds to human or murine CD27 with a k_{assoc} of about 8×10⁵ l/M·s or faster, binds to human or murine CD27 with a k_{assoc} of about 8.5×10⁵ l/M·s or faster, binds to human or murine CD27 with a k_{assoc} of about 9×10⁵ l/M·s or faster, binds to human or murine CD27 with a k_{assoc} of about 9.5×10⁵ l/M·s or faster, or binds to human or murine CD27 with a k_{assoc} of about 1×10⁶ l/M·s or faster.

In some embodiments, the compositions, processes and methods described include a CD27 agonist that binds to human or murine CD27 with a k_{dissoc} of about 2×10⁻⁵ l/s or slower, binds to human or murine CD27 with a k_{dissoc} of about 2.1×10⁻⁵ l/s or slower, binds to human or murine CD27 with a k_{dissoc} of about 2.2×10⁻⁵ l/s or slower, binds to human or murine CD27 with a k_{dissoc} of about 2.3×10⁻⁵ l/s or slower, binds to human or murine CD27 with a k_{dissoc} of about 2.4×10⁻⁵ l/s or slower, binds to human or murine CD27 with a k_{dissoc} of about 2.5×10⁻⁵ l/s or slower, binds to human or murine CD27 with a k_{dissoc} of about 2.6×10⁻⁵ l/s or slower or binds to human or murine CD27 with a k_{dissoc} of about 2.7×10⁻⁵ l/s or slower, binds to human or murine CD27 with a k_{dissoc} of about 2.8×10⁻⁵ l/s or slower, binds to human or murine CD27 with a k_{dissoc} of about 2.9×10⁻⁵ l/s or slower, or binds to human or murine CD27 with a k_{dissoc} of about 3×10⁻⁵ l/s or slower.

In some embodiments, the compositions, processes and methods described include a CD27 agonist that binds to human or murine CD27 with an IC₅₀ of about 10 nM or lower, binds to human or murine CD27 with an IC₅₀ of about 9 nM or lower, binds to human or murine CD27 with an IC₅₀ of about 8 nM or lower, binds to human or murine CD27 with an IC₅₀ of about 7 nM or lower, binds to human or murine CD27 with an IC₅₀ of about 6 nM or lower, binds to human or murine CD27 with an IC₅₀ of about 5 nM or lower, binds to human or murine CD27 with an IC₅₀ of about 4 nM or lower, binds to human or murine CD27 with an IC₅₀ of about 3 nM or lower, binds to human or murine CD27 with an IC₅₀ of about 2 nM or lower, or binds to human or murine CD27 with an IC₅₀ of about 1 nM or lower.

In a preferred embodiment, the CD27 agonist is the monoclonal antibody varlilumab, also known as CDX-1127 or 1F5, or a fragment, derivative, variant, or biosimilar thereof. Varlilumab is available from Celldex Therapeutics, Inc. Varlilumab is an immunoglobulin G1-kappa, anti-[Homo sapiens anti-CD27 (TNFRSF7, tumor necrosis factor receptor superfamily member 7)], Homo sapiens monoclonal antibody. The amino acid sequences of varlilumab are set forth in Table 17. Varlilumab comprises N-glycosylation sites at positions 299 and 299″; heavy chain intrachain disulfide bridges at positions 22-96 (V_{H}-V_{L}), 146-202 (C_{H}1-C_{L}), 263-323 (C_{H}2) and 369-427 (C_{H}3) (and at positions 22″-96″, 146″-202″, 263″-323″, and 369″-427″); light chain intrachain disulfide bridges at positions 23′-88′ (V_{H}-V_{L}) and 134′-194′ (C_{H}1-C_{L}) (and at positions 23′″-88′″ and 134′″-194′″); interchain heavy chain-heavy chain disulfide bridges at positions 228-228″ and 231-231″; and interchain heavy chain-light chain disulfide bridges at 222-214′ and 222″-214′″. The preparation and properties of varlilumab are described in International Patent Application Publication No. WO 2016/145085 A2 and U.S. Patent Application Publication Nos. US 2011/0274685 A1 and US 2012/0213771 A1, the disclosures of which are incorporated by reference herein. Clinical and preclinical studies using varlilumab are known in the art and are described, for example, in Thomas, et al., OncoImmunology 2014, 3, e27255; Vitale, et al., Clin. Cancer Res. 2012, 18, 3812-21; and He, et al., J. Immunol. 2013, 191, 4174-83. Current clinical trials of varlilumab in a variety of hematological and solid tumor indications include U.S. National Institutes of Health clinicaltrials.gov identifiers NCT01460134, NCT02543645, NCT02413827, NCT02386111, and NCT02335918.

In an embodiment, a CD27 agonist comprises a heavy chain given by SEQ ID NO:129 and a light chain given by SEQ ID NO:130. In an embodiment, a CD27 agonist comprises heavy and light chains having the sequences shown in SEQ ID NO:129 and SEQ ID NO:130, respectively, or antigen binding fragments, Fab fragments, single-chain variable fragments (scFv), variants, or conjugates thereof. In an embodiment, a CD27 agonist comprises heavy and light chains that are each at least 99% identical to the sequences shown in SEQ ID NO:129 and SEQ ID NO:130, respectively. In an embodiment, a CD27 agonist comprises heavy and light chains that are each at least 98% identical to the sequences shown in SEQ ID NO:129 and SEQ ID NO:130, respectively. In an embodiment, a CD27 agonist comprises heavy and light chains that are each at least 97% identical to the sequences shown in SEQ ID NO:129 and SEQ ID NO:130, respectively. In an embodiment, a CD27 agonist comprises heavy and light chains that are each at least 96% identical to the sequences shown in SEQ ID NO:129 and SEQ ID NO:130, respectively. In an embodiment, a CD27 agonist comprises heavy and light chains that are each at least 95% identical to the sequences shown in SEQ ID NO:129 and SEQ ID NO:130, respectively.

In an embodiment, the CD27 agonist comprises the heavy and light chain CDRs or variable regions (VRs) of varlilumab. In an embodiment, the CD27 agonist heavy chain variable region (V_{H}) comprises the sequence shown in SEQ ID NO:131, and the CD27 agonist light chain variable region (V_{L}) comprises the sequence shown in SEQ ID NO:132, and conservative amino acid substitutions thereof. In an embodiment, a CD27 agonist comprises V_{H} and V_{L} regions that are each at least 99% identical to the sequences shown in SEQ ID NO:131 and SEQ ID NO:132, respectively. In an embodiment, a CD27 agonist comprises V_{H} and V_{L} regions that are each at least 98% identical to the sequences shown in SEQ ID NO:131 and SEQ ID NO:132, respectively. In an embodiment, a CD27 agonist comprises V_{H} and V_{L} regions that are each at least 97% identical to the sequences shown in SEQ ID NO:131 and SEQ ID NO:132, respectively. In an embodiment, a CD27 agonist comprises V_{H} and V_{L} regions that are each at least 96% identical to the sequences shown in SEQ ID NO:131 and SEQ ID NO:132, respectively. In an embodiment, a CD27 agonist comprises V_{H} and V_{L} regions that are each at least 95% identical to the sequences shown in SEQ ID NO:131 and SEQ ID NO:132, respectively.

In an embodiment, a CD27 agonist comprises heavy chain CDR1, CDR2 and CDR3 domains having the sequences set forth in SEQ ID NO:133, SEQ ID NO:134, and SEQ ID NO:135, respectively, and conservative amino acid substitutions thereof, and light chain CDR1, CDR2 and CDR3 domains having the sequences set forth in SEQ ID NO:136, SEQ ID NO:137, and SEQ ID NO:138, respectively, and conservative amino acid substitutions thereof.

In an embodiment, the CD27 agonist is a CD27 agonist biosimilar monoclonal antibody approved by drug regulatory authorities with reference to varlilumab. In an embodiment, the biosimilar monoclonal antibody comprises an CD27 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is varlilumab. In some embodiments, the one or more post-translational modifications are selected from one or more of: glycosylation, oxidation, deamidation, and truncation. In some embodiments, the biosimilar is a CD27 agonist antibody authorized or submitted for authorization, wherein the CD27 agonist antibody is provided in a formulation which differs from the formulations of a reference medicinal product or reference biological product, wherein the reference medicinal product or reference biological product is varlilumab. The CD27 agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is varlilumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is varlilumab.

TABLE 17
Amino acid sequences for CD27 agonist antibodies related to varlilumab.

| Identifier | Sequence (One-Letter Amino Acid Symbols) | |
|---|---|---|
| SEQ ID NO: 129 | QVQLVESGGG VVQPGRSLRL SCAASGFTFS SYDMHWVRQA PGKGLEWVAV IWYDGSNKYY | 60 |
| heavy chain for | ADSVKGRFTI SRDNSKNTLY LQMNSLRAED TAVYYCARGS GNWGFFDYWG QGTLVTVSSA | 120 |
| varlilumab | STKGPSVFPL APSSKSTSGG TAALGCLVKD YFPEPVTVSW NSGALTSGVH TFPAVLQSSG | 180 |
| | LYSLSSVVTV PSSSLGTQTY ICNVNHKPSN TKVDKKVEPK SCDKTHTCPP CPAPELLGGP | 240 |
| | SVFLFPPKPK DTLMISRTPE VTCVVVDVSH EDPEVKFNWY VDGVEVHNAK TKPREEQYNS | 300 |
| | TYRVVSVLTV LHQDWLNGKE YKCKVSNKAL PAPIEKTISK AKGQPREPQV YTLPPSRDEL | 360 |
| | TKNQVSLTCL VKGFYPSDIA VEWESNGQPE NNYKTTPPVL DSDGSFFLYS KLTVDKSRWQ | 420 |
| | QGNVFSCSVM HEALHNHYTQ KSLSLSPGKG SS | 452 |
| SEQ ID NO: 130 | DIQMTQSPSS LSASVGDRVT ITCRASQGIS RWLAWYQQKP EKAPKSLIYA ASSLQSGVPS | 60 |
| light chain for | RFSGSGSGTD FTLTISSLQP EDFATYYCQQ YNTYPRTFGQ GTKVEIKRTV AAPSVFIFPP | 120 |
| varlilumab | SDEQLKSGTA SVVCLLNNFY PREAKVQWKV DNALQSGNSQ ESVTEQDSKD STYSLSSTLT | 180 |
| | LSKADYEKHK VYACEVTHQG LSSPVTKSFN RGEC | 214 |
| SEQ ID NO: 131 | QVQLVESGGG VVQPGRSLRL SCAASGFTFS SYDMHWVRQA PGKGLEWVAV IWYDGSNKYY | 60 |
| heavy chain | ADSVKGRFTI SRDNSKNTLY LQMNSLRAED TAVYYCARGS GNWGFFDYWG QGTLVTVSS | 119 |
| variable region | | |
| for varlilumab | | |
| SEQ ID NO: 132 | DIQMTQSPSS LSASVGDRVT ITCRASQGIS RWLAWYQQKP EKAPKSLIYA ASSLQSGVPS | 60 |
| light chain | RFSGSGSGTD FTLTISSLQP EDFATYYCQQ YNTYPRTFGQ GTKVEIK | 107 |
| variable region | | |
| for varlilumab | | |
| SEQ ID NO: 133 | GFTFSSYD | 8 |
| heavy chain CDR1 | | |
| for varlilumab | | |
| SEQ ID NO: 134 | IWYDGSNK | 8 |
| heavy chain CDR2 | | |
| for varlilumab | | |
| SEQ ID NO: 135 | ARGSGNWGFF DY | 12 |
| heavy chain CDR3 | | |
| for varlilumab | | |
| SEQ ID NO: 136 | QGISRW | 6 |
| light chain CDR1 | | |
| for varlilumab | | |
| SEQ ID NO: 137 | AASG | 4 |
| light chain CDR2 | | |
| for varlilumab | | |
| SEQ ID NO: 138 | QQYNTYPRT | 9 |
| light chain CDR3 | | |
| for varlilumab | | |

In an embodiment, the CD27 agonist is an CD27 agonistic fusion protein as depicted in Structure I-A (C-terminal Fc-antibody fragment fusion protein) or Structure I-B (N-terminal Fc-antibody fragment fusion protein), or a fragment, derivative, conjugate, variant, or biosimilar thereof. The properties of structures I-A and I-B are described above and in U.S. Pat. Nos. 9,359,420, 9,340,599, 8,921,519, and 8,450,460, the disclosures of which are incorporated by reference herein. Amino acid sequences for the polypeptide domains of structure I-A are given in Table 6. The Fc domain preferably comprises a complete constant domain (amino acids 17-230 of SEQ ID NO:31) the complete hinge domain (amino acids 1-16 of SEQ ID NO:31) or a portion of the hinge domain (e.g., amino acids 4-16 of SEQ ID NO:31). Preferred linkers for connecting a C-terminal Fc-antibody may be selected from the embodiments given in SEQ ID NO:32 to SEQ ID NO:41, including linkers suitable for fusion of additional polypeptides. Likewise, amino acid sequences for the polypeptide domains of structure I-B are given in Table 7. If an Fc antibody fragment is fused to the N-terminus of an TNRFSF fusion protein as in structure I-B, the sequence of the Fc module is preferably that shown in SEQ ID NO:42, and the linker sequences are preferably selected from those embodiments set forth in SED ID NO:43 to SEQ ID NO:45.

In an embodiment, an CD27 agonist fusion protein according to structures I-A or I-B comprises one or more CD27 binding domains selected from the group consisting of a variable heavy chain and variable light chain of varlilumab, and fragments, derivatives, conjugates, variants, and biosimilars thereof.

In an embodiment, an CD27 agonist fusion protein according to structures I-A or I-B comprises one or more CD27 binding domains comprising an CD70 (CD27L) sequence (Table 18). In an embodiment, an CD27 agonist fusion protein according to structures I-A or I-B comprises one or more CD27 binding domains comprising a sequence according to SEQ ID NO:139. In an embodiment, an CD27 agonist fusion protein according to structures I-A or I-B comprises one or more CD27 binding domains comprising a soluble CD70 sequence. In an embodiment, a CD27 agonist fusion protein according to structures I-A or I-B comprises one or more CD27 binding domains comprising a sequence according to SEQ ID NO:140. In an embodiment, a CD27 agonist fusion protein according to structures I-A or I-B comprises one or more CD27 binding domains comprising a sequence according to SEQ ID NO:141.

In an embodiment, an CD27 agonist fusion protein according to structures I-A or I-B comprises one or more CD27 binding domains that is a scFv domain comprising V_{H} and V_{L} regions that are each at least 95% identical to the sequences shown in SEQ ID NO: 131 and SEQ ID NO: 132, respectively, wherein the V_{H} and V_{L} domains are connected by a linker.

TABLE 18
Additional polypeptide domains useful as CD27 binding domains in fusion proteins
(e.g., structures I-A and I-B).

| Identifier | Sequence (One-Letter Amino Acid Symbols) | |
|---|---|---|
| SEQ ID NO: 139 | MPEEGSGCSV RRRPYGCVLR AALVPLVAGL VICLVVCIQR FAQAQQQLPL ESLGWDVAEL | 60 |
| CD70 (CD27L) | QLNHTGPQQD PRLYWQGGPA LGRSFLHGPE LDKGQLRIHR DGIYMVHIQV TLAICSSTTA | 120 |
| | SRHHPTTLAV GICSPASRSI SLLRLSFHQG CTIASQRLTP LARGDTLCTN LTGTLLPSRN | 180 |
| | TDETFFGVQW VRP | 193 |
| SEQ ID NO: 140 | SLGWDVAELQ LNHTGPQQDP RLYWQGGPAL GRSFLHGPEL DKGQLRIHRD GIYMVHIQVT | 60 |
| CD70 soluble | LAICSSTTAS RHHPTTLAVG ICSPASRSIS LLRLSFHQGC TIASQRLTPL ARGDTLCTNL | 120 |
| domain | TGTLLPSRNT DETFFGVQWV RP | 142 |
| SEQ ID NO: 141 | VAELQLNHTG PQQDPRLYWQ GGPALGRSFL HGPELDKGQL RIHRDGIYMV HIQVTLAICS | 60 |
| CD70 soluble | STTASRHHPT TLAVGICSPA SRSISLLRLS FHQGCTIASQ RLTPLARGDT LCTNLTGTLL | 120 |
| domain | PSRNTDETFF GVQWVRP | 137 |
| (alternative) | | |

In an embodiment, the CD27 agonist is a CD27 agonistic single-chain fusion polypeptide comprising (i) a first soluble CD27 binding domain, (ii) a first peptide linker, (iii) a second soluble CD27 binding domain, (iv) a second peptide linker, and (v) a third soluble CD27 binding domain, further comprising an additional domain at the N-terminal and/or C-terminal end, and wherein the additional domain is a Fab or Fc fragment domain. In an embodiment, the CD27 agonist is a CD27 agonistic single-chain fusion polypeptide comprising (i) a first soluble CD27 binding domain, (ii) a first peptide linker, (iii) a second soluble CD27 binding domain, (iv) a second peptide linker, and (v) a third soluble CD27 binding domain, further comprising an additional domain at the N-terminal and/or C-terminal end, wherein the additional domain is a Fab or Fc fragment domain wherein each of the soluble CD27 binding domains lacks a stalk region (which contributes to trimerisation and provides a certain distance to the cell membrane, but is not part of the CD27 binding domain) and the first and the second peptide linkers independently have a length of 3-8 amino acids.

In an embodiment, the CD27 agonist is an CD27 agonistic single-chain fusion polypeptide comprising (i) a first soluble tumor necrosis factor (TNF) superfamily cytokine domain, (ii) a first peptide linker, (iii) a second soluble TNF superfamily cytokine domain, (iv) a second peptide linker, and (v) a third soluble TNF superfamily cytokine domain, wherein each of the soluble TNF superfamily cytokine domains lacks a stalk region and the first and the second peptide linkers independently have a length of 3-8 amino acids, and wherein the TNF superfamily cytokine domain is an CD27 binding domain.

In an embodiment, the CD27 agonist is a CD27 agonist described in U.S. Patent Application Publication No. US 2014/0112942 A1, US 2011/0274685 A1, or US 2012/0213771 A1, or International Patent Application Publication No. WO 2012/004367 A1, the disclosures of which are incorporated by reference herein.

In an embodiment, the CD27 agonist is a CD27 agonistic scFv antibody comprising any of the foregoing V_{H} domains linked to any of the foregoing V_{L} domains.

### GITR (CD357) Agonists

In an embodiment, the TNFRSF agonist is a GITR agonist. The GITR agonist may be any GITR binding molecule known in the art. The GITR binding molecule may be a monoclonal antibody or fusion protein capable of binding to human or mammalian GITR. The GITR agonists or GITR binding molecules may comprise an immunoglobulin heavy chain of any isotype (e.g., IgG, IgE, IgM, IgD, IgA, and IgY), class (e.g., IgG1, IgG2, IgG3, IgG4, IgA1 and IgA2) or subclass of immunoglobulin molecule. The GITR agonist or GITR binding molecule may have both a heavy and a light chain. As used herein, the term binding molecule also includes antibodies (including full length antibodies), monoclonal antibodies (including full length monoclonal antibodies), polyclonal antibodies, multispecific antibodies (e.g., bispecific antibodies), human, humanized or chimeric antibodies, and antibody fragments, e.g., Fab fragments, F(ab′) fragments, fragments produced by a Fab expression library, epitope-binding fragments of any of the above, and engineered forms of antibodies, e.g., scFv molecules, that bind to OX40. In an embodiment, the GITR agonist is an antigen binding protein that is a fully human antibody. In an embodiment, the GITR agonist is an antigen binding protein that is a humanized antibody. In some embodiments, GITR agonists for use in the presently disclosed methods and compositions include anti-GITR antibodies, human anti-GITR antibodies, mouse anti-OX40 antibodies, mammalian anti-GITR antibodies, monoclonal anti-OX40 antibodies, polyclonal anti-OX40 antibodies, chimeric anti-OX40 antibodies, anti-OX40 adnectins, anti-OX40 domain antibodies, single chain anti-OX40 fragments, heavy chain anti-OX40 fragments, light chain anti-OX40 fragments, anti-OX40 fusion proteins, and fragments, derivatives, conjugates, variants, or biosimilars thereof. In a preferred embodiment, the OX40 agonist is an agonistic, anti-OX40 humanized or fully human monoclonal antibody (i.e., an antibody derived from a single cell line).

In a preferred embodiment, the GITR agonist or GITR binding molecule may also be a fusion protein. In a preferred embodiment, a multimeric GITR agonist, such as a trimeric or hexameric GITR agonist (with three or six ligand binding domains), may induce superior GITR receptor clustering and internal cellular signaling complex formation compared to an agonistic monoclonal antibody, which typically possesses two ligand binding domains. Trimeric (trivalent) or hexameric (or hexavalent) or greater fusion proteins comprising three TNFRSF binding domains and IgG1-Fc and optionally further linking two or more of these fusion proteins are described, e.g., in Gieffers, et al., Mol. Cancer Therapeutics 2013, 12, 2735-47.

In some embodiments, the anti-GITR antibodies are characterized by binding to hGITR (SEQ ID NO:142) with high affinity, in the presence of a stimulating agent, e.g., CD3 antibody (muromonab or OKT3), and are agonistic, and abrogate the suppression of T effector cells by Treg cells. In an embodiment, the GITR binding molecule binds to human GITR (SEQ ID NO:142). In an embodiment, the GITR binding molecule binds to murine GITR (SEQ ID NO:143). The amino acid sequences of GITR antigens to which a GITR binding molecule binds are summarized in Table 19.

TABLE 19
Amino acid sequences of GITR antigens.

| Identifier | Sequence (One-Letter Amino Acid Symbols) | |
|---|---|---|
| SEQ ID NO: 142 | MAQHGAMGAF RALCGLALLC ALSLGQRPTG GPGCGPGRLL LGTGTDARCC RVHTTRCCRD | 60 |
| human GITR, | YPGEECCSEW DCMCVQPEFH CGDPCCTTCR HHPCPPGQGV QSQGKFSFGF QCIDCASGTF | 120 |
| tumor necrosis | SGGHEGHCKP WTDCTQFGFL TVFPGNKTHN AVCVPGSPPA EPLGWLTVVL LAVAACVLLL | 160 |
| factor receptor | TSAQLGLHIW QLRSQCMWPR ETQLLLEVPP STEDARSCQF PEEERGERSA EEKGRLGDLW | 240 |
| superfamily | V | 241 |
| member 18 (Homo | | |
| sapiens) | | |
| SEQ ID NO: 143 | MGAWAMLYGV SMLCVLDLGQ PSVVEEPGCG PGKVQNGSGN NTRCCSLYAP GKEDCPKERC | 60 |
| murine GITR, | ICVTPEYHCG DPQCKICKHY PCQPGQRVES QGDIVFGFRC VACAMGTFSA GRDGHCRLWT | 120 |
| tumor necrosis | NCSQFGFLTM FPGNKTHNAV CIPEPLPTEQ YGHLTVIFLV MAACIFFLTT VQLGLHIWQL | 180 |
| factor receptor | RRQHMCPRET QPFAEVQLSA EDACSFQFPE EERGEQTEEK CHLGGRWP | 228 |
| superfamily | | |
| member 18 (Mus | | |
| musculus) | | |

In an embodiment, the GITR agonist is an antigen binding protein that is a fully human antibody. In an embodiment, the GITR agonist is an antigen binding protein that is a humanized antibody. In an embodiment, the GITR agonist is an antigen binding protein that agonizes the activity of human GITR. In an embodiment, the GITR binding molecule is an antigen binding protein that is a fully human IgG1 antibody. In an embodiment, the GITR agonist is an antigen binding protein that is capable of binding Fcgamma receptor (FcγR). In an embodiment, the GITR agonist is an antigen binding protein that is capable of binding Fcgamma receptor (FcγR) such that a cluster of antigen binding proteins is formed.

In some embodiments, the compositions, processes and methods described include a GITR agonist that binds human or murine GITR with a K_{D} of about 100 pM or lower, binds human or murine GITR with a K_{D} of about 90 pM or lower, binds human or murine GITR with a K_{D} of about 80 pM or lower, binds human or murine GITR with a K_{D} of about 70 pM or lower, binds human or murine GITR with a K_{D} of about 60 pM or lower, binds human or murine GITR with a K_{D} of about 50 pM or lower, binds human or murine GITR with a K_{D} of about 40 pM or lower, or binds human or murine GITR with a K_{D} of about 30 pM or lower.

In some embodiments, the compositions, processes and methods described include a GITR agonist that binds to human or murine GITR with a k_{assoc} of about 7.5×10⁵ l/M·s or faster, binds to human or murine GITR with a k_{assoc} of about 7.5×10⁵ l/M·s or faster, binds to human or murine GITR with a k_{assoc} of about 8×10⁵ l/M·s or faster, binds to human or murine GITR with a k_{assoc} of about 8.5×10⁵ l/M·s or faster, binds to human or murine GITR with a k_{assoc} of about 9×10⁵ l/M·s or faster, binds to human or murine GITR with a k_{assoc} of about 9.5×10⁵ l/M·s or faster, or binds to human or murine GITR with a k_{assoc} of about 1×10⁶ l/M·s or faster.

In some embodiments, the compositions, processes and methods described include a GITR agonist that binds to human or murine GITR with a k_{dissoc} of about 2×10⁻⁵ l/s or slower, binds to human or murine GITR with a k_{dissoc} of about 2.1×10⁻⁵ l/s or slower, binds to human or murine GITR with a k_{dissoc} of about 2.2×10⁻⁵ l/s or slower, binds to human or murine GITR with a k_{dissoc} of about 2.3×10⁻⁵ l/s or slower, binds to human or murine GITR with a k_{dissoc} of about 2.4×10⁻⁵ l/s or slower, binds to human or murine GITR with a k_{dissoc} of about 2.5×10⁻⁵ l/s or slower, binds to human or murine GITR with a k_{dissoc} of about 2.6×10⁻⁵ l/s or slower or binds to human or murine GITR with a k_{dissoc} of about 2.7×10⁻⁵ l/s or slower, binds to human or murine GITR with a k_{dissoc} of about 2.8×10⁻⁵ l/s or slower, binds to human or murine GITR with a k_{dissoc} of about 2.9×10⁻⁵ l/s or slower, or binds to human or murine GITR with a k_{dissoc} of about 3×10⁻⁵ l/s or slower.

In some embodiments, the compositions, processes and methods described include a GITR agonist that binds to human or murine GITR with an IC₅₀ of about 10 nM or lower, binds to human or murine GITR with an IC₅₀ of about 9 nM or lower, binds to human or murine GITR with an IC₅₀ of about 8 nM or lower, binds to human or murine GITR with an IC₅₀ of about 7 nM or lower, binds to human or murine GITR with an IC₅₀ of about 6 nM or lower, binds to human or murine GITR with an IC₅₀ of about 5 nM or lower, binds to human or murine GITR with an IC₅₀ of about 4 nM or lower, binds to human or murine GITR with an IC₅₀ of about 3 nM or lower, binds to human or murine GITR with an IC₅₀ of about 2 nM or lower, or binds to human or murine GITR with an IC₅₀ of about 1 nM or lower.

In a preferred embodiment, the GITR agonist is an agonistic, anti-GITR monoclonal antibody (i.e., an antibody derived from a single cell line). Agonist anti-GITR antibodies are known to induce strong immune responses. Cohen, et al., Cancer Res. 2006, 66, 4904-12; Schaer, et al., Curr. Opin. Investig. Drugs 2010, 11, 1378-1386. In a preferred embodiment, the GITR agonist is a monoclonal antibody that binds specifically to GITR antigen. In an embodiment, the GITR agonist is a GITR receptor blocker. In some embodiments, the GITR agonist is an agonistic, anti-GITR monoclonal antibody that abrogates antibody-dependent cellular toxicity (ADCC), for example NK cell cytotoxicity. In some embodiments, the GITR agonist is an agonistic, anti-GITR monoclonal antibody that abrogates antibody-dependent cell phagocytosis (ADCP). In some embodiments, the GITR agonist is an agonistic, anti-GITR monoclonal antibody that abrogates complement-dependent cytotoxicity (CDC).

In an embodiment, the GITR agonist is the agonistic, anti-GITR monoclonal antibody TRX518 (TolerRx, Inc.), also known as 6C8 and Ch-6C8-Agly. TRX518 is a fully-humanized IgG1 anti-human GITR monoclonal antibody in which heavy chain asparagine 297 is substituted with alanine to eliminate N-linked glycosylation, which abrogates Fc region functionality, including ADCC and CDC. Rosenzweig, et al., J. Clin. Oncol. 2010, 28 (supplement; abstract e13028); Jung, et al., Cur. Opin. Biotechnology 2011, 22, 858-867. The amino acid sequences of TRX518 are set forth in Table 20. In some embodiments, the GITR binding molecule is the anti-human-GITR monoclonal antibody 6C8, or a variant thereof. The 6C8 antibody is an anti-GITR antibody that binds to human GITR on immune cells, e.g., human T cells and dendritic cells, with high affinity. Preferably, such binding molecules abrogate the suppression of T effector cells by Treg cells and are agonistic to partially activated immune cells in vitro in the presence of a stimulating agent, such as CD3. In some embodiments, the GITR binding molecule is the anti-murine GITR monoclonal antibody 2F8, or a variant thereof. The preparation, properties, and uses of 6C8 and 2F8 antibodies, and their variants, are described in U.S. Pat. Nos. 7,812,135; 8,388,967; and 9,028,823; the disclosures of which are incorporated by reference herein.

In an embodiment, the agonistic anti-GITR monoclonal antibody comprises a heavy chain selected from the group consisting of SEQ ID NO:144, SEQ ID NO:145, SEQ ID NO:146, and SEQ ID NO:147, and a light chain comprising SEQ ID NO:148. In an embodiment, the agonistic anti-GITR monoclonal antibody comprises a heavy chain with a sequence identity of greater than 99% to a sequence selected from the group consisting of SEQ ID NO:144, SEQ ID NO:145, SEQ ID NO:146, and SEQ ID NO:147, and a light chain with a sequence identity of greater than 99% to SEQ ID NO:148. In an embodiment, the agonistic anti-GITR monoclonal antibody comprises a heavy chain with a sequence identity of greater than 98% to a sequence selected from the group consisting of SEQ ID NO:144, SEQ ID NO:145, SEQ ID NO:146, and SEQ ID NO:147, and a light chain with a sequence identity of greater than 98% to SEQ ID NO:148. In an embodiment, the agonistic anti-GITR monoclonal antibody comprises a heavy chain with a sequence identity of greater than 95% to a sequence selected from the group consisting of SEQ ID NO:144, SEQ ID NO:145, SEQ ID NO:146, and SEQ ID NO:147, and a light chain with a sequence identity of greater than 95% to SEQ ID NO:148. In an embodiment, the agonistic anti-GITR monoclonal antibody comprises a heavy chain with a sequence identity of greater than 90% to a sequence selected from the group consisting of SEQ ID NO:144, SEQ ID NO:145, SEQ ID NO:146, and SEQ ID NO:147, and a light chain with a sequence identity of greater than 90% to SEQ ID NO:148.

In an embodiment, the agonistic anti-GITR monoclonal antibody comprises a heavy chain that comprises the leader sequence of SEQ ID NO:149 and further comprises a sequence selected from the group consisting of SEQ ID NO:144, SEQ ID NO:145, SEQ ID NO:146 and SEQ ID NO:147. In an embodiment, the agonistic anti-GITR monoclonal antibody comprises a light chain that comprises the leader sequence of SEQ ID NO:148 and further comprises a sequence comprising SEQ ID NO:150.

In an embodiment, the agonistic anti-GITR monoclonal antibody (such as TRX518) comprises a variable heavy chain region (V_{H}) selected from the group consisting of SEQ ID NO:151 and SEQ ID NO:152, and a variable light chain region (V_{L}) comprising SEQ ID NO:153. In an embodiment, the agonistic anti-GITR monoclonal antibody comprises a variable heavy chain region selected from the group consisting of amino acid residues 20-138 of SEQ ID NO:151 and amino acid residues 20-138 of SEQ ID NO:152, and a variable light chain region comprising amino acid residues 20-138 of SEQ ID NO:153. In an embodiment, the agonistic anti-GITR monoclonal antibody comprises a variable heavy chain region with a sequence identity of greater than 99% to a sequence selected from the group consisting of amino acid residues 20-138 of SEQ ID NO:151 and amino acid residues 20-138 of SEQ ID NO:152, and a variable light chain region with a sequence identity of greater than 99% to a sequence comprising amino acid residues 20-138 of SEQ ID NO:153. In an embodiment, the agonistic anti-GITR monoclonal antibody comprises a variable heavy chain region with a sequence identity of greater than 98% to a sequence selected from the group consisting of amino acid residues 20-138 of SEQ ID NO:151 and amino acid residues 20-138 of SEQ ID NO:152, and a variable light chain region with a sequence identity of greater than 98% to a sequence comprising amino acid residues 20-138 of SEQ ID NO:153. In an embodiment, the agonistic anti-GITR monoclonal antibody comprises a variable heavy chain region with a sequence identity of greater than 95% to a sequence selected from the group consisting of amino acid residues 20-138 of SEQ ID NO:151 and amino acid residues 20-138 of SEQ ID NO:152, and a variable light chain region with a sequence identity of greater than 95% to a sequence comprising amino acid residues 20-138 of SEQ ID NO:153. In an embodiment, the agonistic anti-GITR monoclonal antibody comprises a variable heavy chain region with a sequence identity of greater than 90% to a sequence selected from the group consisting of amino acid residues 20-138 of SEQ ID NO:151 and amino acid residues 20-138 of SEQ ID NO:152, and a variable light chain region with a sequence identity of greater than 90% to a sequence comprising amino acid residues 20-138 of SEQ ID NO:153.

In an embodiment, the agonistic anti-GITR monoclonal antibody comprises a V_{H} region comprising at least one CDR1 region comprising the amino acid sequence of SEQ ID NO:154; at least one CDR2 region comprising an amino acid sequence selected from the group consisting of SEQ ID NO:155 and SEQ ID NO:156; and at least one CDR3 region comprising the amino acid sequence of SEQ ID NO:157; and a V_{L} region comprising at least one CDR1 region comprising the amino acid sequence of SEQ ID NO: 158; at least one CDR2 region comprising the amino acid sequence of SEQ ID NO:159; and at least one CDR3 region comprising the amino acid sequence of SEQ ID NO:160. In an embodiment, the invention provides isolated nucleic acid molecules encoding a polypeptide sequence comprising a 6C8 CDR, e. g., comprising an amino acid sequence selected from the group consisting of: SEQ ID NO:154, SEQ ID NO:155, SEQ ID NO:156, SEQ ID NO:157, SEQ ID NO:158, SEQ ID NO:159, and SEQ ID NO:160. In an embodiment, the agonistic anti-GITR monoclonal antibody comprises the six CDRs represented by the amino acid sequences of SEQ ID NO:154, SEQ ID NO:156, SEQ ID NO:157, SEQ ID NO:158, SEQ ID NO:159, and SEQ ID NO:160. In an embodiment, the GITR binding molecule that specifically binds to GITR comprises the six CDRs represented by the amino acid sequences of SEQ ID NO:154, SEQ ID NO:155, SEQ ID NO:157, SEQ ID NO:158, SEQ ID NO:159, and SEQ ID NO:160. In an embodiment, the agonistic anti-GITR monoclonal antibody comprises a V_{L} having at least one CDR domain comprising an amino acid sequence selected from the group consisting of SEQ ID NO:158, SEQ ID NO:159, and SEQ ID NO:160. In an embodiment, the agonistic anti-GITR monoclonal antibody comprises a V_{L} having at least two CDR domains comprising an amino acid sequence selected from the group consisting of SEQ ID NO:158, SEQ ID NO:159, and SEQ ID NO:160. In an embodiment, the agonistic anti-GITR monoclonal antibody comprises a V_{L} having CDR domains comprising the amino acid sequences of SEQ ID NO:158, SEQ ID NO:159, and SEQ ID NO:160. In an embodiment, the agonistic anti-GITR monoclonal antibody comprises a V_{L} having at least one CDR domain comprising an amino acid sequence selected from the group consisting of SEQ ID NO:154, SEQ ID NO:155, and SEQ ID NO:157. In an embodiment, the agonistic anti-GITR monoclonal antibody comprises a V_{L} having at least two CDR domains comprising an amino acid sequence selected from the group consisting of SEQ ID NO:154, SEQ ID NO:155, and SEQ ID NO:157. In an embodiment, the agonistic anti-GITR monoclonal antibody comprises a V_{L} having CDR domains comprising the amino acid sequences of SEQ ID NO:154, SEQ ID NO:155, and SEQ ID NO:157. In an embodiment, the agonistic anti-GITR monoclonal antibody comprises a V_{L} having at least one CDR domain comprising an amino acid sequence selected from the group consisting of SEQ ID NO:154, SEQ ID NO:156, and SEQ ID NO:157. In an embodiment, the agonistic anti-GITR monoclonal antibody comprises a V_{L} having at least two CDR domains comprising an amino acid sequence selected from the group consisting of SEQ ID NO:154, SEQ ID NO:156, and SEQ ID NO:157. In an embodiment, the agonistic anti-GITR monoclonal antibody comprises a V_{L} having CDR domains comprising the amino acid sequences of SEQ ID NO:154, SEQ ID NO:156, and SEQ ID NO:157. In an embodiment, the agonistic anti-GITR monoclonal antibody comprises a V_{H} domain comprising a CDR set forth in SEQ ID NO:154 (CDR1). In an embodiment, the agonistic anti-GITR monoclonal antibody comprises a V_{H} domain comprising a CDR set forth in SEQ ID NO:155 (CDR2, “N” variant). In an embodiment, the agonistic anti-GITR monoclonal antibody comprises a V_{H} domain comprising a CDR set forth in SEQ ID NO:156 (CDR3, “Q” variant). In an embodiment, the agonistic anti-GITR monoclonal antibody comprises a V_{H} domain comprising a CDR set forth in SEQ ID NO:157 (CDR3). In an embodiment, the agonistic anti-GITR monoclonal antibody comprises a V_{L} domain comprising a CDR set forth in SEQ ID NO:158 (CDR1). In an embodiment, the agonistic anti-GITR monoclonal antibody comprises a V_{L} domain comprising a CDR set forth in SEQ ID NO:159 (CDR2). In an embodiment, the agonistic anti-GITR monoclonal antibody comprises a V_{L} domain comprising a CDR set forth in SEQ ID NO:160 (CDR3).

In an embodiment, the agonistic anti-GITR monoclonal antibody is a chimeric 6C8 monoclonal antibody, or an antigen-binding fragment, derivative, conjugate, or variant thereof. In an embodiment, the agonistic anti-GITR monoclonal antibody comprises a heavy chain selected from the group consisting of SEQ ID NO:162 and SEQ ID NO:163, and a light chain comprising SEQ ID NO:161. In an embodiment, the agonistic anti-GITR monoclonal antibody comprises a heavy chain with a sequence identity of greater than 99% to a sequence selected from the group consisting of SEQ ID NO:162 and SEQ ID NO:163, and a light chain with a sequence identity of greater than 99% to SEQ ID NO:161. In an embodiment, the agonistic anti-GITR monoclonal antibody comprises a heavy chain with a sequence identity of greater than 98% to a sequence selected from the group consisting of SEQ ID NO:162 and SEQ ID NO:163, and a light chain with a sequence identity of greater than 98% to SEQ ID NO:161. In an embodiment, the agonistic anti-GITR monoclonal antibody comprises a heavy chain with a sequence identity of greater than 95% to a sequence selected from the group consisting of SEQ ID NO:162 and SEQ ID NO:163, and a light chain with a sequence identity of greater than 95% to SEQ ID NO: 161. In an embodiment, the agonistic anti-GITR monoclonal antibody comprises a heavy chain with a sequence identity of greater than 90% to a sequence selected from the group consisting of SEQ ID NO:162 and SEQ ID NO:163, and a light chain with a sequence identity of greater than 90% to SEQ ID NO:161.

In an embodiment, the GITR agonist is a GITR agonist biosimilar monoclonal antibody approved by drug regulatory authorities with reference to TRX518 or 6C8. In an embodiment, the biosimilar monoclonal antibody comprises an GITR antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is TRX518 or 6C8. In some embodiments, the one or more post-translational modifications are selected from one or more of: glycosylation, oxidation, deamidation, and truncation. In some embodiments, the biosimilar is a GITR agonist antibody authorized or submitted for authorization, wherein the GITR agonist antibody is provided in a formulation which differs from the formulations of a reference medicinal product or reference biological product, wherein the reference medicinal product or reference biological product is TRX518 or 6C8. The GITR agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is TRX518 or 6C8. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is TRX518 or 6C8.

TABLE 20
Amino acid sequences for GITR agonist antibodies related to TRX518 and 6C8.

| Identifier | Sequence (One-Letter Amino Acid Symbols) | |
|---|---|---|
| SEQ ID NO: 144 | QVTLRESGPA LVKPTQTLTL TCTFSGFSLS TSGMGVGWIR QPPGKALEWL AHIWWDDDKY | 60 |
| humanized 6C8 | YNPSLKSRLT ISKDTSKNQV VLTMTNMDPV DTATYYCART RRYFPFAYWG QGTLVTVSSA | 120 |
| heavy chain | STKGPSVFPL APSSKSTSGG TAALGCLVKD YFPEPVTVSW NSGALTSGVH TFPAVLQSSG | 180 |
| variant | LYSLSSVVTV PSSSLGTQTY ICNVNHKPSN TKVDKKVEPK SCDKTHTCPP CPAPELLGGP | 240 |
| | SVFLFPPKPK DTLMISRTPE VTCVVVDVSH EDPEVKFNWY VDGVEVHNAK TKPREEQYNS | 300 |
| | TYRVVSVLTV LHQDWLNGKE YKCKVSNKAL PAPIEKTISK AKGQPREPQV YTLPPSRDEL | 360 |
| | TKNQVSLTCL VKGFYPSDIA VEWESNGQPE NNYKTTPPVL DSDGSFFLYS KLTVDKSRWQ | 420 |
| | QGNVFSCSVM HEALHNHYTQ KSLSLSPGK | 449 |
| SEQ ID NO: 145 | QVTLRESGPA LVKPTQTLTL TCTFSGFSLS TSGMGVGWIR QPPGKALEWL AHIWWDDDKY | 60 |
| humanized 6C8 | YNPSLKSRLT ISKDTSKNQV VLTMTNMDPV DTATYYCART RRYFPFAYWG QGTLVTVSSA | 120 |
| heavy chain | STKGPSVFPL APSSKSTSGG TAALGCLVKD YFPEPVTVSW NSGALTSGVH TFPAVLQSSG | 180 |
| variant | LYSLSSVVTV PSSSLGTQTY ICNVNHKPSN TKVDKKVEPK SCDKTHTCPP CPAPELLGGP | 240 |
| | SVFLFPPKPK DTLMISRTPE VTCVVVDVSH EDPEVKFNWY VDGVEVHNAK TKPREEQYAS | 300 |
| | TYRVVSVLTV LHQDWLNGKE YKCKVSNKAL PAPIEKTISK AKGQPREPQV YTLPPSRDEL | 360 |
| | TKNQVSLTCL VKGFYPSDIA VEWESNGQPE NNYKTTPPVL DSDGSFFLYS KLTVDKSRWQ | 420 |
| | QGNVFSCSVM HEALHNHYTQ KSLSLSPGK | 449 |
| SEQ ID NO: 146 | QVTLRESGPA LVKPTQTLTL TCTFSGFSLS TSGMGVGWIR QPPGKALEWL AHIWWDDDKY | 60 |
| humanized 6C8 | YQPSLKSRLT ISKDTSKNQV VLTMTNMDPV DTATYYCART RRYFPFAYWG QGTLVTVSSA | 120 |
| heavy chain | STKGPSVFPL APSSKSTSGG TAALGCLVKD YFPEPVTVSW NSGALTSGVH TFPAVLQSSG | 160 |
| variant | LYSLSSVVTV PSSSLGTQTY ICNVNHKPSN TKVDKKVEPK SCDKTHTCPP CPAPELLGGP | 240 |
| | SVFLFPPKPK DTLMISRTPE VTCVVVDVSH EDPEVKFNWY VDGVEVHNAK TKPREEQYNS | 300 |
| | TYRVVSVLTV LHQDWLNGKE YKCKVSNKAL PAPIEKTISK AKGQPREPQV YTLPPSRDEL | 360 |
| | TKNQVSLTCL VKGFYPSDIA VEWESNGQPE NNYKTTPPVL DSDGSFFLYS KLTVDKSRWQ | 420 |
| | QGNVFSCSVM HEALHNHYTQ KSLSLSPGK | 449 |
| SEQ ID NO: 147 | QVTLRESGPA LVKPTQTLTL TCTFSGFSLS TSGMGVGWIR QPPGKALEWL AHIWWDDDKY | 60 |
| humanized 6C8 | YQPSLKSRLT ISKDTSKNQV VLTMTNMDPV DTATYYCART RRYFPFAYWG QGTLVTVSSA | 120 |
| heavy chain | STKGPSVFPL APSSKSTSGG TAALGCLVKD YFPEPVTVSW NSGALTSGVH TFPAVLQSSG | 180 |
| variant | LYSLSSVVTV PSSSLGTQTY ICNVNHKPSN TKVDKKVEPK SCDKTHTCPP CPAPELLGGP | 240 |
| | SVFLFPPKPK DTLMISRTPE VTCVVVDVSH EDPEVKFNWY VDGVEVHNAK TKPREEQYAS | 300 |
| | TYRVVSVLTV LHQDWLNGKE YKCKVSNKAL PAPIEKTISK AKGQPREPQV YTLPPSRDEL | 360 |
| | TKNQVSLTCL VKGFYPSDIA VEWESNGQPE NNYKTTPPVL DSDGSFFLYS KLTVDKSRWQ | 420 |
| | QGNVFSCSVM HEALHNHYTQ KSLSLSPGK | 449 |
| SEQ ID NO: 148 | EIVMTQSPAT LSVSPGERAT LSCKASQNVG TNVAWYQQKP GQAPRLLIYS ASYRYSGIPA | 60 |
| humanized 6C8 | RFSGSGSGTE FTLTISSLQS EDFAVYYCQQ YNTDPLTFGG GTKVEIKRTV AAPSVFIFPP | 120 |
| light chain | SDEQLKSGTA SVVCLLNNFY PREAKVQWKV DNALQSGNSQ ESVTEQDSKD STYSLSSTLT | 180 |
| | LSKADYEKHK VYACEVTHQG LSSPVTKSFN RGEC | 214 |
| SEQ ID NO: 149 | MDRLTFSFLL LIVPAYVLS | 19 |
| 6C8 heavy chain | | |
| leader | | |
| SEQ ID NO: 150 | METQSQVFVY MLLWLSGVDG | 20 |
| 6C8 light chain | | |
| leader | | |
| SEQ ID NO: 151 | MDRLTFSFLL LIVPAYVLSQ VTLKESGPGI LKPSQTLSLT CSFSGFSLST SGMGVGWIRQ | 60 |
| humanized 6C8 | PSGKGLEWLA HIWWDDDKYY NPSLKSQLTI SKDTSRNQVF LKITSVDTAD AATYYCARTR | 120 |
| heavy chain | RYFPFAYWGQ GTLVTVSS | 138 |
| variable region | | |
| variant | | |
| SEQ ID NO: 152 | MDRLTFSFLL LIVPAYVLSQ VTLKESGPGI LKPSQTLSLT CSFSGFSLST SGMGVGWIRQ | 60 |
| humanized 6C8 | PSGKGLEWLA HIWWDDDKYY QPSLKSQLTI SKDTSRNQVF LKITSVDTAD AATYYCARTR | 120 |
| heavy chain | RYFPFAYWGQ GTLVTVSS | 138 |
| variable region | | |
| variant | | |
| SEQ ID NO: 153 | METQSQVFVY MLLWLSGVDG DIVMTQSQKF MSTSVGDRVS VTCKASQNVG TNVAWYQQKP | 60 |
| humanized 6C8 | GQSPKALIYS ASYRYSGVPD RFTGSGSGTD FTLTINNVHS EDLAEYFCQQ YNTDPLTFGA | 120 |
| light chain | GTKLEIK | 127 |
| variable region | | |
| SEQ ID NO: 154 | GFSLSTSGMG VG | 12 |
| 6C8 heavy chain | | |
| CDR1 | | |
| SEQ ID NO: 155 | HIWWDDDKYY NPSLKS | 16 |
| 6C8 heavy chain | | |
| CDR2 variant | | |
| SEQ ID NO: 156 | HIWWDDDKYY QPSLKS | 16 |
| 6C8 heavy chain | | |
| CDR2 variant | | |
| SEQ ID NO: 157 | TRRYFPFAY | 9 |
| 6C8 heavy chain | | |
| CDR3 | | |
| SEQ ID NO: 158 | KASQNVGTNV A | 11 |
| 6C8 light chain | | |
| CDR1 | | |
| SEQ ID NO: 159 | SASYRYS | 7 |
| 6C8 light chain | | |
| CDR2 | | |
| SEQ ID NO: 160 | QQYNTDPLT | 9 |
| 6C8 light chain | | |
| CDR3 | | |
| SEQ ID NO: 161 | QVTLKESGPG ILKPSQTLSL TCSFSGFSLS TSGMGVGWIR QPSGKGLEWL AHIWWDDDKY | 60 |
| chimeric 6C8 | YNPSLKSQLT ISKDTSRNQV FLKITSVDTA DAATYYCART RRYFPFAYWG QGTLVTVSSA | 120 |
| heavy chain | STKGPSVFPL APSSKSTSGG TAALGCLVKD YFPEPVTVSW NSGALTSGVH TFPAVLQSSG | 180 |
| variant | LYSLSSVVTV PSSSLGTQTY ICNVNHKPSN TKVDKKVEPK SCDKTHTCPP CPAPELLGGP | 240 |
| | SVFLFPPKPK DTLMISRTPE VTCVVVDVSH EDPEVKFNWY VDGVEVHNAK TKPREEQYNS | 300 |
| | TYRVVSVLTV LHQDWLNGKE YKCKVSNKAL PAPIEKTISK AKGQPREPQV YTLPPSRDEL | 360 |
| | TKNQVSLTCL VKGFYPSDIA VEWESNGQPE NNYKTTPPVL DSDGSFFLYS KLTVDKSRWQ | 420 |
| | QGNVFSCSVM HEALHNHYTQ KSLSLSPGK | 449 |
| SEQ ID NO: 162 | QVTLKESGPG ILKPSQTLSL TCSFSGFSLS TSGMGVGWIR QPSGKGLEWL AHIWWDDDKY | 60 |
| chimeric 6C8 | YNPSLKSQLT ISKDTSRNQV FLKITSVDTA DAATYYCART RRYFPFAYWG QGTLVTVSSA | 120 |
| heavy chain | STKGPSVFPL APSSKSTSGG TAALGCLVKD YFPEPVTVSW NSGALTSGVH TFPAVLQSSG | 180 |
| variant | LYSLSSVVTV PSSSLGTQTY ICNVNHKPSN TKVDKKVEPK SCDKTHTCPP CPAPELLGGP | 240 |
| | SVFLFPPKPK DTLMISRTPE VTCVVVDVSH EDPEVKFNWY VDGVEVHNAK TKPREEQYAS | 300 |
| | TYRVVSVLTV LHQDWLNGKE YKCKVSNKAL PAPIEKTISK AKGQPREPQV YTLPPSRDEL | 360 |
| | TKNQVSLTCL VKGFYPSDIA VEWESNGQPE NNYKTTPPVL DSDGSFFLYS KLTVDKSRWQ | 420 |
| | QGNVFSCSVM HEALHNHYTQ KSLSLSPGK | 449 |
| SEQ ID NO: 163 | DIVMTQSQKF MSTSVGDRVS VTCKASQNVG TNVAWYQQKP GQSPKALIYS ASYRYSGVPD | 60 |
| chimeric 6C8 | RFTGSGSGTD FTLTINNVHS EDLAEYFCQQ YNTDPLTFGA GTKLEIKRTV AAPSVFIFPP | 120 |
| light chain | SDEQLKSGTA SVVCLLNNFY PREAKVQWKV DNALQSGNSQ ESVTEQDSKD STYSLSSTLT | 180 |
| variant | LSKADYEKHK VYACEVTHQG LSSPVTKSFN RGEC | 214 |

In an embodiment, the GITR agonist is an agonistic anti-GITR monoclonal antibody with described in U.S. Pat. No. 8,709,424; U.S. Patent Application Publication Nos. US 2012/0189639 A1 and US 2014/0348841 A1, and International Patent Application Publication No. WO 2011/028683 A1 (Merck Sharp & Dohme Corp.), the disclosures of which are incorporated by reference herein. In an embodiment, the GITR agonist is an agonistic, anti-GITR monoclonal antibody selected from the group consisting of 36E5, 3D6, 61G6, 6116, 61F6, 1D8, 17F10, 35D8, 49A1, 9E5, and 31H6, and fragments, variants, derivatives, or biosimilars thereof. The structure, properties, and preparation of these antibodies are described in U.S. Pat. No. 8,709,424; U.S. Patent Application Publication Nos. US 2012/0189639 A1 and US 2014/0348841 A1, the disclosures of which are incorporated herein by reference.

In some embodiments, the agonistic, anti-GITR monoclonal antibody comprises a humanized heavy chain variable domain (V_{H}) comprising a sequence selected from the group consisting of SEQ ID NO:164, SEQ ID NO:166, SEQ ID NO:168, SEQ ID NO:170, SEQ ID NO:172, SEQ ID NO: 174, SEQ ID NO:176, SEQ ID NO:178, SEQ ID NO:180, SEQ ID NO: 182, SEQ ID NO: 184, SEQ ID NO:186, SEQ ID NO:188, SEQ ID NO:190, SEQ ID NO: 192, SEQ ID NO: 194, SEQ ID NO:196, SEQ ID NO:198, SEQ ID NO:200, SEQ ID NO:202, SEQ ID NO:204, SEQ ID NO:206, or a variant, fragment, or biosimilar thereof, and a humanized heavy chain variable domain (V_{H}) comprising a sequence selected from the group consisting of SEQ ID NO:165, SEQ ID NO:167, SEQ ID NO:169, SEQ ID NO:171, SEQ ID NO:173, SEQ ID NO:175, SEQ ID NO:177, SEQ ID NO:179, SEQ ID NO:181, SEQ ID NO:183, SEQ ID NO:185, SEQ ID NO:187, SEQ ID NO:189, SEQ ID NO:191, SEQ ID NO:193, SEQ ID NO:195, SEQ ID NO:197, SEQ ID NO:199, SEQ ID NO:201, SEQ ID NO:203, SEQ ID NO:205, SEQ ID NO:207, or a variant, fragment, or biosimilar thereof (Table 21). In some embodiments, the agonistic, anti-GITR monoclonal antibody further comprises a heavy chain constant region, wherein the heavy chain constant region comprises a γ1, γ2, γ3, or 74 human heavy chain constant region or a variant thereof. In some embodiments, the light chain constant region comprises a lambda or a kappa human light chain constant region.

In an embodiment, the GITR agonist is a GITR agonist biosimilar monoclonal antibody approved by drug regulatory authorities with reference to 36E5, 3D6, 61G6, 6H6, 61F6, 1D8, 17F10, 35D8, 49A1, 9E5, and 31H6. In an embodiment, the biosimilar monoclonal antibody comprises an GITR antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 36E5, 3D6, 61G6, 6H6, 61F6, 1D8, 17F10, 35D8, 49A1, 9E5, and 31H6. In some embodiments, the one or more post-translational modifications are selected from one or more of: glycosylation, oxidation, deamidation, and truncation. In some embodiments, the biosimilar is a GITR agonist antibody authorized or submitted for authorization, wherein the GITR agonist antibody is provided in a formulation which differs from the formulations of a reference medicinal product or reference biological product, wherein the reference medicinal product or reference biological product is 36E5, 3D6, 61G6, 6H6, 61F6, 1D8, 17F10, 35D8, 49A1, 9E5, and 31H6. The GITR agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 36E5, 3D6, 61G6, 6H6, 61F6, 1D8, 17F10, 35D8, 49A1, 9E5, and 31H6. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 36E5, 3D6, 61G6, 6116, 61F6, 1D8, 17F10, 35D8, 49A1, 9E5, and 31H6.

TABLE 21
Amino acid sequences for GITR agonist antibodies related to the GITR agonists
described in International Patent Application Publication No. WO 2011/028683 A1.

| Identifier | Sequence (One-Letter Amino Acid Symbols) | |
|---|---|---|
| SEQ ID NO: 164 | EVNLVESGGG LVKPGGSLKV SCAASGFTFS SYAMSWVRQT PEKRLEWVAS ISSGGTTYYP | 60 |
| 36E5 heavy chain | DSVKGRFTIS RDNARNILYL QMSSLRSEDT AMYYCARVGG YYDSMDYWGQ GISVTDSS | 118 |
| variable region | | |
| SEQ ID NO: 165 | DIVLTQSPAS LAVSLGQRAT ISCRASESVD NYGVSFMNWF QQKPGQPPKL LIYAASNQGS | 60 |
| 36E5 light chain | GVPARFSGSG SGTDFSLNIH PMEEDDTAMY FCQQTKEVTW TFGGGTKLEI KRA | 113 |
| variable region | | |
| SEQ ID NO: 166 | EVQLVESGGG LVQPGRSLKL SCAASGFTFS DYYMAWVRQA PTKGLEWVAY IHANGGSTYY | 60 |
| 3D6 heavy chain | RDSVRGRFSI SRDNGKSTLY LQMDSLRSED TATYYCTTGS FMYAADYYIM DAWGQGASVT | 120 |
| variable region | VSS | 123 |
| SEQ ID NO: 167 | DVVMTQTPVS LSVSLGNQAS ISCRSSQSLL HSDGNTFLSW YFQKPGQSPQ LLIYLASNRF | 60 |
| 3D6 light chain | SGVSNRFSGS GSGTDFTLKI SRVEPEDLGV YYCFQHTHLP LTFGSGTKLE IKR | 113 |
| variable region | | |
| SEQ ID NO: 168 | DVQLQESGPG LVKPSQSLSL TCTVTGYSIT SDYAWNWIRQ FPGNKLEWMG YISYSGSTRY | 60 |
| 61G6 heavy chain | NPSLKSRISI TRDTSKNQFF LQLNSVTSED TATYYCARQL GLRFFDYWGQ GTTLTVSS | 118 |
| variable region | | |
| SEQ ID NO: 169 | QIVLTQSPAL MSASPGEKVT MTCSANSTVN YMYWYQQKPR SSPKPCIYLT SNLASGVPAR | 60 |
| 61G6 light chain | FSGSGSGTSY SLTISSMEAE DAATYYCQQW NSNPPTFGAG TKLELRRA | 108 |
| variable region | | |
| SEQ ID NO: 170 | QVQLQQSGAE LMKPGASVKI SCKATGYTFS RYWIEWIKQR PGHGLEWIGE ILPGSGSSNY | 60 |
| 6H6 heavy chain | NEKFKDKATF TADTSSNTAY MQFSSLTSED SAVYYCARKV YYYAMDFWGQ GTSVTVSS | 118 |
| variable region | | |
| SEQ ID NO: 171 | QIVLTQSPAI MSVSLGERVT VTCTASSSVS SSYFHWYQQK PGSSPKLWIY STSNLASGVP | 60 |
| 6H6 light chain | ARFSGSGSGT SYSLTISTME AEDAATYYCH QYHRSPRTFG GGTKLEIKRA | 110 |
| variable region | | |
| SEQ ID NO: 172 | QVQLQQSGAE LARPGASVKM SCKASGYTFT SYTMHWVKQR PGQGLEWIGY INPRSVYTNY | 60 |
| 61F6 heavy chain | NQKFKDKATL TADKSSSTAY MQLSSLTSED SAVYYCARLG GYYDTMDYWG QGTSVTVSS | 119 |
| variable region | | |
| SEQ ID NO: 173 | DIVVTQSPAS LAVSLGQRAT ISCRASESVD NYGISFMNWF QQKPGQPPKL LIYAASNQGS | 60 |
| 61F6 light chain | GVPARFSGSG SGTDFSLNIH PMEEDDTAVY FCQQSKEVPF TFGSGTKLEI KRA | 113 |
| variable region | | |
| SEQ ID NO: 174 | QVTLKESGPG ILKPSQTLSL TCSFSGFSLS TSGMGVGWIR QPSGKGLEWL AHIWWDDDKY | 60 |
| 1D8 heavy chain | YSPSLKSQLT ISKDTSRNQV FLKITSLDTA DTATYYCVRS YYYGSSGAMD YWGQGTSVTV | 120 |
| variable region | SS | 122 |
| SEQ ID NO: 175 | DIVMTQTPLS LPVSLGDQAS ISCRSSQSLV HSDGNTYLHW YLQKPGQSPK LLIYKVSKRF | 60 |
| 1D8 light chain | SGVPDRFSGS GSGTDFTLKI SRVEAEDLGV YFCSQSTHVP PTFGGGTKLE IKRADAAP | 118 |
| variable region | | |
| SEQ ID NO: 176 | EVKLVESGGG FVKPGGSLKL SCAASGFTVR NYAMSWVRQT PEKRLEWVAS ISTGDRSYLP | 60 |
| 17F10 heavy | DSMKGRFTIS RDNARNILYL QMSSLRSEDT AIYYCQRYFD FDSFAFWGQG TLVTVSA | 117 |
| chain variable | | |
| region | | |
| SEQ ID NO: 177 | DIQMTQTPSS LSASLGDRVT ISCRASQDIN NFLNWYQQKP DGSLKLLIYY TSKLHSGVPS | 60 |
| 17F10 light | RFSGSGSGTD FSLTISNLDQ EDVATYFCQQ GHTLPPTFGG GTKLEVKRAD AAP | 113 |
| chain variable | | |
| region | | |
| SEQ ID NO: 178 | EVQLQESGPS LVKPSQTLSL TCSVTGDSIT SGYWNWIRKF PGNKLEYMGY ISYSGSTYYN | 60 |
| 35D8 heavy chain | PSLRGRISIT RDTSKSQYYL QLSSVTTEDT ATYYCSRRHL GSGYGWFAYW GQGTLVTVSA | 120 |
| variable region | | |
| SEQ ID NO: 179 | DIVMTQSHKF MSTSVGDRVS ITCKASQDVN TAVAWYQQKP GQSPKLLIYW ASTRHTGVPD | 60 |
| 35D8 light chain | RFTGSGSGTD YALTINSVQA EDLALYYCQQ HSYTPPWTFG GGTKLEIRRA DAAP | 114 |
| variable region | | |
| SEQ ID NO: 180 | EVQLQESGPS LVKPSQTLSL TCSVTGDSIT SGYWNWIRKF PGNKFEYMGF ISYSGNTYYN | 60 |
| 49A1 heavy chain | PSLRSRISIT RDTSKNQYFL HLNSVTTEDT ATYYCSRRHL ISGYGWFAYW GQGTLVTVSA | 120 |
| variable region | | |
| SEQ ID NO: 181 | VIVMTQSHKF MSTSIGDRVN ITCKASQDVI SAVAWYQQKP GQSPKLLIYW ASTRHTGVPD | 60 |
| 49A1 light chain | RFTGSGSGTD FTLTINSVQA EDRALYYCQQ HSYTPPWTFG GGTNLEIKRA DAAP | 114 |
| variable region | | |
| SEQ ID NO: 182 | QVTLKESGPG ILQPSQTLSL TCTFSGFSLS TYGVGVGWIR QPSGKGLEWL ANIWWDDDNY | 60 |
| 9E5 heavy chain | YNPSLIHRLT VSKDTSNNQA FLKITNVDTA ETATYYCAQI KEPRDWFFEF WGPGTMVSVS | 120 |
| variable region | S | 121 |
| SEQ ID NO: 183 | DIQMTQTPSS MPASLGERVT IFCRASQGVN NFLTWYQQKP DGTIKPLIFY TSNLQSGVPS | 60 |
| 9E5 light chain | RFSGSGSGTD YSLSISSLEP EDFAMYYCQQ YHGFPNTFGA GTKLELKRAD AAP | 113 |
| variable region | | |
| SEQ ID NO: 184 | QVTLKESGPG ILQPSQTLSL TCTFSGFSLS TYGVGVGWIR QPSGKGLEWL ANIWWDDDKY | 60 |
| 31H6 heavy chain | YNPSLKNRLT ISKDTSNNQA FLKITNVDTA ETATYYCAQI KEPRDWFFEF WGPGTMVSVS | 120 |
| variable region | S | 121 |
| SEQ ID NO: 185 | DIQMTQTPSS MPASLGERVT IFCRASQGVN NYLTWYQQKP DGTIKPLIFY TSNLQSGVPS | 60 |
| 31H6 light chain | RFSGSGSGTD YSLSISSLEP EDFAMYYCQQ YHGFPNTFGA GTKLELKRAD AAP | 113 |
| variable region | | |
| SEQ ID NO: 186 | QVQLVESGGG VVQPGRSLRL SCAASGFTFS SYAMSWVRQA PGKGLEWVAS ISSGGTTYYP | 60 |
| humanized 36E5 | DSVKGRFTIS RDNSKNTLYL QMNSLRAEDT AVYYCARVGG YYDSMDYWGQ GTLVTVSS | 118 |
| heavy chain | | |
| variable region | | |
| SEQ ID NO: 187 | EIVLTQSPGT LSLSPGERAT LSCRASESVD XYGVSFMNWY QQKPGQAPRL LIYAASXQGS | 60 |
| humanized 36E5 | GIPDRFSGSG SGTDFTLTIS RLEPEDFAVY YCQQTKEVTW TFGQGTKVEI KR | 112 |
| light chain | | |
| variable region | | |
| SEQ ID NO: 188 | QVQLVESGGG VVQPGRSLRL SCAASGFTFS DYYMAWVRQA PGKGLEWVAY IHANGGSTYY | 60 |
| humanized 3D6 | RDSVRGRFTI SRDNSKNTLY LQMNSLRAED TAVYYCXXGS FMYAADYYIM DAWGQGTLVT | 120 |
| heavy chain | VSS | 123 |
| variable region | | |
| SEQ ID NO: 189 | DIVMTQSPLS LPVTPGEPAS ISCRSSQSLL HSDGNTFLSW YLQKPGQSPQ LLIYLASNRF | 60 |
| humanized 3D6 | SGVPDRFSGS GSGTDFTLKI SRVEAEDVGV YYCFQHTHLP LTFGQGTKVE IKR | 113 |
| light chain | | |
| variable region | | |
| SEQ ID NO: 190 | QVQLQESGPG LVKPSETLSL TCTVSGYSIT SDYAWNWIRQ PPGKGLEWXG YISYSGSTRY | 60 |
| humanized 61G6 | NPSLKSRXTI SXDTSKNQFS LKLSSVTAAD TAVYYCARQL GLRFFDYWGQ GTLVTVSS | 118 |
| heavy chain | | |
| variable region | | |
| SEQ ID NO: 191 | EIVLTQSPGT LSLSPGERAT LSCSANSTVN YMYWYQQKPG QAPRXXIYLT SNLASGIPDR | 60 |
| humanized 61G6 | FSGSGSGTDF TLTISRLEPE DFAVYYCQQW NSNPPTFGQG TKVEIKR | 107 |
| light chain | | |
| variable region | | |
| SEQ ID NO: 192 | QVQLVQSGAE VKKPGASVKV SCKASGYTFS RYWIEWVRQA PGQGLEWXGE ILPGSGSSNY | 60 |
| humanized 6H6 | NEKFKDRXTX TXDTSTSTAY MELRSLRSDD TAVYYCARKV YYYAMDFWGQ GTLVTVSS | 118 |
| heavy chain | | |
| variable region | | |
| SEQ ID NO: 193 | EIVLTQSPGT LSLSPGERAT LSCTASSSVS SSYFHWYQQK PGQAPRLXIY STSNLASGIP | 60 |
| humanized 6H6 | DRFSGSGSGT DXTLTISRLE PEDFAVYYCH QYHRSPRTFG QGTKVEIKR | 109 |
| light chain | | |
| variable region | | |
| SEQ ID NO: 194 | QVQLVQSGAE VKKPGASVKV SCKASGYTFT SYTMHWVRQA PGQGLEWXGY INPRSVYTNY | 60 |
| humanized 61F6 | NQKFKDRXTX TXDXSTSTAY MELRSLRSDD TAVYYCARLG GYYDTMDYWG QGTLVTVSS | 119 |
| heavy chain | | |
| variable region | | |
| SEQ ID NO: 195 | DIQMTQSPSS LSASVGDRVT ITCRASESVD NYGISFMNWY QQKPGKAPKL LIYAASNQGS | 60 |
| humanized 61F6 | GVPSRFSGSG SGTDFTLTIS SLQPEDFATY YCQQSKEVPF TFGQGTKVEI KR | 112 |
| light chain | | |
| variable region | | |
| SEQ ID NO: 196 | QVQLVESGGG VVQPGRSLRL SCAXSGFSLS TSGMGVGWVR QAPGKGLEWV AHIWWDDDKY | 60 |
| humanized 1D8 | YSPSLKSRXT ISXDXSKNTX YLQMNSLRAE DTAVYYCXRS YYYGSSGAMD YWGQGTLVTV | 120 |
| heavy chain | SS | 122 |
| variable region | | |
| SEQ ID NO: 197 | DIVMTQSPLS LPVTPGEPAS ISCRSSQSLV HSDGNTYLHW YLQKPGQSPQ LLIYKVSKRF | 60 |
| humanized 1D8 | SGVPDRFSGS GSGTDFTLKI SRVEAEDVGV YYCSQSTHVP PTFGQGTKVE IKR | 113 |
| light chain | | |
| variable region | | |
| SEQ ID NO: 198 | QVQLVESGGG VVQPGRSLRL SCAASGFTVR NYAMSWVRQA PGKGLEWVAS ISTGDRSYLP | 60 |
| humanized 17F10 | DSMKGRFTIS RDNSKNTLYL QMNSLRAEDT AVYYCXRYFD FDSFAFWGQG TLVTVSS | 117 |
| heavy chain | | |
| variable region | | |
| SEQ ID NO: 199 | DIQMTQSPSS LSASVGDRVT ITCRASQDIN NFLNWYQQKP GKAPKLLIYY TSKLHSGVPS | 60 |
| humanized 17F10 | RFSGSGSGTD FTLTISSLQP EDFATYYCQQ GHTLPPTFGQ GTKVEIKR | 108 |
| light chain | | |
| variable region | | |
| SEQ ID NO: 200 | QVQLQESGPG LVKPSETLSL TCTVSGDSIT SGYWNWIRQP PGKGLEXXGY ISYSGSTYYN | 60 |
| humanized 35D8 | PSLRGRVTIS XDTSKNQFSL KLSSVTAADT AVYYCXRRHL GSGYGWFAYW GQGTLVTVSS | 120 |
| heavy chain | | |
| variable region | | |
| SEQ ID NO: 201 | DIVMTQSPDS LAVSLGERAT INCKASQDVN TAVAWYQQKP GQPPKLLIYW ASTRHTGVPD | 60 |
| humanized 35D8 | RFSGSGSGTD XTLTISSLQA EDVAVYYCQQ HSYTPPWTFG QGTKVEIKR | 109 |
| light chain | | |
| variable region | | |
| SEQ ID NO: 202 | QVQLQESGPG LVKPSETLSL TCTVSGDSIT SGYWNWIRQP PGKGLEXXGF ISYSGNTYYN | 60 |
| humanized 49A1 | PSLRSRXTIS XDTSKNQXSL KLSSVTAADT AVYYCXRRHL ISGYGWFAYW GQGTLVTVSS | 120 |
| heavy chain | | |
| variable region | | |
| SEQ ID NO: 203 | XIVMTQSPDS LAVSLGERAT INCKASQDVI SAVAWYQQKP GQPPKLLIYW ASTRHTGVPD | 60 |
| humanized 49A1 | RFSGSGSGTD FTLTISSLQA EDVAVYYCQQ HSYTPPWTFG QGTKVEIKR | 109 |
| light chain | | |
| variable region | | |
| SEQ ID NO: 204 | QVQLQESGPG LVKPSETLSL TCTXSGFSLS TYGVGVGWIR QPPGKGLEWX XNIWWDDDNY | 60 |
| humanized 9E5 | YNPSLIHRXT XSXDTSKNQX SLKLSSVTAA DTAVYYCAXI KEPRDWFFEF WGQGTLVTVS | 120 |
| heavy chain | S | 121 |
| variable region | | |
| SEQ ID NO: 205 | DIQMTQSPSS LSASVGDRVT ITCRASQGVN NFLTWYQQKP GKAPKXLIXY TSNLQSGVPS | 60 |
| humanized 9E5 | RFSGSGSGTD XTLTISSLQP EDFATYYCQQ YHGFPNTFGQ GTKVEIKR | 108 |
| light chain | | |
| variable region | | |
| SEQ ID NO: 206 | QVQLQESGPG LVKPSETLSL TCTXSGFSLS TYGVGVGWIR QPPGKGLEWX XNIWWDDDKY | 60 |
| humanized 31H6 | YNPSLKNRXT ISXDTSKNQX SLKLSSVTAA DTAVYYCAXI KEPRDWFFEF WGQGTLVTVS | 120 |
| heavy chain | S | 121 |
| variable region | | |
| SEQ ID NO: 207 | DIQMTQSPSS LSASVGDRVT ITCRASQGVN NYLTWYQQKP GKAPKXLIXY TSNLQSGVPS | 60 |
| humanized 31H6 | RFSGSGSGTD XTLTISSLQP EDFATYYCQQ YHGFPNTFGQ GTKVEIKR | 108 |
| light chain | | |
| variable region | | |

In an embodiment, the GITR agonist is an agonistic, anti-GITR monoclonal antibody described in U.S. Patent Application Publication No. US 2013/0108641 A1 (Sanofi SA) and International Patent Application Publication No. WO 2011/028683 A1 (Sanofi SA), the disclosures of which are incorporated by reference herein. In an embodiment, a GITR binding molecule includes monoclonal antibodies and variants and fragments thereof, including humanized and chimeric recombinant antibodies, that bind human GITR, comprising a heavy chain variable domain (V_{H}) selected from the group consisting of SEQ ID NO:208, SEQ ID NO:210, SEQ ID NO: 211, SEQ ID NO: 212, SEQ ID NO: 213, SEQ ID NO: 214, SEQ ID NO:219, SEQ ID NO:221, SEQ ID NO:223, and SEQ ID NO:225, and a light chain variable domain (V_{L}) selected from the group consisting of SEQ ID NO:209, SEQ ID NO:215, SEQ ID NO:216, SEQ ID NO:217, SEQ ID NO:218, SEQ ID NO:220, SEQ ID NO:222, SEQ ID NO:224, and SEQ ID NO:226 (Table 22). In an embodiment, the GITR binding molecule is an agonistic, anti-GITR monoclonal antibody comprising (a) one, two, or three heavy chain CDRs selected from the group consisting of SEQ ID NO:227, SEQ ID NO:228, SEQ ID NO:229, SEQ ID NO:233, SEQ ID NO:234, SEQ ID NO:235, SEQ ID NO:240, SEQ ID NO:241, SEQ ID NO:242, SEQ ID NO:243, SEQ ID NO:244, SEQ ID NO:245, SEQ ID NO:249, and conservative amino acid substitutions thereof, and (b) one, two, or three light chain CDRs selected from the group consisting of SEQ ID NO:230, SEQ ID NO:231, SEQ ID NO:232, SEQ ID NO:236, SEQ ID NO:237, SEQ ID NO:238, SEQ ID NO:239, SEQ ID NO:246, SEQ ID NO:247, SEQ ID NO:248, and conservative amino acid substitutions thereof (Table 22). In an embodiment, the GITR agonist is an agonistic, anti-GITR monoclonal antibody selected from the group consisting of 2155, 698, 706, 827, 1649, and 1718, and fragments, derivatives, variants, biosimilars, and combinations thereof.

In an embodiment, the GITR agonist is a GITR agonist biosimilar monoclonal antibody approved by drug regulatory authorities with reference to 2155, 698, 706, 827, 1649, and 1718. In an embodiment, the biosimilar monoclonal antibody comprises an GITR antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 2155, 698, 706, 827, 1649, and 1718. In some embodiments, the one or more post-translational modifications are selected from one or more of: glycosylation, oxidation, deamidation, and truncation. In some embodiments, the biosimilar is a GITR agonist antibody authorized or submitted for authorization, wherein the GITR agonist antibody is provided in a formulation which differs from the formulations of a reference medicinal product or reference biological product, wherein the reference medicinal product or reference biological product is 2155, 698, 706, 827, 1649, and 1718. The GITR agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 2155, 698, 706, 827, 1649, and 1718. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 2155, 698, 706, 827, 1649, and 1718.

TABLE 22
Amino acid sequences for GITR agonist antibodies related to the GITR agonists
described in International Patent Application Publication No. WO 2011/028683 A1.

| Identifier | Sequence (One-Letter Amino Acid Symbols) | |
|---|---|---|
| SEQ ID NO: 208 | EVKLVESGGG LVKPGGSLKL SCGASGFTIS SYAMSWVRQS PEKRLEWVAI ISTGGSTYYP | 60 |
| 2155 variable | DSVRGRFTIS RDNARNSLYL QMSSLRSEDT AMYYCARVGG YYDSMDHWGQ GTSVTVSS | 118 |
| heavy chain | | |
| SEQ ID NO: 209 | DIVLTQSPAS LAVSLGQRAT ISCRASETVD NYGISFMNWF QQKPGQSPKL LIYAASNQGS | 60 |
| 2155 variable | GVPARFSGSG SGTDFSLNIH PMEEDDTAMY FCQQSKEVPW TFGGGTKLEI K | 111 |
| light chain | | |
| SEQ ID NO: 210 | QVTLVESGGG LVKPGGSLTL SCGASGFTIS SYAMSWVRQS PGKALEWVAI ISTGGSTYYP | 60 |
| 2155 humanized | DSVRGRFTIS RDNAKNSLYL TMSSLDSVDT AMYYCARVGG YYDSMDHWGQ GTSVT | 115 |
| (HC1) heavy | | |
| chain | | |
| SEQ ID NO: 211 | QVTLVESGGG LVKPGGSLTL SCGASGFTIS SYAMSWVRQS PGKALEWVAI ISTGGSTYYP | 60 |
| 2155 humanized | DSVRGRFTIS RDNAKNSLYL TMSSLDSVDT ATYYCARVGG YYDSMDHWGQ GTSVT | 115 |
| (HC2) heavy | | |
| chain | | |
| SEQ ID NO: 212 | QVTLVESGGG LVKPGGSLTL SCGASGFTIS SYAMSWVRQS PGKALEWVAI ISTGGSTYYP | 60 |
| 2155 humanized | DKFRGRFTIS RDNAKNSLYL TMSSLRSEDT ATYYCARVGG YYDSMDHWGQ GTSVT | 115 |
| (HC3a) heavy | | |
| chain | | |
| SEQ ID NO: 213 | QVTLKESGGG LVKPGGSLTL SCGASGFTIS SYAMSWVRQS PGKALEWVAI ISTGGSTYYP | 60 |
| humanized (HC3b) | DKFRGRFTIS RDNAKNSLYL TMSSLRSEDT ATYYCARVGG YYDSMDHWGQ GTSVT | 115 |
| heavy chain | | |
| SEQ ID NO: 214 | EVQLVESGGG LIQPGGSLKL SCAASGFTIS SYAMSWVRQA PGKGLEWVAI ISTGGSTYYA | 60 |
| humanized (HC4) | DSVKGRFTIS RDNSKNTLYL QMNSLRAEDT AVYYCARVGG YYDSMDHWGQ GTSVT | 115 |
| heavy chain | | |
| SEQ ID NO: 215 | DIVLTQSPAS LAASVGDRAT ISCRASETVD NYGISFMNWF QQKPGKSPKL LIYAASNQGS | 60 |
| 2155 humanized | GVPARFSGSG SGTDFSLNIH PMQPDDTATY FCQQSKEVPW TFGGGTKLE | 109 |
| (LC1) light | | |
| chain | | |
| SEQ ID NO: 216 | DIVLTQSPAS LSASVGDRAT ISCRASETVD NYGISFMNWF QQKPGQSPKL LIYAASNQGS | 60 |
| 2155 humanized | GVPARFSGSG SGTDFSLTIS PMQPDDTATY YCQQSKEVPW TFGGGTKLE | 109 |
| (LC2a) light | | |
| chain | | |
| SEQ ID NO: 217 | DIVLTQSPAS LSASVGDRAT ISCRASETVD NYGISYMNWF QQKPGQSPKL LIYAASNQGS | 60 |
| 2155 humanized | GVPARFSGSG SGTDFSLTIS PMQPDDTATY YCQQSKEVPW TFGGGTKLE | 109 |
| (LC2b) light | | |
| chain | | |
| SEQ ID NO: 218 | DIVLTQSPAS LAVSPGQRAT ITCRASETVD NYGISFMNWF QQKPGQPPKL LIYAASNQGS | 60 |
| 2155 humanized | GVPARFSGSG SGTDFTLTIN PVEADDTANY YCQQSKEVPW TFGQGTKVE | 109 |
| (LC3) light | | |
| chain | | |
| SEQ ID NO: 219 | EVQLQQSGTV LARPGASVKM SCEASGYSFT TYWMHWIKQR PGQGLEWIGA IYPGNSDTGY | 60 |
| 698 variable | NQKFKGKAKL TAVTSATTAY MELSSLTDED SAVYYCTRTS TYPHFDYWGQ GTTLTVSS | 118 |
| heavy chain | | |
| SEQ ID NO: 220 | DILLTQSPAI LSVSPGERVS FSCRASQSIG TSIHWYQQRT NGSPRLLIKY ASESISGIPS | 60 |
| 698 variable | RFSGSGSGTD FTLNINSVES EDIADYYCQQ SNNWPLTFGA GTKLELK | 107 |
| light chain | | |
| SEQ ID NO: 221 | EVQLQQSGTV LARPGASVKM SCEASGYSFT TYWMHWIKQR PGQGLEWIGA IYPGNSDTGY | 60 |
| 706 variable | NQKFKGKAKL TAVTSASTAY MELSSLTNED SAVYYCTRTS TYPHFDYWGQ GTTLTVSS | 118 |
| heavy chain | | |
| SEQ ID NO: 222 | DILLTQSPAI LSVSPGERVS FSCRASQSIG TSIHWYQQRT NGSPRLLIKY ASESISGIPS | 60 |
| 706 variable | RFSGSGSGTD FTLNINSVES EDIADYYCQQ TNNWPLTFGA GTKLELK | 107 |
| light chain | | |
| SEQ ID NO: 223 | EVQLQQSGTV LARPGASVKM SCETSGYSFT TYWIHWIKQR PGQGLEWIAT IYPGNSDAGY | 60 |
| 827 variable | NQKFRGKAKL TAVTSASTAY MELSSLTNED SAVYYCTRSS TYPHFDYWGQ GTTLTVSS | 118 |
| heavy chain | | |
| SEQ ID NO: 224 | DILLTQSPAI LSVSPGERVS FSCRASQSIG TSIHWYQQRT NDSPRLLIKY ASESISGIPS | 60 |
| 827 variable | RFSGSGSGTD FTLNINSVES EDIADYYCQQ TNNWPLTFGA GTKLELK | 107 |
| light chain | | |
| SEQ ID NO: 225 | QVQVQQSGPE LVKPGASVRI SCKASDYTFT NYYIHWVRQR PGQGLEWLGW IYPGKGYTNY | 60 |
| 1718 variable | NEKFKGKATL TADKSSSTAY MQFSSLTSED SAVYFCASGY GNYYFPYWGQ GTLVTVSA | 118 |
| heavy chain | | |
| SEQ ID NO: 226 | IQMTQSSSYL SVSLGGRVTI TCKASDHIKN WLAWYQQKPG NVPRLLMSAA TSLETGFPSR | 60 |
| 1718 variable | FSGSGSGKDF TLTITSLQTE DVATYYCQQY WSTPWTFGGG TKLEIK | 106 |
| light chain | | |
| SEQ ID NO: 227 | VGGYYDSMDH | 10 |
| 2155 heavy chain | | |
| CDR3 | | |
| SEQ ID NO: 228 | IISTGGSTY | 9 |
| 2155 heavy chain | | |
| CDR2 | | |
| SEQ ID NO: 229 | GFTISSYAMS | 10 |
| 2155 heavy chain | | |
| CDR1 | | |
| SEQ ID NO: 230 | QQSKEVPWT | 9 |
| 2155 light chain | | |
| CDR3 | | |
| SEQ ID NO: 231 | AASNQGS | 7 |
| 2155 light chain | | |
| CDR2 | | |
| SEQ ID NO: 232 | RASETVDNYG ISFMN | 15 |
| 2155 light chain | | |
| CDR1 | | |
| SEQ ID NO: 233 | TSTYPHFDY | 9 |
| 698 and 706 | | |
| heavy chain CDR3 | | |
| SEQ ID NO: 234 | AIYPGNSDTG | 10 |
| 698 and 706 | | |
| heavy chain CDR2 | | |
| SEQ ID NO: 235 | GYSFTTYWMH | 10 |
| 698 and 706 | | |
| heavy chain CDR1 | | |
| SEQ ID NO: 236 | QQSNNWPLT | 9 |
| 698 light chain | | |
| CDR3 | | |
| SEQ ID NO: 237 | KYASESIS | 8 |
| 698, 706, 827, | | |
| and 1649 light | | |
| chain CDR2 | | |
| SEQ ID NO: 238 | RASQSIGTSI H | 11 |
| 698, 706, 827, | | |
| and 1649 light | | |
| chain CDR1 | | |
| SEQ ID NO: 239 | QQTNNWPLT | 9 |
| 706, 827, and | | |
| 1649 light chain | | |
| CDR3 | | |
| SEQ ID NO: 240 | SSTYPHFDY | 9 |
| 827 and 1649 | | |
| heavy chain CDR3 | | |
| SEQ ID NO: 241 | TIYPGNSDAG | 10 |
| 827 heavy chain | | |
| CDR2 | | |
| SEQ ID NO: 242 | AIYPGNSDAG | 10 |
| 1649 heavy chain | | |
| CDR2 | | |
| SEQ ID NO: 243 | GYGNYYFPY | 9 |
| 1718 heavy chain | | |
| CDR3 | | |
| SEQ ID NO: 244 | WIYPGKGYTN | 10 |
| 1718 heavy chain | | |
| CDR2 | | |
| SEQ ID NO: 245 | DYTFTNYYI | 9 |
| 1718 heavy chain | | |
| CDR1 | | |
| SEQ ID NO: 246 | QQTWSTPWT | 9 |
| 1718 light chain | | |
| CDR3 | | |
| SEQ ID NO: 247 | AATSLET | 7 |
| 1718 light chain | | |
| CDR2 | | |
| SEQ ID NO: 248 | KASDHIKNWL A | 11 |
| 1718 light chain | | |
| CDR1 | | |
| SEQ ID NO: 249 | GYSFTTYWIH | 10 |
| 827 and 1649 | | |
| heavy chain CDR1 | | |

In a preferred embodiment, the GITR agonist is the monoclonal antibody 1D7, or a fragment, derivative, variant, or biosimilar thereof. 1D7 is available from Amgen, Inc. The preparation and properties of 1D7 are described in U.S. Patent Application Publication No. US 2015/0064204 A1, the disclosures of which are incorporated by reference herein. The amino acid sequences of 1D7 are set forth in Table 23.

In an embodiment, a GITR agonist comprises a heavy chain given by SEQ ID NO:250 and a light chain given by SEQ ID NO:251. In an embodiment, a GITR agonist comprises heavy and light chains having the sequences shown in SEQ ID NO:250 and SEQ ID NO:251, respectively, or antigen binding fragments, Fab fragments, single-chain variable fragments (scFv), variants, or conjugates thereof. In an embodiment, a GITR agonist comprises heavy and light chains that are each at least 99% identical to the sequences shown in SEQ ID NO:250 and SEQ ID NO:251, respectively. In an embodiment, a GITR agonist comprises heavy and light chains that are each at least 98% identical to the sequences shown in SEQ ID NO:250 and SEQ ID NO:251, respectively. In an embodiment, a GITR agonist comprises heavy and light chains that are each at least 97% identical to the sequences shown in SEQ ID NO:250 and SEQ ID NO:251, respectively. In an embodiment, a GITR agonist comprises heavy and light chains that are each at least 96% identical to the sequences shown in SEQ ID NO:250 and SEQ ID NO:251, respectively. In an embodiment, a GITR agonist comprises heavy and light chains that are each at least 95% identical to the sequences shown in SEQ ID NO:250 and SEQ ID NO:251, respectively.

In an embodiment, the GITR agonist comprises the heavy and light chain CDRs or variable regions (VRs) of 1D7. In an embodiment, the GITR agonist heavy chain variable region (V_{H}) comprises the sequence shown in SEQ ID NO:252, and the GITR agonist light chain variable region (V_{L}) comprises the sequence shown in SEQ ID NO:253, and conservative amino acid substitutions thereof. In an embodiment, a GITR agonist comprises V_{H} and V_{L} regions that are each at least 99% identical to the sequences shown in SEQ ID NO:252 and SEQ ID NO:253, respectively. In an embodiment, a GITR agonist comprises V_{H} and V_{L} regions that are each at least 98% identical to the sequences shown in SEQ ID NO:252 and SEQ ID NO:253, respectively. In an embodiment, a GITR agonist comprises V_{H} and V_{L} regions that are each at least 97% identical to the sequences shown in SEQ ID NO:252 and SEQ ID NO:253, respectively. In an embodiment, a GITR agonist comprises V_{H} and V_{L} regions that are each at least 96% identical to the sequences shown in SEQ ID NO:252 and SEQ ID NO:253, respectively. In an embodiment, a GITR agonist comprises V_{H} and V_{L} regions that are each at least 95% identical to the sequences shown in SEQ ID NO:252 and SEQ ID NO:253, respectively.

In an embodiment, a GITR agonist comprises heavy chain CDR1, CDR2 and CDR3 domains having the sequences set forth in SEQ ID NO:254, SEQ ID NO:255, and SEQ ID NO:256, respectively, and conservative amino acid substitutions thereof, and light chain CDR1, CDR2 and CDR3 domains having the sequences set forth in SEQ ID NO:257, SEQ ID NO:258, and SEQ ID NO:259, respectively, and conservative amino acid substitutions thereof.

In an embodiment, the GITR agonist is a GITR agonist biosimilar monoclonal antibody approved by drug regulatory authorities with reference to 1D7. In an embodiment, the biosimilar monoclonal antibody comprises an GITR antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 1D7. In some embodiments, the one or more post-Attorney translational modifications are selected from one or more of: glycosylation, oxidation, deamidation, and truncation. In some embodiments, the biosimilar is a GITR agonist antibody authorized or submitted for authorization, wherein the GITR agonist antibody is provided in a formulation which differs from the formulations of a reference medicinal product or reference biological product, wherein the reference medicinal product or reference biological product is 1D7. The GITR agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 1D7. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 1D7.

TABLE 23
Amino acid sequences for GITR agonist antibodies related to 1D7.

| Identi- | | |
|---|---|---|
| fier | Sequence (One-Letter Amino Acid Symbols) | |
| SEQ ID | QVQLVESGGG VVQPGRSLRL SCAASGFTFS SYGMHWVRQA PGKGLEWVTV IWYEGSNKYY | 60 |
| NO: 250 | ADSVKGRFTI SRDNSKNTLY LQMNSLRAED TAVYYCARGG QLGKYYYYGM DVWGQGTTVT | 120 |
| 1D7 | VSSASTKGPS VFPLAPSSKS TSGGTAALGC LVKDYFPEPV TVSWNSGALT SGVHTFPAVL | 180 |
| heavy | QSSGLYSLSS VVTVPSSSLG TQTYICNVNH KPSNTKVDKR VEPKSCDKTH TCPPCPAPEL | 240 |
| chain | LGGPSVFLFP PKPKDTLMIS RTPEVTCVVV DVSHEDPEVK FNWYVDGVEV HNAKTKPREE | 300 |
| | QYNSTYRVVS VLTVLHQDWL NGKEYKCKVS NKALPAPIEK TISKAKGQPR EPQVYTLPPS | 360 |
| | REEMTKNQVS LTCLVKGFYP SDIAVEWESN GQPENNYKTT PPVLDSDGSF FLYSKLTVDK | 420 |
| | SRWQQGNVFS CSVMHEALHN HYTQKSLSLS PGK | 453 |
| SEQ ID | DIQMTQSPSS LSASVGDRVT ITCRASQGIR NDLGWYQQKP GKAPKRLIYD ASSLQSGVPS | 60 |
| NO: 251 | RFSGSGSGTE FTLTISSLQP EDFATYYCLQ HNNYPWTFGQ GTKVEIKRTV AAPSVFIFPP | 120 |
| 1D7 | SDEQLKSGTA SVVCLLNNFY PREAKVQWKV DNALQSGNSQ ESVTEQDSKD STYSLSSTLT | 180 |
| light | LSKADYEKHK VYACEVTHQG LSSPVTKSFN RGEC | 214 |
| chain | | |
| SEQ ID | QVQLVESGGG VVQPGRSLRL SCAASGFTFS SYGMHWVRQA PGKGLEWVTV IWYEGSNKYY | 60 |
| NO: 252 | ADSVKGRFTI SRDNSKNTLY LQMNSLRAED TAVYYCARGG QLGKYYYYGM DVWGQGTTVT | 120 |
| 1D7 | VSS | 123 |
| variable | | |
| heavy | | |
| chain | | |
| SEQ ID | DIQMTQSPSS LSASVGDRVT ITCRASQGIR NDLGWYQQKP GKAPKRLIYD ASSLQSGVPS | 60 |
| NO: 253 | RFSGSGSGTE FTLTISSLQP EDFATYYCLQ HNNYPWTFGQ GTKVEIKR | 108 |
| 1D7 | | |
| variable | | |
| light | | |
| chain | | |
| SEQ ID | SYGMH | 5 |
| NO: 254 | | |
| 1D7 | | |
| heavy | | |
| chain | | |
| CDR1 | | |
| SEQ ID | VIWYEGSNKY YADSVKG | 17 |
| NO: 255 | | |
| 1D7 | | |
| heavy | | |
| chain | | |
| CDR2 | | |
| SEQ ID | GGQLGKYYYY GMDV | 14 |
| NO: 256 | | |
| 1D7 | | |
| heavy | | |
| chain | | |
| CDR3 | | |
| SEQ ID | RASQGIRNDL G | 11 |
| NO: 257 | | |
| 1D7 | | |
| light | | |
| chain | | |
| CDR1 | | |
| SEQ ID | DASSLQS | 7 |
| NO: 258 | | |
| 1D7 | | |
| light | | |
| chain | | |
| CDR2 | | |
| SEQ ID | LQHNNYPWT | 9 |
| NO: 259 | | |
| 1D7 | | |
| light | | |
| chain | | |
| CDR3 | | |

In a preferred embodiment, the GITR agonist is the monoclonal antibody 33C9, or a fragment, derivative, variant, or biosimilar thereof. 33C9 is available from Amgen, Inc. The preparation and properties of 33C9 are described in U.S. Patent Application Publication No. US 2015/0064204 A1, the disclosures of which are incorporated by reference herein. The amino acid sequences of 33C9 are set forth in Table 24.

In an embodiment, a GITR agonist comprises a heavy chain given by SEQ ID NO:260 and a light chain given by SEQ ID NO:261. In an embodiment, a GITR agonist comprises heavy and light chains having the sequences shown in SEQ ID NO:260 and SEQ ID NO:261, respectively, or antigen binding fragments, Fab fragments, single-chain variable fragments (scFv), variants, or conjugates thereof. In an embodiment, a GITR agonist comprises heavy and light chains that are each at least 99% identical to the sequences shown in SEQ ID NO:260 and SEQ ID NO:261, respectively. In an embodiment, a GITR agonist comprises heavy and light chains that are each at least 98% identical to the sequences shown in SEQ ID NO:260 and SEQ ID NO:261, respectively. In an embodiment, a GITR agonist comprises heavy and light chains that are each at least 97% identical to the sequences shown in SEQ ID NO:260 and SEQ ID NO:261, respectively. In an embodiment, a GITR agonist comprises heavy and light chains that are each at least 96% identical to the sequences shown in SEQ ID NO:260 and SEQ ID NO:261, respectively. In an embodiment, a GITR agonist comprises heavy and light chains that are each at least 95% identical to the sequences shown in SEQ ID NO:260 and SEQ ID NO:261, respectively.

In an embodiment, the GITR agonist comprises the heavy and light chain CDRs or variable regions (VRs) of 1D7. In an embodiment, the GITR agonist heavy chain variable region (V_{H}) comprises the sequence shown in SEQ ID NO:262, and the GITR agonist light chain variable region (V_{L}) comprises the sequence shown in SEQ ID NO:263, and conservative amino acid substitutions thereof. In an embodiment, a GITR agonist comprises V_{H} and V_{L} regions that are each at least 99% identical to the sequences shown in SEQ ID NO:262 and SEQ ID NO:263, respectively. In an embodiment, a GITR agonist comprises V_{H} and V_{L} regions that are each at least 98% identical to the sequences shown in SEQ ID NO:262 and SEQ ID NO:263, respectively. In an embodiment, a GITR agonist comprises V_{H} and V_{L} regions that are each at least 97% identical to the sequences shown in SEQ ID NO:262 and SEQ ID NO:263, respectively. In an embodiment, a GITR agonist comprises V_{H} and V_{L} regions that are each at least 96% identical to the sequences shown in SEQ ID NO:262 and SEQ ID NO:263, respectively. In an embodiment, a GITR agonist comprises V_{H} and V_{L} regions that are each at least 95% identical to the sequences shown in SEQ ID NO:262 and SEQ ID NO:263, respectively.

In an embodiment, a GITR agonist comprises heavy chain CDR1, CDR2 and CDR3 domains having the sequences set forth in SEQ ID NO:264, SEQ ID NO:265, and SEQ ID NO:266, respectively, and conservative amino acid substitutions thereof, and light chain CDR1, CDR2 and CDR3 domains having the sequences set forth in SEQ ID NO:267, SEQ ID NO:268, and SEQ ID NO:269, respectively, and conservative amino acid substitutions thereof.

In an embodiment, the GITR agonist is a GITR agonist biosimilar monoclonal antibody approved by drug regulatory authorities with reference to 33C9. In an embodiment, the biosimilar monoclonal antibody comprises an GITR antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 33C9. In some embodiments, the one or more post-translational modifications are selected from one or more of: glycosylation, oxidation, deamidation, and truncation. In some embodiments, the biosimilar is a GITR agonist antibody authorized or submitted for authorization, wherein the GITR agonist antibody is provided in a formulation which differs from the formulations of a reference medicinal product or reference biological product, wherein the reference medicinal product or reference biological product is 33C9. The GITR agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 33C9. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 33C9.

TABLE 24
Amino acid sequences for GITR agonist antibodies related to 33C9.

| Identi- | | |
|---|---|---|
| fier | Sequence (One-Letter Amino Acid Symbols) | |
| SEQ ID | QVQVVESGGG VVQPGRSLRL SCAASGFTFS SYGMHWVRQA PGKGLEWVSV IWYEGSNKYY | 60 |
| NO: 260 | ADSVKGRFTI SRDNSKNTLY LQMNSLRAED TAVYYCARGG LLGYYYYYGM DVWGQGTTVT | 120 |
| 33C9 | VSSASTKGPS VFPLAPSSKS TSGGTAALGC LVKDYFPEPV TVSWNSGALT SGVHTFPAVL | 180 |
| heavy | QSSGLYSLSS VVTVPSSSLG TQTYICNVNH KPSNTKVDKK VEPKSCDKTH TCPPCPAPEL | 240 |
| chain | LGGPSVFLFP PKPKDTLMIS RTPEVTCVVV DVSHEDPEVK FNWYVDGVEV HNAKTKPREE | 300 |
| | QYNSTYRVVS VLTVLHQDWL NGKEYKCKVS NKALPAPIEK TISKAKGQPR EPQVYTLPPS | 360 |
| | REEMTKNQVS LTCLVKGFYP SDIAVEWESN GQPENNYKTT PPVLDSDGSF FLYSKLTVDK | 420 |
| | SRWQQGNVFS CSVMHEALHN HYTQKSLSLS PGK | 453 |
| SEQ ID | DIQMTQSPSS LSASVGDRVT ITCRASQGIR NDLGWYQQKP GKAPKRLIYD ASSLQSGVPS | 60 |
| NO: 261 | RFSGSGSGTE FTLTISSLQP EDFATYYCLQ HHSYPWTFGQ GTKVEIKRTV AAPSVFIFPP | 120 |
| 33C9 | SDEQLKSGTA SVVCLLNNFY PREAKVQWKV DNALQSGNSQ ESVTEQDSKD STYSLSSTLT | 160 |
| light | LSKADYEKHK VYACEVTHQG LSSPVTKSFN RGEC | 214 |
| chain | | |
| SEQ ID | QVQVVESGGG VVQPGRSLRL SCAASGFTFS SYGMHWVRQA PGKGLEWVSV IWYEGSNKYY | 60 |
| NO: 262 | ADSVKGRFTI SRDNSKNTLY LQMNSLRAED TAVYYCARGG LLGYYYYYGM DVWGQGTTVT | 120 |
| 33C9 | VSS | 123 |
| variable | | |
| heavy | | |
| chain | | |
| SEQ ID | DIQMTQSPSS LSASVGDRVT ITCRASQGIR NDLGWYQQKP GKAPKRLIYD ASSLQSGVPS | 60 |
| NO: 263 | RFSGSGSGTE FTLTISSLQP EDFATYYCLQ HHSYPWTFGQ GTKVEIKR | 108 |
| 33C9 | | |
| variable | | |
| light | | |
| chain | | |
| SEQ ID | SYGMH | 5 |
| NO: 264 | | |
| 33C9 | | |
| heavy | | |
| chain | | |
| CDR1 | | |
| SEQ ID | VIWYEGSNKY YADSVKG | 17 |
| NO: 265 | | |
| 33C9 | | |
| heavy | | |
| chain | | |
| CDR2 | | |
| SEQ ID | GGLLGYYYYY GMDV | 14 |
| NO: 266 | | |
| 33C9 | | |
| heavy | | |
| chain | | |
| CDR3 | | |
| SEQ ID | RASQGIRNDL G | 11 |
| NO: 267 | | |
| 33C9 | | |
| light | | |
| chain | | |
| CDR1 | | |
| SEQ ID | DASSLQS | 7 |
| NO: 268 | | |
| 33C9 | | |
| light | | |
| chain | | |
| CDR2 | | |
| SEQ ID | LQHHSYPWT | 9 |
| NO: 269 | | |
| 33C9 | | |
| light | | |
| chain | | |
| CDR3 | | |

In a preferred embodiment, the GITR agonist is the monoclonal antibody 33F6, or a fragment, derivative, variant, or biosimilar thereof. 33F6 is available from Amgen, Inc. The preparation and properties of 33F6 are described in U.S. Patent Application Publication No. US 2015/0064204 A1, the disclosures of which are incorporated by reference herein. The amino acid sequences of 33F6 are set forth in Table 25.

In an embodiment, a GITR agonist comprises a heavy chain given by SEQ ID NO:270 and a light chain given by SEQ ID NO:271. In an embodiment, a GITR agonist comprises heavy and light chains having the sequences shown in SEQ ID NO:270 and SEQ ID NO:271, respectively, or antigen binding fragments, Fab fragments, single-chain variable fragments (scFv), variants, or conjugates thereof. In an embodiment, a GITR agonist comprises heavy and light chains that are each at least 99% identical to the sequences shown in SEQ ID NO:270 and SEQ ID NO:271, respectively. In an embodiment, a GITR agonist comprises heavy and light chains that are each at least 98% identical to the sequences shown in SEQ ID NO:270 and SEQ ID NO:271, respectively. In an embodiment, a GITR agonist comprises heavy and light chains that are each at least 97% identical to the sequences shown in SEQ ID NO:270 and SEQ ID NO:271, respectively. In an embodiment, a GITR agonist comprises heavy and light chains that are each at least 96% identical to the sequences shown in SEQ ID NO:270 and SEQ ID NO:271, respectively. In an embodiment, a GITR agonist comprises heavy and light chains that are each at least 95% identical to the sequences shown in SEQ ID NO:270 and SEQ ID NO:271, respectively.

In an embodiment, the GITR agonist comprises the heavy and light chain CDRs or variable regions (VRs) of 33F6. In an embodiment, the GITR agonist heavy chain variable region (V_{H}) comprises the sequence shown in SEQ ID NO:272, and the GITR agonist light chain variable region (V_{L}) comprises the sequence shown in SEQ ID NO:273, and conservative amino acid substitutions thereof. In an embodiment, a GITR agonist comprises V_{H} and V_{L} regions that are each at least 99% identical to the sequences shown in SEQ ID NO:272 and SEQ ID NO:273, respectively. In an embodiment, a GITR agonist comprises V_{H} and V_{L} regions that are each at least 98% identical to the sequences shown in SEQ ID NO:272 and SEQ ID NO:273, respectively. In an embodiment, a GITR agonist comprises V_{H} and V_{L} regions that are each at least 97% identical to the sequences shown in SEQ ID NO:272 and SEQ ID NO:273, respectively. In an embodiment, a GITR agonist comprises V_{H} and V_{L} regions that are each at least 96% identical to the sequences shown in SEQ ID NO:272 and SEQ ID NO:273, respectively. In an embodiment, a GITR agonist comprises V_{H} and V_{L} regions that are each at least 95% identical to the sequences shown in SEQ ID NO:272 and SEQ ID NO:273, respectively.

In an embodiment, a GITR agonist comprises heavy chain CDR1, CDR2 and CDR3 domains having the sequences set forth in SEQ ID NO:274, SEQ ID NO:275, and SEQ ID NO:276, respectively, and conservative amino acid substitutions thereof, and light chain CDR1, CDR2 and CDR3 domains having the sequences set forth in SEQ ID NO:277, SEQ ID NO:278, and SEQ ID NO:279, respectively, and conservative amino acid substitutions thereof.

In an embodiment, the GITR agonist is a GITR agonist biosimilar monoclonal antibody approved by drug regulatory authorities with reference to 33F6. In an embodiment, the biosimilar monoclonal antibody comprises an GITR antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 33F6. In some embodiments, the one or more post-translational modifications are selected from one or more of: glycosylation, oxidation, deamidation, and truncation. In some embodiments, the biosimilar is a GITR agonist antibody authorized or submitted for authorization, wherein the GITR agonist antibody is provided in a formulation which differs from the formulations of a reference medicinal product or reference biological product, wherein the reference medicinal product or reference biological product is 33F6. The GITR agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 33F6. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 33F6.

TABLE 25
Amino acid sequences for GITR agonist antibodies related to 33F6.

| Identi- | | |
|---|---|---|
| fier | Sequence (One-Letter Amino Acid Symbols) | |
| SEQ ID | QVQLVESGGG VVQPGRSLRL SCAASGFTFS NYGMHWVRQA PGKGLEWVAV IWYVGSNKYY | 60 |
| NO: 270 | ADSVKGRFTI SRDNSKNTLY LQMNSLRAED TAVYYCARGG ELRLYYYYGM DVWGQGTTVT | 120 |
| 33F6 | VSSASTKGPS VFPLAPSSKS TSGGTAALGC LVKDYFPEPV TVSWNSGALT SGVHTFPAVL | 180 |
| heavy | QSSGLYSLSS VVTVPSSSLG TQTYICNVNH KPSNTKVDKK VEPKSCDKTH TCPPCPAPEL | 240 |
| chain | LGGPSVFLFP PKPKDTLMIS RTPEVTCVVV DVSHEDPEVK FNWYVDGVEV HNAKTKPREE | 300 |
| | QYNSTYRVVS VLTVLHQDWL NGKEYKCKVS NKALPAPIEK TISKAKGQPR EPQVYTLPPS | 360 |
| | REEMTKNQVS LTCLVKGFYP SDIAVEWESN GQPENNYKTT PPVLDSDGSF FLYSKLTVDK | 420 |
| | SRWQQGNVFS CSVMHEALHN HYTQKSLSLS PGK | 453 |
| SEQ ID | DIQMTQSPSS LSASVGDRVT ITCRASQGIR NDLGWYQQKP GKAPKRLIYA ASSLQSGVPS | 60 |
| NO: 271 | RFSGSGSGTE FTLTVSSLQP EDFATYYCLQ LNSYPWTFGQ GTKVEIKRTV AAPSVFIFPP | 120 |
| 33F6 | SDEQLKSGTA SVVCLLNNFY PREAKVQWKV DNALQSGNSQ ESVTEQDSKD STYSLSSTLT | 160 |
| light | LSKADYEKHK VYACEVTHQG LSSPVTKSFN RGEC | 214 |
| chain | | |
| SEQ ID | QVQLVESGGG VVQPGRSLRL SCAASGFTFS NYGMHWVRQA PGKGLEWVAV IWYVGSNKYY | 60 |
| NO: 272 | ADSVKGRFTI SRDNSKNTLY LQMNSLRAED TAVYYCARGG ELRLYYYYGM DVWGQGTTVT | 120 |
| 33F6 | VSS | 123 |
| variable | | |
| heavy | | |
| chain | | |
| SEQ ID | DIQMTQSPSS LSASVGDRVT ITCRASQGIR NDLGWYQQKP GKAPKRLIYA ASSLQSGVPS | 60 |
| NO: 273 | RFSGSGSGTE FTLTVSSLQP EDFATYYCLQ LNSYPWTFGQ GTKVEIKR | 108 |
| 33F6 | | |
| variable | | |
| light | | |
| chain | | |
| SEQ ID | NYGMH | 5 |
| NO: 274 | | |
| 33F6 | | |
| heavy | | |
| chain | | |
| CDR1 | | |
| SEQ ID | VIWYVGSNKY YADSVKG | 17 |
| NO: 275 | | |
| 33F6 | | |
| heavy | | |
| chain | | |
| CDR2 | | |
| SEQ ID | GGELRLYYYY GMDV | 14 |
| NO: 276 | | |
| 33F6 | | |
| heavy | | |
| chain | | |
| CDR3 | | |
| SEQ ID | RASQGIRNDL G | 11 |
| NO: 277 | | |
| 33F6 | | |
| light | | |
| chain | | |
| CDR1 | | |
| SEQ ID | AASSLQS | 7 |
| NO: 278 | | |
| 33F6 | | |
| light | | |
| chain | | |
| CDR2 | | |
| SEQ ID | LQLNSYPWT | 9 |
| NO: 279 | | |
| 33F6 | | |
| light | | |
| chain | | |
| CDR3 | | |

In a preferred embodiment, the GITR agonist is the monoclonal antibody 34G4, or a fragment, derivative, variant, or biosimilar thereof. 34G4 is available from Amgen, Inc. The preparation and properties of 34G4 are described in U.S. Patent Application Publication No. US 2015/0064204 A1, the disclosures of which are incorporated by reference herein. The amino acid sequences of 34G4 are set forth in Table 26.

In an embodiment, a GITR agonist comprises a heavy chain given by SEQ ID NO:280 and a light chain given by SEQ ID NO:281. In an embodiment, a GITR agonist comprises heavy and light chains having the sequences shown in SEQ ID NO:280 and SEQ ID NO:281, respectively, or antigen binding fragments, Fab fragments, single-chain variable fragments (scFv), variants, or conjugates thereof. In an embodiment, a GITR agonist comprises heavy and light chains that are each at least 99% identical to the sequences shown in SEQ ID NO:280 and SEQ ID NO:281, respectively. In an embodiment, a GITR agonist comprises heavy and light chains that are each at least 98% identical to the sequences shown in SEQ ID NO:280 and SEQ ID NO:281, respectively. In an embodiment, a GITR agonist comprises heavy and light chains that are each at least 97% identical to the sequences shown in SEQ ID NO:280 and SEQ ID NO:281, respectively. In an embodiment, a GITR agonist comprises heavy and light chains that are each at least 96% identical to the sequences shown in SEQ ID NO:280 and SEQ ID NO:281, respectively. In an embodiment, a GITR agonist comprises heavy and light chains that are each at least 95% identical to the sequences shown in SEQ ID NO:280 and SEQ ID NO:281, respectively.

In an embodiment, the GITR agonist comprises the heavy and light chain CDRs or variable regions (VRs) of 34G4. In an embodiment, the GITR agonist heavy chain variable region (V_{H}) comprises the sequence shown in SEQ ID NO:282, and the GITR agonist light chain variable region (V_{L}) comprises the sequence shown in SEQ ID NO:283, and conservative amino acid substitutions thereof. In an embodiment, a GITR agonist comprises V_{H} and V_{L} regions that are each at least 99% identical to the sequences shown in SEQ ID NO:282 and SEQ ID NO:283, respectively. In an embodiment, a GITR agonist comprises V_{H} and V_{L} regions that are each at least 98% identical to the sequences shown in SEQ ID NO:282 and SEQ ID NO:283, respectively. In an embodiment, a GITR agonist comprises V_{H} and V_{L} regions that are each at least 97% identical to the sequences shown in SEQ ID NO:282 and SEQ ID NO:283, respectively. In an embodiment, a GITR agonist comprises V_{H} and V_{L} regions that are each at least 96% identical to the sequences shown in SEQ ID NO:282 and SEQ ID NO:283, respectively. In an embodiment, a GITR agonist comprises V_{H} and V_{L} regions that are each at least 95% identical to the sequences shown in SEQ ID NO:282 and SEQ ID NO:283, respectively.

In an embodiment, a GITR agonist comprises heavy chain CDR1, CDR2 and CDR3 domains having the sequences set forth in SEQ ID NO:284, SEQ ID NO:285, and SEQ ID NO:286, respectively, and conservative amino acid substitutions thereof, and light chain CDR1, CDR2 and CDR3 domains having the sequences set forth in SEQ ID NO:287, SEQ ID NO:288, and SEQ ID NO:289, respectively, and conservative amino acid substitutions thereof.

In an embodiment, the GITR agonist is a GITR agonist biosimilar monoclonal antibody approved by drug regulatory authorities with reference to 34G4. In an embodiment, the biosimilar monoclonal antibody comprises an GITR antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 34G4. In some embodiments, the one or more post-translational modifications are selected from one or more of: glycosylation, oxidation, deamidation, and truncation. In some embodiments, the biosimilar is a GITR agonist antibody authorized or submitted for authorization, wherein the GITR agonist antibody is provided in a formulation which differs from the formulations of a reference medicinal product or reference biological product, wherein the reference medicinal product or reference biological product is 34G4. The GITR agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 34G4. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 34G4.

TABLE 26
Amino acid sequences for GITR agonist antibodies related to 34G4.

| Identi- | | |
|---|---|---|
| fier | Sequence (One-Letter Amino Acid Symbols) | |
| SEQ ID | QVQLVESGGG VVQPGRSLRL SCAASGFTFS SYGMHWVRQA PGKGLEWVAV IWYEGSNKYY | 60 |
| NO: 280 | ADSVKGRFTI SRDNSKNTLY LQMNSLRAED TAVYYCARGG QLGYYYYYGM DVWGQGTTVT | 120 |
| 34G4 | VSSASTKGPS VFPLAPSSKS TSGGTAALGC LVKDYFPEPV TVSWNSGALT SGVHTFPAVL | 180 |
| heavy | QSSGLYSLSS VVTVPSSSLG TQTYICNVNH KPSNTKVDKK VEPKSCDKTH TCPPCPAPEL | 240 |
| chain | LGGPSVFLFP PKPKDTLMIS RTPEVTCVVV DVSHEDPEVK FNWYVDGVEV HNAKTKPREE | 300 |
| | QYNSTYRVVS VLTVLHQDWL NGKEYKCKVS NKALPAPIEK TISKAKGQPR EPQVYTLPPS | 360 |
| | REEMTKNQVS LTCLVKGFYP SDIAVEWESN GQPENNYKTT PPVLDSDGSF FLYSKLTVDK | 420 |
| | SRWQQGNVFS CSVMHEALHN HYTQKSLSLS PGK | 453 |
| SEQ ID | DIQMTQSPSS LSASVGDRVT ITCRASQGIR NDLGWYQQKP GKAPKRLIYD ASSLQSGVPS | 60 |
| NO: 281 | RFSGSGSGTD FTLTISSLQP EDFATYYCLQ LNSYPWTFGQ GTKVEIKRTV AAPSVFIFPP | 120 |
| 34G4 | SDEQLKSGTA SVVCLLNNFY PREAKVQWKV DNALQSGNSQ ESVTEQDSKD STYSLSSTLT | 160 |
| light | LSKADYEKHK VYACEVTHQG LSSPVTKSFN RGEC | 214 |
| chain | | |
| SEQ ID | QVQLVESGGG VVQPGRSLRL SCAASGFTFS SYGMHWVRQA PGKGLEWVAV IWYEGSNKYY | 60 |
| NO: 282 | ADSVKGRFTI SRDNSKNTLY LQMNSLRAED TAVYYCARGG QLGYYYYYGM DVWGQGTTVT | 120 |
| 34G4 | VSS | 123 |
| variable | | |
| heavy | | |
| chain | | |
| SEQ ID | DIQMTQSPSS LSASVGDRVT ITCRASQGIR NDLGWYQQKP GKAPKRLIYD ASSLQSGVPS | 60 |
| NO: 283 | RFSGSGSGTD FTLTISSLQP EDFATYYCLQ LNSYPWTFGQ GTKVEIKR | 108 |
| 34G4 | | |
| variable | | |
| light | | |
| chain | | |
| SEQ ID | SYGMH | 5 |
| NO: 284 | | |
| 34G4 | | |
| heavy | | |
| chain | | |
| CDR1 | | |
| SEQ ID | VIWYEGSNKY YADSVKG | 17 |
| NO: 285 | | |
| 34G4 | | |
| heavy | | |
| chain | | |
| CDR2 | | |
| SEQ ID | GGQLGYYYYY GMDV | 14 |
| NO: 286 | | |
| 34G4 | | |
| heavy | | |
| chain | | |
| CDR3 | | |
| SEQ ID | RASQGIRNDL G | 11 |
| NO: 287 | | |
| 34G4 | | |
| light | | |
| chain | | |
| CDR1 | | |
| SEQ ID | DASSLQS | 7 |
| NO: 288 | | |
| 34G4 | | |
| light | | |
| chain | | |
| CDR2 | | |
| SEQ ID | LQLNSYPWT | 9 |
| NO: 289 | | |
| 34G4 | | |
| light | | |
| chain | | |
| CDR3 | | |

In a preferred embodiment, the GITR agonist is the monoclonal antibody 35B10, or a fragment, derivative, variant, or biosimilar thereof 35B10 is available from Amgen, Inc. The preparation and properties of 35B10 are described in U.S. Patent Application Publication No. US 2015/0064204 A1, the disclosures of which are incorporated by reference herein. The amino acid sequences of 35B10 are set forth in Table 27.

In an embodiment, a GITR agonist comprises a heavy chain given by SEQ ID NO:290 and a light chain given by SEQ ID NO:291. In an embodiment, a GITR agonist comprises heavy and light chains having the sequences shown in SEQ ID NO:290 and SEQ ID NO:291, respectively, or antigen binding fragments, Fab fragments, single-chain variable fragments (scFv), variants, or conjugates thereof. In an embodiment, a GITR agonist comprises heavy and light chains that are each at least 99% identical to the sequences shown in SEQ ID NO:290 and SEQ ID NO:291, respectively. In an embodiment, a GITR agonist comprises heavy and light chains that are each at least 98% identical to the sequences shown in SEQ ID NO:290 and SEQ ID NO:291, respectively. In an embodiment, a GITR agonist comprises heavy and light chains that are each at least 97% identical to the sequences shown in SEQ ID NO:290 and SEQ ID NO:291, respectively. In an embodiment, a GITR agonist comprises heavy and light chains that are each at least 96% identical to the sequences shown in SEQ ID NO:290 and SEQ ID NO:291, respectively. In an embodiment, a GITR agonist comprises heavy and light chains that are each at least 95% identical to the sequences shown in SEQ ID NO:290 and SEQ ID NO:291, respectively.

In an embodiment, the GITR agonist comprises the heavy and light chain CDRs or variable regions (VRs) of 35B10. In an embodiment, the GITR agonist heavy chain variable region (V_{H}) comprises the sequence shown in SEQ ID NO:292, and the GITR agonist light chain variable region (V_{L}) comprises the sequence shown in SEQ ID NO:293, and conservative amino acid substitutions thereof. In an embodiment, a GITR agonist comprises V_{H} and V_{L} regions that are each at least 99% identical to the sequences shown in SEQ ID NO:292 and SEQ ID NO:293, respectively. In an embodiment, a GITR agonist comprises V_{H} and V_{L} regions that are each at least 98% identical to the sequences shown in SEQ ID NO:292 and SEQ ID NO:293, respectively. In an embodiment, a GITR agonist comprises V_{H} and V_{L} regions that are each at least 97% identical to the sequences shown in SEQ ID NO:292 and SEQ ID NO:293, respectively. In an embodiment, a GITR agonist comprises V_{H} and V_{L} regions that are each at least 96% identical to the sequences shown in SEQ ID NO:292 and SEQ ID NO:293, respectively. In an embodiment, a GITR agonist comprises V_{H} and V_{L} regions that are each at least 95% identical to the sequences shown in SEQ ID NO:292 and SEQ ID NO:293, respectively.

In an embodiment, a GITR agonist comprises heavy chain CDR1, CDR2 and CDR3 domains having the sequences set forth in SEQ ID NO:294, SEQ ID NO:295, and SEQ ID NO:296, respectively, and conservative amino acid substitutions thereof, and light chain CDR1, CDR2 and CDR3 domains having the sequences set forth in SEQ ID NO:297, SEQ ID NO:298, and SEQ ID NO:299, respectively, and conservative amino acid substitutions thereof.

In an embodiment, the GITR agonist is a GITR agonist biosimilar monoclonal antibody approved by drug regulatory authorities with reference to 35B10. In an embodiment, the biosimilar monoclonal antibody comprises an GITR antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 35B10. In some embodiments, the one or more post-translational modifications are selected from one or more of: glycosylation, oxidation, deamidation, and truncation. In some embodiments, the biosimilar is a GITR agonist antibody authorized or submitted for authorization, wherein the GITR agonist antibody is provided in a formulation which differs from the formulations of a reference medicinal product or reference biological product, wherein the reference medicinal product or reference biological product is 35B10. The GITR agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 35B 10. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 35B310.

TABLE 27
Amino acid sequences for GITR agonist antibodies related to 35B10.

| Identi- | | |
|---|---|---|
| fier | Sequence (One-Letter Amino Acid Symbols) | |
| SEQ ID | QVQLVESGGG VVQPGRSLRL SCAASGFTFS SYGMHWVRQA PGKGLEWVAV IWYAGSNKYY | 60 |
| NO: 290 | ADSVKGRFTI SRDNSKNTLY LQMNSLRAED TAVYYCARGG ELSFYYYYGM DVWGQGTTVT | 120 |
| 35B10 | VSSASTKGPS VFPLAPSSKS TSGGTAALGC LVKDYFPEPV TVSWNSGALT SGVHTFPAVL | 180 |
| heavy | QSSGLYSLSS VVTVPSSSLG TQTYICNVNH KPSNTKVDKK VEPKSCDKTH TCPPCPAPEL | 240 |
| chain | LGGPSVFLFP PKPKDTLMIS RTPEVTCVVV DVSHEDPEVK FNWYVDGVEV HNAKTKPREE | 300 |
| | QYNSTYRVVS VLTVLHQDWL NGKEYKCKVS NKALPAPIEK TISKAKGQPR EPQVYTLPPS | 360 |
| | REEMTKNQVS LTCLVKGFYP SDIAVEWESN GQPENNYKTT PPVLDSDGSF FLYSKLTVDK | 420 |
| | SRWQQGNVFS CSVMHEALHN HYTQKSLSLS PGK | 453 |
| SEQ ID | DIQMTQSPSS LSASVGDRVT ITCRASQGIR NDLGWYQQKP GKAPKRLIYA ASTLQSGVPS | 60 |
| NO: 291 | RFSGSGSGTE FTLTISSLQP EDFATYYCLQ HNNYPWTFGQ GTKVEIKRTV AAPSVFIFPP | 120 |
| 35B10 | SDEQLKSGTA SVVCLLNNFY PREAKVQWKV DNALQSGNSQ ESVTEQDSKD STYSLSSTLT | 100 |
| light | LSKADYEKHK VYACEVTHQG LSSPVTKSFN RGEC | 214 |
| chain | | |
| SEQ ID | QVQLVESGGG VVQPGRSLRL SCAASGFTFS SYGMHWVRQA PGKGLEWVAV IWYAGSNKYY | 60 |
| NO: 292 | ADSVKGRFTI SRDNSKNTLY LQMNSLRAED TAVYYCARGG ELSFYYYYGM DVWGQGTTVT | 120 |
| 35B10 | VSS | 123 |
| variable | | |
| heavy chain | | |
| SEQ ID | DIQMTQSPSS LSASVGDRVT ITCRASQGIR NDLGWYQQKP GKAPKRLIYA ASTLQSGVPS | 60 |
| NO: 293 | RFSGSGSGTE FTLTISSLQP EDFATYYCLQ HNNYPWTFGQ GTKVEIKR | 108 |
| 35B10 | | |
| variable | | |
| light chain | | |
| SEQ ID | SYGMH | 5 |
| NO: 294 | | |
| 35B10 | | |
| heavy chain | | |
| CDR1 | | |
| SEQ ID | VIWYAGSNKY YADSVKG | 17 |
| NO: 295 | | |
| 35B10 | | |
| heavy chain | | |
| CDR2 | | |
| SEQ ID | GGELSFYYYY GMDV | 14 |
| NO: 296 | | |
| 35B10 | | |
| heavy | | |
| chain | | |
| CDR3 | | |
| SEQ ID | RASQGIRNDL G | 11 |
| NO: 297 | | |
| 35B10 | | |
| light | | |
| chain | | |
| CDR1 | | |
| SEQ ID | AASTLQS | 7 |
| NO: 298 | | |
| 35B10 | | |
| light | | |
| chain | | |
| CDR2 | | |
| SEQ ID | LQHNNYPWT | 9 |
| NO: 299 | | |
| 35B10 | | |
| light | | |
| chain | | |
| CDR3 | | |

In a preferred embodiment, the GITR agonist is the monoclonal antibody 41E11, or a fragment, derivative, variant, or biosimilar thereof. 41E11 is available from Amgen, Inc. The preparation and properties of 41E11 are described in U.S. Patent Application Publication No. US 2015/0064204 A1, the disclosures of which are incorporated by reference herein. The amino acid sequences of 41E11 are set forth in Table 28.

In an embodiment, a GITR agonist comprises a heavy chain given by SEQ ID NO:300 and a light chain given by SEQ ID NO:301. In an embodiment, a GITR agonist comprises heavy and light chains having the sequences shown in SEQ ID NO:300 and SEQ ID NO:301, respectively, or antigen binding fragments, Fab fragments, single-chain variable fragments (scFv), variants, or conjugates thereof. In an embodiment, a GITR agonist comprises heavy and light chains that are each at least 99% identical to the sequences shown in SEQ ID NO:300 and SEQ ID NO:301, respectively. In an embodiment, a GITR agonist comprises heavy and light chains that are each at least 98% identical to the sequences shown in SEQ ID NO:300 and SEQ ID NO:301, respectively. In an embodiment, a GITR agonist comprises heavy and light chains that are each at least 97% identical to the sequences shown in SEQ ID NO:300 and SEQ ID NO:301, respectively. In an embodiment, a GITR agonist comprises heavy and light chains that are each at least 96% identical to the sequences shown in SEQ ID NO:300 and SEQ ID NO:301, respectively. In an embodiment, a GITR agonist comprises heavy and light chains that are each at least 95% identical to the sequences shown in SEQ ID NO:300 and SEQ ID NO:301, respectively.

In an embodiment, the GITR agonist comprises the heavy and light chain CDRs or variable regions (VRs) of 41E11. In an embodiment, the GITR agonist heavy chain variable region (V_{H}) comprises the sequence shown in SEQ ID NO:302, and the GITR agonist light chain variable region (V_{L}) comprises the sequence shown in SEQ ID NO:303, and conservative amino acid substitutions thereof. In an embodiment, a GITR agonist comprises V_{H} and V_{L} regions that are each at least 99% identical to the sequences shown in SEQ ID NO:302 and SEQ ID NO:303, respectively. In an embodiment, a GITR agonist comprises V_{H} and V_{L} regions that are each at least 98% identical to the sequences shown in SEQ ID NO:302 and SEQ ID NO:303, respectively. In an embodiment, a GITR agonist comprises V_{H} and V_{L} regions that are each at least 97% identical to the sequences shown in SEQ ID NO:302 and SEQ ID NO:303, respectively. In an embodiment, a GITR agonist comprises V_{H} and V_{L} regions that are each at least 96% identical to the sequences shown in SEQ ID NO:302 and SEQ ID NO:303, respectively. In an embodiment, a GITR agonist comprises V_{H} and V_{L} regions that are each at least 95% identical to the sequences shown in SEQ ID NO:302 and SEQ ID NO:303, respectively.

In an embodiment, a GITR agonist comprises heavy chain CDR1, CDR2 and CDR3 domains having the sequences set forth in SEQ ID NO:304, SEQ ID NO:305, and SEQ ID NO:306, respectively, and conservative amino acid substitutions thereof, and light chain CDR1, CDR2 and CDR3 domains having the sequences set forth in SEQ ID NO:307, SEQ ID NO:308, and SEQ ID NO:309, respectively, and conservative amino acid substitutions thereof.

In an embodiment, the GITR agonist is a GITR agonist biosimilar monoclonal antibody approved by drug regulatory authorities with reference to 41E11. In an embodiment, the biosimilar monoclonal antibody comprises an GITR antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 41E11. In some embodiments, the one or more post-translational modifications are selected from one or more of: glycosylation, oxidation, deamidation, and truncation. In some embodiments, the biosimilar is a GITR agonist antibody authorized or submitted for authorization, wherein the GITR agonist antibody is provided in a formulation which differs from the formulations of a reference medicinal product or reference biological product, wherein the reference medicinal product or reference biological product is 41E11. The GITR agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 41E11. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 41E11.

TABLE 28
Amino acid sequences for GITR agonist antibodies related to 41E11.

| Identi- | | |
|---|---|---|
| fier | Sequence (One-Letter Amino Acid Symbols) | |
| SEQ ID | QVQVVESGGG VVQPGRSLRL SCAASGFTFS SYGMYWVRQA PGKGLEWVAV IWYEGSNKYY | 60 |
| NO: 300 | ADSVRGRFTI SRDNSKNTLY LQMNSLRAED TALYYCARGG QLGKDYYSGM DVWGQGTTVT | 120 |
| 41E11 | VSSASTKGPS VFPLAPSSKS TSGGTAALGC LVKDYFPEPV TVSWNSGALT SGVHTFPAVL | 180 |
| heavy | QSSGLYSLSS VVTVPSSSLG TQTYICNVNH KPSNTKVDKK VEPKSCDKTH TCPPCPAPEL | 240 |
| chain | LGGPSVFLFP PKPKDTLMIS RTPEVTCVVV DVSHEDPEVK FNWYVDGVEV HNAKTKPREE | 300 |
| | QYNSTYRVVS VLTVLHQDWL NGKEYKCKVS NKALPAPIEK TISKAKGQPR EPQVYTLPPS | 360 |
| | REEMTKNQVS LTCLVKGFYP SDIAVEWESN GQPENNYKTT PPVLDSDGSF FLYSKLTVDK | 420 |
| | SRWQQGNVFS CSVMHEALHN HYTQKSLSLS PGK | 453 |
| SEQ ID | DIQMTQSPSS LSASVGDRVT ITCRASQVIR NDLGWYQQKP GKAPKRLIYA ASSLQSGVPS | 60 |
| NO: 301 | RFSGSGSGTE FTLTISSLQP EDFATYYCLQ HNSYPLTFGG GTKVEIKRTV AAPSVFIFPP | 120 |
| 41E11 | SDEQLKSGTA SVVCLLNNFY PREAKVQWKV DNALQSGNSQ ESVTEQDSKD STYSLSSTLT | 180 |
| light | LSKADYEKHK VYACEVTHQG LSSPVTKSFN RGEC | 214 |
| chain | | |
| SEQ ID | QVQVVESGGG VVQPGRSLRL SCAASGFTFS SYGMYWVRQA PGKGLEWVAV IWYEGSNKYY | 60 |
| NO: 302 | ADSVRGRFTI SRDNSKNTLY LQMNSLRAED TALYYCARGG QLGKDYYSGM DVWGQGTTVT | 120 |
| 41E11 | VSS | 123 |
| variable | | |
| heavy | | |
| chain | | |
| SEQ ID | DIQMTQSPSS LSASVGDRVT ITCRASQVIR NDLGWYQQKP GKAPKRLIYA ASSLQSGVPS | 60 |
| NO: 303 | RFSGSGSGTE FTLTISSLQP EDFATYYCLQ HNSYPLTFGG GTKVEIKR | 108 |
| 41E11 | | |
| variable | | |
| light | | |
| chain | | |
| SEQ ID | SYGMY | 5 |
| NO: 304 | | |
| 41E11 | | |
| heavy | | |
| chain | | |
| CDR1 | | |
| SEQ ID | VIWYEGSNKY YADSVRG | 17 |
| NO: 305 | | |
| 41E11 | | |
| heavy | | |
| chain | | |
| CDR2 | | |
| SEQ ID | GGQLGKDYYS GMDV | 14 |
| NO: 306 | | |
| 41E11 | | |
| heavy | | |
| chain | | |
| CDR3 | | |
| SEQ ID | RASQVIRNDL G | 11 |
| NO: 307 | | |
| 41E11 | | |
| light | | |
| chain | | |
| CDR1 | | |
| SEQ ID | AASSLQS | 7 |
| NO: 308 | | |
| 41E11 | | |
| light | | |
| chain | | |
| CDR2 | | |
| SEQ ID | LQHNSYPLT | 9 |
| NO: 309 | | |
| 41E11 | | |
| light | | |
| chain | | |
| CDR3 | | |

In a preferred embodiment, the GITR agonist is the monoclonal antibody 41G5, or a fragment, derivative, variant, or biosimilar thereof. 41G5 is available from Amgen, Inc. The preparation and properties of 41G5 are described in U.S. Patent Application Publication No. US 2015/0064204 A1, the disclosures of which are incorporated by reference herein. The amino acid sequences of 41G5 are set forth in Table 29.

In an embodiment, a GITR agonist comprises a heavy chain given by SEQ ID NO:310 and a light chain given by SEQ TD NO:311. In an embodiment, a GITR agonist comprises heavy and light chains having the sequences shown in SEQ ID NO:310 and SEQ TD NO:311, respectively, or antigen binding fragments, Fab fragments, single-chain variable fragments (scFv), variants, or conjugates thereof. In an embodiment, a GITR agonist comprises heavy and light chains that are each at least 9900 identical to the sequences shown in SEQ ID NO:310 and SEQ ID NO:311, respectively. In an embodiment, a GITR agonist comprises heavy and light chains that are each at least 98% identical to the sequences shown in SEQ ID NO:310 and SEQ ID NO:311, respectively. In an embodiment, a GITR agonist comprises heavy and light chains that are each at least 97% identical to the sequences shown in SEQ ID NO:310 and SEQ ID NO:311, respectively. In an embodiment, a GITR agonist comprises heavy and light chains that are each at least 96% identical to the sequences shown in SEQ ID NO:310 and SEQ ID NO:311, respectively. In an embodiment, a GITR agonist comprises heavy and light chains that are each at least 95% identical to the sequences shown in SEQ ID NO:310 and SEQ ID NO:311, respectively.

In an embodiment, the GITR agonist comprises the heavy and light chain CDRs or variable regions (VRs) of 41G5. In an embodiment, the GITR agonist heavy chain variable region (V_{H}) comprises the sequence shown in SEQ ID NO:312, and the GITR agonist light chain variable region (V_{L}) comprises the sequence shown in SEQ ID NO:313, and conservative amino acid substitutions thereof. In an embodiment, a GITR agonist comprises V_{H} and V_{L} regions that are each at least 99% identical to the sequences shown in SEQ ID NO:312 and SEQ ID NO:313, respectively. In an embodiment, a GITR agonist comprises V_{H} and V_{L} regions that are each at least 98% identical to the sequences shown in SEQ ID NO:312 and SEQ ID NO:313, respectively. In an embodiment, a GITR agonist comprises V_{H} and V_{L} regions that are each at least 97% identical to the sequences shown in SEQ ID NO:312 and SEQ ID NO:313, respectively. In an embodiment, a GITR agonist comprises V_{H} and V_{L} regions that are each at least 96% identical to the sequences shown in SEQ ID NO:312 and SEQ ID NO:313, respectively. In an embodiment, a GITR agonist comprises V_{H} and V_{L} regions that are each at least 95% identical to the sequences shown in SEQ ID NO:312 and SEQ ID NO:313, respectively.

In an embodiment, a GITR agonist comprises heavy chain CDR1, CDR2 and CDR3 domains having the sequences set forth in SEQ ID NO:314, SEQ ID NO:315, and SEQ ID NO:316, respectively, and conservative amino acid substitutions thereof, and light chain CDR1, CDR2 and CDR3 domains having the sequences set forth in SEQ ID NO:317, SEQ ID NO:318, and SEQ ID NO:319, respectively, and conservative amino acid substitutions thereof.

In an embodiment, the GITR agonist is a GITR agonist biosimilar monoclonal antibody approved by drug regulatory authorities with reference to 41G5. In an embodiment, the biosimilar monoclonal antibody comprises an GITR antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 41G5. In some embodiments, the one or more post-translational modifications are selected from one or more of: glycosylation, oxidation, deamidation, and truncation. In some embodiments, the biosimilar is a GITR agonist antibody authorized or submitted for authorization, wherein the GITR agonist antibody is provided in a formulation which differs from the formulations of a reference medicinal product or reference biological product, wherein the reference medicinal product or reference biological product is 41G5. The GITR agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 41G5. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 41G5.

TABLE 29
Amino acid sequences for GITR agonist antibodies related to 41G5.

| Identi- | | |
|---|---|---|
| fier | Sequence (One-Letter Amino Acid Symbols) | |
| SEQ ID | QVQLVESGGG VVQPGRSLRL SCAASGFTFS SYGMHWVRQA PGKGLEWVAV IWYPGSNKYY | 60 |
| NO: 310 | ADSVKGRFTI SRDNSKNTLY LQMNSLRAED TAVYYCARGG ELGRYYYYGM DVWGQGTTVT | 120 |
| 41G5 | VSSASTKGPS VFPLAPSSKS TSGGTAALGC LVKDYFPEPV TVSWNSGALT SGVHTFPAVL | 180 |
| heavy | QSSGLYSLSS VVTVPSSSLG TQTYICNVNH KPSNTKVDKK VEPKSCDKTH TCPPCPAPEL | 240 |
| chain | LGGPSVFLFP PKPKDTLMIS RTPEVTCVVV DVSHEDPEVK FNWYVDGVEV HNAKTKPREE | 300 |
| | QYNSTYRVVS VLTVLHQDWL NGKEYKCKVS NKALPAPIEK TISKAKGQPR EPQVYTLPPS | 360 |
| | REEMTKNQVS LTCLVKGFYP SDIAVEWESN GQPENNYKTT PPVLDSDGSF FLYSKLTVDK | 420 |
| | SRWQQGNVFS CSVMHEALHN HYTQKSLSLS PGK | 453 |
| SEQ ID | DIQMTQSPSS LSASVGDRVT VTCRASQGIR NDLGWYQQKP GKAPKRLIYA ASSLQSGVPS | 60 |
| NO: 311 | RFSGSGSGTE FTLTISSLQP EDFATYYCLQ HNNYPWTFGQ GTKVDIKRTV AAPSVFIFPP | 120 |
| 41G5 | SDEQLKSGTA SVVCLLNNFY PREAKVQWKV DNALQSGNSQ ESVTEQDSKD STYSLSSTLT | 180 |
| light | LSKADYEKHK VYACEVTHQG LSSPVTKSFN RGEC | 214 |
| chain | | |
| SEQ ID | QVQLVESGGG VVQPGRSLRL SCAASGFTFS SYGMHWVRQA PGKGLEWVAV IWYPGSNKYY | 60 |
| NO: 312 | ADSVKGRFTI SRDNSKNTLY LQMNSLRAED TAVYYCARGG ELGRYYYYGM DVWGQGTTVT | 120 |
| 41G5 | VSS | 123 |
| variable | | |
| heavy | | |
| chain | | |
| SEQ ID | DIQMTQSPSS LSASVGDRVT VTCRASQGIR NDLGWYQQKP GKAPKRLIYA ASSLQSGVPS | 60 |
| NO: 313 | RFSGSGSGTE FTLTISSLQP EDFATYYCLQ HNNYPWTFGQ GTKVDIKR | 108 |
| 41G5 | | |
| variable | | |
| light | | |
| chain | | |
| SEQ ID | SYGMH | 5 |
| NO: 314 | | |
| 41G5 | | |
| heavy | | |
| chain | | |
| CDR1 | | |
| SEQ ID | VIWYPGSNKY YADSVKG | 17 |
| NO: 315 | | |
| 41G5 | | |
| heavy | | |
| chain | | |
| CDR2 | | |
| SEQ ID | GGELGRYYYY GMDV | 14 |
| NO: 316 | | |
| 41G5 | | |
| heavy | | |
| chain | | |
| CDR3 | | |
| SEQ ID | RASQGIRNDL G | 11 |
| NO: 317 | | |
| 41G5 | | |
| light | | |
| chain | | |
| CDR1 | | |
| SEQ ID | AASSLQS | 7 |
| NO: 318 | | |
| 41G5 | | |
| light | | |
| chain | | |
| CDR2 | | |
| SEQ ID | LQHNNYPWT | 9 |
| NO: 319 | | |
| 41G5 | | |
| light | | |
| chain | | |
| CDR3 | | |

In a preferred embodiment, the GITR agonist is the monoclonal antibody 42A11, or a fragment, derivative, variant, or biosimilar thereof 42A11 is available from Amgen, Inc. The preparation and properties of 42A11 are described in U.S. Patent Application Publication No. US 2015/0064204 A1, the disclosures of which are incorporated by reference herein. The amino acid sequences of 42A11 are set forth in Table 30.

In an embodiment, a GITR agonist comprises a heavy chain given by SEQ ID NO:320 and a light chain given by SEQ ID NO:321. In an embodiment, a GITR agonist comprises heavy and light chains having the sequences shown in SEQ ID NO:320 and SEQ ID NO:321, respectively, or antigen binding fragments, Fab fragments, single-chain variable fragments (scFv), variants, or conjugates thereof. In an embodiment, a GITR agonist comprises heavy and light chains that are each at least 99% identical to the sequences shown in SEQ ID NO:320 and SEQ ID NO:321, respectively. In an embodiment, a GITR agonist comprises heavy and light chains that are each at least 98% identical to the sequences shown in SEQ ID NO:320 and SEQ ID NO:321, respectively. In an embodiment, a GITR agonist comprises heavy and light chains that are each at least 97% identical to the sequences shown in SEQ ID NO:320 and SEQ ID NO:321, respectively. In an embodiment, a GITR agonist comprises heavy and light chains that are each at least 96% identical to the sequences shown in SEQ ID NO:320 and SEQ ID NO:321, respectively. In an embodiment, a GITR agonist comprises heavy and light chains that are each at least 95% identical to the sequences shown in SEQ ID NO:320 and SEQ ID NO:321, respectively.

In an embodiment, the GITR agonist comprises the heavy and light chain CDRs or variable regions (VRs) of 42A11. In an embodiment, the GITR agonist heavy chain variable region (V_{H}) comprises the sequence shown in SEQ ID NO:322, and the GITR agonist light chain variable region (V_{L}) comprises the sequence shown in SEQ ID NO:323, and conservative amino acid substitutions thereof. In an embodiment, a GITR agonist comprises V_{H} and V_{L} regions that are each at least 99% identical to the sequences shown in SEQ ID NO:322 and SEQ ID NO:323, respectively. In an embodiment, a GITR agonist comprises V_{H} and V_{L} regions that are each at least 98% identical to the sequences shown in SEQ ID NO:322 and SEQ ID NO:323, respectively. In an embodiment, a GITR agonist comprises V_{H} and V_{L} regions that are each at least 97% identical to the sequences shown in SEQ ID NO:322 and SEQ ID NO:323, respectively. In an embodiment, a GITR agonist comprises V_{H} and V_{L} regions that are each at least 96% identical to the sequences shown in SEQ ID NO:322 and SEQ ID NO:323, respectively. In an embodiment, a GITR agonist comprises V_{H} and V_{L} regions that are each at least 95% identical to the sequences shown in SEQ ID NO:322 and SEQ ID NO:323, respectively.

In an embodiment, a GITR agonist comprises heavy chain CDR1, CDR2 and CDR3 domains having the sequences set forth in SEQ ID NO:324, SEQ ID NO:325, and SEQ ID NO:326, respectively, and conservative amino acid substitutions thereof, and light chain CDR1, CDR2 and CDR3 domains having the sequences set forth in SEQ ID NO:327, SEQ ID NO:328, and SEQ ID NO:329, respectively, and conservative amino acid substitutions thereof.

In an embodiment, the GITR agonist is a GITR agonist biosimilar monoclonal antibody approved by drug regulatory authorities with reference to 42A11. In an embodiment, the biosimilar monoclonal antibody comprises an GITR antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 42A11. In some embodiments, the one or more post-translational modifications are selected from one or more of: glycosylation, oxidation, deamidation, and truncation. In some embodiments, the biosimilar is a GITR agonist antibody authorized or submitted for authorization, wherein the GITR agonist antibody is provided in a formulation which differs from the formulations of a reference medicinal product or reference biological product, wherein the reference medicinal product or reference biological product is 42A11. The GITR agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 42A11. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 42A11.

TABLE 30
Amino acid sequences for GITR agonist antibodies related to 42A11.

| Identi- | | |
|---|---|---|
| fier | Sequence (One-Letter Amino Acid Symbols) | |
| SEQ ID | QVQLVESGGG VVQPGRSLRL SCAASGFTFS SYGMHWVRQA PGKGLEWVAV IWYEGSNKYY | 60 |
| NO: 320 | ADSVKGRFTI SRDNSKNTLY LQMNSLRAED TAVYYCARGG QLGYYYYSGM DVWGQGTTVT | 120 |
| 42A11 | VSSASTKGPS VFPLAPSSKS TSGGTAALGC LVKDYFPEPV TVSWNSGALT SGVHTFPAVL | 180 |
| heavy | QSSGLYSLSS VVTVPSSSLG TQTYICNVNH KPSNTKVDKK VEPKSCDKTH TCPPCPAPEL | 240 |
| chain | LGGPSVFLFP PKPKDTLMIS RTPEVTCVVV DVSHEDPEVK FNWYVDGVEV HNAKTKPREE | 300 |
| | QYNSTYRVVS VLTVLHQDWL NGKEYKCKVS NKALPAPIEK TISKAKGQPR EPQVYTLPPS | 360 |
| | REEMTKNQVS LTCLVKGFYP SDIAVEWESN GQPENNYKTT PPVLDSDGSF FLYSKLTVDK | 420 |
| | SRWQQGNVFS CSVMHEALHN HYTQKSLSLS PGK | 453 |
| SEQ ID | DIQMTQSPSS LSASVGDRVT ITCRASQGIR NDLGWYQQKP GKAPKRLIYD ASSLQSGVPS | 60 |
| NO: 321 | RFSGSGSGTD FTLTISSLQP EEFATYYCLQ HNNYPWTFGQ GTKVEIKRTV AAPSVFIFPP | 120 |
| 42A11 | SDEQLKSGTA SVVCLLNNFY PREAKVQWKV DNALQSGNSQ ESVTEQDSKD STYSLSSTLT | 180 |
| light | LSKADYEKHK VYACEVTHQG LSSPVTKSFN RGEC | 214 |
| chain | | |
| SEQ ID | QVQLVESGGG VVQPGRSLRL SCAASGFTFS SYGMHWVRQA PGKGLEWVAV IWYEGSNKYY | 60 |
| NO: 322 | ADSVKGRFTI SRDNSKNTLY LQMNSLRAED TAVYYCARGG QLGYYYYSGM DVWGQGTTVT | 120 |
| 42A11 | VSS | 123 |
| variable | | |
| heavy | | |
| chain | | |
| SEQ ID | DIQMTQSPSS LSASVGDRVT ITCRASQGIR NDLGWYQQKP GKAPKRLIYD ASSLQSGVPS | 60 |
| NO: 323 | RFSGSGSGTD FTLTISSLQP EEFATYYCLQ HNNYPWTFGQ GTKVEIKR | 108 |
| 42A11 | | |
| variable | | |
| light | | |
| chain | | |
| SEQ ID | SYGMH | 5 |
| NO: 324 | | |
| 42A11 | | |
| heavy | | |
| chain | | |
| CDR1 | | |
| SEQ ID | VIWYEGSNKY YADSVKG | 17 |
| NO: 325 | | |
| 42A11 | | |
| heavy | | |
| chain | | |
| CDR2 | | |
| SEQ ID | GGQLGYYYYS GMDV | 14 |
| NO: 326 | | |
| 42A11 | | |
| heavy | | |
| chain | | |
| CDR3 | | |
| SEQ ID | RASQGIRNDL G | 11 |
| NO: 327 | | |
| 42A11 | | |
| light | | |
| chain | | |
| CDR1 | | |
| SEQ ID | DASSLQS | 7 |
| NO: 328 | | |
| 42A11 | | |
| light | | |
| chain | | |
| CDR2 | | |
| SEQ ID | LQHNNYPWT | 9 |
| NO: 329 | | |
| 42A11 | | |
| light | | |
| chain | | |
| CDR3 | | |

In a preferred embodiment, the GITR agonist is the monoclonal antibody 44C1, or a fragment, derivative, variant, or biosimilar thereof. 44C1 is available from Amgen, Inc. The preparation and properties of 44C1 are described in U.S. Patent Application Publication No. US 2015/0064204 A1, the disclosures of which are incorporated by reference herein. The amino acid sequences of 44C1 are set forth in Table 31.

In an embodiment, a GITR agonist comprises a heavy chain given by SEQ ID NO:330 and a light chain given by SEQ ID NO:331. In an embodiment, a GITR agonist comprises heavy and light chains having the sequences shown in SEQ ID NO:330 and SEQ ID NO:331, respectively, or antigen binding fragments, Fab fragments, single-chain variable fragments (scFv), variants, or conjugates thereof. In an embodiment, a GITR agonist comprises heavy and light chains that are each at least 99% identical to the sequences shown in SEQ ID NO:330 and SEQ ID NO:331, respectively. In an embodiment, a GITR agonist comprises heavy and light chains that are each at least 98% identical to the sequences shown in SEQ ID NO:330 and SEQ ID NO:331, respectively. In an embodiment, a GITR agonist comprises heavy and light chains that are each at least 97% identical to the sequences shown in SEQ ID NO:330 and SEQ ID NO:331, respectively. In an embodiment, a GITR agonist comprises heavy and light chains that are each at least 96% identical to the sequences shown in SEQ ID NO:330 and SEQ ID NO:331, respectively. In an embodiment, a GITR agonist comprises heavy and light chains that are each at least 95% identical to the sequences shown in SEQ ID NO:330 and SEQ ID NO:331, respectively.

In an embodiment, the GITR agonist comprises the heavy and light chain CDRs or variable regions (VRs) of 44C1. In an embodiment, the GITR agonist heavy chain variable region (V_{H}) comprises the sequence shown in SEQ ID NO:332, and the GITR agonist light chain variable region (V_{L}) comprises the sequence shown in SEQ ID NO:333, and conservative amino acid substitutions thereof. In an embodiment, a GITR agonist comprises V_{H} and V_{L} regions that are each at least 99% identical to the sequences shown in SEQ ID NO:332 and SEQ ID NO:333, respectively. In an embodiment, a GITR agonist comprises V_{H} and V_{L} regions that are each at least 98% identical to the sequences shown in SEQ ID NO:332 and SEQ ID NO:333, respectively. In an embodiment, a GITR agonist comprises V_{H} and V_{L} regions that are each at least 97% identical to the sequences shown in SEQ ID NO:332 and SEQ ID NO:333, respectively. In an embodiment, a GITR agonist comprises V_{H} and V_{L} regions that are each at least 96% identical to the sequences shown in SEQ ID NO:332 and SEQ ID NO:333, respectively. In an embodiment, a GITR agonist comprises V_{H} and V_{L} regions that are each at least 95% identical to the sequences shown in SEQ ID NO:332 and SEQ ID NO:333, respectively.

In an embodiment, a GITR agonist comprises heavy chain CDR1, CDR2 and CDR3 domains having the sequences set forth in SEQ ID NO:334, SEQ ID NO:335, and SEQ ID NO:336, respectively, and conservative amino acid substitutions thereof, and light chain CDR1, CDR2 and CDR3 domains having the sequences set forth in SEQ ID NO:337, SEQ ID NO:338, and SEQ ID NO:339, respectively, and conservative amino acid substitutions thereof.

In an embodiment, the GITR agonist is a GITR agonist biosimilar monoclonal antibody approved by drug regulatory authorities with reference to 44C1. In an embodiment, the biosimilar monoclonal antibody comprises an GITR antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 44C1. In some embodiments, the one or more post-translational modifications are selected from one or more of: glycosylation, oxidation, deamidation, and truncation. In some embodiments, the biosimilar is a GITR agonist antibody authorized or submitted for authorization, wherein the GITR agonist antibody is provided in a formulation which differs from the formulations of a reference medicinal product or reference biological product, wherein the reference medicinal product or reference biological product is 44C1. The GITR agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 44C1. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 44C1.

TABLE 31
Amino acid sequences for GITR agonist antibodies related to 44C1.

| Identi- | | |
|---|---|---|
| fier | Sequence (One-Letter Amino Acid Symbols) | |
| SEQ ID | QVQLVESGGG VVQPGRSLRL SCAASGFTLS SYGMHWVRQA PGKGLEWVAV IWYDGSNKYY | 60 |
| NO: 330 | ADSVKGRFTI SRDNSKNTLY LQMNSLRAED TAVYYCARRG TVTTPDFDYW GQGTLVTVSS | 120 |
| 44C1 | ASTKGPSVFP LAPSSKSTSG GTAALGCLVK DYFPEPVTVS WNSGALTSGV HTFPAVLQSS | 180 |
| heavy | GLYSLSSVVT VPSSSLGTQT YICNVNHKPS NTKVDKKVEP KSCDKTHTCP PCPAPELLGG | 240 |
| chain | PSVFLFPPKP KDTLMISRTP EVTCVVVDVS HEDPEVKFNW YVDGVEVHNA KTKPREEQYN | 300 |
| | STYRVVSVLT VLHQDWLNGK EYKCKVSNKA LPAPIEKTIS KAKGQPREPQ VYTLPPSREE | 360 |
| | MTKNQVSLTC LVKGFYPSDI AVEWESNGQP ENNYKTTPPV LDSDGSFFLY SKLTVDKSRW | 420 |
| | QQGNVFSCSV MHEALHNHYT QKSLSLSPGK | 450 |
| SEQ ID | QSALTQPASV SGSPGQSITI SCTGTSSDVG TYNLVSWYQQ HPGKAPKLMI YEVSKRPSGV | 60 |
| NO: 331 | SNRFSGSKSG NTASLTISGL QAEDEADYYC CSYAGFSTWV FGGGTKLTVL GQPKAAPSVT | 120 |
| 44C1 | LFPPSSEELQ ANKATLVCLI SDFYPGAVTV AWKADSSPVK AGVETTTPSK QSNNKYAASS | 180 |
| light | YLSLTPEQWK SHRSYSCQVT HEGSTVEKTV APTECS | 216 |
| chain | | |
| SEQ ID | QVQLVESGGG VVQPGRSLRL SCAASGFTLS SYGMHWVRQA PGKGLEWVAV IWYDGSNKYY | 60 |
| NO: 332 | ADSVKGRFTI SRDNSKNTLY LQMNSLRAED TAVYYCARRG TVTTPDFDYW GQGTLVTVSS | 120 |
| 44C1 | | |
| variable | | |
| heavy | | |
| chain | | |
| SEQ ID | QSALTQPASV SGSPGQSITI SCTGTSSDVG TYNLVSWYQQ HPGKAPKLMI YEVSKRPSGV | 60 |
| NO: 333 | SNRFSGSKSG NTASLTISGL QAEDEADYYC CSYAGFSTWV FGGGTKLTVL G | 111 |
| 44C1 | | |
| variable | | |
| light | | |
| chain | | |
| SEQ ID | SYGMH | 5 |
| NO: 334 | | |
| 44C1 | | |
| heavy | | |
| chain | | |
| CDR1 | | |
| SEQ ID | VIWYDGSNKY YADSVKG | 17 |
| NO: 335 | | |
| 44C1 | | |
| heavy | | |
| chain | | |
| CDR2 | | |
| SEQ ID | RGTVTTPDFD Y | 11 |
| NO: 336 | | |
| 44C1 | | |
| heavy | | |
| chain | | |
| CDR3 | | |
| SEQ ID | TGTSSDVGTY NLVS | 14 |
| NO: 337 | | |
| 44C1 | | |
| light | | |
| chain | | |
| CDR1 | | |
| SEQ ID | EVSKRPS | 7 |
| NO: 338 | | |
| 44C1 | | |
| light | | |
| chain | | |
| CDR2 | | |
| SEQ ID | CSYAGFSTWV | 10 |
| NO: 339 | | |
| 44C1 | | |
| light | | |
| chain | | |
| CDR3 | | |

In a preferred embodiment, the GITR agonist is the monoclonal antibody 45A8, or a fragment, derivative, variant, or biosimilar thereof. 45A8 is available from Amgen, Inc. The preparation and properties of 45A8 are described in U.S. Patent Application Publication No. US 2015/0064204 A1, the disclosures of which are incorporated by reference herein. The amino acid sequences of 45A8 are set forth in Table 32.

In an embodiment, a GITR agonist comprises a heavy chain given by SEQ ID NO:340 and a light chain given by SEQ TD NO:341. In an embodiment, a GITR agonist comprises heavy and light chains having the sequences shown in SEQ ID NO:340 and SEQ ID NO:341, respectively, or antigen binding fragments, Fab fragments, single-chain variable fragments (scFv), variants, or conjugates thereof. In an embodiment, a GITR agonist comprises heavy and light chains that are each at least 99% identical to the sequences shown in SEQ ID NO:340 and SEQ ID NO:341, respectively. In an embodiment, a GITR agonist comprises heavy and light chains that are each at least 98% identical to the sequences shown in SEQ ID NO:340 and SEQ ID NO:341, respectively. In an embodiment, a GITR agonist comprises heavy and light chains that are each at least 97% identical to the sequences shown in SEQ ID NO:340 and SEQ ID NO:341, respectively. In an embodiment, a GITR agonist comprises heavy and light chains that are each at least 96% identical to the sequences shown in SEQ ID NO:340 and SEQ ID NO:341, respectively. In an embodiment, a GITR agonist comprises heavy and light chains that are each at least 95% identical to the sequences shown in SEQ ID NO:340 and SEQ ID NO:341, respectively.

In an embodiment, the GITR agonist comprises the heavy and light chain CDRs or variable regions (VRs) of 45A8. In an embodiment, the GITR agonist heavy chain variable region (V_{H}) comprises the sequence shown in SEQ ID NO:342, and the GITR agonist light chain variable region (V_{L}) comprises the sequence shown in SEQ ID NO:343, and conservative amino acid substitutions thereof. In an embodiment, a GITR agonist comprises V_{H} and V_{L} regions that are each at least 99% identical to the sequences shown in SEQ ID NO:342 and SEQ ID NO:343, respectively. In an embodiment, a GITR agonist comprises V_{H} and V_{L} regions that are each at least 98% identical to the sequences shown in SEQ ID NO:342 and SEQ ID NO:343, respectively. In an embodiment, a GITR agonist comprises V_{H} and V_{L} regions that are each at least 97% identical to the sequences shown in SEQ ID NO:342 and SEQ ID NO:343, respectively. In an embodiment, a GITR agonist comprises V_{H} and V_{L} regions that are each at least 96% identical to the sequences shown in SEQ ID NO:342 and SEQ ID NO:343, respectively. In an embodiment, a GITR agonist comprises V_{H} and V_{L} regions that are each at least 95% identical to the sequences shown in SEQ ID NO:342 and SEQ ID NO:343, respectively.

In an embodiment, a GITR agonist comprises heavy chain CDR1, CDR2 and CDR3 domains having the sequences set forth in SEQ ID NO:344, SEQ ID NO:345, and SEQ ID NO:346, respectively, and conservative amino acid substitutions thereof, and light chain CDR1, CDR2 and CDR3 domains having the sequences set forth in SEQ ID NO:347, SEQ ID NO:348, and SEQ ID NO:349, respectively, and conservative amino acid substitutions thereof.

In an embodiment, the GITR agonist is a GITR agonist biosimilar monoclonal antibody approved by drug regulatory authorities with reference to 45A8. In an embodiment, the biosimilar monoclonal antibody comprises an GITR antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 45A8. In some embodiments, the one or more post-translational modifications are selected from one or more of: glycosylation, oxidation, deamidation, and truncation. In some embodiments, the biosimilar is a GITR agonist antibody authorized or submitted for authorization, wherein the GITR agonist antibody is provided in a formulation which differs from the formulations of a reference medicinal product or reference biological product, wherein the reference medicinal product or reference biological product is 45A8. The GITR agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 45A8. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 45A8.

TABLE 32
Amino acid sequences for GITR agonist antibodies related to 45A8.

| Identi- | | |
|---|---|---|
| fier | Sequence (One-Letter Amino Acid Symbols) | |
| SEQ ID | QVQLVESGGG VVQPGRSLRL SCAASGFTFS SYGMHWVRQA PGKGLEWVAV IWHDGSNKYY | 60 |
| NO: 340 | ADSVKGRFTI SKDNSKNTLY LQMNSLRAED TAVYYCAREY GGNFDYWGQG TLVTVSSAST | 120 |
| 45A8 | KGPSVFPLAP SSKSTSGGTA ALGCLVKDYF PEPVTVSWNS GALTSGVHTF PAVLQSSGLY | 180 |
| heavy | SLSSVVTVPS SSLGTQTYIC NVNHKPSNTK VDKKVEPKSC DKTHTCPPCP APELLGGPSV | 240 |
| chain | FLFPPKPKDT LMISRTPEVT CVVVDVSHED PEVKFNWYVD GVEVHNAKTK PREEQYNSTY | 300 |
| | RVVSVLTVLH QDWLNGKEYK CKVSNKALPA PIEKTISKAK GQPREPQVYT LPPSREEMTK | 360 |
| | NQVSLTCLVK GFYPSDIAVE WESNGQPENN YKTTPPVLDS DGSFFLYSKL TVDKSRWQQG | 420 |
| | NVFSCSVMHE ALHNHYTQKS LSLSPGK | 447 |
| SEQ ID | QSALTQPASV SGSPGQSITI SCTGTSSDVG TYNLVSWYQQ HPGKAPKLMI YEVSKRPSGI | 60 |
| NO: 341 | SNRFSGSKSG NTASLTISGL QAEDEADYYC CSYAGYSTWV FGGGTKLTVL RQPKAAPSVT | 120 |
| 45A8 | LFPPSSEELQ ANKATLVCLI SDFYPGAVTV AWKADSSPVK AGVETTTPSK QSNNKYAASS | 180 |
| light | YLSLTPEQWK SHRSYSCQVT HEGSTVEKTV APTECS | 216 |
| chain | | |
| SEQ ID | QVQLVESGGG VVQPGRSLRL SCAASGFTFS SYGMHWVRQA PGKGLEWVAV IWHDGSNKYY | 60 |
| NO: 342 | ADSVKGRFTI SKDNSKNTLY LQMNSLRAED TAVYYCAREY GGNFDYWGQG TLVTVSS | 117 |
| 45A8 | | |
| variable | | |
| heavy | | |
| chain | | |
| SEQ ID | QSALTQPASV SGSPGQSITI SCTGTSSDVG TYNLVSWYQQ HPGKAPKLMI YEVSKRPSGI | 60 |
| NO: 343 | SNRFSGSKSG NTASLTISGL QAEDEADYYC CSYAGYSTWV FGGGTKLTVL R | 111 |
| 45A8 | | |
| variable | | |
| light | | |
| chain | | |
| SEQ ID | SYGMH | 5 |
| NO: 344 | | |
| 45A8 | | |
| heavy | | |
| chain | | |
| CDR1 | | |
| SEQ ID | VIWHDGSNKY YADSVKG | 17 |
| NO: 345 | | |
| 45A8 | | |
| heavy | | |
| chain | | |
| CDR2 | | |
| SEQ ID | EYGGNFDY | 8 |
| NO: 346 | | |
| 45A8 | | |
| heavy | | |
| chain | | |
| CDR3 | | |
| SEQ ID | TGTSSDVGTY NLVS | 14 |
| NO: 347 | | |
| 45A8 | | |
| light | | |
| chain | | |
| CDR1 | | |
| SEQ ID | EVSKRPS | 7 |
| NO: 348 | | |
| 45A8 | | |
| light | | |
| chain | | |
| CDR2 | | |
| SEQ ID | CSYAGYSTWV | 10 |
| NO: 349 | | |
| 45A8 | | |
| light | | |
| chain | | |
| CDR3 | | |

In a preferred embodiment, the GITR agonist is the monoclonal antibody 46E11, or a fragment, derivative, variant, or biosimilar thereof. 46E11 is available from Amgen, Inc. The preparation and properties of 46E11 are described in U.S. Patent Application Publication No. US 2015/0064204 A1, the disclosures of which are incorporated by reference herein. The amino acid sequences of 46E11 are set forth in Table 33.

In an embodiment, a GITR agonist comprises a heavy chain given by SEQ ID NO:350 and a light chain given by SEQ ID NO:351. In an embodiment, a GITR agonist comprises heavy and light chains having the sequences shown in SEQ ID NO:350 and SEQ ID NO:351, respectively, or antigen binding fragments, Fab fragments, single-chain variable fragments (scFv), variants, or conjugates thereof. In an embodiment, a GITR agonist comprises heavy and light chains that are each at least 99% identical to the sequences shown in SEQ ID NO:350 and SEQ ID NO:351, respectively. In an embodiment, a GITR agonist comprises heavy and light chains that are each at least 98% identical to the sequences shown in SEQ ID NO:350 and SEQ ID NO:351, respectively. In an embodiment, a GITR agonist comprises heavy and light chains that are each at least 97% identical to the sequences shown in SEQ ID NO:350 and SEQ ID NO:351, respectively. In an embodiment, a GITR agonist comprises heavy and light chains that are each at least 96% identical to the sequences shown in SEQ ID NO:350 and SEQ ID NO:351, respectively. In an embodiment, a GITR agonist comprises heavy and light chains that are each at least 95% identical to the sequences shown in SEQ ID NO:350 and SEQ ID NO:351, respectively.

In an embodiment, the GITR agonist comprises the heavy and light chain CDRs or variable regions (VRs) of 46E11. In an embodiment, the GITR agonist heavy chain variable region (V_{H}) comprises the sequence shown in SEQ ID NO:352, and the GITR agonist light chain variable region (V_{L}) comprises the sequence shown in SEQ ID NO:353, and conservative amino acid substitutions thereof. In an embodiment, a GITR agonist comprises V_{H} and V_{L} regions that are each at least 99% identical to the sequences shown in SEQ ID NO:352 and SEQ ID NO:353, respectively. In an embodiment, a GITR agonist comprises V_{H} and V_{L} regions that are each at least 98% identical to the sequences shown in SEQ ID NO:352 and SEQ ID NO:353, respectively. In an embodiment, a GITR agonist comprises V_{H} and V_{L} regions that are each at least 97% identical to the sequences shown in SEQ ID NO:352 and SEQ ID NO:353, respectively. In an embodiment, a GITR agonist comprises V_{H} and V_{L} regions that are each at least 96% identical to the sequences shown in SEQ ID NO:352 and SEQ ID NO:353, respectively. In an embodiment, a GITR agonist comprises V_{H} and V_{L} regions that are each at least 95% identical to the sequences shown in SEQ ID NO:352 and SEQ ID NO:353, respectively.

In an embodiment, a GITR agonist comprises heavy chain CDR1, CDR2 and CDR3 domains having the sequences set forth in SEQ ID NO:354, SEQ ID NO:355, and SEQ ID NO:356, respectively, and conservative amino acid substitutions thereof, and light chain CDR1, CDR2 and CDR3 domains having the sequences set forth in SEQ ID NO:357, SEQ ID NO:358, and SEQ ID NO:359, respectively, and conservative amino acid substitutions thereof.

In an embodiment, the GITR agonist is a GITR agonist biosimilar monoclonal antibody approved by drug regulatory authorities with reference to 46E11. In an embodiment, the biosimilar monoclonal antibody comprises an GITR antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 46E11. In some embodiments, the one or more post-translational modifications are selected from one or more of: glycosylation, oxidation, deamidation, and truncation. In some embodiments, the biosimilar is a GITR agonist antibody authorized or submitted for authorization, wherein the GITR agonist antibody is provided in a formulation which differs from the formulations of a reference medicinal product or reference biological product, wherein the reference medicinal product or reference biological product is 46E11. The GITR agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 46E11. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 46E11.

TABLE 33
Amino acid sequences for GITR agonist antibodies related to 46E11.

| Identi- | | |
|---|---|---|
| fier | Sequence (One-Letter Amino Acid Symbols) | |
| SEQ ID | QVQLVESGGG VVQPGRSLRL SCAASGFTFS SYGMHWVRQA PGKGLEWVAV IWYAGSNKYY | 60 |
| NO: 350 | ADSVKGRFTI SRDNSKNTLY LQMNSLRAED TAVYYCARGD ILTGYSLYYG MDVWGQGTTV | 120 |
| 46E11 | TVSSASTKGP SVFPLAPSSK STSGGTAALG CLVKDYFPEP VTVSWNSGAL TSGVHTFPAV | 180 |
| heavy | LQSSGLYSLS SVVTVPSSSL GTQTYICNVN HKPSNTKVDK KVEPKSCDKT HTCPPCPAPE | 240 |
| chain | LLGGPSVFLF PPKPKDTLMI SRTPEVTCVV VDVSHEDPEV KFNWYVDGVE VHNAKTKPRE | 300 |
| | EQYNSTYRVV SVLTVLHQDW LNGKEYKCKV SNKALPAPIE KTISKAKGQP REPQVYTLPP | 360 |
| | SREEMTKNQV SLTCLVKGFY PSDIAVEWES NGQPENNYKT TPPVLDSDGS FFLYSKLTVD | 420 |
| | KSRWQQGNVF SCSVMHEALH NHYTQKSLSL SPGK | 454 |
| SEQ ID | DIQMTQSPSS LSASVGDRVT ITCRASQGIR NDLGWYQQKP GKAPKRLIYA ASSLQSGVPS | 60 |
| NO: 351 | RFSGSGSGAE FTLTISSLQP EDFATYYCLQ HNSYPWTFGQ GTKVEIKRTV AAPSVFIFPP | 120 |
| 46E11 | SDEQLKSGTA SVVCLLNNFY PREAKVQWKV DNALQSGNSQ ESVTEQDSKD STYSLSSTLT | 180 |
| light | LSKADYEKHK VYACEVTHQG LSSPVTKSFN RGEC | 214 |
| chain | | |
| SEQ ID | QVQLVESGGG VVQPGRSLRL SCAASGFTFS SYGMHWVRQA PGKGLEWVAV IWYAGSNKYY | 60 |
| NO: 352 | ADSVKGRFTI SRDNSKNTLY LQMNSLRAED TAVYYCARGD ILTGYSLYYG MDVWGQGTTV | 120 |
| 46E11 | TVSS | 124 |
| variable | | |
| heavy | | |
| chain | | |
| SEQ ID | DIQMTQSPSS LSASVGDRVT ITCRASQGIR NDLGWYQQKP GKAPKRLIYA ASSLQSGVPS | 60 |
| NO: 353 | RFSGSGSGAE FTLTISSLQP EDFATYYCLQ HNSYPWTFGQ GTKVEIKR | 108 |
| 46E11 | | |
| variable | | |
| light | | |
| chain | | |
| SEQ ID | SYGMH | 5 |
| NO: 354 | | |
| 46E11 | | |
| heavy | | |
| chain | | |
| CDR1 | | |
| SEQ ID | VIWYAGSNKY YADSVKG | 17 |
| NO: 355 | | |
| 46E11 | | |
| heavy | | |
| chain | | |
| CDR2 | | |
| SEQ ID | GDILTGYSLY YGMDV | 15 |
| NO: 356 | | |
| 46E11 | | |
| heavy | | |
| chain | | |
| CDR3 | | |
| SEQ ID | RASQGIRNDL G | 11 |
| NO: 357 | | |
| 46E11 | | |
| light | | |
| chain | | |
| CDR1 | | |
| SEQ ID | AASSLQS | 7 |
| NO: 358 | | |
| 46E11 | | |
| light | | |
| chain | | |
| CDR2 | | |
| SEQ ID | LQHNSYPWT | 9 |
| NO: 359 | | |
| 46E11 | | |
| light | | |
| chain | | |
| CDR3 | | |

In a preferred embodiment, the GITR agonist is the monoclonal antibody 48H12, or a fragment, derivative, variant, or biosimilar thereof 48H12 is available from Amgen, Inc. The preparation and properties of 48H12 are described in U.S. Patent Application Publication No. US 2015/0064204 A1, the disclosures of which are incorporated by reference herein. The amino acid sequences of 48H12 are set forth in Table 34.

In an embodiment, a GITR agonist comprises a heavy chain given by SEQ ID NO:360 and a light chain given by SEQ ID NO:361. In an embodiment, a GITR agonist comprises heavy and light chains having the sequences shown in SEQ ID NO:360 and SEQ ID NO:361, respectively, or antigen binding fragments, Fab fragments, single-chain variable fragments (scFv), variants, or conjugates thereof. In an embodiment, a GITR agonist comprises heavy and light chains that are each at least 99% identical to the sequences shown in SEQ ID NO:360 and SEQ ID NO:361, respectively. In an embodiment, a GITR agonist comprises heavy and light chains that are each at least 98% identical to the sequences shown in SEQ ID NO:360 and SEQ ID NO:361, respectively. In an embodiment, a GITR agonist comprises heavy and light chains that are each at least 97% identical to the sequences shown in SEQ ID NO:360 and SEQ ID NO:361, respectively. In an embodiment, a GITR agonist comprises heavy and light chains that are each at least 96% identical to the sequences shown in SEQ ID NO:360 and SEQ ID NO:361, respectively. In an embodiment, a GITR agonist comprises heavy and light chains that are each at least 95% identical to the sequences shown in SEQ ID NO:360 and SEQ ID NO:361, respectively.

In an embodiment, the GITR agonist comprises the heavy and light chain CDRs or variable regions (VRs) of 48H12. In an embodiment, the GITR agonist heavy chain variable region (V_{H}) comprises the sequence shown in SEQ ID NO:362, and the GITR agonist light chain variable region (V_{L}) comprises the sequence shown in SEQ ID NO:363, and conservative amino acid substitutions thereof. In an embodiment, a GITR agonist comprises V_{H} and V_{L} regions that are each at least 99% identical to the sequences shown in SEQ ID NO:362 and SEQ ID NO:363, respectively. In an embodiment, a GITR agonist comprises V_{H} and V_{L} regions that are each at least 98% identical to the sequences shown in SEQ ID NO:362 and SEQ ID NO:363, respectively. In an embodiment, a GITR agonist comprises V_{H} and V_{L} regions that are each at least 97% identical to the sequences shown in SEQ ID NO:362 and SEQ ID NO:363, respectively. In an embodiment, a GITR agonist comprises V_{H} and V_{L} regions that are each at least 96% identical to the sequences shown in SEQ ID NO:362 and SEQ ID NO:363, respectively. In an embodiment, a GITR agonist comprises V_{H} and V_{L} regions that are each at least 95% identical to the sequences shown in SEQ ID NO:362 and SEQ ID NO:363, respectively.

In an embodiment, a GITR agonist comprises heavy chain CDR1, CDR2 and CDR3 domains having the sequences set forth in SEQ ID NO:364, SEQ ID NO:365, and SEQ ID NO:366, respectively, and conservative amino acid substitutions thereof, and light chain CDR1, CDR2 and CDR3 domains having the sequences set forth in SEQ ID NO:367, SEQ ID NO:368, and SEQ ID NO:369, respectively, and conservative amino acid substitutions thereof.

In an embodiment, the GITR agonist is a GITR agonist biosimilar monoclonal antibody approved by drug regulatory authorities with reference to 48H12. In an embodiment, the biosimilar monoclonal antibody comprises an GITR antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 48H12. In some embodiments, the one or more post-translational modifications are selected from one or more of: glycosylation, oxidation, deamidation, and truncation. In some embodiments, the biosimilar is a GITR agonist antibody authorized or submitted for authorization, wherein the GITR agonist antibody is provided in a formulation which differs from the formulations of a reference medicinal product or reference biological product, wherein the reference medicinal product or reference biological product is 48H12. The GITR agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 48112. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 481112.

TABLE 34
Amino acid sequences for GITR agonist antibodies related to 48H12.

| Identi- | | |
|---|---|---|
| fier | Sequence (One-Letter Amino Acid Symbols) | |
| SEQ ID | QVQLVESGGG VVQPGRSLRL SCAASGFTFS SYGMHWVRQA PGKGLEWVAV IWYAGSNKYY | 60 |
| NO: 360 | ADSVKGRFTI SRDNSKNTVY LQMNSLRAED TAVYYCARGG QLALYYYYGM DVWGQGTTVT | 120 |
| 48H12 | VSSASTKGPS VFPLAPSSKS TSGGTAALGC LVKDYFPEPV TVSWNSGALT SGVHTFPAVL | 180 |
| heavy | QSSGLYSLSS VVTVPSSSLG TQTYICNVNH KPSNTKVDKK VEPKSCDKTH TCPPCPAPEL | 240 |
| chain | LGGPSVFLFP PKPKDTLMIS RTPEVTCVVV DVSHEDPEVK FNWYVDGVEV HNAKTKPREE | 300 |
| | QYNSTYRVVS VLTVLHQDWL NGKEYKCKVS NKALPAPIEK TISKAKGQPR EPQVYTLPPS | 360 |
| | REEMTKNQVS LTCLVKGFYP SDIAVEWESN GQPENNYKTT PPVLDSDGSF FLYSKLTVDK | 420 |
| | SRWQQGNVFS CSVMHEALHN HYTQKSLSLS PGK | 453 |
| SEQ ID | DIQMTQSPSS LSASVGDRVT ITCRASQGIR NDLGWYQQKP GKAPKRLIYA ASSLQSGVPS | 60 |
| NO: 361 | RFSGSGSGTE FTLTISSLQP EDFATYYCLQ HNNYPWTFGQ GTKVEIKRTV AAPSVFIFPP | 120 |
| 48H12 | SDEQLKSGTA SVVCLLNNFY PREAKVQWKV DNALQSGNSQ ESVTEQDSKD STYSLSSTLT | 180 |
| light | LSKADYEKHK VYACEVTHQG LSSPVTKSFN RGEC | 214 |
| chain | | |
| SEQ ID | QVQLVESGGG VVQPGRSLRL SCAASGFTFS SYGMHWVRQA PGKGLEWVAV IWYAGSNKYY | 60 |
| NO: 362 | ADSVKGRFTI SRDNSKNTVY LQMNSLRAED TAVYYCARGG QLALYYYYGM DVWGQGTTVT | 120 |
| 48H12 | VSS | 123 |
| variable | | |
| heavy | | |
| chain | | |
| SEQ ID | DIQMTQSPSS LSASVGDRVT ITCRASQGIR NDLGWYQQKP GKAPKRLIYA ASSLQSGVPS | 60 |
| NO: 363 | RFSGSGSGTE FTLTISSLQP EDFATYYCLQ HNNYPWTFGQ GTKVEIKR | 108 |
| 48H12 | | |
| variable | | |
| light | | |
| chain | | |
| SEQ ID | SYGMH | 5 |
| NO: 364 | | |
| 48H12 | | |
| heavy | | |
| chain | | |
| CDR1 | | |
| SEQ ID | VIWYAGSNKY YADSVKG | 17 |
| NO: 365 | | |
| 48H12 | | |
| heavy | | |
| chain | | |
| CDR2 | | |
| SEQ ID | GGQLALYYYY GMDV | 14 |
| NO: 366 | | |
| 48H12 | | |
| heavy | | |
| chain | | |
| CDR3 | | |
| SEQ ID | RASQGIRNDL G | 11 |
| NO: 367 | | |
| 48H12 | | |
| light | | |
| chain | | |
| CDR1 | | |
| SEQ ID | AASSLQS | 7 |
| NO: 368 | | |
| 48H12 | | |
| light | | |
| chain | | |
| CDR2 | | |
| SEQ ID | LQHNNYPWT | 9 |
| NO: 369 | | |
| 48H12 | | |
| light | | |
| chain | | |
| CDR3 | | |

In a preferred embodiment, the GITR agonist is the monoclonal antibody 48117, or a fragment, derivative, variant, or biosimilar thereof. 48117 is available from Amgen, Inc. The preparation and properties of 48117 are described in U.S. Patent Application Publication No. US 2015/0064204 A1, the disclosures of which are incorporated by reference herein. The amino acid sequences of 48H7 are set forth in Table 35.

In an embodiment, a GITR agonist comprises a heavy chain given by SEQ ID NO:370 and a light chain given by SEQ ID NO:371. In an embodiment, a GITR agonist comprises heavy and light chains having the sequences shown in SEQ ID NO:370 and SEQ ID NO:371, respectively, or antigen binding fragments, Fab fragments, single-chain variable fragments (scFv), variants, or conjugates thereof. In an embodiment, a GITR agonist comprises heavy and light chains that are each at least 99% identical to the sequences shown in SEQ ID NO:370 and SEQ ID NO:371, respectively. In an embodiment, a GITR agonist comprises heavy and light chains that are each at least 98% identical to the sequences shown in SEQ ID NO:370 and SEQ ID NO:371, respectively. In an embodiment, a GITR agonist comprises heavy and light chains that are each at least 97% identical to the sequences shown in SEQ ID NO:370 and SEQ ID NO:371, respectively. In an embodiment, a GITR agonist comprises heavy and light chains that are each at least 96% identical to the sequences shown in SEQ ID NO:370 and SEQ ID NO:371, respectively. In an embodiment, a GITR agonist comprises heavy and light chains that are each at least 95% identical to the sequences shown in SEQ ID NO:370 and SEQ ID NO:371, respectively.

In an embodiment, the GITR agonist comprises the heavy and light chain CDRs or variable regions (VRs) of 48H7. In an embodiment, the GITR agonist heavy chain variable region (V_{H}) comprises the sequence shown in SEQ ID NO:372, and the GITR agonist light chain variable region (V_{L}) comprises the sequence shown in SEQ ID NO:373, and conservative amino acid substitutions thereof. In an embodiment, a GITR agonist comprises V_{H} and V_{L} regions that are each at least 99% identical to the sequences shown in SEQ ID NO:372 and SEQ ID NO:373, respectively. In an embodiment, a GITR agonist comprises V_{H} and V_{L} regions that are each at least 98% identical to the sequences shown in SEQ ID NO:372 and SEQ ID NO:373, respectively. In an embodiment, a GITR agonist comprises V_{H} and V_{L} regions that are each at least 97% identical to the sequences shown in SEQ ID NO:372 and SEQ ID NO:373, respectively. In an embodiment, a GITR agonist comprises V_{H} and V_{L} regions that are each at least 96% identical to the sequences shown in SEQ ID NO:372 and SEQ ID NO:373, respectively. In an embodiment, a GITR agonist comprises V_{H} and V_{L} regions that are each at least 95% identical to the sequences shown in SEQ ID NO:372 and SEQ ID NO:373, respectively.

In an embodiment, a GITR agonist comprises heavy chain CDR1, CDR2 and CDR3 domains having the sequences set forth in SEQ ID NO:374, SEQ ID NO:375, and SEQ ID NO:376, respectively, and conservative amino acid substitutions thereof, and light chain CDR1, CDR2 and CDR3 domains having the sequences set forth in SEQ ID NO:377, SEQ ID NO:378, and SEQ ID NO:379, respectively, and conservative amino acid substitutions thereof.

In an embodiment, the GITR agonist is a GITR agonist biosimilar monoclonal antibody approved by drug regulatory authorities with reference to 48H7. In an embodiment, the biosimilar monoclonal antibody comprises an GITR antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 48H7. In some embodiments, the one or more post-translational modifications are selected from one or more of: glycosylation, oxidation, deamidation, and truncation. In some embodiments, the biosimilar is a GITR agonist antibody authorized or submitted for authorization, wherein the GITR agonist antibody is provided in a formulation which differs from the formulations of a reference medicinal product or reference biological product, wherein the reference medicinal product or reference biological product is 48H7. The GITR agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 48H7. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 48H7.

TABLE 35
Amino acid sequences for GITR agonist antibodies related to 48H7.

| Identi- | | |
|---|---|---|
| fier | Sequence (One-Letter Amino Acid Symbols) | |
| SEQ ID | QVQLVESGGG VVQPGRSLRL SCAASGFTFS SYGMYWVRQA PGKGLEWVAV IWYEGSNKYY | 60 |
| NO: 370 | ADSVKGRFTI SRDNSKNTLY LQMNSLRAED TAVYFCARGG ELGRDYYSGM DVWGQGTTVT | 120 |
| 48H7 | VSSASTKGPS VFPLAPSSKS TSGGTAALGC LVKDYFPEPV TVSWNSGALT SGVHTFPAVL | 180 |
| heavy | QSSGLYSLSS VVTVPSSSLG TQTYICNVNH KPSNTKVDKK VEPKSCDKTH TCPPCPAPEL | 240 |
| chain | LGGPSVFLFP PKPKDTLMIS RTPEVTCVVV DVSHEDPEVK FNWYVDGVEV HNAKTKPREE | 300 |
| | QYNSTYRVVS VLTVLHQDWL NGKEYKCKVS NKALPAPIEK TISKAKGQPR EPQVYTLPPS | 360 |
| | REEMTKNQVS LTCLVKGFYP SDIAVEWESN GQPENNYKTT PPVLDSDGSF FLYSKLTVDK | 420 |
| | SRWQQGNVFS CSVMHEALHN HYTQKSLSLS PGK | 453 |
| SEQ ID | DIQMTQSPSS LSASVGDRVT ITCRASQVIR NDLGWYQQKP GKAPKRLIYA ASSLQSGVPS | 60 |
| NO: 371 | RFSGSGSGTE FTLTISSLQP EDFATYYCLQ HNSYPITFGG GTKVEIKRTV AAPSVFIFPP | 120 |
| 48H7 | SDEQLKSGTA SVVCLLNNFY PREAKVQWKV DNALQSGNSQ ESVTEQDSKD STYSLSSTLT | 180 |
| light | LSKADYEKHK VYACEVTHQG LSSPVTKSFN RGEC | 214 |
| chain | | |
| SEQ ID | QVQLVESGGG VVQPGRSLRL SCAASGFTFS SYGMYWVRQA PGKGLEWVAV IWYEGSNKYY | 60 |
| NO: 372 | ADSVKGRFTI SRDNSKNTLY LQMNSLRAED TAVYFCARGG ELGRDYYSGM DVWGQGTTVT | 120 |
| 48H7 | VSS | 123 |
| variable | | |
| heavy | | |
| chain | | |
| SEQ ID | DIQMTQSPSS LSASVGDRVT ITCRASQVIR NDLGWYQQKP GKAPKRLIYA ASSLQSGVPS | 60 |
| NO: 373 | RFSGSGSGTE FTLTISSLQP EDFATYYCLQ HNSYPITFGG GTKVEIKR | 108 |
| 48H7 | | |
| variable | | |
| light | | |
| chain | | |
| SEQ ID | SYGMY | 5 |
| NO: 374 | | |
| 48H7 | | |
| heavy | | |
| chain | | |
| CDR1 | | |
| SEQ ID | VIWYEGSNKY YADSVKG | 17 |
| NO: 375 | | |
| 48H7 | | |
| heavy | | |
| chain | | |
| CDR2 | | |
| SEQ ID | GGELGRDYYS GMDV | 14 |
| NO: 376 | | |
| 48H7 | | |
| heavy | | |
| chain | | |
| CDR3 | | |
| SEQ ID | RASQVIRNDL G | 11 |
| NO: 377 | | |
| 48H7 | | |
| light | | |
| chain | | |
| CDR1 | | |
| SEQ ID | AASSLQS | 7 |
| NO: 378 | | |
| 48H7 | | |
| light | | |
| chain | | |
| CDR2 | | |
| SEQ ID | LQHNSYPIT | 9 |
| NO: 379 | | |
| 48H7 | | |
| light | | |
| chain | | |
| CDR3 | | |

In a preferred embodiment, the GITR agonist is the monoclonal antibody 49D9, or a fragment, derivative, variant, or biosimilar thereof. 49D9 is available from Amgen, Inc. The preparation and properties of 49D9 are described in U.S. Patent Application Publication No. US 2015/0064204 A1, the disclosures of which are incorporated by reference herein. The amino acid sequences of 49D9 are set forth in Table 36.

In an embodiment, a GITR agonist comprises a heavy chain given by SEQ ID NO:380 and a light chain given by SEQ ID NO:381. In an embodiment, a GITR agonist comprises heavy and light chains having the sequences shown in SEQ ID NO:380 and SEQ ID NO:381, respectively, or antigen binding fragments, Fab fragments, single-chain variable fragments (scFv), variants, or conjugates thereof. In an embodiment, a GITR agonist comprises heavy and light chains that are each at least 99% identical to the sequences shown in SEQ ID NO:380 and SEQ ID NO:381, respectively. In an embodiment, a GITR agonist comprises heavy and light chains that are each at least 98% identical to the sequences shown in SEQ ID NO:380 and SEQ ID NO:381, respectively. In an embodiment, a GITR agonist comprises heavy and light chains that are each at least 97% identical to the sequences shown in SEQ ID NO:380 and SEQ ID NO:381, respectively. In an embodiment, a GITR agonist comprises heavy and light chains that are each at least 96% identical to the sequences shown in SEQ ID NO:380 and SEQ ID NO:381, respectively. In an embodiment, a GITR agonist comprises heavy and light chains that are each at least 95% identical to the sequences shown in SEQ ID NO:380 and SEQ ID NO:381, respectively.

In an embodiment, the GITR agonist comprises the heavy and light chain CDRs or variable regions (VRs) of 49D9. In an embodiment, the GITR agonist heavy chain variable region (V_{H}) comprises the sequence shown in SEQ ID NO:382, and the GITR agonist light chain variable region (V_{L}) comprises the sequence shown in SEQ ID NO:383, and conservative amino acid substitutions thereof. In an embodiment, a GITR agonist comprises V_{H} and V_{L} regions that are each at least 99% identical to the sequences shown in SEQ ID NO:382 and SEQ ID NO:383, respectively. In an embodiment, a GITR agonist comprises V_{H} and V_{L} regions that are each at least 98% identical to the sequences shown in SEQ ID NO:382 and SEQ ID NO:383, respectively. In an embodiment, a GITR agonist comprises V_{H} and V_{L} regions that are each at least 97% identical to the sequences shown in SEQ ID NO:382 and SEQ ID NO:383, respectively. In an embodiment, a GITR agonist comprises V_{H} and V_{L} regions that are each at least 96% identical to the sequences shown in SEQ ID NO:382 and SEQ ID NO:383, respectively. In an embodiment, a GITR agonist comprises V_{H} and V_{L} regions that are each at least 95% identical to the sequences shown in SEQ ID NO:382 and SEQ ID NO:383, respectively.

In an embodiment, a GITR agonist comprises heavy chain CDR1, CDR2 and CDR3 domains having the sequences set forth in SEQ ID NO:384, SEQ ID NO:385, and SEQ ID NO:386, respectively, and conservative amino acid substitutions thereof, and light chain CDR1, CDR2 and CDR3 domains having the sequences set forth in SEQ ID NO:387, SEQ ID NO:388, and SEQ ID NO:389, respectively, and conservative amino acid substitutions thereof.

In an embodiment, the GITR agonist is a GITR agonist biosimilar monoclonal antibody approved by drug regulatory authorities with reference to 49D9. In an embodiment, the biosimilar monoclonal antibody comprises an GITR antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 49D9. In some embodiments, the one or more post-translational modifications are selected from one or more of: glycosylation, oxidation, deamidation, and truncation. In some embodiments, the biosimilar is a GITR agonist antibody authorized or submitted for authorization, wherein the GITR agonist antibody is provided in a formulation which differs from the formulations of a reference medicinal product or reference biological product, wherein the reference medicinal product or reference biological product is 49D9. The GITR agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 49D9. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 49D9.

TABLE 36
Amino acid sequences for GITR agonist antibodies related to 49D9.

| Identi- | | |
|---|---|---|
| fier | Sequence (One-Letter Amino Acid Symbols) | |
| SEQ ID | QMQLVESGGG VVQPGRSLRL SCAASGFTFS SYGMHWVRQA PGKGLEWVAV IWYAGSNKYY | 60 |
| NO: 380 | ADSVKGRFTI SRDNSKNTLY LQMNSLRAED TAVYYCARGG RLGFYYDYGM DVWGQGTTVT | 120 |
| 49D9 | VSSASTKGPS VFPLAPSSKS TSGGTAALGC LVKDYFPEPV TVSWNSGALT SGVHTFPAVL | 180 |
| heavy | QSSGLYSLSS VVTVPSSSLG TQTYICNVNH KPSNTKVDKK VEPKSCDKTH TCPPCPAPEL | 240 |
| chain | LGGPSVFLFP PKPKDTLMIS RTPEVTCVVV DVSHEDPEVK FNWYVDGVEV HNAKTKPREE | 300 |
| | QYNSTYRVVS VLTVLHQDWL NGKEYKCKVS NKALPAPIEK TISKAKGQPR EPQVYTLPPS | 360 |
| | REEMTKNQVS LTCLVKGFYP SDIAVEWESN GQPENNYKTT PPVLDSDGSF FLYSKLTVDK | 420 |
| | SRWQQGNVFS CSVMHEALHN HYTQKSLSLS PGK | 453 |
| SEQ ID | DIQMTQSPSS LSASVGDRVT ITCRASQGIR NDLGWYQQKP GKAPKRLIYA ASSLQSGVPS | 60 |
| NO: 381 | RFSGSGSGTE FTLTISSLQP EDFATYYCLQ LNSYPWTFGQ GTKVEIKRTV AAPSVFIFPP | 120 |
| 49D9 | SDEQLKSGTA SVVCLLNNFY PREAKVQWKV DNALQSGNSQ ESVTEQDSKD STYSLSSTLT | 180 |
| light | LSKADYEKHK VYACEVTHQG LSSPVTKSFN RGEC | 214 |
| chain | | |
| SEQ ID | QMQLVESGGG VVQPGRSLRL SCAASGFTFS SYGMHWVRQA PGKGLEWVAV IWYAGSNKYY | 60 |
| NO: 382 | ADSVKGRFTI SRDNSKNTLY LQMNSLRAED TAVYYCARGG RLGFYYDYGM DVWGQGTTVT | 120 |
| 49D9 | VSS | 123 |
| variable | | |
| heavy | | |
| chain | | |
| SEQ ID | DIQMTQSPSS LSASVGDRVT ITCRASQGIR NDLGWYQQKP GKAPKRLIYA ASSLQSGVPS | 60 |
| NO: 383 | RFSGSGSGTE FTLTISSLQP EDFATYYCLQ LNSYPWTFGQ GTKVEIKR | 108 |
| 49D9 | | |
| variable | | |
| light | | |
| chain | | |
| SEQ ID | SYGMH | 5 |
| NO: 384 | | |
| 49D9 | | |
| heavy | | |
| chain | | |
| CDR1 | | |
| SEQ ID | VIWYAGSNKY YADSVKG | 17 |
| NO: 385 | | |
| 49D9 | | |
| heavy | | |
| chain | | |
| CDR2 | | |
| SEQ ID | GGRLGFYYYY GMDV | 14 |
| NO: 386 | | |
| 49D9 | | |
| heavy | | |
| chain | | |
| CDR3 | | |
| SEQ ID | RASQGIRNDL G | 11 |
| NO: 387 | | |
| 49D9 | | |
| light | | |
| chain | | |
| CDR1 | | |
| SEQ ID | AASSLQS | 7 |
| NO: 388 | | |
| 49D9 | | |
| light | | |
| chain | | |
| CDR2 | | |
| SEQ ID | LQLNSYPWT | 9 |
| NO: 389 | | |
| 49D9 | | |
| light | | |
| chain | | |
| CDR3 | | |

In a preferred embodiment, the GITR agonist is the monoclonal antibody 49E2, or a fragment, derivative, variant, or biosimilar thereof. 49E2 is available from Amgen, Inc. The preparation and properties of 49E2 are described in U.S. Patent Application Publication No. US 2015/0064204 A1, the disclosures of which are incorporated by reference herein. The amino acid sequences of 49E2 are set forth in Table 37.

In an embodiment, a GITR agonist comprises a heavy chain given by SEQ ID NO:390 and a light chain given by SEQ ID NO:391. In an embodiment, a GITR agonist comprises heavy and light chains having the sequences shown in SEQ ID NO:390 and SEQ ID NO:391, respectively, or antigen binding fragments, Fab fragments, single-chain variable fragments (scFv), variants, or conjugates thereof. In an embodiment, a GITR agonist comprises heavy and light chains that are each at least 99% identical to the sequences shown in SEQ ID NO:390 and SEQ ID NO:391, respectively. In an embodiment, a GITR agonist comprises heavy and light chains that are each at least 98% identical to the sequences shown in SEQ ID NO:390 and SEQ ID NO:391, respectively. In an embodiment, a GITR agonist comprises heavy and light chains that are each at least 97% identical to the sequences shown in SEQ ID NO:390 and SEQ ID NO:391, respectively. In an embodiment, a GITR agonist comprises heavy and light chains that are each at least 96% identical to the sequences shown in SEQ ID NO:390 and SEQ ID NO:391, respectively. In an embodiment, a GITR agonist comprises heavy and light chains that are each at least 95% identical to the sequences shown in SEQ ID NO:390 and SEQ ID NO:391, respectively.

In an embodiment, the GITR agonist comprises the heavy and light chain CDRs or variable regions (VRs) of 49E2. In an embodiment, the GITR agonist heavy chain variable region (V_{H}) comprises the sequence shown in SEQ ID NO:392, and the GITR agonist light chain variable region (V_{L}) comprises the sequence shown in SEQ ID NO:393, and conservative amino acid substitutions thereof. In an embodiment, a GITR agonist comprises V_{H} and V_{L} regions that are each at least 99% identical to the sequences shown in SEQ ID NO:392 and SEQ ID NO:393, respectively. In an embodiment, a GITR agonist comprises V_{H} and V_{L} regions that are each at least 98% identical to the sequences shown in SEQ ID NO:392 and SEQ ID NO:393, respectively. In an embodiment, a GITR agonist comprises V_{H} and V_{L} regions that are each at least 97% identical to the sequences shown in SEQ ID NO:392 and SEQ ID NO:393, respectively. In an embodiment, a GITR agonist comprises V_{H} and V_{L} regions that are each at least 96% identical to the sequences shown in SEQ ID NO:392 and SEQ ID NO:393, respectively. In an embodiment, a GITR agonist comprises V_{H} and V_{L} regions that are each at least 95% identical to the sequences shown in SEQ ID NO:392 and SEQ ID NO:393, respectively.

In an embodiment, a GITR agonist comprises heavy chain CDR1, CDR2 and CDR3 domains having the sequences set forth in SEQ ID NO:394, SEQ ID NO:395, and SEQ ID NO:396, respectively, and conservative amino acid substitutions thereof, and light chain CDR1, CDR2 and CDR3 domains having the sequences set forth in SEQ ID NO:397, SEQ ID NO:398, and SEQ ID NO:399, respectively, and conservative amino acid substitutions thereof.

In an embodiment, the GITR agonist is a GITR agonist biosimilar monoclonal antibody approved by drug regulatory authorities with reference to 49E2. In an embodiment, the biosimilar monoclonal antibody comprises an GITR antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 49E2. In some embodiments, the one or more post-translational modifications are selected from one or more of: glycosylation, oxidation, deamidation, and truncation. In some embodiments, the biosimilar is a GITR agonist antibody authorized or submitted for authorization, wherein the GITR agonist antibody is provided in a formulation which differs from the formulations of a reference medicinal product or reference biological product, wherein the reference medicinal product or reference biological product is 49E2. The GITR agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 49E2. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 49E2.

TABLE 37
Amino acid sequences for GITR agonist antibodies related to 49E2.

| Identi- | | |
|---|---|---|
| fier | Sequence (One-Letter Amino Acid Symbols) | |
| SEQ ID | QVQLVESGGG VVQPGRSLRL SCAASGFTFS SYGMHWVRQA PGKGLEWVAV IWSDGNNKYY | 60 |
| NO: 390 | EDSVKGRFTI SRDSSKNTLF LQMNSLRAED TAVYYCARDT ATPFDYWGQG TLVTVSSAST | 120 |
| 49E2 | KGPSVFPLAP SSKSTSGGTA ALGCLVKDYF PEPVTVSWNS GALTSGVHTF PAVLQSSGLY | 180 |
| heavy | SLSSVVTVPS SSLGTQTYIC NVNHKPSNTK VDKKVEPKSC DKTHTCPPCP APELLGGPSV | 240 |
| chain | FLFPPKPKDT LMISRTPEVT CVVVDVSHED PEVKFNWYVD GVEVHNAKTK PREEQYNSTY | 300 |
| | RVVSVLTVLH QDWLNGKEYK CKVSNKALPA PIEKTISKAK GQPREPQVYT LPPSREEMTK | 360 |
| | NQVSLTCLVK GFYPSDIAVE WESNGQPENN YKTTPPVLDS DGSFFLYSKL TVDKSRWQQG | 420 |
| | NVFSCSVMHE ALHNHYTQKS LSLSPGK | 447 |
| SEQ ID | QSALTQPASV SGSPGQSITI SCTGTSSDVG IYNLVSWYQQ HPGKAPKLMI HEVSKRPSGV | 60 |
| NO: 391 | SNRFSGSKSG NTASLTISGL QAEDEADYYC CSYAGISTWV FGGGTKLTVL GQPKAAPSVT | 120 |
| 49E2 | LFPPSSEELQ ANKATLVCLI SDFYPGAVTV AWKADSSPVK AGVETTTPSK QSNNKYAASS | 180 |
| light | YLSLTPEQWK SHRSYSCQVT HEGSTVEKTV APTECS | 216 |
| chain | | |
| SEQ ID | QVQLVESGGG VVQPGRSLRL SCAASGFTFS SYGMHWVRQA PGKGLEWVAV IWSDGNNKYY | 60 |
| NO: 392 | EDSVKGRFTI SRDSSKNTLF LQMNSLRAED TAVYYCARDT ATPFDYWGQG TLVTVSS | 117 |
| 49E2 | | |
| variable | | |
| heavy | | |
| chain | | |
| SEQ ID | QSALTQPASV SGSPGQSITI SCTGTSSDVG IYNLVSWYQQ HPGKAPKLMI HEVSKRPSGV | 60 |
| NO: 393 | SNRFSGSKSG NTASLTISGL QAEDEADYYC CSYAGISTWV FGGGTKLTVL G | 111 |
| 49E2 | | |
| variable | | |
| light | | |
| chain | | |
| SEQ ID | SYGMH | 5 |
| NO: 394 | | |
| 49E2 | | |
| heavy | | |
| chain | | |
| CDR1 | | |
| SEQ ID | VIWSDGNNKY YEDSVKG | 17 |
| NO: 395 | | |
| 49E2 | | |
| heavy | | |
| chain | | |
| CDR2 | | |
| SEQ ID | DTATPFDY | 8 |
| NO: 396 | | |
| 49E2 | | |
| heavy | | |
| chain | | |
| CDR3 | | |
| SEQ ID | TGTSSDVGIY NLVS | 14 |
| NO: 397 | | |
| 49E2 | | |
| light | | |
| chain | | |
| CDR1 | | |
| SEQ ID | EVSKRPS | 7 |
| NO: 398 | | |
| 49E2 | | |
| light | | |
| chain | | |
| CDR2 | | |
| SEQ ID | CSYAGISTWV | 10 |
| NO: 399 | | |
| 49E2 | | |
| light | | |
| chain | | |
| CDR3 | | |

In a preferred embodiment, the GITR agonist is the monoclonal antibody 48A9, or a fragment, derivative, variant, or biosimilar thereof. 48A9 is available from Amgen, Inc. The preparation and properties of 48A9 are described in U.S. Patent Application Publication No. US 2015/0064204 A1, the disclosures of which are incorporated by reference herein. The amino acid sequences of 48A9 are set forth in Table 38.

In an embodiment, a GITR agonist comprises a heavy chain given by SEQ ID NO:400 and a light chain given by SEQ ID NO:401. In an embodiment, a GITR agonist comprises heavy and light chains having the sequences shown in SEQ ID NO:400 and SEQ ID NO:401, respectively, or antigen binding fragments, Fab fragments, single-chain variable fragments (scFv), variants, or conjugates thereof. In an embodiment, a GITR agonist comprises heavy and light chains that are each at least 99% identical to the sequences shown in SEQ ID NO:400 and SEQ ID NO:401, respectively. In an embodiment, a GITR agonist comprises heavy and light chains that are each at least 98% identical to the sequences shown in SEQ ID NO:400 and SEQ ID NO:401, respectively. In an embodiment, a GITR agonist comprises heavy and light chains that are each at least 97% identical to the sequences shown in SEQ ID NO:400 and SEQ ID NO:401, respectively. In an embodiment, a GITR agonist comprises heavy and light chains that are each at least 96% identical to the sequences shown in SEQ ID NO:400 and SEQ ID NO:401, respectively. In an embodiment, a GITR agonist comprises heavy and light chains that are each at least 95% identical to the sequences shown in SEQ ID NO:400 and SEQ ID NO:401, respectively.

In an embodiment, the GITR agonist comprises the heavy and light chain CDRs or variable regions (VRs) of 48A9. In an embodiment, the GITR agonist heavy chain variable region (V_{H}) comprises the sequence shown in SEQ ID NO:402, and the GITR agonist light chain variable region (V_{L}) comprises the sequence shown in SEQ ID NO:403, and conservative amino acid substitutions thereof. In an embodiment, a GITR agonist comprises V_{H} and V_{L} regions that are each at least 99% identical to the sequences shown in SEQ ID NO:402 and SEQ ID NO:403, respectively. In an embodiment, a GITR agonist comprises V_{H} and V_{L} regions that are each at least 98% identical to the sequences shown in SEQ ID NO:402 and SEQ ID NO:403, respectively. In an embodiment, a GITR agonist comprises V_{H} and V_{L} regions that are each at least 97% identical to the sequences shown in SEQ ID NO:402 and SEQ ID NO:403, respectively. In an embodiment, a GITR agonist comprises V_{H} and V_{L} regions that are each at least 96% identical to the sequences shown in SEQ ID NO:402 and SEQ ID NO:403, respectively. In an embodiment, a GITR agonist comprises V_{H} and V_{L} regions that are each at least 95% identical to the sequences shown in SEQ ID NO:402 and SEQ ID NO:403, respectively.

In an embodiment, a GITR agonist comprises heavy chain CDR1, CDR2 and CDR3 domains having the sequences set forth in SEQ ID NO:404, SEQ ID NO:405, and SEQ ID NO:406, respectively, and conservative amino acid substitutions thereof, and light chain CDR1, CDR2 and CDR3 domains having the sequences set forth in SEQ ID NO:407, SEQ ID NO:408, and SEQ ID NO:409, respectively, and conservative amino acid substitutions thereof.

In an embodiment, the GITR agonist is a GITR agonist biosimilar monoclonal antibody approved by drug regulatory authorities with reference to 48A9. In an embodiment, the biosimilar monoclonal antibody comprises an GITR antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 48A9. In some embodiments, the one or more post-translational modifications are selected from one or more of: glycosylation, oxidation, deamidation, and truncation. In some embodiments, the biosimilar is a GITR agonist antibody authorized or submitted for authorization, wherein the GITR agonist antibody is provided in a formulation which differs from the formulations of a reference medicinal product or reference biological product, wherein the reference medicinal product or reference biological product is 48A9. The GITR agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 48A9. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 48A9.

TABLE 38
Amino acid sequences for GITR agonist antibodies related to 48A9.

| Identifier | Sequence (One-Letter Amino Acid Symbols) | |
|---|---|---|
| SEQ ID NO: 400 | QVQLVESGGG VVQPGRSLRL SCAASGFTFS SCGMHWVRQA PGKGLEWVAV ISYDGSNKYY | 60 |
| 48A9 heavy chain | ADSVKGRFTI SRDNSKNTLY LQMNSLRAED TAVYYCARDL RYNWNDGGVD YWGQGTLVTV | 120 |
| | SSASTKGPSV FPLAPSSKST SGGTAALGCL VKDYFPEPVT VSWNSGALTS GVHTFPAVLQ | 180 |
| | SSGLYSLSSV VTVPSSSLGT QTYICNVNHK PSNTKVDKKV EPKSCDKTHT CPPCPAPELL | 240 |
| | GGPSVFLFPP KPKDTLMISR TPEVTCVVVD VSHEDPEVKF NWYVDGVEVH NAKTKPREEQ | 300 |
| | YNSTYRVVSV LTVLHQDWLN GKEYKCKVSN KALPAPIEKT ISKAKGQPRE PQVYTLPPSR | 360 |
| | EEMTKNQVSL TCLVKGFYPS DIAVEWESNG QPENNYKTTP PVLDSDGSFF LYSKLTVDKS | 420 |
| | RWQQGNVFSC SVMHEALHNH YTQKSLSLSP GK | 452 |
| SEQ ID NO: 401 | DIQMTQSPSS LSASVGDRVI ITCRASQSIS SYLHWYKQKP GKAPKLLIYG ASRLQSGVPS | 60 |
| 48A9 light chain | RFSGSGSGTD FTLTISSLQP EDFATYYCQQ SSSTPLTFGG GTKVEIKRTV AAPSVFIFPP | 120 |
| | SDEQLKSGTA SVVCLLNNFY PREAKVQWKV DNALQSGNSQ ESVTEQDSKD STYSLSSTLT | 180 |
| | LSKADYEKHK VYACEVTHQG LSSPVTKSFN RGEC | 214 |
| SEQ ID NO: 402 | QVQLVESGGG VVQPGRSLRL SCAASGFTFS SCGMHWVRQA PGKGLEWVAV ISYDGSNKYY | 60 |
| 48A9 variable | ADSVKGRFTI SRDNSKNTLY LQMNSLRAED TAVYYCARDL RYNWNDGGVD YWGQGTLVTV | 120 |
| heavy chain | SS | 122 |
| SEQ ID NO: 403 | DIQMTQSPSS LSASVGDRVI ITCRASQSIS SYLHWYKQKP GKAPKLLIYG ASRLQSGVPS | 60 |
| 48A9 variable | RFSGSGSGTD FTLTISSLQP EDFATYYCQQ SSSTPLTFGG GTKVEIKR | 108 |
| light chain | | |
| SEQ ID NO: 404 | SCGMH | 5 |
| 48A9 heavy chain | | |
| CDR1 | | |
| SEQ ID NO: 405 | VISYDGSNKY YADSVKG | 17 |
| 48A9 heavy chain | | |
| CDR2 | | |
| SEQ ID NO: 406 | DLRYNWNDGG VDY | 13 |
| 48A9 heavy chain | | |
| CDR3 | | |
| SEQ ID NO: 407 | RASQSISSYL H | 11 |
| 48A9 light chain | | |
| CDR1 | | |
| SEQ ID NO: 408 | GASRLQS | 7 |
| 48A9 light chain | | |
| CDR2 | | |
| SEQ ID NO: 409 | QQSSSTPLT | 9 |
| 48A9 light chain | | |
| CDR3 | | |

In a preferred embodiment, the GITR agonist is the monoclonal antibody 51-17, or a fragment, derivative, variant, or biosimilar thereof. 5117 is available from Amgen, Inc. The preparation and properties of 5117 are described in U.S. Patent Application Publication No. US 2015/0064204 A1, the disclosures of which are incorporated by reference herein. The amino acid sequences of 5117 are set forth in Table 39.

In an embodiment, a GITR agonist comprises a heavy chain given by SEQ ID NO:410 and a light chain given by SEQ TD NO:411. In an embodiment, a GITR agonist comprises heavy and light chains having the sequences shown in SEQ ID NO:410 and SEQ ID NO:411, respectively, or antigen binding fragments, Fab fragments, single-chain variable fragments (scFv), variants, or conjugates thereof. In an embodiment, a GITR agonist comprises heavy and light chains that are each at least 99% identical to the sequences shown in SEQ ID NO:410 and SEQ ID NO:411, respectively. In an embodiment, a GITR agonist comprises heavy and light chains that are each at least 98% identical to the sequences shown in SEQ ID NO:410 and SEQ ID NO:411, respectively. In an embodiment, a GITR agonist comprises heavy and light chains that are each at least 97% identical to the sequences shown in SEQ ID NO:410 and SEQ ID NO:411, respectively. In an embodiment, a GITR agonist comprises heavy and light chains that are each at least 96% identical to the sequences shown in SEQ ID NO:410 and SEQ ID NO:411, respectively. In an embodiment, a GITR agonist comprises heavy and light chains that are each at least 95% identical to the sequences shown in SEQ ID NO:410 and SEQ ID NO:411, respectively.

In an embodiment, the GITR agonist comprises the heavy and light chain CDRs or variable regions (VRs) of 5H7. In an embodiment, the GITR agonist heavy chain variable region (V_{H}) comprises the sequence shown in SEQ ID NO:412, and the GITR agonist light chain variable region (V_{L}) comprises the sequence shown in SEQ ID NO:413, and conservative amino acid substitutions thereof. In an embodiment, a GITR agonist comprises V_{H} and V_{L} regions that are each at least 99% identical to the sequences shown in SEQ ID NO:412 and SEQ ID NO:413, respectively. In an embodiment, a GITR agonist comprises V_{H} and V_{L} regions that are each at least 98% identical to the sequences shown in SEQ ID NO:412 and SEQ ID NO:413, respectively. In an embodiment, a GITR agonist comprises V_{H} and V_{L} regions that are each at least 97% identical to the sequences shown in SEQ ID NO:412 and SEQ ID NO:413, respectively. In an embodiment, a GITR agonist comprises V_{H} and V_{L} regions that are each at least 96% identical to the sequences shown in SEQ ID NO:412 and SEQ ID NO:413, respectively. In an embodiment, a GITR agonist comprises V_{H} and V_{L} regions that are each at least 95% identical to the sequences shown in SEQ ID NO:412 and SEQ ID NO:413, respectively.

In an embodiment, a GITR agonist comprises heavy chain CDR1, CDR2 and CDR3 domains having the sequences set forth in SEQ ID NO:414, SEQ ID NO:415, and SEQ ID NO:416, respectively, and conservative amino acid substitutions thereof, and light chain CDR1, CDR2 and CDR3 domains having the sequences set forth in SEQ ID NO:417, SEQ ID NO:418, and SEQ ID NO:419, respectively, and conservative amino acid substitutions thereof.

In an embodiment, the GITR agonist is a GITR agonist biosimilar monoclonal antibody approved by drug regulatory authorities with reference to 5H7. In an embodiment, the biosimilar monoclonal antibody comprises an GITR antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 5H7. In some embodiments, the one or more post-translational modifications are selected from one or more of: glycosylation, oxidation, deamidation, and truncation. In some embodiments, the biosimilar is a GITR agonist antibody authorized or submitted for authorization, wherein the GITR agonist antibody is provided in a formulation which differs from the formulations of a reference medicinal product or reference biological product, wherein the reference medicinal product or reference biological product is 5H7. The GITR agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 5H7. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 5H7.

TABLE 39
Amino acid sequences for GITR agonist antibodies related to 5H7.

| Identifier | Sequence (One-Letter Amino Acid Symbols) | |
|---|---|---|
| SEQ ID NO: 410 | QVQLQESGPG LVKPSQTLSL TCTVSGGSIS SGGYFWSWIR QHPGKGLEWI GYIYYSGTTY | 60 |
| 5H7 heavy chain | YNPSLKSRVT ISIDTSKNHF SLKLSSVTAA DTAVYYCARD LFYYDSSGPR GFDPWGQGTL | 120 |
| | VTVSSASTKG PSVFPLAPSS KSTSGGTAAL GCLVKDYFPE PVTVSWNSGA LTSGVHTFPA | 180 |
| | VLQSSGLYSL SSVVTVPSSS LGTQTYICNV NHKPSNTKVD KRVEPKSCDK THTCPPCPAP | 240 |
| | ELLGGPSVFL FPPKPKDTLM ISRTPEVTCV VVDVSHEDPE VKFNWYVDGV EVHNAKTKPR | 300 |
| | EEQYNSTYRV VSVLTVLHQD WLNGKEYKCK VSNKALPAPI EKTISKAKGQ PREPQVYTLP | 360 |
| | PSREEMTKNQ VSLTCLVKGF YPSDIAVEWE SNGQPENNYK TTPPVLDSDG SFFLYSKLTV | 420 |
| | DKSRWQQGNV FSCSVMHEAL HNHYTQKSLS LSPGK | 455 |
| SEQ ID NO: 411 | EIVLTQSPGT LSLSPGERAT LSCRASQTVS SNYLAWYQQK PGQAPRLLIY GSSTRATGIP | 60 |
| 5H7 light chain | DRFSGSGSGT DFTLTISRLE PEDFAVYYCQ QYDSSPWTFG QGTKVEIKRT VAAPSVFIFP | 120 |
| | PSDEQLKSGT ASVVCLLNNF YPREAKVQWK VDNALQSGNS QESVTEQDSK DSTYSLSSTL | 180 |
| | TLSKADYEKH KVYACEVTHQ GLSSPVTKSF NRGEC | 215 |
| SEQ ID NO: 412 | QVQLQESGPG LVKPSQTLSL TCTVSGGSIS SGGYFWSWIR QHPGKGLEWI GYIYYSGTTY | 60 |
| 5H7 variable | YNPSLKSRVT ISIDTSKNHF SLKLSSVTAA DTAVYYCARD LFYYDSSGPR GFDPWGQGTL | 120 |
| heavy chain | VTVSS | 125 |
| SEQ ID NO: 413 | EIVLTQSPGT LSLSPGERAT LSCRASQTVS SNYLAWYQQK PGQAPRLLIY GSSTRATGIP | 60 |
| 5H7 variable | DRFSGSGSGT DFTLTISRLE PEDFAVYYCQ QYDSSPWTFG QGTKVEIKR | 109 |
| light chain | | |
| SEQ ID NO: 414 | SGGYFWS | 7 |
| 5H7 heavy chain | | |
| CDR1 | | |
| SEQ ID NO: 415 | YIYYSGTTYY NPSLKS | 16 |
| 5H7 heavy chain | | |
| CDR2 | | |
| SEQ ID NO: 416 | DLFYYDSSGP RGFDP | 15 |
| 5H7 heavy chain | | |
| CDR3 | | |
| SEQ ID NO: 417 | RASQTVSSNYLA | 12 |
| 5H7 light chain | | |
| CDR1 | | |
| SEQ ID NO: 418 | GSSTRAT | 7 |
| 5H7 light chain | | |
| CDR2 | | |
| SEQ ID NO: 419 | QQYDSSPWT | 9 |
| 5H7 light chain | | |
| CDR3 | | |

In a preferred embodiment, the GITR agonist is the monoclonal antibody 7A10, or a fragment, derivative, variant, or biosimilar thereof. 7A10 is available from Amgen, Inc. The preparation and properties of 7A10 are described in U.S. Patent Application Publication No. US 2015/0064204 A1, the disclosures of which are incorporated by reference herein. The amino acid sequences of 7A10 are set forth in Table 40.

In an embodiment, a GITR agonist comprises a heavy chain given by SEQ ID NO:420 and a light chain given by SEQ TD NO:421. In an embodiment, a GITR agonist comprises heavy and light chains having the sequences shown in SEQ ID NO:420 and SEQ TD NO:421, respectively, or antigen binding fragments, Fab fragments, single-chain variable fragments (scFv), variants, or conjugates thereof. In an embodiment, a GITR agonist comprises heavy and light chains that are each at least 9900 identical to the sequences shown in SEQ ID NO:420 and SEQ ID NO:421, respectively. In an embodiment, a GITR agonist comprises heavy and light chains that are each at least 98% identical to the sequences shown in SEQ TD NO:420 and SEQ TD NO:421, respectively. In an embodiment, a GITR agonist comprises heavy and light chains that are each at least 97% identical to the sequences shown in SEQ TD NO:420 and SEQ ID NO:421, respectively. In an embodiment, a GITR agonist comprises heavy and light chains that are each at least 96% identical to the sequences shown in SEQ ID NO:420 and SEQ ID NO:421, respectively. In an embodiment, a GITR agonist comprises heavy and light chains that are each at least 95% identical to the sequences shown in SEQ ID NO:420 and SEQ ID NO:421, respectively.

In an embodiment, the GITR agonist comprises the heavy and light chain CDRs or variable regions (VRs) of 7A10. In an embodiment, the GITR agonist heavy chain variable region (V_{H}) comprises the sequence shown in SEQ ID NO:422, and the GITR agonist light chain variable region (V_{L}) comprises the sequence shown in SEQ ID NO:423, and conservative amino acid substitutions thereof. In an embodiment, a GITR agonist comprises V_{H} and V_{L} regions that are each at least 99% identical to the sequences shown in SEQ ID NO:422 and SEQ ID NO:423, respectively. In an embodiment, a GITR agonist comprises V_{H} and V_{L} regions that are each at least 98% identical to the sequences shown in SEQ ID NO:422 and SEQ ID NO:423, respectively. In an embodiment, a GITR agonist comprises V_{H} and V_{L} regions that are each at least 97% identical to the sequences shown in SEQ ID NO:422 and SEQ ID NO:423, respectively. In an embodiment, a GITR agonist comprises V_{H} and V_{L} regions that are each at least 96% identical to the sequences shown in SEQ ID NO:422 and SEQ ID NO:423, respectively. In an embodiment, a GITR agonist comprises V_{H} and V_{L} regions that are each at least 95% identical to the sequences shown in SEQ ID NO:422 and SEQ ID NO:423, respectively.

In an embodiment, a GITR agonist comprises heavy chain CDR1, CDR2 and CDR3 domains having the sequences set forth in SEQ ID NO:424, SEQ ID NO:425, and SEQ ID NO:426, respectively, and conservative amino acid substitutions thereof, and light chain CDR1, CDR2 and CDR3 domains having the sequences set forth in SEQ ID NO:427, SEQ ID NO:428, and SEQ ID NO:429, respectively, and conservative amino acid substitutions thereof.

In an embodiment, the GITR agonist is a GITR agonist biosimilar monoclonal antibody approved by drug regulatory authorities with reference to 7A10. In an embodiment, the biosimilar monoclonal antibody comprises an GITR antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 7A10. In some embodiments, the one or more post-translational modifications are selected from one or more of: glycosylation, oxidation, deamidation, and truncation. In some embodiments, the biosimilar is a GITR agonist antibody authorized or submitted for authorization, wherein the GITR agonist antibody is provided in a formulation which differs from the formulations of a reference medicinal product or reference biological product, wherein the reference medicinal product or reference biological product is 7A10. The GITR agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 7A10. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 7A10.

TABLE 40
Amino acid sequences for GITR agonist antibodies related to 7A10.

| Identifier | Sequence (One-Letter Amino Acid Symbols) | |
|---|---|---|
| SEQ ID NO: 420 | QVQLVESGGG VVQPGRSLRL SCAASGFTFS SYGMHWVRQA PGKGLEWMAV IWYVGSNKYY | 60 |
| 7A10 heavy chain | ADSVKGRFTI SRDNSKNTLY LQMNSLSAED TAVYYCARGG ELGRDYYSGM DVWGQGTTVT | 120 |
| | VSSASTKGPS VFPLAPSSKS TSGGTAALGC LVKDYFPEPV TVSWNSGALT SGVHTFPAVL | 180 |
| | QSSGLYSLSS VVTVPSSSLG TQTYICNVNH KPSNTKVDKR VEPKSCDKTH TCPPCPAPEL | 240 |
| | LGGPSVFLFP PKPKDTLMIS RTPEVTCVVV DVSHEDPEVK FNWYVDGVEV HNAKTKPREE | 300 |
| | QYNSTYRVVS VLTVLHQDWL NGKEYKCKVS NKALPAPIEK TISKAKGQPR EPQVYTLPPS | 360 |
| | REEMTKNQVS LTCLVKGFYP SDIAVEWESN GQPENNYKTT PPVLDSDGSF FLYSKLTVDK | 420 |
| | SRWQQGNVFS CSVMHEALHN HYTQKSLSLS PGK | 453 |
| SEQ ID NO: 421 | DIQMTQSPSS LSASVGDRVT ITCRASQGIR NDLGWYQQKP GKAPKRLIYA ASSLQSGVPS | 60 |
| 7A10 light chain | RFSGSGSGTE FTLTISSLQP EDFATYYCQQ HNSYPWTFGQ GTKVEIKRTV AAPSVFIFPP | 120 |
| | SDEQLKSGTA SVVCLLNNFY PREAKVQWKV DNALQSGNSQ ESVTEQDSKD STYSLSSTLT | 180 |
| | LSKADYEKHK VYACEVTHQG LSSPVTKSFN RGEC | 214 |
| SEQ ID NO: 422 | QVQLVESGGG VVQPGRSLRL SCAASGFTFS SYGMHWVRQA PGKGLEWMAV IWYVGSNKYY | 60 |
| 7A10 variable | ADSVKGRFTI SRDNSKNTLY LQMNSLSAED TAVYYCARGG ELGRDYYSGM DVWGQGTTVT | 120 |
| heavy chain | VSS | 123 |
| SEQ ID NO: 423 | DIQMTQSPSS LSASVGDRVT ITCRASQGIR NDLGWYQQKP GKAPKRLIYA ASSLQSGVPS | 60 |
| 7A10 variable | RFSGSGSGTE FTLTISSLQP EDFATYYCQQ HNSYPWTFGQ GTKVEIKR | 108 |
| light chain | | |
| SEQ ID NO: 424 | SYGMH | 5 |
| 7A10 heavy chain | | |
| CDR1 | | |
| SEQ ID NO: 425 | VIWYVGSNKY YADSVKG | 17 |
| 7A10 heavy chain | | |
| CDR2 | | |
| SEQ ID NO: 426 | GGELGRDYYS GMDV | 14 |
| 7A10 heavy chain | | |
| CDR3 | | |
| SEQ ID NO: 427 | RASQGIRNDL G | 11 |
| 7A10 light chain | | |
| CDR1 | | |
| SEQ ID NO: 428 | AASSLQS | 7 |
| 7A10 light chain | | |
| CDR2 | | |
| SEQ ID NO: 429 | QQHNSYPWT | 9 |
| 7A10 light chain | | |
| CDR3 | | |

In a preferred embodiment, the GITR agonist is the monoclonal antibody 9H6, or a fragment, derivative, variant, or biosimilar thereof. 9H6 is available from Amgen, Inc. The preparation and properties of 9H6 are described in U.S. Patent Application Publication No. US 2015/0064204 A1, the disclosures of which are incorporated by reference herein. The amino acid sequences of 9H6 are set forth in Table 41.

In an embodiment, a GITR agonist comprises a heavy chain given by SEQ ID NO:430 and a light chain given by SEQ ID NO:431. In an embodiment, a GITR agonist comprises heavy and light chains having the sequences shown in SEQ ID NO:430 and SEQ ID NO:431, respectively, or antigen binding fragments, Fab fragments, single-chain variable fragments (scFv), variants, or conjugates thereof. In an embodiment, a GITR agonist comprises heavy and light chains that are each at least 99% identical to the sequences shown in SEQ ID NO:430 and SEQ ID NO:431, respectively. In an embodiment, a GITR agonist comprises heavy and light chains that are each at least 98% identical to the sequences shown in SEQ ID NO:430 and SEQ ID NO:431, respectively. In an embodiment, a GITR agonist comprises heavy and light chains that are each at least 97% identical to the sequences shown in SEQ ID NO:430 and SEQ ID NO:431, respectively. In an embodiment, a GITR agonist comprises heavy and light chains that are each at least 96% identical to the sequences shown in SEQ ID NO:430 and SEQ ID NO:431, respectively. In an embodiment, a GITR agonist comprises heavy and light chains that are each at least 95% identical to the sequences shown in SEQ ID NO:430 and SEQ ID NO:431, respectively.

In an embodiment, the GITR agonist comprises the heavy and light chain CDRs or variable regions (VRs) of 9H6. In an embodiment, the GITR agonist heavy chain variable region (V_{H}) comprises the sequence shown in SEQ ID NO:432, and the GITR agonist light chain variable region (V_{L}) comprises the sequence shown in SEQ ID NO:433, and conservative amino acid substitutions thereof. In an embodiment, a GITR agonist comprises V_{H} and V_{L} regions that are each at least 99% identical to the sequences shown in SEQ ID NO:432 and SEQ ID NO:433, respectively. In an embodiment, a GITR agonist comprises V_{H} and V_{L} regions that are each at least 98% identical to the sequences shown in SEQ ID NO:432 and SEQ ID NO:433, respectively. In an embodiment, a GITR agonist comprises V_{H} and V_{L} regions that are each at least 97% identical to the sequences shown in SEQ ID NO:432 and SEQ ID NO:433, respectively. In an embodiment, a GITR agonist comprises V_{H} and V_{L} regions that are each at least 96% identical to the sequences shown in SEQ ID NO:432 and SEQ ID NO:433, respectively. In an embodiment, a GITR agonist comprises V_{H} and V_{L} regions that are each at least 95% identical to the sequences shown in SEQ ID NO:432 and SEQ ID NO:433, respectively.

In an embodiment, a GITR agonist comprises heavy chain CDR1, CDR2 and CDR3 domains having the sequences set forth in SEQ ID NO:434, SEQ ID NO:435, and SEQ ID NO:436, respectively, and conservative amino acid substitutions thereof, and light chain CDR1, CDR2 and CDR3 domains having the sequences set forth in SEQ ID NO:437, SEQ ID NO:438, and SEQ ID NO:439, respectively, and conservative amino acid substitutions thereof.

In an embodiment, the GITR agonist is a GITR agonist biosimilar monoclonal antibody approved by drug regulatory authorities with reference to 9H6. In an embodiment, the biosimilar monoclonal antibody comprises an GITR antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 9H6. In some embodiments, the one or more post-translational modifications are selected from one or more of: glycosylation, oxidation, deamidation, and truncation. In some embodiments, the biosimilar is a GITR agonist antibody authorized or submitted for authorization, wherein the GITR agonist antibody is provided in a formulation which differs from the formulations of a reference medicinal product or reference biological product, wherein the reference medicinal product or reference biological product is 9H6. The GITR agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 9116. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 9116.

TABLE 41
Amino acid sequences for GITR agonist antibodies related to 9H6.

| Identifier | Sequence (One-Letter Amino Acid Symbols) | |
|---|---|---|
| SEQ ID NO: 430 | QVQLVESGGG VVQPGRSLRL SCVASGFTFS SYGMHWIRQA PGKGLEWVAV IWYEGSNKYY | 60 |
| 9H6 heavy chain | ADSVKGRFTI SRDNSKNTLY LQMNSLRAED TAVYYCARGG RLGKDYYSGM DVWGQGTTVT | 120 |
| | VSSASTKGPS VFPLAPSSKS TSGGTAALGC LVKDYFPEPV TVSWNSGALT SGVHTFPAVL | 180 |
| | QSSGLYSLSS VVTVPSSSLG TQTYICNVNH KPSNTKVDKR VEPKSCDKTH TCPPCPAPEL | 240 |
| | LGGPSVFLFP PKPKDTLMIS RTPEVTCVVV DVSHEDPEVK FNWYVDGVEV HNAKTKPREE | 300 |
| | QYNSTYRVVS VLTVLHQDWL NGKEYKCKVS NKALPAPIEK TISKAKGQPR EPQVYTLPPS | 360 |
| | REEMTKNQVS LTCLVKGFYP SDIAVEWESN GQPENNYKTT PPVLDSDGSF FLYSKLTVDK | 420 |
| | SRWQQGNVFS CSVMHEALHN HYTQKSLSLS PGK | 453 |
| SEQ ID NO: 431 | DIQMTQSPSS LSASVGDRVT ITCRASQGIR NDLGWYQQKP GKAPNRLIYA TSSLQSGVPS | 60 |
| 9H6 light chain | RFSGSGSGTE FTLTISSLQP EDFATYYCLQ HNTYPWTFGQ GTKVEIKRTV AAPSVFIFPP | 120 |
| | SDEQLKSGTA SVVCLLNNFY PREAKVQWKV DNALQSGNSQ ESVTEQDSKD STYSLSSTLT | 180 |
| | LSKADYEKHK VYACEVTHQG LSSPVTKSFN RGEC | 214 |
| SEQ ID NO: 432 | QVQLVESGGG VVQPGRSLRL SCVASGFTFS SYGMHWIRQA PGKGLEWVAV IWYEGSNKYY | 60 |
| 9H6 variable | ADSVKGRFTI SRDNSKNTLY LQMNSLRAED TAVYYCARGG RLGKDYYSGM DVWGQGTTVT | 120 |
| heavy chain | VSS | 123 |
| SEQ ID NO: 433 | DIQMTQSPSS LSASVGDRVT ITCRASQGIR NDLGWYQQKP GKAPNRLIYA TSSLQSGVPS | 60 |
| 9H6 variable | RFSGSGSGTE FTLTISSLQP EDFATYYCLQ HNTYPWTFGQ GTKVEIKR | 108 |
| light chain | | |
| SEQ ID NO: 434 | SYGMH | 5 |
| 9H6 heavy chain | | |
| CDR1 | | |
| SEQ ID NO: 435 | VIWYEGSNKY YADSVKG | 17 |
| 9H6 heavy chain | | |
| CDR2 | | |
| SEQ ID NO: 436 | GGRLGKDYYS GMDV | 14 |
| 9H6 heavy chain | | |
| CDR3 | | |
| SEQ ID NO: 437 | RASQGIRNDL G | 11 |
| 9H6 light chain | | |
| CDR1 | | |
| SEQ ID NO: 438 | ATSSLQS | 7 |
| 9H6 light chain | | |
| CDR2 | | |
| SEQ ID NO: 439 | LQHNTYPWT | 9 |
| 9H6 light chain | | |
| CDR3 | | |

In an embodiment, the GITR agonist is a GITR agonist described in International Patent Application Publication Nos. WO 2013/039954 A1 and WO 2011/028683 A1; U.S. Patent Application Publication Nos. US 2013/0108641 A1, US 2012/0189639 A1, and US 2014/0348841 A1; and U.S. Pat. Nos. 7,812,135; 8,388,967; and 9,028,823, the disclosures of which are incorporated by reference herein. In an embodiment, the GITR agonist is an agonistic, anti-GITR monoclonal antibody with a structure and preparation described in US Patent Application Publication No. US 2015/0064204 and International Patent Application Publication No. WO 2015/031667 A1 (Amgen, Inc.), the disclosures of which are incorporated by reference herein. In an embodiment, the GITR agonist is a fully-human, agonistic, anti-GITR monoclonal antibody selected from the group consisting of 1D7, 33C9, 33F6, 34G4, 35B10, 41E11, 41G5, 42A11, 44C1, 45A8, 46E11, 48H12, 48H7, 49D9, 49E2, 48A9, 5H7, 7A10, and 9H6. In an embodiment, the GITR agonist is a fully-human, agonistic, anti-GITR monoclonal antibody with an amino acid sequence identity of greater than 99% to the sequence of an antibody selected from the group consisting of 1D7, 33C9, 33F6, 34G4, 35B10, 41E11, 41G5, 42A11, 44C1, 45A8, 46E11, 48H12, 48H7, 49D9, 49E2, 48A9, 5H7, 7A10, and 9H6. In an embodiment, the GITR agonist is a fully-human, agonistic, anti-GITR monoclonal antibody with an amino acid sequence identity of greater than 98% to the sequence of an antibody selected from the group consisting of 1D7, 33C9, 33F6, 34G4, 35B10, 41E11, 41G5, 42A11, 44C1, 45A8, 46E11, 48H12, 48H7, 49D9, 49E2, 48A9, 5H7, 7A10, and 9H6. In an embodiment, the GITR agonist is a fully-human, agonistic, anti-GITR monoclonal antibody selected from the group consisting of 9H6v3, 5H7v2, 33C9v2, 41G5v2, and 7A10v1, as described in US Patent Application Publication No. US 2015/0064204 A1, the disclosure of which is incorporated by reference herein. In an embodiment, the GITR agonist is a fully-human, agonistic, anti-GITR monoclonal antibody selected from the group consisting of 44C1v1, 45A8v1, 49D9v1, 49E2v1, 48A9v1, 5H7v1, 5H7v2, 5H7v3, 5H7v5, 5H7v7, 5H7v9, 5H7v10, 5H7v11, 5H7v13, 5H7v14, 5H7v17, 5H7v18, 5H7v19, 5H7v22, 7A10v1, 7A10v2, 7A10v3, 7A10v4, 7A10v5, 9H6v1, 9H6v2, 9H6v3, 9H6v4, 9H6v5, 9H6v6, 33C9v1, 33C9v2, 33C9v3, 33C9v4, 33C9v5, 41G5v1, 41G5v2, 41G5v3, 41G5v4, and 41G5v5, as described in US Patent Application Publication No. US 2015/0064204 A1, the disclosure of which is incorporated by reference herein.

In an embodiment, the GITR agonist is an GITR agonistic fusion protein as depicted in Structure I-A (C-terminal Fc-antibody fragment fusion protein) or Structure I-B (N-terminal Fc-antibody fragment fusion protein), or a fragment, derivative, conjugate, variant, or biosimilar thereof. The properties of structures I-A and I-B are described above and in U.S. Pat. Nos. 9,359,420, 9,340,599, 8,921,519, and 8,450,460, the disclosures of which are incorporated by reference herein. Amino acid sequences for the polypeptide domains of structure I-A are given in Table 6. The Fc domain preferably comprises a complete constant domain (amino acids 17-230 of SEQ ID NO:31) the complete hinge domain (amino acids 1-16 of SEQ ID NO:31) or a portion of the hinge domain (e.g., amino acids 4-16 of SEQ ID NO:31). Preferred linkers for connecting a C-terminal Fc-antibody may be selected from the embodiments given in SEQ ID NO:32 to SEQ ID NO:41, including linkers suitable for fusion of additional polypeptides. Likewise, amino acid sequences for the polypeptide domains of structure I-B are given in Table 7. If an Fc antibody fragment is fused to the N-terminus of an TNRFSF fusion protein as in structure I-B, the sequence of the Fc module is preferably that shown in SEQ ID NO:42, and the linker sequences are preferably selected from those embodiments set forth in SED ID NO:43 to SEQ ID NO:45.

In an embodiment, an GITR agonist fusion protein according to structures I-A or I-B comprises one or more GITR binding domains selected from the group consisting of a variable heavy chain and variable light chain of TRX518, 6C8, 36E5, 3D6, 61G6, 6H6, 61F6, 1D8, 17F10, 35D8, 49A1, 9E5, 31H6, 2155, 698, 706, 827, 1649, 1718, 1D7, 33C9, 33F6, 34G4, 35B10, 41E11, 41G5, 42A11, 44C1, 45A8, 46E11, 48H12, 48H7, 49D9, 49E2, 48A9, 5H7, 7A10, 9H6, and fragments, derivatives, conjugates, variants, and biosimilars thereof.

In an embodiment, a GITR agonist fusion protein according to structures I-A or I-B comprises one or more GITR binding domains comprising an GITRL sequence (Table 42). In an embodiment, an GITR agonist fusion protein according to structures I-A or I-B comprises one or more GITR binding domains comprising a sequence according to SEQ ID NO:440. In an embodiment, an GITR agonist fusion protein according to structures I-A or I-B comprises one or more GITR binding domains comprising a soluble GITRL sequence. In an embodiment, a GITR agonist fusion protein according to structures I-A or I-B comprises one or more GITR binding domains comprising a sequence according to SEQ ID NO:441.

In an embodiment, an GITR agonist fusion protein according to structures I-A or I-B comprises one or more GITR binding domains that is a scFv domain comprising V_{H} and V_{L} regions that are each at least 95% identical to the V_{H} and V_{L} GITR sequences shown above in Tables 18 to 39, wherein the V_{H} and V_{L} domains are connected by a linker.

TABLE 42
Additional polypeptide domains useful as GITR binding
domains in fusion proteins (e.g., structures I-A and I-B).

| Identifier | Sequence (One-Letter Amino Acid Symbols) | |
|---|---|---|
| SEQ ID NO: 440 | MCLSHLENMP LSHSRTQGAQ RSSWKLWLFC SIVMLLFLCS FSWLIFIFLQ LETAKEPCMA | 60 |
| GITRL | KFGPLPSKWQ MASSEPPCVN KVSDWKLEIL QNGLYLIYGQ VAPNANYNDV APFEVRLYKN | 120 |
| | KDMIQTLTNK SKIQNVGGTY ELHVGDTIDL IFNSEHQVLK NNTYWGIILL ANPQFIS | 177 |
| SEQ ID NO: 441 | TAKEPCMAKF GPLPSKWQMA SSEPPCVNKV SDWKLEILQN GLYLIYGQVA PNANYNDVAP | 60 |
| GITRL soluble | FEVRLYKNKD MIQTLTNKSK IQNVGGTYEL HVGDTIDLIF NSEHQVLKNN TYWGIILLAN | 120 |
| domain | PQFIS | 125 |

In an embodiment, the GITR agonist is a GITR agonistic single-chain fusion polypeptide comprising (i) a first soluble GITR binding domain, (ii) a first peptide linker, (iii) a second soluble GITR binding domain, (iv) a second peptide linker, and (v) a third soluble GITR binding domain, further comprising an additional domain at the N-terminal and/or C-terminal end, and wherein the additional domain is a Fab or Fc fragment domain. In an embodiment, the GITR agonist is a GITR agonistic single-chain fusion polypeptide comprising (i) a first soluble GITR binding domain, (ii) a first peptide linker, (iii) a second soluble GITR binding domain, (iv) a second peptide linker, and (v) a third soluble GITR binding domain, further comprising an additional domain at the N-terminal and/or C-terminal end, wherein the additional domain is a Fab or Fc fragment domain wherein each of the soluble GITR binding domains lacks a stalk region (which contributes to trimerisation and provides a certain distance to the cell membrane, but is not part of the GITR binding domain) and the first and the second peptide linkers independently have a length of 3-8 amino acids.

In an embodiment, the GITR agonist is an GITR agonistic single-chain fusion polypeptide comprising (i) a first soluble tumor necrosis factor (TNF) superfamily cytokine domain, (ii) a first peptide linker, (iii) a second soluble TNF superfamily cytokine domain, (iv) a second peptide linker, and (v) a third soluble TNF superfamily cytokine domain, wherein each of the soluble TNF superfamily cytokine domains lacks a stalk region and the first and the second peptide linkers independently have a length of 3-8 amino acids, and wherein the TNF superfamily cytokine domain is an GITR binding domain.

In an embodiment, the GITR agonist is a GITR agonistic scFv antibody comprising any of the foregoing V_{H} domains linked to any of the foregoing V_{L} domains.

### HVEM (CD270) Agonists

In an embodiment, the TNFRSF agonist is a HVEM agonist. HVEM is also known as CD270 and TNFRSF14. Any HVEM agonist known in the art may be used. The HVEM binding molecule may be a monoclonal antibody or fusion protein capable of binding to human or mammalian HVEM. The HVEM agonists or HVEM binding molecules may comprise an immunoglobulin heavy chain of any isotype (e.g., IgG, IgE, IgM, IgD, IgA, and IgY), class (e.g., IgG1, IgG2, IgG3, IgG4, IgA1 and IgA2) or subclass of immunoglobulin molecule. The HVEM agonist or HVEM binding molecule may have both a heavy and a light chain. As used herein, the term binding molecule also includes antibodies (including full length antibodies), monoclonal antibodies (including full length monoclonal antibodies), polyclonal antibodies, multispecific antibodies (e.g., bispecific antibodies), human, humanized or chimeric antibodies, and antibody fragments, e.g., Fab fragments, F(ab′) fragments, fragments produced by a Fab expression library, epitope-binding fragments of any of the above, and engineered forms of antibodies, e.g., scFv molecules, that bind to HVEM. In an embodiment, the HVEM agonist is an antigen binding protein that is a fully human antibody. In an embodiment, the HVEM agonist is an antigen binding protein that is a humanized antibody. In some embodiments, HVEM agonists for use in the presently disclosed methods and compositions include anti-HVEM antibodies, human anti-HVEM antibodies, mouse anti-HVEM antibodies, mammalian anti-HVEM antibodies, monoclonal anti-HVEM antibodies, polyclonal anti-HVEM antibodies, chimeric anti-HVEM antibodies, anti-HVEM adnectins, anti-HVEM domain antibodies, single chain anti-HVEM fragments, heavy chain anti-HVEM fragments, light chain anti-HVEM fragments, anti-HVEM fusion proteins, and fragments, derivatives, conjugates, variants, or biosimilars thereof. In a preferred embodiment, the HVEM agonist is an agonistic, anti-HVEM humanized or fully human monoclonal antibody (i.e., an antibody derived from a single cell line).

In a preferred embodiment, the HVEM agonist or HVEM binding molecule may also be a fusion protein. In a preferred embodiment, a multimeric HVEM agonist, such as a trimeric or hexameric HVEM agonist (with three or six ligand binding domains), may induce superior receptor (HVEML) clustering and internal cellular signaling complex formation compared to an agonistic monoclonal antibody, which typically possesses two ligand binding domains. Trimeric (trivalent) or hexameric (or hexavalent) or greater fusion proteins comprising three TNFRSF binding domains and IgG1-Fc and optionally further linking two or more of these fusion proteins are described, e.g., in Gieffers, et al., Mol. Cancer Therapeutics 2013, 12, 2735-47.

Agonistic HVEM antibodies and fusion proteins are known to induce strong immune responses. In a preferred embodiment, the HVEM agonist is a monoclonal antibody or fusion protein that binds specifically to HVEM antigen in a manner sufficient to reduce toxicity. In some embodiments, the HVEM agonist is an agonistic HVEM monoclonal antibody or fusion protein that abrogates antibody-dependent cellular toxicity (ADCC), for example NK cell cytotoxicity. In some embodiments, the HVEM agonist is an agonistic HVEM monoclonal antibody or fusion protein that abrogates antibody-dependent cell phagocytosis (ADCP). In some embodiments, the HVEM agonist is an agonistic HVEM monoclonal antibody or fusion protein that abrogates complement-dependent cytotoxicity (CDC). In some embodiments, the HVEM agonist is an agonistic HVEM monoclonal antibody or fusion protein which abrogates Fc region functionality.

In some embodiments, the HVEM agonists are characterized by binding to human HVEM (SEQ ID NO:442) with high affinity and agonistic activity. In an embodiment, the HVEM agonist is a binding molecule that binds to human HVEM (SEQ ID NO:442). The amino acid sequence of HVEM antigen to which a HVEM agonist or binding molecule may bind is summarized in Table 43.

TABLE 43
Amino acid sequence of HVEM (CD270) antigen.

| Identifier | Sequence (One-Letter Amino Acid Symbols) | |
|---|---|---|
| SEQ ID NO: 442 | MEPPGDWGPP PWRSTPRTDV LRLVLYLTFL GAPCYAPALP SCKEDEYPVG SECCPKCSPG | 60 |
| human CD270, | YRVKEACGEL TGTVCEPCPP GTYIAHLNGL SKCLQCQMCD PAMGLRASRN CSRTENAVCG | 120 |
| Tumor necrosis | CSPGHFCIVQ DGDHCAACRA YATSSPGQRV QKGGTESQDT LCQNCPPGTF SPNGTLEECQ | 180 |
| factor receptor | HQTKCSWLVT KAGAGTSSSH WVWWFLSGSL VIVIVCSTVG LIICVKRRKP RGDVVKVIVS | 240 |
| superfamily, | VQRKRQEAEG EATVIEALQA PPDVTTVAVE ETIPSFTGRS PNH | 283 |
| member 14 (Homo | | |
| sapiens) | | |

In some embodiments, the compositions, processes and methods described include a HVEM agonist that binds human or murine HVEM with a K_{D} of about 100 pM or lower, binds human or murine HVEM with a K_{D} of about 90 pM or lower, binds human or murine HVEM with a K_{D} of about 80 pM or lower, binds human or murine HVEM with a K_{D} of about 70 pM or lower, binds human or murine HVEM with a K_{D} of about 60 pM or lower, binds human or murine HVEM with a K_{D} of about 50 pM or lower, binds human or murine HVEM with a K_{D} of about 40 pM or lower, or binds human or murine HVEM with a K_{D} of about 30 pM or lower.

In some embodiments, the compositions, processes and methods described include a HVEM agonist that binds to human or murine HVEM with a k_{assoc} of about 7.5×10⁵ l/M·s or faster, binds to human or murine HVEM with a k_{assoc} of about 7.5×10⁵ l/M·s or faster, binds to human or murine HVEM with a k_{assoc} of about 8×10⁵ l/M·s or faster, binds to human or murine HVEM with a k_{assoc} of about 8.5×10⁵ l/M·s or faster, binds to human or murine HVEM with a k_{assoc} of about 9×10⁵ l/M·s or faster, binds to human or murine HVEM with a k_{assoc} of about 9.5×10⁵ l/M·s or faster, or binds to human or murine HVEM with a k_{assoc} of about 1×10⁶ l/M·s or faster.

In some embodiments, the compositions, processes and methods described include a HVEM agonist that binds to human or murine HVEM with a k_{dissoc} of about 2×10⁻⁵ l/s or slower, binds to human or murine HVEM with a k_{dissoc} of about 2.1×10⁻⁵ l/s or slower, binds to human or murine HVEM with a k_{dissoc} of about 2.2×10⁻⁵ l/s or slower, binds to human or murine HVEM with a k_{dissoc} of about 2.3×10⁻⁵ l/s or slower, binds to human or murine HVEM with a k_{dissoc} of about 2.4×10⁻⁵ l/s or slower, binds to human or murine HVEM with a k_{dissoc} of about 2.5×10⁻⁵ l/s or slower, binds to human or murine HVEM with a k_{dissoc} of about 2.6×10⁻⁵ l/s or slower or binds to human or murine HVEM with a k_{dissoc} of about 2.7×10⁻⁵ l/s or slower, binds to human or murine HVEM with a k_{dissoc} of about 2.8×10⁻⁵ l/s or slower, binds to human or murine HVEM with a k_{dissoc} of about 2.9×10⁻⁵ l/s or slower, or binds to human or murine HVEM with a k_{dissoc} of about 3×10⁻⁵ l/s or slower.

In some embodiments, the compositions, processes and methods described include a HVEM agonist that binds to human or murine HVEM with an IC₅₀ of about 10 nM or lower, binds to human or murine HVEM with an IC₅₀ of about 9 nM or lower, binds to human or murine HVEM with an IC₅₀ of about 8 nM or lower, binds to human or murine HVEM with an IC₅₀ of about 7 nM or lower, binds to human or murine HVEM with an IC₅₀ of about 6 nM or lower, binds to human or murine HVEM with an IC₅₀ of about 5 nM or lower, binds to human or murine HVEM with an IC₅₀ of about 4 nM or lower, binds to human or murine HVEM with an IC₅₀ of about 3 nM or lower, binds to human or murine HVEM with an IC₅₀ of about 2 nM or lower, or binds to human or murine HVEM with an IC₅₀ of about 1 nM or lower.

In an embodiment, the HVEM agonist is an HVEM agonist described in International Patent Application Publication No. WO 2009/007120 A2 and U.S. Patent Application Publication No. US 2016/0176941 A1, the disclosure of each of which is incorporated by reference herein.

In an embodiment, the HVEM agonist is the HVEM agonist clone REA247, which is commercially available from Miltenyi Biotech, Inc. (San Diego, CA 92121).

In an embodiment, the HVEM agonist is an HVEM agonistic fusion protein as depicted in Structure I-A (C-terminal Fc-antibody fragment fusion protein) or Structure I-B (N-terminal Fc-antibody fragment fusion protein), or a fragment, derivative, conjugate, variant, or biosimilar thereof. The properties of structures I-A and I-B are described above and in U.S. Pat. Nos. 9,359,420, 9,340,599, 8,921,519, and 8,450,460, the disclosures of which are incorporated by reference herein. Amino acid sequences for the polypeptide domains of structure I-A are given in Table 6. The Fc domain preferably comprises a complete constant domain (amino acids 17-230 of SEQ ID NO:31) the complete hinge domain (amino acids 1-16 of SEQ ID NO:31) or a portion of the hinge domain (e.g., amino acids 4-16 of SEQ ID NO:31). Preferred linkers for connecting a C-terminal Fc-antibody may be selected from the embodiments given in SEQ ID NO:32 to SEQ ID NO:41, including linkers suitable for fusion of additional polypeptides. Likewise, amino acid sequences for the polypeptide domains of structure I-B are given in Table 7. If an Fc antibody fragment is fused to the N-terminus of an TNRFSF fusion protein as in structure I-B, the sequence of the Fc module is preferably that shown in SEQ ID NO:42, and the linker sequences are preferably selected from those embodiments set forth in SED ID NO:43 to SEQ ID NO:45.

In an embodiment, an HVEM agonist fusion protein according to structures I-A or I-B comprises one or more HVEM binding domains comprising an LIGHT (HVEM ligand) sequence (Table 44). In an embodiment, an HVEM agonist fusion protein according to structures I-A or I-B comprises one or more HVEM binding domains comprising a sequence according to SEQ ID NO:443. In an embodiment, an HVEM agonist fusion protein according to structures I-A or I-B comprises one or more HVEM binding domains comprising a soluble LIGHT sequence. In an embodiment, a HVEM agonist fusion protein according to structures I-A or I-B comprises one or more HVEM binding domains comprising a sequence according to SEQ ID NO:444. In an embodiment, a HVEM agonist fusion protein according to structures I-A or I-B comprises one or more HVEM binding domains comprising a sequence according to SEQ ID NO:445. In an embodiment, a HVEM agonist fusion protein according to structures I-A or I-B comprises one or more HVEM binding domains comprising a sequence according to SEQ ID NO:446.

In an embodiment, an HVEM agonist fusion protein according to structures I-A or I-B comprises one or more HVEM binding domains that is a scFv domain comprising V_{H} and V_{L} regions, wherein the V_{H} and V_{L} domains are connected by a linker.

TABLE 44
Additional polypeptide domains useful as HVEM binding
domains in fusion proteins (e.g., structures I-A and I-B).

| Identifier | Sequence (One-Letter Amino Acid Symbols) | |
|---|---|---|
| SEQ ID NO: 443 | MEESVVRPSV FVVDGQTDIP FTRLGRSHRR QSCSVARVGL GLLLLLMGAG LAVQGWFLLQ | 60 |
| LIGHT (HVEM | LHWRLGEMVT RLPDGPAGSW EQLIQERRSH EVNPAAHLTG ANSSLTGSGG PLLWETQLGL | 120 |
| ligand) | AFLRGLSYHD GALVVTKAGY YYIYSKVQLG GVGCPLGLAS TITHGLYKRT PRYPEELELL | 180 |
| | VSQQSPCGRA TSSSRVWWDS SFLGGVVHLE AGEKVVVRVL DERLVRLRDG TRSYFGAFMV | 240 |
| SEQ ID NO: 444 | PAAHLTGANS SLTGSGGPLL WETQLGLAFL RGLSYHDGAL VVTKAGYYYI YSKVQLGGVG | 60 |
| LIGHT soluble | CPLGLASTIT HGLYKRTPRY PEELELLVSQ QSPCGRATSS SRVWWDSSFL GGVVHLEAGE | 120 |
| domain | KVVVRVLDER LVRLRDGTRS YFGAFMV | 147 |
| SEQ ID NO: 445 | AAHLTGANSS LTGSGGPLLW ETQLGLAFLR GLSYHDGALV VTKAGYYYIY SKVQLGGVGC | 60 |
| LIGHT soluble | PLGLASTITH GLYKRTPRYP EELELLVSQQ SPCGRATSSS RVWWDSSFLG GVVHLEAGEK | 120 |
| domain | VVVRVLDERL VRLRDGTRSY FGAFMV | 146 |
| (alternative) | | |
| SEQ ID NO: 446 | AHLTGANSSL TGSGGPLLWE TQLGLAFLRG LSYHDGALVV TKAGYYYIYS KVQLGGVGCP | 60 |
| LIGHT soluble | LGLASTITHG LYKRTPRYPE ELELLVSQQS PCGRATSSSR VWWDSSFLGG VVHLEAGEKV | 120 |
| domain | VVRVIDERLV RLRDGTRSYF GAFMV | 145 |
| (alternative) | | |

In an embodiment, the HVEM agonist is a HVEM agonistic single-chain fusion polypeptide comprising (i) a first soluble HVEM binding domain, (ii) a first peptide linker, (iii) a second soluble HVEM binding domain, (iv) a second peptide linker, and (v) a third soluble HVEM binding domain, further comprising an additional domain at the N-terminal and/or C-terminal end, and wherein the additional domain is a Fab or Fc fragment domain. In an embodiment, the HVEM agonist is a HVEM agonistic single-chain fusion polypeptide comprising (i) a first soluble HVEM binding domain, (ii) a first peptide linker, (iii) a second soluble HVEM binding domain, (iv) a second peptide linker, and (v) a third soluble HVEM binding domain, further comprising an additional domain at the N-terminal and/or C-terminal end, wherein the additional domain is a Fab or Fc fragment domain wherein each of the soluble HVEM binding domains lacks a stalk region (which contributes to trimerisation and provides a certain distance to the cell membrane, but is not part of the HVEM binding domain) and the first and the second peptide linkers independently have a length of 3-8 amino acids.

In an embodiment, the HVEM agonist is an HVEM agonistic single-chain fusion polypeptide comprising (i) a first soluble tumor necrosis factor (TNF) superfamily cytokine domain, (ii) a first peptide linker, (iii) a second soluble TNF superfamily cytokine domain, (iv) a second peptide linker, and (v) a third soluble TNF superfamily cytokine domain, wherein each of the soluble TNF superfamily cytokine domains lacks a stalk region and the first and the second peptide linkers independently have a length of 3-8 amino acids, and wherein the TNF superfamily cytokine domain is an HVEM binding domain.

In an embodiment, the HVEM agonist is a HVEM agonist described in U.S. Pat. No. 7,118,742, the disclosure of which is incorporated by reference herein.

### CD95 Agonists

In an embodiment, the TNFRSF agonist is a CD95 agonist or CD95 binding molecule. CD95 is also known as TNFRSF6, Fas receptor (FasR), and APO-1. Any CD95 agonist or binding molecule known in the art may be used. The CD95 binding molecule may be a monoclonal antibody or fusion protein capable of binding to human or mammalian CD95, and may be used at a concentration appropriate for T cell agonistic activity rather than T cell apoptotic activity, as described elsewhere herein. The CD95 agonists or CD95 binding molecules may comprise an immunoglobulin heavy chain of any isotype (e.g., IgG, IgE, IgM, IgD, IgA, and IgY), class (e.g., IgG1, IgG2, IgG3, IgG4, IgA1 and IgA2) or subclass of immunoglobulin molecule. The CD95 agonist or CD95 binding molecule may have both a heavy and a light chain. As used herein, the term binding molecule also includes antibodies (including full length antibodies), monoclonal antibodies (including full length monoclonal antibodies), polyclonal antibodies, multispecific antibodies (e.g., bispecific antibodies), human, humanized or chimeric antibodies, and antibody fragments, e.g., Fab fragments, F(ab′) fragments, fragments produced by a Fab expression library, epitope-binding fragments of any of the above, and engineered forms of antibodies, e.g., scFv molecules, that bind to CD95. In an embodiment, the CD95 agonist is an antigen binding protein that is a fully human antibody. In an embodiment, the CD95 agonist is an antigen binding protein that is a humanized antibody. In some embodiments, CD95 agonists for use in the presently disclosed methods and compositions include anti-CD95 antibodies, human anti-CD95 antibodies, mouse anti-CD95 antibodies, mammalian anti-CD95 antibodies, monoclonal anti-CD95 antibodies, polyclonal anti-CD95 antibodies, chimeric anti-CD95 antibodies, anti-CD95 adnectins, anti-CD95 domain antibodies, single chain anti-CD95 fragments, heavy chain anti-CD95 fragments, light chain anti-CD95 fragments, anti-CD95 fusion proteins, and fragments, derivatives, conjugates, variants, or biosimilars thereof. In a preferred embodiment, the CD95 agonist is an agonistic, anti-CD95 humanized or fully human monoclonal antibody (i.e., an antibody derived from a single cell line).

In a preferred embodiment, the CD95 agonist or CD95 binding molecule may also be a fusion protein. In a preferred embodiment, a multimeric CD95 agonist, such as a trimeric or hexameric CD95 agonist (with three or six ligand binding domains), may induce superior receptor (CD95L) clustering and internal cellular signaling complex formation compared to an agonistic monoclonal antibody, which typically possesses two ligand binding domains. Trimeric (trivalent) or hexameric (or hexavalent) or greater fusion proteins comprising three TNFRSF binding domains and IgG1-Fc and optionally further linking two or more of these fusion proteins are described, e.g., in Gieffers, et al., Mol. Cancer Therapeutics 2013, 12, 2735-47.

Agonistic CD95 antibodies and fusion proteins are known to induce strong immune responses. In a preferred embodiment, the CD95 agonist is a monoclonal antibody or fusion protein that binds specifically to CD95 antigen in a manner sufficient to reduce toxicity. In some embodiments, the CD95 agonist is an agonistic CD95 monoclonal antibody or fusion protein that abrogates antibody-dependent cellular toxicity (ADCC), for example NK cell cytotoxicity. In some embodiments, the CD95 agonist is an agonistic CD95 monoclonal antibody or fusion protein that abrogates antibody-dependent cell phagocytosis (ADCP). In some embodiments, the CD95 agonist is an agonistic CD95 monoclonal antibody or fusion protein that abrogates complement-dependent cytotoxicity (CDC). In some embodiments, the CD95 agonist is an agonistic CD95 monoclonal antibody or fusion protein which abrogates Fc region functionality.

In some embodiments, the CD95 agonists are characterized by binding to human CD95 (SEQ ID NO:447) with high affinity and agonistic activity. In an embodiment, the CD95 agonist is a binding molecule that binds to human CD95 (SEQ ID NO:447). In an embodiment, the CD95 agonist is a binding molecule that binds to human CD95 (SEQ ID NO:448). In an embodiment, the CD95 agonist is a binding molecule that binds to human CD95 (SEQ ID NO:449). In an embodiment, the CD95 agonist is a binding molecule that binds to human CD95 (SEQ ID NO:450). The amino acid sequence of CD95 antigens to which a CD95 agonist or binding molecule may bind is summarized in Table 45.

TABLE 45
Amino acid sequence of CD95 antigens.

| Identifier | Sequence (One-Letter Amino Acid Symbols) | |
|---|---|---|
| SEQ ID NO: 447 | MLGIWTLLPL VLTSVARLSS KSVNAQVTDI NSKGLELRKT VTTVETQNLE GLHHDGQFCH | 60 |
| human CD95, | KPCPPGERKA RDCTVNGDEP DCVPCQEGKE YTDKAHFSSK CRRCRLCDEG HGLEVEINCT | 120 |
| Tumor necrosis | RTQNTKCRCK PNFFCNSTVC EHCDPCTKCE HGIIKECTLT SNTKCKEEGS RSNLGWLCLL | 180 |
| factor receptor | LLPIPLIVWV KRKEVQKTCR KHRKENQGSH ESPTLNPETV AINLSDVDLS KYITTIAGVM | 240 |
| superfamily, | TLSQVKGFVR KNGVNEAKID EIKNDNVQDT AEQKVQLLRN WHQLHGKKEA YDTLIKDLKK | 300 |
| member 6 (Homo | ANLCTLAEKI QTIILKDITS DSENSNFRNE IQSLV | 335 |
| sapiens), | | |
| isoform 1 | | |
| SEQ ID NO: 448 | MLGIWTLLPL VLTSVARLSS KSVNAQVTDI NSKGLELRKT VTTVETQNLE GLHHDGQFCH | 60 |
| human CD95, | KPCPPGERKA RDCTVNGDEP DCVPCQEGKE YTDKAHFSSK CRRCRLCDEG HGLEVEINCT | 120 |
| Tumor necrosis | RTQNTKCRCK PNFFCNSTVC EHCDPCTKCE HGIIKECTLT SNTKCKEEVK RKEVQKTCRK | 180 |
| factor receptor | HRKENQGSHE SPTLNPETVA INLSDVDLSK YITTIAGVMT LSQVKGFVRK NGVNEAKIDE | 240 |
| superfamily, | IKNDNVQDTA EQKVQLLRNW HQLHGKKEAY DTLIKDLKKA NLCTLAEKIQ TIILKDITSD | 300 |
| member 6 (Homo | SENSNFRNEI QSLV | 314 |
| sapiens), | | |
| isoform 2 | | |
| SEQ ID NO: 449 | MLGIWTLLPL VLTSVARLSS KSVNAQVTDI NSKGLELRKT VTTVETQNLE GLHHDGQFCH | 60 |
| human CD95, | KPCPPGERKA RDCTVNGDEP DCVPCQEGKE YTDKAHFSSK CRRCRLCDEG HGLEVEINCT | 120 |
| Tumor necrosis | RTQNTKCRCK PNFFCNSTVC EHCDPCTKCE HGIIKECTLT SNTKCKEEGS RSNLGWLCLL | 180 |
| factor receptor | LLPIPLIVWV KRKEVQKTCR KHRKENQGSH ESPTLNPMLT | 220 |
| superfamily, | | |
| member 6 (Homo | | |
| sapiens), | | |
| isoform 3 | | |
| SEQ ID NO: 450 | MLGIWTLLPL VLTSVARLSS KSVNAQVTDI NSKGLELRKT VTTVETQNLE GLHHDGQFCH | 60 |
| human CD95, | KPCPPGERKA RDCTVNGDEP DCVPCQEGKE YTDKAHFSSK CRRCRLCDEG HGLEVEINCT | 120 |
| Tumor necrosis | RTQNTKCRCK PNFFCNSTVC EHCDPCTKCE HGIIKECTLT SNTKCKEEGS RSNLGWLCLL | 180 |
| factor receptor | LLPIPLIVWG NSGNKFI | 197 |
| superfamily, | | |
| member 6 (Homo | | |
| sapiens), | | |
| isoform 4 | | |

In some embodiments, the compositions, processes and methods described include a CD95 agonist that binds human or murine CD95 with a K_{D} of about 100 pM or lower, binds human or murine CD95 with a K_{D} of about 90 pM or lower, binds human or murine CD95 with a K_{D} of about 80 pM or lower, binds human or murine CD95 with a K_{D} of about 70 pM or lower, binds human or murine CD95 with a K_{D} of about 60 pM or lower, binds human or murine CD95 with a K_{D} of about 50 pM or lower, binds human or murine CD95 with a K_{D} of about 40 pM or lower, or binds human or murine CD95 with a K_{D} of about 30 pM or lower.

In some embodiments, the compositions, processes and methods described include a CD95 agonist that binds to human or murine CD95 with a k_{assoc} of about 7.5×10⁵ l/M·s or faster, binds to human or murine CD95 with a k_{assoc} of about 7.5×10⁵ l/M·s or faster, binds to human or murine CD95 with a k_{assoc} of about 8×10⁵ l/M·s or faster, binds to human or murine CD95 with a k_{assoc} of about 8.5×10⁵ l/M·s or faster, binds to human or murine CD95 with a k_{assoc} of about 9×10⁵ l/M·s or faster, binds to human or murine CD95 with a k_{assoc} of about 9.5×10⁵ l/M·s or faster, or binds to human or murine CD95 with a k_{assoc} of about 1×10⁶ l/M·s or faster.

In some embodiments, the compositions, processes and methods described include a CD95 agonist that binds to human or murine CD95 with a k_{dissoc} of about 2×10⁻⁵ l/s or slower, binds to human or murine CD95 with a k_{dissoc} of about 2.1×10⁻⁵ l/s or slower, binds to human or murine CD95 with a k_{dissoc} of about 2.2×10⁻⁵ l/s or slower, binds to human or murine CD95 with a k_{dissoc} of about 2.3×10⁻⁵ l/s or slower, binds to human or murine CD95 with a k_{dissoc} of about 2.4×10⁻⁵ l/s or slower, binds to human or murine CD95 with a k_{dissoc} of about 2.5×10⁻⁵ l/s or slower, binds to human or murine CD95 with a k_{dissoc} of about 2.6×10⁻⁵ l/s or slower or binds to human or murine CD95 with a k_{dissoc} of about 2.7×10⁻⁵ l/s or slower, binds to human or murine CD95 with a k_{dissoc} of about 2.8×10⁻⁵ l/s or slower, binds to human or murine CD95 with a k_{dissoc} of about 2.9×10⁻⁵ l/s or slower, or binds to human or murine CD95 with a k_{dissoc} of about 3×10⁻⁵ l/s or slower.

In some embodiments, the compositions, processes and methods described include a CD95 agonist that binds to human or murine CD95 with an IC₅₀ of about 10 nM or lower, binds to human or murine CD95 with an IC₅₀ of about 9 nM or lower, binds to human or murine CD95 with an IC₅₀ of about 8 nM or lower, binds to human or murine CD95 with an IC₅₀ of about 7 nM or lower, binds to human or murine CD95 with an IC₅₀ of about 6 nM or lower, binds to human or murine CD95 with an IC₅₀ of about 5 nM or lower, binds to human or murine CD95 with an IC₅₀ of about 4 nM or lower, binds to human or murine CD95 with an IC₅₀ of about 3 nM or lower, binds to human or murine CD95 with an IC₅₀ of about 2 nM or lower, or binds to human or murine CD95 with an IC₅₀ of about 1 nM or lower.

In a preferred embodiment, the CD95 agonist is the monoclonal antibody E09, or a fragment, derivative, variant, or biosimilar thereof. The preparation and properties of E09 are described in Chodorge, et al., Cell Death & Differ. 2012, 19, 1187-95. The amino acid sequences of E09 are set forth in Table 46.

In an embodiment, the CD95 agonist comprises the heavy and light chain CDRs or variable regions (VRs) of E09. In an embodiment, the CD95 agonist heavy chain variable region (V_{H}) comprises the sequence shown in SEQ ID NO:451, and the CD95 agonist light chain variable region (V_{L}) comprises the sequence shown in SEQ ID NO:452, and conservative amino acid substitutions thereof. In an embodiment, a CD95 agonist comprises V_{H} and V_{L} regions that are each at least 99% identical to the sequences shown in SEQ ID NO:451 and SEQ ID NO:452, respectively. In an embodiment, a CD95 agonist comprises V_{H} and V_{L} regions that are each at least 98% identical to the sequences shown in SEQ ID NO:451 and SEQ ID NO:452, respectively. In an embodiment, a CD95 agonist comprises V_{H} and V_{L} regions that are each at least 97% identical to the sequences shown in SEQ ID NO:451 and SEQ ID NO:452, respectively. In an embodiment, a CD95 agonist comprises V_{H} and V_{L} regions that are each at least 96% identical to the sequences shown in SEQ ID NO:451 and SEQ ID NO:452, respectively. In an embodiment, a CD95 agonist comprises V_{H} and V_{L} regions that are each at least 95% identical to the sequences shown in SEQ ID NO:451 and SEQ ID NO:452, respectively.

In an embodiment, a CD95 agonist comprises heavy chain CDR1, CDR2 and CDR3 domains having the sequences set forth in SEQ ID NO:453, SEQ ID NO:454, and SEQ ID NO:455, respectively, and conservative amino acid substitutions thereof, and light chain CDR1, CDR2 and CDR3 domains having the sequences set forth in SEQ ID NO:456, SEQ ID NO:457, and SEQ ID NO:458, respectively, and conservative amino acid substitutions thereof.

In an embodiment, the CD95 agonist is a CD95 agonist biosimilar monoclonal antibody approved by drug regulatory authorities with reference to E09. In an embodiment, the biosimilar monoclonal antibody comprises an CD95 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is E09. In some embodiments, the one or more post-translational modifications are selected from one or more of: glycosylation, oxidation, deamidation, and truncation. In some embodiments, the biosimilar is a CD95 agonist antibody authorized or submitted for authorization, wherein the CD95 agonist antibody is provided in a formulation which differs from the formulations of a reference medicinal product or reference biological product, wherein the reference medicinal product or reference biological product is E09. The CD95 agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is E09. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is E09.

TABLE 46
Amino acid sequences for CD95 agonist antibody E09.

| Identifier | Sequence (One-Letter Amino Acid Symbols) | |
|---|---|---|
| SEQ ID NO: 451 | QLQLQESGPG LVKPSETLSL TCTVSGASIS ANSYYGVWVR QSPGKGLEWV GSIAYRGNSN | 60 |
| heavy chain | SGSTYYNPSL KSRATVSVDT SKNQVSLRLT SVTAADTALY YCARRQLLDD GTGYQWAAFD | 120 |
| variable region | VWGQGTMVTV SS | 132 |
| for E09 | | |
| SEQ ID NO: 452 | QSVLTQPPSV SEAPRQTVTI SCSGNSFNIG RYPVNWYQQL PGKAPKLLIY YNNLRFSGVS | 60 |
| light chain | DRFSGSKSGT SASLAIRDLL SEDEADYYCS TWDDTLKGWV FGGGTKVTVL | 110 |
| variable region | | |
| for E09 | | |
| SEQ ID NO: 453 | ANSYYGV | 7 |
| heavy chain | | |
| CDR1 for E09 | | |
| SEQ ID NO: 454 | GSIAYRGNSN SGSTYYNPSL KS | 22 |
| heavy chain | | |
| CDR2 for E09 | | |
| SEQ ID NO: 455 | RQLLDDGTGY QWAAFDV | 17 |
| heavy chain | | |
| CDR3 for E09 | | |
| SEQ ID NO: 456 | SGNSFNIGRY PVN | 13 |
| light chain | | |
| CDR1 for E09 | | |
| SEQ ID NO: 457 | YNNLRFS | 7 |
| light chain | | |
| CDR2 for E09 | | |
| SEQ ID NO: 458 | STWDDTLKGW V | 11 |
| light chain | | |
| CDR3 for E09 | | |

In an embodiment, the CD95 agonist is an CD95 agonist described in International Patent Application Publication No. WO 2009/007120 A2 and U.S. Patent Application Publication No. US 2016/0176941 A1, the disclosure of each of which is incorporated by reference herein.

In an embodiment, the CD95 agonist is an CD95 agonistic fusion protein as depicted in Structure I-A (C-terminal Fc-antibody fragment fusion protein) or Structure I-B (N-terminal Fc-antibody fragment fusion protein), or a fragment, derivative, conjugate, variant, or biosimilar thereof. The properties of structures I-A and I-B are described above and in U.S. Pat. Nos. 9,359,420, 9,340,599, 8,921,519, and 8,450,460, the disclosures of which are incorporated by reference herein. Amino acid sequences for the polypeptide domains of structure I-A are given in Table 6. The Fc domain preferably comprises a complete constant domain (amino acids 17-230 of SEQ ID NO:31) the complete hinge domain (amino acids 1-16 of SEQ ID NO:31) or a portion of the hinge domain (e.g., amino acids 4-16 of SEQ ID NO:31). Preferred linkers for connecting a C-terminal Fc-antibody may be selected from the embodiments given in SEQ ID NO:33 to SEQ ID NO:41, including linkers suitable for fusion of additional polypeptides. Likewise, amino acid sequences for the polypeptide domains of structure I-B are given in Table 7. If an Fc antibody fragment is fused to the N-terminus of an TNRFSF fusion protein as in structure I-B, the sequence of the Fc module is preferably that shown in SEQ ID NO:42, and the linker sequences are preferably selected from those embodiments set forth in SED ID NO:43 to SEQ ID NO:45.

In an embodiment, an CD95 agonist fusion protein according to structures I-A or I-B comprises one or more CD95 binding domains comprising a CD95 ligand sequence (Table 47). In an embodiment, an CD95 agonist fusion protein according to structures I-A or I-B comprises one or more CD95 binding domains comprising a sequence according to SEQ ID NO:459. In an embodiment, an CD95 agonist fusion protein according to structures I-A or I-B comprises one or more CD95 binding domains comprising a soluble LIGHT sequence. In an embodiment, a CD95 agonist fusion protein according to structures I-A or I-B comprises one or more CD95 binding domains comprising a sequence according to SEQ ID NO:460. In an embodiment, a CD95 agonist fusion protein according to structures I-A or I-B comprises one or more CD95 binding domains comprising a sequence according to SEQ ID NO:461. In an embodiment, a CD95 agonist fusion protein according to structures I-A or I-B comprises one or more CD95 binding domains comprising a sequence according to SEQ ID NO:462.

In an embodiment, an CD95 agonist fusion protein according to structures I-A or I-B comprises one or more CD95 binding domains that is a scFv domain comprising V_{H} and V_{L} regions, wherein the V_{H} and V_{L} domains are connected by a linker.

TABLE 47
Additional polypeptide domains useful as CD95 binding
domains in fusion proteins (e.g., structures I-A and I-B).

| Identifier | Sequence (One-Letter Amino Acid Symbols) | |
|---|---|---|
| SEQ ID NO: 459 | MQQPFNYPYP QIYWVDSSAS SPWAPPGTVL PCPTSVPRRP GQRRPPPPPP PPPLPPPPPP | 60 |
| CD95L (CD95 | PPLPPLPLPP LKKRGNHSTG LCLLVMFFMV LVALVGLGLG MFQLFHLQKE LAELRESTSQ | 120 |
| ligand) | MHTASSLEKQ IGHPSPPPEK KELRKVAHLT GKSNSRSMPL EWEDTYGIVL LSGVKYKKGG | 180 |
| | LVINETGLYF VYSKVYFRGQ SCNNLPLSHK VYMRNSKYPQ DLVMMEGKMM SYCTTGQMWA | 240 |
| | RSSYLGAVFN LTSADHLYVN VSELSLVNFE ESQTFFGLYK L | 281 |
| SEQ ID NO: 460 | VAHLTGKSNS RSMPLEWEDT YGIVLLSGVK YKKGGLVINE TGLYFVYSKV YFRGQSCNNL | 60 |
| CD95L soluble | PLSHKVYMRN SKYPQDLVMM EGKMMSYCTT GQMWARSSYL GAVFNLTSAD HLYVNVSELS | 120 |
| domain | LVNFEESQTF FGLYKL | 136 |
| SEQ ID NO: 461 | AHLTGKSNSR SMPLEWEDTY GIVLLSGVKY KKGGLVINET GLYFVYSKVY FRGQSCNNLP | 60 |
| CD95L soluble | LSHKVYMRNS KYPQDLVMME GKMMSYCTTG QMWARSSYLG AVFNLTSADH LYVNVSELSL | 120 |
| domain | VNFEESQTFF GLYKL | 135 |
| (alternative) | | |
| SEQ ID NO: 462 | HLTGKSNSRS MPLEWEDTYG IVLLSGVKYK KGGLVINETG LYFVYSKVYF RGQSCNNLPL | 60 |
| CD95L soluble | SHKVYMRNSK YPQDLVMMEG KMMSYCTTGQ MWARSSYLGA VFNLTSADHL YVNVSELSLV | 120 |
| domain | NFEESQTFFG LYKL | 134 |
| (alternative) | | |

In an embodiment, the CD95 agonist is a CD95 agonistic single-chain fusion polypeptide comprising (i) a first soluble CD95 binding domain, (ii) a first peptide linker, (iii) a second soluble CD95 binding domain, (iv) a second peptide linker, and (v) a third soluble CD95 binding domain, further comprising an additional domain at the N-terminal and/or C-terminal end, and wherein the additional domain is a Fab or Fc fragment domain. In an embodiment, the CD95 agonist is a CD95 agonistic single-chain fusion polypeptide comprising (i) a first soluble CD95 binding domain, (ii) a first peptide linker, (iii) a second soluble CD95 binding domain, (iv) a second peptide linker, and (v) a third soluble CD95 binding domain, further comprising an additional domain at the N-terminal and/or C-terminal end, wherein the additional domain is a Fab or Fc fragment domain wherein each of the soluble CD95 binding domains lacks a stalk region (which contributes to trimerisation and provides a certain distance to the cell membrane, but is not part of the CD95 binding domain) and the first and the second peptide linkers independently have a length of 3-8 amino acids.

In an embodiment, the CD95 agonist is an CD95 agonistic single-chain fusion polypeptide comprising (i) a first soluble tumor necrosis factor (TNF) superfamily cytokine domain, (ii) a first peptide linker, (iii) a second soluble TNF superfamily cytokine domain, (iv) a second peptide linker, and (v) a third soluble TNF superfamily cytokine domain, wherein each of the soluble TNF superfamily cytokine domains lacks a stalk region and the first and the second peptide linkers independently have a length of 3-8 amino acids, and wherein the TNF superfamily cytokine domain is an CD95 binding domain.

In an embodiment, the CD95 agonist is a CD95 agonistic scFv antibody comprising any of the foregoing V_{H} domains linked to any of the foregoing V_{L} domains.

### Methods of Expanding Tumor Infiltrating Lymphocytes

In an embodiment, the invention provides a method of expanding a population of TILs using any of the TNFRSF agonists of the present disclosure, the method comprising the steps as described in Jin, et al., J. Immunotherapy 2012, 35, 283-292, the disclosure of which is incorporated by reference herein. For example, the tumor may be placed in enzyme media and mechanically dissociated for approximately 1 minute. The mixture may then be incubated for 30 minutes at 37° C. in 5% CO₂ and then mechanically disrupted again for approximately 1 minute. After incubation for 30 minutes at 37° C. in 5% CO₂, the tumor may be mechanically disrupted a third time for approximately 1 minute. If after the third mechanical disruption, large pieces of tissue are present, 1 or 2 additional mechanical dissociations may be applied to the sample, with or without 30 additional minutes of incubation at 37° C. in 5% CO₂. At the end of the final incubation, if the cell suspension contains a large number of red blood cells or dead cells, a density gradient separation using Ficoll may be performed to remove these cells. TIL cultures were initiated in 24-well plates (Costar 24-well cell culture cluster, flat bottom; Corning Incorporated, Corning, NY), each well may be seeded with 1×10⁶ tumor digest cells or one tumor fragment approximately 1 to 8 mm³ in size in 2 mL of complete medium (CM) with IL-2 (6000 IU/mL; Chiron Corp., Emeryville, CA). CM comprises Roswell Park Memorial Institute (RPMI) 1640 buffer with GlutaMAX, supplemented with 10% human AB serum, 25 mM Hepes, and 10 mg/mL gentamicin. Cultures may be initiated in gas-permeable flasks with a 40 mL capacity and a 10 cm² gas-permeable silicon bottom (G-Rex 10; Wilson Wolf Manufacturing, New Brighton, each flask may be loaded with 10-40×10⁶ viable tumor digest cells or 5-30 tumor fragments in 10-40 mL of CM with IL-2. G-Rex 10 and 24-well plates may be incubated in a humidified incubator at 37° C. in 5% CO₂ and 5 days after culture initiation, half the media may be removed and replaced with fresh CM and IL-2 and after day 5, half the media may be changed every 2-3 days. Rapid expansion protocol (REP) of TILs may be performed using T-175 flasks and gas-permeable bags or gas-permeable G-Rex flasks, as described elsewhere herein, using the TNFRSF agonists of the present disclosure. For REP in T-175 flasks, 1×10⁶ TILs may be suspended in 150 mL of media in each flask. The TIL may be cultured with TNFRSF agonists of the present disclosure at a ratio described herein, in a 1 to 1 mixture of CM and AIM-V medium (50/50 medium), supplemented with 3000 IU/mL of IL-2 and 30 ng/mL of anti-CD3 antibody (OKT-3). The T-175 flasks may be incubated at 37° C. in 5% CO₂. Half the media may be changed on day 5 using 50/50 medium with 3000 IU/mL of IL-2. On day 7, cells from 2 T-175 flasks may be combined in a 3 L bag and 300 mL of AIM-V with 5% human AB serum and 3000 IU/mL of IL-2 may be added to the 300 mL of TIL suspension. The number of cells in each bag may be counted every day or two days, and fresh media may be added to keep the cell count between 0.5 and 2.0×10⁶ cells/mL. For REP in 500 mL capacity flasks with 100 cm² gas-permeable silicon bottoms (e.g., G-Rex 100, Wilson Wolf Manufacturing, as described elsewhere herein), 5×10⁶ or 10×10⁶ TILs may be cultured with TNFRSF agonists at a ratio described herein (e.g., 1 to 100) in 400 mL of 50/50 medium, supplemented with 3000 IU/mL of IL-2 and 30 ng/mL of anti-CD3 antibody (OKT-3). The G-Rex100 flasks may be incubated at 37° C. in 5% CO₂. On day five, 250 mL of supernatant may be removed and placed into centrifuge bottles and centrifuged at 1500 rpm (491 g) for 10 minutes. The obtained TIL pellets may be resuspended with 150 mL of fresh 50/50 medium with 3000 IU/mL of IL-2 and added back to the G-Rex 100 flasks. When TIL are expanded serially in G-Rex 100 flasks, on day seven the TIL in each G-Rex100 are suspended in the 300 mL of media present in each flask and the cell suspension may be divided into three 100 mL aliquots that may be used to seed 3 G-Rex100 flasks. About 150 mL of AIM-V with 5% human AB serum and 3000 IU/mL of IL-2 may then be added to each flask. G-Rex100 flasks may then be incubated at 37° C. in 5% CO₂, and after four days, 150 mL of AIM-V with 3000 IU/mL of IL-2 may be added to each G-Rex100 flask. After this, the REP may be completed by harvesting cells on day 14 of culture.

In an embodiment, a method or process of expanding or treating a cancer includes a step wherein TILs are obtained from a patient tumor sample. A patient tumor sample may be obtained using methods known in the art. For example, TILs may be cultured from enzymatic tumor digests and tumor fragments (about 1 to about 8 mm³ in size) from sharp dissection. Such tumor digests may be produced by incubation in enzymatic media (e.g., Roswell Park Memorial Institute (RPMI) 1640 buffer, 2 mM glutamate, 10 mcg/mL gentamicine, 30 units/mL of DNase and 1.0 mg/mL of collagenase) followed by mechanical dissociation (e.g., using a tissue dissociator). Tumor digests may be produced by placing the tumor in enzymatic media and mechanically dissociating the tumor for approximately 1 minute, followed by incubation for 30 minutes at 37° C. in 5% CO₂, followed by repeated cycles of mechanical dissociation and incubation under the foregoing conditions until only small tissue pieces are present. At the end of this process, if the cell suspension contains a large number of red blood cells or dead cells, a density gradient separation using FICOLL branched hydrophilic polysaccharide may be performed to remove these cells. Alternative methods known in the art may be used, such as those described in U.S. Patent Application Publication No. 2012/0244133 A1, the disclosure of which is incorporated by reference herein. Any of the foregoing methods may be used in any of the embodiments described herein for methods or processes of expanding TILs or methods treating a cancer.

In an embodiment, a rapid expansion process for TILs may be performed using T-175 flasks and gas permeable bags as previously described (Tran, et al., J. Immunother. 2008, 31, 742-51; Dudley, et al., J. Immunother. 2003, 26, 332-42) or gas permeable cultureware (G-Rex flasks, commercially available from Wilson Wolf Manufacturing Corporation, New Brighton, MN, USA). For TIL rapid expansion in T-175 flasks, 1×10⁶ TILs suspended in 150 mL of media may be added to each T-175 flask. The TILs may be cultured with TNFRSF agonists at a ratio of 1 TIL to 100 TNFRSF agonists and the cells were cultured in a 1 to 1 mixture of CM and AIM-V medium, supplemented with 3000 IU (international units) per mL of IL-2 and 30 ng per ml of anti-CD3 antibody (e.g., OKT-3). The T-175 flasks may be incubated at 37° C. in 5% CO₂. Half the media may be exchanged on day 5 using 50/50 medium with 3000 IU per mL of IL-2. On day 7 cells from two T-175 flasks may be combined in a 3 L bag and 300 mL of AIM V with 5% human AB serum and 3000 IU per mL of IL-2 was added to the 300 ml of TIL suspension. The number of cells in each bag was counted every day or two and fresh media was added to keep the cell count between 0.5 and 2.0×10⁶ cells/mL.

In an embodiment, for TIL rapid expansions in 500 mL capacity gas permeable flasks with 100 cm² gas-permeable silicon bottoms (G-Rex 100, commercially available from Wilson Wolf Manufacturing Corporation, New Brighton, MN, USA), 5×10⁶ or 10×10⁶ TIL may be cultured with TNFRSF agonists in 400 mL of 50/50 medium, supplemented with 5% human AB serum, 3000 IU per mL of IL-2 and 30 ng per mL of anti-CD3 (OKT-3). The G-Rex 100 flasks may be incubated at 37° C. in 5% CO₂. On day 5, 250 mL of supernatant may be removed and placed into centrifuge bottles and centrifuged at 1500 rpm (revolutions per minute; 491×g) for 10 minutes. The TIL pellets may be re-suspended with 150 mL of fresh medium with 5% human AB serum, 3000 IU per mL of IL-2, and added back to the original G-Rex 100 flasks. When TIL are expanded serially in G-Rex 100 flasks, on day 7 the TIL in each G-Rex 100 may be suspended in the 300 mL of media present in each flask and the cell suspension may be divided into 3 100 mL aliquots that may be used to seed 3 G-Rex 100 flasks. Then 150 mL of AIM-V with 5% human AB serum and 3000 IU per mL of IL-2 may be added to each flask. The G-Rex 100 flasks may be incubated at 37° C. in 5% CO₂ and after 4 days 150 mL of AIM-V with 3000 IU per mL of IL-2 may be added to each G-Rex 100 flask. The cells may be harvested on day 14 of culture.

In an embodiment, TILs may be prepared as follows. 2 mm³ tumor fragments are cultured in complete media (CM) comprised of AIM-V medium (Invitrogen Life Technologies, Carlsbad, CA) supplemented with 2 mM glutamine (Mediatech, Inc. Manassas, VA), 100 U/mL penicillin (Invitrogen Life Technologies), 100 μg/mL streptomycin (Invitrogen Life Technologies), 5% heat-inactivated human AB serum (Valley Biomedical, Inc. Winchester, VA) and 600 IU/mL rhIL-2 (Chiron, Emeryville, CA). For enzymatic digestion of solid tumors, tumor specimens are diced into RPMI-1640, washed and centrifuged at 800 rpm for 5 minutes at 15-22° C., and resuspended in enzymatic digestion buffer (0.2 mg/mL Collagenase and 30 units/ml of DNase in RPMI-1640) followed by overnight rotation at room temperature. TILs established from fragments may be grown for 3-4 weeks in CM and expanded fresh or cryopreserved in heat-inactivated HAB serum with 10% dimethylsulfoxide (DMSO) and stored at −180° C. until the time of study. Tumor associated lymphocytes (TAL) obtained from ascites collections were seeded at 3×10⁶ cells/well of a 24 well plate in CM. TIL growth was inspected about every other day using a low-power inverted microscope.

In an embodiment, the invention includes a method of expanding tumor infiltrating lymphocytes (TILs), the method comprising contacting a population of TILs comprising at least one TIL with a TNFRSF agonist described herein, wherein said TNFRSF agonist comprises at least one co-stimulatory ligand that specifically binds with a co-stimulatory molecule expressed on the cellular surface of the TILs, wherein binding of said co-stimulatory molecule with said co-stimulatory ligand induces proliferation of the TILs, thereby specifically expanding TILs.

In an embodiment, the invention provides a method of expanding a population of tumor infiltrating lymphocytes (TILs), the method comprising the steps of contacting the population of TILs with one or more TNFRSF agonists in a cell culture medium.

In an embodiment, the invention provides a method of expanding a population of tumor infiltrating lymphocytes (TILs), the method comprising the steps of contacting the population of TILs with one or more TNFRSF agonists in a cell culture medium, wherein the concentrations of the one or more TNFRSF agonists in the cell culture medium are independently selected from the group consisting of 50 ng/mL, 100 ng/mL, 500 ng/mL, 1 μg/mL, 5 μg/mL, 10 μg/mL, 20 μg/mL, 30 μg/mL, 40 μg/mL, 50 μg/mL, 60 μg/mL, 70 μg/mL, 80 μg/mL, 90 μg/mL, and 100 μg/mL.

In an embodiment, the invention provides a method of expanding a population of tumor infiltrating lymphocytes (TILs), the method comprising the steps of contacting the population of TILs with one or more TNFRSF agonists in a cell culture medium, wherein the cell culture medium further comprises IL-2 at an initial concentration of about 3000 IU/mL and OKT-3 antibody at an initial concentration of about 30 ng/mL.

In an embodiment, the invention provides a method of expanding a population of tumor infiltrating lymphocytes (TILs), the method comprising the steps of contacting the population of TILs with one or more TNFRSF agonists in a cell culture medium, wherein the cell culture medium further comprises IL-2 at an initial concentration of about 3000 IU/mL and OKT-3 antibody at an initial concentration of about 30 ng/mL, and wherein the one or more TNFRSF agonists comprises a 4-1BB agonist.

In an embodiment, the invention provides a method of expanding a population of tumor infiltrating lymphocytes (TILs), the method comprising the steps of contacting the population of TILs with one or more TNFRSF agonists in a cell culture medium, wherein the cell culture medium further comprises IL-2 at an initial concentration of about 3000 IU/mL and OKT-3 antibody at an initial concentration of about 30 ng/mL, and wherein the one or more TNFRSF agonists comprises an OX40 agonist.

In an embodiment, the invention provides a method of expanding a population of tumor infiltrating lymphocytes (TILs), the method comprising the steps of contacting the population of TILs with one or more TNFRSF agonists in a cell culture medium, wherein the cell culture medium further comprises IL-2 at an initial concentration of about 3000 IU/mL and OKT-3 antibody at an initial concentration of about 30 ng/mL, and wherein the one or more TNFRSF agonists comprises a 4-1BB and an OX40 agonist.

In an embodiment, the invention provides a method of expanding a population of tumor infiltrating lymphocytes (TILs), the method comprising the steps of contacting the population of TILs with one or more TNFRSF agonists in a cell culture medium, wherein the cell culture medium further comprises IL-2 at an initial concentration of about 3000 IU/mL and OKT-3 antibody at an initial concentration of about 30 ng/mL, and wherein the one or more TNFRSF agonists comprises a CD27 agonist.

In an embodiment, the invention provides a method of expanding a population of tumor infiltrating lymphocytes (TILs), the method comprising the steps of contacting the population of TILs with one or more TNFRSF agonists in a cell culture medium, wherein the cell culture medium further comprises IL-2 at an initial concentration of about 3000 IU/mL and OKT-3 antibody at an initial concentration of about 30 ng/mL, and wherein the one or more TNFRSF agonists comprises a GITR agonist.

In an embodiment, the invention provides a method of expanding a population of tumor infiltrating lymphocytes (TILs), the method comprising the steps of contacting the population of TILs with one or more TNFRSF agonists in a cell culture medium, wherein the cell culture medium further comprises IL-2 at an initial concentration of about 3000 IU/mL and OKT-3 antibody at an initial concentration of about 30 ng/mL, and wherein the one or more TNFRSF agonists comprises a HVEM agonist.

In an embodiment, the invention provides a method of expanding a population of tumor infiltrating lymphocytes (TILs), the method comprising the steps of contacting the population of TILs with one or more TNFRSF agonists in a cell culture medium, wherein the cell culture medium further comprises IL-2 at an initial concentration of about 3000 IU/mL and OKT-3 antibody at an initial concentration of about 30 ng/mL, and wherein the one or more TNFRSF agonists comprises a CD95 agonist.

In an embodiment, the invention provides a method of expanding a population of tumor infiltrating lymphocytes (TILs), the method comprising the steps of contacting the population of TILs with one or more TNFRSF agonists in a cell culture medium, wherein the the population of TILs by at least 50-fold over a period of 7 days in the cell culture medium.

In an embodiment, the invention provides a method of expanding a population of tumor infiltrating lymphocytes (TILs), the method comprising the steps of contacting the population of TILs with one or more TNFRSF agonists in a cell culture medium, wherein the the population of TILs by at least 50-fold over a period of 7 days in the cell culture medium, and wherein the expansion is performed using a gas permeable container.

In an embodiment, REP can be performed in a gas permeable container using the TNFRSF agonists of the present disclosure by any suitable method. For example, TILs can be rapidly expanded using non-specific T-cell receptor stimulation in the presence of interleukin-2 (IL-2) or interleukin-15 (IL-15). The non-specific T-cell receptor stimulus can include, for example, an anti-CD3 antibody, such as about 30 ng/mL of OKT-3, a monoclonal anti-CD3 antibody (commercially available from Ortho-McNeil, Raritan, NJ or Miltenyi Biotech, Auburn, CA) or UHCT-1 (commercially available from BioLegend, San Diego, CA, USA). TILs can be rapidly expanded by further stimulation of the TILs in vitro with one or more antigens, including antigenic portions thereof, such as epitope(s), of the cancer, which can be optionally expressed from a vector, such as a human leukocyte antigen A2 (HLA-A2) binding peptide, e.g., 0.3 μM MART-1:26-35 (27 L) or gpl 00:209-217 (210M), optionally in the presence of a T-cell growth factor, such as 300 IU/mL IL-2 or IL-15. Other suitable antigens may include, e.g., NY-ESO-1, TRP-1, TRP-2, tyrosinase cancer antigen, MAGE-A3, SSX-2, and VEGFR2, or antigenic portions thereof. TIL may also be rapidly expanded by re-stimulation with the same antigen(s) of the cancer pulsed onto HLA-A2-expressing antigen-presenting cells. Alternatively, the TILs can be further re-stimulated with, e.g., example, irradiated, autologous lymphocytes or with irradiated HLA-A2+ allogeneic lymphocytes and IL-2.

In an embodiment, a method for expanding TILs may include using about 5000 mL to about 25000 mL of cell culture medium, about 5000 mL to about 10000 mL of cell culture medium, or about 5800 mL to about 8700 mL of cell culture medium. In an embodiment, a method for expanding TILs may include using about 1000 mL to about 2000 mL of cell medium, about 2000 mL to about 3000 mL of cell culture medium, about 3000 mL to about 4000 mL of cell culture medium, about 4000 mL to about 5000 mL of cell culture medium, about 5000 mL to about 6000 mL of cell culture medium, about 6000 mL to about 7000 mL of cell culture medium, about 7000 mL to about 8000 mL of cell culture medium, about 8000 mL to about 9000 mL of cell culture medium, about 9000 mL to about 10000 mL of cell culture medium, about 10000 mL to about 15000 mL of cell culture medium, about 15000 mL to about 20000 mL of cell culture medium, or about 20000 mL to about 25000 mL of cell culture medium. In an embodiment, expanding the number of TILs uses no more than one type of cell culture medium. Any suitable cell culture medium may be used, e.g., AIM-V cell medium (L-glutamine, 50 μM streptomycin sulfate, and 10 μM gentamicin sulfate) cell culture medium (Invitrogen, Carlsbad CA). In this regard, the inventive methods advantageously reduce the amount of medium and the number of types of medium required to expand the number of TIL. In an embodiment, expanding the number of TIL may comprise feeding the cells no more frequently than every third or fourth day. Expanding the number of cells in a gas permeable container simplifies the procedures necessary to expand the number of cells by reducing the feeding frequency necessary to expand the cells.

In an embodiment, the rapid expansion is performed using a gas permeable container. Such embodiments allow for cell populations to expand from about 5×10⁵ cells/cm² to between 10×10⁶ and 30×10⁶ cells/cm². In an embodiment, this expansion occurs without feeding. In an embodiment, this expansion occurs without feeding so long as medium resides at a height of about 10 cm in a gas-permeable flask. In an embodiment this is without feeding but with the addition of one or more cytokines. In an embodiment, the cytokine can be added as a bolus without any need to mix the cytokine with the medium. Such containers, devices, and methods are known in the art and have been used to expand TILs, and include those described in U.S. Patent Application Publication No. US 2014/0377739 A1, International Patent Application Publication No. WO 2014/210036 A1, U.S. Patent Application Publication No. US 2013/0115617 A1, International Publication No. WO 2013/188427 A1, U.S. Patent Application Publication No. US 2011/0136228 A1, U.S. Pat. No. 8,809,050, International Patent Application Publication No. WO 2011/072088 A2, U.S. Patent Application Publication No. US 2016/0208216 A1, U.S. Patent Application Publication No. US 2012/0244133 A1, International Patent Application Publication No. WO 2012/129201 A1, U.S. Patent Application Publication No. US 2013/0102075 A1, U.S. Pat. No. 8,956,860, International Patent Application Publication No. WO 2013/173835 A1, and U.S. Patent Application Publication No. US 2015/0175966 A1, the disclosures of which are incorporated herein by reference. Such processes are also described in Jin, et al., J. Immunotherapy 2012, 35, 283-292, the disclosure of which is incorporated by reference herein.

In an embodiment, the gas permeable container is a G-Rex 10 flask (Wilson Wolf Manufacturing Corporation, New Brighton, MN, USA). In an embodiment, the gas permeable container includes a 10 cm² gas permeable culture surface. In an embodiment, the gas permeable container includes a 40 mL cell culture medium capacity. In an embodiment, the gas permeable container provides 100 to 300 million TILs after 2 medium exchanges.

In an embodiment, the gas permeable container is a G-Rex 100 flask (Wilson Wolf Manufacturing Corporation, New Brighton, MN, USA). In an embodiment, the gas permeable container includes a 100 cm² gas permeable culture surface. In an embodiment, the gas permeable container includes a 450 mL cell culture medium capacity. In an embodiment, the gas permeable container provides 1 to 3 billion TILs after 2 medium exchanges.

In an embodiment, the gas permeable container is a G-Rex 100M flask (Wilson Wolf Manufacturing Corporation, New Brighton, MN, USA). In an embodiment, the gas permeable container includes a 100 cm² gas permeable culture surface. In an embodiment, the gas permeable container includes a 1000 mL cell culture medium capacity. In an embodiment, the gas permeable container provides 1 to 3 billion TILs without medium exchange.

In an embodiment, the gas permeable container is a G-Rex 100 L flask (Wilson Wolf Manufacturing Corporation, New Brighton, MN, USA). In an embodiment, the gas permeable container includes a 100 cm² gas permeable culture surface. In an embodiment, the gas permeable container includes a 2000 mL cell culture medium capacity. In an embodiment, the gas permeable container provides 1 to 3 billion TILs without medium exchange.

In an embodiment, the gas permeable container is a G-Rex 24 well plate (Wilson Wolf Manufacturing Corporation, New Brighton, MN, USA). In an embodiment, the gas permeable container includes a plate with wells, wherein each well includes a 2 cm² gas permeable culture surface. In an embodiment, the gas permeable container includes a plate with wells, wherein each well includes an 8 mL cell culture medium capacity. In an embodiment, the gas permeable container provides 20 to 60 million cells per well after 2 medium exchanges.

In an embodiment, the gas permeable container is a G-Rex 6 well plate (Wilson Wolf Manufacturing Corporation, New Brighton, MN, USA). In an embodiment, the gas permeable container includes a plate with wells, wherein each well includes a 10 cm² gas permeable culture surface. In an embodiment, the gas permeable container includes a plate with wells, wherein each well includes a 40 mL cell culture medium capacity. In an embodiment, the gas permeable container provides 100 to 300 million cells per well after 2 medium exchanges.

In an embodiment, the cell medium in the first and/or second gas permeable container is unfiltered. The use of unfiltered cell medium may simplify the procedures necessary to expand the number of cells. In an embodiment, the cell medium in the first and/or second gas permeable container lacks beta-mercaptoethanol (BME).

In an embodiment, the duration of the method comprising obtaining a tumor tissue sample from the mammal; culturing the tumor tissue sample in a first gas permeable container containing cell medium therein; obtaining TILs from the tumor tissue sample; expanding the number of TILs in a second gas permeable container containing cell medium therein using TNFRSF agonists for a duration of about 14 to about 42 days, e.g., about 28 days.

In an embodiment, the ratio of TILs to TNFRSF agonists (cells to moles) in the rapid expansion is about 1 to 25, about 1 to 50, about 1 to 100, about 1 to 125, about 1 to 150, about 1 to 175, about 1 to 200, about 1 to 225, about 1 to 250, about 1 to 275, about 1 to 300, about 1 to 325, about 1 to 350, about 1 to 500, about 1 to 1000, or about 1 to 10000. In an embodiment, the ratio of TILs to TNFRSF agonists in the rapid expansion is between 1 to 50 and 1 to 300. In an embodiment, the ratio of TILs to TNFRSF agonists in the rapid expansion is between 1 to 100 and 1 to 200.

In an embodiment, the ratio of TILs to TNFRSF agonist (TIL:TNFRSF agonist, cells to moles) is selected from the group consisting of 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:225, 1:250, 1:275, 1:300, 1:350, 1:400, 1:450, 1:500, 1:1000, 1:5000, 1:10000, and 1:50000.

In an embodiment, TILs are expanded in gas-permeable containers. Gas-permeable containers have been used to expand TILs using PBMCs using methods, compositions, and devices known in the art, including those described in U.S. Patent Application Publication No. U.S. Patent Application Publication No. 2005/0106717 A1, the disclosures of which are incorporated herein by reference. In an embodiment, TILs are expanded in gas-permeable bags. In an embodiment, TILs are expanded using a cell expansion system that expands TILs in gas permeable bags, such as the Xuri Cell Expansion System W25 (GE Healthcare). In an embodiment, TILs are expanded using a cell expansion system that expands TILs in gas permeable bags, such as the WAVE Bioreactor System, also known as the Xuri Cell Expansion System W5 (GE Healthcare). In an embodiment, the cell expansion system includes a gas permeable cell bag with a volume selected from the group consisting of about 100 mL, about 200 mL, about 300 mL, about 400 mL, about 500 mL, about 600 mL, about 700 mL, about 800 mL, about 900 mL, about 1 L, about 2 L, about 3 L, about 4 L, about 5 L, about 6 L, about 7 L, about 8 L, about 9 L, about 10 L, about 11 L, about 12 L, about 13 L, about 14 L, about 15 L, about 16 L, about 17 L, about 18 L, about 19 L, about 20 L, about 25 L, and about 30 L. In an embodiment, the cell expansion system includes a gas permeable cell bag with a volume range selected from the group consisting of between 50 and 150 mL, between 150 and 250 mL, between 250 and 350 mL, between 350 and 450 mL, between 450 and 550 mL, between 550 and 650 mL, between 650 and 750 mL, between 750 and 850 mL, between 850 and 950 mL, and between 950 and 1050 mL. In an embodiment, the cell expansion system includes a gas permeable cell bag with a volume range selected from the group consisting of between 1 L and 2 L, between 2 L and 3 L, between 3 L and 4 L, between 4 L and 5 L, between 5 L and 6 L, between 6 L and 7 L, between 7 L and 8 L, between 8 L and 9 L, between 9 L and 10 L, between 10 L and 11 L, between 11 L and 12 L, between 12 L and 13 L, between 13 L and 14 L, between 14 L and 15 L, between 15 L and 16 L, between 16 L and 17 L, between 17 L and 18 L, between 18 L and 19 L, and between 19 L and 20 L. In an embodiment, the cell expansion system includes a gas permeable cell bag with a volume range selected from the group consisting of between 0.5 L and 5 L, between 5 L and 10 L, between 10 L and 15 L, between 15 L and 20 L, between 20 L and 25 L, and between 25 L and 30 L. In an embodiment, the cell expansion system utilizes a rocking time of about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, and about 28 days. In an embodiment, the cell expansion system utilizes a rocking time of between 30 minutes and 1 hour, between 1 hour and 12 hours, between 12 hours and 1 day, between 1 day and 7 days, between 7 days and 14 days, between 14 days and 21 days, and between 21 days and 28 days. In an embodiment, the cell expansion system utilizes a rocking rate of about 2 rocks/minute, about 5 rocks/minute, about 10 rocks/minute, about 20 rocks/minute, about 30 rocks/minute, and about 40 rocks/minute. In an embodiment, the cell expansion system utilizes a rocking rate of between 2 rocks/minute and 5 rocks/minute, 5 rocks/minute and 10 rocks/minute, 10 rocks/minute and 20 rocks/minute, 20 rocks/minute and 30 rocks/minute, and 30 rocks/minute and 40 rocks/minute. In an embodiment, the cell expansion system utilizes a rocking angle of about 2°, about 3°, about 4°, about 5°, about 6°, about 7°, about 8°, about 9°, about 10°, about 11°, and about 12°. In an embodiment, the cell expansion system utilizes a rocking angle of between 2° and 3°, between 3° and 4°, between 4° and 5°, between 5° and 6°, between 6° and 7°, between 7° and 8°, between 8° and 9°, between 9° and 10°, between 10° and 11°, and between 11° and 12°.

In an embodiment, a method of expanding TILs using TNFRSF agonists further comprises a step wherein TILs are selected for superior tumor reactivity. Any selection method known in the art may be used. For example, the methods described in U.S. Patent Application Publication No. 2016/0010058 A1, the disclosures of which are incorporated herein by reference, may be used for selection of TILs for superior tumor reactivity.

In an embodiment, the cell culture medium further comprises OKT-3 antibody. In a preferred embodiment, the cell culture medium comprises about 30 ng/mL of OKT-3 antibody. In an embodiment, the cell culture medium comprises about 0.1 ng/mL, about 0.5 ng/mL, about 1 ng/mL, about 2.5 ng/mL, about 5 ng/mL, about 7.5 ng/mL, about 10 ng/mL, about 15 ng/mL, about 20 ng/mL, about 25 ng/mL, about 30 ng/mL, about 35 ng/mL, about 40 ng/mL, about 50 ng/mL, about 60 ng/mL, about 70 ng/mL, about 80 ng/mL, about 90 ng/mL, about 100 ng/mL, about 200 ng/mL, about 500 ng/mL, and about 1 μg/mL of OKT-3 antibody. In an embodiment, the cell culture medium comprises between 0.1 ng/mL and 1 ng/mL, between 1 ng/mL and 5 ng/mL, between 5 ng/mL and 10 ng/mL, between 10 ng/mL and 20 ng/mL, between 20 ng/mL and 30 ng/mL, between 30 ng/mL and 40 ng/mL, between 40 ng/mL and 50 ng/mL, or between 50 ng/mL and 100 ng/mL of OKT-3 antibody. In an embodiment, the cell culture medium comprises between 10 ng/mL and 60 ng/mL of OKT-3 antibody.

In an embodiment, the cell culture medium further comprises IL-2. In a preferred embodiment, the cell culture medium comprises about 3000 IU/mL of IL-2. In an embodiment, the cell culture medium comprises about 500 IU/mL, about 700 IU/mL, about 800 IU/mL, about 1000 IU/mL, about 1100 IU/mL, about 1200 IU/mL, about 1500 IU/mL, about 2000 IU/mL, about 2500 IU/mL, about 3000 IU/mL, about 3500 IU/mL, about 4000 IU/mL, about 4500 IU/mL, about 5000 IU/mL, about 5500 IU/mL, about 6000 IU/mL, about 6500 IU/mL, about 7000 IU/mL, about 7500 IU/mL, or about 8000 IU/mL of IL-2. In an embodiment, the cell culture medium comprises between 500 and 1000 IU/mL, 800 and 1200 IU/mL, 1000 and 2000 IU/mL, between 2000 and 3000 IU/mL, between 3000 and 4000 IU/mL, between 4000 and 5000 IU/mL, between 5000 and 6000 IU/mL, between 6000 and 7000 IU/mL, between 7000 and 8000 IU/mL, or between 8000 IU/mL of IL-2. In an embodiment, the cell culture medium comprises between 10 and 6000 IU/mL of IL-2. In an embodiment, the cell culture medium comprises between 500 and 2000 IU/mL of IL-2. In an embodiment, the cell culture medium comprises between 800 and 1100 IU/mL of IL-2.

In an embodiment, the cell culture medium further comprises IL-15, as described, e.g., in International Patent Application Publication Nos. WO 2015/189356 A1 and WO 2015/189356 A1, the disclosures of each of which are incorporated by reference herein. In an embodiment, the cell culture medium comprises about 0.1 ng/mL, about 0.5 ng/mL, about 1 ng/mL, about 2.5 ng/mL, about 5 ng/mL, about 7.5 ng/mL, about 10 ng/mL, about 15 ng/mL, about 20 ng/mL, about 25 ng/mL, about 30 ng/mL, about 35 ng/mL, about 40 ng/mL, about 50 ng/mL, about 60 ng/mL, about 70 ng/mL, about 80 ng/mL, about 90 ng/mL, about 100 ng/mL, about 200 ng/mL, about 500 ng/mL, or about 1 μg/mL of IL-15. In an embodiment, the cell culture medium comprises between 0.1 ng/mL and 100 ng/mL, between 2 ng/mL and 50 ng/mL, or between 5 ng/mL and 25 ng/mL of IL-15. In an embodiment, the cell culture medium comprises between 10 ng/mL and 20 ng/mL, between 20 ng/mL and 30 ng/mL, between 30 ng/mL and 40 ng/mL, between 40 ng/mL and 50 ng/mL, between 50 ng/mL and 60 ng/mL, between 60 ng/mL and 70 ng/mL, between 70 ng/mL and 80 ng/mL, between 80 ng/mL and 90, ir between 90 ng/mL and 100 ng/mL of IL-15.

In an embodiment, the cell culture medium further comprises IL-21, as described, e.g., in International Patent Application Publication Nos. WO 2015/189356 A1 and WO 2015/189356 A1, the disclosures of each of which are incorporated by reference herein. In an embodiment, the cell culture medium comprises about 0.1 ng/mL, about 0.5 ng/mL, about 1 ng/mL, about 2.5 ng/mL, about 5 ng/mL, about 7.5 ng/mL, about 10 ng/mL, about 15 ng/mL, about 20 ng/mL, about 25 ng/mL, about 30 ng/mL, about 35 ng/mL, about 40 ng/mL, about 50 ng/mL, about 60 ng/mL, about 70 ng/mL, about 80 ng/mL, about 90 ng/mL, about 100 ng/mL, about 200 ng/mL, about 500 ng/mL, or about 1 μg/mL of IL-21. In an embodiment, the cell culture medium comprises between 0.1 ng/mL and 100 ng/mL, between 2 ng/mL and 50 ng/mL, or between 5 ng/mL and 25 ng/mL of IL-21. In an embodiment, the cell culture medium comprises between 10 ng/mL and 20 ng/mL, between 20 ng/mL and 30 ng/mL, between 30 ng/mL and 40 ng/mL, between 40 ng/mL and 50 ng/mL, between 50 ng/mL and 60 ng/mL, between 60 ng/mL and 70 ng/mL, between 70 ng/mL and 80 ng/mL, between 80 ng/mL and 90, ir between 90 ng/mL and 100 ng/mL of IL-21.

In an embodiment, the cell culture medium further comprises IL-4 and/or IL-7.

In an embodiment, the TNFRSF agonists of the present invention may be used to expand T cells. Any of the foregoing embodiments of the present invention described for the expansion of TILs may also be applied to the expansion of T cells. In an embodiment, the TNFRSF agonists of the present invention may be used to expand CD8⁺ T cells. In an embodiment, the TNFRSF agonists of the present invention may be used to expand CD4⁺ T cells. In an embodiment, the TNFRSF agonists of the present invention may be used to expand T cells transduced with a chimeric antigen receptor (CAR-T). In an embodiment, the TNFRSF agonists of the present invention may be used to expand T cells comprising a modified T cell receptor (TCR). The CAR-T cells may be targeted against any suitable antigen, including CD19, as described in the art, e.g., in U.S. Pat. Nos. 7,070,995; 7,446,190; 8,399,645; 8,916,381; and 9,328,156; the disclosures of which are incorporated by reference herein. The modified TCR cells may be targeted against any suitable antigen, including NY-ESO-1, TRP-1, TRP-2, tyrosinase cancer antigen, MAGE-A3, SSX-2, and VEGFR2, or antigenic portions thereof, as described in the art, e.g., in U.S. Pat. Nos. 8,367,804 and 7,569,664, the disclosures of which are incorporated by reference herein.

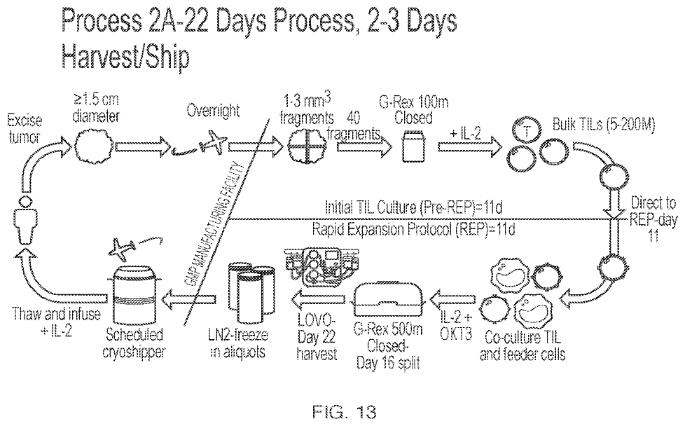

In another embodiment, an exemplary TIL manufacturing/expansion process known as process 2A is schematically illustrated in FIG. 13. In certain aspects, the present methods produce TILs which are capable of increased replication cycles upon administration to a subject/patient and as such may provide additional therapeutic benefits over older TILs (i.e., TILs which have further undergone more rounds of replication prior to administration to a subject/patient). Features of younger TILs have been described in the literature, for example Donia, at al., Scandinavian Journal of Immunology, 75:157-167 (2012); Dudley et al., Clin Cancer Res, 16:6122-6131 (2010); Huang et al., J Immunother, 28(3):258-267 (2005); Besser et al., Clin Cancer Res, 19(17):OF1-OF9 (2013); Besser et al., J Immunother 32:415-423 (2009); Robbins, et al., J Immunol 2004; 173:7125-7130; Shen et al., J Immunother, 30:123-129 (2007); Zhou, et al., J Immunother, 28:53-62 (2005); and Tran, et al., J Immunother, 31:742-751 (2008), all of which are incorporated herein by reference in their entireties.

As discussed herein, the present invention can include a step relating to the restimulation of cyropreserved TILs to increase their metabolic activity and thus relative health prior to transplant into a patient, and methods of testing said metabolic health. As generally outlined herein, TILs are generally taken from a patient sample and manipulated to expand their number prior to transplant into a patient. In some embodiments, the TILs may be optionally genetically manipulated as discussed below.

In some embodiments, the TILs may be cryopreserved. Once thawed, they may also be restimulated to increase their metabolism prior to infusion into a patient.

In some embodiments, the first expansion (including processes referred to as the preREP) is shortened in comparison to conventional expansion methods to 7-14 days and the second expansion (including processes referred to as the REP) is shortened to 7-14 days, as discussed in detail below as well as in the examples and figures.

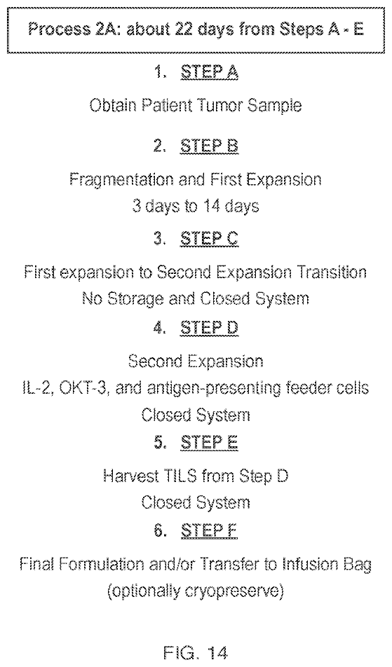

FIG. 14 illustrates an exemplary 2A process. As illustrated in FIG. 14 and further explained in detail below, in some embodiments, the first expansion (Step B) is shortened to 11 days and the second expansion (Step D) is shortened to 11 days. In some embodiments, the combination of the first and second expansions (Step B and Step D) is shortened to 22 days, as discussed in detail below and in the examples and figures. As will be appreciated, the process illustrated in FIG. 14 and described below is exemplary and the methods described herein encompass alterations and additions to the described steps as well as any combinations.

In general, TILs are initially obtained from a patient tumor sample (“primary TILs”) and then expanded into a larger population for further manipulation as described herein, optionally cyropreserved, restimulated as outlined herein and optionally evaluated for phenotype and metabolic parameters as an indication of TIL health.

A patient tumor sample may be obtained using methods known in the art, generally via surgical resection, needle biopsy or other means for obtaining a sample that contains a mixture of tumor and TIL cells. In general, the tumor sample may be from any solid tumor, including primary tumors, invasive tumors or metastatic tumors. The tumor sample may also be a liquid tumor, such as a tumor obtained from a hematological malignancy. The solid tumor may be of any cancer type, including, but not limited to, breast, pancreatic, prostate, colorectal, lung, brain, renal, stomach, and skin (including but not limited to squamous cell carcinoma, basal cell carcinoma, and melanoma). In some embodiments, useful TILs are obtained from malignant melanoma tumors, as these have been reported to have particularly high levels of TILs. In some embodiments, the tumor is greater than about 1.5 cm but less than about 4 cm. In some embodiments, the tumor is less than 4 cm.

Once obtained, the tumor sample is generally fragmented using sharp dissection into small pieces of between 1 to about 8 mm³, with from about 2-3 mm³ being particularly useful. The TILs are cultured from these fragments using enzymatic tumor digests. Such tumor digests may be produced by incubation in enzymatic media (e.g., Roswell Park Memorial Institute (RPMI) 1640 buffer, 2 mM glutamate, 10 mcg/mL gentamicine, 30 units/mL of DNase and 1.0 mg/mL of collagenase) followed by mechanical dissociation (e.g., using a tissue dissociator). Tumor digests may be produced by placing the tumor in enzymatic media and mechanically dissociating the tumor for approximately 1 minute, followed by incubation for 30 minutes at 37° C. in 5% CO₂, followed by repeated cycles of mechanical dissociation and incubation under the foregoing conditions until only small tissue pieces are present. At the end of this process, if the cell suspension contains a large number of red blood cells or dead cells, a density gradient separation using FICOLL branched hydrophilic polysaccharide may be performed to remove these cells. Alternative methods known in the art may be used, such as those described in U.S. Patent Application Publication No. 2012/0244133 A1, the disclosure of which is incorporated by reference herein. Any of the foregoing methods may be used in any of the embodiments described herein for methods and processes of expanding TILs or methods treating a cancer.

In general, the harvested cell suspension is called a “primary cell population” or a “freshly harvested” cell population.

In an embodiment, TILs can be initially cultured from enzymatic tumor digests and tumor fragments obtained from patients.

In some embodiments, the TILs, are obtained from tumor fragments. In some embodiments, the tumor fragment is obtained sharp dissection. In some embodiments, the tumor fragment is between about 1 mm³ and 10 mm³. In some embodiments, the tumor fragment is between about 1 mm³ and 8 mm³. In some embodiments, the tumor fragment is about 1 mm³. In some embodiments, the tumor fragment is about 2 mm³. In some embodiments, the tumor fragment is about 3 mm³. In some embodiments, the tumor fragment is about 4 mm³. In some embodiments, the tumor fragment is about 5 mm³. In some embodiments, the tumor fragment is about 6 mm³. In some embodiments, the tumor fragment is about 7 mm³. In some embodiments, the tumor fragment is about 8 mm³. In some embodiments, the tumor fragment is about 9 mm³. In some embodiments, the tumor fragment is about 10 mm³. In some embodiments, about the tumor fragment is about 8-27 mm³. In some embodiments, about the tumor fragment is about 10-25 mm³. In some embodiments, about the tumor fragment is about 15-25 mm³. In some embodiments, the tumor fragment is about 8-20 mm³. In some embodiments, the tumor fragment is about 15-20 mm³. In some embodiments, the tumor fragment is about 8-15 mm³. In some embodiments, the tumor fragment is about 8-10 mm³.

In some embodiments, the number of tumor fragments is about 40 to about 50 tumor fragments. In some embodiments, the number of tumor fragments is about 40 tumor fragments. In some embodiments, the number of tumor fragments is about 50 tumor fragments. In some embodiments, the tumor fragment size is about 8-27 mm³ and there are less than about 50 tumor fragments.

In some embodiments, the TILs, are obtained from tumor digests. In some embodiments, tumor digests were generated by incubation in enzyme media, for example but not limited to RPMI 1640, 2 mM GlutaMAX, 10 mg/mL gentamicin, 30 U/mL DNase, and 1.0 mg/mL collagenase, followed by mechanical dissociation (GentleMACS, Miltenyi Biotec, Auburn, CA). After placing the tumor in enzyme media, the tumor can be mechanically dissociated for approximately 1 minute. The solution can then be incubated for 30 minutes at 37° C. in 5% CO₂ and it then mechanically disrupted again for approximately 1 minute. After being incubated again for 30 minutes at 37° C. in 5% CO₂, the tumor can be mechanically disrupted a third time for approximately 1 minute. In some embodiments, after the third mechanical disruption if large pieces of tissue were present, 1 or 2 additional mechanical dissociations were applied to the sample, with or without 30 additional minutes of incubation at 37° C. in 5% CO₂. In some embodiments, at the end of the final incubation if the cell suspension contained a large number of red blood cells or dead cells, a density gradient separation using Ficoll can be performed to remove these cells.

After dissection or digestion of tumor fragments in Step A, the resulting cells are cultured in serum containing IL-2 under conditions that favor the growth of TILs over tumor and other cells. In some embodiments, the tumor digests are incubated in 2 mL wells in media comprising inactivated human AB serum with 6000 IU/mL of IL-2. This primary cell population is cultured for a period of days, generally from 3 to 14 days, resulting in a bulk TIL population, generally about 1×10⁸ bulk TIL cells. In some embodiments, this primary cell population is cultured for a period of 7 to 14 days, resulting in a bulk TIL population, generally about 1×10⁸ bulk TIL cells. In some embodiments, this primary cell population is cultured for a period of 10 to 14 days, resulting in a bulk TIL population, generally about 1×10⁸ bulk TIL cells. In some embodiments, this primary cell population is cultured for a period of about 11 days, resulting in a bulk TIL population, generally about 1×10⁸ bulk TIL cells. In some embodiments, this primary cell population is cultured for a period of about 11 days, resulting in a bulk TIL population, generally less than or equal to about 200×10⁶ bulk TIL cells.

In a preferred embodiment, expansion of TILs may be performed using an initial bulk TIL expansion step (Step B as pictured in FIG. 14, which can include processes referred to as pre-REP) as described below and herein, followed by a second expansion (Step D, including processes referred to as rapid expansion protocol (REP) steps) as described below under Step D and herein, followed by optional cryopreservation, and followed by a second Step D (including processes referred to as restimulation REP steps) as described below and herein. The TILs obtained from this process may be optionally characterized for phenotypic characteristics and metabolic parameters as described herein.

In embodiments where TIL cultures are initiated in 24-well plates, for example, using Costar 24-well cell culture cluster, flat bottom (Corning Incorporated, Corning, NY, each well can be seeded with 1×10⁶ tumor digest cells or one tumor fragment in 2 mL of complete medium (CM) with IL-2 (6000 IU/mL; Chiron Corp., Emeryville, CA). In some embodiments, the tumor fragment is between about 1 mm³ and 10 mm³.

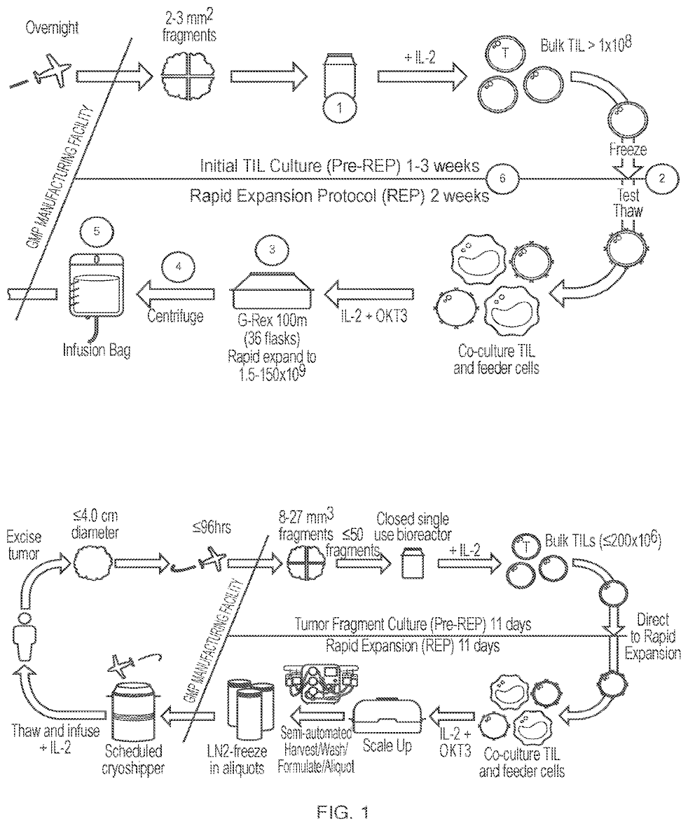

In some embodiments, CM for Step B consists of RPMI 1640 with GlutaMAX, supplemented with 10% human AB serum, 25 mM HEPES, and 10 mg/mL gentamicin. In embodiments where cultures are initiated in gas-permeable flasks with a 40 mL capacity and a 10 cm² gas-permeable silicon bottom (for example, G-Rex10; Wilson Wolf Manufacturing, New Brighton, MN) (FIG. 1), each flask was loaded with 10-40×10⁶ viable tumor digest cells or 5-30 tumor fragments in 10-40 mL of CM with IL-2. Both the G-Rex10 and 24-well plates were incubated in a humidified incubator at 37° C. in 5% CO₂ and 5 days after culture initiation, half the media was removed and replaced with fresh CM and IL-2 and after day 5, half the media was changed every 2-3 days.

In an embodiment, the cell culture medium further comprises IL-2. In a preferred embodiment, the cell culture medium comprises about 3000 IU/mL of IL-2. In an embodiment, the cell culture medium comprises about 1000 IU/mL, about 1500 IU/mL, about 2000 IU/mL, about 2500 IU/mL, about 3000 IU/mL, about 3500 IU/mL, about 4000 IU/mL, about 4500 IU/mL, about 5000 IU/mL, about 5500 IU/mL, about 6000 IU/mL, about 6500 IU/mL, about 7000 IU/mL, about 7500 IU/mL, or about 8000 IU/mL of IL-2. In an embodiment, the cell culture medium comprises between 1000 and 2000 IU/mL, between 2000 and 3000 IU/mL, between 3000 and 4000 IU/mL, between 4000 and 5000 IU/mL, between 5000 and 6000 IU/mL, between 6000 and 7000 IU/mL, between 7000 and 8000 IU/mL, or between 8000 IU/mL of IL-2.

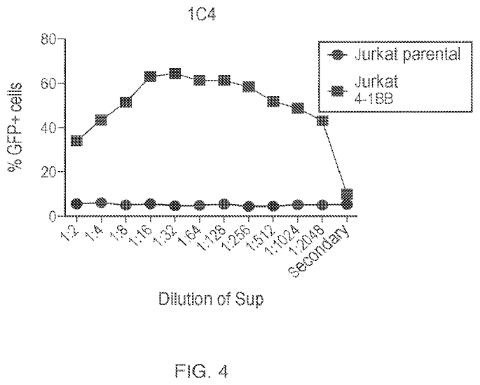

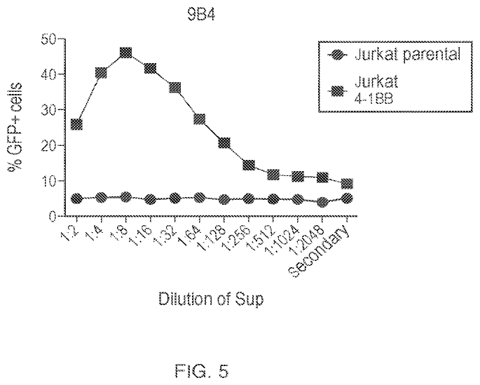

In some embodiments, the first expansion (including processes referred to as the pre-REP; Step B) process is shortened to 3-14 days, as discussed in the examples and figures. In some embodiments, the first expansion of Step B is shortened to 7-14 days, as discussed in the Examples and shown in FIGS. 4 and 5. In some embodiments, the first expansion of Step B is shortened to 10-14 days, as discussed in the Examples and shown in FIGS. 4 and 5. In some embodiments, the first expansion of Step B is shortened to 11 days, as discussed in the Examples and shown in FIGS. 4 and 5.

In some embodiments, IL-2, IL-7, IL-15, and IL-21 as well as combinations thereof can be included during Step B processes as described herein.

In some embodiments, Step B is performed in a closed system bioreactor. In some embodiments, a closed system is employed for the TIL expansion, as described herein. In some embodiments, a single bioreactor is employed. In some embodiments, the single bioreactor employed is for example a GREX-10 or a GREX-100.

In some embodiments, the bulk TIL population from Step B can be cryopreserved immediately, using methods known in the art and described herein. Alternatively, the bulk TIL population can be subjected to a second expansion (REP) and then cryopreserved as discussed below.

In some embodiments, the Step B TILs are not stored and the Step B TILs proceed directly to Step D. In some embodiments, the transition occurs in a closed system, as further described herein.

In some embodiments, the TIL cell population is expanded in number after harvest and initial bulk processing (i.e., after Step A and Step B). This is referred to herein as the second expansion, which can include expansion processes generally referred to in the art as a rapid expansion process (REP). The second expansion is generally accomplished using culture media comprising a number of components, including feeder cells, a cytokine source, and an anti-CD3 antibody, in a gas-permeable container. In some embodiments, the second expansion can include scaling-up in order to increase the number of TILs obtained in the second expansion.

In an embodiment, REP and/or the second expansion can be performed in a gas permeable container using the methods of the present disclosure. For example, TILs can be rapidly expanded using non-specific T-cell receptor stimulation in the presence of interleukin-2 (IL-2) or interleukin-15 (IL-15). The non-specific T-cell receptor stimulus can include, for example, about 30 ng/ml of OKT3, a mouse monoclonal anti-CD3 antibody (commercially available from Ortho-McNeil, Raritan, NJ or Miltenyi Biotech, Auburn, CA). TILs can be rapidly expanded further stimulation of the TILs in vitro with one or more antigens, including antigenic portions thereof, such as epitope(s), of the cancer, which can be optionally expressed from a vector, such as a human leukocyte antigen A2 (HLA-A2) binding peptide, e.g., 0.3 μM MART-1:26-35 (27 L) or gpl 00:209-217 (210M), optionally in the presence of a T-cell growth factor, such as 300 IU/mL IL-2 or IL-15. Other suitable antigens may include, e.g., NY-ESO-1, TRP-1, TRP-2, tyrosinase cancer antigen, MAGE-A3, SSX-2, and VEGFR2, or antigenic portions thereof. TIL may also be rapidly expanded by re-stimulation with the same antigen(s) of the cancer pulsed onto HLA-A2-expressing antigen-presenting cells. Alternatively, the TILs can be further re-stimulated with, e.g., example, irradiated, autologous lymphocytes or with irradiated HLA-A2+ allogeneic lymphocytes and IL-2.

In an embodiment, the cell culture medium further comprises IL-2. In a preferred embodiment, the cell culture medium comprises about 3000 IU/mL of IL-2. In an embodiment, the cell culture medium comprises about 1000 IU/mL, about 1500 IU/mL, about 2000 IU/mL, about 2500 IU/mL, about 3000 IU/mL, about 3500 IU/mL, about 4000 IU/mL, about 4500 IU/mL, about 5000 IU/mL, about 5500 IU/mL, about 6000 IU/mL, about 6500 IU/mL, about 7000 IU/mL, about 7500 IU/mL, or about 8000 IU/mL of IL-2. In an embodiment, the cell culture medium comprises between 1000 and 2000 IU/mL, between 2000 and 3000 IU/mL, between 3000 and 4000 IU/mL, between 4000 and 5000 IU/mL, between 5000 and 6000 IU/mL, between 6000 and 7000 IU/mL, between 7000 and 8000 IU/mL, or between 8000 IU/mL of IL-2.

In an embodiment, the cell culture medium comprises OKT3 antibody. In a preferred embodiment, the cell culture medium comprises about 30 ng/mL of OKT3 antibody. In an embodiment, the cell culture medium comprises about 0.1 ng/mL, about 0.5 ng/mL, about 1 ng/mL, about 2.5 ng/mL, about 5 ng/mL, about 7.5 ng/mL, about 10 ng/mL, about 15 ng/mL, about 20 ng/mL, about 25 ng/mL, about 30 ng/mL, about 35 ng/mL, about 40 ng/mL, about 50 ng/mL, about 60 ng/mL, about 70 ng/mL, about 80 ng/mL, about 90 ng/mL, about 100 ng/mL, about 200 ng/mL, about 500 ng/mL, and about 1 μg/mL of OKT3 antibody. In an embodiment, the cell culture medium comprises between 0.1 ng/mL and 1 ng/mL, between 1 ng/mL and 5 ng/mL, between 5 ng/mL and 10 ng/mL, between 10 ng/mL and 20 ng/mL, between 20 ng/mL and 30 ng/mL, between 30 ng/mL and 40 ng/mL, between 40 ng/mL and 50 ng/mL, and between 50 ng/mL and 100 ng/mL of OKT3 antibody.

In some embodiments, IL-2, IL-7, IL-15, and IL-21 as well as combinations thereof can be included during the second expansion in Step D processes as described herein.

In some embodiments, the second expansion can be conducted in a supplemented cell culture medium comprising IL-2, OKT-3, and antigen-presenting feeder cells.

In some embodiments the antigen-presenting feeder cells (APCs) are PBMCs. In an embodiment, the ratio of TILs to PBMCs and/or antigen-presenting cells in the rapid expansion and/or the second expansion is about 1 to 25, about 1 to 50, about 1 to 100, about 1 to 125, about 1 to 150, about 1 to 175, about 1 to 200, about 1 to 225, about 1 to 250, about 1 to 275, about 1 to 300, about 1 to 325, about 1 to 350, about 1 to 375, about 1 to 400, or about 1 to 500. In an embodiment, the ratio of TILs to PBMCs in the rapid expansion and/or the second expansion is between 1 to 50 and 1 to 300. In an embodiment, the ratio of TILs to PBMCs in the rapid expansion and/or the second expansion is between 1 to 100 and 1 to 200.

In an embodiment, REP and/or the second expansion is performed in flasks with the bulk TILs being mixed with a 100- or 200-fold excess of inactivated feeder cells, 30 mg/mL OKT3 anti-CD3 antibody and 3000 IU/mL IL-2 in 150 ml media. Media replacement is done (generally ⅔ media replacement via respiration with fresh media) until the cells are transferred to an alternative growth chamber. Alternative growth chambers include GRex flasks and gas permeable containers as more fully discussed below.

In some embodiments, the second expansion (also referred to as the REP process) is shortened to 7-14 days, as discussed in the examples and figures. In some embodiments, the second expansion is shortened to 11 days.

In an embodiment, REP and/or the second expansion may be performed using T-175 flasks and gas permeable bags as previously described (Tran, et al., J. Immunother. 2008, 31, 742-51; Dudley, et al., J. Immunother. 2003, 26, 332-42) or gas permeable cultureware (G-Rex flasks). For TIL rapid expansion and/or second expansion in T-175 flasks, 1×10⁶ TILs suspended in 150 mL of media may be added to each T-175 flask. The TILs may be cultured in a 1 to 1 mixture of CM and AIM-V medium, supplemented with 3000 IU per mL of IL-2 and 30 ng per ml of anti-CD3. The T-175 flasks may be incubated at 37° C. in 5% CO₂. Half the media may be exchanged on day 5 using 50/50 medium with 3000 IU per mL of IL-2. On day 7 cells from two T-175 flasks may be combined in a 3 L bag and 300 mL of AIM V with 5% human AB serum and 3000 IU per mL of IL-2 was added to the 300 ml of TIL suspension. The number of cells in each bag was counted every day or two and fresh media was added to keep the cell count between 0.5 and 2.0×10⁶ cells/mL.

In an embodiment, REP and/or the second expansion may be performed in 500 mL capacity gas permeable flasks with 100 cm gas-permeable silicon bottoms (G-Rex 100, commercially available from Wilson Wolf Manufacturing Corporation, New Brighton, MN, USA), 5×10⁶ or 10×10⁶ TIL may be cultured with PBMCs in 400 mL of 50/50 medium, supplemented with 5% human AB serum, 3000 IU per mL of IL-2 and 30 ng per ml of anti-CD3 (OKT3). The G-Rex 100 flasks may be incubated at 37° C. in 5% CO₂. On day 5, 250 mL of supernatant may be removed and placed into centrifuge bottles and centrifuged at 1500 rpm (491×g) for 10 minutes. The TIL pellets may be re-suspended with 150 mL of fresh medium with 5% human AB serum, 3000 IU per mL of IL-2, and added back to the original G-Rex 100 flasks. When TIL are expanded serially in G-Rex 100 flasks, on day 7 the TIL in each G-Rex 100 may be suspended in the 300 mL of media present in each flask and the cell suspension may be divided into 3 100 mL aliquots that may be used to seed 3 G-Rex 100 flasks. Then 150 mL of AIM-V with 5% human AB serum and 3000 IU per mL of IL-2 may be added to each flask. The G-Rex 100 flasks may be incubated at 37° C. in 5% CO₂ and after 4 days 150 mL of AIM-V with 3000 IU per mL of IL-2 may be added to each G-Rexl OO flask. The cells may be harvested on day 14 of culture.

In an embodiment, REP and/or the second expansion is performed in flasks with the bulk TILs being mixed with a 100- or 200-fold excess of inactivated feeder cells, 30 mg/mL OKT3 anti-CD3 antibody and 3000 IU/mL IL-2 in 150 ml media. Media replacement is done (generally ⅔ media replacement via respiration with fresh media) until the cells are transferred to an alternative growth chamber. Alternative growth chambers include GRex flasks and gas permeable containers as more fully discussed below.

In an embodiment, REP and/or the second expansion is performed and further comprises a step wherein TILs are selected for superior tumor reactivity. Any selection method known in the art may be used. For example, the methods described in U.S. Patent Application Publication No. 2016/0010058 A1, the disclosures of which are incorporated herein by reference, may be used for selection of TILs for superior tumor reactivity.

REP and/or the second expansion of TIL can be performed using T-175 flasks and gas-permeable bags as previously described (Tran K Q, Zhou J, Durflinger K H, et al., 2008, J Immunother., 31:742-751, and Dudley M E, Wunderlich J R, Shelton T E, et al. 2003, J Immunother., 26:332-342) or gas-permeable G-Rex flasks. In some embodiments, REP and/or the second expansion is performed using flasks. In some embodiments, REP is performed using gas-permeable G-Rex flasks. For TIL REP and/or the second expansion in T-175 flasks, about 1×10⁶ TIL are suspended in about 150 mL of media and this is added to each T-175 flask. The TIL are cultured with irradiated (50 Gy) allogeneic PBMC as “feeder” cells at a ratio of 1 to 100 and the cells were cultured in a 1 to 1 mixture of CM and AIM-V medium (50/50 medium), supplemented with 3000 IU/mL of IL-2 and 30 ng/mL of anti-CD3. The T-175 flasks are incubated at 37° C. in 5% CO₂. In some embodiments, half the media is changed on day 5 using 50/50 medium with 3000 IU/mL of IL-2. In some embodiments, on day 7, cells from 2 T-175 flasks are combined in a 3 L bag and 300 mL of AIM-V with 5% human AB serum and 3000 IU/mL of IL-2 is added to the 300 mL of TIL suspension. The number of cells in each bag can be counted every day or two and fresh media can be added to keep the cell count between about 0.5 and about 2.0×10⁶ cells/mL.

For TIL REP and/or the second expansion in 500 mL capacity flasks with 100 cm² gas-permeable silicon bottoms (G-Rex100, Wilson Wolf) (FIG. 1), about 5×10⁶ or 10×10⁶ TIL are cultured with irradiated allogeneic PBMC at a ratio of 1 to 100 in 400 mL of 50/50 medium, supplemented with 3000 IU/mL of IL-2 and 30 ng/mL of anti-CD3. The G-Rex100 flasks are incubated at 37° C. in 5% CO₂. In some embodiments, on day 5, 250 mL of supernatant is removed and placed into centrifuge bottles and centrifuged at 1500 rpm (491 g) for 10 minutes. The TIL pellets can then be resuspended with 150 mL of fresh 50/50 medium with 3000 IU/mL of IL-2 and added back to the original G-Rex100 flasks. In embodiments where TILs are expanded serially in G-Rex100 flasks, on day 7 the TIL in each G-Rex100 are suspended in the 300 mL of media present in each flask and the cell suspension was divided into three 100 mL aliquots that are used to seed 3 G-Rex100 flasks. Then 150 mL of AIM-V with 5% human AB serum and 3000 IU/mL of IL-2 is added to each flask. The G-Rex100 flasks are incubated at 37° C. in 5% CO₂ and after 4 days 150 mL of AIM-V with 3000 IU/mL of IL-2 is added to each G-Rex100 flask. The cells are harvested on day 14 of culture.

In an embodiment, the second expansion procedures described herein (Step D, including REP) require an excess of feeder cells during REP TIL expansion and/or during the second expansion. In many embodiments, the feeder cells are peripheral blood mononuclear cells (PBMCs) obtained from standard whole blood units from healthy blood donors. The PBMCs are obtained using standard methods such as Ficoll-Paque gradient separation.

In general, the allogenic PBMCs are inactivated, either via irradiation or heat treatment, and used in the REP procedures, as described in the examples, in particular example 14, which provides an exemplary protocol for evaluating the replication incompetence of irradiate allogeneic PBMCs.

In some embodiments, PBMCs are considered replication incompetent and accepted for use in the TIL expansion procedures described herein if the total number of viable cells on day 14 is less than the initial viable cell number put into culture on day 0 of the REP and/or day 0 of the second expansion (i.e., the start day of the second expansion).

In some embodiments, PBMCs are considered replication incompetent and accepted for use in the TIL expansion procedures described herein if the total number of viable cells, cultured in the presence of OKT3 and IL-2, on day 7 and day 14 has not increased from the initial viable cell number put into culture on day 0 of the REP and/or day 0 of the second expansion (i.e., the start day of the second expansion). In some embodiments, the PBMCs are cultured in the presence of 30 ng/ml OKT3 antibody and 3000 IU/ml IL-2.

In some embodiments, PBMCs are considered replication incompetent and accepted for use in the TIL expansion procedures described herein if the total number of viable cells, cultured in the presence of OKT3 and IL-2, on day 7 and day 14 has not increased from the initial viable cell number put into culture on day 0 of the REP and/or day 0 of the second expansion (i.e., the start day of the second expansion). In some embodiments, the PBMCs are cultured in the presence of 5-60 ng/ml OKT3 antibody and 1000-6000 IU/ml IL-2. In some embodiments, the PBMCs are cultured in the presence of 10-50 ng/ml OKT3 antibody and 2000-5000 IU/ml IL-2. In some embodiments, the PBMCs are cultured in the presence of 20-40 ng/ml OKT3 antibody and 2000-4000 IU/ml IL-2. In some embodiments, the PBMCs are cultured in the presence of 25-35 ng/ml OKT3 antibody and 2500-3500 IU/ml IL-2.

In an embodiment, artificial antigen presenting cells are used in the REP stage as a replacement for, or in combination with, PBMCs.

The expansion methods described herein generally use culture media with high doses of a cytokine, in particular IL-2, as is known in the art.

Alternatively, using combinations of cytokines for the rapid expansion and or second expansion of TILs is additionally possible, with combinations of two or more of IL-2, IL-15 and IL-21 as is generally outlined in International Publication No. WO 2015/189356 and W International Publication No. WO 2015/189357, hereby expressly incorporated by reference in their entirety. Thus, possible combinations include IL-2 and IL-15, IL-2 and IL-21, IL-15 and IL-21 and IL-2, IL-15 and IL-21, with the latter finding particular use in many embodiments. The use of combinations of cytokines specifically favors the generation of lymphocytes, and in particular T-cells as described therein.

In some embodiments, the culture media used in expansion methods described herein (including REP) also includes an anti-CD3 antibody. An anti-CD3 antibody in combination with IL-2 induces T cell activation and cell division in the TIL population. This effect can be seen with full length antibodies as well as Fab and F(ab′)2 fragments, with the former being generally preferred; see, e.g., Tsoukas et al., J. Immunol. 1985, 135, 1719, hereby incorporated by reference in its entirety.

As will be appreciated by those in the art, there are a number of suitable anti-human CD3 antibodies that find use in the invention, including anti-human CD3 polyclonal and monoclonal antibodies from various mammals, including, but not limited to, murine, human, primate, rat, and canine antibodies. In particular embodiments, the OKT3 anti-CD3 antibody is used (commercially available from Ortho-McNeil, Raritan, NJ or Miltenyi Biotech, Auburn, CA).

After the second expansion step, cells can be harvested. In some embodiments the TILs are harvested after one, two, three, four or more second expansion steps.

TILs can be harvested in any appropriate and sterile manner, including for example by centrifugation. Methods for TIL harvesting are well known in the art and any such know methods can be employed with the present process. In some embodiments, TILs are harvested using an automated system. In some embodiments, TILs are harvest using a semi-automated system. In some embodiments, the TILs from the second expansion are harvested using a semi-automated machine. In some embodiments, the LOVO system is employed (commercially available from Benchmark Electronics, for example). In some embodiments, the harvesting step includes wash the TILs, formulating the TILs, and/or aliquoting the TILs. In some embodiments, the cells are optionally frozen after harvesting or as part of harvesting.

After Steps A through E are complete, cells are transferred to a container for use in administration to a patient.

In an embodiment, TILs expanded using APCs of the present disclosure are administered to a patient as a pharmaceutical composition. In an embodiment, the pharmaceutical composition is a suspension of TILs in a sterile buffer. TILs expanded using PBMCs of the present disclosure may be administered by any suitable route as known in the art. In some embodiments, the T-cells are administered as a single intra-arterial or intravenous infusion, which preferably lasts approximately 30 to 60 minutes. Other suitable routes of administration include intraperitoneal, intrathecal, and intralymphatic.

As will be appreciated, any of the steps A through F described above can be repeated any number of times and may in addition be conducted in different orders than described above.

In some embodiments, one or more of the expansion steps may be repeated prior to the Final Formulation Step F. Such additional expansion steps may include the elements of the first and/or second expansion steps described above (e.g., include the described components in the cell culture medium). The additional expansion steps may further include additional elements, including additional components in the cell culture medium that are supplemented into the cell culture medium before and/or during the additional expansion steps.

In further embodiments, any of the expansion steps described in FIG. 14 and in the above paragraphs may be preceded or followed by a cryopreservation step in which the cells produced during an expansion step are preserved using methods known in the art for storage until needed for the remaining steps of the manufacturing/expansion process.

In an embodiment, the invention includes a kit for expanding TILs according to any of the foregoing methods.

### Pharmaceutical Compositions, Dosages, and Dosing Regimens for TILs

In an embodiment, TILs expanded using processes and methods of the present disclosure are administered to a patient as a pharmaceutical composition. In an embodiment, the pharmaceutical composition is a suspension of TILs in a sterile buffer. TILs expanded using processes and methods of the present disclosure may be administered by any suitable route as known in the art. Preferably, the TILs are administered as a single intra-arterial or intravenous infusion, which preferably lasts approximately 30 to 60 minutes. Other suitable routes of administration include intraperitoneal, intrathecal, and intralymphatic administration.

Any suitable dose of TILs can be administered. Preferably, from about 2.3×10¹⁰ to about 13.7×10¹⁰ TILs are administered, with an average of around 7.8×10¹⁰ TILs, particularly if the cancer is melanoma. In an embodiment, about 1.2×10¹⁰ to about 4.3×10¹⁰ of TILs are administered.

In some embodiments, the number of the TILs provided in the pharmaceutical compositions of the invention is about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰4×10¹⁰5×10¹⁰6×10¹⁰, 7×10¹⁰8×10¹⁰, 9×10¹⁰, 1×10¹, 2×10¹¹, 3×10¹¹, 4×10¹¹, 5×10¹¹, 6×10¹¹, 7×10¹¹, 8×10¹¹, 9×10¹¹1×10¹², 2×10¹², 3×10¹², 4×10¹²5×10¹², 6×10¹², 7×10¹²8×10¹², 9×10¹², 10×10¹³, 2×10¹³, 3×10¹³4×10¹³, 5×10¹³6×10¹³, 7×10¹³, 8×10¹³, and 9×10¹³. In an embodiment, the number of the TILs provided in the pharmaceutical compositions of the invention is in the range of 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, 1×10¹⁰ to 5×10¹⁰, 5×10¹⁰ to 1×10¹¹, 5×10¹¹ to 1×10¹², 1×10¹² to 5×10¹², and 5×10¹² to 1×10¹³.

In some embodiments, the concentration of the TILs provided in the pharmaceutical compositions of the invention is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of the TILs provided in the pharmaceutical compositions of the invention is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of the TILs provided in the pharmaceutical compositions of the invention is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of the TILs provided in the pharmaceutical compositions of the invention is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of the TILs provided in the pharmaceutical compositions of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the TILs provided in the pharmaceutical compositions of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

The TILs provided in the pharmaceutical compositions of the invention are effective over a wide dosage range. The exact dosage will depend upon the route of administration, the form in which the compound is administered, the gender and age of the subject to be treated, the body weight of the subject to be treated, and the preference and experience of the attending physician. The clinically-established dosages of the TILs may also be used if appropriate. The amounts of the pharmaceutical compositions administered using the methods herein, such as the dosages of TILs, will be dependent on the human or mammal being treated, the severity of the disorder or condition, the rate of administration, the disposition of the active pharmaceutical ingredients and the discretion of the prescribing physician.

In some embodiments, TILs may be administered in a single dose. Such administration may be by injection, e.g., intravenous injection. In some embodiments, TILs may be administered in multiple doses. Dosing may be once, twice, three times, four times, five times, six times, or more than six times per year. Dosing may be once a month, once every two weeks, once a week, or once every other day. Administration of TILs may continue as long as necessary.

In some embodiments, an effective dosage of TILs is about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹°, 3×10¹⁰4×10¹⁰5×10¹° 6×10¹⁰, 7×10¹⁰, 8×10¹⁰, 9×10¹⁰, ×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹²4×10¹², 5×10¹²6×10¹², 7×10¹²8×10¹², 9×10¹²1×10¹³, 2×10¹³, 3×10¹³, 4×10¹³, 5×10¹³6×10¹³, 7×10¹³, 8×10¹³, and 9×10¹³. In some embodiments, an effective dosage of TILs is in the range of 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, 1×10¹⁰ to 5×10¹⁰, 5×10¹⁰ to 1×10¹¹, 5×10¹¹ to 1×10¹², 1×10¹² to 5×10¹², and 5×10¹² to 1×10¹.

In some embodiments, an effective dosage of TILs is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg.

In some embodiments, an effective dosage of TILs is in the range of about 1 mg to about 500 mg, about 10 mg to about 300 mg, about 20 mg to about 250 mg, about 25 mg to about 200 mg, about 1 mg to about 50 mg, about 5 mg to about 45 mg, about 10 mg to about 40 mg, about 15 mg to about 35 mg, about 20 mg to about 30 mg, about 23 mg to about 28 mg, about 50 mg to about 150 mg, about 60 mg to about 140 mg, about 70 mg to about 130 mg, about 80 mg to about 120 mg, about 90 mg to about 110 mg, or about 95 mg to about 105 mg, about 98 mg to about 102 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 207 mg.

An effective amount of the TILs may be administered in either single or multiple doses by any of the accepted modes of administration of agents having similar utilities, including intranasal and transdermal routes, by intra-arterial injection, intravenously, intraperitoneally, parenterally, intramuscularly, subcutaneously, topically, by transplantation or direct injection into tumor, or by inhalation.

### Pharmaceutical Compositions, Dosages, and Dosing Regimens for TNFRSF Agonists

In one embodiment, the invention provides a pharmaceutical composition for use in the treatment of the diseases and conditions described herein. In a preferred embodiment, the invention provides pharmaceutical compositions, including those described below, for use in the treatment of a hyperproliferative disease. In a preferred embodiment, the invention provides pharmaceutical compositions, including those described below, for use in the treatment of cancer.

In some embodiments, a TNFRSF agonist antibody formulation comprises one or more excipients selected from tris-hydrochloride, sodium chloride, mannitol, pentetic acid, polysorbate 80, sodium hydroxide, and hydrochloric acid.

In an embodiment, a TNFRSF agonist is administered to a subject by infusing a dose selected from the group consisting of about 5 mg, about 8 mg, about 10 mg, about 20 mg, about 25 mg, about 50 mg, about 75 mg, 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, and about 2000 mg. In an embodiment, a TNFRSF agonist is administered weekly. In an embodiment, a TNFRSF agonist is administered every two weeks. In an embodiment, a TNFRSF agonist is administered every three weeks. In an embodiment, a TNFRSF agonist is administered monthly. In an embodiment, a TNFRSF agonist is administered intravenously in a dose of 8 mg given every three weeks for 4 doses over a 12-week period. In an embodiment, a TNFRSF agonist is administered at a lower initial dose, which is escalated when administered at subsequent intervals administered monthly. For example, the first infusion can deliver 300 mg of a TNFRSF agonist, and subsequent weekly doses could deliver 2,000 mg of a TNFRSF agonist for eight weeks, followed by monthly doses of 2,000 mg of a TNFRSF agonist.

The amounts of TNFRSF agonists administered will be dependent on the human or mammal being treated, the severity of the disorder or condition, the rate of administration, the disposition of the compounds and the discretion of the prescribing physician. However, an effective dosage of each is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day. The dosage of the TNFRSF agonist(s) may be provided in units of mg/kg of body mass or in mg/m² of body surface area. In an embodiment, a TNFRSF agonist and a second TNFRSF agonist are delivered in mg/kg or in mg/m² in a ration selected from the group consisting of about 20:1, about 19:1, about 18:1, about 17:1, about 16:1, about 15:1, about 14:1, about 13:1, about 12:1, about 11:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, and about 1:20.

In some embodiments, the combination of TILs and a TNFRSF agonist is administered in a single dose. Such administration may be by injection, e.g., intravenous injection, in order to introduce the TNFRSF agonist.

In some embodiments, the combination of TILs and TNFRSF agonists is administered in multiple doses. In a preferred embodiment, the combination of TILs and TNFRSF agonists is administered in multiple doses. Dosing of the TNFRSF agonists may be once, twice, three times, four times, five times, six times, or more than six times per day. Dosing of TILs and TNFRSF agonists may be once a month, once every two weeks, once a week, or once every other day.

In selected embodiments, the TNFRSF agonists are administered for more than 1, 2, 3, 4, 5, 6, 7, 14, 28 days, 2 months, 3 months, 6 months, 12 months, or 24 months. In some cases, continuous dosing is achieved and maintained as long as necessary.

In some embodiments, an effective dosage of a TNFRSF agonist disclosed herein is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg. In some embodiments, an effective dosage of a TNFRSF agonist disclosed herein is about 25 mg, about 50 mg, about 75 mg, about 100 mg, about 125 mg, about 150 mg, about 175 mg, about 200 mg, about 225 mg, or about 250 mg.

In some embodiments, an effective dosage of a TNFRSF agonist disclosed herein is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg. In some embodiments, an effective dosage of a TNFRSF agonist disclosed herein is about 0.35 mg/kg, about 0.7 mg/kg, about 1 mg/kg, about 1.4 mg/kg, about 1.8 mg/kg, about 2.1 mg/kg, about 2.5 mg/kg, about 2.85 mg/kg, about 3.2 mg/kg, or about 3.6 mg/kg.

In some embodiments, an effective dosage of a TNFRSF agonist disclosed herein is in the range of about 1 mg to about 500 mg, about 10 mg to about 300 mg, about 20 mg to about 250 mg, about 25 mg to about 200 mg, about 1 mg to about 50 mg, about 5 mg to about 45 mg, about 10 mg to about 40 mg, about 15 mg to about 35 mg, about 20 mg to about 30 mg, about 23 mg to about 28 mg, about 50 mg to about 150 mg, about 60 mg to about 140 mg, about 70 mg to about 130 mg, about 80 mg to about 120 mg, about 90 mg to about 110 mg, or about 95 mg to about 105 mg, about 98 mg to about 102 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 207 mg. In some embodiments, an effective dosage of a TNFRSF agonist disclosed herein is about 25 mg, about 50 mg, about 75 mg, about 100 mg, about 125 mg, about 150 mg, about 175 mg, about 200 mg, about 225 mg, or about 250 mg.

In some embodiments, an effective dosage of a TNFRSF agonist disclosed herein is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.01 mg/kg to about 0.7 mg/kg, about 0.07 mg/kg to about 0.65 mg/kg, about 0.15 mg/kg to about 0.6 mg/kg, about 0.2 mg/kg to about 0.5 mg/kg, about 0.3 mg/kg to about 0.45 mg/kg, about 0.3 mg/kg to about 0.4 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg to about 1.6 mg/kg, about 1.35 mg/kg to about 1.5 mg/kg, about 1.4 mg/kg to about 1.45 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg. In some embodiments, a TNFRSF agonist disclosed herein is about 0.4 mg/kg, about 0.7 mg/kg, about 1 mg/kg, about 1.4 mg/kg, about 1.8 mg/kg, about 2.1 mg/kg, about 2.5 mg/kg, about 2.85 mg/kg, about 3.2 mg/kg, or about 3.6 mg/kg.

In some embodiments, a TNFRSF agonist is administered at a dosage of 10 to 1000 mg BID, including 10, 20, 30, 40, 50, 60, 70, 80, 90, 100, 150, or 200 mg BID.

In some embodiments, the concentration of the TNFRSF agonists, and combinations thereof provided in the pharmaceutical compositions of the invention is independently less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of the TNFRSF agonists, and combinations thereof provided in the pharmaceutical compositions of the invention is independently greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of the TNFRSF agonists in pharmaceutical compositions is independently in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of the TNFRSF agonists in pharmaceutical compositions is independently in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the concentration of the TNFRSF agonists in pharmaceutical compositions is independently equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the concentration of the TNFRSF agonists in pharmaceutical compositions is independently more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5 g, 4 g, 4.5 g, 5 g, 5.5 g, 6 g, 6.5 g, 7 g, 7.5 g, 8 g, 8.5 g, 9 g, 9.5 g, or 10 g.

Described below are non-limiting pharmaceutical compositions and methods for preparing the same.

### Pharmaceutical Compositions for Injection

In preferred embodiments, the invention provides a pharmaceutical composition for injection containing the combination of a TIL and at least one TNFRSF agonist, and combinations thereof, and a pharmaceutical excipient suitable for injection, including intratumoral injection or intravenous infusion. Components and amounts of agents in the compositions are as described herein.

The forms in which the compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid and thimerosal.

Sterile injectable solutions are prepared by incorporating the combination of the TNFRSF agonists and TILs in the required amounts in the appropriate media with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Other Pharmaceutical Compositions

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; and Pratt and Taylor, eds., Principles of Drug Action, Third Edition, Churchill Livingston, N.Y., 1990, each of which is incorporated by reference herein in its entirety.

Administration of a combination of a TIL and a TNFRSF agonist can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. The combination of compounds can also be administered intraadiposally or intrathecally.

The invention also provides kits. The kits include a combination of ready-to-administer TILs and a TNFRSF agonist, either alone or in combination in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. In selected embodiments, the TNFRSF agonists and TILs and another active pharmaceutical ingredient are provided as separate compositions in separate containers within the kit. In selected embodiments, the molecule selected from the group consisting of a TNFRSF agonist and the TILs are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in selected embodiments, be marketed directly to the consumer.

The kits described above are preferably for use in the treatment of the diseases and conditions described herein. In a preferred embodiment, the kits are for use in the treatment of cancer. In preferred embodiments, the kits are for use in treating solid tumor cancers, lymphomas and leukemias.

In a preferred embodiment, the kits of the present invention are for use in the treatment of cancer, including any of the cancers described herein.

### Methods of Treating Cancers

The compositions and combinations of TILs and TNFRSF agonists described herein can be used in a method for treating hyperproliferative disorders. In a preferred embodiment, they are for use in treating cancers. In a preferred embodiment, the invention provides a method of treating a cancer and compositions and combinations of TILs and TNFRSF agonists for treating a cancer, wherein the cancer is selected from the group consisting of melanoma, ovarian cancer, cervical cancer, lung cancer, bladder cancer, breast cancer, head and neck cancer, renal cell carcinoma, acute myeloid leukemia, colorectal cancer, and sarcoma. In a preferred embodiment, the invention provides a method of treating a cancer and compositions and combinations of TILs and TNFRSF agonists for treating a cancer, wherein the cancer is selected from the group consisting of non-small cell lung cancer (NSCLC) or triple negative breast cancer, double-refractory melanoma, and uveal (ocular) melanoma. In a preferred embodiment, the invention provides a method of treating a cancer wherein the cancer is selected from the group consisting of melanoma, ovarian cancer, cervical cancer, lung cancer, bladder cancer, breast cancer, head and neck cancer (head and neck squamous cell cancer), renal cell carcinoma, acute myeloid leukemia, colorectal cancer, cholangiocarcinoma, and sarcoma with a combination of TILs and a TNFRSF agonist. In a preferred embodiment, the invention provides compositions and combinations of TILs and TNFRSF agonists for treating a cancer wherein the cancer is selected from the group consisting of melanoma, ovarian cancer, cervical cancer, lung cancer, bladder cancer, breast cancer, head and neck cancer, renal cell carcinoma, acute myeloid leukemia, colorectal cancer, cholangiocarcinoma, and sarcoma. In a preferred embodiment, the invention provides a method of treating a cancer, wherein the cancer is selected from the group consisting of non-small cell lung cancer (NSCLC) or triple negative breast cancer, double-refractory melanoma, and uveal (ocular) melanoma with a combination of TILs and a TNFRSF agonist. In a preferred embodiment, the invention provides compositions and combinations of TILs and TNFRSF agonists for treating a cancer wherein the cancer is selected from the group consisting of non-small cell lung cancer (NSCLC) or triple negative breast cancer, double-refractory melanoma, and uveal (ocular) melanoma. In an embodiment, the TILs are expanded by a process described herein.

In some embodiments, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs; and
(f) administering a therapeutically effective portion of the third population of TILs to a patient with the cancer;
wherein the cancer is selected from the group consisting of melanoma, ovarian cancer, cervical cancer, lung cancer, bladder cancer, breast cancer, head and neck cancer, renal cell carcinoma, acute myeloid leukemia, colorectal cancer, cholangiocarcinoma, sarcoma, non-small cell lung cancer (NSCLC) or triple negative breast cancer, double-refractory melanoma, and uveal (ocular) melanoma.

In some embodiments, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and wherein the rapid expansion is performed over a period of 14 days or less;
(e) harvesting the third population of TILs; and
(f) administering a therapeutically effective portion of the third population of TILs to a patient with the cancer;
wherein the cancer is selected from the group consisting of melanoma, ovarian cancer, cervical cancer, lung cancer, bladder cancer, breast cancer, head and neck cancer, renal cell carcinoma, acute myeloid leukemia, colorectal cancer, cholangiocarcinoma, sarcoma, non-small cell lung cancer (NSCLC) or triple negative breast cancer, double-refractory melanoma, and uveal (ocular) melanoma.

In some embodiments, the invention provides a method of treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and wherein the rapid expansion is performed over a period of 14 days or less;
(e) harvesting the third population of TILs; and
(f) administering a therapeutically effective portion of the third population of TILs to a patient with the cancer;
wherein the cancer is uveal (ocular) melanoma.

In an embodiment, the invention includes a kit for treating a cancer with a population of TILs according to any of the foregoing methods.

Efficacy of the methods, compounds, and combinations of compounds described herein in treating, preventing and/or managing the indicated diseases or disorders can be tested using various animal models known in the art. Models for determining efficacy of treatments for pancreatic cancer are described in Herreros-Villanueva, et al., World J. Gastroenterol. 2012, 18, 1286-1294. Models for determining efficacy of treatments for breast cancer are described, e.g., in Fantozzi, Breast Cancer Res. 2006, 8, 212. Models for determining efficacy of treatments for ovarian cancer are described, e.g., in Mullany, et al., Endocrinology 2012, 153, 1585-92; and Fong, et al., J. Ovarian Res. 2009, 2, 12. Models for determining efficacy of treatments for melanoma are described, e.g., in Damsky, et al., Pigment Cell & Melanoma Res. 2010, 23, 853-859. Models for determining efficacy of treatments for lung cancer are described, e.g., in Meuwissen, et al., Genes & Development, 2005, 19, 643-664. Models for determining efficacy of treatments for lung cancer are described, e.g., in Kim, Clin. Exp. Otorhinolaryngol. 2009, 2, 55-60; and Sano, Head Neck Oncol. 2009, 1, 32. Models for determining efficacy of treatments for colorectal cancer, including the CT26 model, are described in Castle, et al., BMC Genomics, 2013, 15, 190; Endo, et al., Cancer Gene Therapy, 2002, 9, 142-148; Roth, et al., Adv. Immunol. 1994, 57, 281-351; Fearon, et al., Cancer Res. 1988, 48, 2975-2980.

### Co-Administration of IL-2

In an embodiment, the invention provides a method treating a cancer with a population of tumor infiltrating lymphocytes (TILs) comprising the steps of:
(a) resecting a tumor from a patient;
(b) obtaining a first population of TILs from the tumor;
(c) performing an initial expansion of the first population of TILs in a first cell culture medium to obtain a second population of TILs, wherein the second population of TILs is at least 5-fold greater in number than the first population of TILs, wherein the first cell culture medium comprises IL-2 and a tumor necrosis factor receptor superfamily (TNFRSF) agonist, and wherein the initial expansion is performed over a period of 21 days or less;
(d) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), peripheral blood mononuclear cells (PBMCs), and optionally the TNFRSF agonist, and wherein the rapid expansion is performed over a period of 14 days or less;
(e) harvesting the third population of TILs;
(f) administering a therapeutically effective portion of the third population of TILs to the patient with the cancer; and
(g) administering an IL-2 regimen to the patient.

In an embodiment, the IL-2 regimen comprises a high-dose IL-2 regimen, wherein the high-dose IL-2 regimen comprises aldesleukin, or a biosimilar or variant thereof, administered intravenously starting on the day after administering a therapeutically effective portion of the third population of TILs, wherein the aldesleukin or a biosimilar or variant thereof is administered at a dose of 600,000 or 720,000 IU/kg (patient body mass) using 15-minute bolus intravenous infusions every eight hours until tolerance, for a maximum of 14 doses. Following 9 days of rest, this schedule may be repeated for another 14 doses, for a maximum of 28 doses in total.

In an embodiment, the IL-2 regimen comprises a high-dose IL-2 regimen, wherein the high-dose IL-2 regimen comprises aldesleukin, or a biosimilar or variant thereof, administered intravenously starting on the day after administering a therapeutically effective portion of the third population of TILs, wherein the aldesleukin or a biosimilar or variant thereof is administered at a dose of 0.037 mg/kg or 0.044 mg/kg IU/kg (patient body mass) using 15-minute bolus intravenous infusions every eight hours until tolerance, for a maximum of 14 doses. Following 9 days of rest, this schedule may be repeated for another 14 doses, for a maximum of 28 doses in total.

In an embodiment, the IL-2 regimen comprises a decrescendo IL-2 regimen. Decrescendo IL-2 regimens have been described in O'Day, et al., J. Clin. Oncol. 1999, 17, 2752-61 and Eton, et al., Cancer 2000, 88, 1703-9, the disclosures of which are incorporated herein by reference. In an embodiment, a decrescendo IL-2 regimen comprises 18×10⁶ IU/m² administered intravenously over 6 hours, followed by 18×10⁶ IU/m² administered intravenously over 12 hours, followed by 18×10⁶ IU/m² administered intravenously over 24 hrs, followed by 4.5×10⁶ IU/m² administered intravenously over 72 hours. This treatment cycle may be repeated every 28 days for a maximum of four cycles. In an embodiment, a decrescendo IL-2 regimen comprises 18,000,000 IU/m² on day 1, 9,000,000 IU/m² on day 2, and 4,500,000 IU/m² on days 3 and 4.

In an embodiment, the IL-2 regimen comprises administration of pegylated IL-2 every 1, 2, 4, 6, 7, 14 or 21 days at a dose of 0.10 mg/day to 50 mg/day.

Non-Myeloablative Lymphodepletion with Chemotherapy

In an embodiment, the invention includes a method of treating a cancer with a population of TILs, wherein a patient is pre-treated with non-myeloablative chemotherapy prior to an infusion of TILs and prior to or concurrent with treatment with a TNFRSF agonist according to the present disclosure. In an embodiment, the non-myeloablative chemotherapy is cyclophosphamide 60 mg/kg/d for 2 days (days 27 and 26 prior to TIL infusion) and fludarabine 25 mg/m²/d for 5 days (days 27 to 23 prior to TIL infusion). In an embodiment, after non-myeloablative chemotherapy and TIL infusion (at day 0) according to the present disclosure, the patient receives an intravenous infusion of IL-2 intravenously at 720,000 IU/kg every 8 hours to physiologic tolerance.

Experimental findings indicate that lymphodepletion prior to adoptive transfer of tumor-specific T lymphocytes plays a key role in enhancing treatment efficacy by eliminating regulatory T cells and competing elements of the immune system (“cytokine sinks”). Accordingly, some embodiments of the invention utilize a lymphodepletion step (sometimes also referred to as “immunosuppressive conditioning”) on the patient prior to the introduction of the reREP TILs of the invention.

In general, lymphodepletion is achieved using administration of fludarabine or cyclophosphamide (the active form being referred to as mafosfamide) and combinations thereof. Such methods are described in Gassner, et al., Cancer Immunol. Immunother. 2011, 60, 75-85, Muranski, et al., Nat. Clin. Pract. Oncol., 2006, 3, 668-681, Dudley, et al., J. Clin. Oncol. 2008, 26, 5233-5239, and Dudley, et al., J. Clin. Oncol. 2005, 23, 2346-2357, all of which are incorporated by reference herein in their entireties.

In some embodiments, the fludarabine is administered at a concentration of 0.5 μg/ml-10 μg/ml fludarabine. In some embodiments, the fludarabine is administered at a concentration of 1 μg/ml fludarabine. In some embodiments, the fludarabine treatment is administered for 1 day, 2 days, 3 days, 4 days, 5 days, 6 days, or 7 days or more. In some embodiments, the fludarabine is administered at a dosage of 10 mg/kg/day, 15 mg/kg/day, 20 mg/kg/day, 25 mg/kg/day, 30 mg/kg/day, 35 mg/kg/day, 40 mg/kg/day, or 45 mg/kg/day. In some embodiments, the fludarabine treatment is administered for 2-7 days at 35 mg/kg/day. In some embodiments, the fludarabine treatment is administered for 4-5 days at 35 mg/kg/day. In some embodiments, the fludarabine treatment is administered for 4-5 days at 25 mg/kg/day.

In some embodiments, the mafosfamide, the active form of cyclophosphamide, is obtained at a concentration of 0.5 μg/mL-10 μg/mL by administration of cyclophosphamide. In some embodiments, mafosfamide, the active form of cyclophosphamide, is obtained at a concentration of 1 μg/mL by administration of cyclophosphamide. In some embodiments, the cyclophosphamide treatment is administered for 1 day, 2 days, 3 days, 4 days, 5 days, 6 days, or 7 days or more. In some embodiments, the cyclophosphamide is administered at a dosage of 100 mg/m²/day, 150 mg/m²/day, 175 mg/m²/day, 200 mg/m²/day, 225 mg/m²/day, 250 mg/m²/day, 275 mg/m²/day, or 300 mg/m²/day. In some embodiments, the cyclophosphamide is administered intravenously (i.e., i.v.) In some embodiments, the cyclophosphamide treatment is administered for 2-7 days at 35 mg/kg/day. In some embodiments, the cyclophosphamide treatment is administered for 4-5 days at 250 mg/m²/day i.v. In some embodiments, the cyclophosphamide treatment is administered for 4 days at 250 mg/m²/day i.v.

In some embodiments, lymphodepletion is performed by administering the fludarabine and the cyclophosphamide are together to a patient. In some embodiments, fludarabine is administered at 25 mg/m²/day i.v. and cyclophosphamide is administered at 250 mg/m²/day i.v. over 4 days.

In an embodiment, the lymphodepletion is performed by administration of cyclophosphamide at a dose of 60 mg/m²/day for two days followed by administration of fludarabine at a dose of 25 mg/m²/day for five days.

Combinations with PD-1 and PD-L1 Inhibitors

Programmed death 1 (PD-1) is a 288-amino acid transmembrane immunocheckpoint receptor protein expressed by T cells, B cells, natural killer (NK) T cells, activated monocytes, and dendritic cells. PD-1, which is also known as CD279, belongs to the CD28 family, and in humans is encoded by the Pdcd1 gene on chromosome 2. PD-1 consists of one immunoglobulin (Ig) superfamily domain, a transmembrane region, and an intracellular domain containing an immunoreceptor tyrosine-based inhibitory motif (ITIM) and an immunoreceptor tyrosine-based switch motif (ITSM). PD-1 and its ligands (PD-L1 and PD-L2) are known to play a key role in immune tolerance, as described in Keir, et al., Annu. Rev. Immunol. 2008, 26, 677-704. PD-1 provides inhibitory signals that negatively regulate T cell immune responses. PD-L1 (also known as B7-H1 or CD274) and PD-L2 (also known as B7-DC or CD273) are expressed on tumor cells and stromal cells, which may be encountered by activated T cells expressing PD-1, leading to immunosuppression of the T cells. PD-L1 is a 290 amino acid transmembrane protein encoded by the Cd274 gene on human chromosome 9. Blocking the interaction between PD-1 and its ligands PD-L1 and PD-L2 by use of a PD-1 inhibitor, a PD-L1 inhibitor, and/or a PD-L2 inhibitor can overcome immune resistance, as demonstrated in recent clinical studies, such as that described in Topalian, et al., N. Eng. J. Med. 2012, 366, 2443-54. PD-L1 is expressed on many tumor cell lines, while PD-L2 is expressed is expressed mostly on dendritic cells and a few tumor lines. In addition to T cells (which inducibly express PD-1 after activation), PD-1 is also expressed on B cells, natural killer cells, macrophages, activated monocytes, and dendritic cells.

The methods, compositions, and combinations of TILs and TNFRSF agonists described herein may also be further combined with programmed death-1 (PD-1), programmed death ligand 1 (PD-L1), and/or programmed death ligand 2 (PD-L2) binding antibodies, antagonists, or inhibitors (i.e., blockers). PD-1, PD-L1, and/or PD-L2 inhibitors may be used in cell culture in conjunction with the TNFRSF agonists described herein during the pre-REP or REP stages of TIL expansion. PD-1, PD-L1, and/or PD-L2 inhibitors may also be used in conjunction with TNFRSF agonists prior to surgical resection of tumor, or during or after infusion of TILs. For example, suitable methods of using PD-1/PD-L1 inhibitors in conjunction with agonistic GITR antibodies and compositions comprising PD-1/PD-L1 antagonists and GITR agonists are described in International Patent Application Publication No. WO 2015/026684 A1, the disclosures of which are incorporated by reference herein.

In an embodiment, the PD-1 inhibitor may be any PD-1 inhibitor or PD-1 blocker known in the art. In particular, it is one of the PD-1 inhibitors or blockers described in more detail in the following paragraphs. The terms “inhibitor,” “antagonist,” and “blocker” are used interchangeably herein in reference to PD-1 inhibitors. For avoidance of doubt, references herein to a PD-1 inhibitor that is an antibody may refer to a compound or antigen-binding fragments, variants, conjugates, or biosimilars thereof. For avoidance of doubt, references herein to a PD-1 inhibitor may also refer to a small molecule compound or a pharmaceutically acceptable salt, ester, solvate, hydrate, cocrystal, or prodrug thereof.

In some embodiments, the compositions and methods described herein include a PD-1 inhibitor. In some embodiments, the PD-1 inhibitor is a small molecule. In a preferred embodiment, the PD-1 inhibitor is an antibody (i.e., an anti-PD-1 antibody), a fragment thereof, including Fab fragments, or a single-chain variable fragment (scFv) thereof. In some embodiments the PD-1 inhibitor is a polyclonal antibody. In a preferred embodiment, the PD-1 inhibitor is a monoclonal antibody. In some embodiments, the PD-1 inhibitor competes for binding with PD-1, and/or binds to an epitope on PD-1. In an embodiment, the antibody competes for binding with PD-1, and/or binds to an epitope on PD-1.

In some embodiments, the compositions and methods described include a PD-1 inhibitor that binds human PD-1 with a K_{D} of about 100 pM or lower, binds human PD-1 with a K_{D} of about 90 pM or lower, binds human PD-1 with a K_{D} of about 80 pM or lower, binds human PD-1 with a K_{D} of about 70 pM or lower, binds human PD-1 with a K_{D} of about 60 pM or lower, binds human PD-1 with a K_{D} of about 50 pM or lower, binds human PD-1 with a K_{D} of about 40 pM or lower, binds human PD-1 with a K_{D} of about 30 pM or lower, binds human PD-1 with a K_{D} of about 20 pM or lower, binds human PD-1 with a K_{D} of about 10 pM or lower, or binds human PD-1 with a K_{D} of about 1 pM or lower.

In some embodiments, the compositions and methods described include a PD-1 inhibitor that binds to human PD-1 with a k_{assoc} of about 7.5×10⁵ l/M·s or faster, binds to human PD-1 with a k_{assoc} of about 7.5×10⁵ l/M·s or faster, binds to human PD-1 with a k_{assoc} of about 8×10⁵ l/M·s or faster, binds to human PD-1 with a k_{assoc} of about 8.5×10⁵ l/M·s or faster, binds to human PD-1 with a k_{assoc} of about 9×10⁵ l/M·s or faster, binds to human PD-1 with a k_{assoc} of about 9.5×10⁵ l/M·s or faster, or binds to human PD-1 with a k_{assoc} of about 1×10⁶ l/M·s or faster.

In some embodiments, the compositions and methods described include a PD-1 inhibitor that binds to human PD-1 with a k_{dissoc} of about 2×10⁻⁵ l/s or slower, binds to human PD-1 with a k_{dissoc} of about 2.1×10⁻⁵ l/s or slower, binds to human PD-1 with a k_{dissoc} of about 2.2×10⁻⁵ l/s or slower, binds to human PD-1 with a k_{dissoc} of about 2.3×10⁻⁵ l/s or slower, binds to human PD-1 with a k_{dissoc} of about 2.4×10⁻⁵ l/s or slower, binds to human PD-1 with a k_{dissoc} of about 2.5×10⁻⁵ l/s or slower, binds to human PD-1 with a k_{dissoc} of about 2.6×10⁻⁵ l/s or slower or binds to human PD-1 with a k_{dissoc} of about 2.7×10⁻⁵ l/s or slower, binds to human PD-1 with a k_{dissoc} of about 2.8×10⁻⁵ l/s or slower, binds to human PD-1 with a k_{dissoc} of about 2.9×10⁻⁵ l/s or slower, or binds to human PD-1 with a k_{dissoc} of about 3×10⁻⁵ l/s or slower.

In some embodiments, the compositions and methods described include a PD-1 inhibitor that blocks or inhibits binding of human PD-L1 or human PD-L2 to human PD-1 with an IC₅₀ of about 10 nM or lower, blocks or inhibits binding of human PD-L1 or human PD-L2 to human PD-1 with an IC₅₀ of about 9 nM or lower, blocks or inhibits binding of human PD-L1 or human PD-L2 to human PD-1 with an IC₅₀ of about 8 nM or lower, blocks or inhibits binding of human PD-L1 or human PD-L2 to human PD-1 with an IC₅₀ of about 7 nM or lower, blocks or inhibits binding of human PD-L1 or human PD-L2 to human PD-1 with an IC₅₀ of about 6 nM or lower, blocks or inhibits binding of human PD-L1 or human PD-L2 to human PD-1 with an IC₅₀ of about 5 nM or lower, blocks or inhibits binding of human PD-L1 or human PD-L2 to human PD-1 with an IC₅₀ of about 4 nM or lower, blocks or inhibits binding of human PD-L1 or human PD-L2 to human PD-1 with an IC₅₀ of about 3 nM or lower, blocks or inhibits binding of human PD-L1 or human PD-L2 to human PD-1 with an IC₅₀ of about 2 nM or lower, or blocks or inhibits binding of human PD-L1 or human PD-L2 to human PD-1 with an IC₅₀ of about 1 nM or lower.

In an embodiment, the PD-1 inhibitor is nivolumab (commercially available as OPDIVO from Bristol-Myers Squibb Co.), or biosimilars, antigen-binding fragments, conjugates, or variants thereof. Nivolumab is a fully human IgG4 antibody blocking the PD-1 receptor. In an embodiment, the anti-PD-1 antibody is an immunoglobulin G4 kappa, anti-(human CD274) antibody. Nivolumab is assigned Chemical Abstracts Service (CAS) registry number 946414-94-4 and is also known as 5C4, BMS-936558, MDX-1106, and ONO-4538. The preparation and properties of nivolumab are described in U.S. Pat. No. 8,008,449 and International Patent Publication No. WO 2006/121168, the disclosures of which are incorporated by reference herein. The clinical safety and efficacy of nivolumab in various forms of cancer has been described in Wang, et al., Cancer Immunol Res. 2014, 2, 846-56; Page, et al., Ann. Rev. Med., 2014, 65, 185-202; and Weber, et al., J. Clin. Oncology, 2013, 31, 4311-4318, the disclosures of which are incorporated by reference herein. The amino acid sequences of nivolumab are set forth in Table 48. Nivolumab has intra-heavy chain disulfide linkages at 22-96,140-196, 254-314, 360-418, 22″-96″, 140″-196″, 254″-314″, and 360″-418″; intra-light chain disulfide linkages at 23′-88′, 134′-194′, 23′″-88′″, and 134′″-194′″; inter-heavy-light chain disulfide linkages at 127-214′, 127″-214′″, inter-heavy-heavy chain disulfide linkages at 219-219″ and 222-222″; and N-glycosylation sites (H CH₂ 84.4) at 290, 290″.

In an embodiment, a PD-1 inhibitor comprises a heavy chain given by SEQ ID NO:463 and a light chain given by SEQ ID NO:464. In an embodiment, a PD-1 inhibitor comprises heavy and light chains having the sequences shown in SEQ ID NO:463 and SEQ ID NO:464, respectively, or antigen binding fragments, Fab fragments, single-chain variable fragments (scFv), variants, or conjugates thereof. In an embodiment, a PD-1 inhibitor comprises heavy and light chains that are each at least 99% identical to the sequences shown in SEQ ID NO:463 and SEQ ID NO:464, respectively. In an embodiment, a PD-1 inhibitor comprises heavy and light chains that are each at least 98% identical to the sequences shown in SEQ ID NO:463 and SEQ ID NO:464, respectively. In an embodiment, a PD-1 inhibitor comprises heavy and light chains that are each at least 97% identical to the sequences shown in SEQ ID NO:463 and SEQ ID NO:464, respectively. In an embodiment, a PD-1 inhibitor comprises heavy and light chains that are each at least 96% identical to the sequences shown in SEQ ID NO:463 and SEQ ID NO:464, respectively. In an embodiment, a PD-1 inhibitor comprises heavy and light chains that are each at least 95% identical to the sequences shown in SEQ ID NO:463 and SEQ ID NO:464, respectively.

In an embodiment, the PD-1 inhibitor comprises the heavy and light chain CDRs or variable regions (VRs) of nivolumab. In an embodiment, the PD-1 inhibitor heavy chain variable region (V_{H}) comprises the sequence shown in SEQ ID NO:465, and the PD-1 inhibitor light chain variable region (V_{L}) comprises the sequence shown in SEQ ID NO:466, and conservative amino acid substitutions thereof. In an embodiment, a PD-1 inhibitor comprises V_{H} and V_{L} regions that are each at least 99% identical to the sequences shown in SEQ ID NO:465 and SEQ ID NO:466, respectively. In an embodiment, a PD-1 inhibitor comprises V_{H} and V_{L} regions that are each at least 98% identical to the sequences shown in SEQ ID NO:465 and SEQ ID NO:466, respectively. In an embodiment, a PD-1 inhibitor comprises V_{H} and V_{L} regions that are each at least 97% identical to the sequences shown in SEQ ID NO:465 and SEQ ID NO:466, respectively. In an embodiment, a PD-1 inhibitor comprises V_{H} and V_{L} regions that are each at least 96% identical to the sequences shown in SEQ ID NO:465 and SEQ ID NO:466, respectively. In an embodiment, a PD-1 inhibitor comprises V_{H} and V_{L} regions that are each at least 95% identical to the sequences shown in SEQ ID NO:465 and SEQ ID NO:466, respectively.

In an embodiment, a PD-1 inhibitor comprises heavy chain CDR1, CDR2 and CDR3 domains having the sequences set forth in SEQ ID NO:467, SEQ ID NO:468, and SEQ ID NO:469, respectively, and conservative amino acid substitutions thereof, and light chain CDR1, CDR2 and CDR3 domains having the sequences set forth in SEQ ID NO:470, SEQ ID NO:471, and SEQ ID NO:472, respectively, and conservative amino acid substitutions thereof. In an embodiment, the antibody competes for binding with, and/or binds to the same epitope on PD-1 as any of the aforementioned antibodies.

In an embodiment, the PD-1 inhibitor is an anti-PD-1 biosimilar monoclonal antibody approved by drug regulatory authorities with reference to nivolumab. In an embodiment, the biosimilar comprises an anti-PD-1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is nivolumab. In some embodiments, the one or more post-translational modifications are selected from one or more of: glycosylation, oxidation, deamidation, and truncation. In some embodiments, the biosimilar is an anti-PD-1 antibody authorized or submitted for authorization, wherein the anti-PD-1 antibody is provided in a formulation which differs from the formulations of a reference medicinal product or reference biological product, wherein the reference medicinal product or reference biological product is nivolumab. The anti-PD-1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is nivolumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is nivolumab.

TABLE 48
Amino acid sequences for PD-1 inhibitors related to nivolumab.

| Identifier | Sequence (One-Letter Amino Acid Symbols) | |
|---|---|---|
| SEQ ID NO: 463 | QVQLVESGGG VVQPGRSLRL DCKASGITFS NSGMHWVRQA PGKGLEWVAV IWYDGSKRYY | 60 |
| nivolumab | ADSVKGRFTI SRDNSKNTLF LQMNSLRAED TAVYYCATND DYWGQGTLVT VSSASTKGPS | 120 |
| heavy chain | VFPLAPCSRS TSESTAALGC LVKDYFPEPV TVSWNSGALT SGVHTFPAVL QSSGLYSLSS | 180 |
| | VVTVPSSSLG TKTYTCNVDH KPSNTKVDKR VESKYGPPCP PCPAPEFLGG PSVFLFPPKP | 240 |
| | KDTLMISRTP EVTCVVVDVS QEDPEVQFNW YVDGVEVHNA KTKPREEQFN STYRVVSVLT | 300 |
| | VLHQDWLNGK EYKCKVSNKG LPSSIEKTIS KAKGQPREPQ VYTLPPSQEE MTKNQVSLTC | 360 |
| | LVKGFYPSDI AVEWESNGQP ENNYKTTPPV LDSDGSFFLY SRLTVDKSRW QEGNVFSCSV | 420 |
| | MHEALHNHYT QKSLSLSLGK | 440 |
| SEQ ID NO: 464 | EIVLTQSPAT LSLSPGERAT LSCRASQSVS SYLAWYQQKP GQAPRLLIYD ASNRATGIPA | 60 |
| nivolumab | RFSGSGSGTD FTLTISSLEP EDFAVYYCQQ SSNWPRTFGQ GTKVEIKRTV AAPSVFIFPP | 120 |
| light chain | SDEQLKSGTA SVVCLLNNFY PREAKVQWKV DNALQSGNSQ ESVTEQDSKD STYSLSSTLT | 180 |
| | LSKADYEKHK VYACEVTHQG LSSPVTKSFN RGEC | 214 |
| SEQ ID NO: 465 | QVQLVESGGG VVQPGRSLRL DCKASGITFS NSGMHWVRQA PGKGLEWVAV IWYDGSKRYY | 60 |
| nivolumab | ADSVKGRFTI SRDNSKNTLF LQMNSLRAED TAVYYCATND DYWGQGTLVT VSS | 113 |
| variable heavy | | |
| chain | | |
| SEQ ID NO: 466 | EIVLTQSPAT LSLSPGERAT LSCRASQSVS SYLAWYQQKP GQAPRLLIYD ASNRATGIPA | 60 |
| nivolumab | RFSGSGSGTD FTLTISSLEP EDFAVYYCQQ SSNWPRTFGQ GTKVEIK | 107 |
| variable light | | |
| chain | | |
| SEQ ID NO: 467 | NSGMH | 5 |
| nivolumab | | |
| heavy chain | | |
| CDR1 | | |
| SEQ ID NO: 468 | VIWYDGSKRY YADSVKG | 17 |
| nivolumab | | |
| heavy chain | | |
| CDR2 | | |
| SEQ ID NO: 469 | NDDY | 4 |
| nivolumab | | |
| heavy chain | | |
| CDR3 | | |
| SEQ ID NO: 470 | RASQSVSSYL A | 11 |
| nivolumab | | |
| light chain | | |
| CDR1 | | |
| SEQ ID NO: 471 | DASNRAT | 7 |
| nivolumab | | |
| light chain | | |
| CDR2 | | |
| SEQ ID NO: 472 | QQSSNWPRT | 9 |
| nivolumab | | |
| light chain | | |
| CDR3 | | |

In another embodiment, the PD-1 inhibitor comprises pembrolizumab (commercially available as KEYTRUDA from Merck & Co., Inc., Kenilworth, NJ, USA), or antigen-binding fragments, conjugates, or variants thereof. Pembrolizumab is assigned CAS registry number 1374853-91-4 and is also known as lambrolizumab, MK-3475, and SCH-900475. Pembrolizumab has an immunoglobulin G4, anti-(human protein PDCD1 (programmed cell death 1)) (human-Mus musculus monoclonal heavy chain), disulfide with human-Mus musculus monoclonal light chain, dimer structure. The structure of pembrolizumab may also be described as immunoglobulin G4, anti-(human programmed cell death 1); humanized mouse monoclonal [228-L-proline(H10-S>P)]γ4 heavy chain (134-218′)-disulfide with humanized mouse monoclonal κ light chain dimer (226-226″:229-229″)-bisdisulfide. The properties, uses, and preparation of pembrolizumab are described in International Patent Publication No. WO 2008/156712 A1, U.S. Pat. No. 8,354,509 and U.S. Patent Application Publication Nos. US 2010/0266617 A1, US 2013/0108651 A1, and US 2013/0109843 A2, the disclosures of which are incorporated herein by reference. The clinical safety and efficacy of pembrolizumab in various forms of cancer is described in Fuerst, Oncology Times, 2014, 36, 35-36; Robert, et al., Lancet, 2014, 384, 1109-17; and Thomas, et al., Exp. Opin. Biol. Ther., 2014, 14, 1061-1064. The amino acid sequences of pembrolizumab are set forth in Table 49. Pembrolizumab includes the following disulfide bridges: 22-96, 22″-96″, 23′-92′, 23′″-92′″, 134-218′, 134″-218′″, 138′-198′, 138′″-198′″, 147-203, 147-203″, 226-226″, 229-229″, 261-321, 261″-321″, 367-425, and 367″-425″, and the following glycosylation sites (N): Asn-297 and Asn-297″. Pembrolizumab is an IgG4/kappa isotype with a stabilizing S228P mutation in the Fc region; insertion of this mutation in the IgG4 hinge region prevents the formation of half molecules typically observed for IgG4 antibodies. Pembrolizumab is heterogeneously glycosylated at Asn297 within the Fc domain of each heavy chain, yielding a molecular weight of approximately 149 kDa for the intact antibody. The dominant glycoform of pembrolizumab is the fucosylated agalacto diantennary glycan form (GOF).

In an embodiment, a PD-1 inhibitor comprises a heavy chain given by SEQ ID NO:473 and a light chain given by SEQ ID NO:474. In an embodiment, a PD-1 inhibitor comprises heavy and light chains having the sequences shown in SEQ ID NO:473 and SEQ ID NO:474, respectively, or antigen binding fragments, Fab fragments, single-chain variable fragments (scFv), variants, or conjugates thereof. In an embodiment, a PD-1 inhibitor comprises heavy and light chains that are each at least 99% identical to the sequences shown in SEQ ID NO:473 and SEQ ID NO:474, respectively. In an embodiment, a PD-1 inhibitor comprises heavy and light chains that are each at least 98% identical to the sequences shown in SEQ ID NO:473 and SEQ ID NO:474, respectively. In an embodiment, a PD-1 inhibitor comprises heavy and light chains that are each at least 97% identical to the sequences shown in SEQ ID NO:473 and SEQ ID NO:474, respectively. In an embodiment, a PD-1 inhibitor comprises heavy and light chains that are each at least 96% identical to the sequences shown in SEQ ID NO:473 and SEQ ID NO:474, respectively. In an embodiment, a PD-1 inhibitor comprises heavy and light chains that are each at least 95% identical to the sequences shown in SEQ ID NO:473 and SEQ ID NO:474, respectively.

In an embodiment, the PD-1 inhibitor comprises the heavy and light chain CDRs or variable regions (VRs) of pembrolizumab. In an embodiment, the PD-1 inhibitor heavy chain variable region (V_{H}) comprises the sequence shown in SEQ ID NO:475, and the PD-1 inhibitor light chain variable region (V_{L}) comprises the sequence shown in SEQ ID NO:476, and conservative amino acid substitutions thereof. In an embodiment, a PD-1 inhibitor comprises V_{H} and V_{L} regions that are each at least 99% identical to the sequences shown in SEQ ID NO:475 and SEQ ID NO:476, respectively. In an embodiment, a PD-1 inhibitor comprises V_{H} and V_{L} regions that are each at least 98% identical to the sequences shown in SEQ ID NO:475 and SEQ ID NO:476, respectively. In an embodiment, a PD-1 inhibitor comprises V_{H} and V_{L} regions that are each at least 97% identical to the sequences shown in SEQ ID NO:475 and SEQ ID NO:476, respectively. In an embodiment, a PD-1 inhibitor comprises V_{H} and V_{L} regions that are each at least 96% identical to the sequences shown in SEQ ID NO:475 and SEQ ID NO:476, respectively. In an embodiment, a PD-1 inhibitor comprises V_{H} and V_{L} regions that are each at least 95% identical to the sequences shown in SEQ ID NO:475 and SEQ ID NO:476, respectively.

In an embodiment, a PD-1 inhibitor comprises heavy chain CDR1, CDR2 and CDR3 domains having the sequences set forth in SEQ ID NO:477, SEQ ID NO:478, and SEQ ID NO:479, respectively, and conservative amino acid substitutions thereof, and light chain CDR1, CDR2 and CDR3 domains having the sequences set forth in SEQ ID NO:480, SEQ ID NO:481, and SEQ ID NO:482, respectively, and conservative amino acid substitutions thereof. In an embodiment, the antibody competes for binding with, and/or binds to the same epitope on PD-1 as any of the aforementioned antibodies.

In an embodiment, the PD-1 inhibitor is an anti-PD-1 biosimilar monoclonal antibody approved by drug regulatory authorities with reference to pembrolizumab. In an embodiment, the biosimilar comprises an anti-PD-1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is pembrolizumab. In some embodiments, the one or more post-translational modifications are selected from one or more of: glycosylation, oxidation, deamidation, and truncation. In some embodiments, the biosimilar is an anti-PD-1 antibody authorized or submitted for authorization, wherein the anti-PD-1 antibody is provided in a formulation which differs from the formulations of a reference medicinal product or reference biological product, wherein the reference medicinal product or reference biological product is pembrolizumab. The anti-PD-1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is pembrolizumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is pembrolizumab.

TABLE 49
Amino acid sequences for PD-1 inhibitors related to pembrolizumab.

| Identifier | Sequence (One-Letter Amino Acid Symbols) | |
|---|---|---|
| SEQ ID NO: 473 | QVQLVQSGVE VKKPGASVKV SCKASGYTFT NYYMYWVRQA PGQGLEWMGG INPSNGGTNF | 60 |
| pembrolizumab | NEKFKNRVTL TTDSSTTTAY MELKSLQFDD TAVYYCARRD YRFDMGFDYW GQGTTVTVSS | 120 |
| heavy chain | ASTKGPSVFP LAPCSRSTSE STAALGCLVK DYFPEPVTVS WNSGALTSGV HTFPAVLQSS | 180 |
| | GLYSLSSVVT VPSSSLGTKT YTCNVDHKPS NTKVDKRVES KYGPPCPPCP APEFLGGPSV | 240 |
| | FLFPPKPKDT LMISRTPEVT CVVVDVSQED PEVQFNWYVD GVEVHNAKTK PREEQFNSTY | 300 |
| | RVVSVITVLH QDWLNGKEYK CKVSNKGLPS SIEKTISKAK GQPREPQVYT LPPSQEEMTK | 360 |
| | NQVSLTCLVK GFYPSDIAVE WESNGQPENN YKTTPPVLDS DGSFFLYSRL TVDKSRWQEG | 420 |
| | NVFSCSVMHE ALHNHYTQKS LSLSLGK | 447 |
| SEQ ID NO: 474 | EIVLTQSPAT LSLSPGERAT LSCRASKGVS TSGYSYLHWY QQKPGQAPRL LIYLASYLES | 60 |
| pembrolizumab | GVPARFSGSG SGTDFTLTIS SLEPEDFAVY YCQHSRDLPL TFGGGTKVEI KRTVAAPSVF | 120 |
| light chain | IFPPSDEQLK SGTASVVCLL NNFYPREAKV QWKVDNALQS GNSQESVTEQ DSKDSTYSLS | 180 |
| | STLTLSKADY EKHKVYACEV THQGLSSPVT KSFNRGEC | 218 |
| SEQ ID NO: 475 | QVQLVQSGVE VKKPGASVKV SCKASGYTFT NYYMYWVRQA PGQGLEWMGG INPSNGGTNF | 60 |
| pembrolizumab | NEKFKNRVTL TTDSSTTTAY MELKSLQFDD TAVYYCARRD YRFDMGFDYW GQGTTVTVSS | 120 |
| variable heavy | | |
| chain | | |
| SEQ ID NO: 476 | EIVLTQSPAT LSLSPGERAT LSCRASKGVS TSGYSYLHWY QQKPGQAPRL LIYLASYLES | 60 |
| pembrolizumab | GVPARFSGSG SGTDFTLTIS SLEPEDFAVY YCQHSRDLPL TFGGGTKVEI K | 111 |
| variable light | | |
| chain | | |
| SEQ ID NO: 477 | NYYMY | 5 |
| pembrolizumab | | |
| heavy chain | | |
| CDR1 | | |
| SEQ ID NO: 478 | GINPSNGGTN FNEKFK | 16 |
| pembrolizumab | | |
| heavy chain | | |
| CDR2 | | |
| SEQ ID NO: 479 | RDYRFDMGFD Y | 11 |
| pembrolizumab | | |
| heavy chain | | |
| CDR3 | | |
| SEQ ID NO: 480 | RASKGVSTSG YSYLH | 15 |
| pembrolizumab | | |
| light chain | | |
| CDR1 | | |
| SEQ ID NO: 481 | LASYLES | 7 |
| pembrolizumab | | |
| light chain | | |
| CDR2 | | |
| SEQ ID NO: 482 | QHSRDLPLT | 9 |
| pembrolizumab | | |
| light chain | | |
| CDR3 | | |

In an embodiment, the PD-1 inhibitor is a commercially-available anti-PD-1 monoclonal antibody, such as anti-in-PD-1 clones J43 (Cat #BE0033-2) and RMP-14 (Cat #BE0146) (Bio X Cell, Inc., West Lebanon, NHT, USA). A number of commercially-available anti-PD-1 antibodies are known to one of ordinary skill in the art.

In an embodiment, the PD-1 inhibitor is an antibody disclosed in U.S. Pat. No. 8,354,509 or U.S. Patent Application Publication Nos. 2010/0266617 A1, 2013/0108651 A1, 2013/0109843 A2, the disclosures of which are incorporated by reference herein. In an embodiment, the PD-1 inhibitor is an anti-PD-1 antibody described in U.S. Pat. Nos. 8,287,856, 8,580,247, and 8,168,757 and U.S. Patent Application Publication Nos. 2009/0028857 A1, 2010/0285013 A1, 2013/0022600 A1, and 2011/0008369 A1, the teachings of which are hereby incorporated by reference. In another embodiment, the PD-1 inhibitor is an anti-PD-1 antibody disclosed in U.S. Pat. No. 8,735,553 Bi, the disclosure of which is incorporated herein by reference. In an embodiment, the PD-1 inhibitor is pidilizumab, also known as CT-011, which is described in U.S. Pat. No. 8,686,119, the disclosure of which is incorporated by reference herein.

In an embodiment, the PD-1 inhibitor may be a small molecule or a peptide, or a peptide derivative, such as those described in U.S. Pat. Nos. 8,907,053; 9,096,642; and 9,044,442 and U.S. Patent Application Publication No. US 2015/0087581; 1,2,4-oxadiazole compounds and derivatives such as those described in U.S. Patent Application Publication No. 2015/0073024; cyclic peptidomimetic compounds and derivatives such as those described in U.S. Patent Application Publication No. US 2015/0073042; cyclic compounds and derivatives such as those described in U.S. Patent Application Publication No. US 2015/0125491; 1,3,4-oxadiazole and 1,3,4-thiadiazole compounds and derivatives such as those described in International Patent Application Publication No. WO 2015/033301; peptide-based compounds and derivatives such as those described in International Patent Application Publication Nos. WO 2015/036927 and WO 2015/04490, or a macrocyclic peptide-based compounds and derivatives such as those described in U.S. Patent Application Publication No. US 2014/0294898; the disclosures of each of which are hereby incorporated by reference in their entireties.

In an embodiment, the PD-L1 or PD-L2 inhibitor may be any PD-L1 or PD-L2 inhibitor, antagonist, or blocker known in the art. In particular, it is one of the PD-L1 or PD-L2 inhibitors, antagonist, or blockers described in more detail in the following paragraphs. The terms “inhibitor,” “antagonist,” and “blocker” are used interchangeably herein in reference to PD-L1 and PD-L2 inhibitors. For avoidance of doubt, references herein to a PD-L1 or PD-L2 inhibitor that is an antibody may refer to a compound or antigen-binding fragments, variants, conjugates, or biosimilars thereof. For avoidance of doubt, references herein to a PD-L1 or PD-L2 inhibitor may refer to a compound or a pharmaceutically acceptable salt, ester, solvate, hydrate, cocrystal, or prodrug thereof.

In some embodiments, the compositions, processes and methods described herein include a PD-L1 or PD-L2 inhibitor. In some embodiments, the PD-L1 or PD-L2 inhibitor is a small molecule. In a preferred embodiment, the PD-L1 or PD-L2 inhibitor is an antibody (i.e., an anti-PD-1 antibody), a fragment thereof, including Fab fragments, or a single-chain variable fragment (scFv) thereof. In some embodiments the PD-L1 or PD-L2 inhibitor is a polyclonal antibody. In a preferred embodiment, the PD-L1 or PD-L2 inhibitor is a monoclonal antibody. In some embodiments, the PD-L1 or PD-L2 inhibitor competes for binding with PD-L1 or PD-L2, and/or binds to an epitope on PD-L1 or PD-L2. In an embodiment, the antibody competes for binding with PD-L1 or PD-L2, and/or binds to an epitope on PD-L1 or PD-L2.

In some embodiments, the PD-L1 inhibitors provided herein are selective for PD-L1, in that the compounds bind or interact with PD-L1 at substantially lower concentrations than they bind or interact with other receptors, including the PD-L2 receptor. In certain embodiments, the compounds bind to the PD-L1 receptor at a binding constant that is at least about a 2-fold higher concentration, about a 3-fold higher concentration, about a 5-fold higher concentration, about a 10-fold higher concentration, about a 20-fold higher concentration, about a 30-fold higher concentration, about a 50-fold higher concentration, about a 100-fold higher concentration, about a 200-fold higher concentration, about a 300-fold higher concentration, or about a 500-fold higher concentration than to the PD-L2 receptor.

In some embodiments, the PD-L2 inhibitors provided herein are selective for PD-L2, in that the compounds bind or interact with PD-L2 at substantially lower concentrations than they bind or interact with other receptors, including the PD-L1 receptor. In certain embodiments, the compounds bind to the PD-L2 receptor at a binding constant that is at least about a 2-fold higher concentration, about a 3-fold higher concentration, about a 5-fold higher concentration, about a 10-fold higher concentration, about a 20-fold higher concentration, about a 30-fold higher concentration, about a 50-fold higher concentration, about a 100-fold higher concentration, about a 200-fold higher concentration, about a 300-fold higher concentration, or about a 500-fold higher concentration than to the PD-L1 receptor.

Without being bound by any theory, it is believed that tumor cells express PD-L1, and that T cells express PD-1. However, PD-L1 expression by tumor cells is not required for efficacy of PD-1 or PD-L1 inhibitors or blockers. In an embodiment, the tumor cells express PD-L1. In another embodiment, the tumor cells do not express PD-L1. In some embodiments, the methods and compositions described herein include a combination of a PD-1 and a PD-L1 antibody, such as those described herein, in combination with a TIL. The administration of a combination of a PD-1 and a PD-L1 antibody and a TIL may be simultaneous or sequential.

In some embodiments, the compositions and methods described include a PD-L1 and/or PD-L2 inhibitor that binds human PD-L1 and/or PD-L2 with a K_{D} of about 100 pM or lower, binds human PD-L1 and/or PD-L2 with a K_{D} of about 90 pM or lower, binds human PD-L1 and/or PD-L2 with a K_{D} of about 80 pM or lower, binds human PD-L1 and/or PD-L2 with a K_{D} of about 70 pM or lower, binds human PD-L1 and/or PD-L2 with a K_{D} of about 60 pM or lower, a K_{D} of about 50 pM or lower, binds human PD-L1 and/or PD-L2 with a K_{D} of about 40 pM or lower, or binds human PD-L1 and/or PD-L2 with a K_{D} of about 30 pM or lower,

In some embodiments, the compositions and methods described include a PD-L1 and/or PD-L2 inhibitor that binds to human PD-L1 and/or PD-L2 with a k_{assoc} of about 7.5×10⁵ l/M·s or faster, binds to human PD-L1 and/or PD-L2 with a k_{assoc} of about 8×10⁵ l/M·s or faster, binds to human PD-L1 and/or PD-L2 with a k_{assoc} of about 8.5×10⁵ l/M·s or faster, binds to human PD-L1 and/or PD-L2 with a k_{assoc} of about 9×10⁵ l/M·s or faster, binds to human PD-L1 and/or PD-L2 with a k_{assoc} of about 9.5×10⁵ l/M·s and/or faster, or binds to human PD-L1 and/or PD-L2 with a k_{assoc} of about 1×10⁶ l/M·s or faster.

In some embodiments, the compositions and methods described include a PD-L1 and/or PD-L2 inhibitor that binds to human PD-L1 or PD-L2 with a k_{dissoc} of about 2×10⁻⁵ l/s or slower, binds to human PD-1 with a k_{dissoc} of about 2.1×10⁻⁵ l/s or slower, binds to human PD-1 with a k_{dissoc} of about 2.2×10⁻⁵ l/s or slower, binds to human PD-1 with a k_{dissoc} of about 2.3×10⁻⁵ l/s or slower, binds to human PD-1 with a k_{dissoc} of about 2.4×10⁻⁵ l/s or slower, binds to human PD-1 with a k_{dissoc} of about 2.5×10⁻⁵ l/s or slower, binds to human PD-1 with a k_{dissoc} of about 2.6×10⁻⁵ l/s or slower, binds to human PD-L1 or PD-L2 with a k_{dissoc} of about 2.7×10⁻⁵ l/s or slower, or binds to human PD-L1 or PD-L2 with a k_{dissoc} of about 3×10⁻⁵ l/s or slower.

In some embodiments, the compositions and methods described include a PD-L1 and/or PD-L2 inhibitor that blocks or inhibits binding of human PD-L1 or human PD-L2 to human PD-1 with an IC₅₀ of about 10 nM or lower; blocks or inhibits binding of human PD-L1 or human PD-L2 to human PD-1 with an IC₅₀ of about 9 nM or lower; blocks or inhibits binding of human PD-L1 or human PD-L2 to human PD-1 with an IC₅₀ of about 8 nM or lower; blocks or inhibits binding of human PD-L1 or human PD-L2 to human PD-1 with an IC₅₀ of about 7 nM or lower; blocks or inhibits binding of human PD-L1 or human PD-L2 to human PD-1 with an IC₅₀ of about 6 nM or lower; blocks or inhibits binding of human PD-L1 or human PD-L2 to human PD-1 with an IC₅₀ of about 5 nM or lower; blocks or inhibits binding of human PD-L1 or human PD-L2 to human PD-1 with an IC₅₀ of about 4 nM or lower; blocks or inhibits binding of human PD-L1 or human PD-L2 to human PD-1 with an IC₅₀ of about 3 nM or lower; blocks or inhibits binding of human PD-L1 or human PD-L2 to human PD-1 with an IC₅₀ of about 2 nM or lower; or blocks human PD-1, or blocks binding of human PD-L1 or human PD-L2 to human PD-1 with an IC₅₀ of about 1 nM or lower.

In an embodiment, the PD-L1 inhibitor is durvalumab, also known as MEDI4736 (which is commercially available from Medimmune, LLC, Gaithersburg, Maryland, a subsidiary of AstraZeneca plc.), or antigen-binding fragments, conjugates, or variants thereof. In an embodiment, the PD-L1 inhibitor is an antibody disclosed in U.S. Pat. No. 8,779,108 or U.S. Patent Application Publication No. 2013/0034559, the disclosures of which are incorporated by reference herein. The clinical efficacy of durvalumab has been described in Page, et al., Ann. Rev. Med., 2014, 65, 185-202; Brahmer, et al., J. Clin. Oncol. 2014, 32, 5s (supplement, abstract 8021); and McDermott, et al., Cancer Treatment Rev., 2014, 40, 1056-64. The preparation and properties of durvalumab are described in U.S. Pat. No. 8,779,108, the disclosure of which is incorporated by reference herein. The amino acid sequences of durvalumab are set forth in Table 50. The durvalumab monoclonal antibody includes disulfide linkages at 22-96, 22″-96″, 23′-89′, 23′″-89′″, 135′-195′, 135′″-195′″, 148-204, 148″-204″, 215′-224, 215′″-224″, 230-230″, 233-233″, 265-325, 265″-325″, 371-429, and 371″-429′; and N-glycosylation sites at Asn-301 and Asn-301″.

In an embodiment, a PD-L1 inhibitor comprises a heavy chain given by SEQ ID NO:483 and a light chain given by SEQ ID NO:484. In an embodiment, a PD-L1 inhibitor comprises heavy and light chains having the sequences shown in SEQ ID NO:483 and SEQ ID NO:484, respectively, or antigen binding fragments, Fab fragments, single-chain variable fragments (scFv), variants, or conjugates thereof. In an embodiment, a PD-L1 inhibitor comprises heavy and light chains that are each at least 99% identical to the sequences shown in SEQ ID NO:483 and SEQ ID NO:484, respectively. In an embodiment, a PD-L1 inhibitor comprises heavy and light chains that are each at least 98% identical to the sequences shown in SEQ ID NO:483 and SEQ ID NO:484, respectively. In an embodiment, a PD-L1 inhibitor comprises heavy and light chains that are each at least 97% identical to the sequences shown in SEQ ID NO:483 and SEQ ID NO:484, respectively. In an embodiment, a PD-L1 inhibitor comprises heavy and light chains that are each at least 96% identical to the sequences shown in SEQ ID NO:483 and SEQ ID NO:484, respectively. In an embodiment, a PD-L1 inhibitor comprises heavy and light chains that are each at least 95% identical to the sequences shown in SEQ ID NO:483 and SEQ ID NO:484, respectively.

In an embodiment, the PD-L1 inhibitor comprises the heavy and light chain CDRs or variable regions (VRs) of durvalumab. In an embodiment, the PD-L1 inhibitor heavy chain variable region (V_{H}) comprises the sequence shown in SEQ ID NO:485, and the PD-L1 inhibitor light chain variable region (V_{L}) comprises the sequence shown in SEQ ID NO:486, and conservative amino acid substitutions thereof. In an embodiment, a PD-L1 inhibitor comprises V_{H} and V_{L} regions that are each at least 99% identical to the sequences shown in SEQ ID NO:485 and SEQ ID NO:486, respectively. In an embodiment, a PD-L1 inhibitor comprises V_{H} and V_{L} regions that are each at least 98% identical to the sequences shown in SEQ ID NO:485 and SEQ ID NO:486, respectively. In an embodiment, a PD-L1 inhibitor comprises V_{H} and V_{L} regions that are each at least 97% identical to the sequences shown in SEQ ID NO:485 and SEQ ID NO:486, respectively. In an embodiment, a PD-L1 inhibitor comprises V_{H} and V_{L} regions that are each at least 96% identical to the sequences shown in SEQ ID NO:485 and SEQ ID NO:486, respectively. In an embodiment, a PD-L1 inhibitor comprises V_{H} and V_{L} regions that are each at least 95% identical to the sequences shown in SEQ ID NO:485 and SEQ ID NO:486, respectively.

In an embodiment, a PD-L1 inhibitor comprises heavy chain CDR1, CDR2 and CDR3 domains having the sequences set forth in SEQ ID NO:487, SEQ ID NO:488, and SEQ ID NO:489, respectively, and conservative amino acid substitutions thereof, and light chain CDR1, CDR2 and CDR3 domains having the sequences set forth in SEQ ID NO:490, SEQ ID NO:491, and SEQ ID NO:492, respectively, and conservative amino acid substitutions thereof. In an embodiment, the antibody competes for binding with, and/or binds to the same epitope on PD-L1 as any of the aforementioned antibodies.

In an embodiment, the PD-L1 inhibitor is an anti-PD-L1 biosimilar monoclonal antibody approved by drug regulatory authorities with reference to durvalumab. In an embodiment, the biosimilar comprises an anti-PD-L1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is durvalumab. In some embodiments, the one or more post-translational modifications are selected from one or more of: glycosylation, oxidation, deamidation, and truncation. In some embodiments, the biosimilar is an anti-PD-L1 antibody authorized or submitted for authorization, wherein the anti-PD-L1 antibody is provided in a formulation which differs from the formulations of a reference medicinal product or reference biological product, wherein the reference medicinal product or reference biological product is durvalumab. The anti-PD-L1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is durvalumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is durvalumab.

TABLE 50
Amino acid sequences for PD-L1 inhibitors related to durvalumab.

| Identifier | Sequence (One-Letter Amino Acid Symbols) | |
|---|---|---|
| SEQ ID NO: 483 | EVQLVESGGG LVQPGGSLRL SCAASGFTFS RYWMSWVRQA PGKGLEWVAN IKQDGSEKYY | 60 |
| durvalumab | VDSVKGRFTI SRDNAKNSLY LQMNSLRAED TAVYYCAREG GWFGELAFDY WGQGTLVTVS | 120 |
| heavy chain | SASTKGPSVF PLAPSSKSTS GGTAALGCLV KDYFPEPVTV SWNSGALTSG VHTFPAVLQS | 180 |
| | SGLYSLSSVV TVPSSSLGTQ TYICNVNHKP SNTKVDKRVE PKSCDKTHTC PPCPAPEFEG | 240 |
| | GPSVFLFPPK PKDTLMISRT PEVTCVVVDV SHEDPEVKFN WYVDGVEVHN AKTKPREEQY | 300 |
| | NSTYRVVSVL TVLHQDWLNG KEYKCKVSNK ALPASIEKTI SKAKGQPREP QVYTLPPSRE | 360 |
| | EMTKNQVSLT CLVKGFYPSD IAVEWESNGQ PENNYKTTPP VLDSDGSFFL YSKLTVDKSR | 420 |
| | WQQGNVFSCS VMHEALHNHY TQKSLSLSPG K | 451 |
| SEQ ID NO: 484 | EVQLVESGGG LVQPGGSLRL SCAASGFTFS RYWMSWVRQA PGKGLEWVAN EIVLTQSPGT | 60 |
| durvalumab | LSLSPGERAT LSCRASQRVS SSYLAWYQQK PGQAPRLLIY DASSRATGIP DRFSGSGSGT | 120 |
| light chain | DFTLTISRLE PEDFAVYYCQ QYGSLPWTFG QGTKVEIKRT VAAPSVFIFP PSDEQLKSGT | 180 |
| | ASVVCLLNNF YPREAKVQWK VDNALQSGNS QESVTEQDSK DSTYSLSSTL TLSKADYEKH | 240 |
| | KVYACEVTHQ GLSSPVTKSF NRGEC | 265 |
| SEQ ID NO: 485 | EVQLVESGGG LVQPGGSLRL SCAASGFTFS RYWMSWVRQA PGKGLEWVAN IKQDGSEKYY | 60 |
| durvalumab | VDSVKGRFTI SRDNAKNSLY LQMNSLRAED TAVYYCAREG GWFGELAFDY WGQGTLVTVS | 120 |
| variable | S | 121 |
| heavy chain | | |
| SEQ ID NO: 486 | EIVLTQSPGT LSLSPGERAT LSCRASQRVS SSYLAWYQQK PGQAPRLLIY DASSRATGIP | 60 |
| durvalumab | DRFSGSGSGT DFTLTISRLE PEDFAVYYCQ QYGSLPWTFG QGTKVEIK | 108 |
| variable | | |
| light chain | | |
| SEQ ID NO: 487 | RYWMS | 5 |
| durvalumab | | |
| heavy chain | | |
| CDR1 | | |
| SEQ ID NO: 488 | NIKQDGSEKY YVDSVKG | 17 |
| durvalumab | | |
| heavy chain | | |
| CDR2 | | |
| SEQ ID NO: 489 | EGGWFGELAF DY | 12 |
| durvalumab | | |
| heavy chain | | |
| CDR3 | | |
| SEQ ID NO: 490 | RASQRVSSSY LA | 12 |
| durvalumab | | |
| light chain | | |
| CDR1 | | |
| SEQ ID NO: 491 | DASSRAT | 7 |
| durvalumab | | |
| light chain | | |
| CDR2 | | |
| SEQ ID NO: 492 | QQYGSLPWT | 9 |
| durvalumab | | |
| light chain | | |
| CDR3 | | |

In an embodiment, the PD-L1 inhibitor is avelumab, also known as MSB0010718C (commercially available from Merck KGaA/EMD Serono), or antigen-binding fragments, conjugates, or variants thereof. The preparation and properties of avelumab are described in U.S. Patent Application Publication No. US 2014/0341917 A1, the disclosure of which is specifically incorporated by reference herein. The amino acid sequences of avelumab are set forth in Table 51. Avelumab has intra-heavy chain disulfide linkages (C23-C104) at 22-96, 147-203, 264-324, 370-428, 22″-96″, 147-203″, 264″-324″, and 370″-428″; intra-light chain disulfide linkages (C23-C104) at 22′-90′, 138′-197′, 22″-90′″, and 138″-197′″; intra-heavy-light chain disulfide linkages (h 5-C_{L} 126) at 223-215′ and 223″-215′″; intra-heavy-heavy chain disulfide linkages (h 11, h 14) at 229-229″ and 232-232″; N-glycosylation sites (H CH₂ N84.4) at 300, 300″; fucosylated complex bi-antennary CHO-type glycans; and H CUS K2 C-terminal lysine clipping at 450 and 450′.

In an embodiment, a PD-L1 inhibitor comprises a heavy chain given by SEQ ID NO:493 and a light chain given by SEQ ID NO:494. In an embodiment, a PD-L1 inhibitor comprises heavy and light chains having the sequences shown in SEQ ID NO:493 and SEQ ID NO:494, respectively, or antigen binding fragments, Fab fragments, single-chain variable fragments (scFv), variants, or conjugates thereof. In an embodiment, a PD-L1 inhibitor comprises heavy and light chains that are each at least 99% identical to the sequences shown in SEQ ID NO:493 and SEQ ID NO:494, respectively. In an embodiment, a PD-L1 inhibitor comprises heavy and light chains that are each at least 98% identical to the sequences shown in SEQ ID NO:493 and SEQ ID NO:494, respectively. In an embodiment, a PD-L1 inhibitor comprises heavy and light chains that are each at least 97% identical to the sequences shown in SEQ ID NO:493 and SEQ ID NO:494, respectively. In an embodiment, a PD-L1 inhibitor comprises heavy and light chains that are each at least 96% identical to the sequences shown in SEQ ID NO:493 and SEQ ID NO:494, respectively. In an embodiment, a PD-L1 inhibitor comprises heavy and light chains that are each at least 95% identical to the sequences shown in SEQ ID NO:493 and SEQ ID NO:494, respectively.

In an embodiment, the PD-L1 inhibitor comprises the heavy and light chain CDRs or variable regions (VRs) of avelumab. In an embodiment, the PD-L1 inhibitor heavy chain variable region (V_{H}) comprises the sequence shown in SEQ ID NO:495, and the PD-L1 inhibitor light chain variable region (V_{L}) comprises the sequence shown in SEQ ID NO:496, and conservative amino acid substitutions thereof. In an embodiment, a PD-L1 inhibitor comprises V_{H} and V_{L} regions that are each at least 99% identical to the sequences shown in SEQ ID NO:495 and SEQ ID NO:496, respectively. In an embodiment, a PD-L1 inhibitor comprises V_{H} and V_{L} regions that are each at least 98% identical to the sequences shown in SEQ ID NO:496 and SEQ ID NO:496, respectively. In an embodiment, a PD-L1 inhibitor comprises V_{H} and V_{L} regions that are each at least 97% identical to the sequences shown in SEQ ID NO:495 and SEQ ID NO:496, respectively. In an embodiment, a PD-L1 inhibitor comprises V_{H} and V_{L} regions that are each at least 96% identical to the sequences shown in SEQ ID NO:495 and SEQ ID NO:496, respectively. In an embodiment, a PD-L1 inhibitor comprises V_{H} and V_{L} regions that are each at least 95% identical to the sequences shown in SEQ ID NO:495 and SEQ ID NO:496, respectively.

In an embodiment, a PD-L1 inhibitor comprises heavy chain CDR1, CDR2 and CDR3 domains having the sequences set forth in SEQ ID NO:497, SEQ ID NO:498, and SEQ ID NO:499, respectively, and conservative amino acid substitutions thereof, and light chain CDR1, CDR2 and CDR3 domains having the sequences set forth in SEQ ID NO:500, SEQ ID NO:501, and SEQ ID NO:502, respectively, and conservative amino acid substitutions thereof. In an embodiment, the antibody competes for binding with, and/or binds to the same epitope on PD-L1 as any of the aforementioned antibodies.

In an embodiment, the PD-L1 inhibitor is an anti-PD-L1 biosimilar monoclonal antibody approved by drug regulatory authorities with reference to avelumab. In an embodiment, the biosimilar comprises an anti-PD-L1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is avelumab. In some embodiments, the one or more post-translational modifications are selected from one or more of: glycosylation, oxidation, deamidation, and truncation. In some embodiments, the biosimilar is an anti-PD-L1 antibody authorized or submitted for authorization, wherein the anti-PD-L1 antibody is provided in a formulation which differs from the formulations of a reference medicinal product or reference biological product, wherein the reference medicinal product or reference biological product is avelumab. The anti-PD-L1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is avelumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is avelumab.

TABLE 51
Amino acid sequences for PD-L1 inhibitors related to avelumab.

| Identifier | Sequence (One-Letter Amino Acid Symbols) | |
|---|---|---|
| SEQ ID NO: 493 | EVQLLESGGG LVQPGGSLRL SCAASGFTFS SYIMMWVRQA PGKGLEWVSS IYPSGGITFY | 60 |
| avelumab | ADTVKGRFTI SRDNSKNTLY LQMNSLRAED TAVYYCARIK LGTVTTVDYW GQGTLVTVSS | 120 |
| heavy chain | ASTKGPSVFP LAPSSKSTSG GTAALGCLVK DYFPEPVTVS WNSGALTSGV HTFPAVLQSS | 180 |
| | GLYSLSSVVT VPSSSLGTQT YICNVNHKPS NTKVDKKVEP KSCDKTHTCP PCPAPELLGG | 240 |
| | PSVFLFPPKP KDTLMISRTP EVTCVVVDVS HEDPEVKFNW YVDGVEVHNA KTKPREEQYN | 300 |
| | STYRVVSVLT VLHQDWLNGK EYKCKVSNKA LPAPIEKTIS KAKGQPREPQ VYTLPPSRDE | 360 |
| | LTKNQVSLTC LVKGFYPSDI AVEWESNGQP ENNYKTTPPV LDSDGSFFLY SKLTVDKSRW | 420 |
| | QQGNVFSCSV MHEALHNHYT QKSLSLSPGK | 450 |
| SEQ ID NO: 494 | QSALTQPASV SGSPGQSITI SCTGTSSDVG GYNYVSWYQQ HPGKAPKLMI YDVSNRPSGV | 60 |
| avelumab | SNRFSGSKSG NTASLTISGL QAEDEADYYC SSYTSSSTRV FGTGTKVTVI GQPKANPTVT | 120 |
| light chain | LFPPSSEELQ ANKATLVCLI SDFYPGAVTV AWKADGSPVK AGVETTKPSK QSNNKYAASS | 180 |
| | YLSLTPEQWK SHRSYSCQVT HEGSTVEKTV APTECS | 216 |
| SEQ ID NO: 495 | EVQLLESGGG LVQPGGSLRL SCAASGFTFS SYIMMWVRQA PGKGLEWVSS IYPSGGITFY | 60 |
| avelumab | ADTVKGRFTI SRDNSKNTLY LQMNSLRAED TAVYYCARIK LGTVTTVDYW GQGTLVTVSS | 120 |
| variable | | |
| heavy chain | | |
| SEQ ID NO: 496 | QSALTQPASV SGSPGQSITI SCTGTSSDVG GYNYVSWYQQ HPGKAPKLMI YDVSNRPSGV | 60 |
| avelumab | SNRFSGSKSG NTASLTISGL QAEDEADYYC SSYTSSSTRV FGTGTKVTVL | 110 |
| variable | | |
| light chain | | |
| SEQ ID NO: 497 | SYIMM | 5 |
| avelumab | | |
| heavy chain | | |
| CDR1 | | |
| SEQ ID NO: 498 | SIYPSGGITF YADTVKG | 17 |
| avelumab | | |
| heavy chain | | |
| CDR2 | | |
| SEQ ID NO: 499 | IKLGTVTTVD Y | 11 |
| avelumab | | |
| heavy chain | | |
| CDR3 | | |
| SEQ ID NO: 500 | TGTSSDVGGY NYVS | 14 |
| avelumab | | |
| light chain | | |
| CDR1 | | |
| SEQ ID NO: 501 | DVSNRPS | 7 |
| avelumab | | |
| light chain | | |
| CDR2 | | |
| SEQ ID NO: 502 | SSYTSSSTRV | 10 |
| avelumab | | |
| light chain | | |
| CDR3 | | |

In an embodiment, the PD-L1 inhibitor is atezolizumab, also known as MPDL3280A or RG7446 (commercially available as TECENTRIQ from Genentech, Inc., a subsidiary of Roche Holding AG, Basel, Switzerland), or antigen-binding fragments, conjugates, or variants thereof. In an embodiment, the PD-L1 inhibitor is an antibody disclosed in U.S. Pat. No. 8,217,149, the disclosure of which is specifically incorporated by reference herein. In an embodiment, the PD-L1 inhibitor is an antibody disclosed in U.S. Patent Application Publication Nos. 2010/0203056 A1, 2013/0045200 A1, 2013/0045201 A1, 2013/0045202 A1, or 2014/0065135 A1, the disclosures of which are specifically incorporated by reference herein. The preparation and properties of atezolizumab are described in U.S. Pat. No. 8,217,149, the disclosure of which is incorporated by reference herein. The amino acid sequences of atezolizumab are set forth in Table 52. Atezolizumab has intra-heavy chain disulfide linkages (C23-C104) at 22-96, 145-201, 262-322, 368-426, 22″-96″, 145″-201″, 262″-322″, and 368″-426″; intra-light chain disulfide linkages (C23-C104) at 23′-88′, 134′-194′, 23′″-88′″, and 134′″-194′″; intra-heavy-light chain disulfide linkages (h 5-C_{L} 126) at 221-214′ and 221″-214′″; intra-heavy-heavy chain disulfide linkages (h 11, h 14) at 227-227″ and 230-230″; and N-glycosylation sites (H CH₂ N84.4>A) at 298 and 298′.

In an embodiment, a PD-L1 inhibitor comprises a heavy chain given by SEQ ID NO:503 and a light chain given by SEQ ID NO:504. In an embodiment, a PD-L1 inhibitor comprises heavy and light chains having the sequences shown in SEQ ID NO:503 and SEQ ID NO:504, respectively, or antigen binding fragments, Fab fragments, single-chain variable fragments (scFv), variants, or conjugates thereof. In an embodiment, a PD-L1 inhibitor comprises heavy and light chains that are each at least 99% identical to the sequences shown in SEQ ID NO:503 and SEQ ID NO:504, respectively. In an embodiment, a PD-L1 inhibitor comprises heavy and light chains that are each at least 98% identical to the sequences shown in SEQ ID NO:503 and SEQ ID NO:504, respectively. In an embodiment, a PD-L1 inhibitor comprises heavy and light chains that are each at least 97% identical to the sequences shown in SEQ ID NO:503 and SEQ ID NO:504, respectively. In an embodiment, a PD-L1 inhibitor comprises heavy and light chains that are each at least 96% identical to the sequences shown in SEQ ID NO:503 and SEQ ID NO:504, respectively. In an embodiment, a PD-L1 inhibitor comprises heavy and light chains that are each at least 95% identical to the sequences shown in SEQ ID NO:503 and SEQ ID NO:504, respectively.

In an embodiment, the PD-L1 inhibitor comprises the heavy and light chain CDRs or variable regions (VRs) of atezolizumab. In an embodiment, the PD-L1 inhibitor heavy chain variable region (V_{H}) comprises the sequence shown in SEQ ID NO:505, and the PD-L1 inhibitor light chain variable region (V_{L}) comprises the sequence shown in SEQ ID NO:506, and conservative amino acid substitutions thereof. In an embodiment, a PD-L1 inhibitor comprises V_{H} and V_{L} regions that are each at least 99% identical to the sequences shown in SEQ ID NO:505 and SEQ ID NO:506, respectively. In an embodiment, a PD-L1 inhibitor comprises V_{H} and V_{L} regions that are each at least 98% identical to the sequences shown in SEQ ID NO:505 and SEQ ID NO:506, respectively. In an embodiment, a PD-L1 inhibitor comprises V_{H} and V_{L} regions that are each at least 97% identical to the sequences shown in SEQ ID NO:505 and SEQ ID NO:506, respectively. In an embodiment, a PD-L1 inhibitor comprises V_{H} and V_{L} regions that are each at least 96% identical to the sequences shown in SEQ ID NO:505 and SEQ ID NO:506, respectively. In an embodiment, a PD-L1 inhibitor comprises V_{H} and V_{L} regions that are each at least 95% identical to the sequences shown in SEQ ID NO:505 and SEQ ID NO:506, respectively.

In an embodiment, a PD-L1 inhibitor comprises heavy chain CDR1, CDR2 and CDR3 domains having the sequences set forth in SEQ ID NO:507, SEQ ID NO:508, and SEQ ID NO:509, respectively, and conservative amino acid substitutions thereof, and light chain CDR1, CDR2 and CDR3 domains having the sequences set forth in SEQ ID NO:510, SEQ ID NO:511, and SEQ ID NO:512, respectively, and conservative amino acid substitutions thereof. In an embodiment, the antibody competes for binding with, and/or binds to the same epitope on PD-L1 as any of the aforementioned antibodies.

In an embodiment, the anti-PD-L1 antibody is an anti-PD-L1 biosimilar monoclonal antibody approved by drug regulatory authorities with reference to atezolizumab. In an embodiment, the biosimilar comprises an anti-PD-L1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is atezolizumab. In some embodiments, the one or more post-translational modifications are selected from one or more of: glycosylation, oxidation, deamidation, and truncation. In some embodiments, the biosimilar is an anti-PD-L1 antibody authorized or submitted for authorization, wherein the anti-PD-L1 antibody is provided in a formulation which differs from the formulations of a reference medicinal product or reference biological product, wherein the reference medicinal product or reference biological product is atezolizumab. The anti-PD-L1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is atezolizumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is atezolizumab.

TABLE 52
Amino acid sequences for PD-L1 inhibitors related to atezolizumab.

| Identifier | Sequence (One-Letter Amino Acid Symbols) | |
|---|---|---|
| SEQ ID NO: 503 | EVQLVESGGG LVQPGGSLRL SCAASGFTFS DSWIHWVRQA PGKGLEWVAW ISPYGGSTYY | 60 |
| atezolizumab | ADSVKGRFTI SADTSKNTAY LQMNSLRAED TAVYYCARRH WPGGFDYWGQ GTLVTVSSAS | 120 |
| heavy chain | TKGPSVFPLA PSSKSTSGGT AALGCLVKDY FPEPVTVSWN SGALTSGVHT FPAVLQSSGL | 180 |
| | YSLSSVVTVP SSSLGTQTYI CNVNHKPSNT KVDKKVEPKS CDKTHTCPPC PAPELLGGPS | 240 |
| | VFLFPPKPKD TLMISRTPEV TCVVVDVSHE DPEVKFNWYV DGVEVHNAKT KPREEQYAST | 300 |
| | YRVVSVLTVL HQDWLNGKEY KCKVSNKALP APIEKTISKA KGQPREPQVY TLPPSREEMT | 360 |
| | KNQVSLTCLV KGFYPSDIAV EWESNGQPEN NYKTTPPVLD SDGSFFLYSK LTVDKSRWQQ | 420 |
| | GNVFSCSVMH EALHNHYTQK SLSLSPGK | 448 |
| SEQ ID NO: 504 | DIQMTQSPSS LSASVGDRVT ITCRASQDVS TAVAWYQQKP GKAPKLLIYS ASFLYSGVPS | 60 |
| atezolizumab | RFSGSGSGTD FTLTISSLQP EDFATYYCQQ YLYHPATFGQ GTKVEIKRTV AAPSVFIFPP | 120 |
| light chain | SDEQLKSGTA SVVCLLNNFY PREAKVQWKV DNALQSGNSQ ESVTEQDSKD STYSLSSTLT | 180 |
| | LSKADYEKHK VYACEVTHQG LSSPVTKSFN RGEC | 214 |
| SEQ ID NO: 505 | EVQLVESGGG LVQPGGSLRL SCAASGFTFS DSWIHWVRQA PGKGLEWVAW ISPYGGSTYY | 60 |
| atezolizumab | ADSVKGRFTI SADTSKNTAY LQMNSLRAED TAVYYCARRH WPGGFDYWGQ GTLVTVSA | 118 |
| variable | | |
| heavy chain | | |
| SEQ ID NO: 506 | DIQMTQSPSS LSASVGDRVT ITCRASQDVS TAVAWYQQKP GKAPKLLIYS ASFLYSGVPS | 60 |
| atezolizumab | RFSGSGSGTD FTLTISSLQP EDFATYYCQQ YLYHPATFGQ GTKVEIKR | 108 |
| variable | | |
| light chain | | |
| SEQ ID NO: 507 | GFTFSDSWIH | 10 |
| atezolizumab | | |
| heavy chain | | |
| CDR1 | | |
| SEQ ID NO: 508 | AWISPYGGST YYADSVKG | 18 |
| atezolizumab | | |
| heavy chain | | |
| CDR2 | | |
| SEQ ID NO: 509 | RHWPGGFDY | 9 |
| atezolizumab | | |
| heavy chain | | |
| CDR3 | | |
| SEQ ID NO: 510 | RASQDVSTAV A | 11 |
| atezolizumab | | |
| light chain | | |
| CDR1 | | |
| SEQ ID NO: 511 | SASFLYS | 7 |
| atezolizumab | | |
| light chain | | |
| CDR2 | | |
| SEQ ID NO: 512 | QQYLYHPAT | 9 |
| atezolizumab | | |
| light chain | | |
| CDR3 | | |

In an embodiment, PD-L1 inhibitors include those antibodies described in U.S. Patent Application Publication No. US 2014/0341917 A1, the disclosure of which is incorporated by reference herein. In another embodiment, antibodies that compete with any of these antibodies for binding to PD-L1 are also included. In an embodiment, the anti-PD-L1 antibody is MDX-1105, also known as BMS-935559, which is disclosed in U.S. Pat. No. 7,943,743, the disclosures of which are incorporated by reference herein. In an embodiment, the anti-PD-L1 antibody is selected from the anti-PD-L1 antibodies disclosed in U.S. Pat. No. 7,943,743, which are incorporated by reference herein.

In an embodiment, the PD-L1 inhibitor is a commercially-available monoclonal antibody, such as INVIVOMAB anti-m-PD-L1 clone 10F.9G2 (Catalog #BE0101, Bio X Cell, Inc., West Lebanon, NH, USA). In an embodiment, the anti-PD-L1 antibody is a commercially-available monoclonal antibody, such as AFFYMETRIX EBIOSCIENCE (MIH1). A number of commercially-available anti-PD-L1 antibodies are known to one of ordinary skill in the art.

In an embodiment, the PD-L2 inhibitor is a commercially-available monoclonal antibody, such as BIOLEGEND 24F.10C12 Mouse IgG2a, κ isotype (catalog #329602 Biolegend, Inc., San Diego, CA), SIGMA anti-PD-L2 antibody (catalog #SAB3500395, Sigma-Aldrich Co., St. Louis, MO), or other commercially-available anti-PD-L2 antibodies known to one of ordinary skill in the art.

While preferred embodiments of the invention are shown and described herein, such embodiments are provided by way of example only and are not intended to otherwise limit the scope of the invention. Various alternatives to the described embodiments of the invention may be employed in practicing the invention.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1—Methods of Expanding TILs and Treating Cancer with Expanded TILs

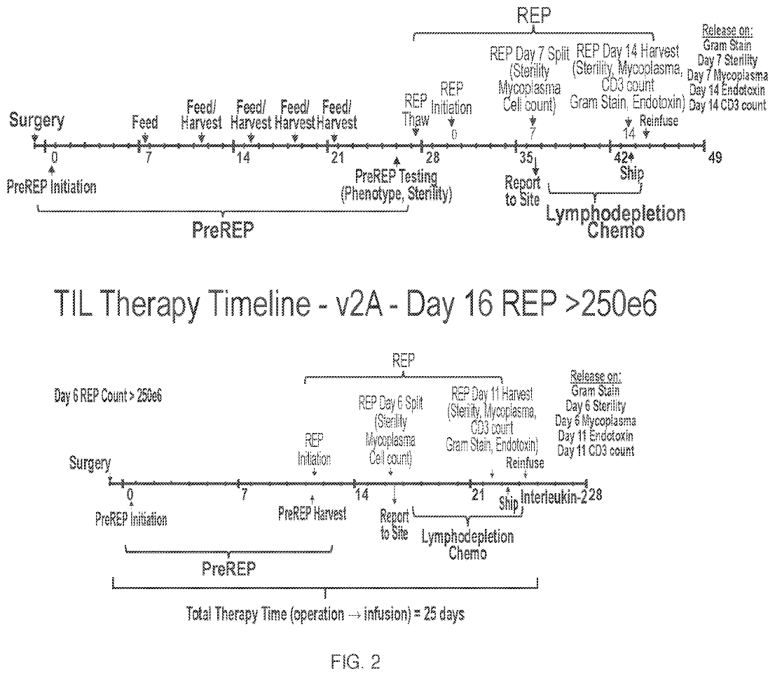

TILs may be expanded using methods known in the art and any method described herein. For example, methods for expanding TILs are depicted in FIG. 1. A TNFRSF agonist may be added to the method of FIG. 1 as described herein. The TNFRSF agonist may be, for example, a 4-1BB or an OX40 agonist, and may be added during the pre-REP or the REP phases, or during both phases, at concentrations sufficient to enhance TIL growth. The expansion of TILs may be further combined with any method of treating cancer in combination with a TNFRSF agonist in a patient described herein. Methods for expanding TILs and treating a cancer patient with expanded TILs are shown in FIG. 2.

### Example 2—Methods of Expanding TILs Using 4-1BB Agonists

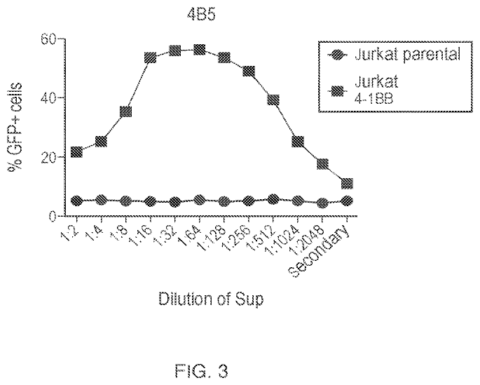

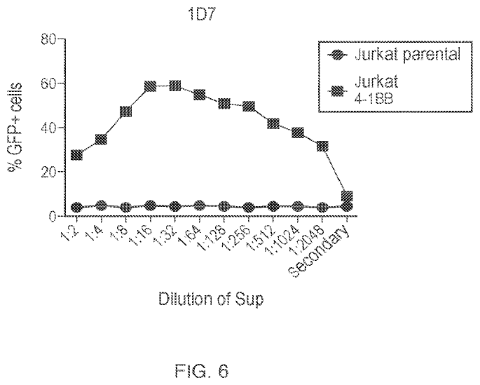

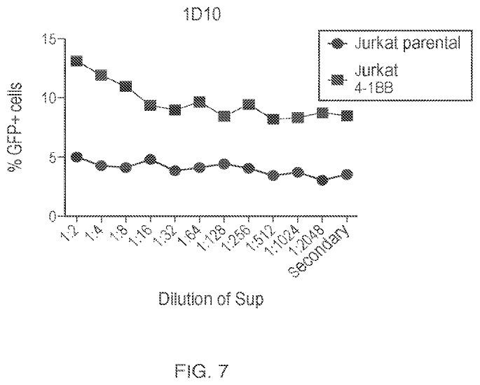

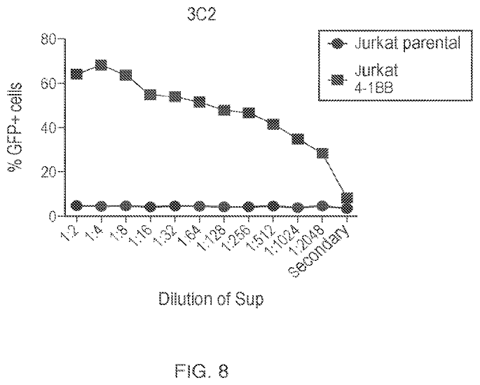

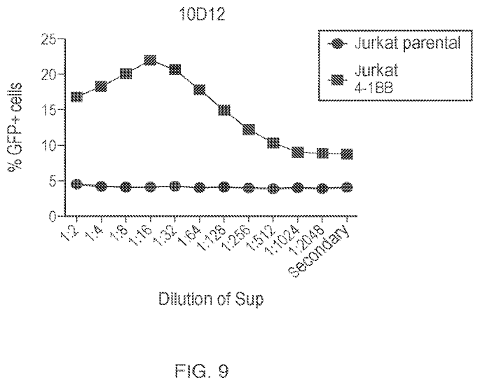

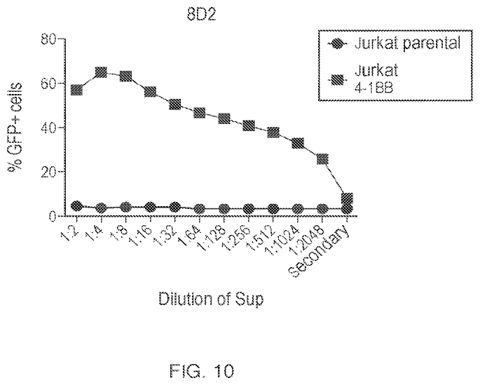

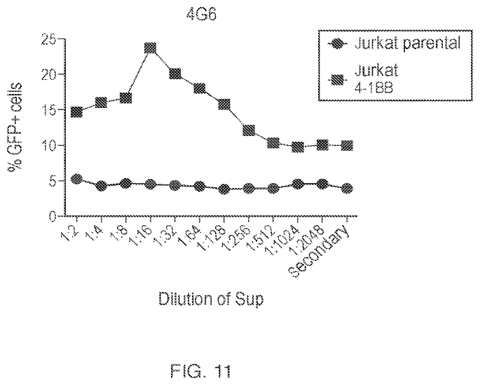

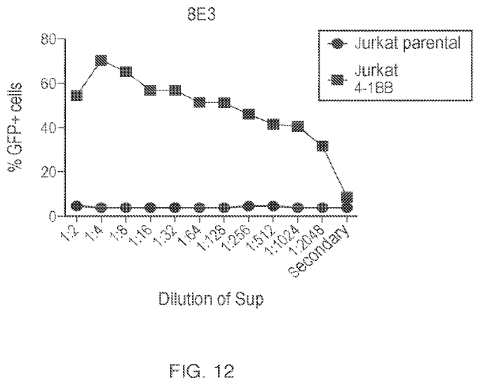

4-1BB-Fc hybridomas were produced and screened for their ability to activate the 4-1BB signaling pathway by cross-linking with secondary antibody. Hybridoma supernatants were evaluated for their ability to activate 4-1BB signaling on Jurkat cells expressing NF-kB using a green fluorescent protein (GFP) reporter in a dose dependent manner. Supernatants were incubated with Jurkat cells for 20 minutes at room temperature followed by crosslinking with goat anti-mouse secondary antibody (1 mg/mL) overnight at 37° C. Results are shown in FIG. 3 to FIG. 12 for ten clones identified that bound to 4-1BB on Jurkat cells.

Cells were analyzed on Intellicyte for NKkB Pathway activation revealed by GFP reporter expression. The results indicate that the antibodies activate the 4-1BB pathway on Jurkat cells without secondary antibody crosslinking.

### Example 3—Methods of Expanding TILs from Ex-Vivo Cultured Solid Tumor Fragments (Multiple Histologies) Using Utomilumab or Urelumab and 11D4/18D8 and Effect of Activation with Antibodies to 4-1BB and/or OX40 on Expansion and Function of TILs

TILs are primarily antigen experienced (non-naïve) T cells found to varying degrees in all adult tumors associated with immunosuppressive microenvironments in which the local accumulation of damage associated molecular pattern molecules (DAMPs) as well as induced checkpoint receptors including CTLA-4 and PD-1 have often been engaged. Chacon, et al., Clin. Cancer Res. 2015, 21, 611-21; Joseph, et al., Clin. Cancer Res. 2011, 17, 4882-91. These markers, as well as TIM3, LAG3, and TIGIT, define an exhausted phenotype. As such, TIL-expressed co-stimulatory receptors modify TIL fate and expansion. Activation of 4-1BB and or OX40 on TILs enables expansion of TILs from tumor fragments beyond that achievable with IL-2 alone. Activation of other co-stimulatory receptors and/or antagonism of checkpoint receptors will further enhance TIL function (survival, circumvention of tumor immunosuppression), emigration from tumor fragments, and promote in-vitro expansion. Furthermore, these studies in vitro may predict responsiveness to in vivo application of these antibodies alone or in combination with adoptive transfer of TILs. Immunomodulatory mAbs specific for these two activating co-stimulator molecules (e.g., OX40, 11D4 or 18D8, and 4-1BB, utomilumab or urelumab) can be tested for such capacity. It is hypothesized that activation of the costimulator receptors, 4-1BB and OX40, within tumor fragments enhances TIL emigration from fragments of tumor, proliferation, promotion of a memory phenotype and cytotoxicity of emergent T cells. The main goal of this study is to determine whether mAbs specific for (a) 4-1BB and OX40 in combination or (b) anti-4-1BB and (c) anti-OX40 alone augments the outgrowth of cytotoxic and memory phenotype of TIL from tumor fragments.

15 mg of each purified mAb specific for (a) OX40 and (b) 4-1BB is used. Tumors of various histologies may be obtained from commercial sources. In total, 20 independent patient tumors will be obtained. Tumors will be shipped in sterile HBSS or another appropriate medium. The tumors will be handled only in a laminar flow hood to maintain sterile conditions. When possible (if tumor>0.5 cm in diameter), a portion of the tumor will be processed for FFPE and/or cryopreserved for downstream IHC and/or DNA/RNA isolation. Biomarker analysis via IHC will include CD3, CD11c, and PD1 and PD-L1. Whenever possible, autologous blood samples (up to 20 mL) will be acquired and PBMCs will be cryopreserved. If whole exome sequencing is performed on the tumors, exome sequences from banked autologous PBMCs will be defined as normal (i.e., no mutations). Alternatively, tumor single cell suspensions may be utilized. The tumors will be washed after receipt and divided into 2-3 mm fragments and placed into cell culture into 24-well plates (1 fragment per well) or 6-well plates (4 fragments per well) with culture medium supplemented with 6,000 IU/mL IL-2 (recombinant) only, OX40 agonist, anti-4-1BB agonist, and a combination of OX40 and 4-1BB agonists in triplicates. In some experiments where sufficient tumor is available, titrations of IL-2 (6,000; 600; 60; and 0 IU of IL-2) will be tested. An excipient control for the IL-2 will be used. The final concentration of each mAb will be 30 μg/mL. Following 24-48 hours of culture, 250 μL of supernatant will be collected from each condition and stored at −20° C. for subsequent analysis of cytokine and chemokine concentrations (pg/10⁶ cell/24 hours). TILs will be collected from each condition on day 11, day 21 and/or day of the ‘pre-REP’ (at least 500,000 cells per sample). Two aliquots of TILs will be pelleted and resuspended in <10 μL of PBS and will be frozen in −80 C. If less than <10⁶ cells are collected, only gene expression arrays will be performed. Cultures will be fed on day 7 by partial removal of “spent” medium and addition of an equal volume of culture medium plus 6000 IU/mL IL-2. The spent medium will be stored at −20° C. for subsequent cytokine/chemokine analysis using a multiplex assay (e.g., Luminex 100 system). Additional mAb will be added to the culture on day 7 if sufficient tumor fragments are available for initiation of more than 1 replicate of experimental conditions. TIL cultures will be maintained for an additional 14 days. On day 21, the total cell yield, viability, cell surface and intracellular immunophenotype will be determined using flow cytometry. The following markers will be included: CD45RA, CCR7, CD3, TCR-alpha/beta, CD4, CD8, CXCR3, CD56, CD27, CD28, PD-1, PD-L1, BTLA, KLRG1, CD137, CD134, CD33, CD57, CD25, CD127, TIM-3, LAG-3, TIGIT, RAGE, and Ki67. Other biomarkers including CD107a, NKG2D, KIRS, chemokine death receptors (Fas, DR4) and anti-apoptotic/pro-autophagic proteins (BCL-2, BCL-XL, Bim, CD200, and LC3/HMGB1) will also be assessed if sufficient cells are available. Intracellular markers of cytotoxicity and regulatory T cells, Granzyme B, pSTAT3, pSTAT1, and FOXP3, respectively will be assessed. Lytic potency of TILs will be determined using a lysis assay. In cases where additional tumor material is available and (a) a tumor cell suspension generated following enzymatic digestion, (b) an autologous tumor line generated from aforementioned tumor and/or (c) homologous cell line (if available) will be co-cultured with harvested TIL and IFN-γ release measured. If excess cells are obtained, these will be cryopreserved for isolating RNA and DNA for gene expression analysis (including TCR Vβ clonotyping analysis) which can be performed at a later time using an extended budget. If efficacy (defined below) is observed with anti PDL-1 and anti-CTLA-4 or the combination thereof, the possibility of lowering the concentration of indicated mAb(s) in tumor fragment cultures or performing a more detailed dose response assessment will be explored. If tumor fragments are visible in culture on day 7, they will be harvested and if sufficient cells are available after generation of a single cell suspension, they will be subjected to genetic analysis and flow cytometric phenotypic analysis (in that priority; to be negotiated). Flow cytometric analysis will focus on the phenotype of T cells, dendritic cells, macrophages, B cells, and NK cells after staining using appropriate fluorescent mAb panels. The markers will include: CD11c, CD11b, HLA class II, CD80, CD86, CD83, CD56, CD16, CD19, and CD20.

The criteria used to assess the efficacy of the addition of 4-1BB agonist, OX40 agonist, and the combination thereof to the tumor fragment cultures are summarized as follows:
Increased number of TIL following expansion (CD4 and/or CD8)
Decreased number of Treg following expansion
Changes in T-cell proliferation markers (Ki67) in effector and Tregs
Changes in effector/memory/differentiated phenotype, CD27, CD28, CD57, CD45RA, HLA-DR, CCR7, OX40, ICOS, CD45RA; telomerase length))
Increased NK cell (CD3⁻/CD56⁺) numbers, proliferation and activation status
Exploratory changes in intracellular signaling protein or phosphoprotein levels (eg, AKT vs ERK) in T cells
Increased CTL activity/lytic capacity as measured by redirected lysis
Increased IFN-γ/HMGB1 production in TIL/tumor lysate, TIL/autologous tumor and/or TIL/homologous tumor co-cultures.

Additional experiments to be performed include (1) whole exome sequencing and RNASeq on FFPE or fresh-frozen tumor material to identify mutated genes and possible neo-epitopes, (2) cytokine and chemokine analysis of culture supernatants collected 24-48 hours following initiation of tumor fragment cultures, (3) gene expression analysis of tumor fragments removed from early culture on day 7, (4) TCR clonotype analysis of the TIL isolated using high-throughput TCR Vβ CDR3 region sequencing, (5) impact of mAbs on banked TILs for TIL effector function in the presence of IFN-γ induced upregulation of PD-L1 on autologous/homologous tumors (outlined below) and analysis of remaining fragments for residual T-cells by PCR/IHC/digest.

Differences in assay parameters will be tested for significance using paired and un-paired T-tests (Wilcoxon rank-sum and signed rank tests). Comparison of multiple parameters will use one-way and two-way ANOVA analyses. Spearman regression analysis will be used when applicable to assess correlations between continuous measurements. All data can be tabulated and analyzed.

### Example 4—Expansion of TILs Using Hexameric Ligands to 4-1BB, OX40, and Other TNFRSF Members

The effect of activation with hexameric fusion proteins of structures I-A with binding domains to 4-1BB, OX40, CD27, and other TNFRSF members, on expansion and function of tumor infiltrating lymphocytes (TIL) from ex-vivo cultured solid tumor fragments (multiple histologies) is studied in this example. 100 mg of each hexameric fusion protein (e.g., 4-1BB, OX40, and CD27) would be used with tumors obtained from the following indications: sarcoma, colorectal cancer, acute myeloid leukemia, ovarian cancer, triple negative breast cancer, pancreatic (Ras expressing), renal cancer, and bladder cancer. Tumors of various histologies will be obtained from commercial sources. Approximately 20 independent patient tumors will be obtained (2-3 tumors per indication as listed above). Tumors will be shipped to Lion in sterile HBSS or another appropriate medium. The tumors will be handled only in a laminar flow hood to maintain sterile conditions. Alternatively, tumor single cell suspensions may be utilized. The tumors will be washed after receipt and divided into 2-3 mm (length×width×height) fragments and placed into cell culture into 24-well plates (1 fragment per well) or 6-well plates (4 fragments per well) with culture medium supplemented with 6,000 IU/mL IL2 (recombinant) only, combination of 4-1BB HERA alone in triplicates will serve as control and three experimental conditions utilized respectively. An excipient control for the IL-2 will be used. The final concentration of HERA will be 30 μg/mL. Following 24-48 hours of culture, 250 μL of supernatant will be collected from each condition and stored at −20° C. for subsequent analysis of cytokine and chemokine concentrations (pg/10⁶ cell/24 hr). TILs will be collected from each condition on day 11, day 21 and/or day of the ‘pre-REP’ (at least 500,000 cells per sample). Two aliquots of TILs will be pelleted and resuspended in <10 μL of PBS and will be frozen. If less than <10⁶ cells are collected, only gene expression arrays will be performed. Cultures will be fed on day 7 by partial removal of “spent” medium and addition of an equal volume of culture medium plus 6000 IU/mL IL-2. The spent medium will be stored at −20° C. for subsequent cytokine/chemokine analysis using a multiplex assay (e.g., Luminex 100 system). Additional ligand will be added to the culture on day 7 if sufficient tumor fragments are available for initiation of more than 1 replicate of experimental conditions. TIL cultures will be maintained for an additional 14 days.

On day 21, the total cell yield, viability, cell surface and intracellular immunophenotype will be determined using flow cytometry. The following markers will be included: CD45RA, CCR7, CD3, TCR-alpha/beta, CD4, CD8, CXCR3, CD56, CD27, CD28, PD-1, PD-L1, BTLA, KLRG1, CD137, CD134, CD33, CD57, CD25, CD127, TIM-3, LAG-3, TIGIT, RAGE, and Ki67. Other biomarkers including CD107a, NKG2D, KIRS, chemokine death receptors (Fas, DR4) and anti-apoptotic/pro-autophagic proteins (BCL-2, BCL-XL, Bim, CD200, and LC3/HMGB1) will also be assessed if sufficient cells are available. Intracellular markers of cytotoxicity and regulatory T cells, Granzyme B, pSTAT3, pSTAT1, and FOXP3, respectively will be assessed. Lytic potency of TILs will be determined using a lysis assay. In cases where additional tumor material is available and (a) a tumor cell suspension generated following enzymatic digestion, (b) an autologous tumor line generated from aforementioned tumor and/or (c) homologous cell line (if available) will be co-cultured with harvested TIL and IFN-γ release measured. If excess cells are obtained, these will be cryopreserved for isolating RNA and DNA for gene expression analysis by Nanostring Human Immunology Panel (including TCR Vβ clonotyping analysis). If tumor fragments are visible in culture on day 7, they will be harvested and if sufficient cells are available after generation of a single cell suspension, they will be subjected to genetic analysis and flow cytometric phenotypic analysis. Flow cytometric analysis will focus on the phenotype of T cells, dendritic cells, macrophages, B cells, and NK cells after staining using appropriate fluorescent mAb panels. The markers will include: CD11c, CD11b, HLA class II, CD80, CD86, CD83, CD56, CD16, CD19, and CD20.

The criteria used to assess the efficacy of the addition of hexameric fusion proteins to the tumor fragment cultures are summarized above in Example 3, and further optional criteria are described in Table 53.

TABLE 53
Additional criteria for experimental performance for TNFRSF agonist fusion
proteins in TIL expansion processes.

| | | | | | | Increases in |
|---|---|---|---|---|---|---|
| | | | | | | T-cell |
| | | | | | | Central |
| | | | | | ↓Tregs | Memory |
| Threshold of | Cell | | Potency by | | following | (CCR7+, |
| success | Count | Viability | IFNγ | Phenotyping | expansion | CD45RA−) |
| Good | >1e6 | >75% | >500pg/10⁶ | >40% CD8s | <10% | >1% |
| | | | cells/24 hr | | | |
| Very Good | >3e6 | >80% | >1000/10⁶ | >50% CD8s | <3% | >3% |
| | | | cells/24 hr | | | |
| Excellent | >10e6 | >85% | >2000/10⁶ | >75% CD8s | <1% | >10% |
| | | | cells/24 hr | | | |
| Outstanding | >30e6 | >90% | >4000/10⁶ | >90% CD8s | Not detected | >30% |
| | | | cells/24 hr | | | |

Additional experiments include: (1) whole exome sequencing and RNASeq on FFPE or fresh-frozen tumor material to identify mutated genes and possible neo-epitopes, (2) cytokine and chemokine analysis of culture supernatants collected 24-48 hours following initiation of tumor fragment cultures, (3) gene expression analysis of tumor fragments removed from early culture on day 7, (4) TCR clonotype analysis of the TIL isolated using high-throughput TCR Vβ CDR3 region sequencing, (5) impact of hexameric fusion proteins on Lion banked TILs for TIL effector function in the presence of IFN-γ induced upregulation of PD-L1 on autologous/homologous tumors and analysis of remaining fragments for residual T-cells by PCR/IHC/digest.

Differences in assay parameters will be tested for significance using paired and un-paired T-tests (Wilcoxon rank-sum and signed rank tests). Comparison of multiple parameters will use one-way and two-way ANOVA analyses. Spearman regression analysis will be used when applicable to assess correlations between continuous measurements.

### Example 5—Evaluation of the Impact of 4-1BB and Anti-OX40 Agonistic Antibodies on TIL Expansion and Effector Function

The objective of this work is to evaluate the impact of 4-1BB (urelumab) and anti-OX40 agonistic antibodies on TIL expansion and effector function and to obtain information on ICOS and GITR expression during expansion.

In vitro assessment of anti-4-1BB and anti-OX40 agonistic antibodies on TIL expansion and phenotype is performed as follows. Antibody titration is conducted with tumor fragments and aspirates to determine suitable concentration for use with TIL expansion. The impact of anti-4-1BB and anti-OX40 agonists on TIL expansion in both pre-REP and REP (in these specific conditions) is evaluated for (1) IL-2+anti-4-1BB alone, (2) IL-2 anti-OX40 alone, (3) IL-2+anti-41BB+anti-OX40, and (4) IL-2 alone (control). TIL expansion and phenotype will be assessed by (1) expansion of CD3⁺ subset, CD3⁺CD8⁺ subset, and CD3⁺CD4⁺ in both percentage and absolute cell counts and viability, and (2) assessment of differentiation and activation status by flow cytometry using 18 color flow; including staining for ICOS and GITR, Ki67, and apoptosis markers.

In vitro assessment of TCR repertoire and expression profiling of TIL expanded with anti-4-1BB and anti-OX40 agonistic antibodies is performed as follows. TCR repertoire in TILs expanded with IL-2 alone in comparison with treatment conditions is shown by staining with specific anti-TRBV antibodies and using commercially-available TCR repertoire assays from iRepertoire, Inc. Expression profiling on individual TILs is performed using nCounter Vantage™ RNA Adaptive Immunity Panel with Nanostring analysis

In vitro assessment of tumor reactivity and effector function is performed as follows. An autologous tumor cell suspension or tumor cell line is generated (as possible). Tumor reactivity in tumor lysis assay is assessed by co-culturing autologous tumor cells/sorted autologous tumor cell suspension with autologous TIL expanded with IL-2 alone in comparison with treatment conditions described above. In case autologous tumor cell suspensions/tumor cell lines are not available, T cell activation assay by anti-CD3/CD28/CD137 will be conducted to assess effector functions by measuring IFN-gamma production/CD107a expression instead.

### Example 6—Further Evaluation of 4-1BB and OX40 Antibodies on the Ex Vivo Expansion of TIL and their Effector Function Activity

OX40 and 4-1BB have been found to be expressed by antigen specific CD4⁺ and CD8⁺ subset, respectively. Activation of co-stimulatory molecules (4-1BB and OX40) on T-cells enhance effector function, cell survival, and cell expansion. Activation of OX40 and 4-1BB receptors was shown to improve TIL expansion and anti-tumor function in murine models. Anti-4-1BB agonistic antibody was shown to increase the yield of melanoma TIL obtained from in vitro expansion. According to the following protocol, the effect of agonistic antibodies against 4-1BB and OX40, alone and in combination, on the ex vivo expansion of TIL and their effector function activity may be studied.

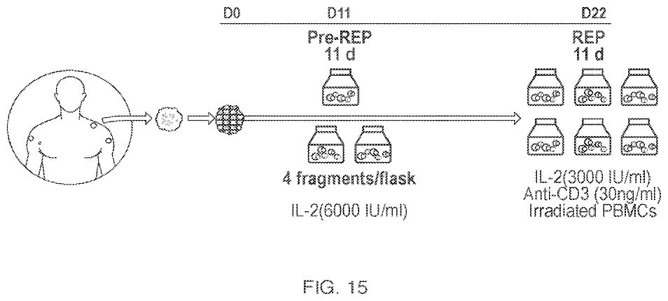

FIG. 13 describes the TIL expansion protocol used in this study. As illustrated in FIG. 15, tumor tissue was retrieved from the patient, fragmented and subjected to a pre-REP process in the presence of IL-2, as described herein. The tissues were then subjected to a REP process in the presence of IL-2 and anti-CD3 anti-bodies with irradiated PBMCs (FIG. 13).

The following experimental conditions were implemented in this study:

| | No | Anti-4- | Anti- | Combi- | |
|---|---|---|---|---|---|
| | treatment | 1BB | OX40 | nation | Isotype |
| pre-REP | | 10 μg/ml | 0.5-10 μg/ml | 0.5-10 μg/ml | 10 μg/ml |
| REP | | 10 μg/ml | 0.5-10 μg/ml | 0.5-10 μg/ml | 10 μg/ml |

| Phase | Agent | Pre-REP | REP |
|---|---|---|---|
| I | anti-4-1BB | 3 tumors | N/A |

| | | |
|---|---|---|
| | anti-OX40 | 3-5 tumors |
| II | combo | 3-5 tumors |

T-Cell activation, proliferation, and exhaustion may be monitored by flow-cytometry according to the following list, where Panel 1 illustrates immune cell lineage, T-cell subsets, and T-cell differentiation, and Panel 2 illustrates T-cell activation and exhaustion:

| Panel 1 | Panel 2 |
|---|---|
| CD3 | CD3 |
| L/D Blue | L/D Blue |
| CD19 | CD25 |
| CD56 | CD95 |
| CD62L | PD-1 |
| CD57 | 2B4/CD244 |
| CD11b | CD4 |
| CD123 | TIM-3 |
| CD14 | CD183 |
| CD8 | CD103 |
| CD28 | CD8 |
| CD45 | TIGIT |
| CD4 | CD127 |
| CCR7 | CD272 |
| CD27 | KLRG1 |
| TCRg/d | CD194 |
| CD45RA | CD69 |
| CD16 | LAG-3 |

Without being limited to any one theory of the invention, it is expected that the combination of anti-4-1BB and anti-OX40 agonists, alone or in combination with process 2A, may improve the expansion of pre-REP TILs, particularly in the CD3⁺CD8⁺ TIL subset; improve the success rate of certain tumors; shorten duration pre-REP TIL expansion; and/or enhance multi-functionalities of TIL including effector function and cell survival following antigen re-stimulation.

### Example 7—Clinical Study to Assess Efficacy and Safety of Autologous TIL

This Example describes a Phase ½ clinical study for evaluating the efficacy of autologous TIL across multiple tumor types. The objectives of this investigation are to evaluate efficacy using objective response rate (ORR) according to RECIST v1.1 in subjects with ovarian cancer and osteosarcoma. The primary objective for a pancreatic ductal adenocarcinoma (PDAC) cohort is to evaluate efficacy as measured by the 6-month survival rate.

Secondary objectives may include: (1) evaluating ORR using RECIST v.1.1 in PDAC; (2) determining the disease control rate (DCR) within and across cohorts; (3) determining the duration of response (DOR); (4) determining progression-free survival (PFS) and overall survival (OS); and (5) further characterizing the safety profile of adoptive cell therapy with TIL across multiple tumor types.

### Definitions/Abbreviations

ACT Adoptive Cell Therapy
AE Adverse Event
ALT Alanine Transaminase
ANC Absolute Neutrophil Count
AST Aspartate Transaminase
ASMR Age-standardized Mortality Rate
aPTT Activated Partial Thromboplastin Time
BID Twice Per Day
BSA Body Surface Area
CBC Complete Blood Count
CD4+T CD4+ T Cells
CD8+T CD8+ T Cells
CFR Code of Federal Regulations
CI Confidence Interval
CLS Capillary Leak Syndrome
CMO Contract Manufacturing Organization
COPD Chronic Obstructive Pulmonary Disease
CR Complete Response
CrCl Creatinine Clearance
CT Computed Tomography
CTCAE v4.03 Common Terminology Criteria for Adverse Events Version 4.03
D5W Dextrose 5% by weight
DCR Disease control rate
DOR Duration of Response
EBV Epstein-Barr Virus
ECHO Echocardiogram
EKG Electrocardiogram
EOC Epithelial Ovarian Cancer
EORTC QLQ-C30 European Organisation for Research and Treatment of Cancer Quality-of-Life Questionnaire—Core 30 instrument
EWV Early Withdrawal Visit
FDA Food and Drug Administration
FEV Forced Expiratory Volume
FVC Forced Vital Capacity
GCP Good Clinical Practice
Hgb Hemoglobin
HIV Human Immunodeficiency Virus
HRQoL Health-related Quality-of-life
ICH International Conference on Harmonization
IL Interleukin
IND Investigational New Drug (Application)
irRECIST Immune-related Response Evaluation Criteria in Solid Tumors
IRB Institutional Review Board
IUD Intrauterine Device
IV Intravenous
IVPB Intravenous Piggyback
LVEF Left Ventricular Ejection Fraction
M1 HLA-DR+CD68+ M1 Macrophages
M2 CD163+ or CD204+ M2 Macrophages
MRI Magnetic Resonance Imaging
MUGA Multiple Gated Acquisition Scan
NCI National Cancer Institute
Neu CD66b+ Neutrophils
NMA Nonmyeloablative
NS Normal Saline
OC Ovarian Cancer
ORR Objective Response Rate
OS Overall Survival
PBMC Peripheral Blood Mononuclear Cell
PCR Polymerase Chain Reaction
PD Progressive Disease
PDAC Pancreatic Ductal Adenocarcinoma
PE Physical Exam
PET Positron Emission Tomography
PFS Progression-free survival
PHI Personal Health Information
PI Principal Investigator
PJP Pneumonitis Jiroveci Pneumonia
PO Per Os (by mouth)
PR Partial Response
PS Performance Status
PT Prothrombin Time
QTc Corrected QT Interval
RECIST Response Evaluation Criteria in Solid Tumors
REP Rapid Expansion Protocol
SAE Serious Adverse Event
SAP Statistical Analysis Plan
SD Stable Disease
SGOT Serum Glutamic-oxaloacetic Transaminase
SGPT Serum Glutamic-pyruvic Transaminase
SMX Sulfamethoxazole
STS Soft Tissue Sarcoma
TCR T-cell Receptor
TIL Tumor-infiltrating lymphocyte
TMA Tissue Microarray
TMP Trimethoprim
Treg FOXP3+ Regulatory T Cells
TSH Thyroid Stimulating Hormone
ULN Upper Limit of Normal

Study Design and Endpoints: This study is aimed at evaluating the efficacy of TIL in subjects with: a) osteosarcomas relapsed or refractory to conventional therapy, b) platinum-resistant ovarian cancer, and c) PDAC who have progressed on, or received maximal benefit from, front-line therapy. Each cohort begins with ten subjects in the first stage, and expansion to the second stage is guided by a modified Simon's two stage design.

The primary endpoint is ORR by RECIST v1.1 for ovarian cancer and osteosarcoma, and the 6-month survival rate in PDAC. The primary endpoint for the PDAC cohort is the 6-month survival rate.

The secondary efficacy endpoints include ORR (for PDAC) CRR, DCR, DOR, PFS using RECIST v1.1, and OS. DCR includes complete response (CR), partial response (PR), and stable disease (SD). Safety endpoints may include overall assessment of AEs including grade 3 or greater non-hematological toxicities, SAEs and treatment-emergent AEs by grade and relationship to the study treatment. The secondary endpoint for the PDAC cohort is ORR using RECIST v1.1.

Exploratory endpoints may include: (1) duration of TIL persistence as determined by T cell receptor (TCR) sequencing of infused T cells serially isolated following TIL infusion, or alternatively iRepertoire assessment of mRNA for the TCRs; (2) response as determined by the immune-related response criteria; (3) immunological Phenotype of TIL at the time of infusion by multichannel flow cytometry; (4) baseline and post-treatment tumor assessment via IHC, TCR sequencing, and transcriptional analysis; and (5) HRQOL as assessed per EORTC QLQ-C30 questionnaire.

Participant Inclusion Criteria. Subjects may be between 18 and 70 (subjects aged 16-70 may be enrolled into the osteosarcoma cohort). Subjects should be willing and able to provide informed consent. For patients<18 years of age, their parents or legal guardians should sign a written informed consent. Assent, when appropriate, may be obtained according to institutional guidelines. Clinical performance status of ECOG 0 or 1 at enrollment and within 7 days of initiating lymphodepleting chemotherapy. Subjects should have an area of tumor amenable to excisional biopsy for the generation of TIL separate from, and in addition to, a target lesion to be used for response assessment. Any prior therapy directed at the malignant tumor, including radiation therapy, chemotherapy, and biologic/targeted agents should be discontinued at least 28 days prior to tumor resection for preparing TIL therapy.

Within 7-14 (e.g., 7 days) days of enrollment and within 12 h to 48 h (e.g., 24 h) of starting lymphodepleting chemotherapy subjects may meet one or more of the following laboratory criteria: (1) absolute neutrophil count (ANC)>1000/mm³; (2) hemoglobin>8.0 g/dL (transfusion allowed); (3) platelet count>100,000/mm³; (4) ALT/SGPT and AST/SGOT<2.5× the upper limit of normal (ULN) (Patients with liver metastases may have LFT≤5.0×ULN); (5) calculated creatinine clearance (Cockcroft-Gault)≥40.0 mL/min; (6) total bilirubin≤1.5×ULN; (7) prothrombin Time (PT) & Activated Partial Thromboplastin Time (aPTT)≤1.5×ULN (correction with vitamin K allowed) unless subject is receiving anticoagulant therapy (which should be managed according to institutional norms prior to and after excisional biopsy); and (8) negative serum pregnancy test (female subjects of childbearing potential).

Furthermore, subjects should not have a confirmed human immunodeficiency virus (HIV) infection. Subjects should have a 12-lead electrocardiogram (EKG) showing no active ischemia and corrected QT interval (QTc) less than 480 ms. Subjects 40 years of age and older should also have a negative stress cardiac test (i.e. EKG stress test, stress thallium, dobutamine echocardiogram or other stress test that may rule out cardiac ischemia). Stress test may be required of subjects less than 40 years of age if warranted by family history or risk factors by the treating investigator. Subjects of childbearing potential should be willing to practice an approved highly effective method of birth control starting at the time of informed consent and for 1 year after the completion of the lymphodepletion regimen. Subjects should be able to adhere to the study visit schedule and other protocol requirements. Finally, pulmonary function tests (spirometry) demonstrating forced expiratory value (FEV) 1 greater than 65% predicted or forced vital capacity (FVC) greater than 65% of predicted.

In addition to meeting the above general inclusion criteria, subjects should also meet cohort specific criteria.

For ovarian cancer, subjects may have high grade non-mucinous histology (carcinosarcomas are allowed). Moreover, subjects may have failed at least two prior lines of chemotherapy (i.e. frontline adjuvant chemotherapy plus one additional line for recurrent/progressive disease).

For osteosarcoma, subjects may have relapsed or become refractory to conventional therapy and have received a regimen including some combination of high-dose methotrexate, doxorubicin, cisplatin, and/or ifosfamide.

For pancreatic adenocarcinoma, subjects may have histologically or cytologically documented diagnosis of PDAC with oligo-metastatic disease. Subjects may have progressed on, or received maximal benefit from, front-line therapy. Patients may have received unlimited lines of prior standard of care therapy. Patients with ascites or carcinomatosis are not eligible for the study. Patients may need an albumin of ≥3.0 mg/dL within 7 days of enrollment.

Participant Exclusion Criteria. A number of criteria may result in exclusion of a participant from the study:
a. Active systemic infections requiring intravenous antibiotics, coagulation disorders or other major medical illnesses of the cardiovascular, respiratory or immune system. PI or his/her designee shall make the final determination regarding appropriateness of enrollment.
b. Patients with active viral hepatitis.
c. Patients who have a left ventricular ejection fraction (LVEF)<45% at Screening.
d. Patients with a history of prior adoptive cell therapies.
e. Persistent prior therapy-related toxicities greater than Grade 2 according to Common Toxicity Criteria for Adverse Events (CTCAE) v4.03, except for peripheral neuropathy, alopecia, or vitiligo prior to enrollment.
f Primary immunodeficiency.
g. History of organ or hematopoietic stem cell transplant.
h. Chronic steroid therapy, however prednisone or its equivalent is allowed at <10 mg/day.
i. Patients who are pregnant or nursing.
j. Presence of a significant psychiatric disease, which in the opinion of the principal investigator or his/her designee, would prevent adequate informed consent.
k. History of clinically significant autoimmune disease including active, known, or suspected autoimmune disease. Subjects with resolved side effects from prior checkpoint inhibitor therapy, vitiligo, psoriasis, type 1 diabetes or resolved childhood asthma/atopy would be an exception to this rule. Subjects that require intermittent use of bronchodilators or local steroid injections would not be excluded. Subjects with hypothyroidism stable on hormone replacement or Sjorgen's syndrome may not be excluded.
l. History of clinically significant chronic obstructive pulmonary disease (COPD), asthma, or other chronic lung disease.
m. History of a second malignancy (diagnosed in the last 5 years). Exceptions include basal cell carcinoma of the skin, squamous cell carcinoma of the skin, or in situ cervical cancer that has undergone potentially curative therapy.
n. History of known active central nervous system metastases and/or carcinomatous meningitis. Subjects with previously treated brain metastases may participate provided they are stable (without evidence of progression by imaging for at least four weeks prior to the first dose of trial treatment and any neurologic symptoms have returned to baseline), have no evidence of new or enlarging brain metastases, and are not using steroids for at least 7 days prior to initiation of lymphodepletion.
o. Has received a live vaccine within 30 days prior to the initiation of lymphodepletion.
p. Any other condition that in the investigator's judgement would significantly increase the risks of participation.

Completion or Discontinuation of Treatment. Completion of treatment may be defined as having received any volume of TIL infusion followed by at least 1 dose of adjuvant IL-2.

This study includes a one-time treatment regimen consisting of lymphodepleting chemotherapy, TIL infusion, and adjuvant IL-2 (up to 6 doses). Discontinuation from study treatment should be considered if any of the following criteria are met. However, unless the patient also meets criteria for discontinuation from study participation, every effort may be made to continue follow-up and assessment of all patients, including those that do not complete the full course of therapy, as specified in the Schedule of Events.

Criteria for early discontinuation from treatment are:
a. Grade 3 or greater autoimmunity that involves vital organs (heart, kidneys, brain, eye, liver, colon, adrenal gland, lungs) with symptoms emerging following TIL infusion;
b. Grade 3 or greater allergic reaction including bronchospasm or generalized urticaria that does not resolve after medical management in the opinion of the Investigator;
c. Grade 3 or greater toxicity due to IL-2 that does not decrease to Grade 2 or less within 96 hours of management;
d. Determination by the Investigator that continued treatment is not in the best interest of the patient;
e. Withdrawal by patient. The patient (or parents/legal guardian for patients<18 years of age) may withdraw consent to treatment but continue consent for follow-up evaluations and/or survival status;
f Pregnancy;
g. Patient meets criteria for early discontinuation from study; and
h. Patient has become ineligible for study after tumor harvest and prior to TIL or IL-2 administration.

Criteria for early discontinuation from study are:
a. Withdrawal by patient. The patient (or parents/legal guardian for patients<18 years of age) may withdraw consent. All efforts should be made to continue consent for survival status follow-up;
b. Patient has become ineligible for study after tumor harvest or did not receive any study treatment;
c. Have any complication or delayed healing from excisional procedure that in the investigator's opinion would increase the risks of lymphodepletion, adoptive TIL therapy and adjuvant IL-2;
d. Have a decline in performance status to ECOG>1 (within seven days prior to starting lymphodepletion);
e. Death; and
f Lost to follow-up after 3 documented attempts to contact the patient.

Some subjects may undergo tumor harvest and TIL manufacture but may not receive the infusion of investigational product. If TIL is not administered to the patient for whatever reason, even if after lymphodepleting chemotherapy, then the patient should remain on study, but data collection may be reduced to survival status and start of any new anticancer therapy for 3 years. Such subjects may be considered unevaluable for statistical analysis of efficacy and may be replaced.

If a patient initiates anti-cancer therapy or exhibits disease progression after TIL infusion they may remain in the study, but the data collection may be reduced to response status, survival status and other anti-cancer therapy for 3 years.

Study Agents. The lymphodepletion regimen is scheduled to start on Day −7, after notification that TIL production is expected to be successful for the patient. Patients may receive lymphodepleting chemotherapy as inpatient or outpatient at the discretion of the investigator. Modification of the lymphodepletion regimen is allowed as clinically indicated and should be guided by daily hematological parameters as described below for fludarabine in heavily pre-treated patients or subjects with a history of prolonged myeloid recovery. The regimen comprises 2 daily doses of cyclophosphamide (with mesna) followed by 5 daily doses of fludarabine and should be administered as per institutional protocol/standards for nonmyeloablative chemotherapy. Guidelines for preparation and administration are described below. Subjects should be dosed using actual body weight but not to exceed 140% of Ideal Body Weight as defined below:
a. Ideal Body Weight for Males=50 kg+2.3×(number of inches over 60 inches in height).
   Example: ideal body weight of a 5′10″ male subject 50+2.3×10=73 kg
b. Ideal Body Weight for Females=45.5 kg+2.3 (number of inches over 60 inches in height).
   Example: ideal body weight of a 5′3″ female subject 45.5+2.3×3=52.4 kg

Drugs required for lymphodepletion include cyclophosphamide, fludarabine, and/or mesna.

Variations from the lymphodepletion (e.g. infusion times; schedule of treatments, etc.) prior to day 0 may be documented in the medical record but may not be considered protocol violations/deviations.

Cyclophosphamide may be administered at 20 to 80 mg/kg/day (e.g., 60 mg/kg/day) IV in 250 mL normal saline (NS) over approximately 2 hours on Days −7 and −6. Mesna 60 mg/kg with dextrose 5% by weight (D5W) or NS infused intravenously over 24 h on Days −7 and −6. As noted above the dose may be based on the patient's actual body weight, but to prevent undue toxicity, it may not exceed the dose based on 140% of the maximum ideal body weight (defined above). There may be dose adjustments for cyclophosphamide.

Fludarabine will then be infused at 15 to 50 mg/m² (e.g., 25 mg/m²) IV piggyback (PB) daily over approximately 15-30 minutes on Days −5 to −1. To prevent undue toxicity with fludarabine, the dose may be based on body surface area (BSA), but may not exceed a dose calculated on surface areas based on body weights greater than 140% of the maximum ideal body weight. Hematological parameters (complete blood count [CBC] and differential) are to be reviewed daily during lymphodepletion. If after 3 or 4 doses of fludarabine, the absolute lymphocyte count falls below 100 cells/mm³ the remaining dose(s) of fludarabine may be omitted following discussion with the PI. Fludarabine dose may be adjusted according to estimated creatinine clearance (CrCl) as follows: (1) CrCl 50-79 mL/min: Reduce dose to 20 mg/m²; and/or (2) CrCl 40-49 mL/min: Reduce dose to 15 mg/m².

The TIL product that may be used in this protocol is a cellular investigational product comprising a live cell suspension of autologous TIL derived from the patient's own tumor. Each dose may contain up to 150×10⁹ total viable lymphocytes. The total volume to be infused may be up to 600 mL dependent on total cell dose.

If not already hospitalized for the lymphodepleting chemotherapy, the patient may be admitted 1-2 days prior to planned TIL administration and prepared with overnight intravenous hydration prior to the TIL, administration. Patients may remain hospitalized until the completion of the IL-2 therapy, as per institutional standards.

The IL-2 infusion may begin 3-24 h after completion of the TIL infusion. IL-2 may be administered at a dose of 200,000 to 1,000,000 IU/kg (e.g., 600,000 IU/kg) (based on total body weight) and may be administered by IV infusion at a frequency of every 8-12 hours as per institutional standard of care and continued for up to a maximum of six doses or as tolerated. IL-2 doses may be skipped if patient experiences a Grade 3 or 4 toxicity due to IL-2 except for reversible Grade 3 toxicities common to IL-2 such as diarrhea, nausea, vomiting, hypotension, skin changes, anorexia, mucositis, dysphagia, or constitutional symptoms and laboratory changes. Management of IL-2 is detailed in Table 54. If these toxicities can be easily reversed within 24 hours by supportive measures, then additional doses may be given. If greater than 2 doses of TIL-2 are skipped, IL-2 administration may be stopped. In addition, discretion may be used to hold or stop the dosing.

TABLE 54
Management of Potential Aldesleukin Toxicities.

| Expected | Expected | | |
|---|---|---|---|
| toxicity | grade | Supportive Measures | Stop Treatment* |
| Chills | 3 | IV Meperidine 25-50 | No |
| | | mg, IV q1h, prn | |
| Fever | 3 | Acetaminophen 650 | No |
| | | mg, | |
| | | po, q4h; Indomethicin | |
| | | 50-75 mg, po, q8h | |
| Pruritis | 3 | Hydroxyzine HCL | No |
| | | 10-20 | |
| | | mg po q6h, prn; | |
| | | Diphenhydramine | |
| | | HCL25-50 mg, po, q4h, | |
| | | prn | |
| Nausea/ | 3 | Ondansetron 10 mg, | No |
| Vomiting/ | | IV, | |
| Anorexia | | q8h, prn; Granisetron | |
| | | 0.01 | |
| | | mg/kg IV daily prn; | |
| | | Droperidol 1 mg, IV q4- | |
| | | 6h, prn; Prochlorperazine | |
| | | 25 mg q4h p.r., prn or 10 | |
| | | mg IV q6h prn | |
| Diarrhea | 3 | Loperamide 2 mg, po, | If uncontrolled after |
| | | q3h, prn; Diphenoxylate | 24 hours despite |
| | | HCl 2.5 mg and atropine | all supportive |
| | | sulfate 25 mcg, po, q3h, | measures |
| | | prn; codeine sulfate | |
| | | 30-60 | |
| | | mg, po, q4h, prn | |
| Malaise | 3 or 4 | Bedrest interspersed | If other toxicities |
| | | with activity | occur |
| | | | simultaneously |
| Hyperbili- | 3 or 4 | Observation | If other toxicities |
| rubinemia | | | occur |
| | | | simultaneously |
| Anemia | 3 or 4 | Transfusion with | If uncontrolled |
| | | PRBCs | despite all |
| | | | supportive measures |
| Thrombo- | 3 or 4 | Transfusion with | If uncontrolled |
| cytopenia | | platelets | despite all |
| | | | supportive measures |
| Neutropenia | 4 | Observation | No |
| Edema/ | 3 | Diuretics prn | No |
| Weight gain | | | |
| Hypotension | 3 | Fluid resuscitation; | If uncontrolled |
| | | Vasopressor support | despite all |
| | | | supportive measures |
| Dyspnea | 3 or 4 | Oxygen or ventilatory | If requires |
| | | support | ventilatory |
| | | | support |
| Oliguria | 3 or 4 | Fluid boluses or | If uncontrolled |
| | | dopamine at renal doses | despite all |
| | | | supportive measures |
| Increased | 3 or 4 | Observation | Yes (grade 4) |
| creatinine | | | |
| Renal | 3 or 4 | Dialysis | Yes |
| failure | | | |
| Pleural | 3 | Thoracentesis | If uncontrolled |
| effusion | | | despite all |
| | | | supportive measures |
| Bowel | 3 | Surgical intervention | Yes |
| perforation | | | |
| Confusion | 3 | Observation | Yes |
| Somnolence | 3 or 4 | Intubation for airway | Yes |
| | | protection | |
| Arrhythmia | 3 | Correction of fluid and | If uncontrolled |
| | | electrolyte imbalances; | despite all |
| | | chemical conversion or | supportive measures |
| | | electrical conversion | |
| | | therapy | |
| Elevated | 3 or 4 | Observation | Yes |
| Troponin | | | |
| levels | | | |
| Myocardial | 4 | Supportive care | Yes |
| Infarction | | | |
| Elevated | 3 or 4 | Observation | For grade 4 without |
| trans- | | | liver |
| aminases | | | metastases |
| Electrolyte | 3 or 4 | Electrolyte | If uncontrolled |
| imbalances | | replacement | despite all |
| | | | supportive measures |

*Unless the toxicity is not reversed within 12 hours.

Study Procedures and Schedule. The following procedures may be used in this study.

Potential subjects may be informed about the study by the investigator. The risks, benefits, and alternatives may be discussed and the Informed Consent Document may be signed before any study related assessments are performed.

Subjects should meet most, or preferably all, inclusion criteria and preferably do not have any of the conditions specified in the exclusion criteria. Confirmation of general, cohort, specific, and treatment inclusion/exclusion criteria should be documented within seven days of starting lymphodepletion chemotherapy.

The demographic data may include date of birth (as allowed per local regulations), age, gender, and race/ethnic origin.

Relevant and significant medical/surgical history and concurrent illnesses may be collected for all patients at Screening (Visit 1) and updated as applicable. Any worsening from pre-existing conditions should be reported as AEs. Patient's prior anti-cancer treatment may also be collected.

Documentation of cohort-specific diagnosis of cancer may be made and confirmed histologically.

All medications and therapies (prescription, and non-prescription, including herbal supplements) taken by the patient up to 28 days prior to Screening (Visit 1) may be collected in the database, including the stop dates for medications prohibited in the study, at the time of consent. All medications and therapies being taken by the patients, or changes thereof, at any time during the study, may be recorded in the medical record.

All baseline grade 2 and higher toxicities may be assessed as per CTCAE v4.03. Any events occurred after screening, but prior to enrollment/tumor resection, may be recorded as Medical History in the database, unless the events are related to protocol mandated procedures. Any events occurring after enrollment/tumor resection may be captured as AEs in the database until the 6 Month visit, subject is taken off the study, or starts other cancer therapy.

Vital signs shall include height, weight, pulse, respirations, blood pressure and temperature. Height may be measured at Screening (Visit 1) only. All other vital signs may be measured at applicable time points. On Day 0 (Visit 11/TIL infusion), vital signs may be monitored for up to approximately 24 hours post TIL infusion.

An ECOG performance status may be assessed at Screening (Visit 1) and other time points indicated on the schedule of events.

Physical examination may be conducted for all visits except for Tumor Resection and shall include vital signs and weight, head and neck, cardiovascular, pulmonary, extremities, and other relevant evaluation. Exams during conducted during follow-up may be symptom directed. Clinically significant changes in the exam findings may be recorded as adverse events as indicated.

Safety blood and urine tests may be collected and analyzed locally at every visit as indicated in the Schedule of Events.

Sample collection for high resolution HLA Class I typing may be conducted at Screening (Visit 1).

Serology for the following diseases may be completed at Screening (Visit 1) to be analyzed locally per institutional standard: HIV, Hepatitis B Virus, Hepatitis C Virus, Cytomegalovirus (CMV), Herpes Simplex Virus; Epstein-Barr virus (EBV) (may be within previous 3 months to Tumor Resection/Visit 2), Chagas Disease, Human T cell Lymphotropic Virus, and West Nile Virus. Sickle Cell Disease may also be screened. Additional testing is to be done as clinically indicated.

The creatinine clearance may be calculated by site using the Cockcroft-Gault formula at Screening only.

All subjects can have a baseline 12-lead ECG and assessment of ventricular function by echocardiogram or MUGA. In addition, subjects age 40 or older and those younger than 40 with a history of cardiovascular disease or chest pain may have a stress test documenting absence of ischemia. Patients with an abnormal MUGA or echocardiogram may meet ejection fraction requirements and obtain cardiology clearance prior to enrollment.

Pulmonary evaluation may be completed within 28 days from Screening (Visit 1). Prior evaluations completed within 6 months prior to Screening (Visit 1) may be accepted. An FEV1 greater than 65% of predicted or FVC greater than 65% of predicted is required. Patients who are unable to conduct reliable PFT spirometry measurements due to abnormal upper airway anatomy (e.g. tracheostomy) may undergo a 6-minute walk test to be evaluate pulmonary function. These patients should, and preferably can, walk a distance of at least 80% predicted for age and sex as well as maintain oxygen saturation greater than 90% throughout.

Colonoscopy is only required for patients who have had a documented Grade 2 or greater diarrhea or colitis due to previous immunotherapy within six months of Screening. Patients that have been asymptomatic for at least 6 months from Screening or had a normal colonoscopy post anti-PD-1/anti-PD-L1 treatment, with uninflamed mucosa by visual assessment may not need to repeat the colonoscopy.

A health related quality of life (HRQOL) questionnaire may be conducted in person at baseline Day −21 (Visit 3) and be performed as the first procedure on the subsequent visits. See the Schedules of Events for specific timepoints. Failure to complete any questionnaires may not be considered a deviation requiring reporting.

Radiographic assessments by computed tomography (CT) scans with contrast of the chest, abdomen and pelvis are required for all patients for tumor assessments. CT scans are performed as indicated in the Schedule of Events until progressive disease by modified RECIST v1.1 is noted (or if the patient withdraws full consent). Response assessments should be evaluated and documented by a qualified radiologist participating in the trial. Magnetic Resonance Imaging (MRI) or positron emission tomography (PET) scans of the chest, abdomen and pelvis in lieu of CT scans may be allowed for patients who have an intolerability to contrast media. The same method of assessment (CT or MRI) and the same technique for acquisition of data should be used consistently throughout the study to characterize each identified and reported lesion. Initial radiographic assessments may be made at 6, 12, 18 and 24 weeks post TIL infusion. Thereafter, Patients may be evaluated for response approximately every 12 weeks. Additional radiological assessments may be performed as clinically indicated.

Prior to surgical biopsy, subject eligibility may be confirmed, and the PI or designee may provide approval for patient enrollment into the clinical trial and subsequent tumor resection. Subjects may undergo a pre-procedural consultation and a separate informed consent by the team performing the surgical biopsy per institutional standards. Ideally, the targeted tumor should have not been previously irradiated. If the tumor has been previously irradiated a minimum period of 1 to 6 months (e.g., 3 months) may have elapsed between irradiation and resection, during which time additional target-tumor growth may have been demonstrated. If enrolled, tumor resection is expected to occur approximately 1 to 12 weeks (e.g., 6 weeks) prior to the anticipated TIL infusion (Day 0). TIL is an autologous investigational product which is procured and delivered by means that have more in common with autologous blood product delivery than those of traditional drug production. It is imperative that only the patient's own (autologous) study treatment (TIL) be administered to the same individual patient. For these reasons, the patient specimen can be procured and handled per a strict protocol to ensure optimal quality of the specimen and minimum transport time to and from the processing lab facility, as well as to ensure the appropriate identification of the study product at all times including infusion back into the patient.

In cases where additional or excess tumor tissue can be safely procured at the time of the initial excisional biopsy for TIL harvest, excess tumor tissue for research may be procured. Provision of adequate amount of tumor tissue for TIL manufacturing is priority over the collection of additional tumor tissue that is sent for research. Every effort should be made to obtain adequate tumor tissue for both TIL manufacturing and additional analysis. In addition, a mandatory on-study biopsy may be used to ascertain molecular and immunological changes following treatment and as well as to document presence of infused T cells in the tumor. The tumor tissue analysis may include: 1) immunohistochemistry to identify individual immune cell populations; and/or 2) DNA and RNA analysis, including possible exploratory genomic and transcriptomic evaluation and TCR sequencing to evaluate infused TIL homing to tumor (in the post-treatment biopsy). Provision of adequate amount of tumor tissue for TIL manufacturing is priority over the collection of additional tumor tissue for research. Every effort should be made to obtain adequate tumor tissue for both TIL manufacturing and additional analysis.

Up to 500×10⁶ TIL from the infusion product (and genetic material extracted from these samples) may be stored for research. Flow cytometry analysis of the infused TIL may be performed, and DNA from the infusion product may be sent for TCR sequencing. The samples in these research studies may be used to gain further information about the disease and the characteristics of the TIL before and after infusion. Peripheral blood may be collected from the patients for immune monitoring and T cell tracking using TCR sequencing. Blood for Immune Monitoring may be drawn at Tumor Resection (Visit 2) and subsequent collections may be drawn at applicable time points (See Tables 55 and 56).

TABLE 55
Exemplary Schedule of Events-Pre-Treatment Treatment Phases.

| | | | | Treatment Phase | | | | | | | | |
|---|---|---|---|---|---|---|---|---|---|---|---|---|
| | Pre-treatment Phase | | | | | 6, 7, | | 12, 13, | | | | |
| Visit Number | 1 | 2 | 3 | 4 | 5 | 8, 9, 10 | 11 | 14, 15 | 16 | 17 | 18 | 19 |
| | | | Baseline | | | Days −5, | Day 0 | Days | | | Day | Day |
| | | | (Day −14 | | | −4, −3, | (TIL | 1, 2 | Day | Day | 42 | 84 |
| Visit Name | Screening | Biopsy | to −21) | Day −7 | Day −6 | −2, −1 | Infus.) | 3, 4 | 14 | 28 | (Wk 6) | (Wk 12) |
| Visit Window | ≤28 days | N/A | N/A | N/A | N/A | N/A | N/A | N/A | (±7 | (±7 | (±7 | (±7 |
| | | | | | | | | | days) | days) | days) | days) |
| Written | X | | | | | | | | | | | |
| Informed | | | | | | | | | | | | |
| Consent | | | | | | | | | | | | |
| Medical | X | | | | | | | | | | | |
| History | | | | | | | | | | | | |
| Documentation | X | | | | | | | | | | | |
| of diagnosis | | | | | | | | | | | | |
| Physical Exam | X | | X | X | X | X | X | X | X | X | X | X |
| Vital Signs^{a} | X | X | X | X | X | X^{b} | X | X | X | X | X | X |
| ECOG | X | X | X | X | | | X | | | X | X | X |
| performance | | | | | | | | | | | | |
| status | | | | | | | | | | | | |
| CBC, Chem | X | X | X | X | X | X | X | X | X | X | X | X |
| Panel, and | | | | | | | | | | | | |
| urine tests^{c} | | | | | | | | | | | | |
| β-HCG Serum | X | | X | X | | | | | | | | |
| Pregnancy | | | | | | | | | | | | |
| Test | | | | | | | | | | | | |
| Infection | X | | | | | | | | | | | |
| testing | | | | | | | | | | | | |
| HLA typing | X | | | | | | | | | | | |
| Cardiac | X | | | | | | | | | | | |
| Evaluations | | | | | | | | | | | | |
| Pulmonary | X | | | | | | | | | | | |
| function tests | | | | | | | | | | | | |
| Colonoscopy | X | | | | | | | | | | | |
| Tumor | X | | X | | | | | | | | X | X |
| Assessments | | | | | | | | | | | | |
| (CT/MRI) | | | | | | | | | | | | |
| Response | | | | | | | | | | X | X | X |
| Assessments | | | | | | | | | | | | |
| Concomitant | X | X | X | X | X | X | X | X | X | X | X | X |
| Meds | | | | | | | | | | | | |
| Adverse | X | X | X | X | X | X | X | X | X | X | X | X |
| events | | | | | | | | | | | | |
| Tumor Biopsy | | X | | | | | | | | | X | |
| NMA | | | | X | X | X | | | | | | |
| lymphodepletion^{d} | | | | | | | | | | | | |
| TIL Infusion^{e} | | | | | | | X | | | | | |
| IL-2^{f} | | | | | | | | X | | | | |
| Immune | | X | | X | | | | | X | X | X | X |
| Monitoring | | | | | | | | | | | | |
| HRQOL | | | X | | | | | | | | | X |
| Questionnaire | | | | | | | | | | | | |

Prophylactic Medications

| | | | | | | | | | |
|---|---|---|---|---|---|---|---|---|---|
| PJP | X | X | X | X | X | X | X | X | X |
| Filgrastim | | | | | X | | | | |
| Fungal | | | | X | X | X | X | X | X |
| Prophylaxis | | | | | | | | | |
| Herpes Virus | | | | X | X | X | X | X | X |
| Prophylaxis | | | | | | | | | |

^{a}Vital signs may include height, weight, heart rate, respiratory rate, blood pressure, and temperature. Height may be measured at Screening only. BSA and BMI may be Calculated at Day −7 (Visit 4) only.
^{b}On Day 0 (TIL infusion), vital signs may be monitored every 30 minutes during infusion then hourly (+/−15 minutes) for four hours and then routinely (every four to six hours), unless otherwise clinically indicated, for up to approximately 24 hours post TIL infusion.
^{c}Chemistry: sodium, potassium, chloride, total CO2, or bicarbonate, creatinine, glucose, BUN, albumin, calcium, magnesium, phosphorus, alkaline phosphatase, ALT/SGPT, AST/SGOT, total bilirubin, direct bilirubin, LDH, total protein, total CK, uric acid, and serum creatinine. Thyroid panel (to include TSH and Free T4) is to be done at Visits 1 and 19 and as clinically indicated. Coagulations: PT, PTT, and INR. Hematology: CBC with differential; Urinalysis: Bilirubin, Blood, Glucose, Ketones, pH, Protein, Specific gravity, Color and Appearance.
^{d}Cyclophosphamide with mesna for 2 days at Day −7 and Day −6 (Visits 4 thru 5) followed by 5 days of fludarabine at Day −5 thru Day −1 (Visits 6 thru 10).
^{e}TIL infusion is to be done 1 to 2 days after the last dose of agent in the NMA lymphodepletion regimen.
^{f}Initiate IL-2 at 600,000 IU/kg within approximately 3 to 24 hours after TIL infusion and continue every 8-12 hours for up to six doses.

TABLE 56
Exemplary Schedule of Events: Post-Treatment and Long-Term Follow-Up.

| | Post-treatment Follow-up | | | | | | | Long-term |
|---|---|---|---|---|---|---|---|---|
| Visit Number | 20 | 21 | 22 | 23 | 24 | 25 | EWV | Follow-up |
| | Day 126 | Day | Day | Day 336 | Day 504 | Day 672 | Early | |
| | (Month 4.5/ | 168 | 252 | (Month | (Month | (Month | Withdrawal | Quarterly |
| Visit Name | Week 18) | (Month 6) | (Month 9) | 12) | 18) | 24) | Visit | Contact |
| Visit Window | (±14 | (±14 | (±14 | (±14 | (±21 | (±21 | | (±21 |
| | days) | days) | days) | days) | days) | days) | | days) |
| Physical Exam^{a} | X | X | X | X | X | X | X | |
| Vital Signs^{b} | X | X | X | X | | | X | |
| ECOG performance status | X | X | X | X | X | X | X | |
| CBC, Chem Panel, and | X | X | | | | | X | |
| urine tests^{c} | | | | | | | | |
| Tumor Assessments | X | X | X | X | X | X | X | |
| (CT/MRI)^{d} | | | | | | | | |
| Response Assessments | X | X | X | X | X | X | X | |
| Concomitant Meds | X | X | X | X | | | X | |
| Adverse events^{e} | X | X | | | | | X | |
| Immune Monitoring^{f} | | X | X | X | | | X | |
| HRQOL Questionnaire | | X | | X | | X | | |
| Survival Follow-up | | | | | | | | X |

Prophylactic Medications

| | | |
|---|---|---|
| PJP | X | X |
| Herpes Virus Prophylaxis | X | X |

^{a}PE may include gastrointestinal (abdomen, liver), cardiovascular, extremities, head, eyes, ears, nose, and throat, respiratory system, skin, psychiatric (mental status), general nutrition. PE conducted during follow-up may be symptom directed.
^{b}Vital signs may include weight, heart rate, respiratory rate, blood pressure, and temperature.
^{c}Chemistry: sodium, potassium, chloride, total CO2, or bicarbonate, creatinine, glucose, BUN, albumin, calcium, magnesium, phosphorus, alkaline phosphatase, ALT/SGPT, AST/SGOT, total bilirubin, direct bilirubin, LDH, total protein, total CK, uric acid, and serum creatinine. Thyroid panel (to include TSH and Free T4) is to be done as clinically indicated. Coagulations: PT/PTT/INR. Hematology: CBC with differential; Urinalysis: Bilirubin, Blood, Glucose, Ketones, pH, Protein, Specific gravity, Color and Appearance.
^{d}CT Scans of the chest, abdomen and pelvis, are required at the indicated time points. Additional radiological assessments may be performed per Investigator's discretion. MRI may be used if patients are intolerable to contrast media.
^{e}Any AEs occurred after Screening, but prior to enrollment/tumor resection, may be recorded as Medical History in the database. Any AEs occurred after enrollment/tumor resection may be captured as AEs through Day 168 (Visit 21/Month 6) and as clinically indicated, or until the first dose of the subsequent anti-cancer therapy, whichever occurs first. All AEs attributed to protocol-required procedures or treatment may be collected through Day 672 (Visit 25/Month 24).
^{f}Blood draw for Immune Monitoring is to be collected at visits between Day 168 (Visit 21/Month 6) through Day 336 (Visit 23/Month 12) and ETV.

Concomitant Medications, Treatments, and Procedures. Medications for medical problems other than antineoplastic agents are permitted. Those with conditions requiring anti-inflammatory drugs for chronic conditions potentially affecting TIL administration may be considered only with approval of the PI. Palliative radiation therapy is permitted between tumor resection and lymphodepletion as long as it does not affect target and non-target lesions. Use of systemic steroid therapy≤10 mg/day of prednisone or equivalent is permitted. Use of >10 mg/day of prednisone or equivalent is permitted in cases of exacerbation of known disease or for treatment of new symptoms on study per Investigator's discretion. Any changes in concomitant medications may be recorded only in the patient's medical record throughout the trial. For subject who have CT IV contrast allergy, radiologic evaluation using MRI or PET-CT (without intravenous contrast is the preferred management. Every attempt should be made to maintain consistency in imaging modality for each patient.

All other anti-neoplastic drugs and radiation are prohibited. Subjects are also discouraged from using over-the-counter supplements and homeopathic products, especially those with purported anti-inflammatory properties, such as boswelia.

Patients treated with lymphodepletion are subject to opportunistic infections and appropriate infectious agent prophylaxis is required. The prophylaxis regimens and duration listed below may be modified as clinically indicated in consultation with an Infectious Diseases specialist.

Patients may receive levofloxacin at 100 to 1000 mg (e.g., 500 mg) daily (or an equivalent antibiotic) until ANC recovers to greater than 500/mm³.

Patients may receive the fixed combination of trimethoprim (TMP) and sulfamethoxazole (SMX) as double strength (DS) tablet [DS tabs=TMP 160 mg/tab and SMX 800 mg/tab] PO BID twice a week. TMP/SMX-DS may be taken by patients beginning on Day −7 and continuing for a minimum of 6 months after lymphodepletion. For patients with sulfa allergies, Pentamidine may be given (once discharged from the hospital) 300 mg IV every 21 days for 6 months after lymphodepletion. If IV Pentamidine is not feasible after discharge, PCP prophylaxis can be substituted with oral antimicrobials such as Atovaquone as per standard of care for 6 months after lymphodepletion. Patients may be given prophylactic antibiotics intravenously during high dose IL-2 therapy.

Starting on the day of TIL infusion subjects may be administered valacylcovir 100 to 1000 mg (e.g., 500 mg) PO daily if patient is able to take oral medications or acyclovir 5 mg/kg IVPB every 8 hours if patient needs intravenous medications, which is continued for 6 months (or at the discretion of the treating physician). Reversible renal insufficiency has been reported with IV administered acyclovir but not with oral acyclovir. Neurologic toxicity including delirium, tremors, coma, acute psychiatric disturbances, and abnormal electroencephalograms has been reported with higher doses of acyclovir. If symptoms occur, a dosage adjustment may be made or the drug be discontinued. Acyclovir may not be used concomitantly with other nucleoside analogs (e.g. ganciclovir), which interfere with DNA synthesis. In patients with renal disease, the dose is adjusted as per product labeling.

Patients may begin Fluconazole 50 to 500 mg (e.g., 200 mg) PO daily with the T cell infusion (Day 0) and continue for 6 months (or at the discretion of the treating physician).

To reduce the duration of neutropenia following NMA lymphodepletion chemotherapy, filgrastim (G-CSF) may be given at 1 to 10 μg/kg/day (e.g., 5 μg/kg/day) daily subcutaneously until ANC>500/mm³ for at least 2 consecutive days. Approximate dosing to correspond to the 300 mcg or 480 mcg dosage forms is allowed.

Ondansetron may be used to control nausea and vomiting during the chemotherapy preparative regimen. It can cause headache, dizziness, myalgias, drowsiness, malaise, and weakness. Less common side effects include chest pain, hypotension, pruritus, constipation and urinary retention. Consult the package insert for a complete list of side effects and specific dose instructions.

Furosemide may be used to enhance urine output during the chemotherapy preparative regimen with cyclophosphamide. Adverse effects include dizziness, vertigo, paresthesias, weakness, orthostatic hypotension, photosensitivity, rash and pruritus. Consult the package insert for a complete list of side effects and specific dose instructions.

Patients may start on broad-spectrum antibiotics, either a 3^{rd} or 4^{th} generation cephalosporin with adequate pseudomonas coverage as per local antibiogram or a quinolone for temperature≥38.5° C. with an ANC less than 500/mm³. Aminoglycosides should be avoided if possible. Infectious disease consultation may be obtained from all patients with unexplained fever or any infectious complications.

Using daily CBC values as a guide, the patient may also receive platelets and packed red blood cells as needed. Attempts may be made to keep Hgb>8.0 g/dL, and platelets>20,000/mL guided by the clinical scenario. Leukocyte filters may be utilized for all blood and platelet transfusions to decrease sensitization to transfused WBC's and decrease the risk of CMV infection. Irradiated blood and blood products should be used.

Description of Statistical Methods. The primary endpoint for ovarian cancer and osteosarcoma cohorts is the ORR as assessed by investigators using RECIST 1.1 criteria. The ORR is derived as the sum of the number of patients with a confirmed CR or partial response (PR) divided by the number of patients in the All-Treated analysis set×100%. The primary endpoint for the cohort of PDAC is the percentage of patients who survive for 183 days. The 6-month landmark survival rate may be calculated based on the Kaplan Meier method.

PFS is defined as the time (in months) from the start date of lymphodepletion to PD or death due to any cause, whichever event is earlier. Patients not experiencing PD or death at the time of data cut or end of study (i.e., database lock) may have their event times censored on the last adequate tumor assessment. DOR is measured from the first time measurement criteria are met for a CR or PR, whichever response is observed first, until the first date that progressive disease (PD) or death occurs. Patients not experiencing PD or death prior to the time of data cut or end of study may have their event times censored on the last adequate tumor assessment. The analysis of DOR is based on responders only as assessed by investigators per RECIST v1.1. DCR is derived as the sum of the number of patients who achieved PR/CR or SD per the RECIST v1.1 divided by the number of patients in the All-Treated analysis set×100%. OS is defined as the time (in months) from the start date of the lymphodepletion to death due to any cause. Patients not having expired at the time of data cut or end of study may have their event times censored on the last date of their known survival status.

All exploratory analyses may be descriptive and performed by cohort. Some analysis results may be reported separated from the final clinical study report. T-cell repertoire analysis may be used to determine TIL persistence. Molecular and immunological features of tumors before and after TIL therapy may be determined using exome sequencing and immunohistochemistry/immunofluorescence analyses. Sensitivity analyses on ORR, DCR, DOR, and PFS as measured by investigators using the irRECIST criteria may be performed. Pearson correlation coefficient and linear regression, when appropriate, may be used to quantify the relationship between phenotypic attributes (CD8%, CD27 and CD28 expression, etc.) and tumor response to therapy. Baseline CA19-9 of patients with PDAC and baseline CA-125 of patients with ovarian cancer may be assessed for potential correlations with the efficacy outcome.

Grade 3 or higher treatment-emergent AEs and their incidence rates may be compared descriptively to historical data of TIL in other cancer disease types. AE incidence rates may be estimated with 95% CIs per cohort and all cohorts combined. The treatment-emergent AEs start from the first dose of cyclophosphamide and up to 6 months from the last dose of IL-2.

A study disposition summary may display number and percentages of patients who exit the study early by the primary reason in 2 parts: (1) After the tumor harvest prior to lymphodepletion; and (2) On or after the first dose of cyclophosphamide. Patients who are treated and being followed for the survival status at the time of study termination (i.e., completers) are not a part of this summary. Patients who did not receive planned full study treatment doses may also be summarized by its primary reason.

Summary of tumor response data per cohort may be based on the best overall response as assessed by investigators per RECIST 1.1. The summary may display percentages with 80% confidence intervals (CIs) for ORR and 95% CIs for DCR by the Wilcoxon score method among patients in the All-Treated analysis set. The median time-to-event and the landmark rate may also be measured with 80% CIs for the 6-month survival rate and 95% CIs for DOR, PFS, OS, and other landmark rates by the KM method.

All exploratory analyses may be descriptive and performed by cohort. The analysis may be defined separately from the statistical analysis plan for this study and reported independently outside the clinical study report (CSR). HRQOL may be assessed using the EORTC QLQ-C30 instrument and analyzed per the published evaluation manual.

Sample Size. For ovarian cancer and osteosarcoma, the Simon's two stage minimax design may be used to monitor the efficacy of each cohort independently. The null hypothesis that the historical response rate of 5% to be tested against the estimated experimental cohort response rate of 20%. In the first stage, 10 patients may be treated per cohort. If there is no confirmed response in these 10 patients, so long as the patient are evaluable, the cohort may be terminated. Other efficacy estimates including maximum % reductions in target lesion sum of diameters and/or time to PD/death may be considered for termination. A confirmed response shall be determined by RECIST 1,1 criteria with first assessment at 6 weeks and second confirmatory scan at 12 weeks. If the study moves forward to Stage II, an additional 8 patients may be treated leading to a total of 18 patients for that cohort. Three or more responders out of 18 treated patients for the cohort may be considered clinically relevant to justify further investigation. The power of this design is >=70% under the 1-sided type I error rate of 10%.

For PDAC, the Simon's minimax two-stage design may also be used to monitor the 6-month survival rate. The null hypothesis that the historical 6-month survival rate of 35% to be tested against the estimated experimental cohort survival rate of 5000 (ASCO January 2016). In the first stage, 11 patients may be treated and followed for ≥6 months without holding further enrollment. If there are 8 or more deaths among first 11 patients within 183 days counting from the first study drug administration, this cohort may be considered termination.

Otherwise, an additional 11 patients may be treated for a total of 22. The final result for the cohort may be clinically meaningful if 10 patients survive at least for 183 days. The power of this design is approximately 700% under the 1-sided type I error rate of 10%.

### Example 8—Methods of Expanding TILs Using TNFRSF Agonists During Pre-REP and REP Steps

The antibodies used in this Example are described elsewhere herein and are further described in Table 57.

TABLE 57
4-1BB and OX40 Agonistic Monoclonal Antibodies.

| | 4-1BB | | OX40 |
|---|---|---|---|
| Short name | CB 4-1BB | BPS 4-1BB | CB OX40 |
| Source | Creative Biolabs | BPS Biosciences | Creative Biolabs |
| | (Shirley, NY, | (San Diego, CA, | (Shirley, NY, |
| | USA) | USA) | USA) |
| Clone and | Urelumab; | Unknown; | Unknown; |
| Product | Catalog no. TAB- | Catalog no. | Catalog no. |
| Information | 179 | 79097-2, Lot no. | MOM-18455 |
| | | 170718 | |
| Isotype | IgG4 | IgG1 | IgG |
| Formulation | PBS | PBS and 20% | PBS |
| | | glycerol | |
| Purity | >95% | Unknown | >98% |
| Sequence | Available | Unknown | Unknown |
| and epitope | | | |
| Publication | N/A | Wilcox, R.A., et | N/A |
| | | al., J. Clin. Invest. | |
| | | 2002; Foell, J. et. | |
| | | al., J. Clin. Invest. | |
| | | 2003. | |

In addition to the monoclonal antibodies described above, the OX40 agonistic antibody clone Ber-ACT35 (BioLegend, San Diego, CA, USA) was also used in selected experiments described herein.

The overall experimental strategy included the following steps: reagent procurement and validation; ex vivo expansion experimental design; adding anti-4-1BB or anti-OX40 at day 0 of pre-REP experiments, using fresh melanoma, lung, cervical tumor samples; assessing the anti-OX40 in 21 mini-REP carried out on thawed head & neck, lung, melanoma, triple-negative breast cancer, and breast cancer pre-REP TIL samples; and assessment of TIL yield and cell lineage phenotype (CD4:CD8), T-cell subsets/extended phenotype, and functional assays.

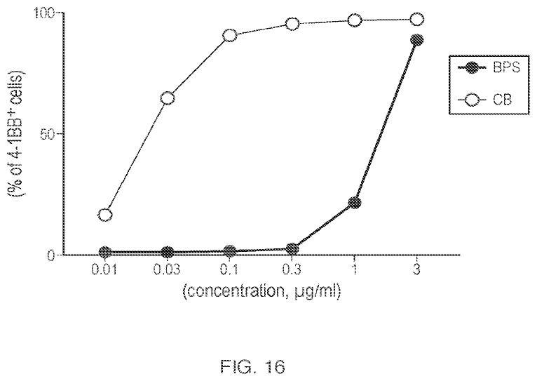

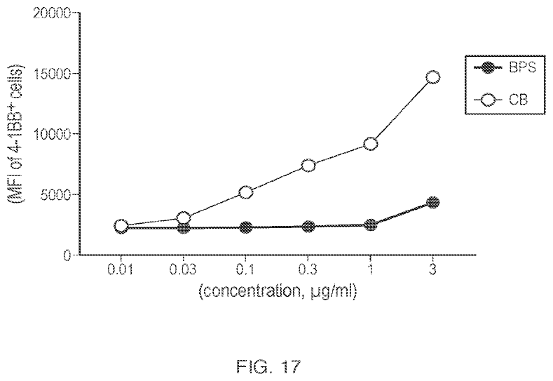

The comparability of anti-4-1BB binding affinity for two 4-1BB agonists was assessed. 4-1BB reporter cells were stained with anti-4-1BB antibody (Creative Biolabs) or anti-4-1BB (BPS Biosciences) at concentrations of 0.01, 0.03, 0.1, 0.3, 1, and 3 μg/ml together with FITC-conjugated mouse anti-human IgG and analyzed by flow cytometry. The results are shown in FIG. 16 and FIG. 17 (for % of 4-1BB⁺ cells and mean fluorescence intensity (MFI) of 4-1BB⁺ cells, respectively) and indicate that the Creative Biolabs (CB) 4-1BB urelumab antibody has the highest binding affinity.

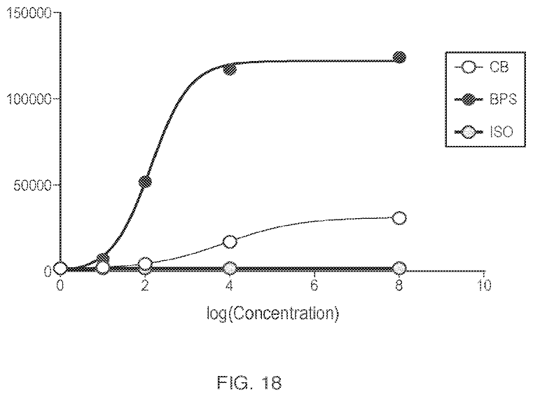

An assessment of NF-κB pathway activation of 4-1BB agonistic antibodies was also performed. 4-1BB reporter cells were treated with either anti-4-1BB (CB or BPS antibodies) at a concentration of 1, 2, 4, and 8 μg/mL for 24 hours. The cells were lysed using One-Step Luciferase reagent, and luciferase activity was measured by a luminometer. The results are shown in FIG. 18. Log EC50 for the CB antibody was determined to be 3.9 μg/mL and for the BPS antibody was determined to be 2.13 μg/mL. Both CB and BPS anti-4-1BB agonists had similar Log EC50 values even though the BPS antibody exhibited greater NF-kB signaling activation.

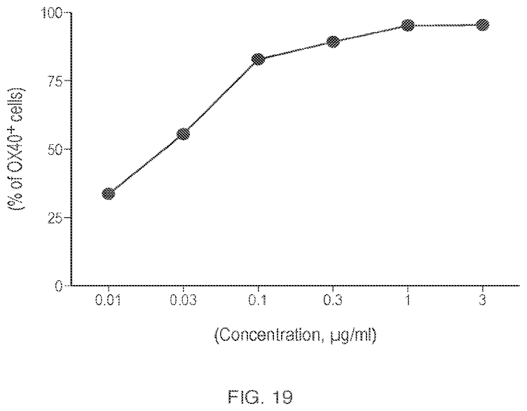

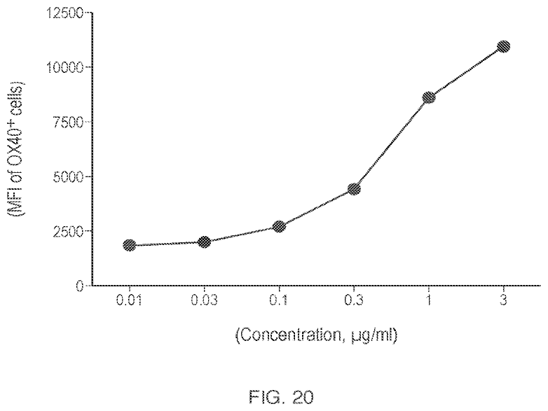

The binding affinity of the CB OX40 agonist was also assessed. OX40 reporter cells were stained with anti-OX40 Creative Biolabs (CB) agonist at the concentrations of 0.01, 0.03, 0.1, 0.3, 1, and 3 μg/ml together with FITC-conjugated mouse anti-human IgG and analyzed by flow cytometry. Results are shown in FIG. 19 and FIG. 20 for % of OX40⁺ cells and MFI of OX40⁺ cells, respectively, and indicate that the CB OX40 has a high binding affinity.

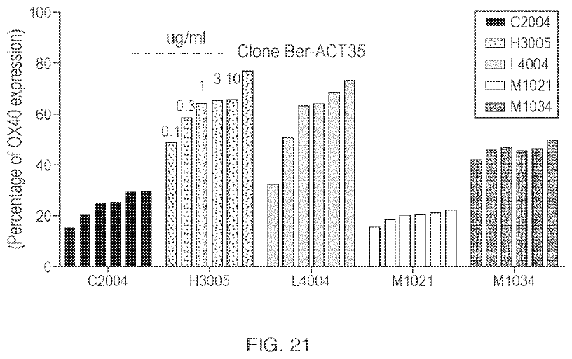

The comparability of OX40 binding affinity for two OX40 agonists, the CB OX40 agonist and the OX40 agonistic antibody clone Ber-ACT35 (BioLegend, San Diego, CA, USA), was assessed. Five different histologic TIL lines (including cervical, head and neck, lung, and melanoma) were stained with either anti-OX40 agonistic antibody at concentration of 0.1, 0.3, 1, 3, 10 (pg/mL) together with anti-human IgG secondary antibody or anti-OX40 (clone Ber-ACT35) alone. The results are shown in FIG. 21, and indicate comparable binding affinity for the two agonists.

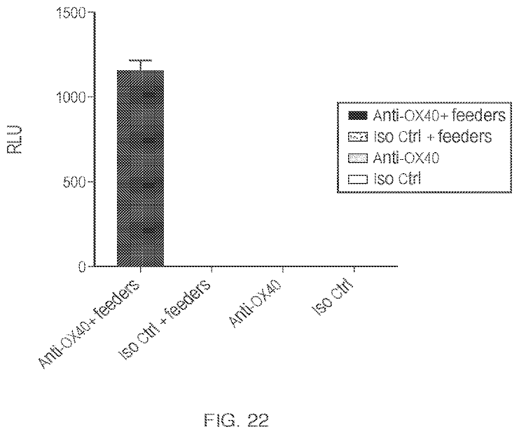

An assessment of NF-kB pathway activation of the CB OX40 agonist antibody was also performed, with results shown in FIG. 22. OX40 reporter cells were treated with either anti-OX40 alone or isotype control at concentrations of 1, 2, 4, 8, and 16 μg/mL with or without feeder cells for 24 hours. The cells were lysed using One-Step Luciferase reagent, and luciferase activity was measured by luminometer. The use of PBMC feeders initiated NF-κB activation using the OX40 reporter cell line, suggesting that clustering is involved in activation.

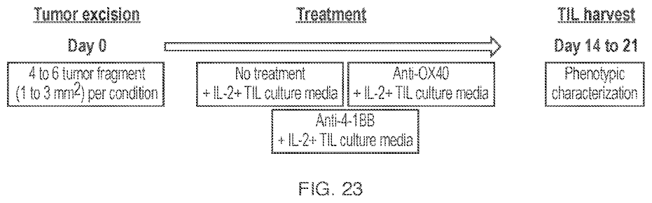

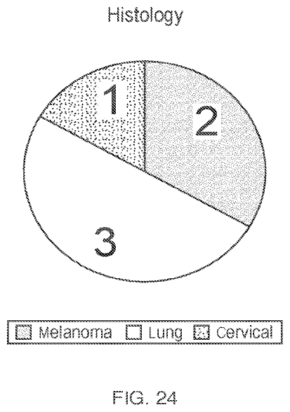

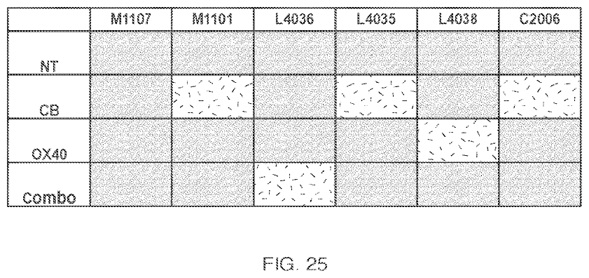

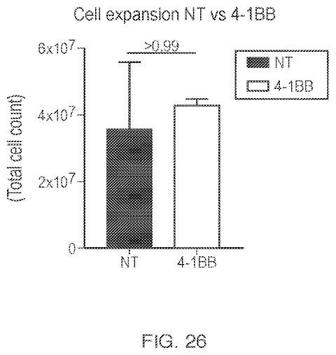

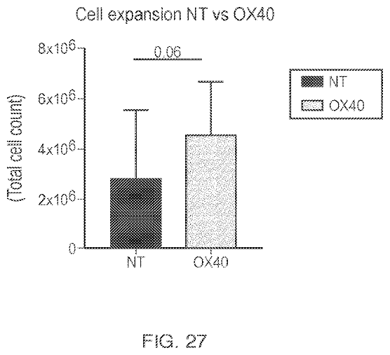

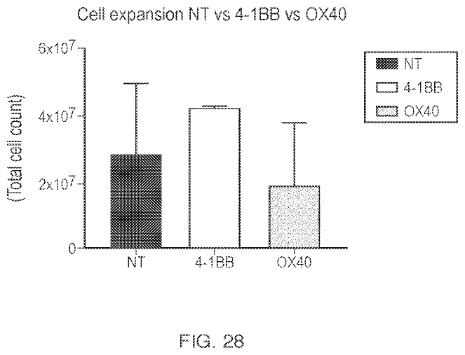

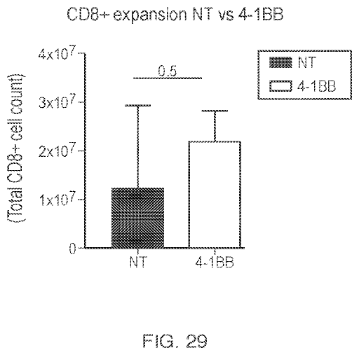

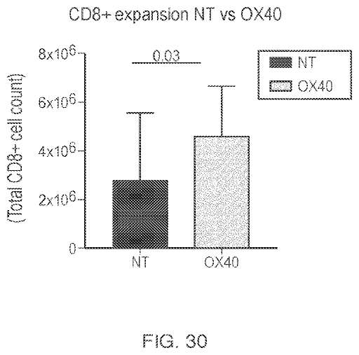

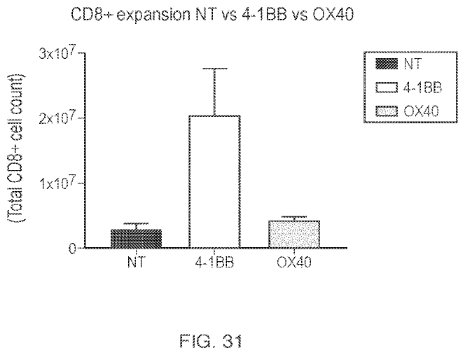

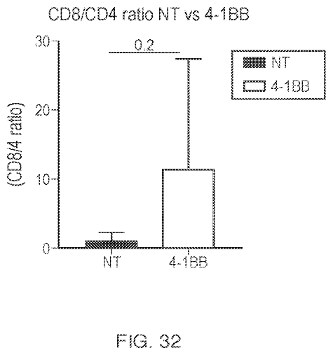

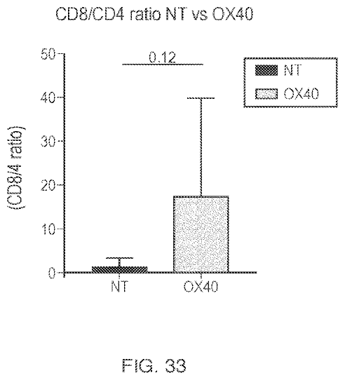

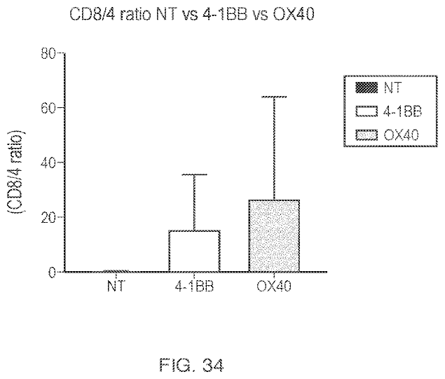

The experimental design for use of 4-1BB and OX40 agonists during the pre-REP step is shown in FIG. 23. The tumor histologies explored are shown in FIG. 24. The data analysis strategy is shown in FIG. 25. No treatment (IL-2 alone), anti-4-1BB, and anti-OX40 were analyzed in matched-pair manner. Using this approach, the samples were assigned into three different groups including: Group 1, No treatment and anti-4-1BB (n=3); Group 2, No treatment and anti-OX40 (n=5); and Group 3, No treatment and anti-4-1BB and anti-OX40 (n=2). Total cell count results from expansions are shown in FIG. 26 (CB 4-1BB agonist versus not tested, N=3); FIG. 27 (CB OX40 agonist versus not tested, N=5); and FIG. 28 (CB 4-1BB agonist and OX-40 agonist, N=2). CD8⁺ cell count results for cell expansion are shown in FIG. 29 (CB 4-1BB agonist versus not tested, N=3); FIG. 30 (CB OX40 agonist versus not tested, N=5); and FIG. 31 (CB 4-1BB agonist and OX-40 agonist, N=2). Total CD8⁺/CD4⁺ cell count ratio results for cell expansions are shown in FIG. 32 (CB 4-1BB agonist versus not tested, N=3); FIG. 33 (CB OX40 agonist versus not tested, N=5); and FIG. 34 (CB 4-1BB agonist and OX-40 agonist, N=2).

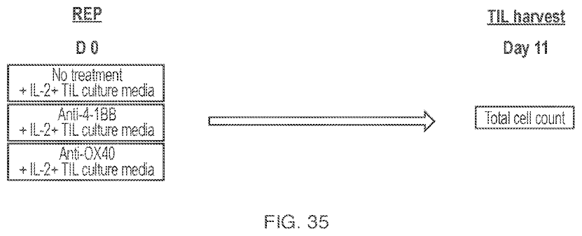

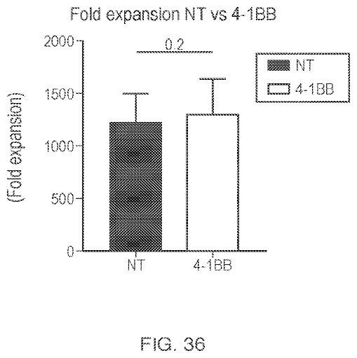

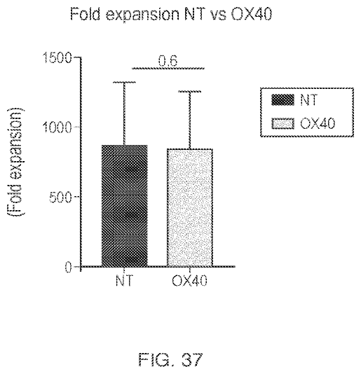

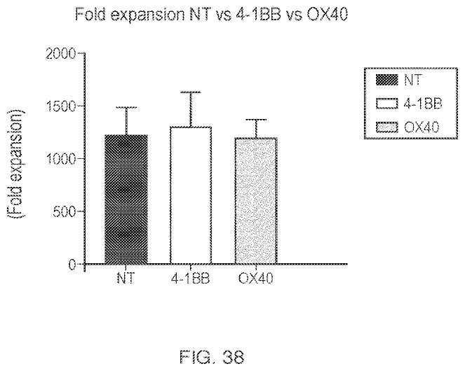

REP propagation of pre-REP TILs expanded in the presence of 4-1BB or OX40 agonists was also explored using the scheme shown in FIG. 35. Pre-REP TILs were expanded with either CB 4-1BB agonist or CB OX40 agonist were further propagated in a REP protocol in the presence of irradiated PBMCs, anti-CD3 antibody (30 ng/mL), and IL-2 (3000 IU/mL) for 11 days. TILs were harvested and counted, and fold expansion determined. Results are shown in FIG. 36, FIG. 37, and FIG. 38.

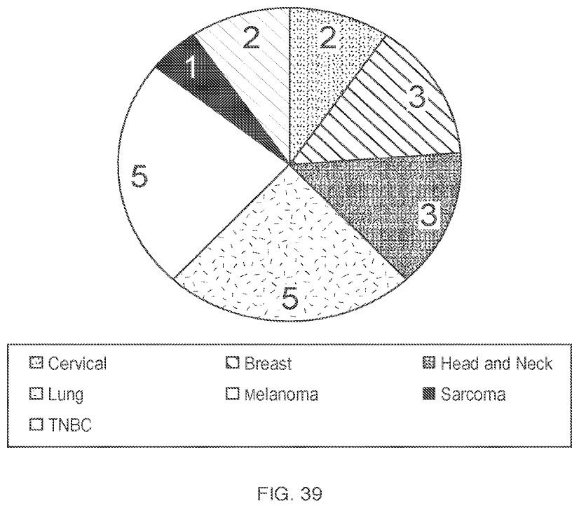

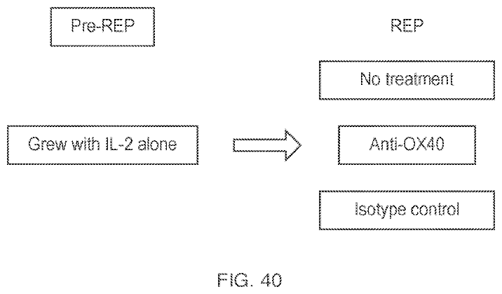

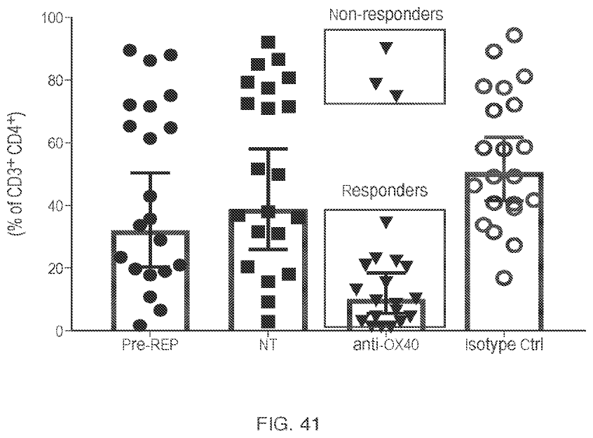

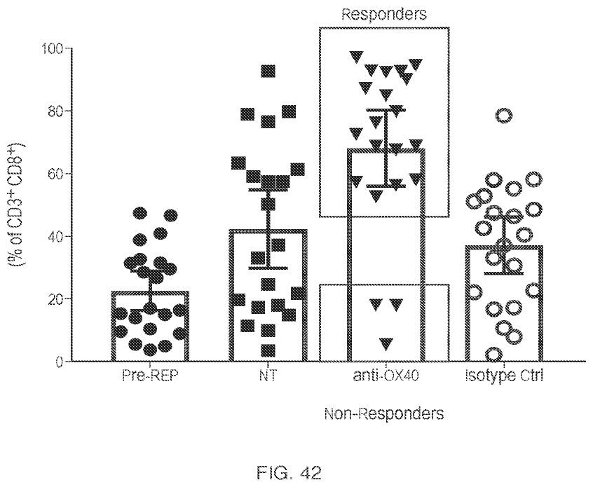

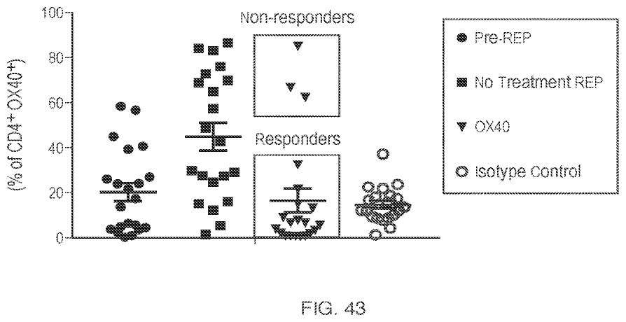

Assessment of OX40 during the REP phase was also tested. Twenty-one TIL lines from different histologies (FIG. 39) were propagated with REP with addition of CB OX40 agonist or isotype control antibody at concentration of 5 μg/mL. The experimental scheme is shown in FIG. 40. Results are shown in FIG. 41, FIG. 42, and FIG. 43. Surprisingly, the OX40 agonist preferentially expands CD8⁺ TILs during REP. TILs treated with OX40 agonist were classified as responders or non-responders.

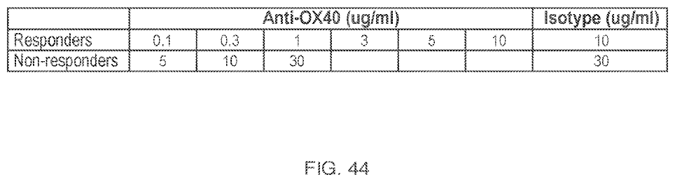

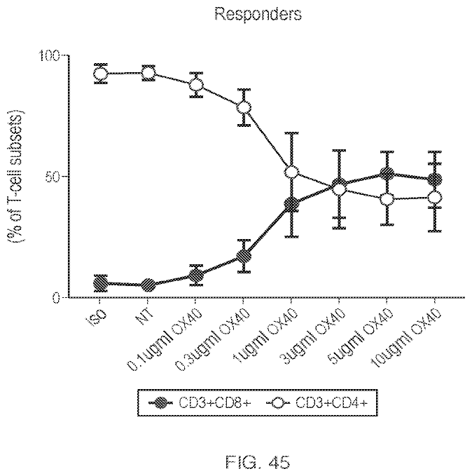

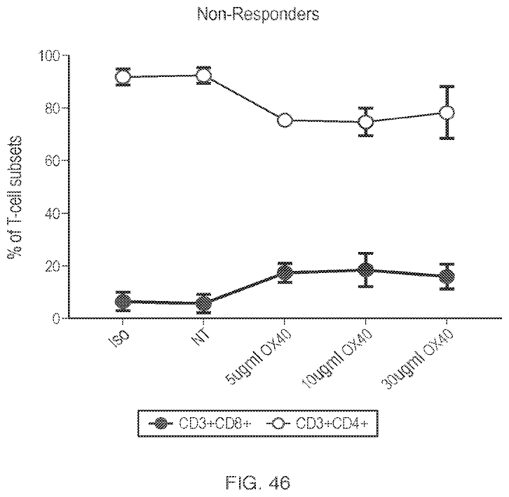

Anti-OX40 dose titration in non-responder and responder TIL lines was performed to further study this effect and to define the optimal concentration of OX40 agonist in responders and non-responders. TIL lines were categorized into two groups (responder and non-responder) based on enhanced CD8⁺ skewness following anti-OX40 treatment. Three non-responders (L4005, H3005, and M1022) and responders (T6001, T6003, and L4002) were propagated with REP in the presence of OX40 agonist or isotype control antibody following the conditions shown in FIG. 44. FIG. 45 and FIG. 46 illustrates that a dose-dependent manner of CD8⁺ skewness following anti-OX40 treatment was observed in responders, with concentrations in the 1 to 10 μg/mL range promoting skewness. Non-responders did not exhibit CD8⁺ skewness following anti-OX40 treatment even at high concentration (30 μg/mL).

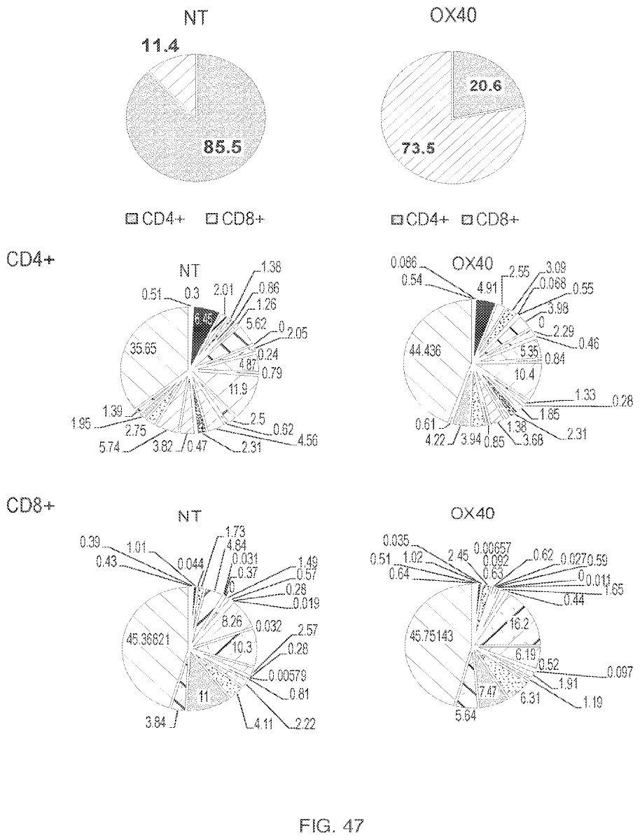

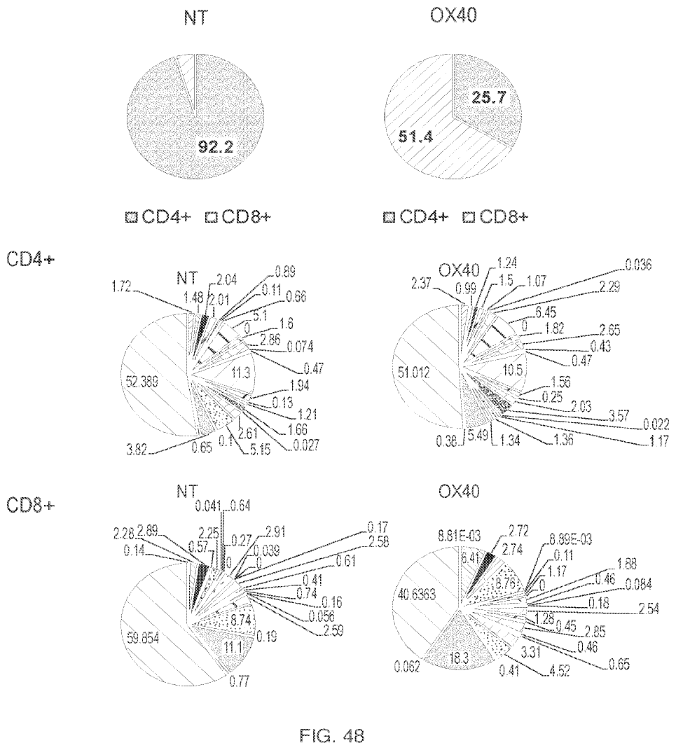

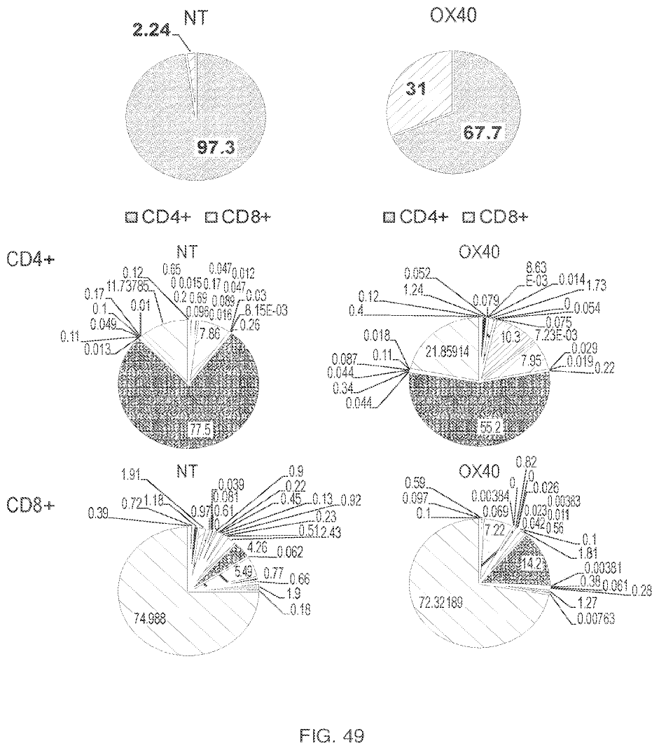

The impact of OX40 agonist on TCRvb repertoire in responders was also investigated. To determine whether anti-OX40, previously shown to skew CD8+ population, preferentially expand certain TCR vb repertoire. Responder TIL lines were propagated with REP in 24-well plates with either IL-2 alone or IL-2 with CD OX40 agonist monoclonal antibody (5 μg/mL). On day 11, TIL were harvested and stained with anti-CD3, anti-CD8, anti-CD4, and TCRvb repertoire antibodies, and analyzed by flow cytometry. Results are shown for three responders with three histologies in FIG. 47, FIG. 48, and FIG. 49. Minimal changes in TCRvb repertoire was observed, indicating that the high degree of polyclonality exhibited by the shortened 22-day process in an embodiment of FIG. 1 or FIG. 2 is surprisingly preserved in conjunction with the use of OX40 agonists during REP.

In conclusion, use of CB anti-OX40 antibody significantly enhanced pre-REP CD8⁺ TIL expansion, while use of CB anti-4-1BB antibody also demonstrated a promising trend. REP-fold expansion was comparable regardless of pre-treatment condition. Surprisingly, OX40 agonistic antibody increased CD8⁺/CD4⁺ ratio in REP TIL previously grown with IL-2 alone. In non-responder TILs, down-regulation of OX-40 was not observed in the CD4⁺ subset following anti-OX40 treatment. The dose-dependent manner of CD8⁺ skewness following anti-OX40 treatment was observed in responders. The change in TCRvb repertoire was very subtle even though significant CD8⁺ skewness was observed.

1-67. (canceled)

68. A process for the preparation of a population of tumor infiltrating lymphocytes (TILs) comprising the steps of:
(a) obtaining a first population of TILs;
(b) performing an initial expansion of the first population of TILs in a first cell culture medium to obtain a second population of TILs, wherein the first cell culture medium comprises IL-2 and a tumor necrosis factor receptor superfamily (TNFRSF) agonist, and wherein the initial expansion is performed over a period of 21 days or less;
(c) performing a rapid expansion of the second population of TILs in a second cell culture medium to obtain a third population of TILs, wherein the second cell culture medium comprises IL-2, OKT-3 (anti-CD3 antibody), peripheral blood mononuclear cells (PBMCs), and wherein the rapid expansion is performed over a period of 14 days or less; and
(d) harvesting the third population of TILs.

69. (canceled)

70. The process of claim 68, wherein the TNFRSF agonist is selected from the group consisting of a 4-1BB agonist, an OX40 agonist, a CD27 agonist, a GITR agonist, a HVEM agonist, a CD95 agonist, and combinations thereof.

71. The process of claim 68, wherein the TNFRSF agonist is a 4-1BB agonist.

72. The process of claim 68, the TNFRSF agonist is a 4-1BB agonist, and the 4-1BB agonist is selected from the group consisting of urelumab, utomilumab, and fragments, derivatives, variants, biosimilars, and combinations thereof.

73. The process of claim 68, wherein the TNFRSF agonist is a 4-1BB agonist, and the 4-1BB agonist is a 4-1BB agonist fusion protein.

74. The process of claim 68, wherein the TNFRSF agonist is a 4-1BB agonist fusion protein, and the 4-1BB agonist fusion protein comprises (i) a first soluble 4-1BB binding domain, (ii) a first peptide linker, (iii) a second soluble 4-1BB binding domain, (iv) a second peptide linker, and (v) a third soluble 4-1BB binding domain, further comprising an additional domain at the N-terminal and/or C-terminal end, and wherein the additional domain comprises a Fc fragment domain and hinge domain, and wherein the fusion protein is a dimeric structure according to structure I-A or structure I-B.

75. The process of claim 68, wherein the TNFRSF agonist is a OX40 agonist.

76. The process of claim 68, wherein the TNFRSF agonist is a OX40 agonist, and the OX40 agonist is selected from the group consisting of tavolixizumab, GSK3174998, MEDI6469, MEDI6383, MOXR0916, PF-04518600, Creative Biolabs MOM-18455, and fragments, derivatives, variants, biosimilars, and combinations thereof.

77. The process of claim 68, wherein the TNFRSF agonist is an OX40 agonist, and the OX40 agonist is an OX40 agonist fusion protein.

78. The process of claim 68, wherein the TNFRSF agonist is an OX40 agonist fusion protein, and the OX40 agonist fusion protein comprises (i) a first soluble OX40 binding domain, (ii) a first peptide linker, (iii) a second soluble OX40 binding domain, (iv) a second peptide linker, and (v) a third soluble OX40 binding domain, further comprising an additional domain at the N-terminal and/or C-terminal end, and wherein the additional domain comprises a Fc fragment domain and hinge domain, and wherein the fusion protein is a dimeric structure according to structure I-A or structure I-B.

79. The process of claim 68, wherein the TNFRSF agonist is a CD27 agonist.

80. The process of claim 68, wherein the TNFRSF agonist is a CD27 agonist, and the CD27 agonist is varlilumab, or a fragment, derivative, variant, or biosimilar thereof.

81. The process of claim 68, wherein the TNFRSF agonist is a CD27 agonist, and wherein the CD27 agonist is an CD27 agonist fusion protein.

82. The process of claim 68, wherein the TNFRSF agonist is a CD27 agonist, and the CD27 agonist fusion protein comprises (i) a first soluble CD27 binding domain, (ii) a first peptide linker, (iii) a second soluble CD27 binding domain, (iv) a second peptide linker, and (v) a third soluble CD27 binding domain, further comprising an additional domain at the N-terminal and/or C-terminal end, and wherein the additional domain comprises a Fc fragment domain and hinge domain, and wherein the fusion protein is a dimeric structure according to structure I-A or structure I-B.

83. The process of claim 68, wherein the TNFRSF agonist is a GITR agonist.

84. The process of claim 68, wherein the TNFRSF agonist is a GITR agonist, and the GITR agonist is selected from the group consisting of TRX518, 6C8, 36E5, 3D6, 61G6, 6H6, 61F6, 1D8, 17F10, 35D8, 49A1, 9E5, 31H6, 2155, 698, 706, 827, 1649, 1718, 1D7, 33C9, 33F6, 34G4, 35B10, 41E11, 41G5, 42A11, 44C1, 45A8, 46E11, 48H12, 48H7, 49D9, 49E2, 48A9, 5H7, 7A10, 9H6, and fragments, derivatives, variants, biosimilars, and combinations thereof.

85. The process of claim 68, wherein the TNFRSF agonist is an GITR agonist and the GITR agonist is a GITR agonist fusion protein.

86. The process of claim 68 to 85, wherein the TNFRSF agonist is a GITR agonist fusion protein, and the GITR agonist fusion protein comprises (i) a first soluble GITR binding domain, (ii) a first peptide linker, (iii) a second soluble GITR binding domain, (iv) a second peptide linker, and (v) a third soluble GITR binding domain, further comprising an additional domain at the N-terminal and/or C-terminal end, and wherein the additional domain comprises a Fc fragment domain and hinge domain, and wherein the fusion protein is a dimeric structure according to structure I-A or structure I-B.

87. The process of claim 68, wherein the TNFRSF agonist is a HVEM agonist.

88. The process of claim 68, wherein the TNFRSF agonist is an HVEM agonist, and the HVEM agonist is a HVEM agonist fusion protein.

89. The process of claim 68, wherein the TNFRSF agonist is a HVEM agonist fusion protein, and wherein the HVEM agonist fusion protein comprises (i) a first soluble HVEM binding domain, (ii) a first peptide linker, (iii) a second soluble HVEM binding domain, (iv) a second peptide linker, and (v) a third soluble HVEM binding domain, further comprising an additional domain at the N-terminal and/or C-terminal end, and wherein the additional domain comprises a Fc fragment domain and hinge domain, and wherein the fusion protein is a dimeric structure according to structure I-A or structure I-B.

90. The process of claim 68, wherein the TNFRSF agonist is selected from the group consisting of urelumab, utomilumab, tavolixizumab, Creative Biolabs MOM-18455, and fragments, derivatives, variants, biosimilars, and combinations thereof.

91-158. (canceled)


SEQUENCE LISTING (512 sequences)

SEQ ID NO: 1 - protein, 450 aa, synthetic construct
  REGION 1..450: note = Muromonab heavy chain
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGGTTGSSVTLGCLVKGYFPEPVTLTWNSGSLSSGVHTFPAVLQSDLYTLSSSVTVTSSTWPSQSITCNVAHPASSTKVDKKIEPRPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 213 aa, synthetic construct
  REGION 1..213: note = Muromonab light chain
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 134 aa, synthetic construct
  REGION 1..134: note = recombinant human IL-2 (rhIL-2)
MAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 4 - protein, 132 aa, synthetic construct
  REGION 1..132: note = Aldesleukin
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 5 - protein, 130 aa, synthetic construct
  REGION 1..130: note = recombinant human IL-4(rhIL-4)
MHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 6 - protein, 153 aa, synthetic construct
  REGION 1..153: note = recombinant human IL-7(rhIL-7)
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 7 - protein, 115 aa, synthetic construct
  REGION 1..115: note = recombinant human IL-15(rhIL-15)
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 8 - protein, 132 aa, synthetic construct
  REGION 1..132: note = recombinant human IL-21(rhIL-21)
MQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 9 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 10 - protein, 256 aa, Mus musculus
MGNNCYNVVVIVLLLVGCEKVGAVQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSFSPSTTISVTPEGGPGGHSLQVLTLFLALTSALLLALIFITLLFSVLKWIRKKFPHIFKQPFKKTTGAAQEEDACSCRCPQEEEGGGGGYEL

SEQ ID NO: 11 - protein, 441 aa, synthetic construct
  REGION 1..441: note = heavy chain for utomilumab
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 12 - protein, 214 aa, synthetic construct
  REGION 1..214: note = light chain for utomilumab
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 13 - protein, 116 aa, synthetic construct
  REGION 1..116: note = heavy chain variable region for utomilumab
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSS

SEQ ID NO: 14 - protein, 108 aa, synthetic construct
  REGION 1..108: note = light chain variable region for utomilumab
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVL

SEQ ID NO: 15 - protein, 6 aa, synthetic construct
  REGION 1..6: note = heavy chain CDR1 for utomilumab
STYWIS

SEQ ID NO: 16 - protein, 17 aa, synthetic construct
  REGION 1..17: note = heavy chain CDR2 for utomilumab
KIYPGDSYTNYSPSFQG

SEQ ID NO: 17 - protein, 8 aa, synthetic construct
  REGION 1..8: note = heavy chain CDR3 for utomilumab
RGYGIFDY

SEQ ID NO: 18 - protein, 11 aa, synthetic construct
  REGION 1..11: note = light chain CDR1 for utomilumab
SGDNIGDQYAH

SEQ ID NO: 19 - protein, 7 aa, synthetic construct
  REGION 1..7: note = light chain CDR2 for utomilumab
QDKNRPS

SEQ ID NO: 20 - protein, 11 aa, synthetic construct
  REGION 1..11: note = light chain CDR3 for utomilumab
ATYTGFGSLAV

SEQ ID NO: 21 - protein, 448 aa, synthetic construct
  REGION 1..448: note = heavy chain for urelumab
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 216 aa, synthetic construct
  REGION 1..216: note = light chain for urelumab
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFCGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 120 aa, synthetic construct
  REGION 1..120: note = variable heavy chain for urelumab
MKHLWFFLLLVAAPRWVLSQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGP

SEQ ID NO: 24 - protein, 110 aa, synthetic construct
  REGION 1..110: note = variable light chain for urelumab
MEAPAQLLFLLLLWLPDTTGEIVLTQSPATLSLSPGERATLSCRASQSVSSYLAWYQQKPGQAPRLLIYDASNRATGIPARFSGSGSGTDFTLTISSLEPEDFAVYYCQQ

SEQ ID NO: 25 - protein, 5 aa, synthetic construct
  REGION 1..5: note = heavy chain CDR1 for urelumab
GYYWS

SEQ ID NO: 26 - protein, 16 aa, synthetic construct
  REGION 1..16: note = heavy chain CDR2 for urelumab
EINHGGYVTYNPSLES

SEQ ID NO: 27 - protein, 13 aa, synthetic construct
  REGION 1..13: note = heavy chain CDR3 for urelumab
DYGPGNYDWYFDL

SEQ ID NO: 28 - protein, 11 aa, synthetic construct
  REGION 1..11: note = light chain CDR1 for urelumab
RASQSVSSYLA

SEQ ID NO: 29 - protein, 7 aa, synthetic construct
  REGION 1..7: note = light chain CDR2 for urelumab
DASNRAT

SEQ ID NO: 30 - protein, 11 aa, synthetic construct
  REGION 1..11: note = light chain CDR3 for urelumab
QQRSDWPPALT

SEQ ID NO: 31 - protein, 230 aa, synthetic construct
  REGION 1..230: note = Fc domain
KSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 22 aa, synthetic construct
  REGION 1..22: note = linker
GGPGSSKSCDKTHTCPPCPAPE

SEQ ID NO: 33 - protein, 22 aa, synthetic construct
  REGION 1..22: note = linker
GGSGSSKSCDKTHTCPPCPAPE

SEQ ID NO: 34 - protein, 27 aa, synthetic construct
  REGION 1..27: note = linker
GGPGSSSSSSSKSCDKTHTCPPCPAPE

SEQ ID NO: 35 - protein, 27 aa, synthetic construct
  REGION 1..27: note = linker
GGSGSSSSSSSKSCDKTHTCPPCPAPE

SEQ ID NO: 36 - protein, 29 aa, synthetic construct
  REGION 1..29: note = linker
GGPGSSSSSSSSSKSCDKTHTCPPCPAPE

SEQ ID NO: 37 - protein, 29 aa, synthetic construct
  REGION 1..29: note = linker
GGSGSSSSSSSSSKSCDKTHTCPPCPAPE

SEQ ID NO: 38 - protein, 24 aa, synthetic construct
  REGION 1..24: note = linker
GGPGSSGSGSSDKTHTCPPCPAPE

SEQ ID NO: 39 - protein, 23 aa, synthetic construct
  REGION 1..23: note = linker
GGPGSSGSGSDKTHTCPPCPAPE

SEQ ID NO: 40 - protein, 21 aa, synthetic construct
  REGION 1..21: note = linker
GGPSSSGSDKTHTCPPCPAPE

SEQ ID NO: 41 - protein, 25 aa, synthetic construct
  REGION 1..25: note = linker
GGSSSSSSSSGSDKTHTCPPCPAPE

SEQ ID NO: 42 - protein, 246 aa, synthetic construct
  REGION 1..246: note = Fc domain
METDTLLLWVLLLWVPAGNGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 11 aa, synthetic construct
  REGION 1..11: note = linker
SGSGSGSGSGS

SEQ ID NO: 44 - protein, 12 aa, synthetic construct
  REGION 1..12: note = linker
SSSSSSGSGSGS

SEQ ID NO: 45 - protein, 16 aa, synthetic construct
  REGION 1..16: note = linker
SSSSSSGSGSGSGSGS

SEQ ID NO: 46 - protein, 254 aa, synthetic construct
  REGION 1..254: note = 4-1BBL
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 47 - protein, 168 aa, synthetic construct
  REGION 1..168: note = 4-1BBL soluble domain
LRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 48 - protein, 118 aa, synthetic construct
  REGION 1..118: note = variable heavy chain for 4B4-1-1 version 1
QVQLQQPGAELVKPGASVKLSCKASGYTFSSYWMHWVKQRPGQVLEWIGEINPGNGHTNYNEKFKSKATLTVDKSSSTAYMQLSSLTSEDSAVYYCARSFTTARGFAYWGQGTLVTVS

SEQ ID NO: 49 - protein, 107 aa, synthetic construct
  REGION 1..107: note = variable light chain for 4B4-1-1 version 1
DIVMTQSPATQSVTPGDRVSLSCRASQTISDYLHWYQQKSHESPRLLIKYASQSISGIPSRFSGSGSGSDFTLSINSVEPEDVGVYYCQDGHSFPPTFGGGTKLEIK

SEQ ID NO: 50 - protein, 119 aa, synthetic construct
  REGION 1..119: note = variable heavy chain for 4B4-1-1 version 2
QVQLQQPGAELVKPGASVKLSCKASGYTFSSYWMHWVKQRPGQVLEWIGEINPGNGHTNYNEKFKSKATLTVDKSSSTAYMQLSSLTSEDSAVYYCARSFTTARGFAYWGQGTLVTVSA

SEQ ID NO: 51 - protein, 108 aa, synthetic construct
  REGION 1..108: note = variable light chain for 4B4-1-1 version 2
DIVMTQSPATQSVTPGDRVSLSCRASQTISDYLHWYQQKSHESPRLLIKYASQSISGIPSRFSGSGSGSDFTLSINSVEPEDVGVYYCQDGHSFPPTFGGGTKLEIKR

SEQ ID NO: 52 - protein, 120 aa, synthetic construct
  REGION 1..120: note = variable heavy chain for H39E3-2
MDWTWRILFLVAAATGAHSEVQLVESGGGLVQPGGSLRLSCAASGFTFSDYWMSWVRQAPGKGLEWVADIKNDGSYTNYAPSLTNRFTISRDNAKNSLYLQMNSLRAEDTAVYYCARELT

SEQ ID NO: 53 - protein, 109 aa, synthetic construct
  REGION 1..109: note = variable light chain for H39E3-2
MEAPAQLLFLLLLWLPDTTGDIVMTQSPDSLAVSLGERATINCKSSQSLLSSGNQKNYLWYQQKPGQPPKLLIYYASTRQSGVPDRFSGSGSGTDFTLTISSLQAEDVA

SEQ ID NO: 54 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 55 - protein, 272 aa, Mus musculus
MYVWVQQPTALLLLGLTLGVTARRLNCVKHTYPSGHKCCRECQPGHGMVSRCDHTRDTLCHPCETGFYNEAVNYDTCKQCTQCNHRSGSELKQNCTPTQDTVCRCRPGTQPRQDSGYKLGVDCVPCPPGHFSPGNNQACKPWTNCTLSGKQTRHPASDSLDAVCEDRSLLATLLWETQRPTFRPTTVQSTTVWPRTSELPSPPTLVTPEGPAFAVLLGLGLGLLAPLTVLLALYLLRKAWRLPNTPKPCWGNSFRTPIQEEHTDAHFTLAKI

SEQ ID NO: 56 - protein, 451 aa, synthetic construct
  REGION 1..451: note = heavy chain for tavolixizumab
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 214 aa, synthetic construct
  REGION 1..214: note = light chain for tavolixizumab
DIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 118 aa, synthetic construct
  REGION 1..118: note = heavy chain variable region for tavolixizumab
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVT

SEQ ID NO: 59 - protein, 108 aa, synthetic construct
  REGION 1..108: note = light chain variable region for tavolixizumab
DIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGQGTKVEIKR

SEQ ID NO: 60 - protein, 9 aa, synthetic construct
  REGION 1..9: note = heavy chain CDR1 for tavolixizumab
GSFSSGYWN

SEQ ID NO: 61 - protein, 13 aa, synthetic construct
  REGION 1..13: note = heavy chain CDR2 for tavolixizumab
YIGYISYNGITYH

SEQ ID NO: 62 - protein, 14 aa, synthetic construct
  REGION 1..14: note = heavy chain CDR3 for tavolixizumab
RYKYDYDGGHAMDY

SEQ ID NO: 63 - protein, 8 aa, synthetic construct
  REGION 1..8: note = light chain CDR1 for tavolixizumab
QDISNYLN

SEQ ID NO: 64 - protein, 11 aa, synthetic construct
  REGION 1..11: note = light chain CDR2 for tavolixizumab
LLIYYTSKLHS

SEQ ID NO: 65 - protein, 8 aa, synthetic construct
  REGION 1..8: note = light chain CDR3 for tavolixizumab
QQGSALPW

SEQ ID NO: 66 - protein, 444 aa, synthetic construct
  REGION 1..444: note = heavy chain for 11D4
EVQLVESGGGLVQPGGSLRLSCAASGFTFSSYSMNWVRQAPGKGLEWVSYISSSSSTIDYADSVKGRFTISRDNAKNSLYLQMNSLRDEDTAVYYCARESGWYLFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 67 - protein, 214 aa, synthetic construct
  REGION 1..214: note = light chain for 11D4
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 118 aa, synthetic construct
  REGION 1..118: note = heavy chain variable region for 11D4
EVQLVESGGGLVQPGGSLRLSCAASGFTFSSYSMNWVRQAPGKGLEWVSYISSSSSTIDYADSVKGRFTISRDNAKNSLYLQMNSLRDEDTAVYYCARESGWYLFDYWGQGTLVTVSS

SEQ ID NO: 69 - protein, 107 aa, synthetic construct
  REGION 1..107: note = light chain variable region for 11D4
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGGGTKVEIK

SEQ ID NO: 70 - protein, 5 aa, synthetic construct
  REGION 1..5: note = heavy chain CDR1 for 11D4
SYSMN

SEQ ID NO: 71 - protein, 17 aa, synthetic construct
  REGION 1..17: note = heavy chain CDR2 for 11D4
YISSSSSTIDYADSVKG

SEQ ID NO: 72 - protein, 9 aa, synthetic construct
  REGION 1..9: note = heavy chain CDR3 for 11D4
ESGWYLFDY

SEQ ID NO: 73 - protein, 11 aa, synthetic construct
  REGION 1..11: note = light chain CDR1 for 11D4
RASQGISSWLA

SEQ ID NO: 74 - protein, 7 aa, synthetic construct
  REGION 1..7: note = light chain CDR2 for 11D4
AASSLQS

SEQ ID NO: 75 - protein, 9 aa, synthetic construct
  REGION 1..9: note = light chain CDR3 for 11D4
QQYNSYPPT

SEQ ID NO: 76 - protein, 450 aa, synthetic construct
  REGION 1..450: note = heavy chain for 18D8
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDQSTADYYFYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 77 - protein, 213 aa, synthetic construct
  REGION 1..213: note = light chain for 18D8
EIVVTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 124 aa, synthetic construct
  REGION 1..124: note = heavy chain variable region for 18D8
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDQSTADYYFYYGMDVWGQGTTVTVSS

SEQ ID NO: 79 - protein, 106 aa, synthetic construct
  REGION 1..106: note = light chain variable region for 18D8
EIVVTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 80 - protein, 5 aa, synthetic construct
  REGION 1..5: note = heavy chain CDR1 for 18D8
DYAMH

SEQ ID NO: 81 - protein, 17 aa, synthetic construct
  REGION 1..17: note = heavy chain CDR2 for 18D8
GISWNSGSIGYADSVKG

SEQ ID NO: 82 - protein, 15 aa, synthetic construct
  REGION 1..15: note = heavy chain CDR3 for 18D8
DQSTADYYFYYGMDV

SEQ ID NO: 83 - protein, 11 aa, synthetic construct
  REGION 1..11: note = light chain CDR1 for 18D8
RASQSVSSYLA

SEQ ID NO: 84 - protein, 7 aa, synthetic construct
  REGION 1..7: note = light chain CDR2 for 18D8
DASNRAT

SEQ ID NO: 85 - protein, 8 aa, synthetic construct
  REGION 1..8: note = light chain CDR3 for 18D8
QQRSNWPT

SEQ ID NO: 86 - protein, 120 aa, synthetic construct
  REGION 1..120: note = heavy chain variable region for Hu119-122
EVQLVESGGGLVQPGGSLRLSCAASEYEFPSHDMSWVRQAPGKGLELVAAINSDGGSTYYPDTMERRFTISRDNAKNSLYLQMNSLRAEDTAVYYCARHYDDYYAWFAYWGQGTMVTVSS

SEQ ID NO: 87 - protein, 111 aa, synthetic construct
  REGION 1..111: note = light chain variable region for Hu119-122
EIVLTQSPATLSLSPGERATLSCRASKSVSTSGYSYMHWYQQKPGQAPRLLIYLASNLESGVPARFSGSGSGTDFTLTISSLEPEDFAVYYCQHSRELPLTFGGGTKVEIK

SEQ ID NO: 88 - protein, 5 aa, synthetic construct
  REGION 1..5: note = heavy chain CDRl for Hu119-122
SHDMS

SEQ ID NO: 89 - protein, 17 aa, synthetic construct
  REGION 1..17: note = heavy chain CDR2 for Hu119-122
AINSDGGSTYYPDTMER

SEQ ID NO: 90 - protein, 11 aa, synthetic construct
  REGION 1..11: note = heavy chain CDR3 for Hu119-122
HYDDYYAWFAY

SEQ ID NO: 91 - protein, 15 aa, synthetic construct
  REGION 1..15: note = light chain CDR1 for Hu119-122
RASKSVSTSGYSYMH

SEQ ID NO: 92 - protein, 7 aa, synthetic construct
  REGION 1..7: note = light chain CDR2 for Hu119-122
LASNLES

SEQ ID NO: 93 - protein, 9 aa, synthetic construct
  REGION 1..9: note = light chain CDR3 for Hu119-122
QHSRELPLT

SEQ ID NO: 94 - protein, 122 aa, synthetic construct
  REGION 1..122: note = heavy chain variable region for Hu106-222
QVQLVQSGSELKKPGASVKVSCKASGYTFTDYSMHWVRQAPGQGLKWMGWINTETGEPTYADDFKGRFVFSLDTSVSTAYLQISSLKAEDTAVYYCANPYYDYVSYYAMDYWGQGTTVTVSS

SEQ ID NO: 95 - protein, 107 aa, synthetic construct
  REGION 1..107: note = light chain variable region for Hu106-222
DIQMTQSPSSLSASVGDRVTITCKASQDVSTAVAWYQQKPGKAPKLLIYSASYLYTGVPSRFSGSGSGTDFTFTISSLQPEDIATYYCQQHYSTPRTFGQGTKLEIK

SEQ ID NO: 96 - protein, 5 aa, synthetic construct
  REGION 1..5: note = heavy chain CDR1 for Hu106-222
DYSMH

SEQ ID NO: 97 - protein, 17 aa, synthetic construct
  REGION 1..17: note = heavy chain CDR2 for Hu106-222
WINTETGEPTYADDFKG

SEQ ID NO: 98 - protein, 13 aa, synthetic construct
  REGION 1..13: note = heavy chain CDR3 for Hu106-222
PYYDYVSYYAMDY

SEQ ID NO: 99 - protein, 11 aa, synthetic construct
  REGION 1..11: note = light chain CDR1 for Hu106-222
KASQDVSTAVA

SEQ ID NO: 100 - protein, 7 aa, synthetic construct
  REGION 1..7: note = light chain CDR2 for Hu106-222
SASYLYT

SEQ ID NO: 101 - protein, 9 aa, synthetic construct
  REGION 1..9: note = light chain CDR3 for Hu106-222
QQHYSTPRT

SEQ ID NO: 102 - protein, 183 aa, synthetic construct
  REGION 1..183: note = OX40L
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 103 - protein, 131 aa, synthetic construct
  REGION 1..131: note = OX40L soluble domain
SHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 104 - protein, 128 aa, synthetic construct
  REGION 1..128: note = OX40L soluble domain (alternative)
YPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 105 - protein, 120 aa, synthetic construct
  REGION 1..120: note = variable heavy chain for 008
EVQLVESGGGLVQPGGSLRLSCAASGFTFSNYTMNWVRQAPGKGLEWVSAISGSGGSTYYADSVKGRFTISRDNSKNTLYLQMNSLRAEDTAVYYCAKDRYSQVHYALDYWGQGTLVTVS

SEQ ID NO: 106 - protein, 108 aa, synthetic construct
  REGION 1..108: note = variable light chain for 008
DIVMTQSPDSLPVTPGEPASISCRSSQSLLHSNGYNYLDWYLQKAGQSPQLLIYLGSNRASGVPDRFSGSGSGTDFTLKISRVEAEDVGVYYCQQYYNHPTTFGQGTK

SEQ ID NO: 107 - protein, 120 aa, synthetic construct
  REGION 1..120: note = variable heavy chain for 011
EVQLVESGGGVVQPGRSLRLSCAASGFTFSDYTMNWVRQAPGKGLEWVSSISGGSTYYADSRKGRFTISRDNSKNTLYLQMNNLRAEDTAVYYCARDRYFRQQNAFDYWGQGTLVTVSSA

SEQ ID NO: 108 - protein, 108 aa, synthetic construct; identical to SEQ ID NO: 106
  REGION 1..108: note = variable light chain for 011

SEQ ID NO: 109 - protein, 120 aa, synthetic construct
  REGION 1..120: note = variable heavy chain for 021
EVQLVESGGGLVQPRGSLRLSCAASGFTFSSYAMNWVRQAPGKGLEWVAVISYDGSNKYYADSVKGRFTISRDNSKNTLYLQMNSLRAEDTAVYYCAKDRYITLPNALDYWGQGTLVTVS

SEQ ID NO: 110 - protein, 108 aa, synthetic construct
  REGION 1..108: note = variable light chain for 021
DIQMTQSPVSLPVTPGEPASISCRSSQSLLHSNGYNYLDWYLQKPGQSPQLLIYLGSNRASGVPDRFSGSGSGTDFTLKISRVEAEDVGVYYCQQYKSNPPTFGQGTK

SEQ ID NO: 111 - protein, 120 aa, synthetic construct
  REGION 1..120: note = variable heavy chain for 023
EVQLVESGGGLVHPGGSLRLSCAGSGFTFSSYAMHWVRQAPGKGLEWVSAIGTGGGTYYADSVMGRFTISRDNSKNTLYLQMNSLRAEDTAVYYCARYDNVMGLYWFDYWGQGTLVTVSS

SEQ ID NO: 112 - protein, 108 aa, synthetic construct
  REGION 1..108: note = variable light chain for 023
EIVLTQSPATLSLSPGERATLSCRASQSVSSYLAWYQQKPGQAPRLLIYDASNRATGIPARFSGSGSGTDFTLTISSLEPEDFAVYYCQQRSNWPPAFGGGTKVEIKR

SEQ ID NO: 113 - protein, 119 aa, synthetic construct
  REGION 1..119: note = heavy chain variable region
EVQLQQSGPELVKPGASVKMSCKASGYTFTSYVMHWVKQKPGQGLEWIGYINPYNDGTKYNEKFKGKATLTSDKSSSTAYMELSSLTSEDSAVYYCANYYGSSLSMDYWGQGTSVTVSS

SEQ ID NO: 114 - protein, 108 aa, synthetic construct
  REGION 1..108: note = light chain variable region
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIKR

SEQ ID NO: 115 - protein, 121 aa, synthetic construct
  REGION 1..121: note = heavy chain variable region
EVQLQQSGPELVKPGASVKISCKTSGYTFKDYTMHWVKQSHGKSLEWIGGIYPNNGGSTYNQNFKDKATLTVDKSSSTAYMEFRSLTSEDSAVYYCARMGYHGPHLDFDVWGAGTTVTVSP

SEQ ID NO: 116 - protein, 108 aa, synthetic construct
  REGION 1..108: note = light chain variable region
DIVMTQSHKFMSTSLGDRVSITCKASQDVGAAVAWYQQKPGQSPKLLIYWASTRHTGVPDRFTGGGSGTDFTLTISNVQSEDLTDYFCQQYINYPLTFGGGTKLEIKR

SEQ ID NO: 117 - protein, 122 aa, synthetic construct
  REGION 1..122: note = heavy chain variable region of humanized antibody
QIQLVQSGPELKKPGETVKISCKASGYTFTDYSMHWVKQAPGKGLKWMGWINTETGEPTYADDFKGRFAFSLETSASTAYLQINNLKNEDTATYFCANPYYDYVSYYAMDYWGHGTSVTVSS

SEQ ID NO: 118 - protein, 122 aa, synthetic construct; identical to SEQ ID NO: 94
  REGION 1..122: note = heavy chain variable region of humanized antibody

SEQ ID NO: 119 - protein, 107 aa, synthetic construct
  REGION 1..107: note = light chain variable region of humanized antibody
DIVMTQSHKFMSTSVRDRVSITCKASQDVSTAVAWYQQKPGQSPKLLIYSASYLYTGVPDRFTGSGSGTDFTFTISSVQAEDLAVYYCQQHYSTPRTFGGGTKLEIK

SEQ ID NO: 120 - protein, 107 aa, synthetic construct; identical to SEQ ID NO: 119
  REGION 1..107: note = light chain variable region of humanized antibody

SEQ ID NO: 121 - protein, 120 aa, synthetic construct
  REGION 1..120: note = heavy chain variable region of humanized antibody
EVQLVESGGGLVQPGESLKLSCESNEYEFPSHDMSWVRKTPEKRLELVAAINSDGGSTYYPDTMERRFIISRDNTKKTLYLQMSSLRSEDTALYYCARHYDDYYAWFAYWGQGTLVTVSA

SEQ ID NO: 122 - protein, 120 aa, synthetic construct; identical to SEQ ID NO: 86
  REGION 1..120: note = heavy chain variable region of humanized antibody

SEQ ID NO: 123 - protein, 111 aa, synthetic construct
  REGION 1..111: note = light chain variable region of humanized antibody
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPLTFGAGTKLELK

SEQ ID NO: 124 - protein, 111 aa, synthetic construct; identical to SEQ ID NO: 87
  REGION 1..111: note = light chain variable region of humanized antibody

SEQ ID NO: 125 - protein, 138 aa, synthetic construct
  REGION 1..138: note = heavy chain variable region
MYLGLNYVFIVFLLNGVQSEVKLEESGGGLVQPGGSMKLSCAASGFTFSDAWMDWVRQSPEKGLEWVAEIRSKANNHATYYAESVNGRFTISRDDSKSSVYLQMNSLRAEDTGIYYCTWGEVFYFDYWGQGTTLTVSS

SEQ ID NO: 126 - protein, 126 aa, synthetic construct
  REGION 1..126: note = light chain variable region
MRPSIQFLGLLLFWLHGAQCDIQMTQSPSSLSASLGGKVTITCKSSQDINKYIAWYQHKPGKGPRLLIHYTSTLQPGIPSRFSGSGSGRDYSFSISNLEPEDIATYYCLQYDNLLTFGAGTKLELK

SEQ ID NO: 127 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCRYSCPREEEGSTIPIQEDYRKPEPACSP

SEQ ID NO: 128 - protein, 260 aa, Macaca nemestrina
MARPHPWWLCFLGTLVGLSATPAPKSCPERHYWAQGKLCCQMCEPGTFLVKDCDQHRKAAQCHPCIPGVSFSPDHHTRPHCESCRHCNSGLLIRNCTITANAVCACRNGWQCRDKECTECDPPPNPSLTTWPSQALGPHPQPTHLPYVNEMLEARTAGHMQTLADFRHLPARTLSTHWPPQRSLCSSDFIRILVIFSGMFLVFTLAGTLFLHQQRKYRSNKGESPMEPAEPCPYSCPREEEGSTIPIQEDYRKPEPASSP

SEQ ID NO: 129 - protein, 452 aa, synthetic construct
  REGION 1..452: note = heavy chain for varlilumab
QVQLVESGGGVVQPGRSLRLSCAASGFTFSSYDMHWVRQAPGKGLEWVAVIWYDGSNKYYADSVKGRFTISRDNSKNTLYLQMNSLRAEDTAVYYCARGSGNWGFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSS

SEQ ID NO: 130 - protein, 214 aa, synthetic construct
  REGION 1..214: note = light chain for varlilumab
DIQMTQSPSSLSASVGDRVTITCRASQGISRWLAWYQQKPEKAPKSLIYAASSLQSGVPSRFSGSGSGTDFTLTISSLQPEDFATYYCQQYNTYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 119 aa, synthetic construct
  REGION 1..119: note = heavy chain variable region for varlilumab
QVQLVESGGGVVQPGRSLRLSCAASGFTFSSYDMHWVRQAPGKGLEWVAVIWYDGSNKYYADSVKGRFTISRDNSKNTLYLQMNSLRAEDTAVYYCARGSGNWGFFDYWGQGTLVTVSS

SEQ ID NO: 132 - protein, 107 aa, synthetic construct
  REGION 1..107: note = light chain variable region for varlilumab
DIQMTQSPSSLSASVGDRVTITCRASQGISRWLAWYQQKPEKAPKSLIYAASSLQSGVPSRFSGSGSGTDFTLTISSLQPEDFATYYCQQYNTYPRTFGQGTKVEIK

SEQ ID NO: 133 - protein, 8 aa, synthetic construct
  REGION 1..8: note = heavy chain CDR1 for varlilumab
GFTFSSYD

SEQ ID NO: 134 - protein, 8 aa, synthetic construct
  REGION 1..8: note = heavy chain CDR2 for varlilumab
IWYDGSNK

SEQ ID NO: 135 - protein, 12 aa, synthetic construct
  REGION 1..12: note = heavy chain CDR3 for varlilumab
ARGSGNWGFFDY

SEQ ID NO: 136 - protein, 6 aa, synthetic construct
  REGION 1..6: note = light chain CDR1 for varlilumab
QGISRW

SEQ ID NO: 137 - protein, 4 aa, synthetic construct
  REGION 1..4: note = light chain CDR2 for varlilumab
AASG

SEQ ID NO: 138 - protein, 9 aa, synthetic construct
  REGION 1..9: note = light chain CDR3 for varlilumab
QQYNTYPRT

SEQ ID NO: 139 - protein, 193 aa, synthetic construct
  REGION 1..193: note = CD70 (CD27L)
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 140 - protein, 142 aa, synthetic construct
  REGION 1..142: note = CD70 soluble domain
SLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 141 - protein, 137 aa, synthetic construct
  REGION 1..137: note = CD70 soluble domain (alternative)
VAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 142 - protein, 241 aa, Homo sapiens
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRSQCMWPRETQLLLEVPPSTEDARSCQFPEEERGERSAEEKGRLGDLWV

SEQ ID NO: 143 - protein, 228 aa, Mus musculus
MGAWAMLYGVSMLCVLDLGQPSVVEEPGCGPGKVQNGSGNNTRCCSLYAPGKEDCPKERCICVTPEYHCGDPQCKICKHYPCQPGQRVESQGDIVFGFRCVACAMGTFSAGRDGHCRLWTNCSQFGFLTMFPGNKTHNAVCIPEPLPTEQYGHLTVIFLVMAACIFFLTTVQLGLHIWQLRRQHMCPRETQPFAEVQLSAEDACSFQFPEEERGEQTEEKCHLGGRWP

SEQ ID NO: 144 - protein, 449 aa, synthetic construct
  REGION 1..449: note = humanized 6C8 heavy chain variant
QVTLRESGPALVKPTQTLTLTCTFSGFSLSTSGMGVGWIRQPPGKALEWLAHIWWDDDKYYNPSLKSRLTISKDTSKNQVVLTMTNMDPVDTATYYCARTRRYFPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 449 aa, synthetic construct
  REGION 1..449: note = humanized 6C8 heavy chain variant
QVTLRESGPALVKPTQTLTLTCTFSGFSLSTSGMGVGWIRQPPGKALEWLAHIWWDDDKYYNPSLKSRLTISKDTSKNQVVLTMTNMDPVDTATYYCARTRRYFPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 449 aa, synthetic construct
  REGION 1..449: note = humanized 6C8 heavy chain variant
QVTLRESGPALVKPTQTLTLTCTFSGFSLSTSGMGVGWIRQPPGKALEWLAHIWWDDDKYYQPSLKSRLTISKDTSKNQVVLTMTNMDPVDTATYYCARTRRYFPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 449 aa, synthetic construct
  REGION 1..449: note = humanized 6C8 heavy chain variant
QVTLRESGPALVKPTQTLTLTCTFSGFSLSTSGMGVGWIRQPPGKALEWLAHIWWDDDKYYQPSLKSRLTISKDTSKNQVVLTMTNMDPVDTATYYCARTRRYFPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 214 aa, synthetic construct
  REGION 1..214: note = humanized 6C8 light chain
EIVMTQSPATLSVSPGERATLSCKASQNVGTNVAWYQQKPGQAPRLLIYSASYRYSGIPARFSGSGSGTEFTLTISSLQSEDFAVYYCQQYNTDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 149 - protein, 19 aa, synthetic construct
  REGION 1..19: note = 6C8 heavy chain leader
MDRLTFSFLLLIVPAYVLS

SEQ ID NO: 150 - protein, 20 aa, synthetic construct
  REGION 1..20: note = 6C8 light chain leader
METQSQVFVYMLLWLSGVDG

SEQ ID NO: 151 - protein, 138 aa, synthetic construct
  REGION 1..138: note = humanized 6C8 heavy chain variable region variant
MDRLTFSFLLLIVPAYVLSQVTLKESGPGILKPSQTLSLTCSFSGFSLSTSGMGVGWIRQPSGKGLEWLAHIWWDDDKYYNPSLKSQLTISKDTSRNQVFLKITSVDTADAATYYCARTRRYFPFAYWGQGTLVTVSS

SEQ ID NO: 152 - protein, 138 aa, synthetic construct
  REGION 1..138: note = humanized 6C8 heavy chain variable region variant
MDRLTFSFLLLIVPAYVLSQVTLKESGPGILKPSQTLSLTCSFSGFSLSTSGMGVGWIRQPSGKGLEWLAHIWWDDDKYYQPSLKSQLTISKDTSRNQVFLKITSVDTADAATYYCARTRRYFPFAYWGQGTLVTVSS

SEQ ID NO: 153 - protein, 127 aa, synthetic construct
  REGION 1..127: note = humanized 6C8 light chain variable region
METQSQVFVYMLLWLSGVDGDIVMTQSQKFMSTSVGDRVSVTCKASQNVGTNVAWYQQKPGQSPKALIYSASYRYSGVPDRFTGSGSGTDFTLTINNVHSEDLAEYFCQQYNTDPLTFGAGTKLEIK

SEQ ID NO: 154 - protein, 12 aa, synthetic construct
  REGION 1..12: note = 6C8 heavy chain CDR1
GFSLSTSGMGVG

SEQ ID NO: 155 - protein, 16 aa, synthetic construct
  REGION 1..16: note = 6C8 heavy chain CDR2 variant
HIWWDDDKYYNPSLKS

SEQ ID NO: 156 - protein, 16 aa, synthetic construct
  REGION 1..16: note = 6C8 heavy chain CDR2 variant
HIWWDDDKYYQPSLKS

SEQ ID NO: 157 - protein, 9 aa, synthetic construct
  REGION 1..9: note = 6C8 heavy chain CDR3
TRRYFPFAY

SEQ ID NO: 158 - protein, 11 aa, synthetic construct
  REGION 1..11: note = 6C8 light chain CDR1
KASQNVGTNVA

SEQ ID NO: 159 - protein, 7 aa, synthetic construct
  REGION 1..7: note = 6C8 light chain CDR2
SASYRYS

SEQ ID NO: 160 - protein, 9 aa, synthetic construct
  REGION 1..9: note = 6C8 light chain CDR3
QQYNTDPLT

SEQ ID NO: 161 - protein, 449 aa, synthetic construct
  REGION 1..449: note = chimeric 6C8 heavy chain variant
QVTLKESGPGILKPSQTLSLTCSFSGFSLSTSGMGVGWIRQPSGKGLEWLAHIWWDDDKYYNPSLKSQLTISKDTSRNQVFLKITSVDTADAATYYCARTRRYFPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 449 aa, synthetic construct
  REGION 1..449: note = chimeric 6C8 heavy chain variant
QVTLKESGPGILKPSQTLSLTCSFSGFSLSTSGMGVGWIRQPSGKGLEWLAHIWWDDDKYYNPSLKSQLTISKDTSRNQVFLKITSVDTADAATYYCARTRRYFPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 214 aa, synthetic construct
  REGION 1..214: note = chimeric 6C8 light chain variant
DIVMTQSQKFMSTSVGDRVSVTCKASQNVGTNVAWYQQKPGQSPKALIYSASYRYSGVPDRFTGSGSGTDFTLTINNVHSEDLAEYFCQQYNTDPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 118 aa, synthetic construct
  REGION 1..118: note = 36E5 heavy chain variable region
EVNLVESGGGLVKPGGSLKVSCAASGFTFSSYAMSWVRQTPEKRLEWVASISSGGTTYYPDSVKGRFTISRDNARNILYLQMSSLRSEDTAMYYCARVGGYYDSMDYWGQGISVTDSS

SEQ ID NO: 165 - protein, 113 aa, synthetic construct
  REGION 1..113: note = 36E5 light chain variable region
DIVLTQSPASLAVSLGQRATISCRASESVDNYGVSFMNWFQQKPGQPPKLLIYAASNQGSGVPARFSGSGSGTDFSLNIHPMEEDDTAMYFCQQTKEVTWTFGGGTKLEIKRA

SEQ ID NO: 166 - protein, 123 aa, synthetic construct
  REGION 1..123: note = 3D6 heavy chain variable region
EVQLVESGGGLVQPGRSLKLSCAASGFTFSDYYMAWVRQAPTKGLEWVAYIHANGGSTYYRDSVRGRFSISRDNGKSTLYLQMDSLRSEDTATYYCTTGSFMYAADYYIMDAWGQGASVTVSS

SEQ ID NO: 167 - protein, 113 aa, synthetic construct
  REGION 1..113: note = 3D6 light chain variable region
DVVMTQTPVSLSVSLGNQASISCRSSQSLLHSDGNTFLSWYFQKPGQSPQLLIYLASNRFSGVSNRFSGSGSGTDFTLKISRVEPEDLGVYYCFQHTHLPLTFGSGTKLEIKR

SEQ ID NO: 168 - protein, 118 aa, synthetic construct
  REGION 1..118: note = 61G6 heavy chain variable region
DVQLQESGPGLVKPSQSLSLTCTVTGYSITSDYAWNWIRQFPGNKLEWMGYISYSGSTRYNPSLKSRISITRDTSKNQFFLQLNSVTSEDTATYYCARQLGLRFFDYWGQGTTLTVSS

SEQ ID NO: 169 - protein, 108 aa, synthetic construct
  REGION 1..108: note = 61G6 light chain variable region
QIVLTQSPALMSASPGEKVTMTCSANSTVNYMYWYQQKPRSSPKPCIYLTSNLASGVPARFSGSGSGTSYSLTISSMEAEDAATYYCQQWNSNPPTFGAGTKLELRRA

SEQ ID NO: 170 - protein, 118 aa, synthetic construct
  REGION 1..118: note = 6H6 heavy chain variable region
QVQLQQSGAELMKPGASVKISCKATGYTFSRYWIEWIKQRPGHGLEWIGEILPGSGSSNYNEKFKDKATFTADTSSNTAYMQFSSLTSEDSAVYYCARKVYYYAMDFWGQGTSVTVSS

SEQ ID NO: 171 - protein, 110 aa, synthetic construct
  REGION 1..110: note = 6H6 light chain variable region
QIVLTQSPAIMSVSLGERVTVTCTASSSVSSSYFHWYQQKPGSSPKLWIYSTSNLASGVPARFSGSGSGTSYSLTISTMEAEDAATYYCHQYHRSPRTFGGGTKLEIKRA

SEQ ID NO: 172 - protein, 119 aa, synthetic construct
  REGION 1..119: note = 61F6 heavy chain variable region
QVQLQQSGAELARPGASVKMSCKASGYTFTSYTMHWVKQRPGQGLEWIGYINPRSVYTNYNQKFKDKATLTADKSSSTAYMQLSSLTSEDSAVYYCARLGGYYDTMDYWGQGTSVTVSS

SEQ ID NO: 173 - protein, 113 aa, synthetic construct
  REGION 1..113: note = 61F6 light chain variable region
DIVVTQSPASLAVSLGQRATISCRASESVDNYGISFMNWFQQKPGQPPKLLIYAASNQGSGVPARFSGSGSGTDFSLNIHPMEEDDTAVYFCQQSKEVPFTFGSGTKLEIKRA

SEQ ID NO: 174 - protein, 122 aa, synthetic construct
  REGION 1..122: note = 1D8 heavy chain variable region
QVTLKESGPGILKPSQTLSLTCSFSGFSLSTSGMGVGWIRQPSGKGLEWLAHIWWDDDKYYSPSLKSQLTISKDTSRNQVFLKITSLDTADTATYYCVRSYYYGSSGAMDYWGQGTSVTVSS

SEQ ID NO: 175 - protein, 118 aa, synthetic construct
  REGION 1..118: note = 1D8 light chain variable region
DIVMTQTPLSLPVSLGDQASISCRSSQSLVHSDGNTYLHWYLQKPGQSPKLLIYKVSKRFSGVPDRFSGSGSGTDFTLKISRVEAEDLGVYFCSQSTHVPPTFGGGTKLEIKRADAAP

SEQ ID NO: 176 - protein, 117 aa, synthetic construct
  REGION 1..117: note = 17F10 heavy chain variable region
EVKLVESGGGFVKPGGSLKLSCAASGFTVRNYAMSWVRQTPEKRLEWVASISTGDRSYLPDSMKGRFTISRDNARNILYLQMSSLRSEDTAIYYCQRYFDFDSFAFWGQGTLVTVSA

SEQ ID NO: 177 - protein, 113 aa, synthetic construct
  REGION 1..113: note = 17F10 light chain variable region
DIQMTQTPSSLSASLGDRVTISCRASQDINNFLNWYQQKPDGSLKLLIYYTSKLHSGVPSRFSGSGSGTDFSLTISNLDQEDVATYFCQQGHTLPPTFGGGTKLEVKRADAAP

SEQ ID NO: 178 - protein, 120 aa, synthetic construct
  REGION 1..120: note = 35D8 heavy chain variable region
EVQLQESGPSLVKPSQTLSLTCSVTGDSITSGYWNWIRKFPGNKLEYMGYISYSGSTYYNPSLRGRISITRDTSKSQYYLQLSSVTTEDTATYYCSRRHLGSGYGWFAYWGQGTLVTVSA

SEQ ID NO: 179 - protein, 114 aa, synthetic construct
  REGION 1..114: note = 35D8 light chain variable region
DIVMTQSHKFMSTSVGDRVSITCKASQDVNTAVAWYQQKPGQSPKLLIYWASTRHTGVPDRFTGSGSGTDYALTINSVQAEDLALYYCQQHSYTPPWTFGGGTKLEIRRADAAP

SEQ ID NO: 180 - protein, 120 aa, synthetic construct
  REGION 1..120: note = 49A1 heavy chain variable region
EVQLQESGPSLVKPSQTLSLTCSVTGDSITSGYWNWIRKFPGNKFEYMGFISYSGNTYYNPSLRSRISITRDTSKNQYFLHLNSVTTEDTATYYCSRRHLISGYGWFAYWGQGTLVTVSA

SEQ ID NO: 181 - protein, 114 aa, synthetic construct
  REGION 1..114: note = 49A1 light chain variable region
VIVMTQSHKFMSTSIGDRVNITCKASQDVISAVAWYQQKPGQSPKLLIYWASTRHTGVPDRFTGSGSGTDFTLTINSVQAEDRALYYCQQHSYTPPWTFGGGTNLEIKRADAAP

SEQ ID NO: 182 - protein, 121 aa, synthetic construct
  REGION 1..121: note = 9E5 heavy chain variable region
QVTLKESGPGILQPSQTLSLTCTFSGFSLSTYGVGVGWIRQPSGKGLEWLANIWWDDDNYYNPSLIHRLTVSKDTSNNQAFLKITNVDTAETATYYCAQIKEPRDWFFEFWGPGTMVSVSS

SEQ ID NO: 183 - protein, 113 aa, synthetic construct
  REGION 1..113: note = 9E5 light chain variable region
DIQMTQTPSSMPASLGERVTIFCRASQGVNNFLTWYQQKPDGTIKPLIFYTSNLQSGVPSRFSGSGSGTDYSLSISSLEPEDFAMYYCQQYHGFPNTFGAGTKLELKRADAAP

SEQ ID NO: 184 - protein, 121 aa, synthetic construct
  REGION 1..121: note = 31H6 heavy chain variable region
QVTLKESGPGILQPSQTLSLTCTFSGFSLSTYGVGVGWIRQPSGKGLEWLANIWWDDDKYYNPSLKNRLTISKDTSNNQAFLKITNVDTAETATYYCAQIKEPRDWFFEFWGPGTMVSVSS

SEQ ID NO: 185 - protein, 113 aa, synthetic construct
  REGION 1..113: note = 31H6 light chain variable region
DIQMTQTPSSMPASLGERVTIFCRASQGVNNYLTWYQQKPDGTIKPLIFYTSNLQSGVPSRFSGSGSGTDYSLSISSLEPEDFAMYYCQQYHGFPNTFGAGTKLELKRADAAP

SEQ ID NO: 186 - protein, 118 aa, synthetic construct
  REGION 1..118: note = humanized 36E5 heavy chain variable region
QVQLVESGGGVVQPGRSLRLSCAASGFTFSSYAMSWVRQAPGKGLEWVASISSGGTTYYPDSVKGRFTISRDNSKNTLYLQMNSLRAEDTAVYYCARVGGYYDSMDYWGQGTLVTVSS

SEQ ID NO: 187 - protein, 112 aa, synthetic construct
  REGION 1..112: note = humanized 36E5 light chain variable region
  SITE 31: note = misc_feature - Xaa can be any naturally occurring amino acid
  SITE 57: note = misc_feature - Xaa can be any naturally occurring amino acid
EIVLTQSPGTLSLSPGERATLSCRASESVDXYGVSFMNWYQQKPGQAPRLLIYAASXQGSGIPDRFSGSGSGTDFTLTISRLEPEDFAVYYCQQTKEVTWTFGQGTKVEIKR

SEQ ID NO: 188 - protein, 123 aa, synthetic construct
  REGION 1..123: note = humanized 3D6 heavy chain variable region
  REGION 97..98: note = misc_feature - Xaa can be any naturally occurring amino acid
QVQLVESGGGVVQPGRSLRLSCAASGFTFSDYYMAWVRQAPGKGLEWVAYIHANGGSTYYRDSVRGRFTISRDNSKNTLYLQMNSLRAEDTAVYYCXXGSFMYAADYYIMDAWGQGTLVTVSS

SEQ ID NO: 189 - protein, 113 aa, synthetic construct
  REGION 1..113: note = humanized 3D6 light chain variable region
DIVMTQSPLSLPVTPGEPASISCRSSQSLLHSDGNTFLSWYLQKPGQSPQLLIYLASNRFSGVPDRFSGSGSGTDFTLKISRVEAEDVGVYYCFQHTHLPLTFGQGTKVEIKR

SEQ ID NO: 190 - protein, 118 aa, synthetic construct
  REGION 1..118: note = humanized 61G6 heavy chain variable region
  SITE 49: note = misc_feature - Xaa can be any naturally occurring amino acid
  SITE 68: note = misc_feature - Xaa can be any naturally occurring amino acid
  SITE 72: note = misc_feature - Xaa can be any naturally occurring amino acid
QVQLQESGPGLVKPSETLSLTCTVSGYSITSDYAWNWIRQPPGKGLEWXGYISYSGSTRYNPSLKSRXTISXDTSKNQFSLKLSSVTAADTAVYYCARQLGLRFFDYWGQGTLVTVSS

SEQ ID NO: 191 - protein, 107 aa, synthetic construct
  REGION 1..107: note = humanized 61G6 light chain variable region
  REGION 45..46: note = misc_feature - Xaa can be any naturally occurring amino acid
EIVLTQSPGTLSLSPGERATLSCSANSTVNYMYWYQQKPGQAPRXXIYLTSNLASGIPDRFSGSGSGTDFTLTISRLEPEDFAVYYCQQWNSNPPTFGQGTKVEIKR

SEQ ID NO: 192 - protein, 118 aa, synthetic construct
  REGION 1..118: note = humanized 6H6 heavy chain variable region
  SITE 48: note = misc_feature - Xaa can be any naturally occurring amino acid
  SITE 68: note = misc_feature - Xaa can be any naturally occurring amino acid
  SITE 70: note = misc_feature - Xaa can be any naturally occurring amino acid
  SITE 72: note = misc_feature - Xaa can be any naturally occurring amino acid
QVQLVQSGAEVKKPGASVKVSCKASGYTFSRYWIEWVRQAPGQGLEWXGEILPGSGSSNYNEKFKDRXTXTXDTSTSTAYMELRSLRSDDTAVYYCARKVYYYAMDFWGQGTLVTVSS

SEQ ID NO: 193 - protein, 109 aa, synthetic construct
  REGION 1..109: note = humanized 6H6 light chain variable region
  SITE 48: note = misc_feature - Xaa can be any naturally occurring amino acid
  SITE 72: note = misc_feature - Xaa can be any naturally occurring amino acid
EIVLTQSPGTLSLSPGERATLSCTASSSVSSSYFHWYQQKPGQAPRLXIYSTSNLASGIPDRFSGSGSGTDXTLTISRLEPEDFAVYYCHQYHRSPRTFGQGTKVEIKR

SEQ ID NO: 194 - protein, 119 aa, synthetic construct
  REGION 1..119: note = humanized 61F6 heavy chain variable region
  SITE 48: note = misc_feature - Xaa can be any naturally occurring amino acid
  SITE 68: note = misc_feature - Xaa can be any naturally occurring amino acid
  SITE 70: note = misc_feature - Xaa can be any naturally occurring amino acid
  SITE 72: note = misc_feature - Xaa can be any naturally occurring amino acid
  SITE 74: note = misc_feature - Xaa can be any naturally occurring amino acid
QVQLVQSGAEVKKPGASVKVSCKASGYTFTSYTMHWVRQAPGQGLEWXGYINPRSVYTNYNQKFKDRXTXTXDXSTSTAYMELRSLRSDDTAVYYCARLGGYYDTMDYWGQGTLVTVSS

SEQ ID NO: 195 - protein, 112 aa, synthetic construct
  REGION 1..112: note = humanized 61F6 light chain variable region
DIQMTQSPSSLSASVGDRVTITCRASESVDNYGISFMNWYQQKPGKAPKLLIYAASNQGSGVPSRFSGSGSGTDFTLTISSLQPEDFATYYCQQSKEVPFTFGQGTKVEIKR

SEQ ID NO: 196 - protein, 122 aa, synthetic construct
  REGION 1..122: note = humanized 1D8 heavy chain variable region
  SITE 24: note = misc_feature - Xaa can be any naturally occurring amino acid
  SITE 69: note = misc_feature - Xaa can be any naturally occurring amino acid
  SITE 73: note = misc_feature - Xaa can be any naturally occurring amino acid
  SITE 75: note = misc_feature - Xaa can be any naturally occurring amino acid
  SITE 80: note = misc_feature - Xaa can be any naturally occurring amino acid
  SITE 98: note = misc_feature - Xaa can be any naturally occurring amino acid
QVQLVESGGGVVQPGRSLRLSCAXSGFSLSTSGMGVGWVRQAPGKGLEWVAHIWWDDDKYYSPSLKSRXTISXDXSKNTXYLQMNSLRAEDTAVYYCXRSYYYGSSGAMDYWGQGTLVTVSS

SEQ ID NO: 197 - protein, 113 aa, synthetic construct
  REGION 1..113: note = humanized 1D8 light chain variable region
DIVMTQSPLSLPVTPGEPASISCRSSQSLVHSDGNTYLHWYLQKPGQSPQLLIYKVSKRFSGVPDRFSGSGSGTDFTLKISRVEAEDVGVYYCSQSTHVPPTFGQGTKVEIKR

SEQ ID NO: 198 - protein, 117 aa, synthetic construct
  REGION 1..117: note = humanized 17F10 heavy chain variable region
  SITE 96: note = misc_feature - Xaa can be any naturally occurring amino acid
QVQLVESGGGVVQPGRSLRLSCAASGFTVRNYAMSWVRQAPGKGLEWVASISTGDRSYLPDSMKGRFTISRDNSKNTLYLQMNSLRAEDTAVYYCXRYFDFDSFAFWGQGTLVTVSS

SEQ ID NO: 199 - protein, 108 aa, synthetic construct
  REGION 1..108: note = humanized 17F10 light chain variable region
DIQMTQSPSSLSASVGDRVTITCRASQDINNFLNWYQQKPGKAPKLLIYYTSKLHSGVPSRFSGSGSGTDFTLTISSLQPEDFATYYCQQGHTLPPTFGQGTKVEIKR

SEQ ID NO: 200 - protein, 120 aa, synthetic construct
  REGION 1..120: note = humanized 35D8 heavy chain variable region
  REGION 47..48: note = misc_feature - Xaa can be any naturally occurring amino acid
  SITE 71: note = misc_feature - Xaa can be any naturally occurring amino acid
  SITE 96: note = misc_feature - Xaa can be any naturally occurring amino acid
QVQLQESGPGLVKPSETLSLTCTVSGDSITSGYWNWIRQPPGKGLEXXGYISYSGSTYYNPSLRGRVTISXDTSKNQFSLKLSSVTAADTAVYYCXRRHLGSGYGWFAYWGQGTLVTVSS

SEQ ID NO: 201 - protein, 109 aa, synthetic construct
  REGION 1..109: note = humanized 35D8 light chain variable region
  SITE 71: note = misc_feature - Xaa can be any naturally occurring amino acid
DIVMTQSPDSLAVSLGERATINCKASQDVNTAVAWYQQKPGQPPKLLIYWASTRHTGVPDRFSGSGSGTDXTLTISSLQAEDVAVYYCQQHSYTPPWTFGQGTKVEIKR

SEQ ID NO: 202 - protein, 120 aa, synthetic construct
  REGION 1..120: note = humanized 49A1 heavy chain variable region
  REGION 47..48: note = misc_feature - Xaa can be any naturally occurring amino acid
  SITE 67: note = misc_feature - Xaa can be any naturally occurring amino acid
  SITE 71: note = misc_feature - Xaa can be any naturally occurring amino acid
  SITE 78: note = misc_feature - Xaa can be any naturally occurring amino acid
  SITE 96: note = misc_feature - Xaa can be any naturally occurring amino acid
QVQLQESGPGLVKPSETLSLTCTVSGDSITSGYWNWIRQPPGKGLEXXGFISYSGNTYYNPSLRSRXTISXDTSKNQXSLKLSSVTAADTAVYYCXRRHLISGYGWFAYWGQGTLVTVSS

SEQ ID NO: 203 - protein, 109 aa, synthetic construct
  REGION 1..109: note = humanized 49A1 light chain variable region
  SITE 1: note = misc_feature - Xaa can be any naturally occurring amino acid
XIVMTQSPDSLAVSLGERATINCKASQDVISAVAWYQQKPGQPPKLLIYWASTRHTGVPDRFSGSGSGTDFTLTISSLQAEDVAVYYCQQHSYTPPWTFGQGTKVEIKR

SEQ ID NO: 204 - protein, 121 aa, synthetic construct
  REGION 1..121: note = humanized 9E5 heavy chain variable region
  SITE 24: note = misc_feature - Xaa can be any naturally occurring amino acid
  REGION 50..51: note = misc_feature - Xaa can be any naturally occurring amino acid
  SITE 69: note = misc_feature - Xaa can be any naturally occurring amino acid
  SITE 71: note = misc_feature - Xaa can be any naturally occurring amino acid
  SITE 73: note = misc_feature - Xaa can be any naturally occurring amino acid
  SITE 80: note = misc_feature - Xaa can be any naturally occurring amino acid
  SITE 99: note = misc_feature - Xaa can be any naturally occurring amino acid
QVQLQESGPGLVKPSETLSLTCTXSGFSLSTYGVGVGWIRQPPGKGLEWXXNIWWDDDNYYNPSLIHRXTXSXDTSKNQXSLKLSSVTAADTAVYYCAXIKEPRDWFFEFWGQGTLVTVSS

SEQ ID NO: 205 - protein, 108 aa, synthetic construct
  REGION 1..108: note = humanized 9E5 light chain variable region
  SITE 46: note = misc_feature - Xaa can be any naturally occurring amino acid
  SITE 49: note = misc_feature - Xaa can be any naturally occurring amino acid
  SITE 71: note = misc_feature - Xaa can be any naturally occurring amino acid
DIQMTQSPSSLSASVGDRVTITCRASQGVNNFLTWYQQKPGKAPKXLIXYTSNLQSGVPSRFSGSGSGTDXTLTISSLQPEDFATYYCQQYHGFPNTFGQGTKVEIKR

SEQ ID NO: 206 - protein, 121 aa, synthetic construct
  REGION 1..121: note = humanized 31H6 heavy chain variable region
  SITE 24: note = misc_feature - Xaa can be any naturally occurring amino acid
  REGION 50..51: note = misc_feature - Xaa can be any naturally occurring amino acid
  SITE 69: note = misc_feature - Xaa can be any naturally occurring amino acid
  SITE 73: note = misc_feature - Xaa can be any naturally occurring amino acid
  SITE 80: note = misc_feature - Xaa can be any naturally occurring amino acid
  SITE 99: note = misc_feature - Xaa can be any naturally occurring amino acid
QVQLQESGPGLVKPSETLSLTCTXSGFSLSTYGVGVGWIRQPPGKGLEWXXNIWWDDDKYYNPSLKNRXTISXDTSKNQXSLKLSSVTAADTAVYYCAXIKEPRDWFFEFWGQGTLVTVSS

SEQ ID NO: 207 - protein, 108 aa, synthetic construct
  REGION 1..108: note = humanized 31H6 light chain variable region
  SITE 46: note = misc_feature - Xaa can be any naturally occurring amino acid
  SITE 49: note = misc_feature - Xaa can be any naturally occurring amino acid
  SITE 71: note = misc_feature - Xaa can be any naturally occurring amino acid
DIQMTQSPSSLSASVGDRVTITCRASQGVNNYLTWYQQKPGKAPKXLIXYTSNLQSGVPSRFSGSGSGTDXTLTISSLQPEDFATYYCQQYHGFPNTFGQGTKVEIKR

SEQ ID NO: 208 - protein, 118 aa, synthetic construct
  REGION 1..118: note = 2155 variable heavy chain
EVKLVESGGGLVKPGGSLKLSCGASGFTISSYAMSWVRQSPEKRLEWVAIISTGGSTYYPDSVRGRFTISRDNARNSLYLQMSSLRSEDTAMYYCARVGGYYDSMDHWGQGTSVTVSS

SEQ ID NO: 209 - protein, 111 aa, synthetic construct
  REGION 1..111: note = 2155 variable light chain
DIVLTQSPASLAVSLGQRATISCRASETVDNYGISFMNWFQQKPGQSPKLLIYAASNQGSGVPARFSGSGSGTDFSLNIHPMEEDDTAMYFCQQSKEVPWTFGGGTKLEIK

SEQ ID NO: 210 - protein, 115 aa, synthetic construct
  REGION 1..115: note = 2155 humanized (HC1) heavy chain
QVTLVESGGGLVKPGGSLTLSCGASGFTISSYAMSWVRQSPGKALEWVAIISTGGSTYYPDSVRGRFTISRDNAKNSLYLTMSSLDSVDTAMYYCARVGGYYDSMDHWGQGTSVT

SEQ ID NO: 211 - protein, 115 aa, synthetic construct
  REGION 1..115: note = 2155 humanized (HC2) heavy chain
QVTLVESGGGLVKPGGSLTLSCGASGFTISSYAMSWVRQSPGKALEWVAIISTGGSTYYPDSVRGRFTISRDNAKNSLYLTMSSLDSVDTATYYCARVGGYYDSMDHWGQGTSVT

SEQ ID NO: 212 - protein, 115 aa, synthetic construct
  REGION 1..115: note = 2155 humanized (HC3a) heavy chain
QVTLVESGGGLVKPGGSLTLSCGASGFTISSYAMSWVRQSPGKALEWVAIISTGGSTYYPDKFRGRFTISRDNAKNSLYLTMSSLRSEDTATYYCARVGGYYDSMDHWGQGTSVT

SEQ ID NO: 213 - protein, 115 aa, synthetic construct
  REGION 1..115: note = humanized (HC3b) heavy chain
QVTLKESGGGLVKPGGSLTLSCGASGFTISSYAMSWVRQSPGKALEWVAIISTGGSTYYPDKFRGRFTISRDNAKNSLYLTMSSLRSEDTATYYCARVGGYYDSMDHWGQGTSVT

SEQ ID NO: 214 - protein, 115 aa, synthetic construct
  REGION 1..115: note = humanized (HC4) heavy chain
EVQLVESGGGLIQPGGSLKLSCAASGFTISSYAMSWVRQAPGKGLEWVAIISTGGSTYYADSVKGRFTISRDNSKNTLYLQMNSLRAEDTAVYYCARVGGYYDSMDHWGQGTSVT

SEQ ID NO: 215 - protein, 109 aa, synthetic construct
  REGION 1..109: note = 2155 humanized (LC1) light chain
DIVLTQSPASLAASVGDRATISCRASETVDNYGISFMNWFQQKPGKSPKLLIYAASNQGSGVPARFSGSGSGTDFSLNIHPMQPDDTATYFCQQSKEVPWTFGGGTKLE

SEQ ID NO: 216 - protein, 109 aa, synthetic construct
  REGION 1..109: note = 2155 humanized (LC2a) light chain
DIVLTQSPASLSASVGDRATISCRASETVDNYGISFMNWFQQKPGQSPKLLIYAASNQGSGVPARFSGSGSGTDFSLTISPMQPDDTATYYCQQSKEVPWTFGGGTKLE

SEQ ID NO: 217 - protein, 109 aa, synthetic construct
  REGION 1..109: note = 2155 humanized (LC2b) light chain
DIVLTQSPASLSASVGDRATISCRASETVDNYGISYMNWFQQKPGQSPKLLIYAASNQGSGVPARFSGSGSGTDFSLTISPMQPDDTATYYCQQSKEVPWTFGGGTKLE

SEQ ID NO: 218 - protein, 109 aa, synthetic construct
  REGION 1..109: note = 2155 humanized (LC3) light chain
DIVLTQSPASLAVSPGQRATITCRASETVDNYGISFMNWFQQKPGQPPKLLIYAASNQGSGVPARFSGSGSGTDFTLTINPVEADDTANYYCQQSKEVPWTFGQGTKVE

SEQ ID NO: 219 - protein, 118 aa, synthetic construct
  REGION 1..118: note = 698 variable heavy chain
EVQLQQSGTVLARPGASVKMSCEASGYSFTTYWMHWIKQRPGQGLEWIGAIYPGNSDTGYNQKFKGKAKLTAVTSATTAYMELSSLTDEDSAVYYCTRTSTYPHFDYWGQGTTLTVSS

SEQ ID NO: 220 - protein, 107 aa, synthetic construct
  REGION 1..107: note = 698 variable light chain
DILLTQSPAILSVSPGERVSFSCRASQSIGTSIHWYQQRTNGSPRLLIKYASESISGIPSRFSGSGSGTDFTLNINSVESEDIADYYCQQSNNWPLTFGAGTKLELK

SEQ ID NO: 221 - protein, 118 aa, synthetic construct
  REGION 1..118: note = 706 variable heavy chain
EVQLQQSGTVLARPGASVKMSCEASGYSFTTYWMHWIKQRPGQGLEWIGAIYPGNSDTGYNQKFKGKAKLTAVTSASTAYMELSSLTNEDSAVYYCTRTSTYPHFDYWGQGTTLTVSS

SEQ ID NO: 222 - protein, 107 aa, synthetic construct
  REGION 1..107: note = 706 variable light chain
DILLTQSPAILSVSPGERVSFSCRASQSIGTSIHWYQQRTNGSPRLLIKYASESISGIPSRFSGSGSGTDFTLNINSVESEDIADYYCQQTNNWPLTFGAGTKLELK

SEQ ID NO: 223 - protein, 118 aa, synthetic construct
  REGION 1..118: note = 827 variable heavy chain
EVQLQQSGTVLARPGASVKMSCETSGYSFTTYWIHWIKQRPGQGLEWIATIYPGNSDAGYNQKFRGKAKLTAVTSASTAYMELSSLTNEDSAVYYCTRSSTYPHFDYWGQGTTLTVSS

SEQ ID NO: 224 - protein, 107 aa, synthetic construct
  REGION 1..107: note = 827 variable light chain
DILLTQSPAILSVSPGERVSFSCRASQSIGTSIHWYQQRTNDSPRLLIKYASESISGIPSRFSGSGSGTDFTLNINSVESEDIADYYCQQTNNWPLTFGAGTKLELK

SEQ ID NO: 225 - protein, 118 aa, synthetic construct
  REGION 1..118: note = 1718 variable heavy chain
QVQVQQSGPELVKPGASVRISCKASDYTFTNYYIHWVRQRPGQGLEWLGWIYPGKGYTNYNEKFKGKATLTADKSSSTAYMQFSSLTSEDSAVYFCASGYGNYYFPYWGQGTLVTVSA

SEQ ID NO: 226 - protein, 106 aa, synthetic construct
  REGION 1..106: note = 1718 variable light chain
IQMTQSSSYLSVSLGGRVTITCKASDHIKNWLAWYQQKPGNVPRLLMSAATSLETGFPSRFSGSGSGKDFTLTITSLQTEDVATYYCQQYWSTPWTFGGGTKLEIK

SEQ ID NO: 227 - protein, 10 aa, synthetic construct
  REGION 1..10: note = 2155 heavy chain CDR3
VGGYYDSMDH

SEQ ID NO: 228 - protein, 9 aa, synthetic construct
  REGION 1..9: note = 2155 heavy chain CDR2
IISTGGSTY

SEQ ID NO: 229 - protein, 10 aa, synthetic construct
  REGION 1..10: note = 2155 heavy chain CDR1
GFTISSYAMS

SEQ ID NO: 230 - protein, 9 aa, synthetic construct
  REGION 1..9: note = 2155 light chain CDR3
QQSKEVPWT

SEQ ID NO: 231 - protein, 7 aa, synthetic construct
  REGION 1..7: note = 2155 light chain CDR2
AASNQGS

SEQ ID NO: 232 - protein, 15 aa, synthetic construct
  REGION 1..15: note = 2155 light chain CDR1
RASETVDNYGISFMN

SEQ ID NO: 233 - protein, 9 aa, synthetic construct
  REGION 1..9: note = 698 and 706 heavy chain CDR3
TSTYPHFDY

SEQ ID NO: 234 - protein, 10 aa, synthetic construct
  REGION 1..10: note = 698 and 706 heavy chain CDR2
AIYPGNSDTG

SEQ ID NO: 235 - protein, 10 aa, synthetic construct
  REGION 1..10: note = 698 and 706 heavy chain CDR1
GYSFTTYWMH

SEQ ID NO: 236 - protein, 9 aa, synthetic construct
  REGION 1..9: note = 698 light chain CDR3
QQSNNWPLT

SEQ ID NO: 237 - protein, 8 aa, synthetic construct
  REGION 1..8: note = 698, 706, 827, and 1649 light chain CDR2
KYASESIS

SEQ ID NO: 238 - protein, 11 aa, synthetic construct
  REGION 1..11: note = 698, 706, 827, and 1649 light chain CDR1
RASQSIGTSIH

SEQ ID NO: 239 - protein, 9 aa, synthetic construct
  REGION 1..9: note = 706, 827, and 1649 light chain CDR3
QQTNNWPLT

SEQ ID NO: 240 - protein, 9 aa, synthetic construct
  REGION 1..9: note = 827 and 1649 heavy chain CDR3
SSTYPHFDY

SEQ ID NO: 241 - protein, 10 aa, synthetic construct
  REGION 1..10: note = 827 heavy chain CDR2
TIYPGNSDAG

SEQ ID NO: 242 - protein, 10 aa, synthetic construct
  REGION 1..10: note = 1649 heavy chain CDR2
AIYPGNSDAG

SEQ ID NO: 243 - protein, 9 aa, synthetic construct
  REGION 1..9: note = 1718 heavy chain CDR3
GYGNYYFPY

SEQ ID NO: 244 - protein, 10 aa, synthetic construct
  REGION 1..10: note = 1718 heavy chain CDR2
WIYPGKGYTN

SEQ ID NO: 245 - protein, 9 aa, synthetic construct
  REGION 1..9: note = 1718 heavy chain CDR1
DYTFTNYYI

SEQ ID NO: 246 - protein, 9 aa, synthetic construct
  REGION 1..9: note = 1718 light chain CDR3
QQTWSTPWT

SEQ ID NO: 247 - protein, 7 aa, synthetic construct
  REGION 1..7: note = 1718 light chain CDR2
AATSLET

SEQ ID NO: 248 - protein, 11 aa, synthetic construct
  REGION 1..11: note = 1718 light chain CDR1
KASDHIKNWLA

SEQ ID NO: 249 - protein, 10 aa, synthetic construct
  REGION 1..10: note = 827 and 1649 heavy chain CDR1
GYSFTTYWIH

SEQ ID NO: 250 - protein, 453 aa, synthetic construct
  REGION 1..453: note = 1D7 heavy chain
QVQLVESGGGVVQPGRSLRLSCAASGFTFSSYGMHWVRQAPGKGLEWVTVIWYEGSNKYYADSVKGRFTISRDNSKNTLYLQMNSLRAEDTAVYYCARGGQLGKYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 251 - protein, 214 aa, synthetic construct
  REGION 1..214: note = 1D7 light chain
DIQMTQSPSSLSASVGDRVTITCRASQGIRNDLGWYQQKPGKAPKRLIYDASSLQSGVPSRFSGSGSGTEFTLTISSLQPEDFATYYCLQHNNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 252 - protein, 123 aa, synthetic construct
  REGION 1..123: note = 1D7 variable heavy chain
QVQLVESGGGVVQPGRSLRLSCAASGFTFSSYGMHWVRQAPGKGLEWVTVIWYEGSNKYYADSVKGRFTISRDNSKNTLYLQMNSLRAEDTAVYYCARGGQLGKYYYYGMDVWGQGTTVTVSS

SEQ ID NO: 253 - protein, 108 aa, synthetic construct
  REGION 1..108: note = 1D7 variable light chain
DIQMTQSPSSLSASVGDRVTITCRASQGIRNDLGWYQQKPGKAPKRLIYDASSLQSGVPSRFSGSGSGTEFTLTISSLQPEDFATYYCLQHNNYPWTFGQGTKVEIKR

SEQ ID NO: 254 - protein, 5 aa, synthetic construct
  REGION 1..5: note = 1D7 heavy chain CDR1
SYGMH

SEQ ID NO: 255 - protein, 17 aa, synthetic construct
  REGION 1..17: note = 1D7 heavy chain CDR2
VIWYEGSNKYYADSVKG

SEQ ID NO: 256 - protein, 14 aa, synthetic construct
  REGION 1..14: note = 1D7 heavy chain CDR3
GGQLGKYYYYGMDV

SEQ ID NO: 257 - protein, 11 aa, synthetic construct
  REGION 1..11: note = 1D7 light chain CDR1
RASQGIRNDLG

SEQ ID NO: 258 - protein, 7 aa, synthetic construct
  REGION 1..7: note = 1D7 light chain CDR2
DASSLQS

SEQ ID NO: 259 - protein, 9 aa, synthetic construct
  REGION 1..9: note = 1D7 light chain CDR3
LQHNNYPWT

SEQ ID NO: 260 - protein, 453 aa, synthetic construct
  REGION 1..453: note = 33C9 heavy chain
QVQVVESGGGVVQPGRSLRLSCAASGFTFSSYGMHWVRQAPGKGLEWVSVIWYEGSNKYYADSVKGRFTISRDNSKNTLYLQMNSLRAEDTAVYYCARGGLLGYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 261 - protein, 214 aa, synthetic construct
  REGION 1..214: note = 33C9 light chain
DIQMTQSPSSLSASVGDRVTITCRASQGIRNDLGWYQQKPGKAPKRLIYDASSLQSGVPSRFSGSGSGTEFTLTISSLQPEDFATYYCLQHHSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 262 - protein, 123 aa, synthetic construct
  REGION 1..123: note = 33C9 variable heavy chain
QVQVVESGGGVVQPGRSLRLSCAASGFTFSSYGMHWVRQAPGKGLEWVSVIWYEGSNKYYADSVKGRFTISRDNSKNTLYLQMNSLRAEDTAVYYCARGGLLGYYYYYGMDVWGQGTTVTVSS

SEQ ID NO: 263 - protein, 108 aa, synthetic construct
  REGION 1..108: note = 33C9 variable light chain
DIQMTQSPSSLSASVGDRVTITCRASQGIRNDLGWYQQKPGKAPKRLIYDASSLQSGVPSRFSGSGSGTEFTLTISSLQPEDFATYYCLQHHSYPWTFGQGTKVEIKR

SEQ ID NO: 264 - protein, 5 aa, synthetic construct
  REGION 1..5: note = 33C9 heavy chain CDR1
SYGMH

SEQ ID NO: 265 - protein, 17 aa, synthetic construct
  REGION 1..17: note = 33C9 heavy chain CDR2
VIWYEGSNKYYADSVKG

SEQ ID NO: 266 - protein, 14 aa, synthetic construct
  REGION 1..14: note = 33C9 heavy chain CDR3
GGLLGYYYYYGMDV

SEQ ID NO: 267 - protein, 11 aa, synthetic construct
  REGION 1..11: note = 33C9 light chain CDR1
RASQGIRNDLG

SEQ ID NO: 268 - protein, 7 aa, synthetic construct
  REGION 1..7: note = 33C9 light chain CDR2
DASSLQS

SEQ ID NO: 269 - protein, 9 aa, synthetic construct
  REGION 1..9: note = 33C9 light chain CDR3
LQHHSYPWT

SEQ ID NO: 270 - protein, 453 aa, synthetic construct
  REGION 1..453: note = 33F6 heavy chain
QVQLVESGGGVVQPGRSLRLSCAASGFTFSNYGMHWVRQAPGKGLEWVAVIWYVGSNKYYADSVKGRFTISRDNSKNTLYLQMNSLRAEDTAVYYCARGGELRLYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 271 - protein, 214 aa, synthetic construct
  REGION 1..214: note = 33F6 light chain
DIQMTQSPSSLSASVGDRVTITCRASQGIRNDLGWYQQKPGKAPKRLIYAASSLQSGVPSRFSGSGSGTEFTLTVSSLQPEDFATYYCLQLNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 272 - protein, 123 aa, synthetic construct
  REGION 1..123: note = 33F6 variable heavy chain
QVQLVESGGGVVQPGRSLRLSCAASGFTFSNYGMHWVRQAPGKGLEWVAVIWYVGSNKYYADSVKGRFTISRDNSKNTLYLQMNSLRAEDTAVYYCARGGELRLYYYYGMDVWGQGTTVTVSS

SEQ ID NO: 273 - protein, 108 aa, synthetic construct
  REGION 1..108: note = 33F6 variable light chain
DIQMTQSPSSLSASVGDRVTITCRASQGIRNDLGWYQQKPGKAPKRLIYAASSLQSGVPSRFSGSGSGTEFTLTVSSLQPEDFATYYCLQLNSYPWTFGQGTKVEIKR

SEQ ID NO: 274 - protein, 5 aa, synthetic construct
  REGION 1..5: note = 33F6 heavy chain CDR1
NYGMH

SEQ ID NO: 275 - protein, 17 aa, synthetic construct
  REGION 1..17: note = 33F6 heavy chain CDR2
VIWYVGSNKYYADSVKG

SEQ ID NO: 276 - protein, 14 aa, synthetic construct
  REGION 1..14: note = 33F6 heavy chain CDR3
GGELRLYYYYGMDV

SEQ ID NO: 277 - protein, 11 aa, synthetic construct
  REGION 1..11: note = 33F6 light chain CDR1
RASQGIRNDLG

SEQ ID NO: 278 - protein, 7 aa, synthetic construct
  REGION 1..7: note = 33F6 light chain CDR2
AASSLQS

SEQ ID NO: 279 - protein, 9 aa, synthetic construct
  REGION 1..9: note = 33F6 light chain CDR3
LQLNSYPWT

SEQ ID NO: 280 - protein, 453 aa, synthetic construct
  REGION 1..453: note = 34G4 heavy chain
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGQLGYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 281 - protein, 214 aa, synthetic construct
  REGION 1..214: note = 34G4 light chain
DIQMTQSPSSLSASVGDRVTITCRASQGIRNDLGWYQQKPGKAPKRLIYDASSLQSGVPSRFSGSGSGTDFTLTISSLQPEDFATYYCLQLNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 282 - protein, 123 aa, synthetic construct
  REGION 1..123: note = 34G4 variable heavy chain
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGQLGYYYYYGMDVWGQGTTVTVSS

SEQ ID NO: 283 - protein, 108 aa, synthetic construct
  REGION 1..108: note = 34G4 variable light chain
DIQMTQSPSSLSASVGDRVTITCRASQGIRNDLGWYQQKPGKAPKRLIYDASSLQSGVPSRFSGSGSGTDFTLTISSLQPEDFATYYCLQLNSYPWTFGQGTKVEIKR

SEQ ID NO: 284 - protein, 5 aa, synthetic construct
  REGION 1..5: note = 34G4 heavy chain CDR1
SYGMH

SEQ ID NO: 285 - protein, 17 aa, synthetic construct
  REGION 1..17: note = 34G4 heavy chain CDR2
VIWYEGSNKYYADSVKG

SEQ ID NO: 286 - protein, 14 aa, synthetic construct
  REGION 1..14: note = 34G4 heavy chain CDR3
GGQLGYYYYYGMDV

SEQ ID NO: 287 - protein, 11 aa, synthetic construct
  REGION 1..11: note = 34G4 light chain CDR1
RASQGIRNDLG

SEQ ID NO: 288 - protein, 7 aa, synthetic construct
  REGION 1..7: note = 34G4 light chain CDR2
DASSLQS

SEQ ID NO: 289 - protein, 9 aa, synthetic construct
  REGION 1..9: note = 34G4 light chain CDR3
LQLNSYPWT

SEQ ID NO: 290 - protein, 453 aa, synthetic construct
  REGION 1..453: note = 35B10 heavy chain
QVQLVESGGGVVQPGRSLRLSCAASGFTFSSYGMHWVRQAPGKGLEWVAVIWYAGSNKYYADSVKGRFTISRDNSKNTLYLQMNSLRAEDTAVYYCARGGELSFYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 291 - protein, 214 aa, synthetic construct
  REGION 1..214: note = 35B10 light chain
DIQMTQSPSSLSASVGDRVTITCRASQGIRNDLGWYQQKPGKAPKRLIYAASTLQSGVPSRFSGSGSGTEFTLTISSLQPEDFATYYCLQHNNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 292 - protein, 123 aa, synthetic construct
  REGION 1..123: note = 35B10 variable heavy chain
QVQLVESGGGVVQPGRSLRLSCAASGFTFSSYGMHWVRQAPGKGLEWVAVIWYAGSNKYYADSVKGRFTISRDNSKNTLYLQMNSLRAEDTAVYYCARGGELSFYYYYGMDVWGQGTTVTVSS

SEQ ID NO: 293 - protein, 108 aa, synthetic construct
  REGION 1..108: note = 35B10 variable light chain
DIQMTQSPSSLSASVGDRVTITCRASQGIRNDLGWYQQKPGKAPKRLIYAASTLQSGVPSRFSGSGSGTEFTLTISSLQPEDFATYYCLQHNNYPWTFGQGTKVEIKR

SEQ ID NO: 294 - protein, 5 aa, synthetic construct
  REGION 1..5: note = 35B10 heavy chain CDR1
SYGMH

SEQ ID NO: 295 - protein, 17 aa, synthetic construct
  REGION 1..17: note = 35B10 heavy chain CDR2
VIWYAGSNKYYADSVKG

SEQ ID NO: 296 - protein, 14 aa, synthetic construct
  REGION 1..14: note = 35B10 heavy chain CDR3
GGELSFYYYYGMDV

SEQ ID NO: 297 - protein, 11 aa, synthetic construct
  REGION 1..11: note = 35B10 light chain CDR1
RASQGIRNDLG

SEQ ID NO: 298 - protein, 7 aa, synthetic construct
  REGION 1..7: note = 35B10 light chain CDR2
AASTLQS

SEQ ID NO: 299 - protein, 9 aa, synthetic construct
  REGION 1..9: note = 35B10 light chain CDR3
LQHNNYPWT

SEQ ID NO: 300 - protein, 453 aa, synthetic construct
  REGION 1..453: note = 41E11 heavy chain
QVQVVESGGGVVQPGRSLRLSCAASGFTFSSYGMYWVRQAPGKGLEWVAVIWYEGSNKYYADSVRGRFTISRDNSKNTLYLQMNSLRAEDTALYYCARGGQLGKDYYSGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 301 - protein, 214 aa, synthetic construct
  REGION 1..214: note = 41E11 light chain
DIQMTQSPSSLSASVGDRVTITCRASQVIRNDLGWYQQKPGKAPKRLIYAASSLQSGVPSRFSGSGSGTEFTLTISSLQPEDFATYYCLQHNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 302 - protein, 123 aa, synthetic construct
  REGION 1..123: note = 41E11 variable heavy chain
QVQVVESGGGVVQPGRSLRLSCAASGFTFSSYGMYWVRQAPGKGLEWVAVIWYEGSNKYYADSVRGRFTISRDNSKNTLYLQMNSLRAEDTALYYCARGGQLGKDYYSGMDVWGQGTTVTVSS

SEQ ID NO: 303 - protein, 108 aa, synthetic construct
  REGION 1..108: note = 41E11 variable light chain
DIQMTQSPSSLSASVGDRVTITCRASQVIRNDLGWYQQKPGKAPKRLIYAASSLQSGVPSRFSGSGSGTEFTLTISSLQPEDFATYYCLQHNSYPLTFGGGTKVEIKR

SEQ ID NO: 304 - protein, 5 aa, synthetic construct
  REGION 1..5: note = 41E11 heavy chain CDR1
SYGMY

SEQ ID NO: 305 - protein, 17 aa, synthetic construct
  REGION 1..17: note = 41E11 heavy chain CDR2
VIWYEGSNKYYADSVRG

SEQ ID NO: 306 - protein, 14 aa, synthetic construct
  REGION 1..14: note = 41E11 heavy chain CDR3
GGQLGKDYYSGMDV

SEQ ID NO: 307 - protein, 11 aa, synthetic construct
  REGION 1..11: note = 41E11 light chain CDR1
RASQVIRNDLG

SEQ ID NO: 308 - protein, 7 aa, synthetic construct
  REGION 1..7: note = 41E11 light chain CDR2
AASSLQS

SEQ ID NO: 309 - protein, 9 aa, synthetic construct
  REGION 1..9: note = 41E11 light chain CDR3
LQHNSYPLT

SEQ ID NO: 310 - protein, 453 aa, synthetic construct
  REGION 1..453: note = 41G5 heavy chain
QVQLVESGGGVVQPGRSLRLSCAASGFTFSSYGMHWVRQAPGKGLEWVAVIWYPGSNKYYADSVKGRFTISRDNSKNTLYLQMNSLRAEDTAVYYCARGGELGRYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 311 - protein, 214 aa, synthetic construct
  REGION 1..214: note = 41G5 light chain
DIQMTQSPSSLSASVGDRVTVTCRASQGIRNDLGWYQQKPGKAPKRLIYAASSLQSGVPSRFSGSGSGTEFTLTISSLQPEDFATYYCLQHNNYPWTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 312 - protein, 123 aa, synthetic construct
  REGION 1..123: note = 41G5 variable heavy chain
QVQLVESGGGVVQPGRSLRLSCAASGFTFSSYGMHWVRQAPGKGLEWVAVIWYPGSNKYYADSVKGRFTISRDNSKNTLYLQMNSLRAEDTAVYYCARGGELGRYYYYGMDVWGQGTTVTVSS

SEQ ID NO: 313 - protein, 108 aa, synthetic construct
  REGION 1..108: note = 41G5 variable light chain
DIQMTQSPSSLSASVGDRVTVTCRASQGIRNDLGWYQQKPGKAPKRLIYAASSLQSGVPSRFSGSGSGTEFTLTISSLQPEDFATYYCLQHNNYPWTFGQGTKVDIKR

SEQ ID NO: 314 - protein, 5 aa, synthetic construct
  REGION 1..5: note = 41G5 heavy chain CDR1
SYGMH

SEQ ID NO: 315 - protein, 17 aa, synthetic construct
  REGION 1..17: note = 41G5 heavy chain CDR2
VIWYPGSNKYYADSVKG

SEQ ID NO: 316 - protein, 14 aa, synthetic construct
  REGION 1..14: note = 41G5 heavy chain CDR3
GGELGRYYYYGMDV

SEQ ID NO: 317 - protein, 11 aa, synthetic construct
  REGION 1..11: note = 41G5 light chain CDR1
RASQGIRNDLG

SEQ ID NO: 318 - protein, 7 aa, synthetic construct
  REGION 1..7: note = 41G5 light chain CDR2
AASSLQS

SEQ ID NO: 319 - protein, 9 aa, synthetic construct
  REGION 1..9: note = 41G5 light chain CDR3
LQHNNYPWT

SEQ ID NO: 320 - protein, 453 aa, synthetic construct
  REGION 1..453: note = 42A11 heavy chain
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGQLGYYYYSGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 321 - protein, 214 aa, synthetic construct
  REGION 1..214: note = 42A11 light chain
DIQMTQSPSSLSASVGDRVTITCRASQGIRNDLGWYQQKPGKAPKRLIYDASSLQSGVPSRFSGSGSGTDFTLTISSLQPEEFATYYCLQHNNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 322 - protein, 123 aa, synthetic construct
  REGION 1..123: note = 42A11 variable heavy chain
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGQLGYYYYSGMDVWGQGTTVTVSS

SEQ ID NO: 323 - protein, 108 aa, synthetic construct
  REGION 1..108: note = 42A11 variable light chain
DIQMTQSPSSLSASVGDRVTITCRASQGIRNDLGWYQQKPGKAPKRLIYDASSLQSGVPSRFSGSGSGTDFTLTISSLQPEEFATYYCLQHNNYPWTFGQGTKVEIKR

SEQ ID NO: 324 - protein, 5 aa, synthetic construct
  REGION 1..5: note = 42A11 heavy chain CDR1
SYGMH

SEQ ID NO: 325 - protein, 17 aa, synthetic construct
  REGION 1..17: note = 42A11 heavy chain CDR2
VIWYEGSNKYYADSVKG

SEQ ID NO: 326 - protein, 14 aa, synthetic construct
  REGION 1..14: note = 42A11 heavy chain CDR3
GGQLGYYYYSGMDV

SEQ ID NO: 327 - protein, 11 aa, synthetic construct
  REGION 1..11: note = 42A11 light chain CDR1
RASQGIRNDLG

SEQ ID NO: 328 - protein, 7 aa, synthetic construct
  REGION 1..7: note = 42A11 light chain CDR2
DASSLQS

SEQ ID NO: 329 - protein, 9 aa, synthetic construct
  REGION 1..9: note = 42A11 light chain CDR3
LQHNNYPWT

SEQ ID NO: 330 - protein, 450 aa, synthetic construct
  REGION 1..450: note = 44C1 heavy chain
QVQLVESGGGVVQPGRSLRLSCAASGFTLSSYGMHWVRQAPGKGLEWVAVIWYDGSNKYYADSVKGRFTISRDNSKNTLYLQMNSLRAEDTAVYYCARRGTVTTPDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 331 - protein, 216 aa, synthetic construct
  REGION 1..216: note = 44C1 light chain
QSALTQPASVSGSPGQSITISCTGTSSDVGTYNLVSWYQQHPGKAPKLMIYEVSKRPSGVSNRFSGSKSGNTASLTISGLQAEDEADYYCCSYAGFSTWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 332 - protein, 120 aa, synthetic construct
  REGION 1..120: note = 44C1 variable heavy chain
QVQLVESGGGVVQPGRSLRLSCAASGFTLSSYGMHWVRQAPGKGLEWVAVIWYDGSNKYYADSVKGRFTISRDNSKNTLYLQMNSLRAEDTAVYYCARRGTVTTPDFDYWGQGTLVTVSS

SEQ ID NO: 333 - protein, 111 aa, synthetic construct
  REGION 1..111: note = 44C1 variable light chain
QSALTQPASVSGSPGQSITISCTGTSSDVGTYNLVSWYQQHPGKAPKLMIYEVSKRPSGVSNRFSGSKSGNTASLTISGLQAEDEADYYCCSYAGFSTWVFGGGTKLTVLG

SEQ ID NO: 334 - protein, 5 aa, synthetic construct
  REGION 1..5: note = 44C1 heavy chain CDR1
SYGMH

SEQ ID NO: 335 - protein, 17 aa, synthetic construct
  REGION 1..17: note = 44C1 heavy chain CDR2
VIWYDGSNKYYADSVKG

SEQ ID NO: 336 - protein, 11 aa, synthetic construct
  REGION 1..11: note = 44C1 heavy chain CDR3
RGTVTTPDFDY

SEQ ID NO: 337 - protein, 14 aa, synthetic construct
  REGION 1..14: note = 44C1 light chain CDR1
TGTSSDVGTYNLVS

SEQ ID NO: 338 - protein, 7 aa, synthetic construct
  REGION 1..7: note = 44C1 light chain CDR2
EVSKRPS

SEQ ID NO: 339 - protein, 10 aa, synthetic construct
  REGION 1..10: note = 44C1 light chain CDR3
CSYAGFSTWV

SEQ ID NO: 340 - protein, 447 aa, synthetic construct
  REGION 1..447: note = 45A8 heavy chain
QVQLVESGGGVVQPGRSLRLSCAASGFTFSSYGMHWVRQAPGKGLEWVAVIWHDGSNKYYADSVKGRFTISKDNSKNTLYLQMNSLRAEDTAVYYCAREYGGNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 341 - protein, 216 aa, synthetic construct
  REGION 1..216: note = 45A8 light chain
QSALTQPASVSGSPGQSITISCTGTSSDVGTYNLVSWYQQHPGKAPKLMIYEVSKRPSGISNRFSGSKSGNTASLTISGLQAEDEADYYCCSYAGYSTWVFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 342 - protein, 117 aa, synthetic construct
  REGION 1..117: note = 45A8 variable heavy chain
QVQLVESGGGVVQPGRSLRLSCAASGFTFSSYGMHWVRQAPGKGLEWVAVIWHDGSNKYYADSVKGRFTISKDNSKNTLYLQMNSLRAEDTAVYYCAREYGGNFDYWGQGTLVTVSS

SEQ ID NO: 343 - protein, 111 aa, synthetic construct
  REGION 1..111: note = 45A8 variable light chain
QSALTQPASVSGSPGQSITISCTGTSSDVGTYNLVSWYQQHPGKAPKLMIYEVSKRPSGISNRFSGSKSGNTASLTISGLQAEDEADYYCCSYAGYSTWVFGGGTKLTVLR

SEQ ID NO: 344 - protein, 5 aa, synthetic construct
  REGION 1..5: note = 45A8 heavy chain CDR1
SYGMH

SEQ ID NO: 345 - protein, 17 aa, synthetic construct
  REGION 1..17: note = 45A8 heavy chain CDR2
VIWHDGSNKYYADSVKG

SEQ ID NO: 346 - protein, 8 aa, synthetic construct
  REGION 1..8: note = 45A8 heavy chain CDR3
EYGGNFDY

SEQ ID NO: 347 - protein, 14 aa, synthetic construct
  REGION 1..14: note = 45A8 light chain CDR1
TGTSSDVGTYNLVS

SEQ ID NO: 348 - protein, 7 aa, synthetic construct
  REGION 1..7: note = 45A8 light chain CDR2
EVSKRPS

SEQ ID NO: 349 - protein, 10 aa, synthetic construct
  REGION 1..10: note = 45A8 light chain CDR3
CSYAGYSTWV

SEQ ID NO: 350 - protein, 454 aa, synthetic construct
  REGION 1..454: note = 46E11 heavy chain
QVQLVESGGGVVQPGRSLRLSCAASGFTFSSYGMHWVRQAPGKGLEWVAVIWYAGSNKYYADSVKGRFTISRDNSKNTLYLQMNSLRAEDTAVYYCARGDILTGYSLYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 351 - protein, 214 aa, synthetic construct
  REGION 1..214: note = 46E11 light chain
DIQMTQSPSSLSASVGDRVTITCRASQGIRNDLGWYQQKPGKAPKRLIYAASSLQSGVPSRFSGSGSGAEFTLTISSLQPEDFATYYCLQHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 352 - protein, 124 aa, synthetic construct
  REGION 1..124: note = 46E11 variable heavy chain
QVQLVESGGGVVQPGRSLRLSCAASGFTFSSYGMHWVRQAPGKGLEWVAVIWYAGSNKYYADSVKGRFTISRDNSKNTLYLQMNSLRAEDTAVYYCARGDILTGYSLYYGMDVWGQGTTVTVSS

SEQ ID NO: 353 - protein, 108 aa, synthetic construct
  REGION 1..108: note = 46E11 variable light chain
DIQMTQSPSSLSASVGDRVTITCRASQGIRNDLGWYQQKPGKAPKRLIYAASSLQSGVPSRFSGSGSGAEFTLTISSLQPEDFATYYCLQHNSYPWTFGQGTKVEIKR

SEQ ID NO: 354 - protein, 5 aa, synthetic construct
  REGION 1..5: note = 46E11 heavy chain CDR1
SYGMH

SEQ ID NO: 355 - protein, 17 aa, synthetic construct
  REGION 1..17: note = 46E11 heavy chain CDR2
VIWYAGSNKYYADSVKG

SEQ ID NO: 356 - protein, 15 aa, synthetic construct
  REGION 1..15: note = 46E11 heavy chain CDR3
GDILTGYSLYYGMDV

SEQ ID NO: 357 - protein, 11 aa, synthetic construct
  REGION 1..11: note = 46E11 light chain CDR1
RASQGIRNDLG

SEQ ID NO: 358 - protein, 7 aa, synthetic construct
  REGION 1..7: note = 46E11 light chain CDR2
AASSLQS

SEQ ID NO: 359 - protein, 9 aa, synthetic construct
  REGION 1..9: note = 46E11 light chain CDR3
LQHNSYPWT

SEQ ID NO: 360 - protein, 453 aa, synthetic construct
  REGION 1..453: note = 48H12 heavy chain
QVQLVESGGGVVQPGRSLRLSCAASGFTFSSYGMHWVRQAPGKGLEWVAVIWYAGSNKYYADSVKGRFTISRDNSKNTVYLQMNSLRAEDTAVYYCARGGQLALYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 361 - protein, 214 aa, synthetic construct
  REGION 1..214: note = 48H12 light chain
DIQMTQSPSSLSASVGDRVTITCRASQGIRNDLGWYQQKPGKAPKRLIYAASSLQSGVPSRFSGSGSGTEFTLTISSLQPEDFATYYCLQHNNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 362 - protein, 123 aa, synthetic construct
  REGION 1..123: note = 48H12 variable heavy chain
QVQLVESGGGVVQPGRSLRLSCAASGFTFSSYGMHWVRQAPGKGLEWVAVIWYAGSNKYYADSVKGRFTISRDNSKNTVYLQMNSLRAEDTAVYYCARGGQLALYYYYGMDVWGQGTTVTVSS

SEQ ID NO: 363 - protein, 108 aa, synthetic construct
  REGION 1..108: note = 48H12 variable light chain
DIQMTQSPSSLSASVGDRVTITCRASQGIRNDLGWYQQKPGKAPKRLIYAASSLQSGVPSRFSGSGSGTEFTLTISSLQPEDFATYYCLQHNNYPWTFGQGTKVEIKR

SEQ ID NO: 364 - protein, 5 aa, synthetic construct
  REGION 1..5: note = 48H12 heavy chain CDR1
SYGMH

SEQ ID NO: 365 - protein, 17 aa, synthetic construct
  REGION 1..17: note = 48H12 heavy chain CDR2
VIWYAGSNKYYADSVKG

SEQ ID NO: 366 - protein, 14 aa, synthetic construct
  REGION 1..14: note = 48H12 heavy chain CDR3
GGQLALYYYYGMDV

SEQ ID NO: 367 - protein, 11 aa, synthetic construct
  REGION 1..11: note = 48H12 light chain CDR1
RASQGIRNDLG

SEQ ID NO: 368 - protein, 7 aa, synthetic construct
  REGION 1..7: note = 48H12 light chain CDR2
AASSLQS

SEQ ID NO: 369 - protein, 9 aa, synthetic construct
  REGION 1..9: note = 48H12 light chain CDR3
LQHNNYPWT

SEQ ID NO: 370 - protein, 453 aa, synthetic construct
  REGION 1..453: note = 48H7 heavy chain
QVQLVESGGGVVQPGRSLRLSCAASGFTFSSYGMYWVRQAPGKGLEWVAVIWYEGSNKYYADSVKGRFTISRDNSKNTLYLQMNSLRAEDTAVYFCARGGELGRDYYSGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 371 - protein, 214 aa, synthetic construct
  REGION 1..214: note = 48H7 light chain
DIQMTQSPSSLSASVGDRVTITCRASQVIRNDLGWYQQKPGKAPKRLIYAASSLQSGVPSRFSGSGSGTEFTLTISSLQPEDFATYYCLQHNSYPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 372 - protein, 123 aa, synthetic construct
  REGION 1..123: note = 48H7 variable heavy chain
QVQLVESGGGVVQPGRSLRLSCAASGFTFSSYGMYWVRQAPGKGLEWVAVIWYEGSNKYYADSVKGRFTISRDNSKNTLYLQMNSLRAEDTAVYFCARGGELGRDYYSGMDVWGQGTTVTVSS

SEQ ID NO: 373 - protein, 108 aa, synthetic construct
  REGION 1..108: note = 48H7 variable light chain
DIQMTQSPSSLSASVGDRVTITCRASQVIRNDLGWYQQKPGKAPKRLIYAASSLQSGVPSRFSGSGSGTEFTLTISSLQPEDFATYYCLQHNSYPITFGGGTKVEIKR

SEQ ID NO: 374 - protein, 5 aa, synthetic construct
  REGION 1..5: note = 48H7 heavy chain CDR1
SYGMY

SEQ ID NO: 375 - protein, 17 aa, synthetic construct
  REGION 1..17: note = 48H7 heavy chain CDR2
VIWYEGSNKYYADSVKG

SEQ ID NO: 376 - protein, 14 aa, synthetic construct
  REGION 1..14: note = 48H7 heavy chain CDR3
GGELGRDYYSGMDV

SEQ ID NO: 377 - protein, 11 aa, synthetic construct
  REGION 1..11: note = 48H7 light chain CDR1
RASQVIRNDLG

SEQ ID NO: 378 - protein, 7 aa, synthetic construct
  REGION 1..7: note = 48H7 light chain CDR2
AASSLQS

SEQ ID NO: 379 - protein, 9 aa, synthetic construct
  REGION 1..9: note = 48H7 light chain CDR3
LQHNSYPIT

SEQ ID NO: 380 - protein, 453 aa, synthetic construct
  REGION 1..453: note = 49D9 heavy chain
QMQLVESGGGVVQPGRSLRLSCAASGFTFSSYGMHWVRQAPGKGLEWVAVIWYAGSNKYYADSVKGRFTISRDNSKNTLYLQMNSLRAEDTAVYYCARGGRLGFYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 381 - protein, 214 aa, synthetic construct
  REGION 1..214: note = 49D9 light chain
DIQMTQSPSSLSASVGDRVTITCRASQGIRNDLGWYQQKPGKAPKRLIYAASSLQSGVPSRFSGSGSGTEFTLTISSLQPEDFATYYCLQLNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 382 - protein, 123 aa, synthetic construct
  REGION 1..123: note = 49D9 variable heavy chain
QMQLVESGGGVVQPGRSLRLSCAASGFTFSSYGMHWVRQAPGKGLEWVAVIWYAGSNKYYADSVKGRFTISRDNSKNTLYLQMNSLRAEDTAVYYCARGGRLGFYYYYGMDVWGQGTTVTVSS

SEQ ID NO: 383 - protein, 108 aa, synthetic construct
  REGION 1..108: note = 49D9 variable light chain
DIQMTQSPSSLSASVGDRVTITCRASQGIRNDLGWYQQKPGKAPKRLIYAASSLQSGVPSRFSGSGSGTEFTLTISSLQPEDFATYYCLQLNSYPWTFGQGTKVEIKR

SEQ ID NO: 384 - protein, 5 aa, synthetic construct
  REGION 1..5: note = 49D9 heavy chain CDR1
SYGMH

SEQ ID NO: 385 - protein, 17 aa, synthetic construct
  REGION 1..17: note = 49D9 heavy chain CDR2
VIWYAGSNKYYADSVKG

SEQ ID NO: 386 - protein, 14 aa, synthetic construct
  REGION 1..14: note = 49D9 heavy chain CDR3
GGRLGFYYYYGMDV

SEQ ID NO: 387 - protein, 11 aa, synthetic construct
  REGION 1..11: note = 49D9 light chain CDR1
RASQGIRNDLG

SEQ ID NO: 388 - protein, 7 aa, synthetic construct
  REGION 1..7: note = 49D9 light chain CDR2
AASSLQS

SEQ ID NO: 389 - protein, 9 aa, synthetic construct
  REGION 1..9: note = 49D9 light chain CDR3
LQLNSYPWT

SEQ ID NO: 390 - protein, 447 aa, synthetic construct
  REGION 1..447: note = 49E2 heavy chain
QVQLVESGGGVVQPGRSLRLSCAASGFTFSSYGMHWVRQAPGKGLEWVAVIWSDGNNKYYEDSVKGRFTISRDSSKNTLFLQMNSLRAEDTAVYYCARDTATPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 391 - protein, 216 aa, synthetic construct
  REGION 1..216: note = 49E2 light chain
QSALTQPASVSGSPGQSITISCTGTSSDVGIYNLVSWYQQHPGKAPKLMIHEVSKRPSGVSNRFSGSKSGNTASLTISGLQAEDEADYYCCSYAGISTWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 392 - protein, 117 aa, synthetic construct
  REGION 1..117: note = 49E2 variable heavy chain
QVQLVESGGGVVQPGRSLRLSCAASGFTFSSYGMHWVRQAPGKGLEWVAVIWSDGNNKYYEDSVKGRFTISRDSSKNTLFLQMNSLRAEDTAVYYCARDTATPFDYWGQGTLVTVSS

SEQ ID NO: 393 - protein, 111 aa, synthetic construct
  REGION 1..111: note = 49E2 variable light chain
QSALTQPASVSGSPGQSITISCTGTSSDVGIYNLVSWYQQHPGKAPKLMIHEVSKRPSGVSNRFSGSKSGNTASLTISGLQAEDEADYYCCSYAGISTWVFGGGTKLTVLG

SEQ ID NO: 394 - protein, 5 aa, synthetic construct
  REGION 1..5: note = 49E2 heavy chain CDR1
SYGMH

SEQ ID NO: 395 - protein, 17 aa, synthetic construct
  REGION 1..17: note = 49E2 heavy chain CDR2
VIWSDGNNKYYEDSVKG

SEQ ID NO: 396 - protein, 8 aa, synthetic construct
  REGION 1..8: note = 49E2 heavy chain CDR3
DTATPFDY

SEQ ID NO: 397 - protein, 14 aa, synthetic construct
  REGION 1..14: note = 49E2 light chain CDR1
TGTSSDVGIYNLVS

SEQ ID NO: 398 - protein, 7 aa, synthetic construct
  REGION 1..7: note = 49E2 light chain CDR2
EVSKRPS

SEQ ID NO: 399 - protein, 10 aa, synthetic construct
  REGION 1..10: note = 49E2 light chain CDR3
CSYAGISTWV

SEQ ID NO: 400 - protein, 452 aa, synthetic construct
  REGION 1..452: note = 48A9 heavy chain
QVQLVESGGGVVQPGRSLRLSCAASGFTFSSCGMHWVRQAPGKGLEWVAVISYDGSNKYYADSVKGRFTISRDNSKNTLYLQMNSLRAEDTAVYYCARDLRYNWNDGGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 401 - protein, 214 aa, synthetic construct
  REGION 1..214: note = 48A9 light chain
DIQMTQSPSSLSASVGDRVIITCRASQSISSYLHWYKQKPGKAPKLLIYGASRLQSGVPSRFSGSGSGTDFTLTISSLQPEDFATYYCQQSSSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 402 - protein, 122 aa, synthetic construct
  REGION 1..122: note = 48A9 variable heavy chain
QVQLVESGGGVVQPGRSLRLSCAASGFTFSSCGMHWVRQAPGKGLEWVAVISYDGSNKYYADSVKGRFTISRDNSKNTLYLQMNSLRAEDTAVYYCARDLRYNWNDGGVDYWGQGTLVTVSS

SEQ ID NO: 403 - protein, 108 aa, synthetic construct
  REGION 1..108: note = 48A9 variable light chain
DIQMTQSPSSLSASVGDRVIITCRASQSISSYLHWYKQKPGKAPKLLIYGASRLQSGVPSRFSGSGSGTDFTLTISSLQPEDFATYYCQQSSSTPLTFGGGTKVEIKR

SEQ ID NO: 404 - protein, 5 aa, synthetic construct
  REGION 1..5: note = 48A9 heavy chain CDR1
SCGMH

SEQ ID NO: 405 - protein, 17 aa, synthetic construct
  REGION 1..17: note = 48A9 heavy chain CDR2
VISYDGSNKYYADSVKG

SEQ ID NO: 406 - protein, 13 aa, synthetic construct
  REGION 1..13: note = 48A9 heavy chain CDR3
DLRYNWNDGGVDY

SEQ ID NO: 407 - protein, 11 aa, synthetic construct
  REGION 1..11: note = 48A9 light chain CDR1
RASQSISSYLH

SEQ ID NO: 408 - protein, 7 aa, synthetic construct
  REGION 1..7: note = 48A9 light chain CDR2
GASRLQS

SEQ ID NO: 409 - protein, 9 aa, synthetic construct
  REGION 1..9: note = 48A9 light chain CDR3
QQSSSTPLT

SEQ ID NO: 410 - protein, 455 aa, synthetic construct
  REGION 1..455: note = 5H7 heavy chain
QVQLQESGPGLVKPSQTLSLTCTVSGGSISSGGYFWSWIRQHPGKGLEWIGYIYYSGTTYYNPSLKSRVTISIDTSKNHFSLKLSSVTAADTAVYYCARDLFYYDSSGPRGFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 411 - protein, 215 aa, synthetic construct
  REGION 1..215: note = 5H7 light chain
EIVLTQSPGTLSLSPGERATLSCRASQTVSSNYLAWYQQKPGQAPRLLIYGSSTRATGIPDRFSGSGSGTDFTLTISRLEPEDFAVYYCQQYDSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 412 - protein, 125 aa, synthetic construct
  REGION 1..125: note = 5H7 variable heavy chain
QVQLQESGPGLVKPSQTLSLTCTVSGGSISSGGYFWSWIRQHPGKGLEWIGYIYYSGTTYYNPSLKSRVTISIDTSKNHFSLKLSSVTAADTAVYYCARDLFYYDSSGPRGFDPWGQGTLVTVSS

SEQ ID NO: 413 - protein, 109 aa, synthetic construct
  REGION 1..109: note = 5H7 variable light chain
EIVLTQSPGTLSLSPGERATLSCRASQTVSSNYLAWYQQKPGQAPRLLIYGSSTRATGIPDRFSGSGSGTDFTLTISRLEPEDFAVYYCQQYDSSPWTFGQGTKVEIKR

SEQ ID NO: 414 - protein, 7 aa, synthetic construct
  REGION 1..7: note = 5H7 heavy chain CDR1
SGGYFWS

SEQ ID NO: 415 - protein, 16 aa, synthetic construct
  REGION 1..16: note = 5H7 heavy chain CDR2
YIYYSGTTYYNPSLKS

SEQ ID NO: 416 - protein, 15 aa, synthetic construct
  REGION 1..15: note = 5H7 heavy chain CDR3
DLFYYDSSGPRGFDP

SEQ ID NO: 417 - protein, 12 aa, synthetic construct
  REGION 1..12: note = 5H7 light chain CDR1
RASQTVSSNYLA

SEQ ID NO: 418 - protein, 7 aa, synthetic construct
  REGION 1..7: note = 5H7 light chain CDR2
GSSTRAT

SEQ ID NO: 419 - protein, 9 aa, synthetic construct
  REGION 1..9: note = 5H7 light chain CDR3
QQYDSSPWT

SEQ ID NO: 420 - protein, 453 aa, synthetic construct
  REGION 1..453: note = 7A10 heavy chain
QVQLVESGGGVVQPGRSLRLSCAASGFTFSSYGMHWVRQAPGKGLEWMAVIWYVGSNKYYADSVKGRFTISRDNSKNTLYLQMNSLSAEDTAVYYCARGGELGRDYYSGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 421 - protein, 214 aa, synthetic construct
  REGION 1..214: note = 7A10 light chain
DIQMTQSPSSLSASVGDRVTITCRASQGIRNDLGWYQQKPGKAPKRLIYAASSLQSGVPSRFSGSGSGTEFTLTISSLQPEDFATYYCQQHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 422 - protein, 123 aa, synthetic construct
  REGION 1..123: note = 7A10 variable heavy chain
QVQLVESGGGVVQPGRSLRLSCAASGFTFSSYGMHWVRQAPGKGLEWMAVIWYVGSNKYYADSVKGRFTISRDNSKNTLYLQMNSLSAEDTAVYYCARGGELGRDYYSGMDVWGQGTTVTVSS

SEQ ID NO: 423 - protein, 108 aa, synthetic construct
  REGION 1..108: note = 7A10 variable light chain
DIQMTQSPSSLSASVGDRVTITCRASQGIRNDLGWYQQKPGKAPKRLIYAASSLQSGVPSRFSGSGSGTEFTLTISSLQPEDFATYYCQQHNSYPWTFGQGTKVEIKR

SEQ ID NO: 424 - protein, 5 aa, synthetic construct
  REGION 1..5: note = 7A10 heavy chain CDR1
SYGMH

SEQ ID NO: 425 - protein, 17 aa, synthetic construct
  REGION 1..17: note = 7A10 heavy chain CDR2
VIWYVGSNKYYADSVKG

SEQ ID NO: 426 - protein, 14 aa, synthetic construct
  REGION 1..14: note = 7A10 heavy chain CDR3
GGELGRDYYSGMDV

SEQ ID NO: 427 - protein, 11 aa, synthetic construct
  REGION 1..11: note = 7A10 light chain CDR1
RASQGIRNDLG

SEQ ID NO: 428 - protein, 7 aa, synthetic construct
  REGION 1..7: note = 7A10 light chain CDR2
AASSLQS

SEQ ID NO: 429 - protein, 9 aa, synthetic construct
  REGION 1..9: note = 7A10 light chain CDR3
QQHNSYPWT

SEQ ID NO: 430 - protein, 453 aa, synthetic construct
  REGION 1..453: note = 9H6 heavy chain
QVQLVESGGGVVQPGRSLRLSCVASGFTFSSYGMHWIRQAPGKGLEWVAVIWYEGSNKYYADSVKGRFTISRDNSKNTLYLQMNSLRAEDTAVYYCARGGRLGKDYYSGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 431 - protein, 214 aa, synthetic construct
  REGION 1..214: note = 9H6 light chain
DIQMTQSPSSLSASVGDRVTITCRASQGIRNDLGWYQQKPGKAPNRLIYATSSLQSGVPSRFSGSGSGTEFTLTISSLQPEDFATYYCLQHNTYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 432 - protein, 123 aa, synthetic construct
  REGION 1..123: note = 9H6 variable heavy chain
QVQLVESGGGVVQPGRSLRLSCVASGFTFSSYGMHWIRQAPGKGLEWVAVIWYEGSNKYYADSVKGRFTISRDNSKNTLYLQMNSLRAEDTAVYYCARGGRLGKDYYSGMDVWGQGTTVTVSS

SEQ ID NO: 433 - protein, 108 aa, synthetic construct
  REGION 1..108: note = 9H6 variable light chain
DIQMTQSPSSLSASVGDRVTITCRASQGIRNDLGWYQQKPGKAPNRLIYATSSLQSGVPSRFSGSGSGTEFTLTISSLQPEDFATYYCLQHNTYPWTFGQGTKVEIKR

SEQ ID NO: 434 - protein, 5 aa, synthetic construct
  REGION 1..5: note = 9H6 heavy chain CDR1
SYGMH

SEQ ID NO: 435 - protein, 17 aa, synthetic construct
  REGION 1..17: note = 9H6 heavy chain CDR2
VIWYEGSNKYYADSVKG

SEQ ID NO: 436 - protein, 14 aa, synthetic construct
  REGION 1..14: note = 9H6 heavy chain CDR3
GGRLGKDYYSGMDV

SEQ ID NO: 437 - protein, 11 aa, synthetic construct
  REGION 1..11: note = 9H6 light chain CDR1
RASQGIRNDLG

SEQ ID NO: 438 - protein, 7 aa, synthetic construct
  REGION 1..7: note = 9H6 light chain CDR2
ATSSLQS

SEQ ID NO: 439 - protein, 9 aa, synthetic construct
  REGION 1..9: note = 9H6 light chain CDR3
LQHNTYPWT

SEQ ID NO: 440 - protein, 177 aa, synthetic construct
  REGION 1..177: note = GITRL
MCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 441 - protein, 125 aa, synthetic construct
  REGION 1..125: note = GITRL soluble domain
TAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 442 - protein, 283 aa, Homo sapiens [sequence omitted]

SEQ ID NO: 443 - protein, 240 aa, synthetic construct
  REGION 1..240: note = LIGHT (HVEM ligand)
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 444 - protein, 147 aa, synthetic construct
  REGION 1..147: note = LIGHT soluble domain
PAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 445 - protein, 146 aa, synthetic construct
  REGION 1..146: note = LIGHT soluble domain (alternative)
AAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 446 - protein, 145 aa, synthetic construct
  REGION 1..145: note = LIGHT soluble domain (alternative)
AHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 447 - protein, 335 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 448 - protein, 314 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 449 - protein, 220 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPMLT

SEQ ID NO: 450 - protein, 197 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWGNSGNKFI

SEQ ID NO: 451 - protein, 132 aa, synthetic construct
  REGION 1..132: note = heavy chain variable region for E09
QLQLQESGPGLVKPSETLSLTCTVSGASISANSYYGVWVRQSPGKGLEWVGSIAYRGNSNSGSTYYNPSLKSRATVSVDTSKNQVSLRLTSVTAADTALYYCARRQLLDDGTGYQWAAFDVWGQGTMVTVSS

SEQ ID NO: 452 - protein, 110 aa, synthetic construct
  REGION 1..110: note = light chain variable region for E09
QSVLTQPPSVSEAPRQTVTISCSGNSFNIGRYPVNWYQQLPGKAPKLLIYYNNLRFSGVSDRFSGSKSGTSASLAIRDLLSEDEADYYCSTWDDTLKGWVFGGGTKVTVL

SEQ ID NO: 453 - protein, 7 aa, synthetic construct
  REGION 1..7: note = heavy chain CDRl for E09
ANSYYGV

SEQ ID NO: 454 - protein, 22 aa, synthetic construct
  REGION 1..22: note = heavy chain CDR2 for E09
GSIAYRGNSNSGSTYYNPSLKS

SEQ ID NO: 455 - protein, 17 aa, synthetic construct
  REGION 1..17: note = heavy chain CDR3 for E09
RQLLDDGTGYQWAAFDV

SEQ ID NO: 456 - protein, 13 aa, synthetic construct
  REGION 1..13: note = light chain CDR1 for E09
SGNSFNIGRYPVN

SEQ ID NO: 457 - protein, 7 aa, synthetic construct
  REGION 1..7: note = light chain CDR2 for E09
YNNLRFS

SEQ ID NO: 458 - protein, 11 aa, synthetic construct
  REGION 1..11: note = light chain CDR3 for E09
STWDDTLKGWV

SEQ ID NO: 459 - protein, 281 aa, synthetic construct
  REGION 1..281: note = CD95L (CD95 ligand)
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 460 - protein, 136 aa, synthetic construct
  REGION 1..136: note = CD95L soluble domain
VAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 461 - protein, 135 aa, synthetic construct
  REGION 1..135: note = CD95L soluble domain (alternative)
AHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 462 - protein, 134 aa, synthetic construct
  REGION 1..134: note = CD95L soluble domain (alternative)
HLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 463 - protein, 440 aa, synthetic construct
  REGION 1..440: note = nivolumab heavy chain
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 464 - protein, 214 aa, synthetic construct
  REGION 1..214: note = nivolumab light chain
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 465 - protein, 113 aa, synthetic construct [sequence omitted]

SEQ ID NO: 466 - protein, 107 aa, synthetic construct [sequence omitted]

SEQ ID NO: 467 - protein, 5 aa, synthetic construct
  REGION 1..5: note = nivolumab heavy chain CDR1
NSGMH

SEQ ID NO: 468 - protein, 17 aa, synthetic construct
  REGION 1..17: note = nivolumab heavy chain CDR2
VIWYDGSKRYYADSVKG

SEQ ID NO: 469 - protein, 4 aa, synthetic construct
  REGION 1..4: note = nivolumab heavy chain CDR3
NDDY

SEQ ID NO: 470 - protein, 11 aa, synthetic construct
  REGION 1..11: note = nivolumab light chain CDR1
RASQSVSSYLA

SEQ ID NO: 471 - protein, 7 aa, synthetic construct
  REGION 1..7: note = nivolumab light chain CDR2
DASNRAT

SEQ ID NO: 472 - protein, 9 aa, synthetic construct
  REGION 1..9: note = nivolumab light chain CDR3
QQSSNWPRT

SEQ ID NO: 473 - protein, 447 aa, synthetic construct
  REGION 1..447: note = pembrolizumab heavy chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 474 - protein, 218 aa, synthetic construct
  REGION 1..218: note = pembrolizumab light chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 475 - protein, 120 aa, synthetic construct [sequence omitted]

SEQ ID NO: 476 - protein, 111 aa, synthetic construct [sequence omitted]

SEQ ID NO: 477 - protein, 5 aa, synthetic construct
  REGION 1..5: note = pembrolizumab heavy chain CDR1
NYYMY

SEQ ID NO: 478 - protein, 16 aa, synthetic construct
  REGION 1..16: note = pembrolizumab heavy chain CDR2
GINPSNGGTNFNEKFK

SEQ ID NO: 479 - protein, 11 aa, synthetic construct
  REGION 1..11: note = pembrolizumab heavy chain CDR3
RDYRFDMGFDY

SEQ ID NO: 480 - protein, 15 aa, synthetic construct
  REGION 1..15: note = pembrolizumab light chain CDR1
RASKGVSTSGYSYLH

SEQ ID NO: 481 - protein, 7 aa, synthetic construct
  REGION 1..7: note = pembrolizumab light chain CDR2
LASYLES

SEQ ID NO: 482 - protein, 9 aa, synthetic construct
  REGION 1..9: note = pembrolizumab light chain CDR3
QHSRDLPLT

SEQ ID NO: 483 - protein, 451 aa, synthetic construct
  REGION 1..451: note = durvalumab heavy chain
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 484 - protein, 265 aa, synthetic construct
  REGION 1..265: note = durvalumab light chain
EVQLVESGGGLVQPGGSLRLSCAASGFTFSRYWMSWVRQAPGKGLEWVANEIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 485 - protein, 121 aa, synthetic construct
  REGION 1..121: note = durvalumab variable heavy chain
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 486 - protein, 108 aa, synthetic construct
  REGION 1..108: note = durvalumab variable light chain
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 487 - protein, 5 aa, synthetic construct
  REGION 1..5: note = durvalumab heavy chain CDR1
RYWMS

SEQ ID NO: 488 - protein, 17 aa, synthetic construct
  REGION 1..17: note = durvalumab heavy chain CDR2
NIKQDGSEKYYVDSVKG

SEQ ID NO: 489 - protein, 12 aa, synthetic construct
  REGION 1..12: note = durvalumab heavy chain CDR3
EGGWFGELAFDY

SEQ ID NO: 490 - protein, 12 aa, synthetic construct
  REGION 1..12: note = durvalumab light chain CDR1
RASQRVSSSYLA

SEQ ID NO: 491 - protein, 7 aa, synthetic construct
  REGION 1..7: note = durvalumab light chain CDR2
DASSRAT

SEQ ID NO: 492 - protein, 9 aa, synthetic construct
  REGION 1..9: note = durvalumab light chain CDR3
QQYGSLPWT

SEQ ID NO: 493 - protein, 450 aa, synthetic construct
  REGION 1..450: note = avelumabheavy chain
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 494 - protein, 216 aa, synthetic construct
  REGION 1..216: note = avelumablight chain
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 495 - protein, 120 aa, synthetic construct
  REGION 1..120: note = avelumabvariable heavy chain
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 496 - protein, 110 aa, synthetic construct
  REGION 1..110: note = avelumabvariable light chain
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 497 - protein, 5 aa, synthetic construct
  REGION 1..5: note = avelumabheavy chain CDR1
SYIMM

SEQ ID NO: 498 - protein, 17 aa, synthetic construct
  REGION 1..17: note = avelumabheavy chain CDR2
SIYPSGGITFYADTVKG

SEQ ID NO: 499 - protein, 11 aa, synthetic construct
  REGION 1..11: note = avelumabheavy chain CDR3
IKLGTVTTVDY

SEQ ID NO: 500 - protein, 14 aa, synthetic construct
  REGION 1..14: note = avelumablight chain CDR1
TGTSSDVGGYNYVS

SEQ ID NO: 501 - protein, 7 aa, synthetic construct
  REGION 1..7: note = avelumablight chain CDR2
DVSNRPS

SEQ ID NO: 502 - protein, 10 aa, synthetic construct
  REGION 1..10: note = avelumablight chain CDR3
SSYTSSSTRV

SEQ ID NO: 503 - protein, 448 aa, synthetic construct
  REGION 1..448: note = atezolizumabheavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 504 - protein, 214 aa, synthetic construct
  REGION 1..214: note = atezolizumablight chain
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 505 - protein, 118 aa, synthetic construct
  REGION 1..118: note = atezolizumabvariable heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 506 - protein, 108 aa, synthetic construct
  REGION 1..108: note = atezolizumabvariable light chain
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 507 - protein, 10 aa, synthetic construct
  REGION 1..10: note = atezolizumabheavy chain CDR1
GFTFSDSWIH

SEQ ID NO: 508 - protein, 18 aa, synthetic construct
  REGION 1..18: note = atezolizumabheavy chain CDR2
AWISPYGGSTYYADSVKG

SEQ ID NO: 509 - protein, 9 aa, synthetic construct
  REGION 1..9: note = atezolizumabheavy chain CDR3
RHWPGGFDY

SEQ ID NO: 510 - protein, 11 aa, synthetic construct
  REGION 1..11: note = atezolizumablight chain CDR1
RASQDVSTAVA

SEQ ID NO: 511 - protein, 7 aa, synthetic construct
  REGION 1..7: note = atezolizumablight chain CDR2
SASFLYS

SEQ ID NO: 512 - protein, 9 aa, synthetic construct
  REGION 1..9: note = atezolizumablight chain CDR3
QQYLYHPAT